(19) United States
(12) Patent Application Publication
(10) Pub. No.: US 2026/0034112 A1
(43) Pub. Date: Feb. 5, 2026

(54) MULTI-CYCLIC IRAK1 AND IRAK4 INHIBITING COMPOUNDS AND USES THEREOF

(71) Applicants: CHILDREN'S HOSPITAL MEDICAL CENTER, Cincinnati, OH (US); THE UNITED STATES OF AMERICA, AS REPRESENTED BY THE SECRETARY, DEPT. OF HEALTH AND HUMAN SERVICES, Bethesda, MD (US); KUROME THERAPEUTICS, INC., Cincinnati, OH (US)

(72) Inventors: Craig Joseph THOMAS, Gaithersburg, MD (US); Scott Bryan HOYT, Arlington, VA (US); Daniel T. STARCZYNOWSKI, Cincinnati, OH (US); Jan Susan ROSENBAUM, Cincinnati, OH (US); Joshua BENNETT, Cincinnati, OH (US)

(73) Assignees: CHILDREN'S HOSPITAL MEDICAL CENTER, Cincinnati, OH (US); THE UNITED STATES OF AMERICA, AS REPRESENTED BY THE SECRETARY, DEPT. OF HEALTH AND HUMAN SERVICES, Bethesda, MD (US); KUROME THERAPEUTICS, INC., Cincinnati, OH (US)

(21) Appl. No.: 19/099,849

(22) PCT Filed: Aug. 1, 2023
(86) PCT No.: PCT/US2023/071435

§ 371 Date: Jan. 30, 2025

Related U.S. Application Data
(60) Provisional application No. 63/394,118, filed on Aug. 1, 2022.
(60) Provisional application No. 63/377,812, filed on Sep. 30, 2022.
(60) Provisional application No. 63/487,079, filed on Feb. 27, 2023.

(51) Int. Cl. A61K 31/444; A61P 35/02

(52) U.S. Cl. CPC A61K 31/444; A61P 35/02

(57) ABSTRACT

The present disclosure provides a method of treating an inflammatory disease/disorder, acute myeloid leukemia (AML), or myelodysplastic syndrome (MDS) in a subject comprising administering to the subject a compound that inhibits IRAK1 and IRAK4. The present disclosure further provides a method of determining a compound that is effective at treating an inflammatory disease/disorder, AML, or MDS.

### CROSS-REFERENCE TO RELATED APPLICATIONS

The present application claims priority to U.S. Provisional Application No. 63/394,118, filed Aug. 1, 2022, U.S. Provisional Application No. 63/377,812, filed Sep. 30, 2022, and U.S. Provisional Application No. 63/487,079, filed Feb. 27, 2023, each of which is incorporated by reference herein in its entirety.

### GOVERNMENT RIGHTS

This invention was made in the performance of a Cooperative Research and Development Agreement with the National Institutes of Health, an Agency of the Department of Health and Human Services. The Government of the United States has certain rights in this invention.

### BACKGROUND

IRAKs are a family of related kinases that operate at the nexus of multiple innate immune and inflammatory pathways implicated in myeloid malignancies. There is increasing interest in the role of the IRAK kinases as targets in the treatment of myelodysplastic syndromes (MDS) and acute myeloid leukemia (AML) (reviewed in J Bennett and DT Starczynowski, Curr Opin Hematol 2022). IRAK1 and IRAK4 lie downstream of multiple receptors that stimulate the canonical NF-kB signaling pathway upstream of TRAF6 via the myddosome complex. While TRAF6 is necessary to preserve the tonic NF-kB signaling that is required for hematopoietic stem cell (HSC) homeostasis (J Fang et. al. Cell Reports 2018), overactivity of the NF-kB signaling pathway has been implicated in both MDS and AML (reviewed in M C J Bosman et. al., Crit Rev Oncol/Hematol 2016; JJ Trowbridge and DT Starczynowski, J Exp Med 2021). The requirement for both IRAK1 and IRAK4 in this pathway has been explored using both genetic and pharmacologic technologies. The use of genetic loss-of-function approaches revealed that deletion and/or inhibition of IRAK4 results in a compensatory increase in IRAK1 protein and activation (data unpublished), suggesting that high potency antagonism of both kinases will be required for optimal inhibition of NF-kB-mediated transcriptional responses in disease-propagating MDS and AML cells. IRAK4 inhibitors have advanced into clinical trials for MDS and AML validating IRAK4 as a therapeutic target. Early data from these trials has been encouraging; however, the overall responses remain modest.

MDS and AML exist along a continuous disease spectrum starting with early-stage MDS, which may progress to high-risk (HR) MDS and/or AML. AML is a heterogeneous malignancy characterized by suppression of normal hematopoiesis and overproduction of immature myeloid blast cells associated with a differentiation block. Most patients with HR-MDS and AML are not cured with available therapies, underscoring the urgent need for new therapeutic alternatives that will improve the clinical outcomes of these patients. Despite significant effort, the five-year relative survival of AML patients remains at only 25%. MDS and AML originate in hematopoietic stem and progenitor cells (HSPC, referred to as leukemic stem/progenitor cells [LSPC]) that acquire genetic and/or epigenetic abnormalities. A pool of LSPCs persist throughout the course of disease to replenish the bulk leukemic blast cells and contribute to treatment-related relapse when not eradicated. Recent therapies, such as Venetoclax with Azacytidine and Menin inhibitors, that target the LSPCs have shown improved clinical outcomes for patients with HR-MDS and AML. LSPCs also share cellular states and transcriptional programs with normal HSPCs, such as ones that maintain multi-potent self-renewal properties and prevent untimely differentiation. Like normal HSPCs, LSPCs have acquired an undifferentiated state that permits long-term expansion of progeny cells, properties which have been evaluated using surrogate in vitro progenitor assays (i.e., colony assays in methylcellulose) and in vivo hematopoietic transplantation models.

Uncovering targetable signaling dependencies unique to LSPCs is critical to improve therapeutic responses in patients. It was recently reported that patient-derived LSPCs from HR-MDS and across various AML subtypes exhibit a high frequency of dysregulated immune and inflammatory pathways. Dysregulation of immune-related genes is observed in >50% of MDS and AML, and chronic innate immune pathway activation increases the risk of developing myeloid malignancies. Moreover, a significant number of genetic and molecular alterations in MDS and AML directly impinge on effectors of the Toll-like receptor (TLR) and Interleukin 1 receptor (IL-1R) pathways, both of which converge on a signaling complex operated by the interleukin-1 receptor associated kinases (IRAK): IRAK1, IRAK2, and IRAK4. In the context of normal immune cell biology, activation of TLRs or IL-1R leads to recruitment of the adaptor protein MyD88, which then nucleates an oligomeric signaling complex (Myddosome) that includes MyD88 and IRAK4. IRAK4 subsequently recruits and phosphorylates IRAK1 or IRAK2, which then induces multiple downstream effectors, including NF-kB and MAPKs.

Chronic activation of this MyD88-IRAK canonical signaling axis is presumed to underly malignant hematopoiesis in MDS/AML, a theory that is partially supported by the discovery of activating mutations in MyD88 that cause spontaneous assembly of the Myddosome complex in lymphomas. Consequently, IRAK4 inhibitors and proteolysis targeting chimeric (PROTAC) small molecule degraders are undergoing evaluation in pre-clinical studies and early phase clinical trials for hematologic malignancies and inflammatory conditions. The IRAK4 kinase inhibitor PF-06650833 (Zimlovisertib) is being evaluated for chronic inflammatory disorders, while CA-4948 (Emavusertib) is being evaluated in hematologic malignancies, including lymphoma, HR- and low-risk (LR)-MDS, and refractory/relapsed AML. The IRAK4 PROTACs, KT-474 and KT-413, are being evaluated in immuno-inflammatory disease and MyD88-mutant lymphomas, respectively. Safety data from these trials have revealed minimal on-target toxicity nor adverse effects, suggesting that targeting IRAK4 will have an acceptable safety profile and tolerability. Initial results from the early phase trials in HR-MDS and AML with CA-4948 showed complete or partial response rates of ˜40% and additional patients having reductions in leukemic blast counts. Interestingly, 40% of AML patients with spliceosome mutations reached a complete response (CR) or CR with partial hematologic recovery (CRh), while 57% of HR-MDS patients achieved a CR. These clinical observations support the recent findings that mutations in the splicing factors U2AF1 and SF3B1 directly induce hypermorphic IRAK4 isoforms and active innate immune signaling in MDS and AML. The data emerging from the ongoing clinical studies suggest that IRAK4 is a relevant target in hematologic malignancies. However, the magnitude of clinical responses appears to depend on genetic background and suggests inadequacy of IRAK4 inhibitors as monotherapy. Thus, a better understanding of IRAK4 signaling is necessary to refine therapeutic strategies targeting dysregulated innate immune and inflammatory signaling in hematologic malignancies.

The present disclosure addresses this unmet need.

### SUMMARY OF THE DISCLOSURE

In one aspect, the present disclosure provides a method of treating an inflammatory disease/disorder, acute myeloid leukemia (AML), or myelodysplastic syndrome (MDS) in a subject in need thereof, the method comprising administering to the subject a compound that inhibits IRAK1 and IRAK4. In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the AML is relapsed AIL, refractory AML, relapsed/refractory AML, AML with resistance to hypomethylating agents, AML with resistance to venetoclax, AML with resistance to hypomethylating agents and venetoclax, monocytic AML, or monocytic-like AML. In one embodiment, the MDS is MDS with a splicing factor mutation, MDS with a mutation in isocitrate dehydrogenase 1, or MDS with a mutation in isocitrate dehydrogenase 2. In one embodiment, the MDS with a splicing factor mutation comprises MDS with a splicing factor mutation in U2AF1, SRSF2, SF3B1, or ZRSR2. In one embodiment, the subject in need thereof has elevated IRAK1 expression, IRAK4 expression, FLT3 expression, NF-kB signaling, TLR signaling, IL-1 signaling, or a combination thereof. In one embodiment, the subject in need thereof has elevated IRAK1 expression, IRAK4 expression, or a combination thereof. In one embodiment, the subject in need thereof has elevated FLT3 expression. In one embodiment, the compound has an IRAK1 IC₅₀ of less than about 75 nM. In one embodiment, the compound has an IRAK4 IC₅₀ of less than about 10 nM. In one embodiment, the compound has IRAK4:IRAK1 potency ratio of less than about 40. In one embodiment, the subject in need thereof has elevated expression of one or more IRAK1/4-associated genes compared to a healthy control subject and/or decreased expression of one or more IRAK1/4-associated genes compared to a healthy control subject. In one embodiment, the subject in need thereof has elevated expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIMI1K, COMTD, ECHDC3, PAOX, LPCAT1, ARSG, PSTPIP2, VENTX, AMIGO2, PCED1B, PCED1B-AS1, and BAG3 compared to the healthy control subject. In one embodiment, the subject in need thereof has decreased expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDL1M1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject. In one embodiment, the compound decreases the expression of one of more IRAK1/4-associated genes found to be elevated in the subject compared to the healthy control subject and/or increases the expression of one of more IRAK1/4-associated genes found to be decreased in the subject compared to the healthy control subject. In one embodiment, the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF1, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one or more IRAK1/4-associated genes selected from: KRAS, SOX5, EMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound is more effective at inhibiting cancer cell colony formation and/or cancer progenitor cell function when compared to a compound which inhibits IRAK1 without inhibiting IRAK4 or inhibits IRAK4 without inhibiting IRAK1. In one embodiment, the compound provides sustained treatment of the inflammatory disease/disorder, AML, or MDS in the subject in need thereof. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in the subject in need thereof after the administration is stopped. In one embodiment, the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (II-5010), (IIa-5010), (JIb-5010), (III-5010), (IIIa-5010), (IIIb-5010), or a salt, ester, solvate, optical isomer, geometric isomer, or salt of an isomer of any one thereof.

In another aspect, the present disclosure provides a method of determining effectiveness of a compound in treating an inflammatory disease/disorder, acute myeloid leukemia (AML), or myelodysplastic syndrome (MDS), comprising:
determining the compound's IRAK1 IC₅₀ and IRAK4 IC₅₀;
calculating an IRAK4:RAK1 potency ratio from the IRAK1 IC₅₀ and the IRAK4 IC₅₀ of the compound;
wherein a potency ratio of less than about 40 is indicative of effectiveness of the compound in treating the inflammatory disease/disorder, AML, or MDS. In one embodiment, the method further administering the compound to a subject with an inflammatory disease/disorder, AML, or MDS, treating the inflammatory disease/disorder, AML, or MDS. In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the AML is relapsed AML, refractory AML, relapsed/refractory AML, AML with resistance to hypomethylating agents, AML with resistance to venetoclax, AML with resistance to hypomethylating agents and venetoclax, monocytic AIL, or monocytic-like AML. In one embodiment, the MDS is MDS with a splicing factor mutation, MDS with a mutation in isocitrate dehydrogenase 1, or MDS with a mutation in isocitrate dehydrogenase 2. In one embodiment, the MDS with a splicing factor mutation comprises MDS with a splicing factor mutation in U2AF1, SRSF2, SF3B1, or ZRSR2. In one embodiment, the compound has an IRAK1 IC₅₀ of less than about 75 nM. In one embodiment, the compound has an IRAK4 IC₅₀ of less than about 10 nM. In one embodiment, the compound is more effective at inhibiting cancer cell colony formation and/or cancer progenitor cell function when compared to a compound which inhibits IRAK1 without inhibiting IRAK4 or inhibits IRAK4 without inhibiting IRAK1. In one embodiment, the compound provides sustained treatment of the inflammatory disease/disorder, AML, or MDS when administered to a subject in need thereof. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in a subject in need thereof after administration of the compound to the subject is stopped. In one embodiment, the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

In yet another aspect, the present disclosure provides a method of determining effectiveness of a compound in treating an inflammatory disease/disorder, acute myeloid leukemia (AML), or myelodysplastic syndrome (MDS), comprising:
determining that the compound is an IRAK1/4 inhibitor which modifies the expression of one of more IRAK1/4-associated genes found to be differentially expressed in a subject with an inflammatory disease/disorder, AML, or MDS compared to a healthy control subject. In one embodiment, the method further comprises administering the compound to the subject, treating the inflammatory disease/disorder, AML, or MDS. In one embodiment, the compound decreases the expression of one of more IRAK1/4-associated genes found to be elevated in the subject compared to the healthy control subject and/or increases the expression of one of more IRAK1/4-associated genes found to be decreased in the subject compared to the healthy control subject. In one embodiment, the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the AML is relapsed AML, refractory AIL, relapsed/refractory AML, AML with resistance to hypomethylating agents, AML with resistance to venetoclax, AIL with resistance to hypomethylating agents and venetoclax, monocytic AML, or monocytic-like AML. In one embodiment, the MDS is MDS with a splicing factor mutation, MDS with a mutation in isocitrate dehydrogenase 1, or MDS with a mutation in isocitrate dehydrogenase 2. In one embodiment, the MDS with a splicing factor mutation comprises MDS with a splicing factor mutation in U2AF1, SRSF2, SF3B1, or ZRSR2. In one embodiment, the compound has an IRAK1 IC₅₀ of less than about 75 nM. In one embodiment, the compound has an IRAK4 IC₅₀ of less than about 10 nM. In one embodiment, the compound is more effective at inhibiting cancer cell colony formation and/or cancer progenitor cell function when compared to a compound which inhibits IRAK1 without inhibiting IRAK4 or inhibits IRAK4 without inhibiting IRAK1. In one embodiment, the compound provides sustained treatment of the inflammatory disease/disorder, AML, or MDS when administered to a subject in need thereof. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in a subject in need thereof after administration of the compound to the subject is stopped. In one embodiment, the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (IIf-5009)-(IIj-5009), (1-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

### BRIEF DESCRIPTION OF THE DRAWINGS

FIG. 1 depicts that the NFkB potency of Compounds 1-13, Compound 15, and comparative compounds correlates with their IRAK1 potency.

FIG. 2 depicts that the NFkB potency of Compounds 1-13, Compound 15, and comparative compounds correlates with their IRAK4 potency.

FIG. 3 depicts colony forming assays demonstrating that the NFkB potency of Compounds 1-13 and Compound 15 correlates with their MDSL CFC potency.

FIG. 4 depicts colony forming assays demonstrating that the NFkB potency of Compounds 1-13 and Compound 15 correlates with their THP-1 CFC potency.

FIG. 5 depicts the kinase profile of comparative compounds with their PAM and IL1β IC₅₀.

FIG. 6A depicts Compound 14 and Compound 15. FIG. 6B depicts the kinase profile of Compound 14 and Compound 15 with their PAM and IL1β IC₅₀.

FIG. 7 depicts the kinase profile of Compound 2, Compound 7, and Compound 8 with their PAM and IL1β IC₅₀.

FIGS. 8A-8C depict an evaluation of IRAK4 inhibitors on AML cells. FIG. 8A: IC₅₀ curves for CA-4948 and PF-06650833 in an assay measuring NF-kB activity upon TLR2 stimulation with PAM3CSK4 in THP1-Blue NF-kB SEAP reporter cells. FIG. 8B: Colony formation counts of THP-1 cells treated with 1 or 10 μM PF-06650833 or vehicle control. FIG. 8C: Growth curves of THP-1 and MDS-L cells treated with 5 or 10 μM CA-4948 or DMSO.

FIGS. 9A-9N depict that IRAK4 inhibition causes activation of IRAK1. FIG. 9A: Colony formation in a panel of MDS/AML cell lines and patient-derived samples treated with the indicated concentrations of CA-4948 (two independent experiments). FIG. 9B: Immunoblots for IRAK4 in WT and IRAK4^{KO} AML cell lines and patient-derived samples. FIG. 9C: Colony formation of WT and IRAK4^{KO} AML cell lines and patient-derived samples. FIG. 9D: Experimental overview. RNA sequencing was performed on WT and IRAK4^{KO} THP1 cells, and THP1 cells treated for 24 hours with the indicated inhibitors. Genes upregulated upon IRAK4 deficiency or chemical inhibition were used to annotate compensatory pathways. FIG. 9E: Venn diagrams of overlapping upregulated genes upon IRAK4 deficiency or IRAK4 chemical inhibition. FIG. 9F: Heatmap of differentially expressed genes upon treatment with IRAK4 inhibitors (fold-change >1.5; P<0.05). FIG. 9G: Heatmap of differentially expressed genes upon treatment with IRAK4-degrader 1 (fold-change >1.5; P<0.05) or upon IRAK4 deletion (fold-change >2.0; P<0.05). FIG. 9H: Pathway enrichment of KEGG datasets using overlapping genes increased upon treatment with IRAK4 inhibitors. FIG. 9I: Pathway enrichment of KEGG datasets using overlapping genes increased upon treatment with IRAK4-degrader 1 or following deletion of IRAK4. FIG. 9J: Overview of canonical Myd88-dependent signaling: Upon TLR ligation, MyD88 nucleates a complex with IRAK4, which signals through IRAK1 and/or IRAK2 and then TRAF6 to activate the NF-kB and MAPK pathways. FIG. 9K: Immunoblots for IRAK1, IRAK2, TRAF6, and MyD88 in WT and IRAK4KO AML cell lines and patient-derived samples. FIG. 9L: Immunoblots for phoshpo-IRAK1, total IRAK1, and IRAK4 in WT and IRAK4KO cell lines. FIG. 9M: Immunoblots for phoshpo-IRAK1, total IRAK1, and IRAK4 in MDSL and THP1 cells treated for 24 hours with IRAK4-degrader 1. FIG. 9N: Immunoblots for phospho-IRAK1, total IRAK1, IRAK2, and IRAK4 in MDSL and AML(1714) treated for 24 hours with CA-4948 (10 μM). Significance was determined with a Student's t test (*, P<0.05; **, P<0.01). Error bars represent the standard deviation.

FIGS. 10A-10F depict an evaluation of IRAK1/4-deficient AML cells. FIG. 10A: Immunoblots for IRAK1 and IRAK4 in WT and IRAK1^{KO} THP1 and MDSL clones. FIG. 10B: Growth curves of WT and IRAK1^{KO} MDSL (10 μM) and THP1 (20 μM) treated with PF-06650833 or DMSO (two independent experiments). FIG. 10C: Growth curve of WT, IRAK4^{KO}, IRAK1^{KO} and IRAK1/4^{dKO} THP1 cells. FIG. 10D: Immunoblots for phospho-p38 MAPK, phospho-ERK1/2, phospho-IKKa/b, and phospho-JNK in WT, IRAK4^{KO}, RAK1^{KO}, and IRAK1/4^{dKO} THP1 cells treated with IL-1b (10 ng/ml) as compared to DMSO. FIG. 10E: Immunoblots for phospho-p38, phospho-ERK1/2, phospho-IKKa/b, and phospho-JNK in WT, IRAK4^{KO} IRAK1^{KO} and IRAK1/4^{dKO} THP1 cells treated with PAM3CSK4 (1 ug/ml) as compared to DMSO. FIG. 10F: Immunoblots for phospho-p38 MAPK, phospho-ERK1/2, phospho-IKKa/b, and phospho-JNK in WT, IRAK4^{KO}, IRAK1^{KO} and IRAK1/4^{dKO} MDSL cells treated with IL-1b (10 ng/ml) as compared to DMSO.

FIGS. 11A-11K depict that the inhibition of IRAK1 confers an exaggerated leukemic defect to IRAK4-deficient AML. FIG. 11A: Growth curves of WT and IRAK1^{KO} MDSL and THP1 cells treated with CA-4948 (10 μM) or vehicle (two independent experiments). FIG. 11B: Colony formation of WT and IRAK1^{KO}MDSL and THP1 cells treated with CA-4948 (30 μM) or vehicle (three independent experiments). FIG. 11C: Colony formation of WT and IRAK1^{KO} MDSL and THP1 cells treated with IRAK4-degrader 1 (MDSL, 5 μM; THP1, 10 μM) or vehicle. FIG. 11D: Immunoblots for IRAK1 and IRAK4 in WT and IRAK4^{KO} cell lines transduced with non-targeting control shRNA (shControl) or shIRAK1. FIG. 11E: Colony formation of WT and IRAK4^{KO} AML cell lines transduced with non-targeting control shRNA (shControl) or shIRAK1. FIG. 11F: Representative colony images of WT and IRAK4^{KO} AML(1294) cells transduced with non-target control shRNA (shControl) or shIRAK1. FIG. 11G: Immunoblots for IRAK1 and IRAK4 in WT, IRAK4^{KO}, IRAK1^{KO}, and IRAK1/4^{dKO} THP1 cells. FIG. 11H: Kaplan Meier survival analysis of NSGS mice (n=7 mice/group) engrafted with WT, IRAK4^{KO}, IRAK1^{KO}, and IRAK1/4^{dKO} THP1 cells (Data represent one of two independent experiments with identical trends). FIG. 11I: Immunoblots for IRAK1 and IRAK4 in WT, IRAK4^{KO}, IRAK1^{KO}, and IRAK1/4^{dKO} THP1 cells. FIG. 11J: Bone marrow engraftment of WT (n=4), IRAK4^{KO} (n=5), IRAK1^{KO} (n=5), and IRAK1/4^{dKO} (n=5) THP1 cells in xenografted NSGS mice at time of death. Leukemic engraftment was determined as percentage of huCD45⁺huCD33⁺ cells. FIG. 11K: Representative images of livers collected from NSGS mice xenografted with WT, IRAK4^{KO}, IRMA^{KO} and IRAK1/4^{dkO} THP1 cells. Arrows indicate examples of THP1 cells infiltration. Significance was determined with a Student's t test (*, P<0.05; **, P<0.01). Error bars represent the standard deviation.

FIGS. 12A-12C depict that MyD88 is dispensable for LSPCs. FIG. 12A: Immunoblots for MyD88 and activation of downstream pathways (phospho-p38, phospho-JNK, phospho-IKK, phospho-ERK) in WT and MYD88^{KO} THP1 cells upon 30 minutes treatment with IL-1b (10 ng/ul) or the TLR1/2 ligand PAM3CSK4 (1 ug/ml) as compared to DMSO. FIG. 12B: Immunoblots for IRAK4 and MyD88 in WT and MYD88^{KO} THP1 and MDSL cells transduced with non-targeting shRNA (shControl) or shIRAK4. FIG. 12C: Colony formation of WT and MYD88^{KO} THP1 and MDSL cells transduced with non-targeting shRNA (shControl) or shIRAK4. Significance was determined with a Student's t test (*, P<0.05). Error bars represent the standard deviation.

FIGS. 13A-13C depict an evaluation of TRAF6-deficient AML cells. FIG. 13A: Immunoblots for TRAF6 in WT and TRAF6^{KO} THP1 cells. FIG. 13B: Colony formation of WT and TRAF6^{KO} THP1 cells. FIG. 13C: Representative colony images of WT and TRAF6^{KO} THP1 cells.

FIGS. 14A-14G depict that non-canonical IRAK1/4 signaling is essential for maintaining LSPCs in MDS/AML. FIG. 14A: Principal component analysis of gene expression profiles of WT, IRAK4^{KO}, RAK1^{KO} and IRAK1/4^{dKO} THP1 cells. FIG. 14B: Volcano plots of differentially expressed genes in IRAK4^{KO} IRAK1^{KO} and IRAK1/4^{dKO} THP1 cells relative to WT THP1 cells (>2-fold change; P<0.05). FIG. 14C: Heatmap of differentially expressed genes in IRAK4^{KO}, IRAK4^{KO} and IRAK1/4^{dKO} THP1 relative to WT. Bars on the right denote differentially expressed genes attributed to deficiency of IRAK4 (red), IRAK1 (orange), or are unique to IRAK1/4^{dKO} (blue). FIG. 14D: Venn diagrams of overlapping upregulated or downregulated genes upon in IRAK4^{KO}, IRAK1^{KO} and IRAK1/4^{dKO} relative to WT THP1. FIG. 14E: Pathway enrichment of KEGG datasets of upregulated and downregulated genes in IRAK4^{K}, IRAK1^{KO} and IRAK1/4^{dKO} THP1 cells. FIG. 14F: Gene set enrichment analysis (GSEA) of genes dysregulated in IRAK1/4^{dKO} versus WT THP1 cells. Absolute normalized enrichment score (NES) and corresponding P value is shown for each pathway. FIG. 14G: Representative Wright-Giemsa stains of WT and IRAK4^{KO} THP1, MDSL, TF1, and AML(1294) expressing non-targeting shRNA (shControl) and shIRAK1, respectively.

FIGS. 15A-15D depict an evaluation of MyD88-deficient AML cells. FIG. 15A: Volcano plots based on bulk RNA-sequencing showing differentially expressed genes in MYD88^{KO} relative to WT THP1 cells (>2-fold, P<0.05). FIG. 15B: Heatmap of differentially expressed genes in MYD88^{KO} relative to WT THP1 cells. FIG. 15C: Pathway enrichment using KEGG of upregulated and downregulated genes in MYD88^{KO} relative to WT THP1 cells. Bar plots depict pathways ranked by −Log(P value) of enrichment score. FIG. 15D: Representative Wright-Giemsa stains of WT and MYD88^{KO} THP1 and MDSL cells.

FIGS. 16A-16H depict that the IRAK1 and IRAK4 interactomes reveal non-canonical signaling in AML. FIG. 16A: Experimental overview of RAK1 and IRAK4 proximity labeling in THP1 cells: Doxycycline-inducible IRAK4- and IRAK1-APEX2 fusion constructs were transduced into IRAK4^{KO} and IRAK1^{KO} THP1 cells, respectively. Functional rescue of canonical signaling was confirmed in NF-kB assays. Biotin phenol was added to induce proximity labeling with biotin. Biotinylated proteins were isolated and identified by mass-spectrometry. FIG. 16B: Venn diagram of unique and overlapping proteins in the IRAK4 and IRAK1 proximal proteins. FIG. 16C: Pathway enrichment using IRAK4-specific proximal proteins. Bars represent the number of IRAK4 interacting proteins that appear in the designated pathway. Dots represent −Log(q value) of the pathway enrichment. FIG. 16D: Pathway enrichment using IRAK1-specific proximal proteins. Bars represent the number of IRAK1 interacting proteins that appear in the designated pathway. Dots represent −Log(q value) of the pathway enrichment. FIG. 16E: Pathway enrichment using proximal proteins common to IRAK1 and IRAK4. Bars represent the number of interacting proteins that appear in the designated pathway. Dots represent −Log(q value) of the pathway enrichment. FIG. 16F: Interaction networks of proteins identified as IRAK4 interactors. FIG. 16G: Interaction networks of proteins identified as IRAK1 interactors. FIG. 16H: Interaction map highlighting IRAK4 interactors in the PRC2 complex and IRAK1 interactors in JAK/STAT/interferon signaling. Circle sizes indicate the adjusted P value for the identified interaction with IRAK1 or IRAK4.

FIGS. 17A-17D depict IRAK1 and IRAK4 APEX2 proximity labeling in AML cells. FIG. 17A: Immunoblots for V5, IRAK1, and phoshpo-IKKa/b in untreated versus doxycycline-treated WT THP1 transduced with empty vector and IRAK1^{KO} THP1 transduced with empty vector or inducible IRAK1-APEX2 fusion construct. For the phopsho-IKK and vinculin immunoblots, cells were treated with IL-1b (10 ng/ml). FIG. 17B: Immunoblots for V5, IRAK1, and phoshpo-IKKa/b in untreated versus doxycycline-treated WT THP1 transduced with empty vector and IRAK4^{KO} THP1 cells transduced with empty vector or the inducible IRAK4-APEX2 fusion construct. For the phopsho-IKK and vinculin immunoblots, cells were additionally treated with IL-1b (10 ng/ml). FIG. 17C: Imperial stain of biotinylated proteins pulled down with blocked-streptavidin in untreated and doxycycline-treated IRAK1^{KO} THP1 transduced with the IRAK1-APEX2 fusion construct following incubation with biotin phenol and APEX2 activation with hydrogen peroxide. FIG. 17D: Imperial stain of biotinylated proteins pulled down with blocked-streptavidin in untreated versus doxycycline-treated IRAK4^{KO} THP1 cells transduced with the IRAK4-APEX2 fusion construct following incubation with biotin phenol and APEX2 activation with hydrogen peroxide.

FIGS. 18A-18K depict that IRAK1/4 maintains undifferentiated leukemic cell states through chromatin and transcription factor networks. FIG. 18A: Heatmap of chromatin accessibility (ATAC-seq) peaks within a 3 kb distance of transcription start sites (TSS) of genes in WT, IRAK1^{KO}, IRAK4^{KO} and IRAK1/4^{dKO} THP1 cells. FIG. 18B: Total number of accessibility peaks lost and acquired in IRAK1^{KO}, MA4^{KO} and IRAK1/4^{dKO} THP1 relative to WT cells. FIG. 18C: Venn diagrams of overlap genes that are associated with both differential expression (RNA sequencing) and concordant changes in chromatin accessibility (ATAC-seq) in IRAK1^{KO}, IRAK4^{KO}, and IRAK1/4^{dKO} THP1 cells. FIGS. 18D and 18E: Heatmaps of transcription factor enrichment among genes associated with downregulation and loss of chromatin peaks (FIG. 18D) or upregulation and acquisition of open chromatin peaks (FIG. 18E) in IRAK1^{KO}, RAK4^{KO} and IRAK1/4^{dKO} THP1 cells relative to WT cells. Enrichment of transcription factor signatures was determined with the CHIP Enrichment Analysis (ChEA) 2022 library. Color intensity reflects the Log(P value) of the enrichment score. FIG. 18F: Heatmap of differential gene expression in AML patients (relative to healthy controls) using gene expression data curated from the Beat AML dataset. The heatmap represents a subset of genes that are downregulated and associated with loss of chromatin accessibility in IRAK1/4^{dKO} THP1 (“IRAK1/4 gene signature”). Unsupervised hierarchical clustering analysis resolved distinct cohorts of IRAK1/4-high signature (Group 1) and IRAK1/4-low/intermediate signature (Groups 2 and 3) AML patients. FIG. 18G: Enrichment of AML-associated mutations in IRAK1/4-high signature (Group 1) and IRAK1/4-low/intermediate signature (Groups 2 and 3) AML patients (from FIG. 18F) based on hypergeometric testing. FIG. 18H: Schematic of the CRISPR activation screen. WT and IRAK1/4^{dKO} THP1 cells were transduced with the pooled sgRNA library targeting more than 18,000 coding isoforms. After 2 weeks, deep sequencing was performed to identify candidate genes. FIG. 18I: Average MAGeCK score for candidate genes from WT and IRAK1/4^{dKO} THP1 replicate samples. Blue circles represent genes selectively enriched in IRAK1/4^{dKO} THP1 cells. FIG. 18J: Most significant transcription factors (ENCODE/ChEA analysis) selectively enriched in IRAK1/4^{dKO} THP1 cells among the top 338 candidate genes. FIG. 18K: Most significant pathways (KEGG analysis) selectively enriched in IRAK1/4^{dKO} THP1 cells among the top 338 candidate genes.

FIGS. 19A-19D depict an evaluation of the IRAK1/4 signature in MDS and AML. FIG. 19A: Heatmap showing relative expression of genes in the IRAK1/4 signaling signature in CD34+ cells from IRAK1/4-high signature (Group 1) and IRAK1/4-low/intermediate signature (Group 2) MDS patients (GSE58831). The bottom panels indicate overall survival, International Prognostic Scoring System score, and MDS subtype for each individual patient in the analysis. FIG. 19B: The heatmap showing relative expression of genes in the IRAK1/4 signaling signature in pediatric AML patients using the TARGET dataset. FIG. 19C: Percent of AML patients (from the BEAT AML dataset) diagnosed with MOS were stratified on the IRAK1/4 signature. The number in the histogram represents the number of patients with prior MOS within each group. FIG. 19D: The heatmap represents a subset of genes that are downregulated and associated with loss of chromatin accessibility in IRAK1f4dKO TH P1 (“IRAK1/4 gene signature”). Unsupervised hierar-chical clustering analysis resolved distinct cohorts of IRAK1/4-high signature (Group 1) and IRAK1/4-low/intermediate signature (Groups 2 and 3) AML patients from the TCGA dataset.

FIGS. 20A-20P depict that a dual IRAK1/4 inhibitor is more effective at suppressing MDS/AML as compared to a selective IRAK4 inhibitor. FIG. 20A: Chemical structures of Compound 14 (IRAK4-inh) and Compound 15 (IRAK1/4 dual-inh) with IC₅₀ and Ka values for IRAK1 and IRAK4. FIG. 20B: IC₅₀ curves for Compound 14 and Compound 15 in an assay measuring NF-kB activity upon TLR2 stimulation with PAM3CSK4 in THP1-Blue NF-κB SEAP reporter cells. FIG. 20C: Immunoblots for phospho-IRAK1 and total IRAK1 in THP1 and MDSL cells treated with DMSO, Compound 14 (500 nM), or Compound 15 (500 nM) for 24 hours. FIG. 20D: Heatmap of differentially expressed genes downregulated by both Compound 14 and Compound 15 (“IRAK4-dependent”), genes downregulated by Compound 15 (“IRAK1-dependent”), and genes representing the “IRAK1/4 AML signature” (from FIG. 18F). FIG. 20E: Colony formation of MDSL (250 nM), THP1 (1 μM), OCIAML3 (1 μM), AML(1714) (1 μM), AML(1294) (1 μM), AML(08) (250 nM), and MDS(3328) (250 nM) cells treated with DMSO, Compound 14, or Compound 15. FIG. 20F: Annexin V staining of MDSL, THP1, OCIAML3, AML(1714), and AML(1294) cells treated with DMSO, Compound 14 (500 nM), or Compound 15 (500 nM) for 48 hours. FIG. 20G: Representative Wright-Giemsa stains of MDSL, THP1, OCIAML3, AML(1714), and AML(1294) cells treated with DMSO, Compound 14 (500 nM), or Compound 15 (500 nM) for 12 days. FIG. 20H: Graphical illustration of IRAK1/4 signaling in LSPCs. Significance was determined with a Student's t test (*, P<0.05; **, P<0.01). Error bars represent the standard error of the mean or standard deviation. FIG. 20I: Experimental overview: AML(1714) cells derived from patients were treated in vitro vehicle (DMSO), Compound 14 (500 nM), or Compound 15 (500 nM) for 21 days. Following treatment, live cells were evaluated for colony formation and in xenografted mice. FIG. 20J: Bone marrow engraftment of AML(1714) cells in xenografted NSGS mice at day 36. Leukemic engraftment was determined as percentage of huCD45⁺huCD33⁺ cells. FIG. 20K: Kaplan Meier survival analysis of NSGS mice (n=10 mice/group) engrafted with AML(1714) cells pre-treated with the indicated inhibitors. FIG. 20L: Experimental overview: AML(64519), AML(0169), and MDS(76960) cells derived from patients were engrafted into NSGS mice. Two weeks post-engraftment, mice were randomized and treated orally (PO) daily with vehicle (PBS), Compound 14 (30 mg/kg), or Compound 15 (100 mg/kg). The concentrations were selected to equilibrate the free drug concentrations (Table 16 in the Appendix). FIGS. 20M-20O: Peripheral blood engraftment of AML(64519) (FIG. 20M), AML(0169) (FIG. 20N), and MDS(76960) (FIG. 20O) cells in xenografted NSGS mice at day 40, 29, and 48 post-treatment, respectively. Leukemic engraftment was determined as percentage of huCD45⁺huCD33⁺ cells. FIG. 20P: Kaplan Meier survival analysis of NSGS mice (n=8 mice/group) engrafted with AML(0169) cells and treated with the indicated inhibitors. Significance was determined with a Student's t test (*, P<0.05; **, P<0.01; ***, P<0.001). Error bars represent the standard error of the mean or standard deviation.

FIGS. 21A-21E depict an evaluation of the dual IRAK1/4 inhibitor in AML. FIG. 21A: Pathway enrichment using KEGG analysis of downregulated genes upon treatment with both Compound 14 and Compound 15 (IRAK4-dependent genes) as compared to vehicle control. Bar plots depict pathways ranked by −Log(P value) of enrichment score. Enriched pathways were pulled from the KEGG 2021 dataset using Enrichr. FIG. 21B: Pathway enrichment using KEGG analysis of down-regulated genes upon treatment with Compound 15 (IRAK1-dependent genes) as compared to Compound 14 and vehicle control. Bar plots depict pathways ranked by −Log(P value) of enrichment score. FIG. 21C: Myeloid and erythroid colony formation of healthy donor CD34+ cells treated with DMSO, Compound 14, or Compound 15. FIG. 21D: Immunophenotyping for CD38 expression on the indicated cells after treatment with DMSO, Compound 14, or Compound 15 for 12 days. FIG. 21E: Colony formation of AML(1714) cells treated in vitro with DMSO, Compound 14, or Compound 15 for 3 weeks. Following in vitro treatment, 250 live cells were plated in methylcellulose to assess colony formation after 10 days.

FIGS. 22A-22B depict the evaluation of IRAK1/4-deficient AML cells. FIG. 22A: Colony formation following a secondary replating of isogenic THP1 cells isolated from the primary colony plating (see FIG. 11H). FIG. 22B: Kaplan Meier survival analysis of NSGS mice (n=4 mice/group) engrafted with WT, IRAK4^{KO}, IRA1^{KO}, and IRAK1/4^{dKO} THP1 cells.

FIG. 23 depicts liver engraftment of WT (n=4), IRAK4^{KO} (n=5), IRAK1^{KO} (n=5), and IRAK1/4^{dKO} (n=5) THP1 cells in xenografted NSGS mice at time of death. Leukemic engraftment was determined as percentage of huCD45⁺huCD33⁺ cells normalized to number of days.

FIG. 24 depicts immunophenotyping of the indicated cells for CD34 expression.

FIGS. 25A-25D demonstrate that IRAK1 and IRAK4 interactomes reveal non-canonical signaling in AML. FIG. 25A: Immunoblots for IRAK1 and IRAK4 in the nuclear (Nuc) and cytoplasmic (Cyto) fractions isolated from the indicated cells. FIG. 25B: Immunoprecipitation of IRAK4 (or IgG control) followed by immunoblotting of IRAK4 and EZH2 from THP1 cells. FIG. 25C: Immunoblots for phospho-STAT5 and STAT5 in WT, IRAK4^{KO}, and IRAK1^{KO} THP1 cells. FIG. 25D: Colony formation of MyD88^{KO}, IRAK1^{KO} and IRAK4^{dKO} THP1 cells treated with DMSO or BBI608 (STAT3 inhibitor) (500 nM). Error bars represent the standard error of the mean.

FIG. 26 depicts that targeting IRAK4 results in compensation by IRAK1.

FIG. 27 depicts data demonstrating that IRAK1/4 drives NF-kB signaling.

FIG. 28 depicts that a compound of the present disclosure (Compound 8) provides enhanced survival in xenograft mice compared to emavusertib (CA-4948) and gilteritinib.

FIGS. 29A-29B depict the creation of an IRAK1/4 signature (FIG. 29A) and the analysis of the IRAK1/4 signature in patients (FIG. 29B).

### DETAILED DESCRIPTION

The following related applications are incorporated by reference herein in their entirety, and for all purposes: International Publication No. WO 2018081738, TREATMENT OF DISEASES ASSOCIATED WITH ACTIVATED IRAK, filed Oct. 30, 2017; U.S. Publication No. 2021/0292843, TREATMENT OF DISEASES ASSOCIATED WITH ACTIVATED IRAK, filed Apr. 4, 2019; International Publication No. WO 2014190163, Combination Therapy for MDS, filed May 22, 2014; U.S. Pat. No. 9,168,257, Combination Therapy for MDS, issued Oct. 27, 2015; U.S. Pat. No. 9,504,706, Combination Therapy for MDS, issued Nov. 29, 2016; U.S. Pat. No. 9,855,273, Combination Therapy for MDS, issued Jan. 2, 2018; International Publication No. WO 2018038988, Compounds, Compositions, Methods for Treating Diseases, and Methods for Preparing Compounds, filed Aug. 16, 2017; U.S. Pat. No. 11,254,667, Substituted imidazo[1,2-a]pyridines as IRAK 1/4 and FLT3 inhibitors, issued Feb. 2, 2022; U.S. Publication No. 2022/0213094, Substituted Imidazo[1,2-a]-pyridines as IRAK 1/4 and FLT3 Inhibitors, filed Jan. 4, 2022; U.S. Publication No. 2020/0199123, Substituted imidazo[1,2-a]pyridines as IRAK 1/4 and FLT3 inhibitors, filed Feb. 28, 2020; U.S. Publication No. 2022/0235042, Substituted Imidazo[1,2-a]-pyridines as IRAK 1/4 and FLT3 Inhibitors, filed Jan. 28, 2022; International Publication No. WO 2020252487, Rational therapeutic targeting of oncogenic immune signaling states in myeloid malignancies via the ubiquitin conjugating enzyme UBE2N, filed Jun. 15, 2020; International Publication No. WO 2022026935, Multi-Cyclic IRAK and FLT3 Inhibiting Compounds and Uses Thereof, filed Jul. 31, 2021; International Publication No. WO 2022140647, Multi-Cyclic IRAK and FLT3 Inhibiting Compounds and Uses Thereof, filed Dec. 23, 2021; International Publication No. WO 2023009833, Multi-Cyclic IRAK and FLT3 Inhibiting Compounds and Uses Thereof, filed Jul. 29, 2022; International Patent Application No. PCT/US2023/068520, Multi-Cyclic IRAK and FLT3 Inhibiting Compounds and Uses Thereof, filed Jun. 15, 2023, and International Patent Application No. PCT/US2023/068897, Multi-Cyclic IRAK and FLT3 Inhibiting Compounds and Uses Thereof, filed Jun. 22, 2023.

While embodiments encompassing the general disclosed concepts may take diverse forms, various embodiments will be described herein, with the understanding that the present disclosure is to be considered merely exemplary, and the general disclosed concepts are not intended to be limited to the disclosed embodiments.

Some embodiments of the disclosure include disclosed compounds. Other embodiments include compositions (e.g., pharmaceutical compositions) comprising the disclosed compound. Still other embodiments of the disclosure include compositions for treating, for example, certain diseases using the disclosed compounds. Some embodiments include methods of using the disclosed compound (e.g., in compositions or in pharmaceutical compositions) for administering and treating. Further embodiments include methods for making the disclosed compound. Yet further embodiments include methods for determining whether a particular patient is likely to be responsive to such treatment with the disclosed compounds and compositions. Unless otherwise noted, terms are to be understood according to conventional usage by those of ordinary skill in the relevant art.

The abbreviations used herein have their conventional meaning within the chemical and biological arts. The chemical structures and formulae set forth herein are constructed according to the standard rules of chemical valency known in the chemical arts. Where substituent groups are specified by their conventional chemical formulae, written from left to right, they equally encompass the chemically identical substituents that would result from writing the structure from right to left, e.g., —CH₂O— is equivalent to —OCH₂—. As used herein, in relation to compounds of Formulae (VI-5003), (I-5007), (VII-5008), (I-5009), (I-5010), (II-5010), or (III-5010), etc., the term “attached” signifies a stable covalent bond, certain preferred points of attachment being apparent to those of ordinary skill in the art.

As used herein (unless otherwise specified), the term “alkyl” means a monovalent, straight or branched hydrocarbon chain, which can be fully saturated, mono- or polyunsaturated and can include di- and multivalent radicals, having the number of carbon atoms designated (i.e., C₁-C₁₀ means one to ten carbons). For example, the terms “C₁-C₇ alkyl” or “C₁-C₄ alkyl” refer to straight- or branched-chain saturated hydrocarbon groups having from 1 to 7 (e.g., 1, 2, 3, 4, 5, 6, or 7), or 1 to 4 (e.g., 1, 2, 3, or 4), carbon atoms, respectively. Examples of C₁-C₇ alkyl groups include, but are not limited to, methyl, ethyl, n-propyl, i-propyl, n-butyl, s-butyl, t-butyl, n-pentyl, s-pentyl, n-hexyl, and n-septyl. Examples of C₁-C₄ alkyl groups include, but are not limited to, methyl, ethyl, n-propyl, i-propyl, n-butyl, s-butyl, and t-butyl.

As used herein (unless otherwise specified), the term “alkenyl” means a monovalent, straight or branched hydrocarbon chain that includes one or more (e.g., 1, 2, 3, or 4) double bonds. Double bonds can occur in any stable point along the chain and the carbon-carbon double bonds can have either the cis or trans configuration. For example, this definition shall include but is not limited to ethenyl, propenyl, butenyl, pentenyl, hexenyl, heptenyl, octenyl, nonenyl, decenyl, undecenyl, 1,5-octadienyl, 1,4,7-nonatrienyl, cyclopentenyl, cyclohexenyl, cycloheptenyl, cyclooctenyl, ethylcyclohexenyl, butenylcyclopentyl, l-pentenyl-3-cyclohexenyl, and the like. Similarly, “heteroalkenyl” refers to heteroalkyl having one or more double bonds. Further examples of alkenyl groups include, but are not limited to, vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, and 5-hexenyl.

As used herein (unless otherwise specified), the term “alkynyl” means a monovalent, straight or branched hydrocarbon chain that includes one or more (e.g., 1, 2, 3, or 4) triple bonds and that also may optionally include one or more (e.g. 1, 2, 3, or 4) double bonds in the chain. Examples of alkynyl groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl.

As used herein (unless otherwise specified), the term “alkoxy” means any of the above alkyl, alkenyl, or alkynyl groups which is attached to the remainder of the molecule by an oxygen atom (alkyl-O—). Examples of alkoxy groups include, but are not limited to, methoxy (sometimes shown as MeO—), ethoxy, isopropoxy, propoxy, and butyloxy.

The term “alkylene,” by itself or as part of another substituent, means, unless otherwise stated, a divalent radical derived from an alkyl, alkenyl, or alkynyl group, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the compounds disclosed herein. A “lower alkyl” or “lower alkylene” is a shorter chain alkyl or alkylene group, generally having eight or fewer carbon atoms.

As used herein (unless otherwise specified), the term “cycloalkyl” means a monovalent, monocyclic or bicyclic, 3, 4, 5, 6, 7, 8, 9, 10, 11, or 12 membered hydrocarbon group. The rings can be saturated or partially unsaturated. Examples of cycloalkyl groups include, but are not limited to, cyclopropyl, cyclobutyl, cyclopentyl, cyclohexyl, cycloheptyl, cyclooctyl, and bicycloalkyls (e.g., bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds). For a monocyclic cycloalkyl, the ring is not aromatic. For a bicyclic cycloalkyl, if one ring is aromatic, then the other is not aromatic. For a bicyclic cycloalkyl, one or both rings can be substituted.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms can optionally be oxidized, and the nitrogen heteroatom can optionally be quaternized. The heteroatom(s) O, N, P, S, and Si can be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH 2, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms can be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′—and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

As used herein (unless otherwise specified), the term “halogen” or “halo” means monovalent Cl, F, Br, or I. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

As used herein (unless otherwise specified), the term “aryl” means a monovalent, monocyclic or bicyclic, 5, 6, 7, 8, 9, 10, 11, or 12 member aromatic hydrocarbon group and also means polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. Examples of aryl groups include, but are not limited to, phenyl, naphthyl, tolyl, and xylyl. For an aryl that is bicyclic, one or both rings can be substituted.

As used herein (unless otherwise specified), the term “heteroaryl” means a monovalent, monocyclic or bicyclic, 5, 6, 7, 8, 9, 10, 11, or 12 membered, hydrocarbon group, where 1, 2, 3, 4, 5, or 6 carbon atoms are replaced by a hetero atom independently selected from nitrogen, oxygen, or sulfur atom, and the monocyclic or bicyclic ring system is aromatic. Heteroaryl groups (or rings) can contain from one to four heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Examples of heteroaryl groups include, but are not limited to, thienyl (or thiophenyl), furyl, indolyl, pyrrolyl, pyridinyl, pyrazinyl, oxazolyl, thiaxolyl, quinolinyl, pyrimidinyl, imidazolyl, triazolyl, tetrazolyl, 1H-pyrazol-4-yl, 1-Me-pyrazol-4-yl, pyridin-3-yl, pyridin-4-yl, 3,5-dimethylisoxazolyl, 1H-pyrrol-3-yl, 3,5-di-Me-pyrazolyl, and 1H-pyrazol-4-yl. For a bicyclic heteroaryl, if one ring is aryl, then the other is heteroaryl. For a bicyclic heteroaryl, one or both rings can have one or more hetero atoms. For a bicyclic heteroaryl, one or both rings can be substituted.

An “arylene” and a “heteroarylene,” alone or as part of another substituent, mean a divalent radical derived from an aryl and heteroaryl, respectively. Accordingly, the term “aryl” can represent an unsubstituted, mono-, di- or trisubstituted monocyclic, polycyclic, biaryl and heterocyclic aromatic groups covalently attached at any ring position capable of forming a stable covalent bond, certain preferred points of attachment being apparent to those skilled in the art (e. g. 3-indolyl, 4-imidazolyl). The aryl substituents are independently selected from the group consisting of halo, nitro, cyano, trihalomethyl, C₁₋₁₆alkyl, arylC₁₋₁₆alkyl, C₀₋₁₆alkyloxyC₀₋₁₆alkyl, arylC₀₋₁₆alkyloxyC₀₋₁₆alkyl, C₀₋₁₆alkylthioC₀₋₁₆alkyl, arylC₀₋₁₆alkylthioC₀₋₁₆alkyl, C₀-16alkylaminoC₀₋₁₆alkyl, arylC₀₋₁₆alkylaminoC₀₋₁₆alkyl, di(arylC₁₋₁₆alkyl)aminoC₀₋₁₆alkyl, C₁₋₁₆alkylCarbonylC₀₋₁₆alkyl, arylC₁₋₁₆alkylcarbonylC₀₋₁₆alkyl, C₁₋₁₆alkylcarboxyC₀₋₁₆alkyl, arylC₁₋₁₆alkylcarboxyC₀₋₁₆alkyl, C₁₋₁₆alkylcarbonylaminoC₀₋₁₆alkyl, arylC₁₋₁₆alkylcarbonylaminoC₀₋₁₆alkyl, —C₀₋₁₆alkylCOOR₄, —C₀₋₁₆alkylCONR₅R₆ wherein R₄, R₅ and R₆ are independently selected from hydrogen, C₁-C₁₁alkyl, arylC₀-C₁₁alkyl, or R₅ and R₆ are taken together with the nitrogen to which they are attached forming a cyclic system containing 3 to 8 carbon atoms with or without one C₁₋₁₆alkyl, arylC₀-C₁₆alkyl, or C₀-Cl₁₆alkylaryl substituent. Aryl includes but is not limited to pyrazolyl and triazolyl.

For brevity, the term “aryl” when used in combination with other terms (e.g., aryloxy, arylthioxy, arylalkyl) includes both aryl and heteroaryl rings as defined above. Thus, the terms “arylalkyl,” “aralkyl” and the like are meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like), or a sulfur atom. Accordingly, the terms “arylalkyl” and the like (e.g. (4-hydroxyphenyl)ethyl, (2-aminonaphthyl)hexyl, pyridylcyclopentyl) represents an aryl group as defined above attached through an alkyl group as defined above having the indicated number of carbon atoms.

The terms “cycloalkyl” and “heterocycloalkyl”, also referred to as “heterocyclyl”, by themselves or in combination with other terms, mean, unless otherwise stated, cyclic versions of “alkyl” and “heteroalkyl,” respectively. Examples of cycloalkyl include, but are not limited to, cyclopropyl, cyclobutyl, cyclopentyl, cyclohexyl, 1-cyclohexenyl, 3-cyclohexenyl, cycloheptyl, and the like. As used herein (unless otherwise specified), the term “heterocycloalkyl” or “heterocyclyl” means a monovalent, monocyclic or bicyclic, 5, 6, 7, 8, 9, 10, 11, or 12 membered, hydrocarbon, where 1, 2, 3, 4, 5, or 6 carbon atoms are replaced by a hetero atom independently selected from nitrogen atom, oxygen atom, or sulfur atom, and the monocyclic or bicyclic ring system is not aromatic. Additionally, for heterocycloalkyl, a heteroatom can occupy the position at which the heterocycle is attached to the remainder of the molecule. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, tetrahydropyran, pyrolidinyl (e.g., pyrrolidin-1-yl, pyrrolidin-2-yl, pyrrolidin-3-yl, or pyrrolidin-4-yl), piperazinyl (e.g., piperazin-1-yl, piperazin-2-yl, piperazin-3-yl, or piperazin-4-yl), piperidinyl (e.g., piperadin-1-yl, piperadin-2-yl, piperadin-3-yl, or piperadin-4-yl), and morpholinyl (e.g., morpholin-1-yl, morpholin-2-yl, morpholin-3-yl, or morpholin-4-yl,). For a bicyclic heterocyclyl, if one ring is aromatic (e.g., monocyclic aryl or heteroaryl), then the other ring is not aromatic. For a bicyclic heterocyclyl, one or both rings can have one or more hetero atoms. For a bicyclic heterocyclyl, one or both rings can be substituted and the like. A “cycloalkylene” and a “heterocycloalkylene,” alone or as part of another substituent, means a divalent radical derived from a cycloalkyl and heterocycloalkyl, respectively.

As used herein (unless otherwise specified), the term “hetero atom” means an atom selected from nitrogen atom, oxygen atom, or sulfur atom.

As used herein (unless otherwise specified), the terms “hydroxy” or “hydroxyl” means a monovalent —OH group.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ can have a specified number of carbons (e.g., “C₁-C₄ alkylsulfonyl”).

The term “carbonyloxy” represents a carbonyl group attached through an oxygen bridge. In the above definitions, the terms “alkyl” and “alkenyl” can be used interchangeably in so far as a stable chemical entity is formed, as would be apparent to those skilled in the art.

The term “linker” refers to attachment groups interposed between substituents. In some embodiments, the linker includes amido (—CONH—Rⁿ or —NHCO—Rⁿ), thioamido (—CSNH—Rⁿ or —NHCS—Rⁿ), carboxyl (—CO₂—Rⁿ or —OCORⁿ), carbonyl (—CO—Rⁿ), urea (—NHCONH—Rⁿ), thiourea (—NHCSNH—Rⁿ), sulfonamido (—NHSO₂—Rⁿ or —SO₂NH—Rⁿ), ether (—O—Rⁿ), sulfonyl (—SO₂—Rⁿ), sulfoxyl (—SO—Rⁿ), carbamoyl (—NHCO₂—Rⁿ or —OCONH—Rⁿ), or amino (—NHRⁿ) linking moieties.

Each of the above terms (e.g., “alkyl,” “heteroalkyl,” “aryl,” and “heteroaryl”, and so forth) includes both substituted and unsubstituted forms of the indicated radical. Preferred substituents for each type of radical are provided herein.

As used herein (unless otherwise specified), the term “substituted” (e.g., as in substituted alkyl) means that one or more hydrogen atoms of a chemical group (with one or more hydrogen atoms) can be replaced by one or more non-hydrogen substituents selected from the specified options. The replacement can occur at one or more positions. The term “optionally substituted” means that one or more hydrogen atoms of a chemical group (with one or more hydrogen atoms) can be, but is not required to be substituted.

A “substituent group,” as used herein, means a non-hydrogen substituent group that may be, and preferably is, a group selected from the following moieties:
(A) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(B) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from:
(i) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(ii) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from:
(a) —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl, and
(b) C₁-C₇ alkyl, C₁-C₇ heteroalkyl, C₁-C₇ perfluorinated alkyl, C₁-C₇ alkoxy, C₁-C₇ haloalkoxy, cycloalkyl, heterocycloalkyl, aryl, and heteroaryl, substituted with at least one substituent selected from: —NH₂, —SH, —CN, —CF₃, —NO₂, halogen, hydroxy, oxo, —CN, methanoyl (—COH), carboxy (—CO₂H), nitro (—NO₂), —N(CH₃)₂, ethynyl (—CCH), propynyl, sulfo (—SO₃H), CONH₂, —CON(CH₃)₂, unsubstituted C₁-C₇ alkyl, unsubstituted C₁-C₇ heteroalkyl, unsubstituted C₁-C₇ perfluorinated alkyl, unsubstituted C₁-C₇ alkoxy, unsubstituted C₁-C₇ haloalkoxy, unsubstituted cycloalkyl, unsubstituted heterocycloalkyl, unsubstituted aryl, unsubstituted heteroaryl.

A “size-limited substituent” or “size-limited substituent group,” as used herein, means a group, e.g., selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2-20-membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4-8-membered heterocycloalkyl.

A “lower substituent” or “lower substituent group,” as used herein, means a group, e.g., selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2-8-membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5-7-membered heterocycloalkyl.

The term “about” used in the context of a numeric value indicates a range of +/−10% of the numeric value, unless expressly indicated otherwise.

Some compounds of the disclosure can have one or more chiral centers and can exist in and be isolated in optically active and racemic forms, for any of the one or more chiral centers.

Some compounds can exhibit polymorphism. The compounds of the present disclosure (e.g., Formula I) encompass any optically active, racemate, stereoisomer form, polymorphism, or mixtures thereof. If a chiral center does not provide an indication of its configuration (i.e., R or S) in a chemical structure, it should be considered to represent R, S or a racemate.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or microdissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing,” “analyzing,” and “assaying” can be used interchangeably and can include quantitative and/or qualitative determinations.

As used herein, the term “monitoring” with reference to a type of cancer refers to a method or process of determining the severity or degree of the type of cancer or stratifying the type of cancer based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to survival/mortality over a given time horizon. For example, “outcome” can refer to survival/mortality over 1 month, 3 months, 6 months, 1 year, 5 years, or 10 years or longer. In some embodiments, an increased risk for a poor outcome indicates that a therapy has had a poor efficacy, and a reduced risk for a poor outcome indicates that a therapy has had a good efficacy.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any suitable (e.g., treatable) member of the animal kingdom. In the methods, the subject is preferably a mammal. In the methods, the subject is preferably a human patient. In the methods, the subject may be a mammalian pediatric patient. In the methods, the pediatric patient is a mammalian (e.g., preferably human) patient under 18 years of age, while an adult patient is 18 or older.

As used herein, the term “treating” (and its variations, such as “treatment” “treating,” “treat,” and the like) is, unless stated otherwise, to be considered in its broadest context and refers to obtaining a desired pharmacologic and/or physiologic effect. In particular, for example, the term “treating” may not necessarily imply or require that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. In some aspects, “treating” may not require or include prevention. As used herein, reference to “treating” an animal includes but is not limited to prophylactic treatment and therapeutic treatment. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, preferably in a mammal (e.g., in a human), and may include one or more of: (a) preventing the disease from occurring in a subject which may be predisposed to the disease but has not yet been diagnosed as having it; (b) inhibiting the disease, i.e., arresting its development; and (c) relieving the disease, i.e., causing regression or elimination of the disease and/or relieving one or more disease symptoms. In particular aspects of the methods, such as conditions or disorders characterized by dysregulated IRAK expression or dysregulated (e.g., hyperactive) IRAK-mediated signaling pathway(s), treatment may be or include reducing such expression or signaling. “Treatment” can also encompass delivery of an agent or administration of a therapy in order to provide for a pharmacologic effect, even in the absence of a disease or condition. Any of the compositions (e.g., pharmaceutical compositions) described herein can be used to treat a suitable subject.

“Therapeutically effective amount” means an amount effective to achieve a desired and/or beneficial effect. An effective amount can be administered in one or more administrations. In the methods, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of tumor size or blood cell count, or any other suitable measurement.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, an mRNA “isoform” is an alternative transcript for a specific mRNA or gene. This term includes pre-mRNA, immature mRNA, mature mRNA, cleaved or otherwise truncated, shortened, or aberrant mRNA, modified mRNA (e.g. containing any residue modifications, capping variants, polyadenylation variants, etc.), and the like.

“Antibody” or “antibody peptide(s)” refer to an intact antibody, or a binding fragment thereof that competes with the intact antibody for specific binding; this definition also encompasses monoclonal and polyclonal antibodies. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody, for example, substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

Embodiments of the disclosure set forth herein include disclosed compounds (e.g., compounds of Formulas (VI-5003), (1-5007), (VII-5008), (1-5009), (1-5010), (II-5010), or (III-5010)). Other embodiments include compositions (e.g., pharmaceutical compositions) comprising the disclosed compound. Still other embodiments of the disclosure include compositions (e.g., pharmaceutical compositions) for treating, for example, certain diseases using the disclosed compounds. Some embodiments include methods of using the disclosed compound (e.g., in compositions or in pharmaceutical compositions) for administering and treating (e.g., diseases such as cancer or blood disorders). Some embodiments include methods of determining whether a patient is suitable for, or likely to respond favorably to, a particular treatment. Further embodiments include methods for making the disclosed compounds.

Additional embodiments of the disclosure are also discussed herein.

### Compounds

In one aspect, the present disclosure provides a compound which inhibits RAK1 and IRAK4. In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the RAK4:IRAK1 potency ratio is calculated from the IRAK1 and IRAK4 IC₅₀ measurements.

In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits IRAK1, IRAK4, and FLT3.

In one aspect, the present disclosure provides a compound of Formula (I-5007)

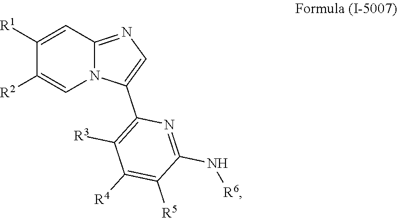

Formula (1-5007), or a salt, ester, solvate, optical isomer, geometric isomer, salt of an isomer, prodrug, or derivative thereof, wherein:

R¹ is selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, —C(═O)NR^{31a}R^{31b}, cycloalkyl, spiro-fused cycloalkyl, heterocyclyl, aryl, heteroaryl, or fused ring heteroaryl, which methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R² is selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, which methanoyl (—COH), carboxy (—CO₂H), C₂-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R³, R⁴, and R⁵ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, —O-aryl, aryl, heteroaryl, or fused ring heteroaryl, which methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, —O-aryl, aryl, heteroaryl, or fused ring heteroaryl is optionally substituted with one or more of halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R⁶ is selected from:

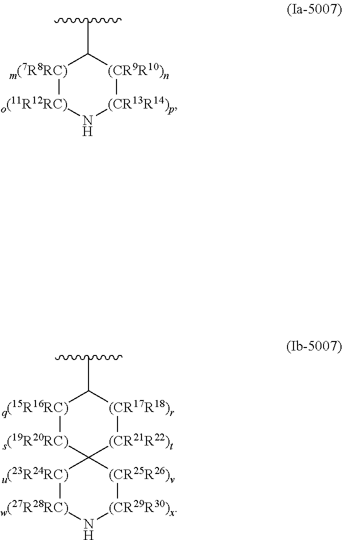

or C₃-C₆ cycloalkyl substituted with one or more —NR³³R³⁴;
R⁷, R⁸, R⁹, R¹⁰, R¹¹, R¹², R¹³, R¹⁴ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, which methanoyl (—COH), carboxy (—CO₂H), C₁-C₇ alkyl, C₂-C₇ alkenyl, C₂-C₇ alkynyl, C₂-C₆ alkoxy, cycloalkyl, spiro-fused cycloalkyl, heterocyclyl, aryl, heteroaryl, or fused ring heteroaryl is optionally substituted with one or more halogen;
R¹⁵, R¹⁶, R¹⁷, R¹⁸, R¹⁹, R²⁰, R²¹, R²², R²³, R²⁴, R²⁵, R²⁶, R²⁷, R²⁸, R²⁹, and R³⁰ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, which methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; R^{31a} and R^{31b} are each independently selected from H, C₁-C₆ alkyl, —(CH₂)ₐ—(C₃-C₆ cycloalkyl), —(CH₂)_{b}—C₂-C₆ heterocyclyl, —(CH₂)_{c}—C₃-C₉ heteroaryl, and —(C₁-C₆ alkyl)-O—(C₁-C₆ alkyl), wherein the C₁-C₆ alkyl, C₃-C₇ cycloalkyl, C₂-C₆ heterocyclyl, and C₃-C₉ heteroaryl are each independently optionally substituted with one or more of halogen, hydroxy, oxo, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R³³ and R³⁴ are each independently selected from H and C₁-C₆ alkyl;
a, b, and c are each independently selected from 0, 1, 2, 3, 4, 5, or 6; and
m, n, o, p, q, r, s, t, u, v, w, and x are each independently selected from 0, 1, 2, 3, 4, or 5,
where q+r+s+t is at least 1, and where u+v+w+x is at least 1.

In an embodiment, the compound of Formula (I-5007) is a compound of Formula (IIf-5007):

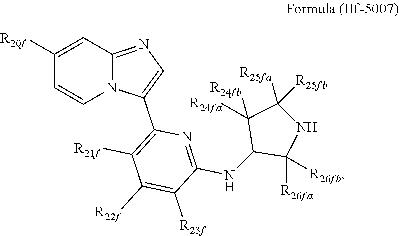

or a salt, ester, solvate, optical isomer, geometric isomer, or salt of an isomer thereof, wherein:
R_{2of} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR_{27fa}R_{27fb}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{21f}, R₂₂ᵣ, and R_{23f} are each independently selected from H and halogen; R_{24fa}, R_{24f}, R_{25fa}, R_{25fb}, R_{26fa}, and R_{26fb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
R_{27fa} and R_{27f} are each independently selected from H, C₁-C₆ alkyl, and C₃-C₆ cycloalkyl, wherein C₁-C₆ alkyl and C₃-C₆ cycloalkyl are each optionally substituted with one or more halogen.

In an embodiment, one or more of R_{24fa}, R_{24fb}, R_{25fa}, R_{25fb}, R_{26fa}, and R_{26fb} is independently selected from halogen, —OH, optionally substituted C₁-C₆ alkyl, and optionally substituted C₁-C₆ alkoxy. In another embodiment, each of R_{24fa}, R_{24fb}, R_{25fa}, R_{25fb}, R_{26fa}, and R_{26fb} is H.

In an embodiment, the compound of Formula (I-5007) is a compound of Formula (IIg-5007):

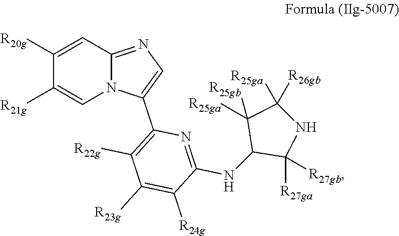

or a salt, ester, solvate, optical isomer, geometric isomer, or salt of an isomer thereof; wherein: R_{20g} is selected from H, C₁-C₆ alkoxy, imidazolyl, triazolyl, and —C(═O)NR_{29ga}R_{29gb}, wherein C₁-C₆ alkoxy is optionally substituted with one or more halogen atoms;
R_{21g} is selected from halogen, C₁-C₆ alkyl, C₁-C₆ alkoxy, C₃-C₆ cycloalkyl,

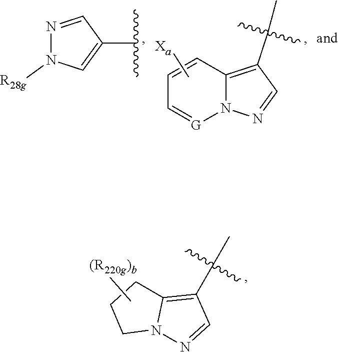

wherein C₁-C₆ alkyl and C₁-C₆ alkoxy are each optionally substituted with one or more substituents selected from —OH and halogen, and C₃-C₆ cycloalkyl is optionally substituted with one or more substituents selected from C₁-C₆ alkyl and halogen;
R_{22g}, R_{23g}, and R_{24g} are each independently selected from H and halogen;
R_{25ga}, R_{25gb}, R_{26ga}, R_{26gb}, R_{27ga}, and R_{27gb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms;
R_{28g} is selected from H, C₁-C₆ alkyl, and —(CH₂)_{d}—(C₃-C₆ cycloalkyl), wherein C₁-C₆ alkyl and —(CH₂)_{d}—(C₃-C₆ cycloalkyl) are each optionally substituted with one or more substituents selected from —OH and halogen;
R_{29ga} and R_{29gb} are each independently selected from H, C₁-C₆ alkyl, and C₃-C₆ cycloalkyl, wherein C₁-C₆ alkyl and C₃-C₆ cycloalkyl are each optionally substituted with one or more halogen;
each R_{220g} is independently C₁-C₆ alkyl;
G is N or CH;
X is halogen;
a is 0, 1, 2, or 3;
b is 0, 1, 2, 3, 4, 5, or 6; and
d is 0, 1, 2, or 3.

In an embodiment, one or more of R_{25g}a, R_{25g}b, R_{26g}a, R_{26g}b, R_{27g}a, and R_{27g}b is independently selected from halogen, —OH, optionally substituted C₁-C₆ alkyl, and optionally substituted C₁-C₆ alkoxy. In another embodiment, each of R_{25g}a, R_{25g}b, R_{26g}a, R_{26g}b, R_{27ga}, and R_{27gb} is H.

In an embodiment, the compound of Formula (1-5007) is a compound of Formula (IIh-5007):

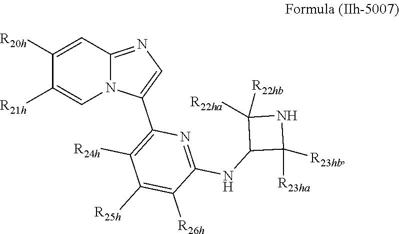

or a salt, ester, solvate, optical isomer, geometric isomer, or salt of an isomer thereof, wherein:
R₂₀ₕ is selected from H, C₁-C₆ alkoxy, imidazolyl, triazolyl, and —C(═O)NR₂₇ₕₐR_{27bb};
R₂₁ₕ is selected from C₁-C₆ alkyl and C₃-C₆ cycloalkyl, wherein C₁-C₆ alkyl and C₃-C₆ cycloalkyl are each optionally substituted with one or more substituents selected from —OH and halogen;
R₂₂ₕₐ, R_{22hb}, R₂₃ₕₐ, and R_{23hb} are each independently selected from H and C₁-C₆ alkyl,
wherein C₁-C₆ alkyl is optionally substituted with one or more halogen atoms;
R₂₄ₕ, R₂₅ₕ, and R₂₆ₕ are each independently selected from H and halogen; and R₂₇ₕₐ and R_{27hb} are each independently selected from H, C₁-C₆ alkyl, and C₃-C₆ cycloalkyl, wherein C₁-C₆ alkyl and C₃-C₆ cycloalkyl are each optionally substituted with one or more halogen.

In an embodiment, one or more of R₂₂ₕₐ, R_{22hb}, R₂₃ₕₐ, and R_{23hb} is independently optionally substituted C₁-C₆ alkyl. In another embodiment, each of R₂₂ₕₐ, R_{22hb}, R₂₃ₕₐ, and R_{23hb} is H.

In an embodiment, the compound of Formula (1-5007) is a compound of Formula (IIi-5007):

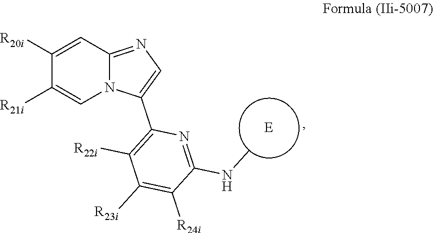

or a salt, ester, solvate, optical isomer, geometric isomer, or salt of an isomer thereof; wherein:

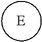

is selected from

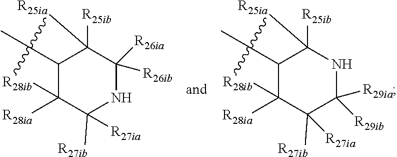

R₂₀ᵢ is selected from H, —O—(C₃-C₆ cycloalkyl), C₁-C₆ alkoxy, imidazolyl, triazolyl, and —C(═O)NR₂₂ᵢₐR_{221ib}, wherein C₁-C₆ alkoxy is optionally substituted with one or more halogen atoms;
R₂₁ᵢ is selected from halogen, C₁-C₆ alkyl, C₁-C₆ alkoxy, C₃-C₆ cycloalkyl, and

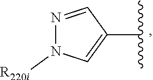

wherein C₁-C₆ alkyl and C₁-C₆ alkoxy are each optionally substituted by one or more substituents selected from halogen and —OH, and C₃-C₆ cycloalkyl is optionally substituted by one or more substituents selected from OH and halogen;
R₂₂ᵢ, R₂₃ᵢ, and R₂₄ᵢ are each independently selected from H and halogen;
R₂₅ᵢₐ, R_{25ib}, R₂₆ᵢₐ, R_{26ib}, R₂₇ᵢₐ, R_{27ib}, R₂₈ᵢₐ, R_{28ib}, R₂₉ᵢₐ, and R_{29ib} are each independently selected from H, halogen, —OH, or C₁-C₆ alkyl;
R₂₂₀ᵢ is selected from H, C₁-C₆ alkyl, and —(CH₂)ₑ—(C₃-C₆ cycloalkyl), wherein C₁-C₆ alkyl and —(CH₂)ₑ—(C₃-C₆ cycloalkyl) are each optionally substituted with one or more substituents selected from OH and halogen;
R₂₂₁ᵢₐ and R_{221ib} are each independently selected from H, C₁-C₆ alkyl, and C₃-C₆ cycloalkyl, wherein C₁-C₆ alkyl and C₃-C₆ cycloalkyl are each optionally substituted with one or more halogen; and
e is 0, 1, 2, or 3.

In an embodiment, one or more of R₂₅ᵢₐ, R_{25ib}, R₂₆ᵢₐ, R_{26ib}, R₂₇ᵢₐ, R_{27ib}, R₂₈ᵢₐ, R_{28ib}, R₂₉ᵢₐ, and R_{29ib} is independently selected from halogen, —OH, and C₁-C₆ alkyl. In another embodiment, each of R₂₅ᵢₐ, R_{25ib}, R₂₆ᵢₐ, R_{26ib}, R₂₇ᵢₐ, R_{27ib}, R₂₈ᵢₐ, R_{28ib}, R₂₉ᵢₐ, and R_{29ib} is H.

In an embodiment, the compound of Formula (II-5007) is a compound of Formula (IIj-5007):

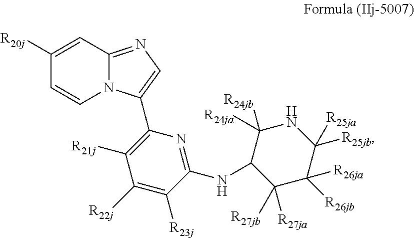

or a salt, ester, solvate, optical isomer, geometric isomer, or salt of an isomer thereof; wherein:
R₂₀ⱼ is selected from C₁-C₆ alkoxy, —O—(C₃-C₆ cycloalkyl), imidazolyl, triazolyl, and —C(═O)NR₂₈ⱼₐR_{28jb}, wherein C₁-C₆ alkoxy is optionally substituted with one or more halogen substituents;
R₂₁ⱼ, R₂₂ⱼ, and R₂₃ⱼ are each independently selected from H and halogen;
R₂₄ⱼₐ, R_{24jb}, R₂₅ⱼₐ, R_{25jb}, R₂₆ⱼₐ, R_{26jb}, R₂₇ⱼₐ, and R_{27jb} are each independently selected from H, halogen, —OH, and C₁-C₆ alkyl; and
R₂₈ⱼₐ and R_{25jb} are each independently selected from H, C₁-C₆ alkyl, and C₃-C₆ cycloalkyl, wherein C₁-C₆ alkyl and C₃-C₆ cycloalkyl are each optionally substituted with one or more halogen.

In an embodiment, one or more of R₂₄ⱼₐ, R_{24jb}, R₂₅ⱼₐ, R_{25jb}, R₂₆ⱼₐ, R_{26jb}, R₂₇ⱼₐ, and R_{27jb} is selected from halogen, —OH, and C₁-C₆ alkyl. In another embodiment, each of R₂₄ⱼₐ, R_{24jb}, R₂₅ⱼₐ, R_{25jb}, R₂₆ⱼₐ, R_{26jb}, R₂₇ⱼₐ, and R_{27jb} is H.

In an embodiment, the compound of Formula (I-5007) is a compound of Formula (IIk-5007):

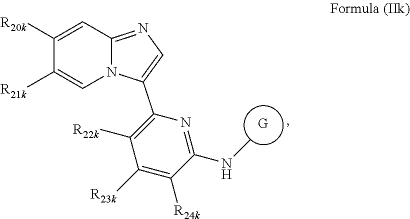

or a salt, ester, solvate, optical isomer, geometric isomer, or salt of an isomer thereof; wherein:

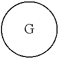

is selected from

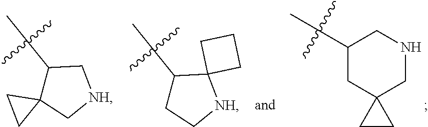 [SMILES: *C1CNCC12CC2 ; *C1CCNC12CCC2 ; *C1CNCC2(CC2)C1]

R₂₀ₖ is selected from H, C₁-C₆ alkoxy, imidazolyl, triazolyl, and —C(═O)R₂₅ₖₐR_{25kb}, wherein C₁-C₆ alkoxy is optionally substituted with one or more halogen atoms;

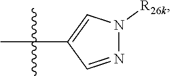

R₂₁ₖ is selected from H, C₁-C₆ alkyl, C₁-C₆ alkoxy, C₃-C₆ cycloalkyl, and N wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen;
R₂₂ₖ, R₂₃ₖ, and R₂₄ₖ are each independently selected from H and halogen; R₂₅ₖₐ and R_{25kb} are each independently selected from H, C₁-C₆ alkyl, and C₃-C₆ cycloalkyl, wherein C₁-C₆ alkyl and C₃-C₆ cycloalkyl are each optionally substituted with one or more halogen; and
R₂₆ₖ is selected from H, C₁-C₆ alkyl, and C₃-C₆ cycloalkyl, wherein C₁-C₆ alkyl and C₃-C₆ cycloalkyl are each optionally substituted with one or more substituents selected from halogen and —OH.

In one embodiment, the compound of Formula (1-5007) is a compound of Formula (IIm-5007):

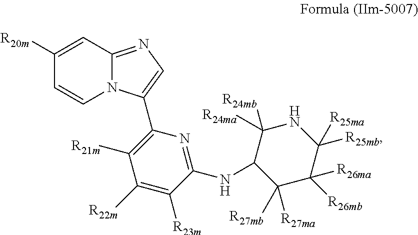

or a salt, ester, solvate, optical isomer, geometric isomer, or salt of an isomer thereof; wherein:
R₂₀ₘ is selected from C₁-C₆ alkyl, C₁-C₆ alkoxy, —O—(C₃-C₆ cycloalkyl), C₃-C₉ heteroaryl, and —C(═O)NHR₂₈ₘ, wherein C₁-C₆ alkyl is optionally substituted with one or more substituents selected from halogen and —OH, wherein C₁-C₆ alkoxy is optionally substituted with one or more halogen, and wherein C₃-C₉ heteroaryl is optionally substituted with one or more C₁-C₆ alkyl;
R₂₁ₘ, R₂₂ₘ, and R₂₃ₘ are each independently selected from H and halogen;
R₂₄ₘₐ, R_{24mb}, R₂₅ₘₐ, R_{25mb}, R₂₆ₘₐ, R_{26mb}, R₂₇ₘₐ, and R_{27mb} are each independently selected from H and halogen, wherein at least one of R₂₄ₘₐ, R_{24mb}, R₂₅ₘₐ, R_{25mb}, R₂₆ₘₐ, R_{26mb}, R₂₇ₘₐ, and R_{27mb} is halogen;
R₂₈ₘ is selected from H, C₁-C₆ alkyl, —(C₁-C₆ alkyl)-O—(C₁-C₆ alkyl), —(CH₂)ₙ—C₃-C₆ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (I-5007) is a compound of Formula (IIn-5007):

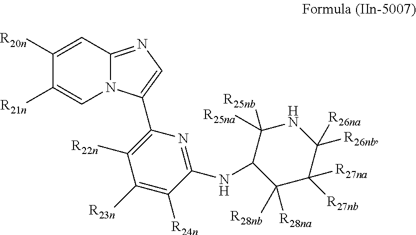

or a salt, ester, solvate, optical isomer, geometric isomer, or salt of an isomer thereof, wherein:
R₂₀ₙ is selected from H, C₁-C₆ alkyl, C₁-C₆ alkoxy, and —C(═O)NHR₂₂₀ₙ;
R₂₁ₙ is selected from C₁-C₆ alkyl and C₂-C₆ heterocyclyl, wherein the C₁-C₆ is optionally substituted with one or more substituents selected from —OH and halogen;
R₂₂ₙ, R₂₃ₙ, and R₂₄ₙ are each independently selected from H and halogen;
R₂₅ₙₐ, R_{25nb}, R₂₆ₙₐ, R_{26nb}, R₂₇ₙₐ, R_{27nb}, R₂₈ₙₐ, and R_{28nb} are each independently selected from H and halogen, wherein at least one of R₂₅ₙₐ, R_{25nb}, R₂₆ₙₐ, R_{26nb}, R₂₇ₙₐ, R_{27nb}, R₂₉ₙₐ, and R_{29nb} is halogen;
R₂₂₀ₙ is selected from H, C₁-C₆ alkyl, —(C₁-C₆ alkyl)-O—(C₁-C₆ alkyl), —(CH₂)ₙ—C₃-C₆ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (1-5007) is a compound of Formula (IIo-5007):

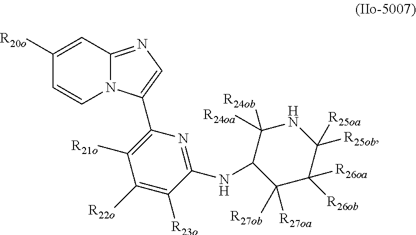

or a salt, ester, solvate, optical isomer, geometric isomer, or salt of an isomer thereof; wherein:
R₂₀ₒ is selected from C₁-C₆ alkyl, C₁-C₆ alkoxy, —O—(C₃-C₆ cycloalkyl), C₃-C₉ heteroaryl, and —C(═O)NHR₂₈ₒ, wherein C₁-C₆ alkyl is optionally substituted with one or more substituents selected from halogen and —OH, wherein C₁-C₆ alkoxy is optionally substituted with one or more halogen, and wherein C₃-C₉ heteroaryl is optionally substituted with one or more substituents selected from C₁-C₆ alkyl and halogen;
R₂₁ₒ, R₂₂ₒ, and R₂₃ₒ are each independently selected from H and halogen;
R₂₄ₒₐ, R_{24ob}, R₂₅ₒₐ, R_{25ob}, R₂₆ₒₐ, R_{26ob}, R₂₇ₒₐ, and R_{27ob} are each H;
R₂₈ₒ is selected from H, C₁-C₆ alkyl, —(C₁-C₆ alkyl)-O—(C₁-C₆ alkyl), —(CH₂)ₙ—C₃-C₇ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (1-5007) is a compound of Formula (IIp-5007):

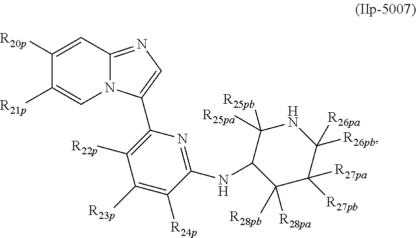

or a salt, ester, solvate, optical isomer, geometric isomer, or salt of an isomer thereof; wherein:
R₂₀ₚ is selected from H, C₁-C₆ alkyl, C₁-C₆ alkoxy, and —C(═O)NHR₂₂₀ₚ;
R₂₁ₚ is selected from C₁-C₆ alkyl, C₁-C₆ alkoxy, and C₂-C₆ heterocyclyl, wherein the C₁-C₆ is optionally substituted with one or more substituents selected from —OH and halogen;
R₂₂ₚ, R₂₃ₚ, and R₂₄ₚ are each independently selected from H and halogen;
R₂₅ₚₐ, R_{25pb}, R₂₆ₚₐ, R_{26pb}, R₂₇ₚₐ, R_{27pb}, R₂₈ₚₐ, and R_{28pb} are each H;
R₂₂₀ₚ is selected from H, C₁-C₆ alkyl, —(C₁-C₆ alkyl)-O—(C₁-C₆ alkyl), —(CH₂)ₙ—C₃-C₆ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (1-5007) is a compound of Formula (IIq-5007):

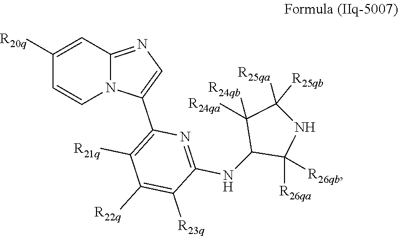

or a salt, ester, solvate, optical isomer, geometric isomer, or salt of an isomer thereof; wherein:
R_{20q} is selected from C₁-C₆ alkyl, C₁-C₆ alkoxy, —O—(C₃-C₆ cycloalkyl), C₃-C₉ heteroaryl, and —C(═O)NHR_{28q}, wherein C₁-C₆ alkyl is optionally substituted with one or more substituents selected from halogen and —OH, wherein C₁-C₆ alkoxy is optionally substituted with one or more halogen, and wherein C₃-C₉ heteroaryl is optionally substituted with one or more C₁-C₆ alkyl;
R_{21q}, R_{22q}, and R_{23q} are each independently selected from H and halogen;
R_{24qa}, R_{24qb}, R_{25qa}, R_{25gb}, R_{26qa}, and R_{26qb} are each independently selected from H and halogen, wherein at least one of R_{24qa}, R_{24qb}, R_{25qa}, R_{25qb}, R_{26qa}, and R_{26qb} is halogen;
R_{28q} is selected from H, C₁-C₆ alkyl, —(C₁-C₆ alkyl)-O—(C₁-C₆ alkyl), —(CH₂)ₙ—C₃-C₇ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (1-5007) is a compound of Formula (IIr-5007):

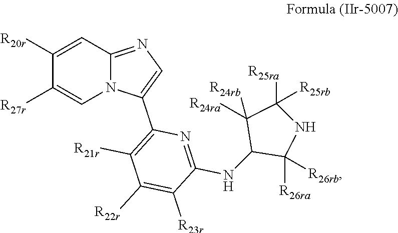

or a salt, ester, solvate, optical isomer, geometric isomer, or salt of an isomer thereof; wherein:
R₂₀ᵣ is selected from H, C₁-C₆ alkyl, C₁-C₆ alkoxy, and —(C═O)NHR₂₈ᵣ;
R₂₁ᵣ, R₂₂ᵣ, and R₂₃ᵣ are each independently selected from H and halogen;
R₂₄ᵣₐ, R_{24rb}, R₂₅ᵣₐ, R_{25rb}, R₂₆ᵣₐ, and R_{26rb} are each independently selected from H and halogen, wherein one or more of R₂₄ᵣₐ, R_{24rb}, R₂₅ᵣₐ, R_{25rb}, R₂₆ᵣₐ, and R_{26rb} is halogen;
R₂₇ᵣ is selected from C₁-C₆ alkyl and C₂-C₆ heterocyclyl, wherein the C₁-C₆ is optionally substituted with one or more substituents selected from —OH and halogen;
R₂₈ᵣ is selected from H, C₁-C₆ alkyl, —(C₁-C₆ alkyl)-O—(C₁-C₆ alkyl), —(CH₂)ₙ—C₃-C₇ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (I-5007) is a compound of Formula (IIs-5007):

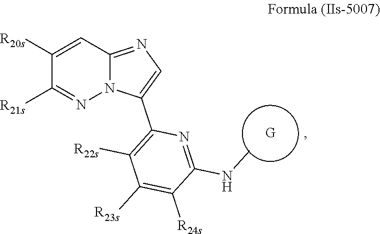

or a salt, ester, solvate, optical isomer, geometric isomer, or salt of an isomer thereof; wherein:

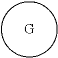

is selected from

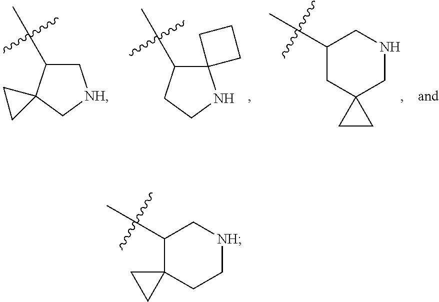 [SMILES: *C1CNCC12CC2 ; *C1CCNC12CCC2 ; *C1CNCC2(CC2)C1 ; *C1CNCCC12CC2]

R₂₀ₛ is selected from H, C₁-C₆ alkyl, C₁-C₆ alkoxy, —O—(C₃-C₆ cycloalkyl), C₃-C₉ heteroaryl, and —C(═O)NHR₂₈ₛ, wherein C₁-C₆ alkyl is optionally substituted with one or more substituents selected from halogen and —OH, wherein C₁-C₆ alkoxy is optionally substituted with one or more halogen, and wherein C₃-C₉ heteroaryl is optionally substituted with one or more C₁-C₆ alkyl;
R₂₁ₛ is selected from H, C₁-C₆ alkyl and C₂-C₆ heterocyclyl, wherein the C₁-C₆ is optionally substituted with one or more substituents selected from —OH and halogen;
R₂₂ₛ, R₂₃ₛ, and R₂₄ₛ are each independently selected from H and halogen;
R₂₈ₛ is selected from H, C₁-C₆ alkyl, —(C₁-C₆ alkyl)-O—(C₁-C₆ alkyl), —(CH₂)ₙ—C₃-C₇ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In one embodiment, the compound of Formula (1-5007) is a compound of Formula (IIt-5007):

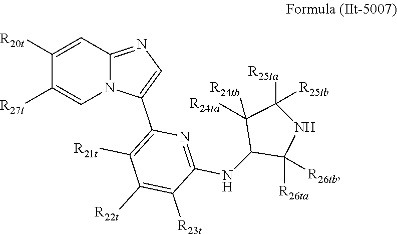

or a salt, ester, solvate, optical isomer, geometric isomer, or salt of an isomer thereof; wherein:
R₂₀ₜ is selected from H, C₁-C₆ alkyl, C₁-C₆ alkoxy, and —(C═O)NHR₂₈ₜ;
R₂₁ₜ, R₂₂ₜ, and R₂₃ₜ are each independently selected from H and halogen;
R₂₄ₜₐ, R_{24tb}, R₂₅ₜₐ, R_{25tb}, R₂₆ₜₐ, and R_{26tb} are each independently H;
R₂₇ₜ is selected from C₁-C₆ alkyl, C₃-C₆ cycloalkyl, and C₂-C₆ heterocyclyl, wherein the C₁-C₆ is optionally substituted with one or more substituents selected from —OH and halogen and the C₃-C₆ cycloalkyl is optionally substituted by one or more substituents selected from C₁-C₆ alkyl, OH, and halogen;
R₂₈ₜ is selected from H, C₁-C₆ alkyl, —(C₁-C₆ alkyl)-O—(C₁-C₆ alkyl), —(CH₂)ₙ—C₃-C₇ cycloalkyl, and —(CH₂)ₙ—C₂-C₆ heterocyclyl, wherein the C₁-C₆ alkyl is optionally substituted with one or more substituents selected from —OH and halogen, and wherein the C₃-C₆ cycloalkyl and C₂-C₆ heterocyclyl are each independently optionally substituted with one or more substituents selected from C₁-C₆ alkyl and halogen; and
each n is independently 0, 1, 2, 3, 4, 5, or 6.

In an embodiment, the compound of Formula (1-5007) is a compound of Formula (IIq-5007):

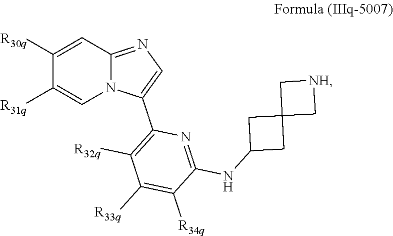

or a salt, ester, solvate, optical isomer, geometric isomer, or salt of an isomer thereof; wherein:
R_{30q} is selected from H, C₁-C₆ alkoxy, imidazolyl, triazolyl, and —C(═O)NR_{35qa}R_{35qb};

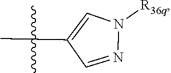

R_{31q} is selected from C₁-C₆ alkyl, C₁-C₆ alkoxy, C₃-C₆ cycloalkyl, and N wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen;
R_{32q}, R_{33q}, and R_{34q} are each independently selected from H and halogen;
R_{35qa} and R_{35qb} are each independently selected from H, C₁-C₆ alkyl, and C₃-C₆ cycloalkyl, wherein C₁-C₆ alkyl and C₃-C₆ cycloalkyl are each optionally substituted with one or more halogen; and
R_{36q} is selected from H, C₁-C₆ alkyl, and C₃-C₆ cycloalkyl, wherein C₁-C₆ alkyl and C₃-C₆ cycloalkyl are each optionally independently substituted with one or more substituents selected from halogen and —OH.

In an embodiment, the compound of Formula (1-5007) is a compound of Formula (IIIr-5007):

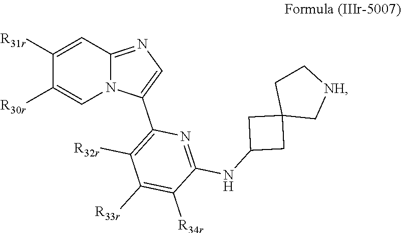

or a salt, ester, solvate, optical isomer, geometric isomer, or salt of an isomer thereof; wherein:

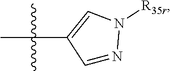

R₃₀ᵣ is selected from C₁-C₆ alkyl, C₁-C₆ alkoxy, C₃-C₆ cycloalkyl, and N wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen;
R₃₁ᵣ is selected from H, imidazolyl, triazolyl, and —C(═O)NR₃₆ₘR_{36rb};
R₃₂ᵣ, R₃₃ᵣ, and R₃₄ᵣ are each independently selected from H and halogen;
R₃₅ᵣ is selected from H, C₁-C₆ alkyl, and C₃-C₆ cycloalkyl, wherein C₁-C₆ alkyl and C₃-C₆ cycloalkyl are each optionally substituted with one or more substituents selected from halogen and —OH; and
R₃₆ᵣₐ and R_{36rb} are each independently selected from H, C₁-C₆ alkyl, and C₃-C₆ cycloalkyl, wherein C₁-C₆ alkyl and C₃-C₆ cycloalkyl are each optionally substituted with one or more halogen.

In an embodiment, the compound of Formula (1-5007) is a compound of Formula (Ills-5007):

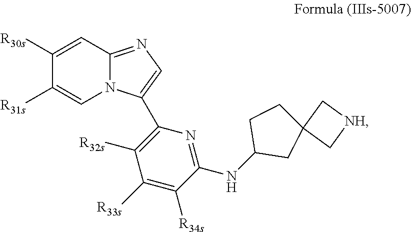

or a salt, ester, solvate, optical isomer, geometric isomer, or salt of an isomer thereof, wherein:
R₃₀ₛ is selected from H, C₁-C₆ alkoxy, imidazolyl, triazolyl, and —C(═O)NR₃₅ₛₐR_{35sb};

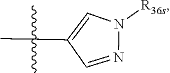

R₃₁ₛ is selected from C₁-C₆ alkyl, C₁-C₆ alkoxy, C₃-C₆ cycloalkyl, and N wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen;
R₃₂ₛ, R₃₃ₛ, and R₃₄ₛ are each independently selected from H and halogen;
R₃₅ₛₐ and R₃₅ss are each independently selected from H, C₁-C₆ alkyl, and C₃-C₆ cycloalkyl, wherein C₁-C₆ alkyl and C₃-C₆ cycloalkyl are each optionally substituted with one or more halogen; and
R₃₆ₛ is selected from H, C₁-C₆ alkyl, and C₃-C₆ cycloalkyl, wherein C₁-C₆ alkyl and C₃-C₆ cycloalkyl are each optionally substituted with one or more substituents selected from halogen and —OH.

In an embodiment, R₆ of Formula (1-5007) is C₃-C₆ cycloalkyl substituted with one or more —NR₃₃R₃₄ and the compound of Formula (I-5007) is a compound of Formula (IV-5007):

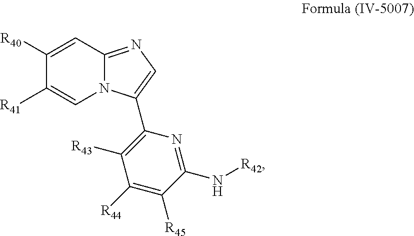

or a salt, ester, solvate, optical isomer, geometric isomer, or salt of an isomer thereof; wherein:
R₄₀ is selected from H, C₁-C₆ alkoxy, imidazolyl, triazolyl, and —C(═O)NR₄₆ₐR_{46b}, wherein C₁-C₆ alkoxy is optionally substituted with one or more halogen atoms;
R₄₁ is selected from C₁-C₆ alkyl, C₁-C₆ alkoxy, C₃-C₆ cycloalkyl, and

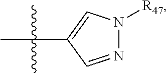

wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen;
R₄₂ is C₃-C₆ cycloalkyl substituted with one or more —NR₄₈ₐR_{48b};
R₄₃, R₄₄, and R₄₅ are each independently selected from H and halogen;
R₄₆ₐ and R_{46b} are each independently selected from H, C₁-C₆ alkyl, and C₃-C₆ cycloalkyl, wherein C₁-C₆ alkyl and C₃-C₆ cycloalkyl are each optionally substituted with one or more halogen;
R₄₇ is selected from H, C₁-C₆ alkyl, and C₃-C₆ cycloalkyl, wherein C₁-C₆ alkyl and C₃-C₆ cycloalkyl are each optionally substituted with one or more substituents selected from halogen and —OH; and
R₄₈ₐ and R_{48b} are each independently selected from H and C₁-C₆ alkyl.

In one embodiment, the compound of Formula (1-5007) is selected from Compounds 1-7 to 295-7 or Compounds 1a-7 to 79a-7.

| # | Structure |
|---|---|
| 1-7 | 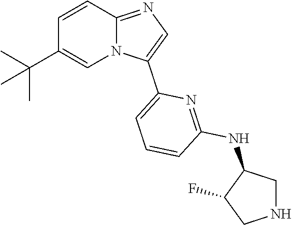 [SMILES: CC(C)(C)c1ccc2ncc(-c3cccc(N[C@H]4CNC[C@@H]4F)n3)n2c1] |
| 2-7 | 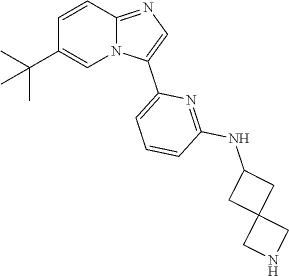 [SMILES: CC(C)(C)c1ccc2ncc(-c3cccc(NC4CC5(CNC5)C4)n3)n2c1] |
| 3-7 | 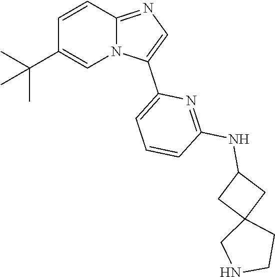 [SMILES: CC(C)(C)c1ccc2ncc(-c3cccc(NC4CC5(CCNC5)C4)n3)n2c1] |
| 4-7 | 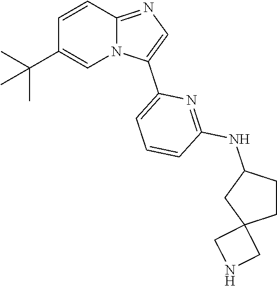 [SMILES: CC(C)(C)c1ccc2ncc(-c3cccc(NC4CCC5(CNC5)C4)n3)n2c1] |
| 5-7 | 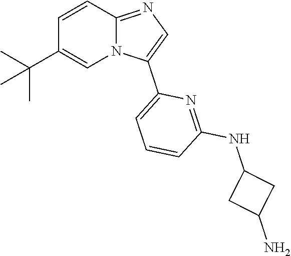 [SMILES: CC(C)(C)c1ccc2ncc(-c3cccc(NC4CC(N)C4)n3)n2c1] |
| 6-7 | 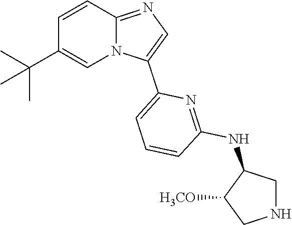 [SMILES: CO[C@H]1CNC[C@@H]1Nc1cccc(-c2cnc3ccc(C(C)(C)C)cn23)n1] |
| 7-7 | 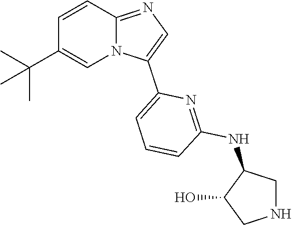 [SMILES: CC(C)(C)c1ccc2ncc(-c3cccc(N[C@H]4CNC[C@@H]4O)n3)n2c1] |
| 8-7 | 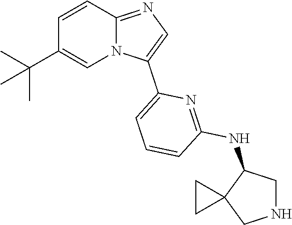 [SMILES: CC(C)(C)c1ccc2ncc(-c3cccc(N[C@H]4CNCC45CC5)n3)n2c1] |
| 9-7 | 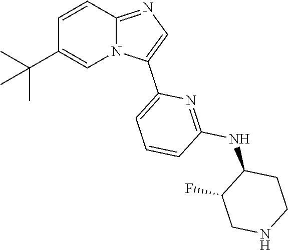 [SMILES: CC(C)(C)c1ccc2ncc(-c3cccc(N[C@H]4CCNC[C@@H]4F)n3)n2c1] |
| 10-7 | 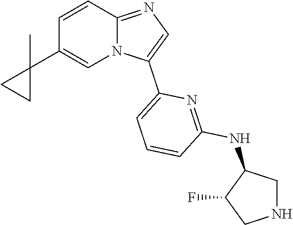 [SMILES: CC1(c2ccc3ncc(-c4cccc(N[C@H]5CNC[C@@H]5F)n4)n3c2)CC1] |
| 11-7 | 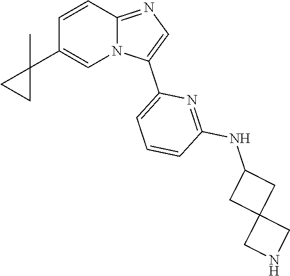 [SMILES: CC1(c2ccc3ncc(-c4cccc(NC5CC6(CNC6)C5)n4)n3c2)CC1] |
| 12-7 | 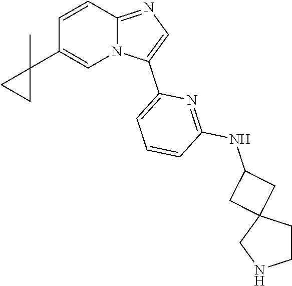 [SMILES: CC1(c2ccc3ncc(-c4cccc(NC5CC6(CCNC6)C5)n4)n3c2)CC1] |
| 13-7 | 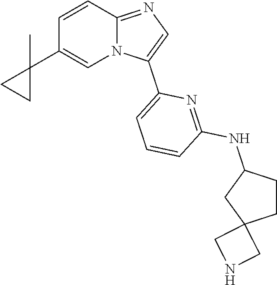 [SMILES: CC1(c2ccc3ncc(-c4cccc(NC5CCC6(CNC6)C5)n4)n3c2)CC1] |
| 14-7 | 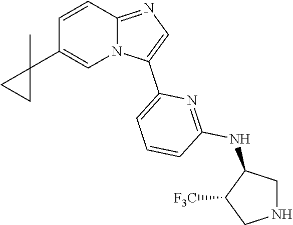 [SMILES: CC1(c2ccc3ncc(-c4cccc(N[C@H]5CNC[C@@H]5C(F)(F)F)n4)n3c2)CC1] |
| 15-7 | 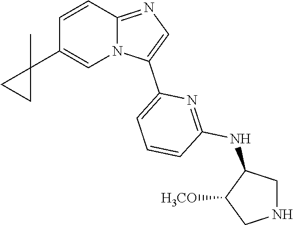 [SMILES: CO[C@H]1CNC[C@@H]1Nc1cccc(-c2cnc3ccc(C4(C)CC4)cn23)n1] |
| 16-7 | 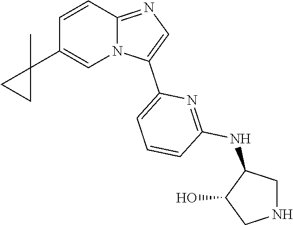 [SMILES: CC1(c2ccc3ncc(-c4cccc(N[C@H]5CNC[C@@H]5O)n4)n3c2)CC1] |
| 17-7 | 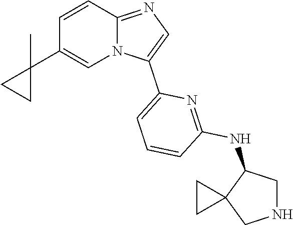 [SMILES: CC1(c2ccc3ncc(-c4cccc(N[C@H]5CNCC56CC6)n4)n3c2)CC1] |
| 18-7 | 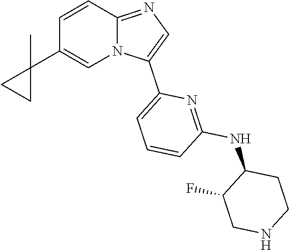 [SMILES: CC1(c2ccc3ncc(-c4cccc(N[C@H]5CCNC[C@@H]5F)n4)n3c2)CC1] |
| 19-7 | 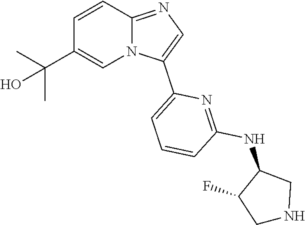 [SMILES: CC(C)(O)c1ccc2ncc(-c3cccc(N[C@H]4CNC[C@@H]4F)n3)n2c1] |
| 20-7 | 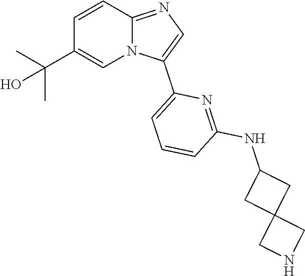 [SMILES: CC(C)(O)c1ccc2ncc(-c3cccc(NC4CC5(CNC5)C4)n3)n2c1] |
| 21-7 | 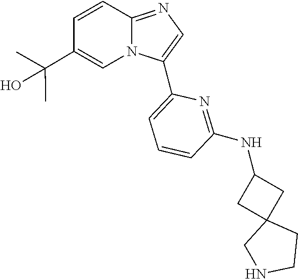 [SMILES: CC(C)(O)c1ccc2ncc(-c3cccc(NC4CC5(CCNC5)C4)n3)n2c1] |
| 22-7 | 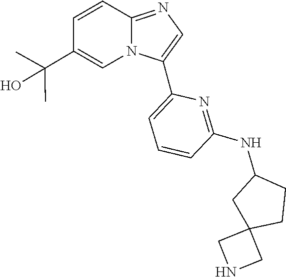 [SMILES: CC(C)(O)c1ccc2ncc(-c3cccc(NC4CCC5(CNC5)C4)n3)n2c1] |
| 23-7 | 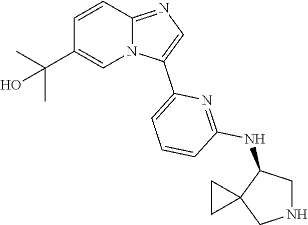 [SMILES: CC(C)(O)c1ccc2ncc(-c3cccc(N[C@H]4CNCC45CC5)n3)n2c1] |
| 24-7 | 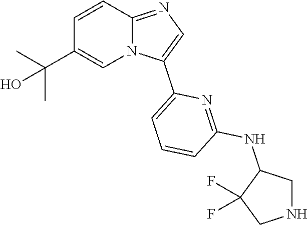 [SMILES: CC(C)(O)c1ccc2ncc(-c3cccc(NC4CNCC4(F)F)n3)n2c1] |
| 25-7 | 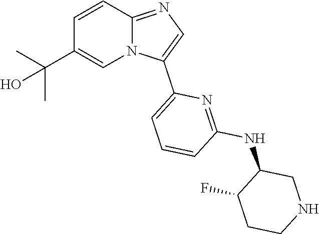 [SMILES: CC(C)(O)c1ccc2ncc(-c3cccc(N[C@H]4CNCC[C@@H]4F)n3)n2c1] |
| 26-7 | 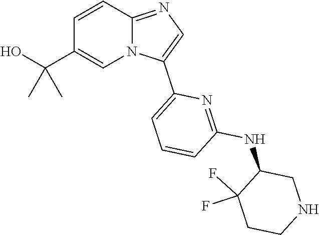 [SMILES: CC(C)(O)c1ccc2ncc(-c3cccc(N[C@H]4CNCCC4(F)F)n3)n2c1] |
| 27-7 | 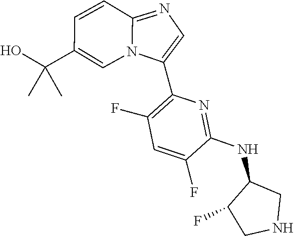 [SMILES: CC(C)(O)c1ccc2ncc(-c3nc(N[C@H]4CNC[C@@H]4F)c(F)cc3F)n2c1] |
| 28-7 | 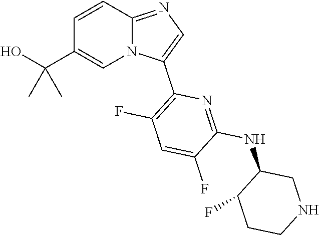 [SMILES: CC(C)(O)c1ccc2ncc(-c3nc(N[C@H]4CNCC[C@@H]4F)c(F)cc3F)n2c1] |
| 29-7 | 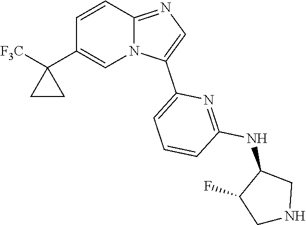 [SMILES: F[C@H]1CNC[C@@H]1Nc1cccc(-c2cnc3ccc(C4(C(F)(F)F)CC4)cn23)n1] |
| 30-7 | 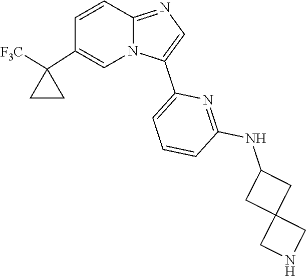 [SMILES: FC(F)(F)C1(c2ccc3ncc(-c4cccc(NC5CC6(CNC6)C5)n4)n3c2)CC1] |
| 31-7 | 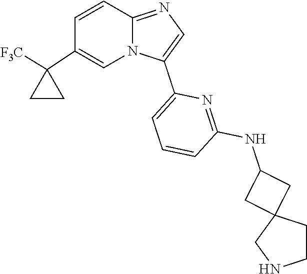 [SMILES: FC(F)(F)C1(c2ccc3ncc(-c4cccc(NC5CC6(CCNC6)C5)n4)n3c2)CC1] |
| 32-7 | 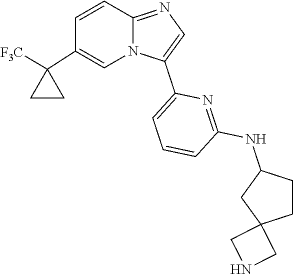 [SMILES: FC(F)(F)C1(c2ccc3ncc(-c4cccc(NC5CCC6(CNC6)C5)n4)n3c2)CC1] |
| 33-7 | 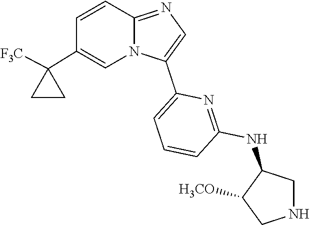 [SMILES: CO[C@H]1CNC[C@@H]1Nc1cccc(-c2cnc3ccc(C4(C(F)(F)F)CC4)cn23)n1] |
| 34-7 | 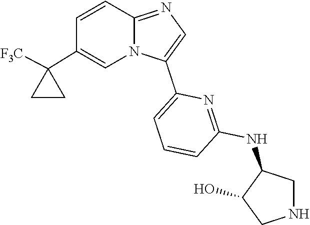 [SMILES: O[C@H]1CNC[C@@H]1Nc1cccc(-c2cnc3ccc(C4(C(F)(F)F)CC4)cn23)n1] |
| 35-7 | 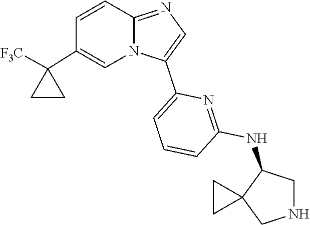 [SMILES: FC(F)(F)C1(c2ccc3ncc(-c4cccc(N[C@H]5CNCC56CC6)n4)n3c2)CC1] |
| 36-7 | 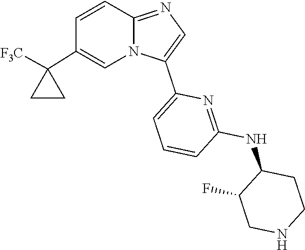 [SMILES: F[C@H]1CNCC[C@@H]1Nc1cccc(-c2cnc3ccc(C4(C(F)(F)F)CC4)cn23)n1] |
| 37-7 | 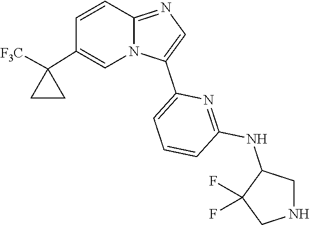 [SMILES: FC1(F)CNCC1Nc1cccc(-c2cnc3ccc(C4(C(F)(F)F)CC4)cn23)n1] |
| 38-7 | 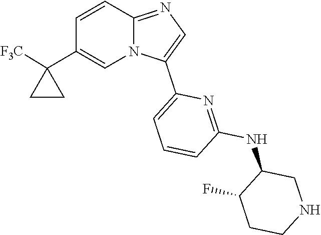 [SMILES: F[C@H]1CCNC[C@@H]1Nc1cccc(-c2cnc3ccc(C4(C(F)(F)F)CC4)cn23)n1] |
| 39-7 | 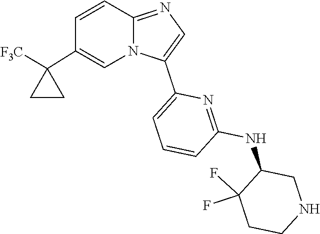 [SMILES: FC1(F)CCNC[C@@H]1Nc1cccc(-c2cnc3ccc(C4(C(F)(F)F)CC4)cn23)n1] |
| 41-7 | 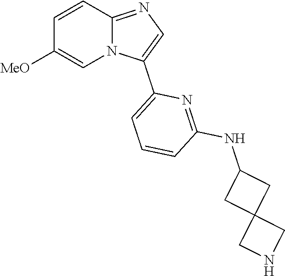 [SMILES: COc1ccc2ncc(-c3cccc(NC4CC5(CNC5)C4)n3)n2c1] |
| 42-7 | 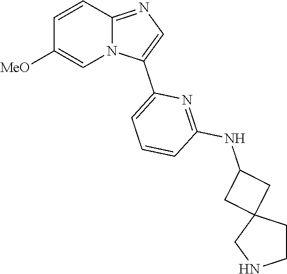 [SMILES: COc1ccc2ncc(-c3cccc(NC4CC5(CCNC5)C4)n3)n2c1] |
| 43-7 | 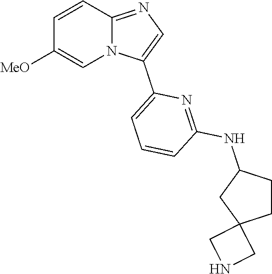 [SMILES: COc1ccc2ncc(-c3cccc(NC4CCC5(CNC5)C4)n3)n2c1] |
| 44-7 | 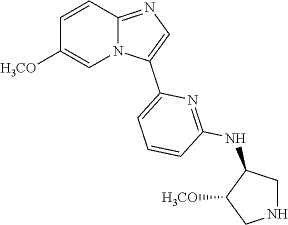 [SMILES: COc1ccc2ncc(-c3cccc(N[C@H]4CNC[C@@H]4OC)n3)n2c1] |
| 45-7 | 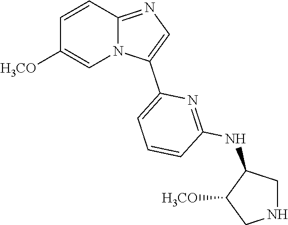 [SMILES: COc1ccc2ncc(-c3cccc(N[C@H]4CNC[C@@H]4OC)n3)n2c1] |
| 46-7 | 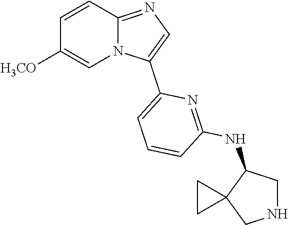 [SMILES: COc1ccc2ncc(-c3cccc(N[C@H]4CNCC45CC5)n3)n2c1] |
| 47-7 | 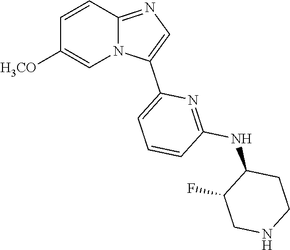 [SMILES: COc1ccc2ncc(-c3cccc(N[C@H]4CCNC[C@@H]4F)n3)n2c1] |
| 48-7 | 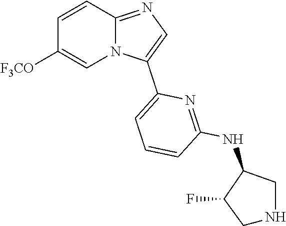 [SMILES: F[C@H]1CNC[C@@H]1Nc1cccc(-c2cnc3ccc(OC(F)(F)F)cn23)n1] |
| 49-7 | 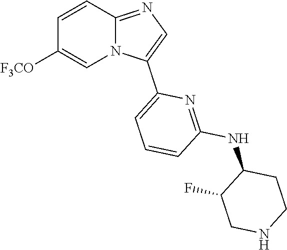 [SMILES: F[C@H]1CNCC[C@@H]1Nc1cccc(-c2cnc3ccc(OC(F)(F)F)cn23)n1] |
| 50-7 | 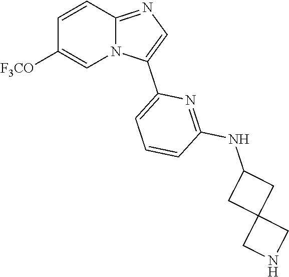 [SMILES: FC(F)(F)Oc1ccc2ncc(-c3cccc(NC4CC5(CNC5)C4)n3)n2c1] |
| 51-7 | 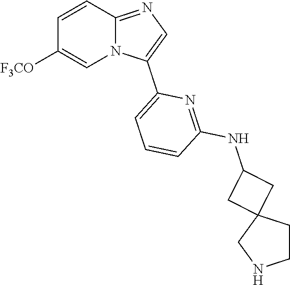 [SMILES: FC(F)(F)Oc1ccc2ncc(-c3cccc(NC4CC5(CCNC5)C4)n3)n2c1] |
| 52-7 | 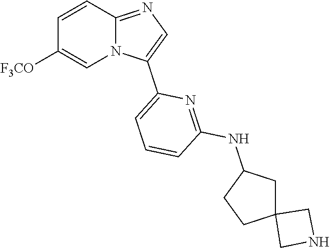 [SMILES: FC(F)(F)Oc1ccc2ncc(-c3cccc(NC4CCC5(CNC5)C4)n3)n2c1] |
| 53-7 | 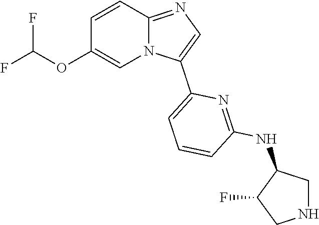 [SMILES: FC(F)Oc1ccc2ncc(-c3cccc(N[C@H]4CNC[C@@H]4F)n3)n2c1] |
| 54-7 | 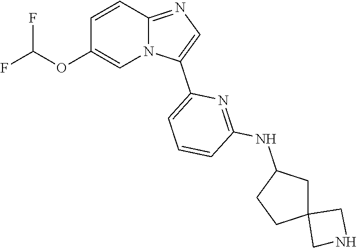 [SMILES: FC(F)Oc1ccc2ncc(-c3cccc(NC4CCC5(CNC5)C4)n3)n2c1] |
| 55-7 | 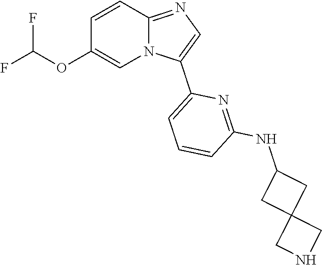 [SMILES: FC(F)Oc1ccc2ncc(-c3cccc(NC4CC5(CNC5)C4)n3)n2c1] |
| 56-7 | 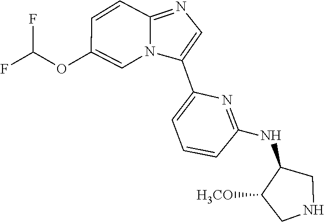 [SMILES: CO[C@H]1CNC[C@@H]1Nc1cccc(-c2cnc3ccc(OC(F)F)cn23)n1] |
| 57-7 | 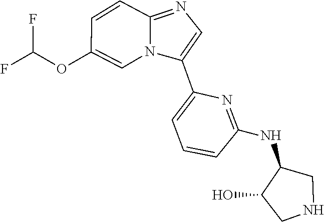 [SMILES: O[C@H]1CNC[C@@H]1Nc1cccc(-c2cnc3ccc(OC(F)F)cn23)n1] |
| 58-7 | 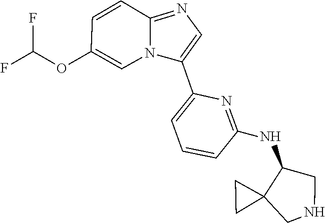 [SMILES: FC(F)Oc1ccc2ncc(-c3cccc(N[C@H]4CNCC45CC5)n3)n2c1] |
| 59-7 | 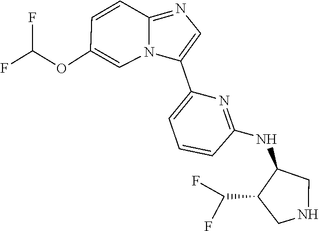 [SMILES: FC(F)Oc1ccc2ncc(-c3cccc(N[C@H]4CNC[C@@H]4C(F)F)n3)n2c1] |
| 60-7 | 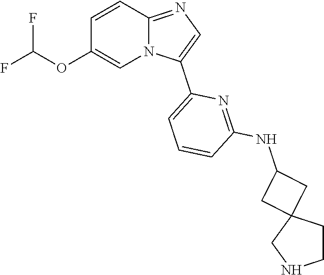 [SMILES: FC(F)Oc1ccc2ncc(-c3cccc(NC4CC5(CCNC5)C4)n3)n2c1] |
| 61-7 | 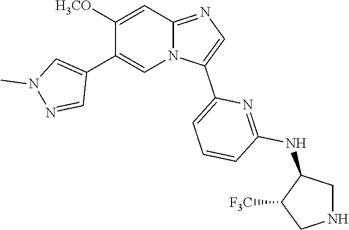 [SMILES: COc1cc2ncc(-c3cccc(N[C@H]4CNC[C@@H]4C(F)(F)F)n3)n2cc1-c1cnn(C)c1] |
| 62-7 | 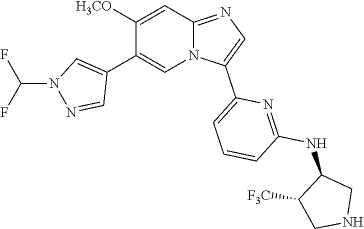 [SMILES: COc1cc2ncc(-c3cccc(N[C@H]4CNC[C@@H]4C(F)(F)F)n3)n2cc1-c1cnn(C(F)F)c1] |
| 63-7 | 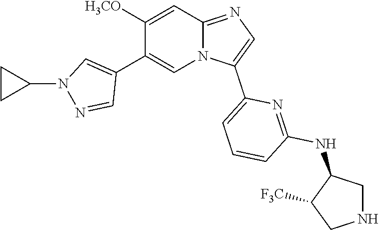 [SMILES: COc1cc2ncc(-c3cccc(N[C@H]4CNC[C@@H]4C(F)(F)F)n3)n2cc1-c1cnn(C2CC2)c1] |
| 64-7 | 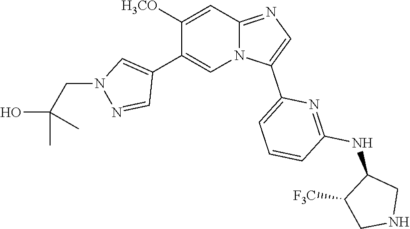 [SMILES: COc1cc2ncc(-c3cccc(N[C@H]4CNC[C@@H]4C(F)(F)F)n3)n2cc1-c1cnn(CC(C)(C)O)c1] |
| 65-7 | 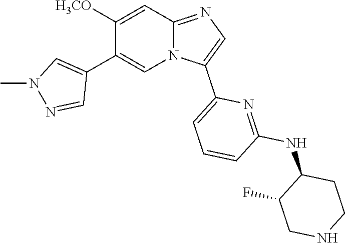 [SMILES: COc1cc2ncc(-c3cccc(N[C@H]4CCNC[C@@H]4F)n3)n2cc1-c1cnn(C)c1] |
| 66-7 | 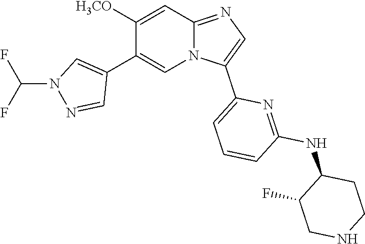 [SMILES: COc1cc2ncc(-c3cccc(N[C@H]4CCNC[C@@H]4F)n3)n2cc1-c1cnn(C(F)F)c1] |
| 67-7 | 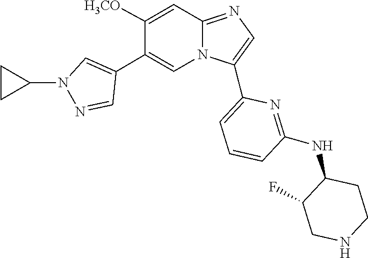 [SMILES: COc1cc2ncc(-c3cccc(N[C@H]4CCNC[C@@H]4F)n3)n2cc1-c1cnn(C2CC2)c1] |
| 68-7 | 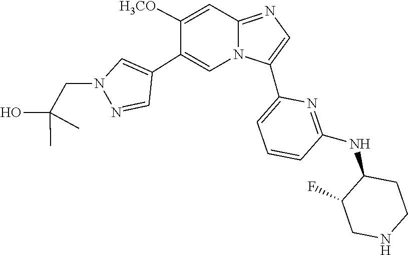 [SMILES: COc1cc2ncc(-c3cccc(N[C@H]4CCNC[C@@H]4F)n3)n2cc1-c1cnn(CC(C)(C)O)c1] |
| 69-7 | 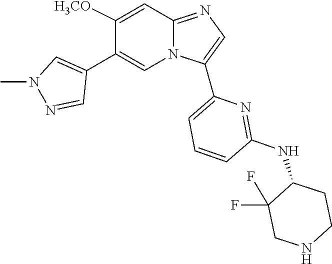 [SMILES: COc1cc2ncc(-c3cccc(N[C@@H]4CCNCC4(F)F)n3)n2cc1-c1cnn(C)c1] |
| 70-7 | 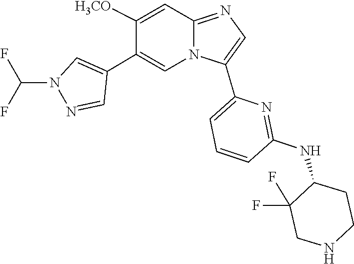 [SMILES: COc1cc2ncc(-c3cccc(N[C@@H]4CCNCC4(F)F)n3)n2cc1-c1cnn(C(F)F)c1] |
| 71-7 | 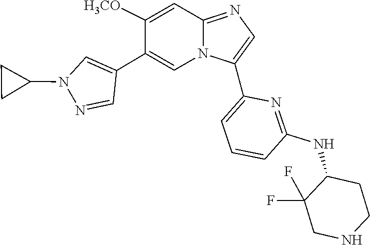 [SMILES: COc1cc2ncc(-c3cccc(N[C@@H]4CCNCC4(F)F)n3)n2cc1-c1cnn(C2CC2)c1] |
| 72-7 | 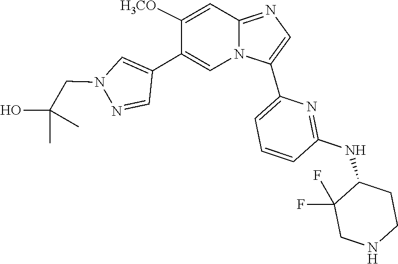 [SMILES: COc1cc2ncc(-c3cccc(N[C@@H]4CCNCC4(F)F)n3)n2cc1-c1cnn(CC(C)(C)O)c1] |
| 73-7 | 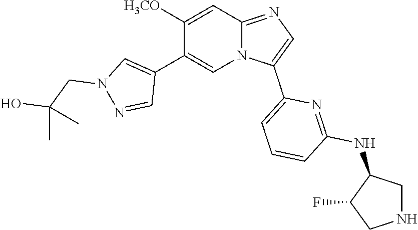 [SMILES: COc1cc2ncc(-c3cccc(N[C@H]4CNC[C@@H]4F)n3)n2cc1-c1cnn(CC(C)(C)O)c1] |
| 74-7 | 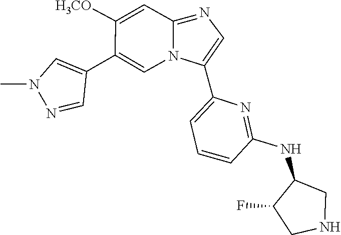 [SMILES: COc1cc2ncc(-c3cccc(N[C@H]4CNC[C@@H]4F)n3)n2cc1-c1cnn(C)c1] |
| 75-7 | 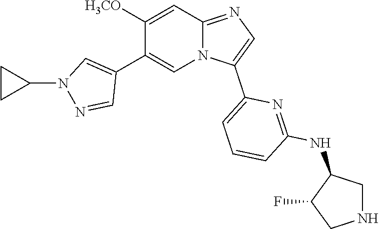 [SMILES: COc1cc2ncc(-c3cccc(N[C@H]4CNC[C@@H]4F)n3)n2cc1-c1cnn(C2CC2)c1] |
| 76-7 | 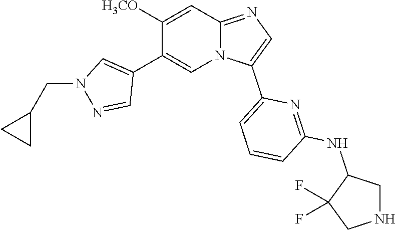 [SMILES: COc1cc2ncc(-c3cccc(NC4CNCC4(F)F)n3)n2cc1-c1cnn(CC2CC2)c1] |
| 77-7 | 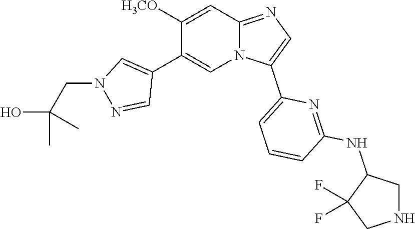 [SMILES: COc1cc2ncc(-c3cccc(NC4CNCC4(F)F)n3)n2cc1-c1cnn(CC(C)(C)O)c1] |
| 78-7 | 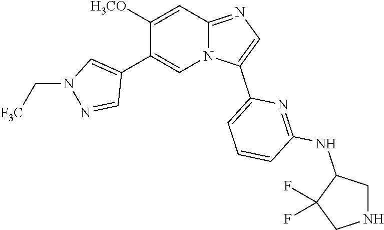 [SMILES: COc1cc2ncc(-c3cccc(NC4CNCC4(F)F)n3)n2cc1-c1cnn(CC(F)(F)F)c1] |
| 79-7 | 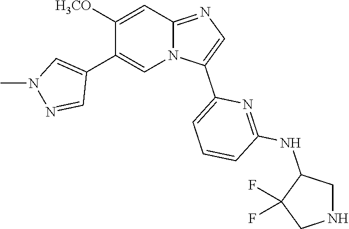 [SMILES: COc1cc2ncc(-c3cccc(NC4CNCC4(F)F)n3)n2cc1-c1cnn(C)c1] |
| 80-7 | 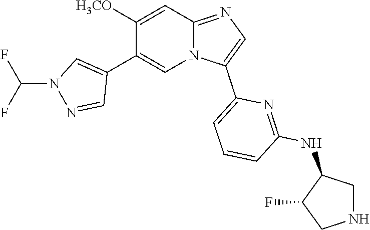 [SMILES: COc1cc2ncc(-c3cccc(N[C@H]4CNC[C@@H]4F)n3)n2cc1-c1cnn(C(F)F)c1] |
| 81-7 | 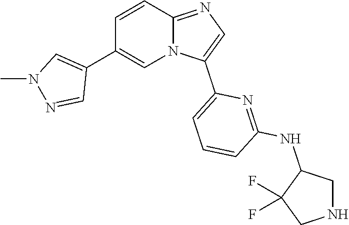 [SMILES: Cn1cc(-c2ccc3ncc(-c4cccc(NC5CNCC5(F)F)n4)n3c2)cn1] |
| 82-7 | 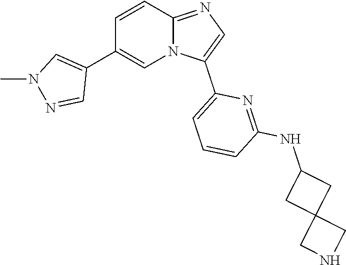 [SMILES: Cn1cc(-c2ccc3ncc(-c4cccc(NC5CC6(CNC6)C5)n4)n3c2)cn1] |
| 83-7 | 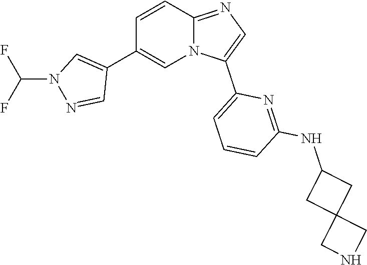 [SMILES: FC(F)n1cc(-c2ccc3ncc(-c4cccc(NC5CC6(CNC6)C5)n4)n3c2)cn1] |
| 84-7 | 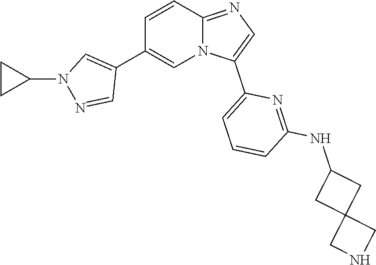 [SMILES: c1cc(NC2CC3(CNC3)C2)nc(-c2cnc3ccc(-c4cnn(C5CC5)c4)cn23)c1] |
| 85-7 | 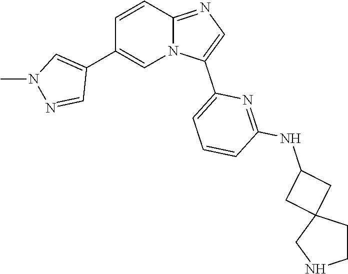 [SMILES: Cn1cc(-c2ccc3ncc(-c4cccc(NC5CC6(CCNC6)C5)n4)n3c2)cn1] |
| 86-7 | 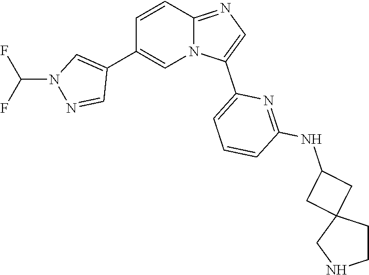 [SMILES: FC(F)n1cc(-c2ccc3ncc(-c4cccc(NC5CC6(CCNC6)C5)n4)n3c2)cn1] |
| 87-7 | 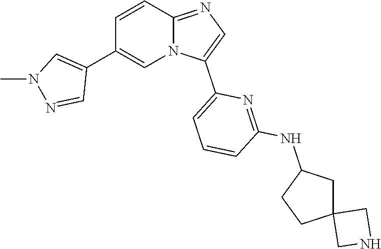 [SMILES: Cn1cc(-c2ccc3ncc(-c4cccc(NC5CCC6(CNC6)C5)n4)n3c2)cn1] |
| 88-7 | 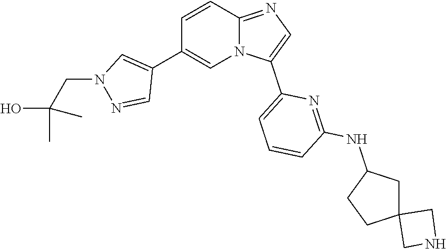 [SMILES: CC(C)(O)Cn1cc(-c2ccc3ncc(-c4cccc(NC5CCC6(CNC6)C5)n4)n3c2)cn1] |
| 89-7 | 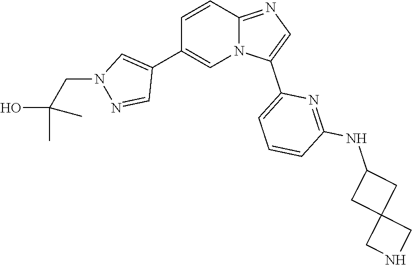 [SMILES: CC(C)(O)Cn1cc(-c2ccc3ncc(-c4cccc(NC5CC6(CNC6)C5)n4)n3c2)cn1] |
| 90-7 | 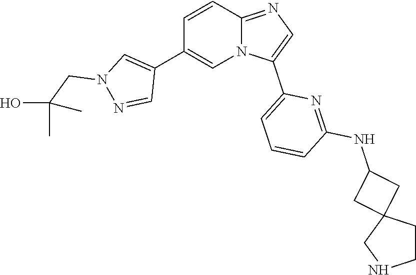 [SMILES: CC(C)(O)Cn1cc(-c2ccc3ncc(-c4cccc(NC5CC6(CCNC6)C5)n4)n3c2)cn1] |
| 91-7 | 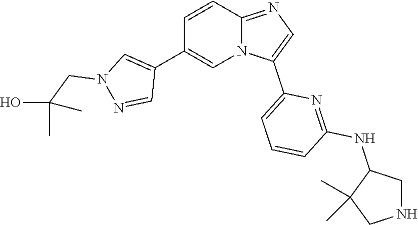 [SMILES: CC(C)(O)Cn1cc(-c2ccc3ncc(-c4cccc(NC5CNCC5(C)C)n4)n3c2)cn1] |
| 92-7 | 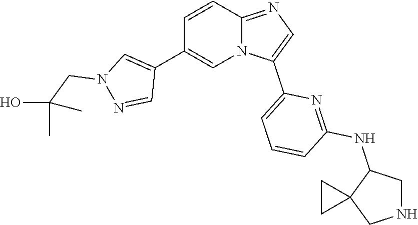 [SMILES: CC(C)(O)Cn1cc(-c2ccc3ncc(-c4cccc(NC5CNCC56CC6)n4)n3c2)cn1] |
| 93-7 | 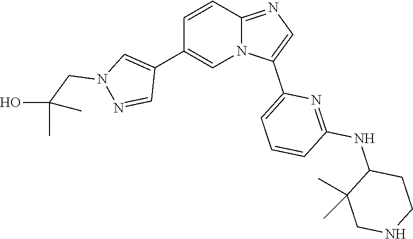 [SMILES: CC(C)(O)Cn1cc(-c2ccc3ncc(-c4cccc(NC5CCNCC5(C)C)n4)n3c2)cn1] |
| 94-7 | 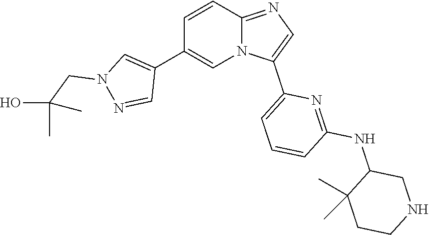 [SMILES: CC(C)(O)Cn1cc(-c2ccc3ncc(-c4cccc(NC5CNCCC5(C)C)n4)n3c2)cn1] |
| 95-7 | 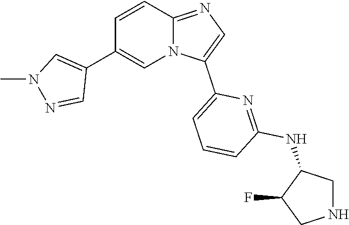 [SMILES: Cn1cc(-c2ccc3ncc(-c4cccc(N[C@@H]5CNC[C@H]5F)n4)n3c2)cn1] |
| 96-7 | 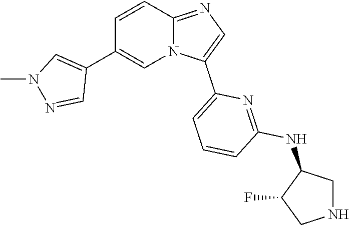 [SMILES: Cn1cc(-c2ccc3ncc(-c4cccc(N[C@H]5CNC[C@@H]5F)n4)n3c2)cn1] |
| 97-7 | 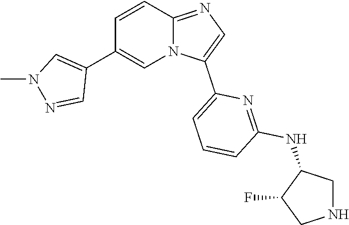 [SMILES: Cn1cc(-c2ccc3ncc(-c4cccc(N[C@@H]5CNC[C@@H]5F)n4)n3c2)cn1] |
| 98-7 | 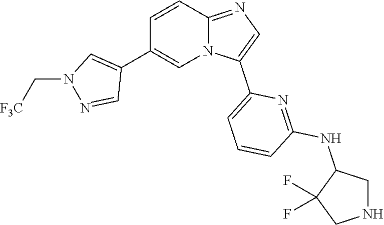 [SMILES: FC(F)(F)Cn1cc(-c2ccc3ncc(-c4cccc(NC5CNCC5(F)F)n4)n3c2)cn1] |
| 99-7 | 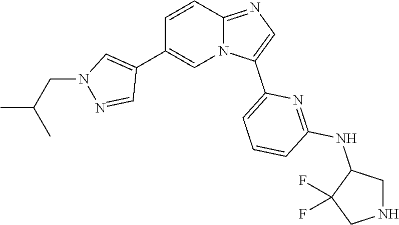 [SMILES: CC(C)Cn1cc(-c2ccc3ncc(-c4cccc(NC5CNCC5(F)F)n4)n3c2)cn1] |
| 100-7 | 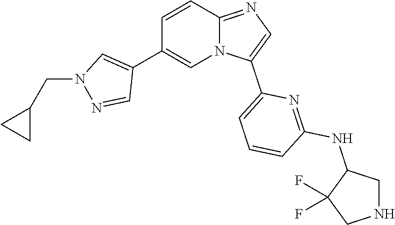 [SMILES: FC1(F)CNCC1Nc1cccc(-c2cnc3ccc(-c4cnn(CC5CC5)c4)cn23)n1] |
| 101-7 | 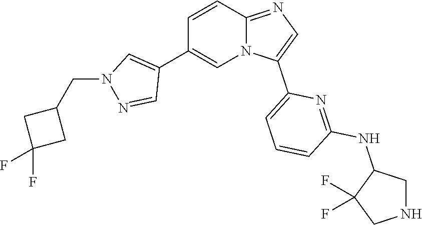 [SMILES: FC1(F)CC(Cn2cc(-c3ccc4ncc(-c5cccc(NC6CNCC6(F)F)n5)n4c3)cn2)C1] |
| 102-7 | 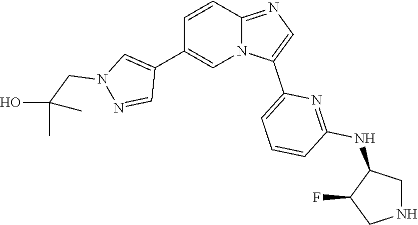 [SMILES: CC(C)(O)Cn1cc(-c2ccc3ncc(-c4cccc(N[C@H]5CNC[C@H]5F)n4)n3c2)cn1] |
| 103-7 | 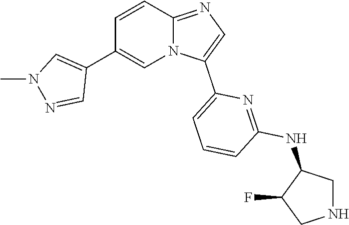 [SMILES: Cn1cc(-c2ccc3ncc(-c4cccc(N[C@H]5CNC[C@H]5F)n4)n3c2)cn1] |
| 104-7 | 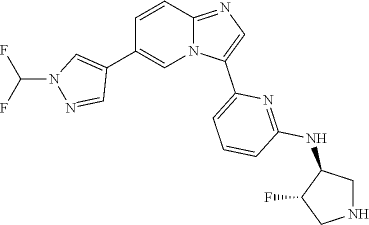 [SMILES: FC(F)n1cc(-c2ccc3ncc(-c4cccc(N[C@H]5CNC[C@@H]5F)n4)n3c2)cn1] |
| 105-7 | 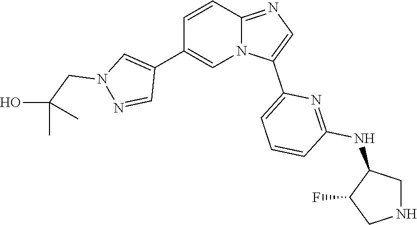 [SMILES: CC(C)(O)Cn1cc(-c2ccc3ncc(-c4cccc(N[C@H]5CNC[C@@H]5F)n4)n3c2)cn1] |
| 106-7 | 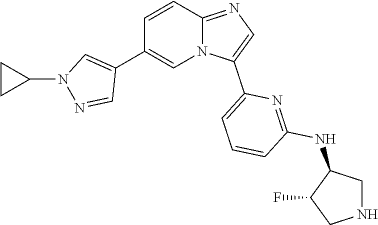 [SMILES: F[C@H]1CNC[C@@H]1Nc1cccc(-c2cnc3ccc(-c4cnn(C5CC5)c4)cn23)n1] |
| 107-7 | 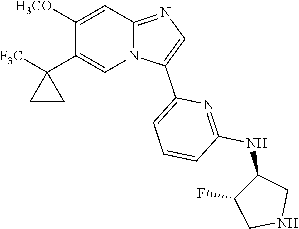 [SMILES: COc1cc2ncc(-c3cccc(N[C@H]4CNC[C@@H]4F)n3)n2cc1C1(C(F)(F)F)CC1] |
| 108-7 | 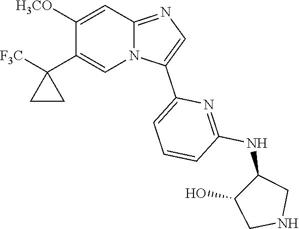 [SMILES: COc1cc2ncc(-c3cccc(N[C@H]4CNC[C@@H]4O)n3)n2cc1C1(C(F)(F)F)CC1] |
| 109-7 | 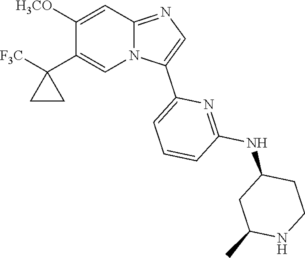 [SMILES: COc1cc2ncc(-c3cccc(N[C@H]4CCN[C@@H](C)C4)n3)n2cc1C1(C(F)(F)F)CC1] |
| 110-7 | 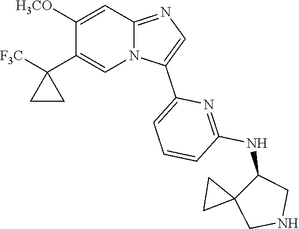 [SMILES: COc1cc2ncc(-c3cccc(N[C@H]4CNCC45CC5)n3)n2cc1C1(C(F)(F)F)CC1] |
| 111-7 | 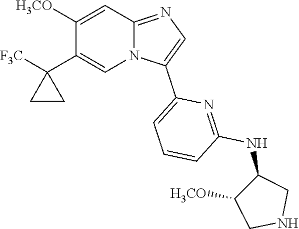 [SMILES: COc1cc2ncc(-c3cccc(N[C@H]4CNC[C@@H]4OC)n3)n2cc1C1(C(F)(F)F)CC1] |
| 112-7 | 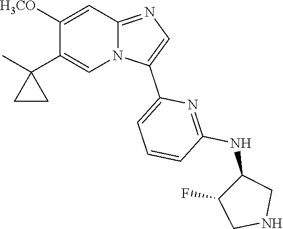 [SMILES: COc1cc2ncc(-c3cccc(N[C@H]4CNC[C@@H]4F)n3)n2cc1C1(C)CC1] |
| 113-7 | 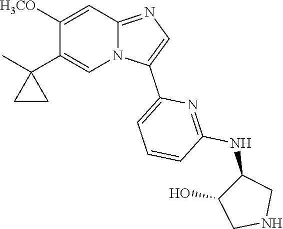 [SMILES: COc1cc2ncc(-c3cccc(N[C@H]4CNC[C@@H]4O)n3)n2cc1C1(C)CC1] |
| 114-7 | 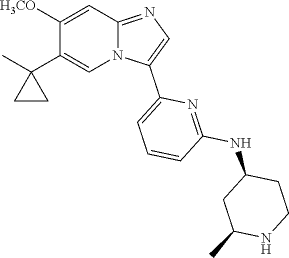 [SMILES: COc1cc2ncc(-c3cccc(N[C@H]4CCN[C@@H](C)C4)n3)n2cc1C1(C)CC1] |
| 115-7 | 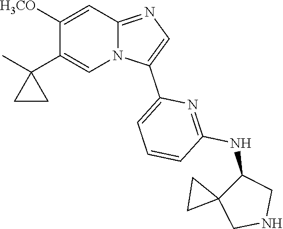 [SMILES: COc1cc2ncc(-c3cccc(N[C@H]4CNCC45CC5)n3)n2cc1C1(C)CC1] |
| 116-7 | 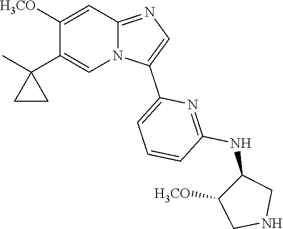 [SMILES: COc1cc2ncc(-c3cccc(N[C@H]4CNC[C@@H]4OC)n3)n2cc1C1(C)CC1] |
| 117-7 | 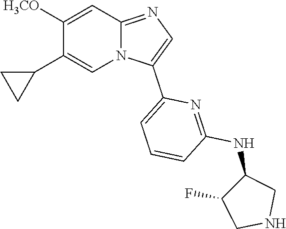 [SMILES: COc1cc2ncc(-c3cccc(N[C@H]4CNC[C@@H]4F)n3)n2cc1C1CC1] |
| 118-7 | 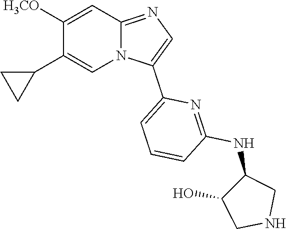 [SMILES: COc1cc2ncc(-c3cccc(N[C@H]4CNC[C@@H]4O)n3)n2cc1C1CC1] |
| 119-7 | 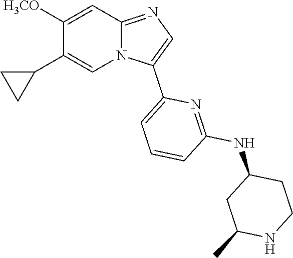 [SMILES: COc1cc2ncc(-c3cccc(N[C@H]4CCN[C@@H](C)C4)n3)n2cc1C1CC1] |
| 120-7 | 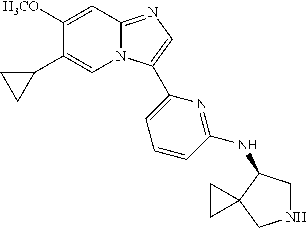 [SMILES: COc1cc2ncc(-c3cccc(N[C@H]4CNCC45CC5)n3)n2cc1C1CC1] |
| 121-7 | 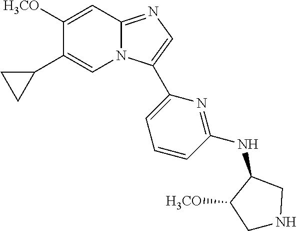 [SMILES: COc1cc2ncc(-c3cccc(N[C@H]4CNC[C@@H]4OC)n3)n2cc1C1CC1] |
| 122-7 | 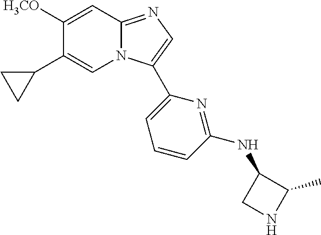 [SMILES: COc1cc2ncc(-c3cccc(N[C@@H]4CN[C@H]4C)n3)n2cc1C1CC1] |
| 123-7 | 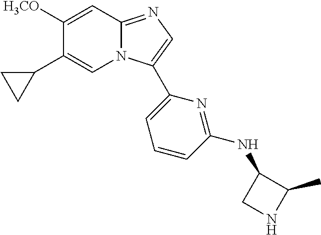 [SMILES: COc1cc2ncc(-c3cccc(N[C@@H]4CN[C@@H]4C)n3)n2cc1C1CC1] |
| 124-7 | 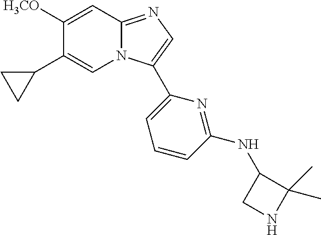 [SMILES: COc1cc2ncc(-c3cccc(NC4CNC4(C)C)n3)n2cc1C1CC1] |
| 125-7 | 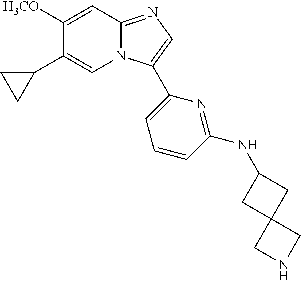 [SMILES: COc1cc2ncc(-c3cccc(NC4CC5(CNC5)C4)n3)n2cc1C1CC1] |
| 126-7 | 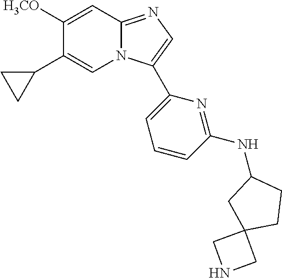 [SMILES: COc1cc2ncc(-c3cccc(NC4CCC5(CNC5)C4)n3)n2cc1C1CC1] |
| 127-7 | 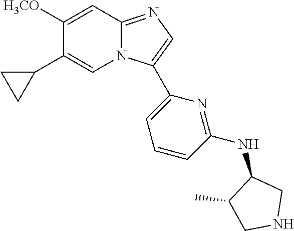 [SMILES: COc1cc2ncc(-c3cccc(N[C@H]4CNC[C@@H]4C)n3)n2cc1C1CC1] |
| 128-7 | 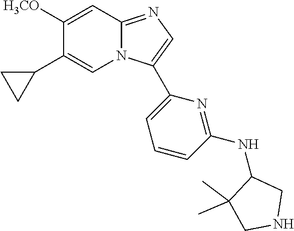 [SMILES: COc1cc2ncc(-c3cccc(NC4CNCC4(C)C)n3)n2cc1C1CC1] |
| 129-7 | 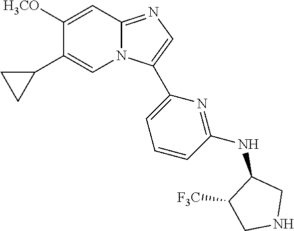 [SMILES: COc1cc2ncc(-c3cccc(N[C@H]4CNC[C@@H]4C(F)(F)F)n3)n2cc1C1CC1] |
| 130-7 | 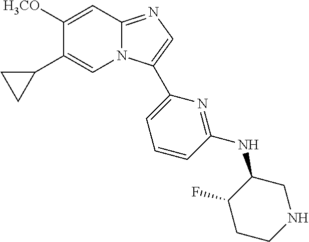 [SMILES: COc1cc2ncc(-c3cccc(N[C@H]4CNCC[C@@H]4F)n3)n2cc1C1CC1] |
| 131-7 | 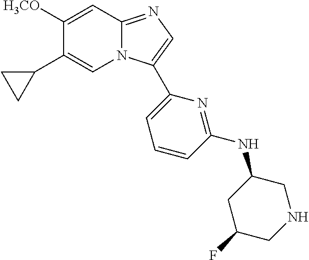 [SMILES: COc1cc2ncc(-c3cccc(N[C@H]4CNC[C@@H](F)C4)n3)n2cc1C1CC1] |
| 132-7 | 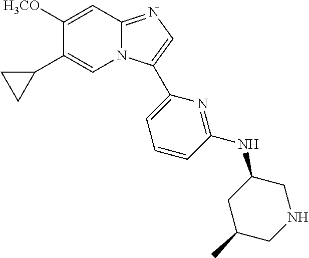 [SMILES: COc1cc2ncc(-c3cccc(N[C@H]4CNC[C@@H](C)C4)n3)n2cc1C1CC1] |
| 133-7 | 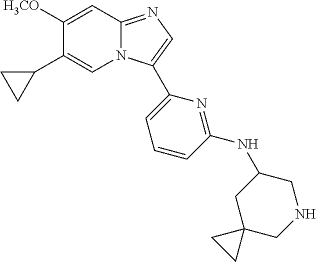 [SMILES: COc1cc2ncc(-c3cccc(NC4CNCC5(CC5)C4)n3)n2cc1C1CC1] |
| 134-7 | 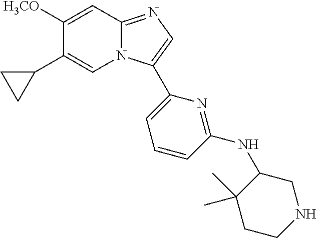 [SMILES: COc1cc2ncc(-c3cccc(NC4CNCCC4(C)C)n3)n2cc1C1CC1] |
| 135-7 | 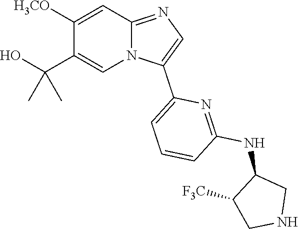 [SMILES: COc1cc2ncc(-c3cccc(N[C@H]4CNC[C@@H]4C(F)(F)F)n3)n2cc1C(C)(C)O] |
| 136-7 | 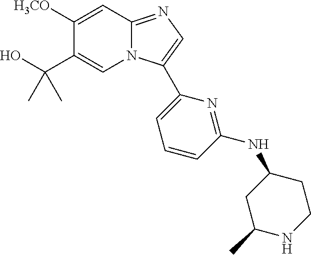 [SMILES: COc1cc2ncc(-c3cccc(N[C@H]4CCN[C@@H](C)C4)n3)n2cc1C(C)(C)O] |
| 137-7 | 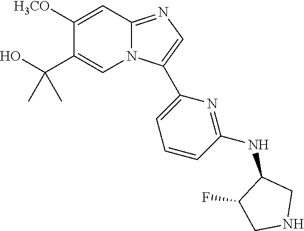 [SMILES: COc1cc2ncc(-c3cccc(N[C@H]4CNC[C@@H]4F)n3)n2cc1C(C)(C)O] |
| 138-7 | 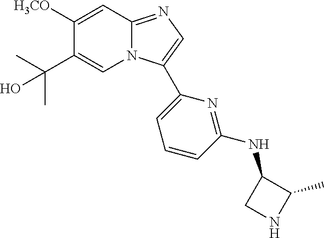 [SMILES: COc1cc2ncc(-c3cccc(N[C@@H]4CN[C@H]4C)n3)n2cc1C(C)(C)O] |
| 139-7 | 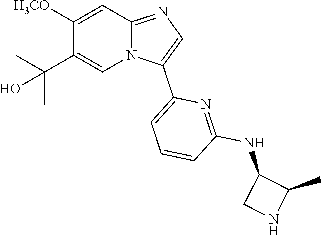 [SMILES: COc1cc2ncc(-c3cccc(N[C@@H]4CN[C@@H]4C)n3)n2cc1C(C)(C)O] |
| 140-7 | 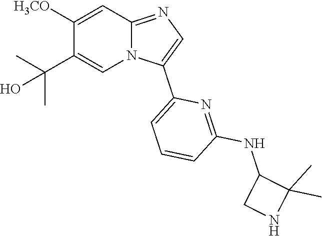 [SMILES: COc1cc2ncc(-c3cccc(NC4CNC4(C)C)n3)n2cc1C(C)(C)O] |
| 141-7 | 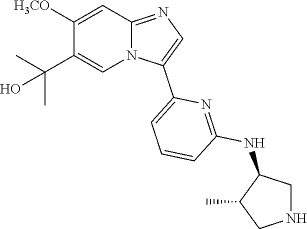 [SMILES: COc1cc2ncc(-c3cccc(N[C@H]4CNC[C@@H]4C)n3)n2cc1C(C)(C)O] |
| 142-7 | 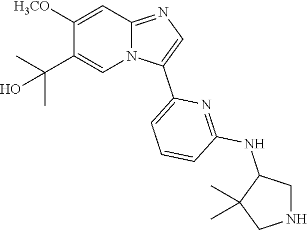 [SMILES: COc1cc2ncc(-c3cccc(NC4CNCC4(C)C)n3)n2cc1C(C)(C)O] |
| 143-7 | 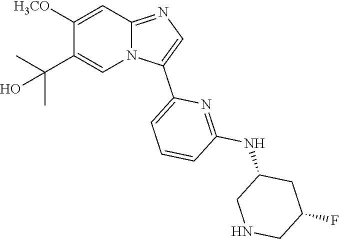 [SMILES: COc1cc2ncc(-c3cccc(N[C@H]4CNC[C@@H](F)C4)n3)n2cc1C(C)(C)O] |
| 144-7 | 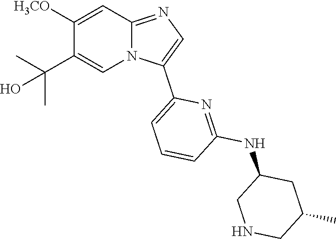 [SMILES: COc1cc2ncc(-c3cccc(N[C@@H]4CNC[C@@H](C)C4)n3)n2cc1C(C)(C)O] |
| 145-7 | 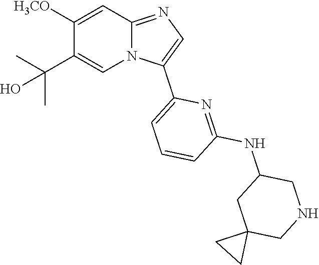 [SMILES: COc1cc2ncc(-c3cccc(NC4CNCC5(CC5)C4)n3)n2cc1C(C)(C)O] |
| 146-7 | 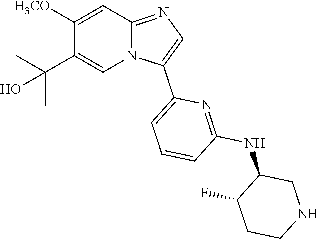 [SMILES: COc1cc2ncc(-c3cccc(N[C@H]4CNCC[C@@H]4F)n3)n2cc1C(C)(C)O] |
| 147-7 | 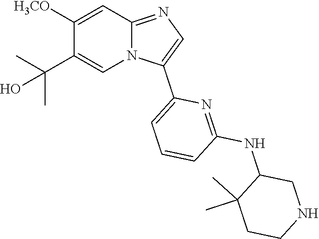 [SMILES: COc1cc2ncc(-c3cccc(NC4CNCCC4(C)C)n3)n2cc1C(C)(C)O] |
| 148-7 | 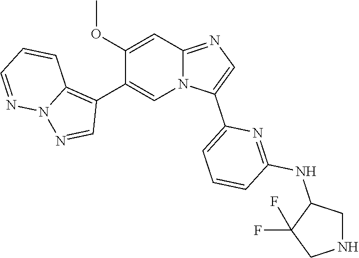 [SMILES: COc1cc2ncc(-c3cccc(NC4CNCC4(F)F)n3)n2cc1-c1cnn2ncccc12] |
| 149-7 | 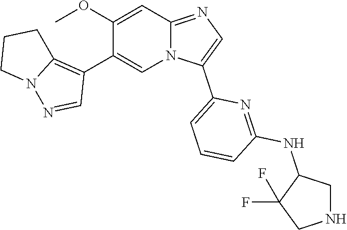 [SMILES: COc1cc2ncc(-c3cccc(NC4CNCC4(F)F)n3)n2cc1-c1cnn2c1CCC2] |
| 150-7 | 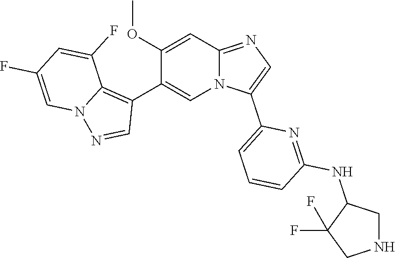 [SMILES: COc1cc2ncc(-c3cccc(NC4CNCC4(F)F)n3)n2cc1-c1cnn2cc(F)cc(F)c12] |
| 151-7 | 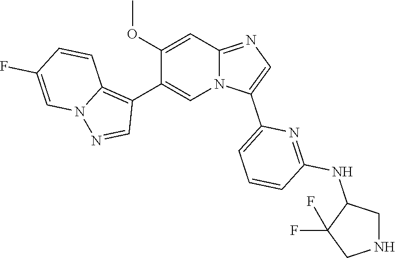 [SMILES: COc1cc2ncc(-c3cccc(NC4CNCC4(F)F)n3)n2cc1-c1cnn2cc(F)ccc12] |
| 152-7 | 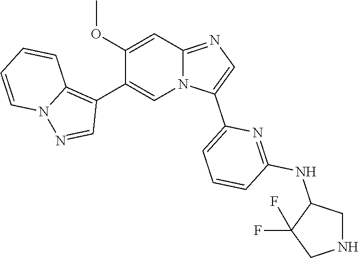 [SMILES: COc1cc2ncc(-c3cccc(NC4CNCC4(F)F)n3)n2cc1-c1cnn2ccccc12] |
| 153-7 | 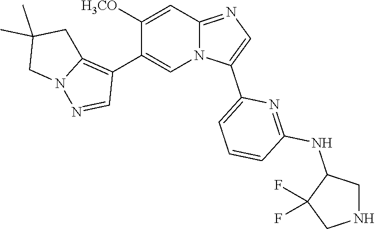 [SMILES: COc1cc2ncc(-c3cccc(NC4CNCC4(F)F)n3)n2cc1-c1cnn2c1CC(C)(C)C2] |
| 154-7 | 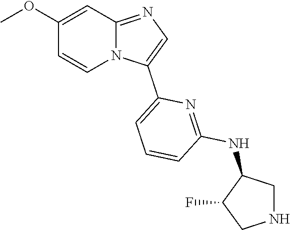 [SMILES: COc1ccn2c(-c3cccc(N[C@H]4CNC[C@@H]4F)n3)cnc2c1] |
| 155-7 | 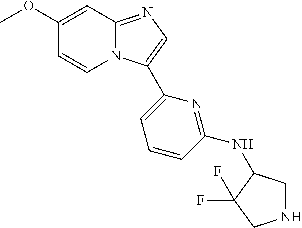 [SMILES: COc1ccn2c(-c3cccc(NC4CNCC4(F)F)n3)cnc2c1] |
| 156-7 | 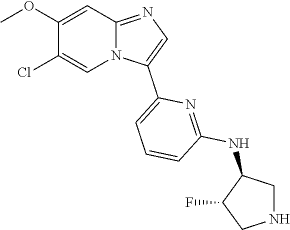 [SMILES: COc1cc2ncc(-c3cccc(N[C@H]4CNC[C@@H]4F)n3)n2cc1Cl] |
| 157-7 | 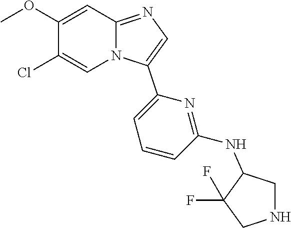 [SMILES: COc1cc2ncc(-c3cccc(NC4CNCC4(F)F)n3)n2cc1Cl] |
| 158-7 | 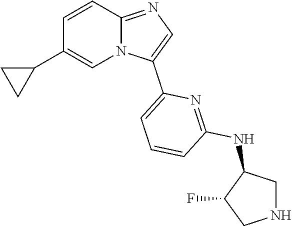 [SMILES: F[C@H]1CNC[C@@H]1Nc1cccc(-c2cnc3ccc(C4CC4)cn23)n1] |
| 159-7 | 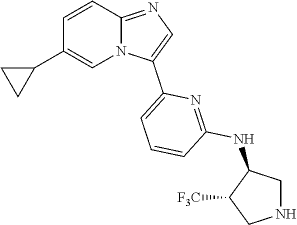 [SMILES: FC(F)(F)[C@H]1CNC[C@@H]1Nc1cccc(-c2cnc3ccc(C4CC4)cn23)n1] |
| 160-7 | 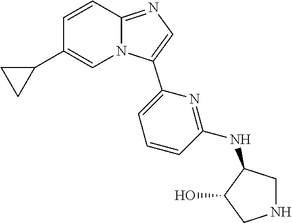 [SMILES: O[C@H]1CNC[C@@H]1Nc1cccc(-c2cnc3ccc(C4CC4)cn23)n1] |
| 161-7 | 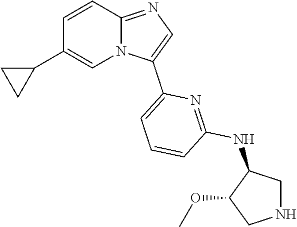 [SMILES: CO[C@H]1CNC[C@@H]1Nc1cccc(-c2cnc3ccc(C4CC4)cn23)n1] |
| 162-7 | 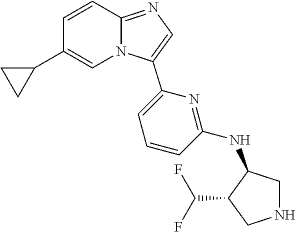 [SMILES: FC(F)[C@H]1CNC[C@@H]1Nc1cccc(-c2cnc3ccc(C4CC4)cn23)n1] |
| 163-7 | 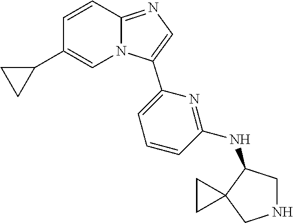 [SMILES: c1cc(N[C@H]2CNCC23CC3)nc(-c2cnc3ccc(C4CC4)cn23)c1] |
| 164-7 | 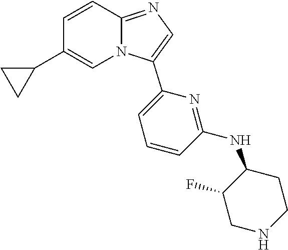 [SMILES: F[C@H]1CNCC[C@@H]1Nc1cccc(-c2cnc3ccc(C4CC4)cn23)n1] |
| 165-7 | 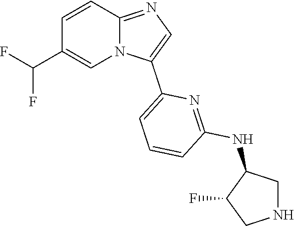 [SMILES: FC(F)c1ccc2ncc(-c3cccc(N[C@H]4CNC[C@@H]4F)n3)n2c1] |
| 166-7 | 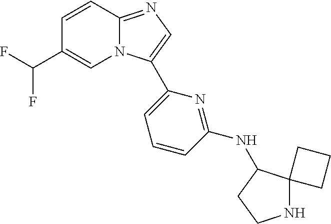 [SMILES: FC(F)c1ccc2ncc(-c3cccc(NC4CCNC45CCC5)n3)n2c1] |
| 167-7 | 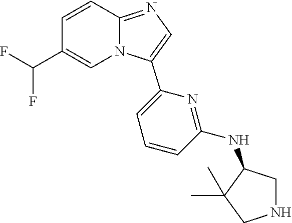 [SMILES: CC1(C)CNC[C@@H]1Nc1cccc(-c2cnc3ccc(C(F)F)cn23)n1] |
| 168-7 | 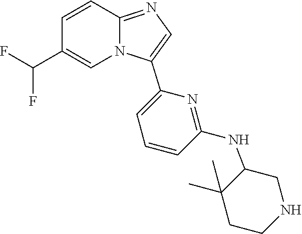 [SMILES: CC1(C)CCNCC1Nc1cccc(-c2cnc3ccc(C(F)F)cn23)n1] |
| 169-7 | 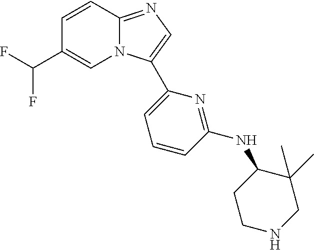 [SMILES: CC1(C)CNCC[C@H]1Nc1cccc(-c2cnc3ccc(C(F)F)cn23)n1] |
| 170-7 | 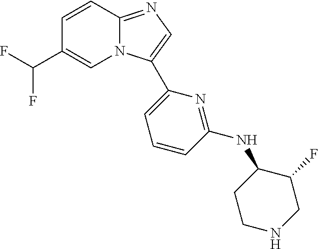 [SMILES: FC(F)c1ccc2ncc(-c3cccc(N[C@@H]4CCNC[C@H]4F)n3)n2c1] |
| 171-7 | 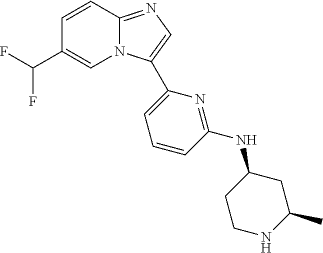 [SMILES: C[C@@H]1C[C@H](Nc2cccc(-c3cnc4ccc(C(F)F)cn34)n2)CCN1] |
| 172-7 | 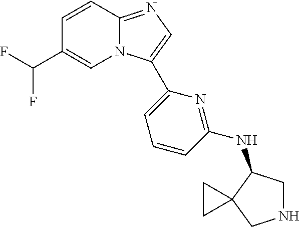 [SMILES: FC(F)c1ccc2ncc(-c3cccc(N[C@H]4CNCC45CC5)n3)n2c1] |
| 173-7 | 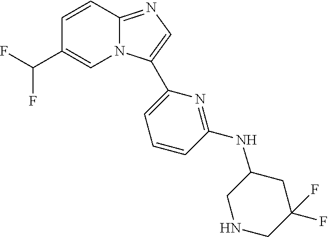 [SMILES: FC(F)c1ccc2ncc(-c3cccc(NC4CNCC(F)(F)C4)n3)n2c1] |
| 174-7 | 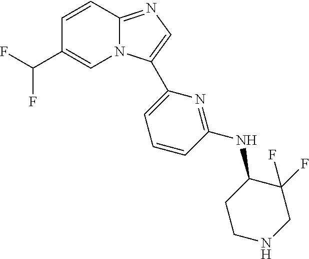 [SMILES: FC(F)c1ccc2ncc(-c3cccc(N[C@@H]4CCNCC4(F)F)n3)n2c1] |
| 175-7 | 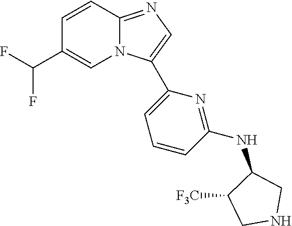 [SMILES: FC(F)c1ccc2ncc(-c3cccc(N[C@H]4CNC[C@@H]4C(F)(F)F)n3)n2c1] |
| 176-7 | 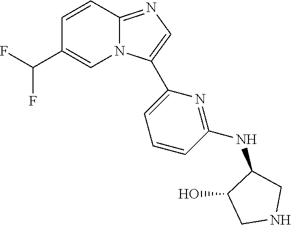 [SMILES: O[C@H]1CNC[C@@H]1Nc1cccc(-c2cnc3ccc(C(F)F)cn23)n1] |
| 177-7 | 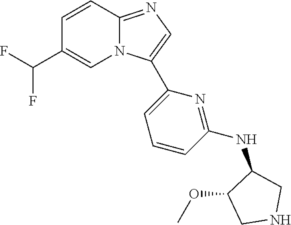 [SMILES: CO[C@H]1CNC[C@@H]1Nc1cccc(-c2cnc3ccc(C(F)F)cn23)n1] |
| 178-7 | 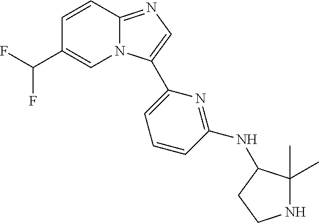 [SMILES: CC1(C)NCCC1Nc1cccc(-c2cnc3ccc(C(F)F)cn23)n1] |
| 179-7 | 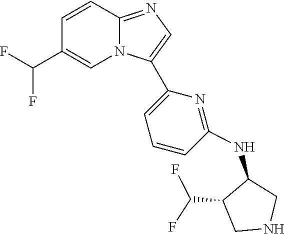 [SMILES: FC(F)c1ccc2ncc(-c3cccc(N[C@H]4CNC[C@@H]4C(F)F)n3)n2c1] |
| 180-7 | 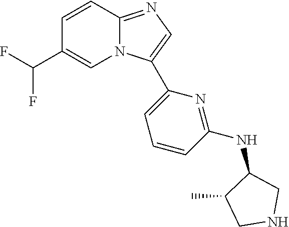 [SMILES: C[C@H]1CNC[C@@H]1Nc1cccc(-c2cnc3ccc(C(F)F)cn23)n1] |
| 181-7 | 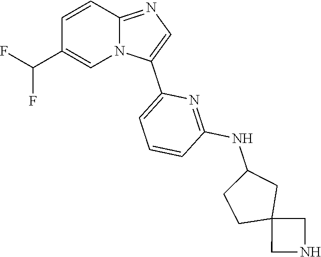 [SMILES: FC(F)c1ccc2ncc(-c3cccc(NC4CCC5(CNC5)C4)n3)n2c1] |
| 182-7 | 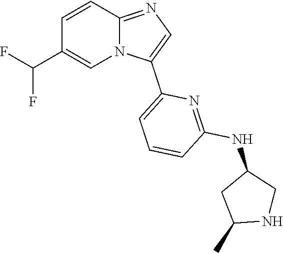 [SMILES: C[C@H]1C[C@@H](Nc2cccc(-c3cnc4ccc(C(F)F)cn34)n2)CN1] |
| 183-7 | 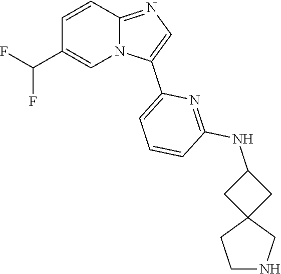 [SMILES: FC(F)c1ccc2ncc(-c3cccc(NC4CC5(CCNC5)C4)n3)n2c1] |
| 184-7 | 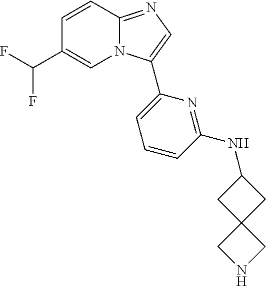 [SMILES: FC(F)c1ccc2ncc(-c3cccc(NC4CC5(CNC5)C4)n3)n2c1] |
| 185-7 | 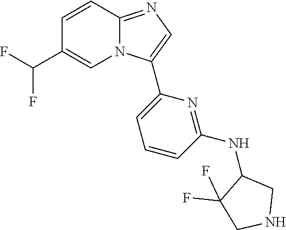 [SMILES: FC(F)c1ccc2ncc(-c3cccc(NC4CNCC4(F)F)n3)n2c1] |
| 186-7 | 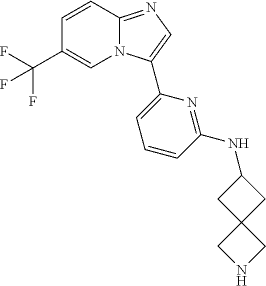 [SMILES: FC(F)(F)c1ccc2ncc(-c3cccc(NC4CC5(CNC5)C4)n3)n2c1] |
| 187-7 | 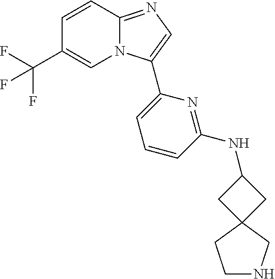 [SMILES: FC(F)(F)c1ccc2ncc(-c3cccc(NC4CC5(CCNC5)C4)n3)n2c1] |
| 188-7 | 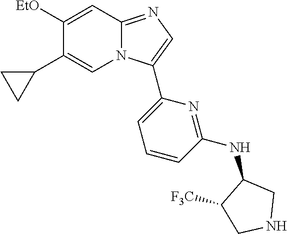 [SMILES: CCOc1cc2ncc(-c3cccc(N[C@H]4CNC[C@@H]4C(F)(F)F)n3)n2cc1C1CC1] |
| 189-7 | 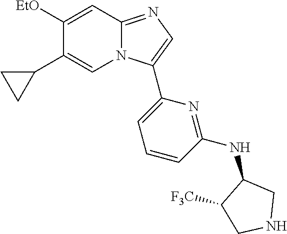 [SMILES: CCOc1cc2ncc(-c3cccc(N[C@H]4CNC[C@@H]4C(F)(F)F)n3)n2cc1C1CC1] |
| 190-7 | 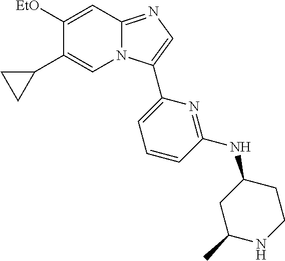 [SMILES: CCOc1cc2ncc(-c3cccc(N[C@H]4CCN[C@@H](C)C4)n3)n2cc1C1CC1] |
| 191-7 | 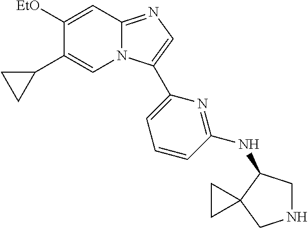 [SMILES: CCOc1cc2ncc(-c3cccc(N[C@H]4CNCC45CC5)n3)n2cc1C1CC1] |
| 192-7 | 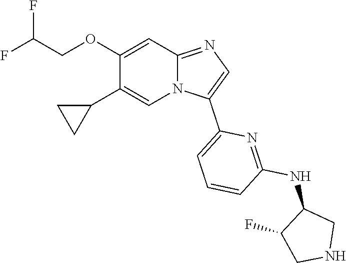 [SMILES: FC(F)COc1cc2ncc(-c3cccc(N[C@H]4CNC[C@@H]4F)n3)n2cc1C1CC1] |
| 193-7 | 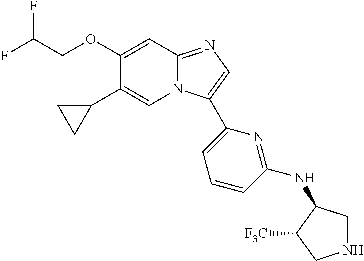 [SMILES: FC(F)COc1cc2ncc(-c3cccc(N[C@H]4CNC[C@@H]4C(F)(F)F)n3)n2cc1C1CC1] |
| 194-7 | 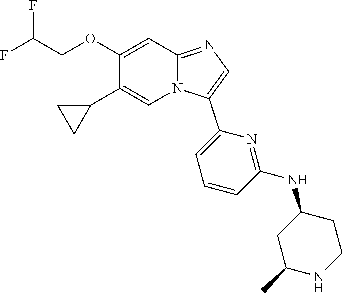 [SMILES: C[C@H]1C[C@@H](Nc2cccc(-c3cnc4cc(OCC(F)F)c(C5CC5)cn34)n2)CCN1] |
| 195-7 | 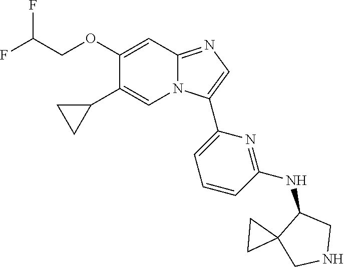 [SMILES: FC(F)COc1cc2ncc(-c3cccc(N[C@H]4CNCC45CC5)n3)n2cc1C1CC1] |
| 196-7 | 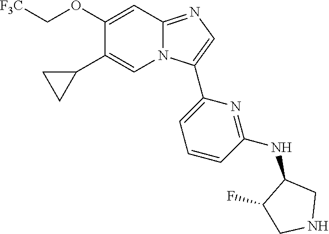 [SMILES: F[C@H]1CNC[C@@H]1Nc1cccc(-c2cnc3cc(OCC(F)(F)F)c(C4CC4)cn23)n1] |
| 197-7 | 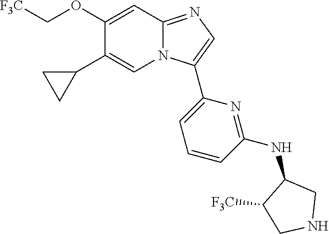 [SMILES: FC(F)(F)COc1cc2ncc(-c3cccc(N[C@H]4CNC[C@@H]4C(F)(F)F)n3)n2cc1C1CC1] |
| 198-7 | 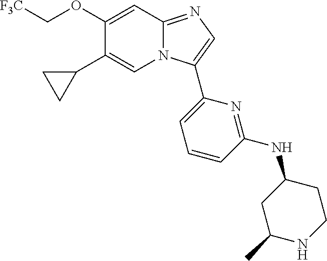 [SMILES: C[C@H]1C[C@@H](Nc2cccc(-c3cnc4cc(OCC(F)(F)F)c(C5CC5)cn34)n2)CCN1] |
| 199-7 | 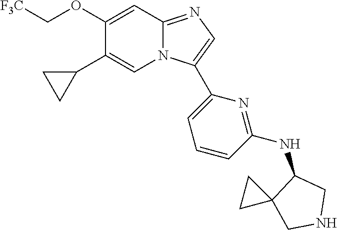 [SMILES: FC(F)(F)COc1cc2ncc(-c3cccc(N[C@H]4CNCC45CC5)n3)n2cc1C1CC1] |
| 200-7 | 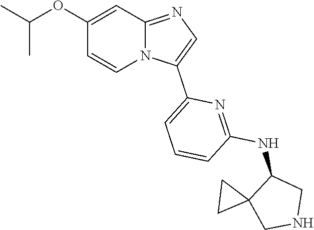 [SMILES: CC(C)Oc1ccn2c(-c3cccc(N[C@H]4CNCC45CC5)n3)cnc2c1] |
| 201-7 | 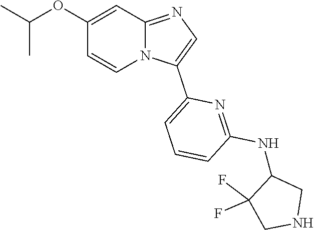 [SMILES: CC(C)Oc1ccn2c(-c3cccc(NC4CNCC4(F)F)n3)cnc2c1] |
| 202-7 | 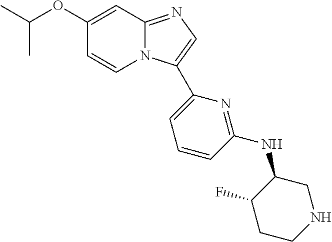 [SMILES: CC(C)Oc1ccn2c(-c3cccc(N[C@H]4CNCC[C@@H]4F)n3)cnc2c1] |
| 203-7 | 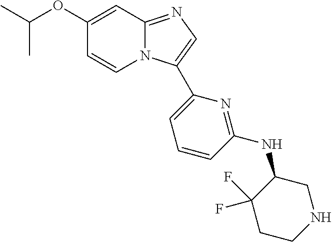 [SMILES: CC(C)Oc1ccn2c(-c3cccc(N[C@H]4CNCCC4(F)F)n3)cnc2c1] |
| 204-7 | 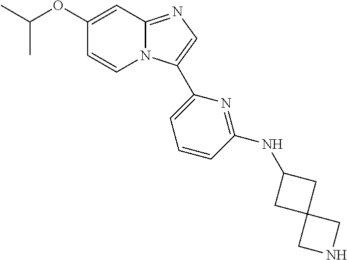 [SMILES: CC(C)Oc1ccn2c(-c3cccc(NC4CC5(CNC5)C4)n3)cnc2c1] |
| 205-7 | 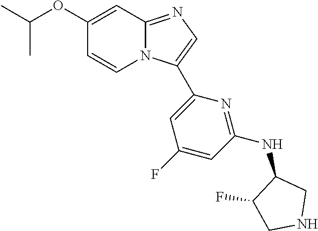 [SMILES: CC(C)Oc1ccn2c(-c3cc(F)cc(N[C@H]4CNC[C@@H]4F)n3)cnc2c1] |
| 206-7 | 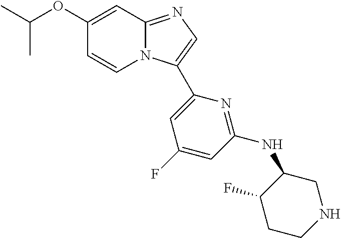 [SMILES: CC(C)Oc1ccn2c(-c3cc(F)cc(N[C@H]4CNCC[C@@H]4F)n3)cnc2c1] |
| 207-7 | 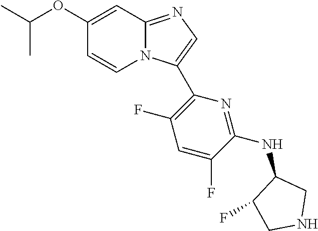 [SMILES: CC(C)Oc1ccn2c(-c3nc(N[C@H]4CNC[C@@H]4F)c(F)cc3F)cnc2c1] |
| 208-7 | 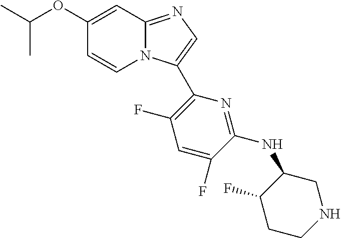 [SMILES: CC(C)Oc1ccn2c(-c3nc(N[C@H]4CNCC[C@@H]4F)c(F)cc3F)cnc2c1] |
| 209-7 | 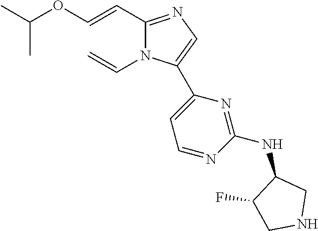 [SMILES: C=Cn1c(-c2ccnc(N[C@H]3CNC[C@@H]3F)n2)cnc1/C=C/OC(C)C] |
| 210-7 | 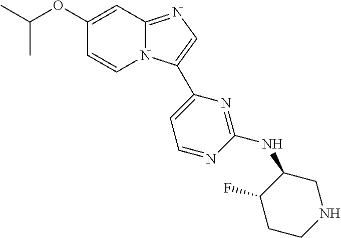 [SMILES: CC(C)Oc1ccn2c(-c3ccnc(N[C@H]4CNCC[C@@H]4F)n3)cnc2c1] |
| 211-7 | 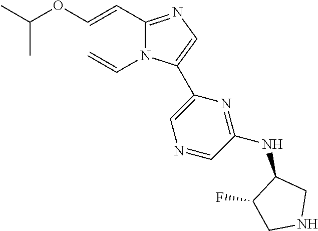 [SMILES: C=Cn1c(-c2cncc(N[C@H]3CNC[C@@H]3F)n2)cnc1/C=C/OC(C)C] |
| 212-7 | 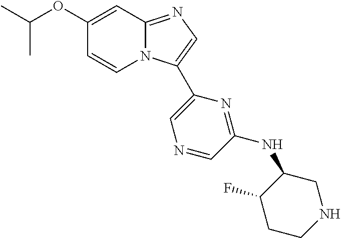 [SMILES: CC(C)Oc1ccn2c(-c3cncc(N[C@H]4CNCC[C@@H]4F)n3)cnc2c1] |
| 213-7 | 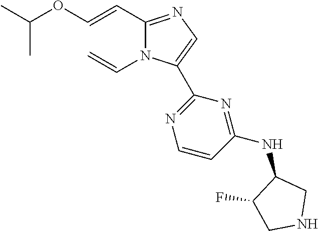 [SMILES: C=Cn1c(-c2nccc(N[C@H]3CNC[C@@H]3F)n2)cnc1/C=C/OC(C)C] |
| 214-7 | 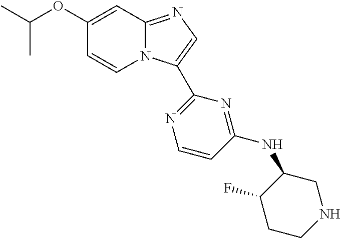 [SMILES: CC(C)Oc1ccn2c(-c3nccc(N[C@H]4CNCC[C@@H]4F)n3)cnc2c1] |
| 215-7 | 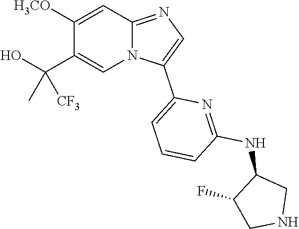 [SMILES: COc1cc2ncc(-c3cccc(N[C@H]4CNC[C@@H]4F)n3)n2cc1C(C)(O)C(F)(F)F] |
| 216-7 | 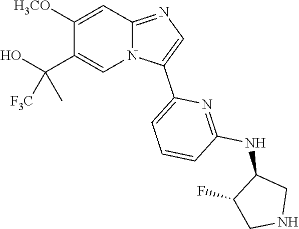 [SMILES: COc1cc2ncc(-c3cccc(N[C@H]4CNC[C@@H]4F)n3)n2cc1C(C)(O)C(F)(F)F] |
| 217-7 | 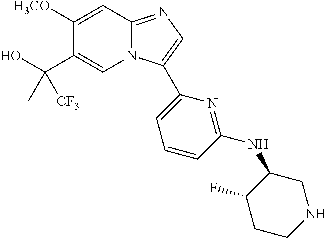 [SMILES: COc1cc2ncc(-c3cccc(N[C@H]4CNCC[C@@H]4F)n3)n2cc1C(C)(O)C(F)(F)F] |
| 218-7 | 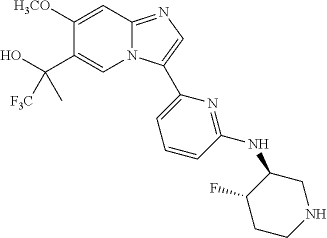 [SMILES: COc1cc2ncc(-c3cccc(N[C@H]4CNCC[C@@H]4F)n3)n2cc1C(C)(O)C(F)(F)F] |
| 219-7 | 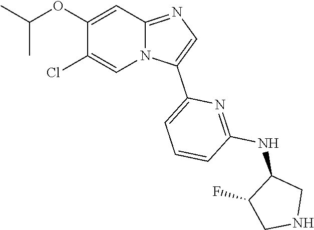 [SMILES: CC(C)Oc1cc2ncc(-c3cccc(N[C@H]4CNC[C@@H]4F)n3)n2cc1Cl] |
| 220-7 | 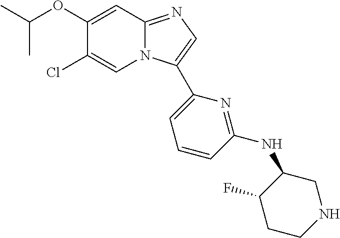 [SMILES: CC(C)Oc1cc2ncc(-c3cccc(N[C@H]4CNCC[C@@H]4F)n3)n2cc1Cl] |
| 221-7 | 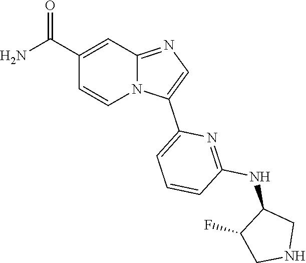 [SMILES: NC(=O)c1ccn2c(-c3cccc(N[C@H]4CNC[C@@H]4F)n3)cnc2c1] |
| 222-7 | 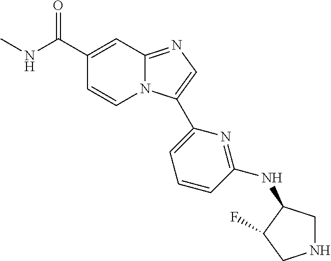 [SMILES: CNC(=O)c1ccn2c(-c3cccc(N[C@H]4CNC[C@@H]4F)n3)cnc2c1] |
| 223-7 | 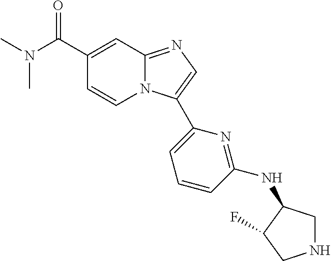 [SMILES: CN(C)C(=O)c1ccn2c(-c3cccc(N[C@H]4CNC[C@@H]4F)n3)cnc2c1] |
| 224-7 | 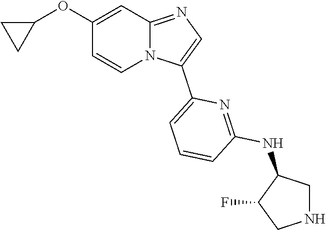 [SMILES: F[C@H]1CNC[C@@H]1Nc1cccc(-c2cnc3cc(OC4CC4)ccn23)n1] |
| 225-7 | 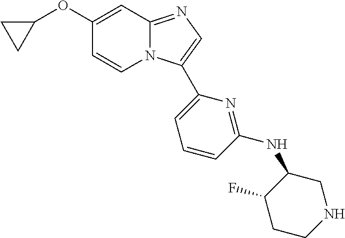 [SMILES: F[C@H]1CCNC[C@@H]1Nc1cccc(-c2cnc3cc(OC4CC4)ccn23)n1] |
| 226-7 | 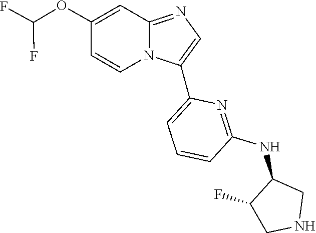 [SMILES: FC(F)Oc1ccn2c(-c3cccc(N[C@H]4CNC[C@@H]4F)n3)cnc2c1] |
| 227-7 | 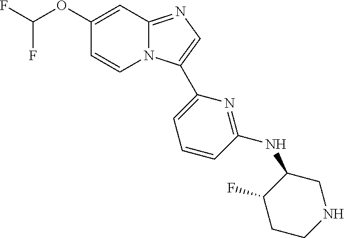 [SMILES: FC(F)Oc1ccn2c(-c3cccc(N[C@H]4CNCC[C@@H]4F)n3)cnc2c1] |
| 228-7 | 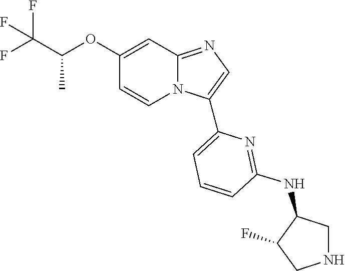 [SMILES: C[C@@H](Oc1ccn2c(-c3cccc(N[C@H]4CNC[C@@H]4F)n3)cnc2c1)C(F)(F)F] |
| 229-7 | 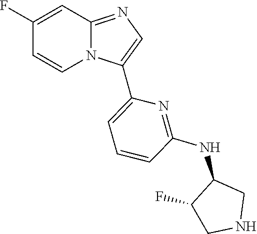 [SMILES: Fc1ccn2c(-c3cccc(N[C@H]4CNC[C@@H]4F)n3)cnc2c1] |
| 230-7 | 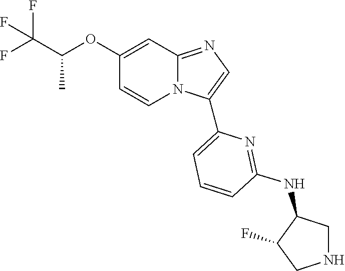 [SMILES: C[C@@H](Oc1ccn2c(-c3cccc(N[C@H]4CNC[C@@H]4F)n3)cnc2c1)C(F)(F)F] |
| 231-7 | 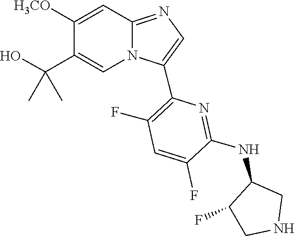 [SMILES: COc1cc2ncc(-c3nc(N[C@H]4CNC[C@@H]4F)c(F)cc3F)n2cc1C(C)(C)O] |
| 232-7 | 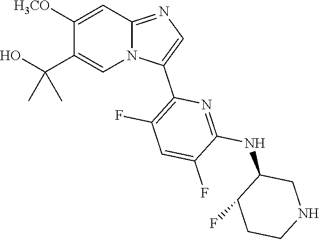 [SMILES: COc1cc2ncc(-c3nc(N[C@H]4CNCC[C@@H]4F)c(F)cc3F)n2cc1C(C)(C)O] |
| 233-7 | 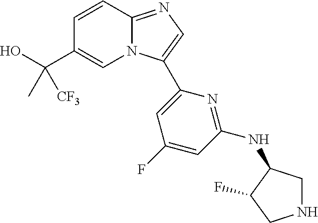 [SMILES: CC(O)(c1ccc2ncc(-c3cc(F)cc(N[C@H]4CNC[C@@H]4F)n3)n2c1)C(F)(F)F] |
| 234-7 | 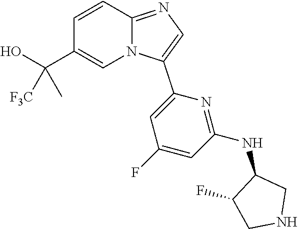 [SMILES: CC(O)(c1ccc2ncc(-c3cc(F)cc(N[C@H]4CNC[C@@H]4F)n3)n2c1)C(F)(F)F] |
| 235-7 | 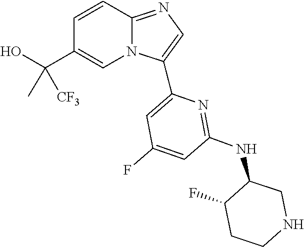 [SMILES: CC(O)(c1ccc2ncc(-c3cc(F)cc(N[C@H]4CNCC[C@@H]4F)n3)n2c1)C(F)(F)F] |
| 236-7 | 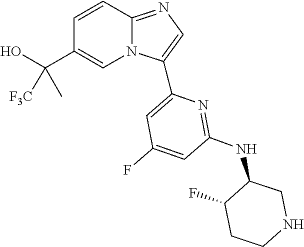 [SMILES: CC(O)(c1ccc2ncc(-c3cc(F)cc(N[C@H]4CNCC[C@@H]4F)n3)n2c1)C(F)(F)F] |
| 237-7 | 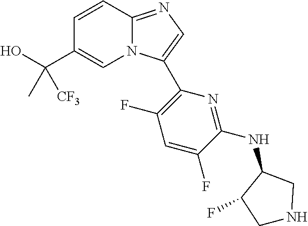 [SMILES: CC(O)(c1ccc2ncc(-c3nc(N[C@H]4CNC[C@@H]4F)c(F)cc3F)n2c1)C(F)(F)F] |
| 238-7 | 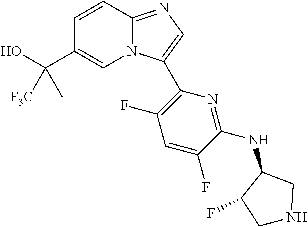 [SMILES: CC(O)(c1ccc2ncc(-c3nc(N[C@H]4CNC[C@@H]4F)c(F)cc3F)n2c1)C(F)(F)F] |
| 239-7 | 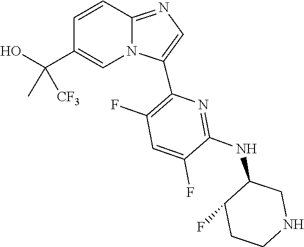 [SMILES: CC(O)(c1ccc2ncc(-c3nc(N[C@H]4CNCC[C@@H]4F)c(F)cc3F)n2c1)C(F)(F)F] |
| 240-7 | 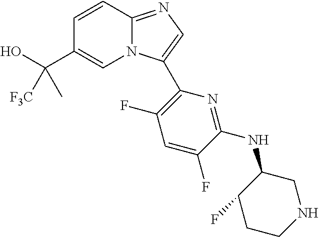 [SMILES: CC(O)(c1ccc2ncc(-c3nc(N[C@H]4CNCC[C@@H]4F)c(F)cc3F)n2c1)C(F)(F)F] |
| 241-7 | 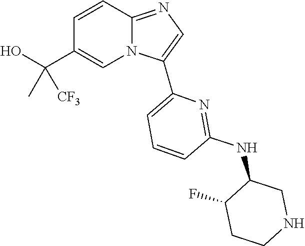 [SMILES: CC(O)(c1ccc2ncc(-c3cccc(N[C@H]4CNCC[C@@H]4F)n3)n2c1)C(F)(F)F] |
| 242-7 | 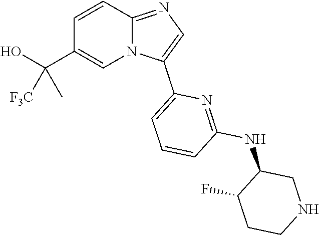 [SMILES: CC(O)(c1ccc2ncc(-c3cccc(N[C@H]4CNCC[C@@H]4F)n3)n2c1)C(F)(F)F] |
| 243-7 | 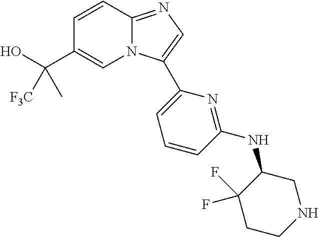 [SMILES: CC(O)(c1ccc2ncc(-c3cccc(N[C@H]4CNCCC4(F)F)n3)n2c1)C(F)(F)F] |
| 244-7 | 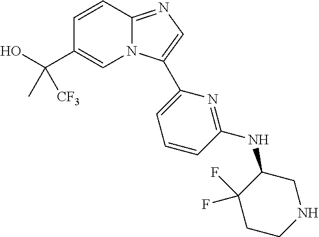 [SMILES: CC(O)(c1ccc2ncc(-c3cccc(N[C@H]4CNCCC4(F)F)n3)n2c1)C(F)(F)F] |
| 245-7 | 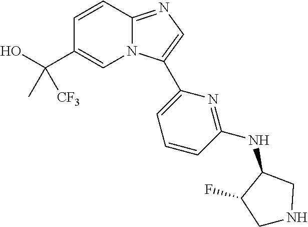 [SMILES: CC(O)(c1ccc2ncc(-c3cccc(N[C@H]4CNC[C@@H]4F)n3)n2c1)C(F)(F)F] |
| 246-7 | 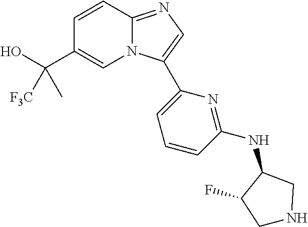 [SMILES: CC(O)(c1ccc2ncc(-c3cccc(N[C@H]4CNC[C@@H]4F)n3)n2c1)C(F)(F)F] |
| 247-7 | 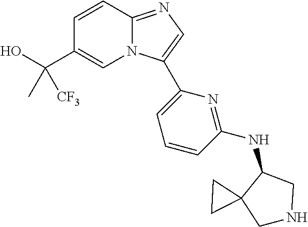 [SMILES: CC(O)(c1ccc2ncc(-c3cccc(N[C@H]4CNCC45CC5)n3)n2c1)C(F)(F)F] |
| 248-7 | 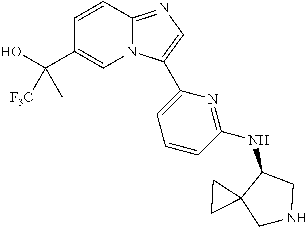 [SMILES: CC(O)(c1ccc2ncc(-c3cccc(N[C@H]4CNCC45CC5)n3)n2c1)C(F)(F)F] |
| 249-7 | 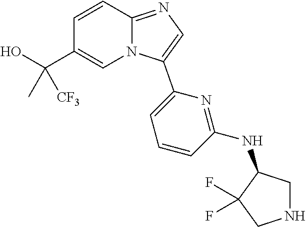 [SMILES: CC(O)(c1ccc2ncc(-c3cccc(N[C@H]4CNCC4(F)F)n3)n2c1)C(F)(F)F] |
| 250-7 | 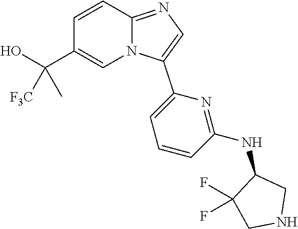 [SMILES: CC(O)(c1ccc2ncc(-c3cccc(N[C@H]4CNCC4(F)F)n3)n2c1)C(F)(F)F] |
| 251-7 | 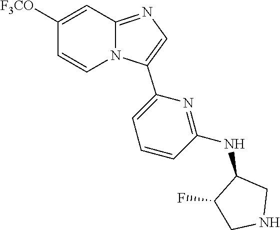 [SMILES: F[C@H]1CNC[C@@H]1Nc1cccc(-c2cnc3cc(OC(F)(F)F)ccn23)n1] |
| 252-7 | 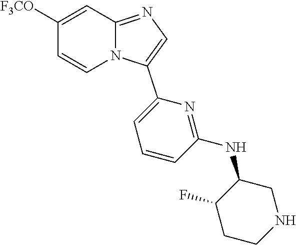 [SMILES: F[C@H]1CCNC[C@@H]1Nc1cccc(-c2cnc3cc(OC(F)(F)F)ccn23)n1] |
| 253-7 | 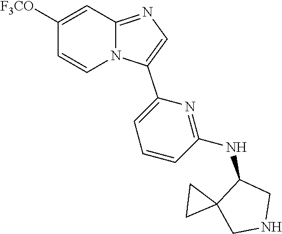 [SMILES: FC(F)(F)Oc1ccn2c(-c3cccc(N[C@H]4CNCC45CC5)n3)cnc2c1] |
| 254-7 | 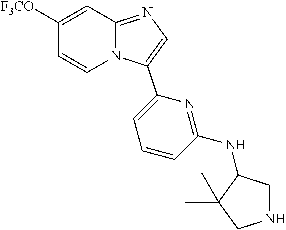 [SMILES: CC1(C)CNCC1Nc1cccc(-c2cnc3cc(OC(F)(F)F)ccn23)n1] |
| 255-7 | 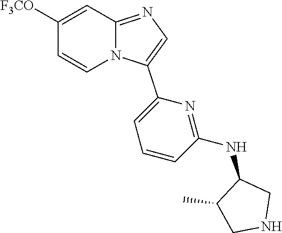 [SMILES: C[C@H]1CNC[C@@H]1Nc1cccc(-c2cnc3cc(OC(F)(F)F)ccn23)n1] |
| 256-7 | 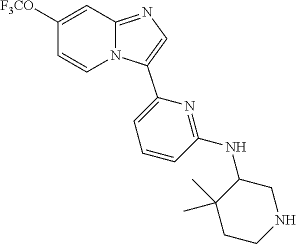 [SMILES: CC1(C)CCNCC1Nc1cccc(-c2cnc3cc(OC(F)(F)F)ccn23)n1] |
| 257-7 | 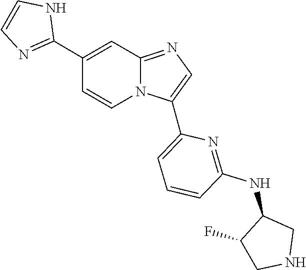 [SMILES: F[C@H]1CNC[C@@H]1Nc1cccc(-c2cnc3cc(-c4ncc[nH]4)ccn23)n1] |
| 259-7 | 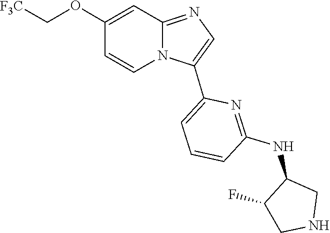 [SMILES: F[C@H]1CNC[C@@H]1Nc1cccc(-c2cnc3cc(OCC(F)(F)F)ccn23)n1] |
| 260-7 | 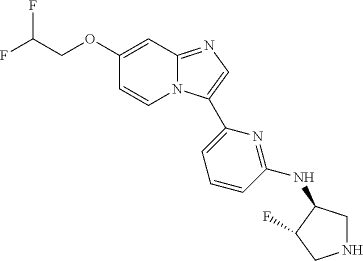 [SMILES: FC(F)COc1ccn2c(-c3cccc(N[C@H]4CNC[C@@H]4F)n3)cnc2c1] |
| 261-7 | 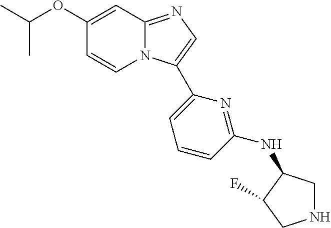 [SMILES: CC(C)Oc1ccn2c(-c3cccc(N[C@H]4CNC[C@@H]4F)n3)cnc2c1] |
| 262-7 | 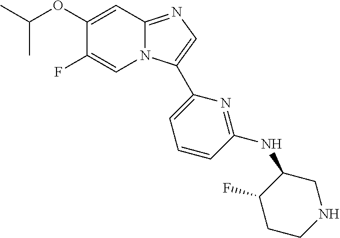 [SMILES: CC(C)Oc1cc2ncc(-c3cccc(N[C@H]4CNCC[C@@H]4F)n3)n2cc1F] |
| 263-7 | 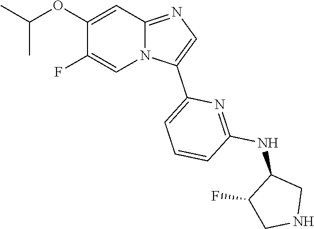 [SMILES: CC(C)Oc1cc2ncc(-c3cccc(N[C@H]4CNC[C@@H]4F)n3)n2cc1F] |
| 264-7 | 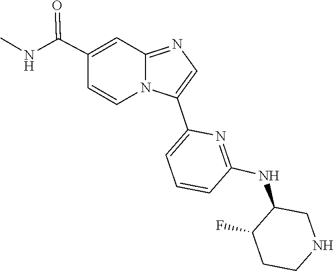 [SMILES: CNC(=O)c1ccn2c(-c3cccc(N[C@H]4CNCC[C@@H]4F)n3)cnc2c1] |
| 265-7 | 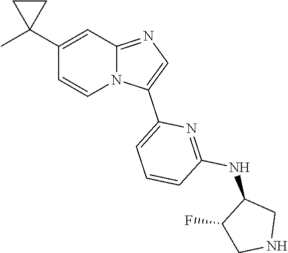 [SMILES: CC1(c2ccn3c(-c4cccc(N[C@H]5CNC[C@@H]5F)n4)cnc3c2)CC1] |
| 266-7 | 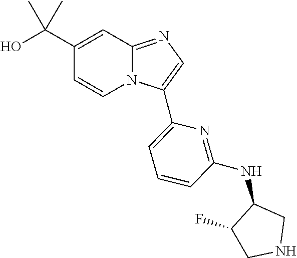 [SMILES: CC(C)(O)c1ccn2c(-c3cccc(N[C@H]4CNC[C@@H]4F)n3)cnc2c1] |
| 267-7 | 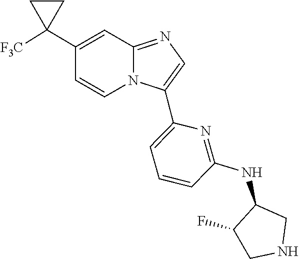 [SMILES: F[C@H]1CNC[C@@H]1Nc1cccc(-c2cnc3cc(C4(C(F)(F)F)CC4)ccn23)n1] |
| 268-7 | 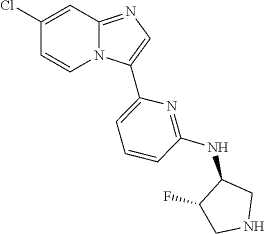 [SMILES: F[C@H]1CNC[C@@H]1Nc1cccc(-c2cnc3cc(Cl)ccn23)n1] |
| 269-7 | 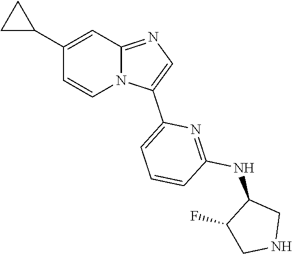 [SMILES: F[C@H]1CNC[C@@H]1Nc1cccc(-c2cnc3cc(C4CC4)ccn23)n1] |
| 270-7 | 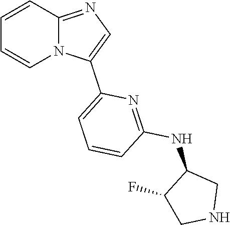 [SMILES: F[C@H]1CNC[C@@H]1Nc1cccc(-c2cnc3ccccn23)n1] |
| 271-7 | 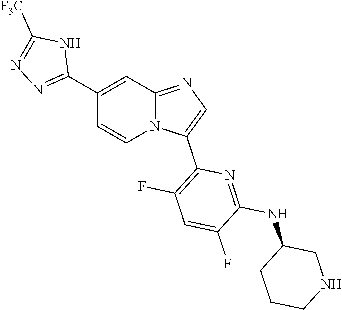 [SMILES: Fc1cc(F)c(-c2cnc3cc(-c4nnc(C(F)(F)F)[nH]4)ccn23)nc1N[C@@H]1CCCNC1] |
| 272-7 | 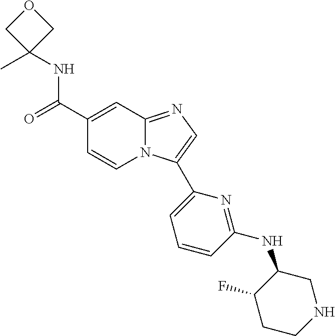 [SMILES: CC1(NC(=O)c2ccn3c(-c4cccc(N[C@H]5CNCC[C@@H]5F)n4)cnc3c2)COC1] |
| 273-7 | 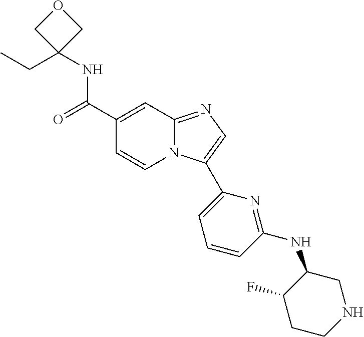 [SMILES: CCC1(NC(=O)c2ccn3c(-c4cccc(N[C@H]5CNCC[C@@H]5F)n4)cnc3c2)COC1] |
| 274-7 | 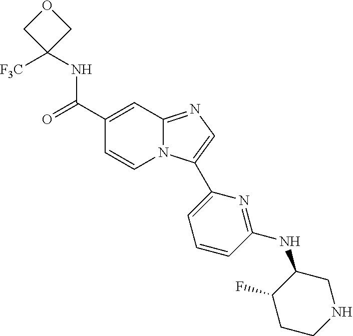 [SMILES: O=C(NC1(C(F)(F)F)COC1)c1ccn2c(-c3cccc(N[C@H]4CNCC[C@@H]4F)n3)cnc2c1] |
| 275-7 | 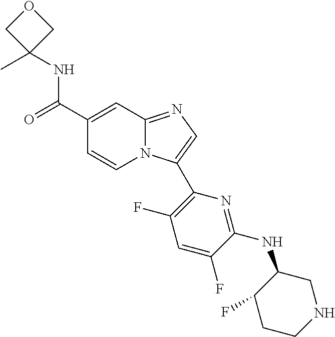 [SMILES: CC1(NC(=O)c2ccn3c(-c4nc(N[C@H]5CNCC[C@@H]5F)c(F)cc4F)cnc3c2)COC1] |
| 276-7 | 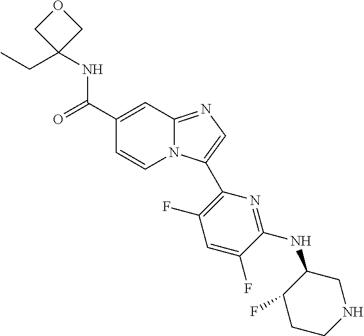 [SMILES: CCC1(NC(=O)c2ccn3c(-c4nc(N[C@H]5CNCC[C@@H]5F)c(F)cc4F)cnc3c2)COC1] |
| 277-7 | 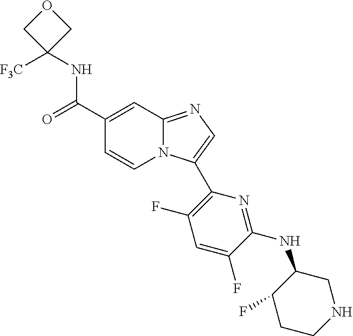 [SMILES: O=C(NC1(C(F)(F)F)COC1)c1ccn2c(-c3nc(N[C@H]4CNCC[C@@H]4F)c(F)cc3F)cnc2c1] |
| 278-7 | 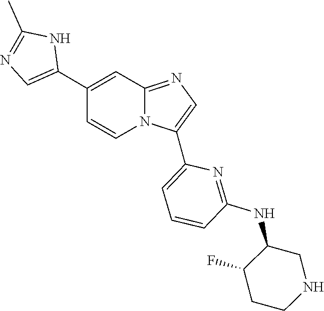 [SMILES: Cc1ncc(-c2ccn3c(-c4cccc(N[C@H]5CNCC[C@@H]5F)n4)cnc3c2)[nH]1] |
| 279-7 | 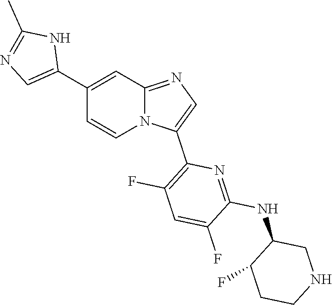 [SMILES: Cc1ncc(-c2ccn3c(-c4nc(N[C@H]5CNCC[C@@H]5F)c(F)cc4F)cnc3c2)[nH]1] |
| 280-7 | 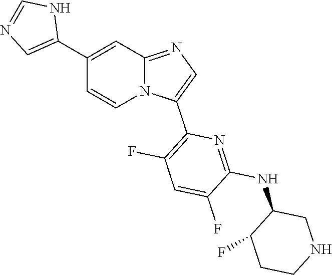 [SMILES: Fc1cc(F)c(-c2cnc3cc(-c4cnc[nH]4)ccn23)nc1N[C@H]1CNCC[C@@H]1F] |
| 281-7 | 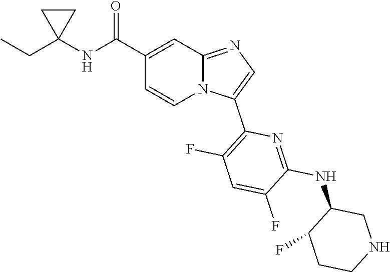 [SMILES: CCC1(NC(=O)c2ccn3c(-c4nc(N[C@H]5CNCC[C@@H]5F)c(F)cc4F)cnc3c2)CC1] |
| 282-7 | 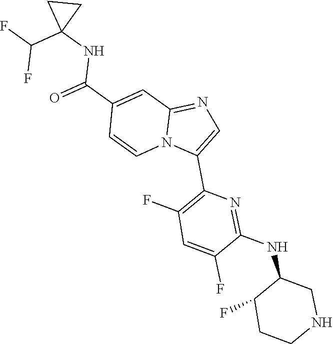 [SMILES: O=C(NC1(C(F)F)CC1)c1ccn2c(-c3nc(N[C@H]4CNCC[C@@H]4F)c(F)cc3F)cnc2c1] |
| 283-7 | 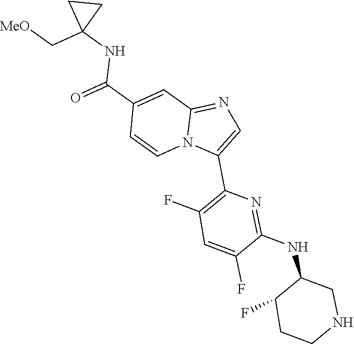 [SMILES: COCC1(NC(=O)c2ccn3c(-c4nc(N[C@H]5CNCC[C@@H]5F)c(F)cc4F)cnc3c2)CC1] |
| 284-7 | 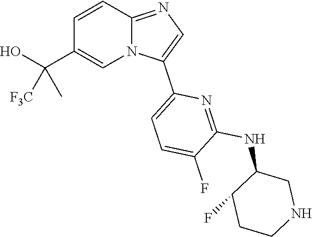 [SMILES: CC(O)(c1ccc2ncc(-c3ccc(F)c(N[C@H]4CNCC[C@@H]4F)n3)n2c1)C(F)(F)F] |
| 285-7 | 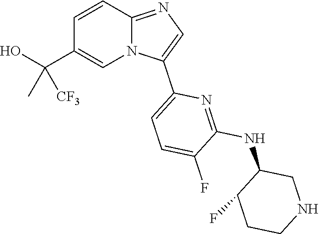 [SMILES: CC(O)(c1ccc2ncc(-c3ccc(F)c(N[C@H]4CNCC[C@@H]4F)n3)n2c1)C(F)(F)F] |
| 286-7 | 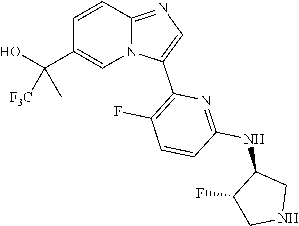 [SMILES: CC(O)(c1ccc2ncc(-c3nc(N[C@H]4CNC[C@@H]4F)ccc3F)n2c1)C(F)(F)F] |
| 287-7 | 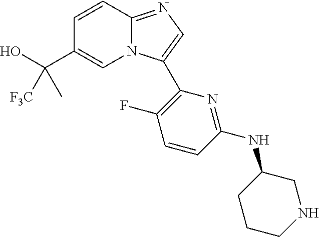 [SMILES: CC(O)(c1ccc2ncc(-c3nc(N[C@@H]4CCCNC4)ccc3F)n2c1)C(F)(F)F] |
| 288-7 | 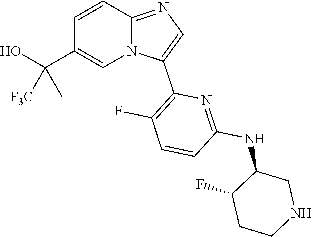 [SMILES: CC(O)(c1ccc2ncc(-c3nc(N[C@H]4CNCC[C@@H]4F)ccc3F)n2c1)C(F)(F)F] |
| 289-7 | 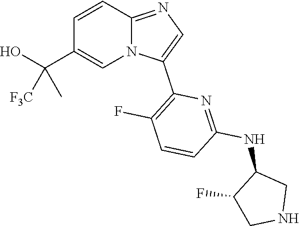 [SMILES: CC(O)(c1ccc2ncc(-c3nc(N[C@H]4CNC[C@@H]4F)ccc3F)n2c1)C(F)(F)F] |
| 290-7 | 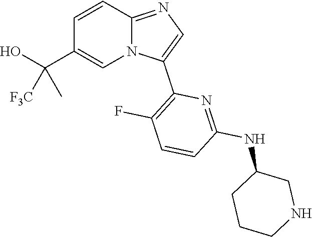 [SMILES: CC(O)(c1ccc2ncc(-c3nc(N[C@@H]4CCCNC4)ccc3F)n2c1)C(F)(F)F] |
| 291-7 | 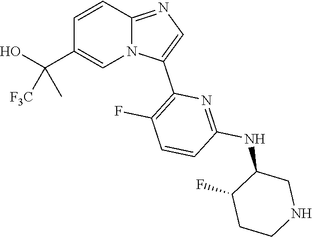 [SMILES: CC(O)(c1ccc2ncc(-c3nc(N[C@H]4CNCC[C@@H]4F)ccc3F)n2c1)C(F)(F)F] |
| 292-7 | 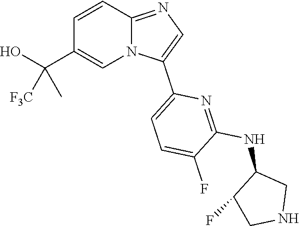 [SMILES: CC(O)(c1ccc2ncc(-c3ccc(F)c(N[C@H]4CNC[C@@H]4F)n3)n2c1)C(F)(F)F] |
| 293-7 | 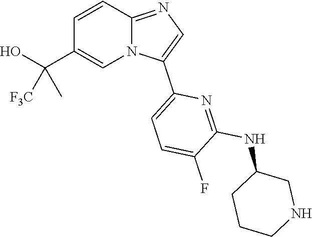 [SMILES: CC(O)(c1ccc2ncc(-c3ccc(F)c(N[C@@H]4CCCNC4)n3)n2c1)C(F)(F)F] |
| 294-7 | 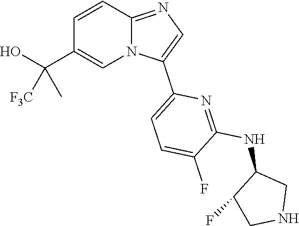 [SMILES: CC(O)(c1ccc2ncc(-c3ccc(F)c(N[C@H]4CNC[C@@H]4F)n3)n2c1)C(F)(F)F] |
| 295-7 | 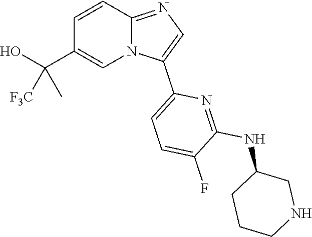 [SMILES: CC(O)(c1ccc2ncc(-c3ccc(F)c(N[C@@H]4CCCNC4)n3)n2c1)C(F)(F)F] |
| 286-7 | 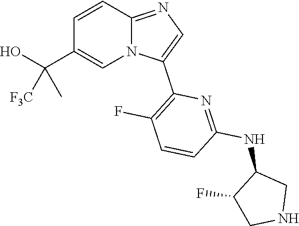 [SMILES: CC(O)(c1ccc2ncc(-c3nc(N[C@H]4CNC[C@@H]4F)ccc3F)n2c1)C(F)(F)F] |
| 287-7 | 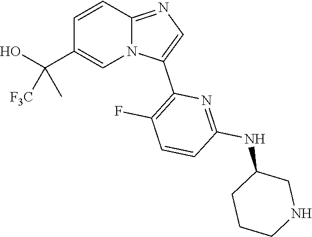 [SMILES: CC(O)(c1ccc2ncc(-c3nc(N[C@@H]4CCCNC4)ccc3F)n2c1)C(F)(F)F] |
| 288-7 | 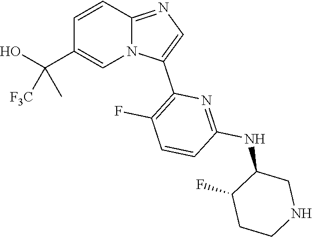 [SMILES: CC(O)(c1ccc2ncc(-c3nc(N[C@H]4CNCC[C@@H]4F)ccc3F)n2c1)C(F)(F)F] |
| 289-7 | 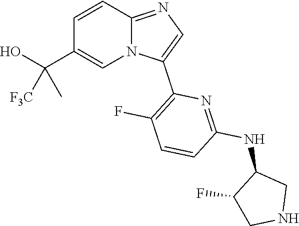 [SMILES: CC(O)(c1ccc2ncc(-c3nc(N[C@H]4CNC[C@@H]4F)ccc3F)n2c1)C(F)(F)F] |
| 290-7 | 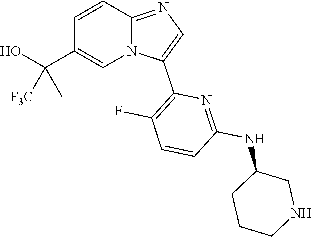 [SMILES: CC(O)(c1ccc2ncc(-c3nc(N[C@@H]4CCCNC4)ccc3F)n2c1)C(F)(F)F] |
| 291-7 | 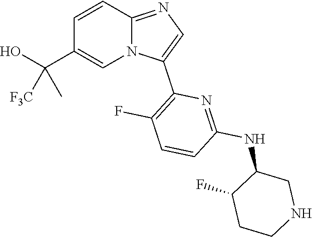 [SMILES: CC(O)(c1ccc2ncc(-c3nc(N[C@H]4CNCC[C@@H]4F)ccc3F)n2c1)C(F)(F)F] |
| 292-7 | 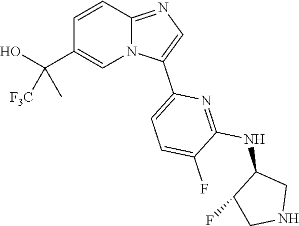 [SMILES: CC(O)(c1ccc2ncc(-c3ccc(F)c(N[C@H]4CNC[C@@H]4F)n3)n2c1)C(F)(F)F] |
| 293-7 | 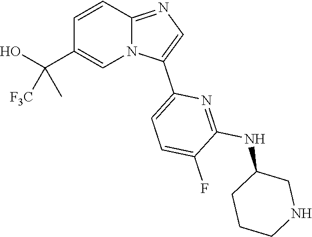 [SMILES: CC(O)(c1ccc2ncc(-c3ccc(F)c(N[C@@H]4CCCNC4)n3)n2c1)C(F)(F)F] |
| 294-7 | 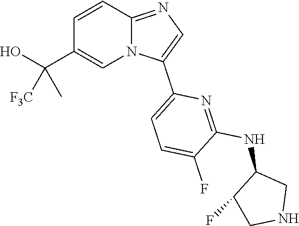 [SMILES: CC(O)(c1ccc2ncc(-c3ccc(F)c(N[C@H]4CNC[C@@H]4F)n3)n2c1)C(F)(F)F] |
| 295-7 | 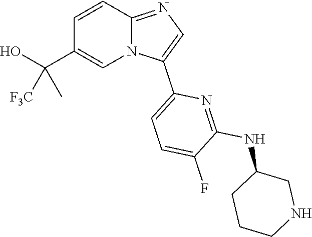 [SMILES: CC(O)(c1ccc2ncc(-c3ccc(F)c(N[C@@H]4CCCNC4)n3)n2c1)C(F)(F)F] |
| 1a-7 | 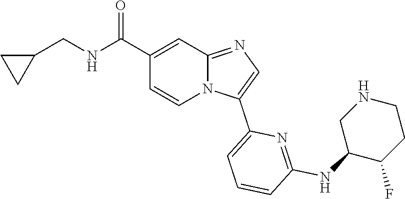 [SMILES: O=C(NCC1CC1)c1ccn2c(-c3cccc(N[C@H]4CNCC[C@@H]4F)n3)cnc2c1] |
| 2a-7 | 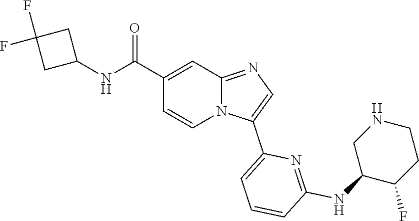 [SMILES: O=C(NC1CC(F)(F)C1)c1ccn2c(-c3cccc(N[C@H]4CNCC[C@@H]4F)n3)cnc2c1] |
| 3a-7 | 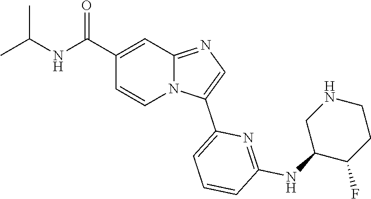 [SMILES: CC(C)NC(=O)c1ccn2c(-c3cccc(N[C@H]4CNCC[C@@H]4F)n3)cnc2c1] |
| 4a-7 | 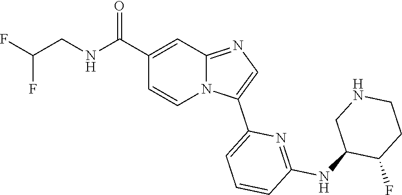 [SMILES: O=C(NCC(F)F)c1ccn2c(-c3cccc(N[C@H]4CNCC[C@@H]4F)n3)cnc2c1] |
| 5a-7 | 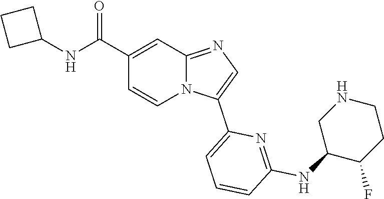 [SMILES: O=C(NC1CCC1)c1ccn2c(-c3cccc(N[C@H]4CNCC[C@@H]4F)n3)cnc2c1] |
| 6a-7 | 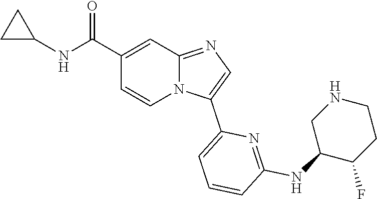 [SMILES: O=C(NC1CC1)c1ccn2c(-c3cccc(N[C@H]4CNCC[C@@H]4F)n3)cnc2c1] |
| 7a-7 | 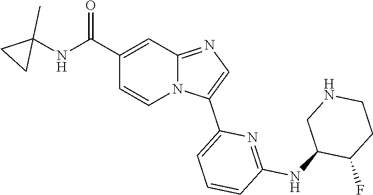 [SMILES: CC1(NC(=O)c2ccn3c(-c4cccc(N[C@H]5CNCC[C@@H]5F)n4)cnc3c2)CC1] |
| 8a-7 | 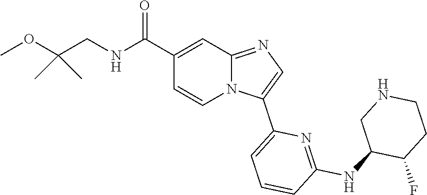 [SMILES: COC(C)(C)CNC(=O)c1ccn2c(-c3cccc(N[C@H]4CNCC[C@@H]4F)n3)cnc2c1] |
| 9a-7 | 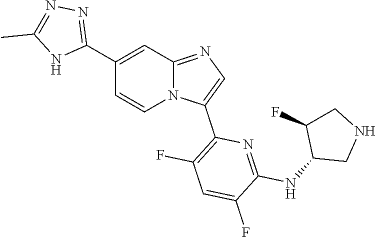 [SMILES: Cc1nnc(-c2ccn3c(-c4nc(N[C@H]5CNC[C@@H]5F)c(F)cc4F)cnc3c2)[nH]1] |
| 10a-7 | 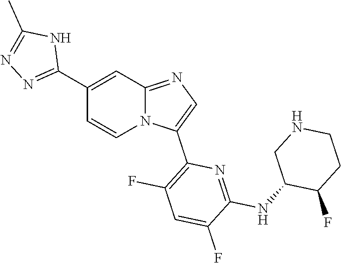 [SMILES: Cc1nnc(-c2ccn3c(-c4nc(N[C@@H]5CNCC[C@H]5F)c(F)cc4F)cnc3c2)[nH]1] |
| 11a-7 | 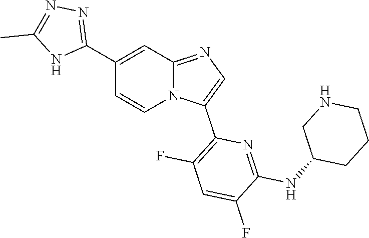 [SMILES: Cc1nnc(-c2ccn3c(-c4nc(N[C@H]5CCCNC5)c(F)cc4F)cnc3c2)[nH]1] |
| 12a-7 | 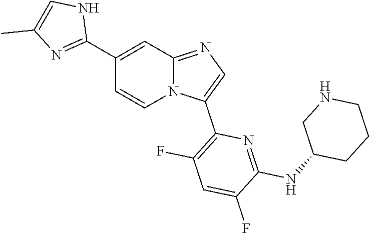 [SMILES: Cc1c[nH]c(-c2ccn3c(-c4nc(N[C@H]5CCCNC5)c(F)cc4F)cnc3c2)n1] |
| 13a-7 | 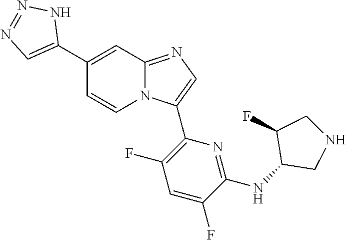 [SMILES: Fc1cc(F)c(-c2cnc3cc(-c4cnn[nH]4)ccn23)nc1N[C@H]1CNC[C@@H]1F] |
| 14a-7 | 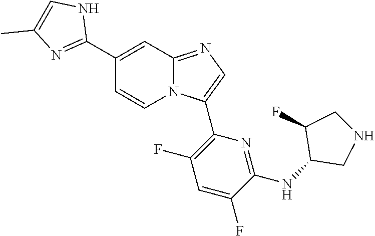 [SMILES: Cc1c[nH]c(-c2ccn3c(-c4nc(N[C@H]5CNC[C@@H]5F)c(F)cc4F)cnc3c2)n1] |
| 15a-7 | 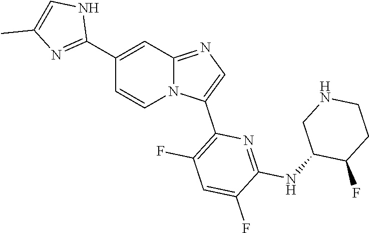 [SMILES: Cc1c[nH]c(-c2ccn3c(-c4nc(N[C@@H]5CNCC[C@H]5F)c(F)cc4F)cnc3c2)n1] |
| 16a-7 | 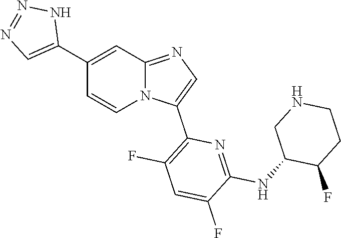 [SMILES: Fc1cc(F)c(-c2cnc3cc(-c4cnn[nH]4)ccn23)nc1N[C@@H]1CNCC[C@H]1F] |
| 17a-7 | 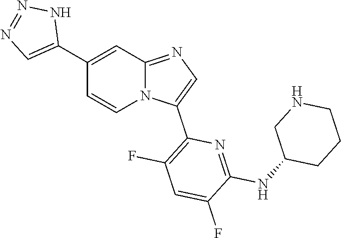 [SMILES: Fc1cc(F)c(-c2cnc3cc(-c4cnn[nH]4)ccn23)nc1N[C@H]1CCCNC1] |
| 18a-7 | 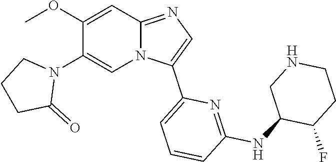 [SMILES: COc1cc2ncc(-c3cccc(N[C@H]4CNCC[C@@H]4F)n3)n2cc1N1CCCC1=O] |
| 19a-7 | 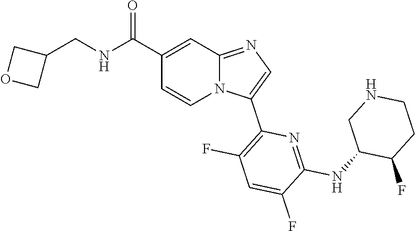 [SMILES: O=C(NCC1COC1)c1ccn2c(-c3nc(N[C@@H]4CNCC[C@H]4F)c(F)cc3F)cnc2c1] |
| 20a-7 | 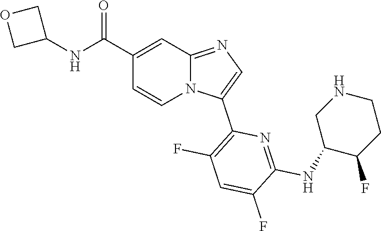 [SMILES: O=C(NC1COC1)c1ccn2c(-c3nc(N[C@@H]4CNCC[C@H]4F)c(F)cc3F)cnc2c1] |
| 21a-7 | 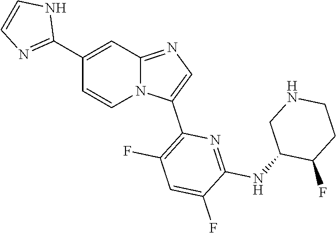 [SMILES: Fc1cc(F)c(-c2cnc3cc(-c4ncc[nH]4)ccn23)nc1N[C@@H]1CNCC[C@H]1F] |
| 22a-7 | 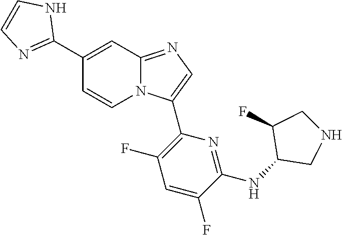 [SMILES: Fc1cc(F)c(-c2cnc3cc(-c4ncc[nH]4)ccn23)nc1N[C@H]1CNC[C@@H]1F] |
| 23a-7 | 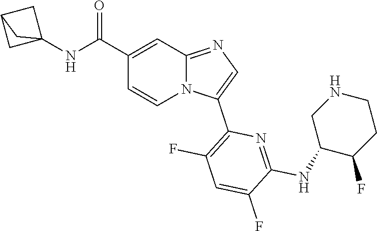 [SMILES: O=C(NC12CC(C1)C2)c1ccn2c(-c3nc(N[C@@H]4CNCC[C@H]4F)c(F)cc3F)cnc2c1] |
| 24a-7 | 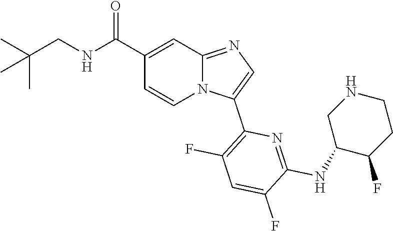 [SMILES: CC(C)(C)CNC(=O)c1ccn2c(-c3nc(N[C@@H]4CNCC[C@H]4F)c(F)cc3F)cnc2c1] |
| 25a-7 | 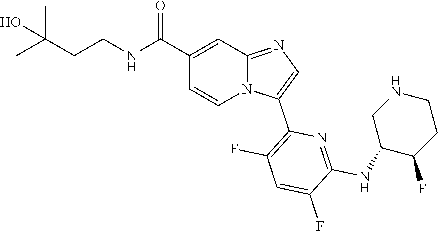 [SMILES: CC(C)(O)CCNC(=O)c1ccn2c(-c3nc(N[C@@H]4CNCC[C@H]4F)c(F)cc3F)cnc2c1] |
| 26a-7 | 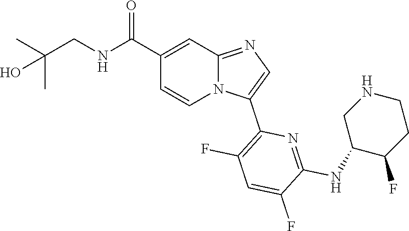 [SMILES: CC(C)(O)CNC(=O)c1ccn2c(-c3nc(N[C@@H]4CNCC[C@H]4F)c(F)cc3F)cnc2c1] |
| 27a-7 | 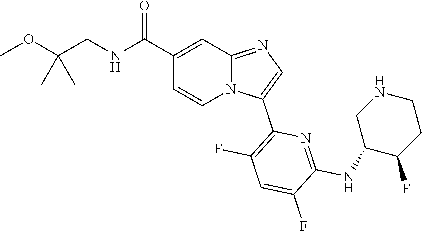 [SMILES: COC(C)(C)CNC(=O)c1ccn2c(-c3nc(N[C@@H]4CNCC[C@H]4F)c(F)cc3F)cnc2c1] |
| 28a-7 | 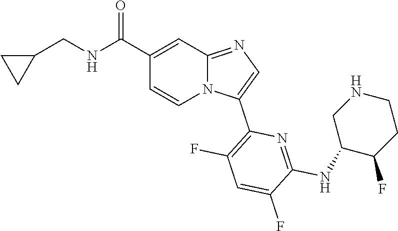 [SMILES: O=C(NCC1CC1)c1ccn2c(-c3nc(N[C@@H]4CNCC[C@H]4F)c(F)cc3F)cnc2c1] |
| 29a-7 | 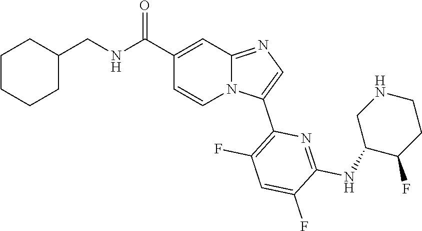 [SMILES: O=C(NCC1CCCCC1)c1ccn2c(-c3nc(N[C@@H]4CNCC[C@H]4F)c(F)cc3F)cnc2c1] |
| 30a-7 | 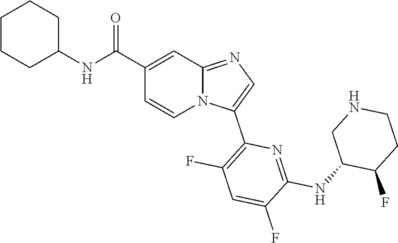 [SMILES: O=C(NC1CCCCC1)c1ccn2c(-c3nc(N[C@@H]4CNCC[C@H]4F)c(F)cc3F)cnc2c1] |
| 31a-7 | 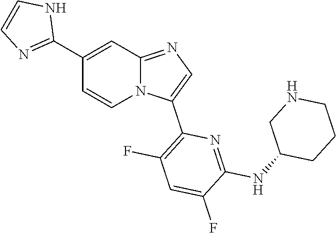 [SMILES: Fc1cc(F)c(-c2cnc3cc(-c4ncc[nH]4)ccn23)nc1N[C@H]1CCCNC1] |
| 32a-7 | 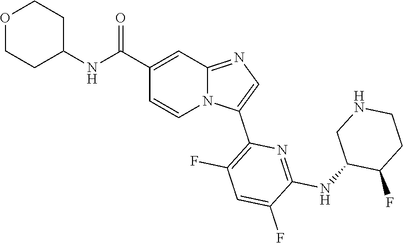 [SMILES: O=C(NC1CCOCC1)c1ccn2c(-c3nc(N[C@@H]4CNCC[C@H]4F)c(F)cc3F)cnc2c1] |
| 33a-7 | 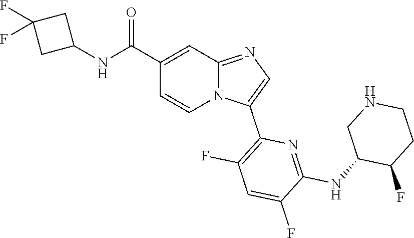 [SMILES: O=C(NC1CC(F)(F)C1)c1ccn2c(-c3nc(N[C@@H]4CNCC[C@H]4F)c(F)cc3F)cnc2c1] |
| 34a-7 | 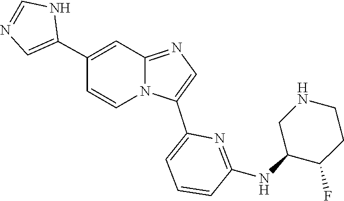 [SMILES: F[C@H]1CCNC[C@@H]1Nc1cccc(-c2cnc3cc(-c4cnc[nH]4)ccn23)n1] |
| 35a-7 | 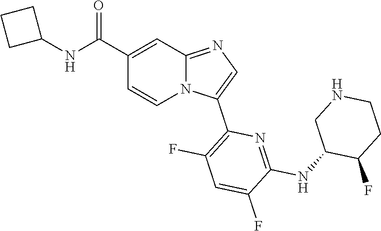 [SMILES: O=C(NC1CCC1)c1ccn2c(-c3nc(N[C@@H]4CNCC[C@H]4F)c(F)cc3F)cnc2c1] |
| 36a-7 | 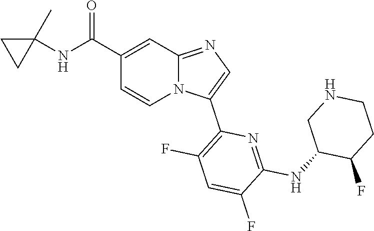 [SMILES: CC1(NC(=O)c2ccn3c(-c4nc(N[C@@H]5CNCC[C@H]5F)c(F)cc4F)cnc3c2)CC1] |
| 37a-7 | 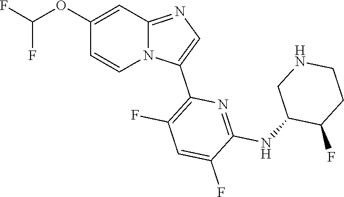 [SMILES: Fc1cc(F)c(-c2cnc3cc(OC(F)F)ccn23)nc1N[C@@H]1CNCC[C@H]1F] |
| 38a-7 | 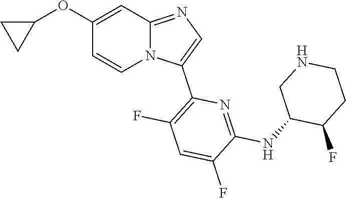 [SMILES: Fc1cc(F)c(-c2cnc3cc(OC4CC4)ccn23)nc1N[C@@H]1CNCC[C@H]1F] |
| 39a-7 | 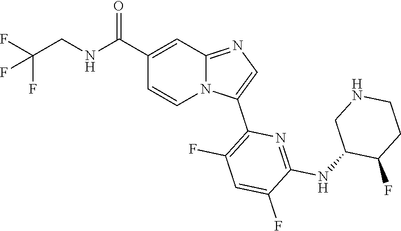 [SMILES: O=C(NCC(F)(F)F)c1ccn2c(-c3nc(N[C@@H]4CNCC[C@H]4F)c(F)cc3F)cnc2c1] |
| 40a-7 | 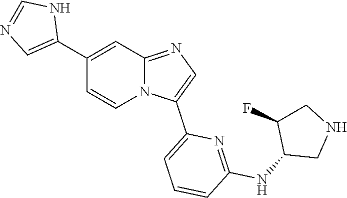 [SMILES: F[C@H]1CNC[C@@H]1Nc1cccc(-c2cnc3cc(-c4cnc[nH]4)ccn23)n1] |
| 41a-7 | 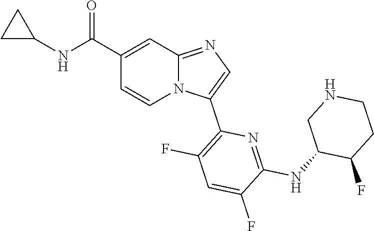 [SMILES: O=C(NC1CC1)c1ccn2c(-c3nc(N[C@@H]4CNCC[C@H]4F)c(F)cc3F)cnc2c1] |
| 42a-7 | 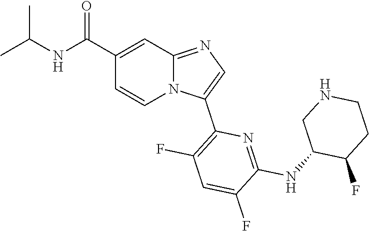 [SMILES: CC(C)NC(=O)c1ccn2c(-c3nc(N[C@@H]4CNCC[C@H]4F)c(F)cc3F)cnc2c1] |
| 43a-7 | 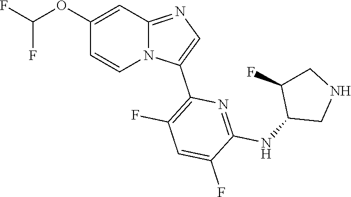 [SMILES: Fc1cc(F)c(-c2cnc3cc(OC(F)F)ccn23)nc1N[C@H]1CNC[C@@H]1F] |
| 44a-7 | 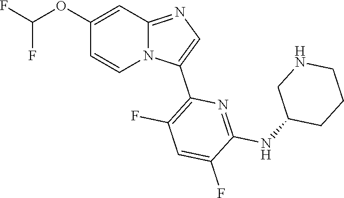 [SMILES: Fc1cc(F)c(-c2cnc3cc(OC(F)F)ccn23)nc1N[C@H]1CCCNC1] |
| 45a-7 | 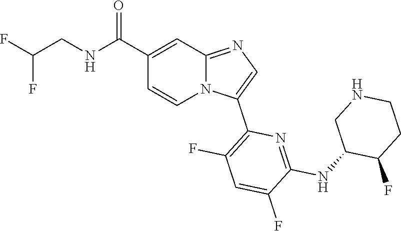 [SMILES: O=C(NCC(F)F)c1ccn2c(-c3nc(N[C@@H]4CNCC[C@H]4F)c(F)cc3F)cnc2c1] |
| 46a-7 | 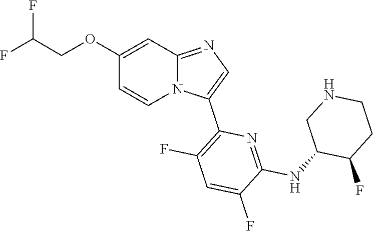 [SMILES: Fc1cc(F)c(-c2cnc3cc(OCC(F)F)ccn23)nc1N[C@@H]1CNCC[C@H]1F] |
| 47a-7 | 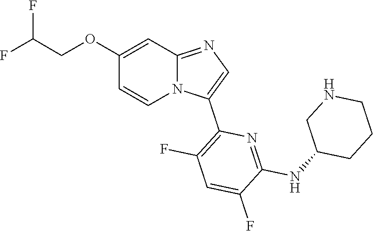 [SMILES: Fc1cc(F)c(-c2cnc3cc(OCC(F)F)ccn23)nc1N[C@H]1CCCNC1] |
| 48a-7 | 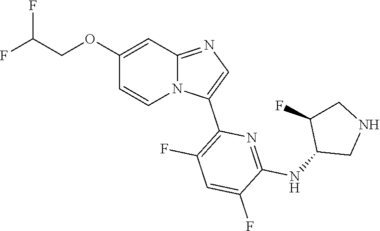 [SMILES: Fc1cc(F)c(-c2cnc3cc(OCC(F)F)ccn23)nc1N[C@H]1CNC[C@@H]1F] |
| 49a-7 | 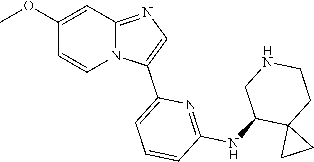 [SMILES: COc1ccn2c(-c3cccc(N[C@H]4CNCCC45CC5)n3)cnc2c1] |
| 50a-7 | 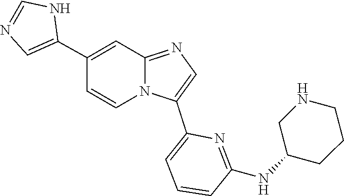 [SMILES: c1cc(N[C@H]2CCCNC2)nc(-c2cnc3cc(-c4cnc[nH]4)ccn23)c1] |
| 51a-7 | 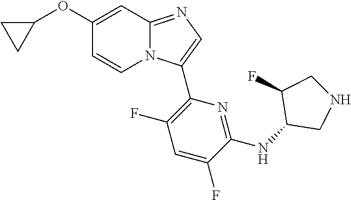 [SMILES: Fc1cc(F)c(-c2cnc3cc(OC4CC4)ccn23)nc1N[C@H]1CNC[C@@H]1F] |
| 52a-7 | 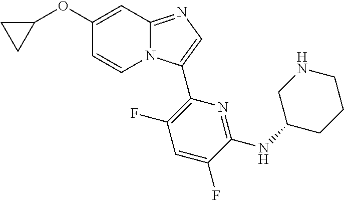 [SMILES: Fc1cc(F)c(-c2cnc3cc(OC4CC4)ccn23)nc1N[C@H]1CCCNC1] |
| 53a-7 | 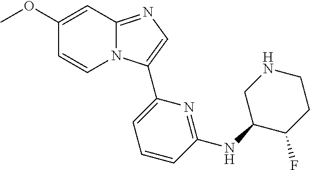 [SMILES: COc1ccn2c(-c3cccc(N[C@H]4CNCC[C@@H]4F)n3)cnc2c1] |
| 54a-7 | 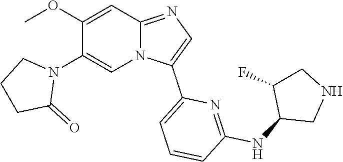 [SMILES: COc1cc2ncc(-c3cccc(N[C@@H]4CNC[C@H]4F)n3)n2cc1N1CCCC1=O] |
| 55a-7 | 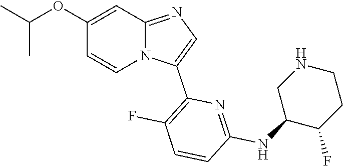 [SMILES: CC(C)Oc1ccn2c(-c3nc(N[C@H]4CNCC[C@@H]4F)ccc3F)cnc2c1] |
| 56a-7 | 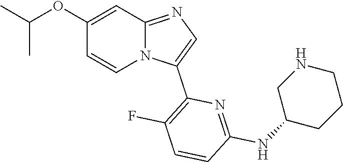 [SMILES: CC(C)Oc1ccn2c(-c3nc(N[C@H]4CCCNC4)ccc3F)cnc2c1] |
| 57a-7 | 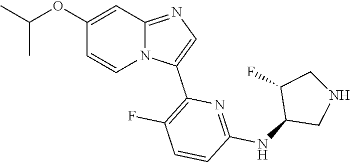 [SMILES: CC(C)Oc1ccn2c(-c3nc(N[C@@H]4CNC[C@H]4F)ccc3F)cnc2c1] |
| 58a-7 | 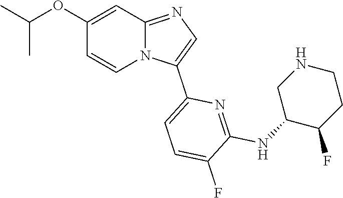 [SMILES: CC(C)Oc1ccn2c(-c3ccc(F)c(N[C@@H]4CNCC[C@H]4F)n3)cnc2c1] |
| 59a-7 | 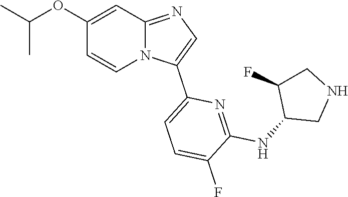 [SMILES: CC(C)Oc1ccn2c(-c3ccc(F)c(N[C@H]4CNC[C@@H]4F)n3)cnc2c1] |
| 60a-7 | 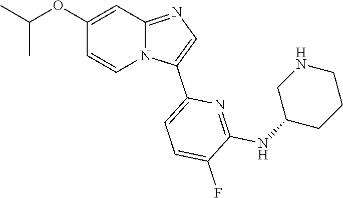 [SMILES: CC(C)Oc1ccn2c(-c3ccc(F)c(N[C@H]4CCCNC4)n3)cnc2c1] |
| 61a-7 | 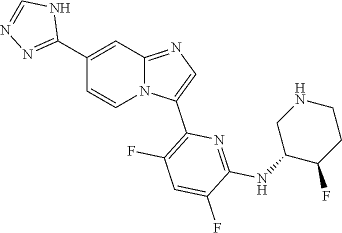 [SMILES: Fc1cc(F)c(-c2cnc3cc(-c4nnc[nH]4)ccn23)nc1N[C@@H]1CNCC[C@H]1F] |
| 62a-7 | 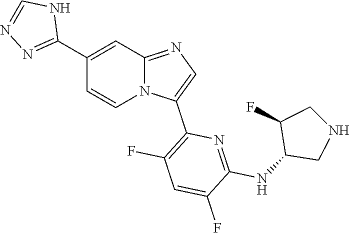 [SMILES: Fc1cc(F)c(-c2cnc3cc(-c4nnc[nH]4)ccn23)nc1N[C@H]1CNC[C@@H]1F] |
| 63a-7 | 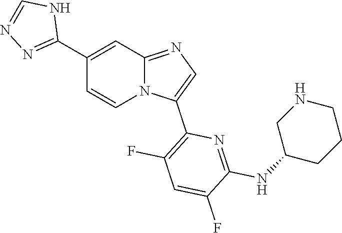 [SMILES: Fc1cc(F)c(-c2cnc3cc(-c4nnc[nH]4)ccn23)nc1N[C@H]1CCCNC1] |
| 64a-7 | 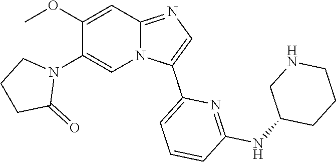 [SMILES: COc1cc2ncc(-c3cccc(N[C@H]4CCCNC4)n3)n2cc1N1CCCC1=O] |
| 65a-7 | 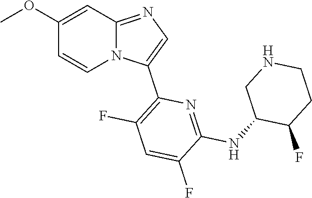 [SMILES: COc1ccn2c(-c3nc(N[C@@H]4CNCC[C@H]4F)c(F)cc3F)cnc2c1] |
| 66a-7 | 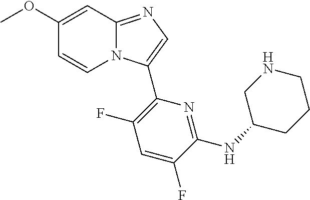 [SMILES: COc1ccn2c(-c3nc(N[C@H]4CCCNC4)c(F)cc3F)cnc2c1] |
| 67a-7 | 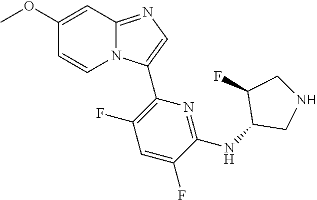 [SMILES: COc1ccn2c(-c3nc(N[C@H]4CNC[C@@H]4F)c(F)cc3F)cnc2c1] |
| 68a-7 | 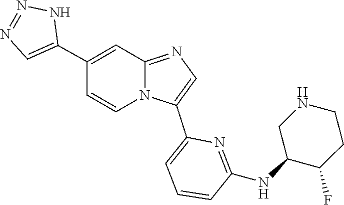 [SMILES: F[C@H]1CCNC[C@@H]1Nc1cccc(-c2cnc3cc(-c4cnn[nH]4)ccn23)n1] |
| 69a-7 | 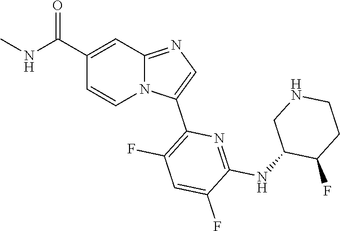 [SMILES: CNC(=O)c1ccn2c(-c3nc(N[C@@H]4CNCC[C@H]4F)c(F)cc3F)cnc2c1] |
| 70a-7 | 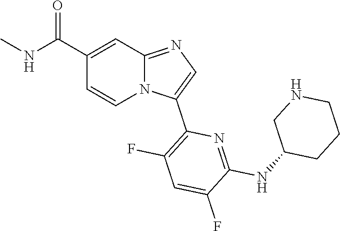 [SMILES: CNC(=O)c1ccn2c(-c3nc(N[C@H]4CCCNC4)c(F)cc3F)cnc2c1] |
| 71a-7 | 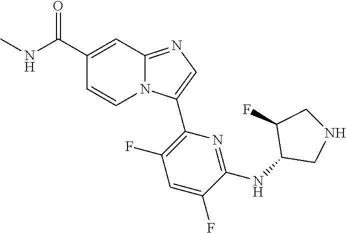 [SMILES: CNC(=O)c1ccn2c(-c3nc(N[C@H]4CNC[C@@H]4F)c(F)cc3F)cnc2c1] |
| 72a-7 | 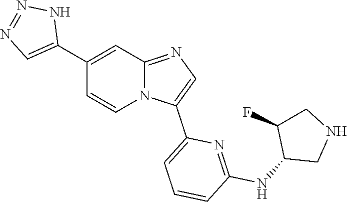 [SMILES: F[C@H]1CNC[C@@H]1Nc1cccc(-c2cnc3cc(-c4cnn[nH]4)ccn23)n1] |
| 73a-7 | 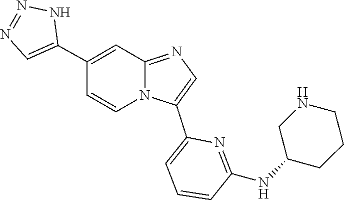 [SMILES: c1cc(N[C@H]2CCCNC2)nc(-c2cnc3cc(-c4cnn[nH]4)ccn23)c1] |
| 74a-7 | 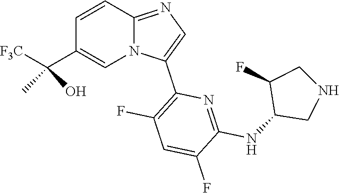 [SMILES: C[C@@](O)(c1ccc2ncc(-c3nc(N[C@H]4CNC[C@@H]4F)c(F)cc3F)n2c1)C(F)(F)F] |
| 75a-7 | 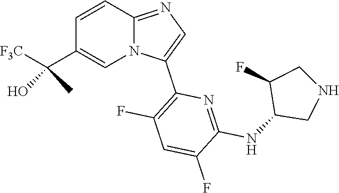 [SMILES: C[C@](O)(c1ccc2ncc(-c3nc(N[C@H]4CNC[C@@H]4F)c(F)cc3F)n2c1)C(F)(F)F] |
| 76a-7 | 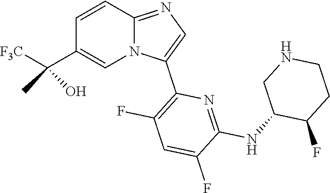 [SMILES: C[C@](O)(c1ccc2ncc(-c3nc(N[C@@H]4CNCC[C@H]4F)c(F)cc3F)n2c1)C(F)(F)F] |
| 77a-7 | 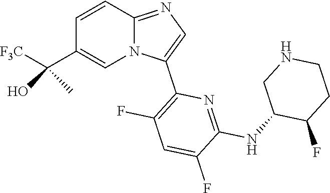 [SMILES: C[C@@](O)(c1ccc2ncc(-c3nc(N[C@@H]4CNCC[C@H]4F)c(F)cc3F)n2c1)C(F)(F)F] |
| 78a-7 | 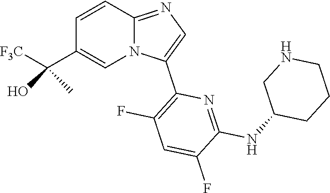 [SMILES: C[C@@](O)(c1ccc2ncc(-c3nc(N[C@H]4CCCNC4)c(F)cc3F)n2c1)C(F)(F)F] |
| 79a-7 | 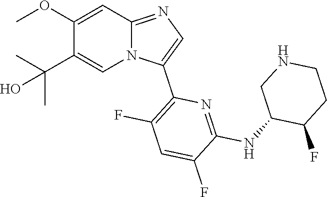 [SMILES: COc1cc2ncc(-c3nc(N[C@@H]4CNCC[C@H]4F)c(F)cc3F)n2cc1C(C)(C)O] |

In another embodiment, the compound is a compound of Formula (VI-5003):

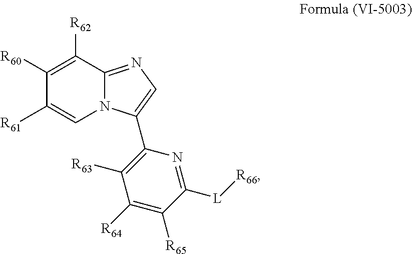

or a salt, ester, solvate, optical isomer, geometric isomer, or salt of an isomer thereof; wherein:
R₆₀ is selected from H, halogen, hydroxy, C₁-C₇ alkyl, C₂-C₇ alkenyl, C₂-C₇ alkynyl, or C₁-C₆ alkoxy, wherein the C₁-C₇ alkyl, C₂-C₇ alkenyl, C₂-C₇ alkynyl, or C₁-C₆ alkoxy is optionally substituted with one or more of halogen, hydroxy, methanoyl (—COH), carboxy (—CO₂H), nitro (—NO₂), cyano (—CN), ethynyl (—CCH), sulfo (—SO₃H), methyl, ethyl, or morpholinyl;
R₆₁ is selected from H, halogen, hydroxy, —CN, methanoyl (—COH), carboxy (—CO₂H), C₁-C₇ alkyl, C₂-C₇ alkenyl, C₂-C₇ alkynyl, C₁-C₆ alkoxy, cycloalkyl, heterocyclyl, aryl, or heteroaryl, wherein the methanoyl (—COH), carboxy (—CO₂H), C₁-C₇ alkyl, C₂-C₇ alkenyl, C₂-C₇ alkynyl, C₁-C₆ alkoxy, cycloalkyl, heterocyclyl, aryl, or heteroaryl is optionally substituted with one or more of halogen, hydroxy, methanoyl (—COH), carboxy (—CO₂H), nitro (—NO₂), —NH₂, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CON(CH₃)₂, C₁-C₃ alkyl, C₁-C₃ perfluorinated alkyl, or C₁-C₃ alkoxy;
R₆₂ is selected from H, halogen, hydroxy, C₁-C₃ alkyl, C₂-C₃ alkenyl, C₂-C₃ alkynyl, or C₁-C₂ alkoxy, wherein the C₁-C₃ alkyl, C₂-C₃ alkenyl, C₂-C₃ alkynyl, or C₁-C₂ alkoxy is optionally substituted with one or more of halogen, hydroxy, methanoyl (—COH), carboxy (—CO₂H), cyano (—CN), ethynyl (—CCH), sulfo (—SO₃H), methyl, or ethyl;
R₆₃ is selected from H, halogen, hydroxy, methanoyl (—COH), carboxy (—CO₂H), nitro (—NO₂), cyano (—CN), sulfo (—SO₃H), C₁-C₄ alkyl, C₂-C₄ alkenyl, C₂-C₄ alkynyl, C₁-C₃ alkoxy, or —O-aryl, wherein the C₁-C₄ alkyl, C₂-C₄ alkenyl, C₂-C₄ alkynyl, C₁-C₃ alkoxy, or —O-aryl is optionally substituted with one or more of halogen, hydroxy, methanoyl (—COH), carboxy (—CO₂H), nitro (—NO₂), cyano (—CN), ethynyl (—CCH), sulfo (—SO₃H), methyl, or ethyl;
R₆₄ is selected from H, halogen, hydroxy, methanoyl (—COH), carboxy (—CO₂H), nitro (—NO₂), cyano (—CN), sulfo (—SO₃H), C₁-C₄ alkyl, C₂-C₄ alkenyl, C₂-C₄ alkynyl, or C₁-C₃ alkoxy, which C₁-C₄ alkyl, C₂-C₄ alkenyl, C₂-C₄ alkynyl, or C₁-C₃ alkoxy is optionally substituted with one or more of halogen, hydroxy, methanoyl (—COH), carboxy (—CO₂H), nitro (—NO₂), cyano (—CN), ethynyl (—CCH), sulfo (—SO₃H), methyl, or ethyl;
R₆₅ is selected from H, halogen, hydroxy, methanoyl (—COH), carboxy (—CO₂H), nitro (—NO₂), cyano (—CN), sulfo (—SO₃H), C₁-C₄ alkyl, C₂-C₄ alkenyl, C₂-C₄ alkynyl, or C₁-C₃ alkoxy, wherein the C₁-C₄ alkyl, C₂-C₄ alkenyl, C₂-C₄ alkynyl, or C₁-C₃ alkoxy is optionally substituted with one or more of halogen, hydroxy, methanoyl (—COH), carboxy (—CO₂H), nitro (—NO₂), cyano (—CN), ethynyl (—CCH), sulfo (—SO₃H), methyl, or ethyl;
R₆₆ is

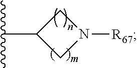

R₆₇ is selected from H, C₁-C₄ alkyl, C₂-C₄ alkenyl, C₂-C₄ alkynyl, methanoyl (—COH), ethanoyl (—COCH₃), benzoyl (—COC₆H₅), toluoyl, carboxy (—CO₂H), nitro (—NO₂), cyano (—CN), or —COCH₂CN;
L is selected from —NH—, —N(CH₃)—, —N(CH₂CH₃)—, —N(CH₂CH₂CH₃)—, —N[CH(CH₃)₂]—, or —O—;
n is 0, 1, 2, 3, 4, or 5; and
m is 0, 1, 2, 3, 4, or 5;
   with the proviso that n+m is at least 1.

In one embodiment, the compound of Formula (VI-5003) is selected from Compound 1-3 to 68-3.

| # | Structure |
|---|---|
| 1-3 | 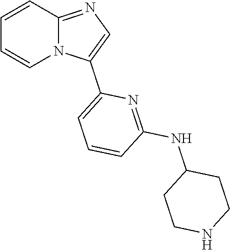 [SMILES: c1cc(NC2CCNCC2)nc(-c2cnc3ccccn23)c1] |
| 2-3 | 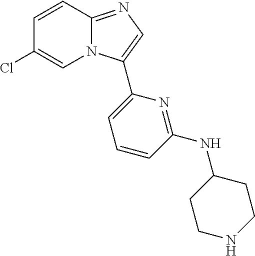 [SMILES: Clc1ccc2ncc(-c3cccc(NC4CCNCC4)n3)n2c1] |
| 3-3 | 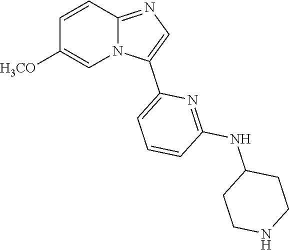 [SMILES: COc1ccc2ncc(-c3cccc(NC4CCNCC4)n3)n2c1] |
| 4-3 | 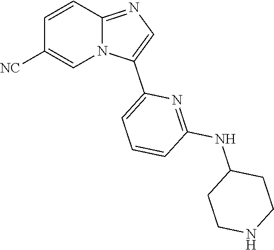 [SMILES: N#Cc1ccc2ncc(-c3cccc(NC4CCNCC4)n3)n2c1] |
| 5-3 | 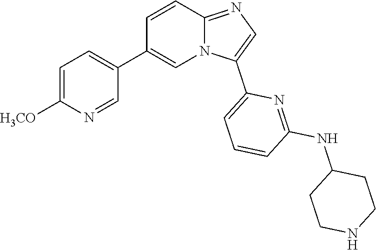 [SMILES: COc1ccc(-c2ccc3ncc(-c4cccc(NC5CCNCC5)n4)n3c2)cn1] |
| 6-3 | 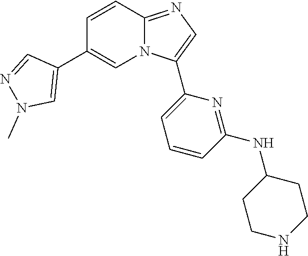 [SMILES: Cn1cc(-c2ccc3ncc(-c4cccc(NC5CCNCC5)n4)n3c2)cn1] |
| 7-3 | 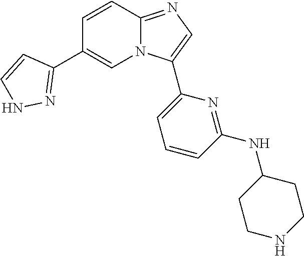 [SMILES: c1cc(NC2CCNCC2)nc(-c2cnc3ccc(-c4cc[nH]n4)cn23)c1] |
| 8-3 | 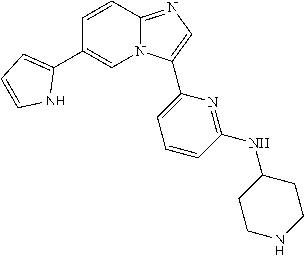 [SMILES: c1cc(NC2CCNCC2)nc(-c2cnc3ccc(-c4ccc[nH]4)cn23)c1] |
| 9-3 | 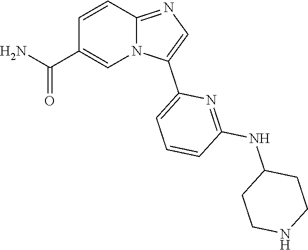 [SMILES: NC(=O)c1ccc2ncc(-c3cccc(NC4CCNCC4)n3)n2c1] |
| 10-3 | 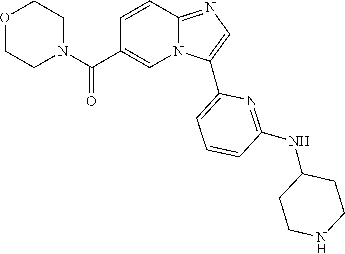 [SMILES: O=C(c1ccc2ncc(-c3cccc(NC4CCNCC4)n3)n2c1)N1CCOCC1] |
| 11-3 | 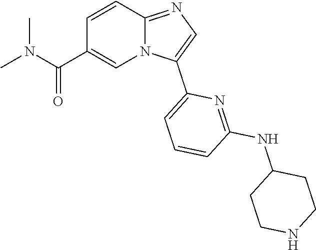 [SMILES: CN(C)C(=O)c1ccc2ncc(-c3cccc(NC4CCNCC4)n3)n2c1] |
| 12-3 | 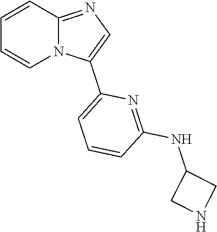 [SMILES: c1cc(NC2CNC2)nc(-c2cnc3ccccn23)c1] |
| 13-3 | 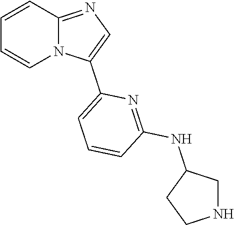 [SMILES: c1cc(NC2CCNC2)nc(-c2cnc3ccccn23)c1] |
| 14-3 | 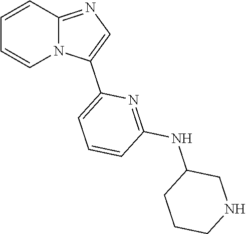 [SMILES: c1cc(NC2CCCNC2)nc(-c2cnc3ccccn23)c1] |
| 15-3 | 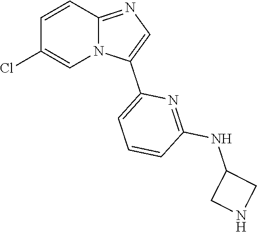 [SMILES: Clc1ccc2ncc(-c3cccc(NC4CNC4)n3)n2c1] |
| 16-3 | 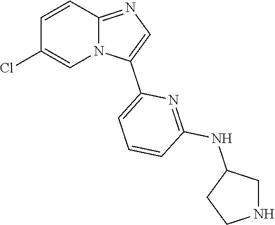 [SMILES: Clc1ccc2ncc(-c3cccc(NC4CCNC4)n3)n2c1] |
| 17-3 | 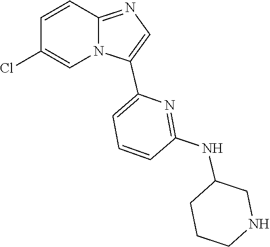 [SMILES: Clc1ccc2ncc(-c3cccc(NC4CCCNC4)n3)n2c1] |
| 18-3 | 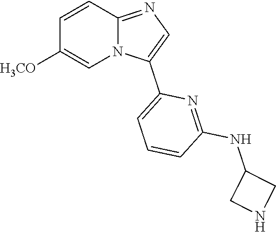 [SMILES: COc1ccc2ncc(-c3cccc(NC4CNC4)n3)n2c1] |
| 19-3 | 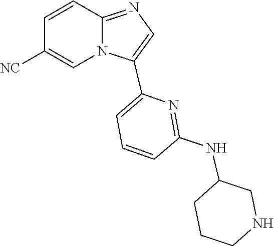 [SMILES: N#Cc1ccc2ncc(-c3cccc(NC4CCCNC4)n3)n2c1] |
| 20-3 | 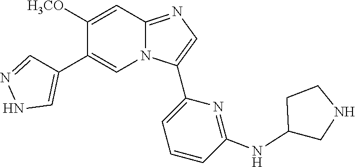 [SMILES: COc1cc2ncc(-c3cccc(NC4CCNC4)n3)n2cc1-c1cn[nH]c1] |
| 21-3 | 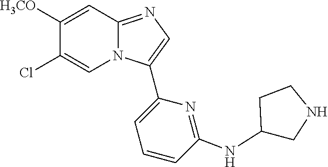 [SMILES: COc1cc2ncc(-c3cccc(NC4CCNC4)n3)n2cc1Cl] |
| 22-3 | 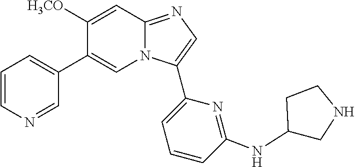 [SMILES: COc1cc2ncc(-c3cccc(NC4CCNC4)n3)n2cc1-c1cccnc1] |
| 23-3 | 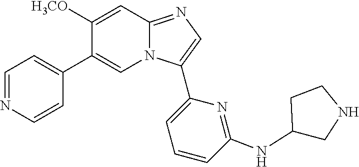 [SMILES: COc1cc2ncc(-c3cccc(NC4CCNC4)n3)n2cc1-c1ccncc1] |
| 24-3 | 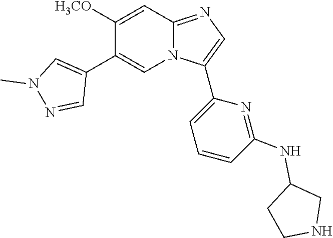 [SMILES: COc1cc2ncc(-c3cccc(NC4CCNC4)n3)n2cc1-c1cnn(C)c1] |
| 25-3 | 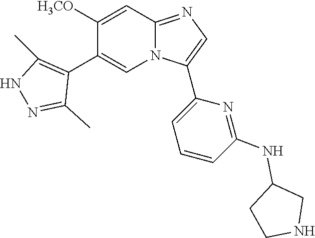 [SMILES: COc1cc2ncc(-c3cccc(NC4CCNC4)n3)n2cc1-c1c(C)n[nH]c1C] |
| 26-3 | 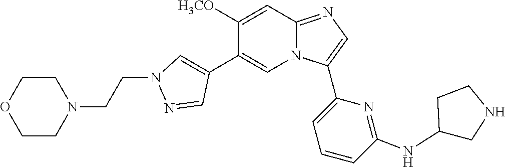 [SMILES: COc1cc2ncc(-c3cccc(NC4CCNC4)n3)n2cc1-c1cnn(CCN2CCOCC2)c1] |
| 27-3 | 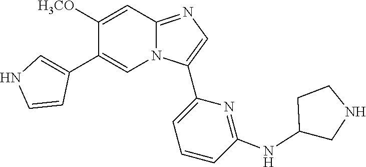 [SMILES: COc1cc2ncc(-c3cccc(NC4CCNC4)n3)n2cc1-c1cc[nH]c1] |
| 28-3 | 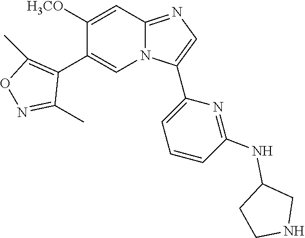 [SMILES: COc1cc2ncc(-c3cccc(NC4CCNC4)n3)n2cc1-c1c(C)noc1C] |
| 29-3 | 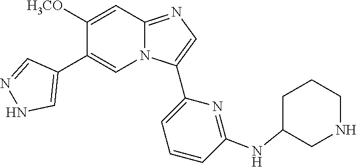 [SMILES: COc1cc2ncc(-c3cccc(NC4CCCNC4)n3)n2cc1-c1cn[nH]c1] |
| 30-3 | 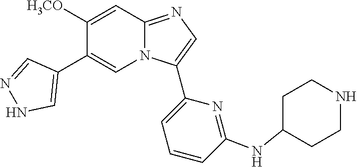 [SMILES: COc1cc2ncc(-c3cccc(NC4CCNCC4)n3)n2cc1-c1cn[nH]c1] |
| 31-3 | 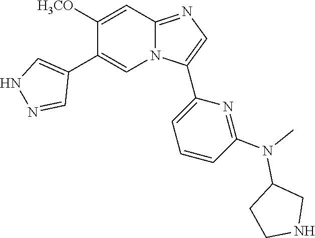 [SMILES: COc1cc2ncc(-c3cccc(N(C)C4CCNC4)n3)n2cc1-c1cn[nH]c1] |
| 32-3 | 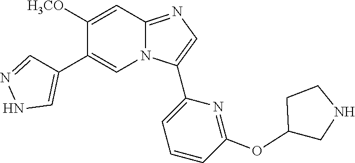 [SMILES: COc1cc2ncc(-c3cccc(OC4CCNC4)n3)n2cc1-c1cn[nH]c1] |
| 33-3 | 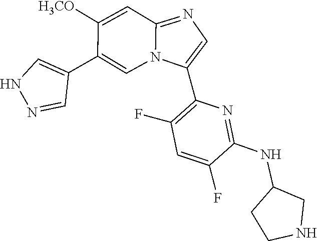 [SMILES: COc1cc2ncc(-c3nc(NC4CCNC4)c(F)cc3F)n2cc1-c1cn[nH]c1] |
| 34-3 | 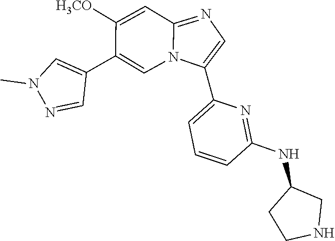 [SMILES: COc1cc2ncc(-c3cccc(N[C@@H]4CCNC4)n3)n2cc1-c1cnn(C)c1] |
| 35-3 | 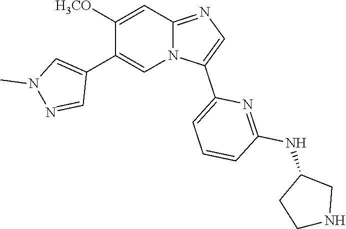 [SMILES: COc1cc2ncc(-c3cccc(N[C@H]4CCNC4)n3)n2cc1-c1cnn(C)c1] |
| 36-3 | 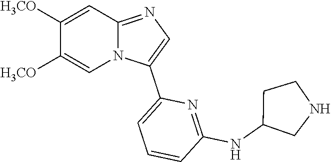 [SMILES: COc1cc2ncc(-c3cccc(NC4CCNC4)n3)n2cc1OC] |
| 37-3 | 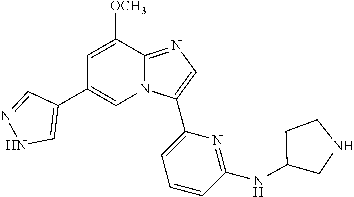 [SMILES: COc1cc(-c2cn[nH]c2)cn2c(-c3cccc(NC4CCNC4)n3)cnc12] |
| 38-3 | 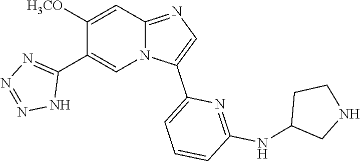 [SMILES: COc1cc2ncc(-c3cccc(NC4CCNC4)n3)n2cc1-c1nnn[nH]1] |
| 39-3 | 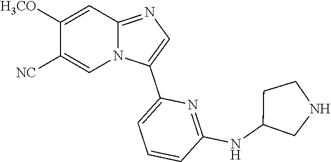 [SMILES: COc1cc2ncc(-c3cccc(NC4CCNC4)n3)n2cc1C#N] |
| 40-3 | 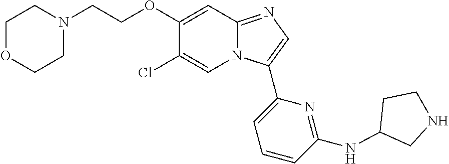 [SMILES: Clc1cn2c(-c3cccc(NC4CCNC4)n3)cnc2cc1OCCN1CCOCC1] |
| 41-3 | 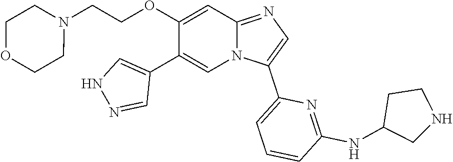 [SMILES: c1cc(NC2CCNC2)nc(-c2cnc3cc(OCCN4CCOCC4)c(-c4cn[nH]c4)cn23)c1] |
| 42-3 | 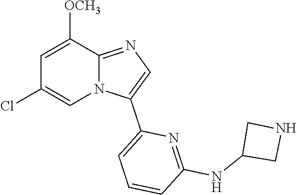 [SMILES: COc1cc(Cl)cn2c(-c3cccc(NC4CNC4)n3)cnc12] |
| 43-3 | 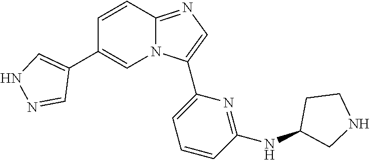 [SMILES: c1cc(N[C@H]2CCNC2)nc(-c2cnc3ccc(-c4cn[nH]c4)cn23)c1] |
| 44-3 | 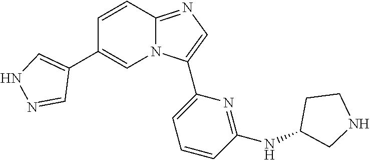 [SMILES: c1cc(N[C@@H]2CCNC2)nc(-c2cnc3ccc(-c4cn[nH]c4)cn23)c1] |
| 45-3 | 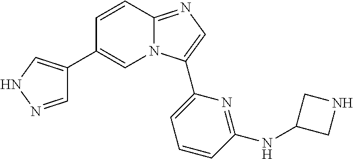 [SMILES: c1cc(NC2CNC2)nc(-c2cnc3ccc(-c4cn[nH]c4)cn23)c1] |
| 46-3 | 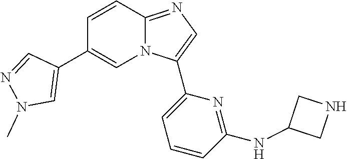 [SMILES: Cn1cc(-c2ccc3ncc(-c4cccc(NC5CNC5)n4)n3c2)cn1] |
| 47-3 | 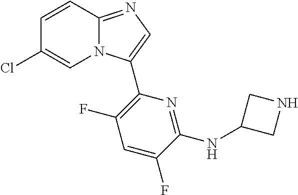 [SMILES: Fc1cc(F)c(-c2cnc3ccc(Cl)cn23)nc1NC1CNC1] |
| 48-3 | 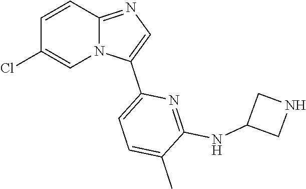 [SMILES: Cc1ccc(-c2cnc3ccc(Cl)cn23)nc1NC1CNC1] |
| 49-3 | 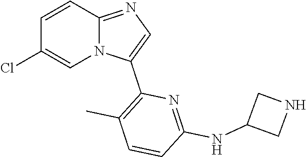 [SMILES: Cc1ccc(NC2CNC2)nc1-c1cnc2ccc(Cl)cn12] |
| 50-3 | 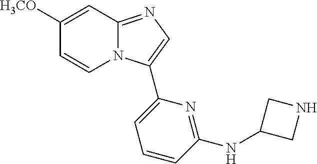 [SMILES: COc1ccn2c(-c3cccc(NC4CNC4)n3)cnc2c1] |
| 51-3 | 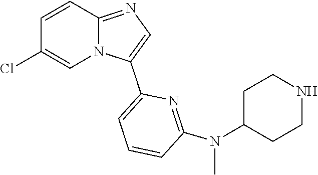 [SMILES: CN(c1cccc(-c2cnc3ccc(Cl)cn23)n1)C1CCNCC1] |
| 52-3 | 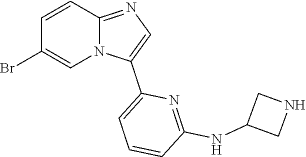 [SMILES: Brc1ccc2ncc(-c3cccc(NC4CNC4)n3)n2c1] |
| 53-3 | 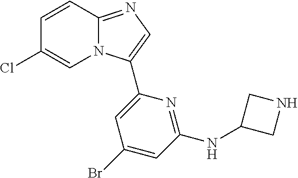 [SMILES: Clc1ccc2ncc(-c3cc(Br)cc(NC4CNC4)n3)n2c1] |
| 54-3 | 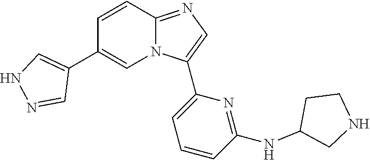 [SMILES: c1cc(NC2CCNC2)nc(-c2cnc3ccc(-c4cn[nH]c4)cn23)c1] |
| 55-3 | 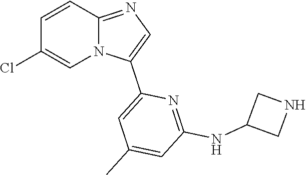 [SMILES: Cc1cc(NC2CNC2)nc(-c2cnc3ccc(Cl)cn23)c1] |
| 56-3 | 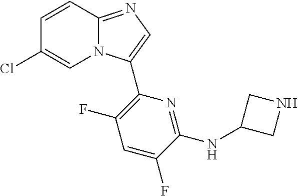 [SMILES: Fc1cc(F)c(-c2cnc3ccc(Cl)cn23)nc1NC1CNC1] |
| 57-3 | 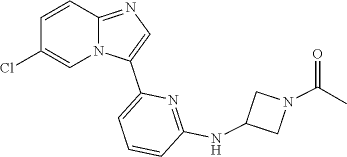 [SMILES: CC(=O)N1CC(Nc2cccc(-c3cnc4ccc(Cl)cn34)n2)C1] |
| 58-3 | 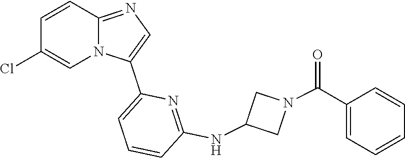 [SMILES: O=C(c1ccccc1)N1CC(Nc2cccc(-c3cnc4ccc(Cl)cn34)n2)C1] |
| 59-3 | 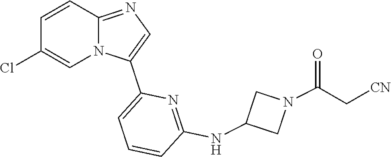 [SMILES: N#CCC(=O)N1CC(Nc2cccc(-c3cnc4ccc(Cl)cn34)n2)C1] |
| 60-3 | 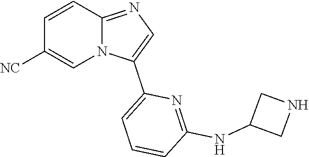 [SMILES: N#Cc1ccc2ncc(-c3cccc(NC4CNC4)n3)n2c1] |
| 61-3 | 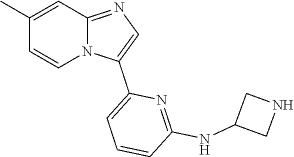 [SMILES: Cc1ccn2c(-c3cccc(NC4CNC4)n3)cnc2c1] |
| 62-3 | 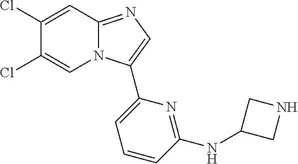 [SMILES: Clc1cc2ncc(-c3cccc(NC4CNC4)n3)n2cc1Cl] |
| 63-3 | 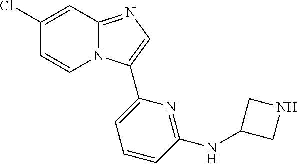 [SMILES: Clc1ccn2c(-c3cccc(NC4CNC4)n3)cnc2c1] |
| 64-3 | 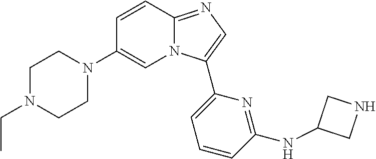 [SMILES: CCN1CCN(c2ccc3ncc(-c4cccc(NC5CNC5)n4)n3c2)CC1] |
| 65-3 | 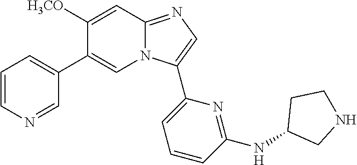 [SMILES: COc1cc2ncc(-c3cccc(N[C@@H]4CCNC4)n3)n2cc1-c1cccnc1] |
| 66-3 | 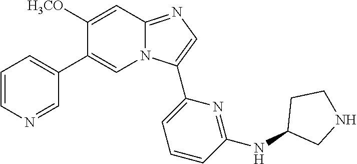 [SMILES: COc1cc2ncc(-c3cccc(N[C@H]4CCNC4)n3)n2cc1-c1cccnc1] |
| 67-3 | 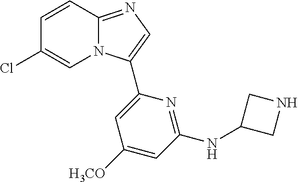 [SMILES: COc1cc(NC2CNC2)nc(-c2cnc3ccc(Cl)cn23)c1] |
| 68-3 | 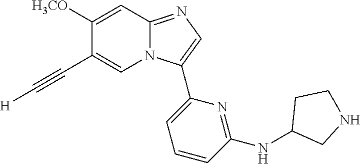 [SMILES: C#Cc1cn2c(-c3cccc(NC4CCNC4)n3)cnc2cc1OC] |

In another embodiment, the compound is a compound of Formula (VII-5008):

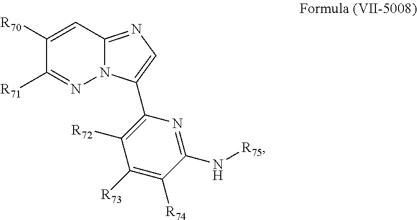

or a salt, ester, solvate, optical isomer, geometric isomer, or salt of an isomer thereof; wherein:
R₇₀ is selected from H, halogen, hydroxy, oxo, —CN, amido, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, cycloalkyl, spiro-fused cycloalkyl, heterocyclyl, aryl, heteroaryl, or fused ring heteroaryl, wherein the amido, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ heteroalkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl;
R₇₁ is selected from H, halogen, hydroxy, oxo, —CN, amino, —O-aryl, methanoyl (—COH), carboxy (—CO₂H), C₁-C₇ alkyl, C₂—C— alkenyl, C₂-C₇ alkynyl, C₁-C₇ alkoxy, cycloalkyl, heterocyclyl, spiro-fused cycloalkyl, aryl, heteroaryl, or fused ring heteroaryl, wherein the amino, —O-aryl, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, cycloalkyl, heterocyclyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ heteroalkyl, C₁-C₇ haloalkyl, C₁-C₇ perfluorinated alkyl, C₁-C₇ alkoxy, C₁-C₇ haloalkoxy, cycloalkyl, heterocyclyl, spiro-fused cycloalkyl, aryl, fused ring aryl, heteroaryl, fused ring heteroaryl, or C₁-C₇ alkyl which is substituted with cycloalkyl,
R₇₂, R₇₃, and R₇₄ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, —O-aryl, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, —O-aryl, cycloalkyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ haloalkyl, C₁-C₇ perfluorinated alkyl, C₁-C₇ alkoxy, C₁-C₇ haloalkoxy, or C₁-C₇ alkyl which is substituted with cycloalkyl;
R₇₅ is selected from

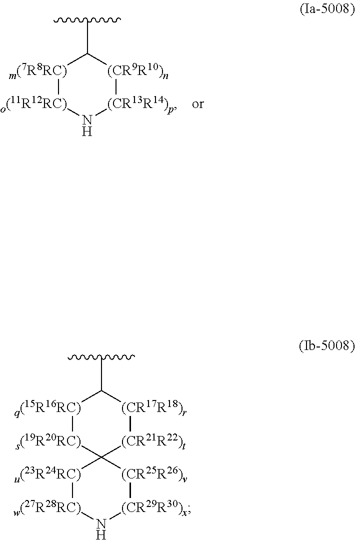

R⁷, R⁸, R⁹, R¹⁰, R¹¹, R¹², R¹³, R¹⁴ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen;
R¹⁵, R¹⁶, R¹⁷, R¹⁸, R¹⁹, R²⁰, R²¹, R²², R²³, R²⁴, R²⁵, R²⁶, R²⁷, R²⁸, R²⁹, and R³⁰ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein the methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen; and
m, n, o, p, q, r, s, t, u, v, w, and x are each independently 0, 1, 2, 3, 4, or 5; with the provisos that:
q+r+s+t is at least 1, and
u+v+w+x is at least 1.

In an embodiment, the compound of Formula (VII-5008) is a compound of Formula (IIf-5008):

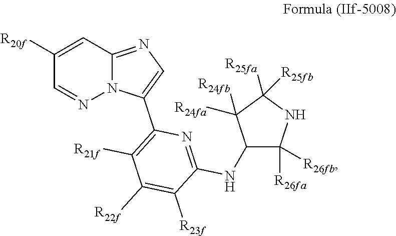

or a salt, ester, solvate, optical isomer, geometric isomer, or salt of an isomer thereof; wherein:
R_{2of} is selected from H, halogen, C₁-C₆ alkyl, C₁-C₆ alkoxy, C₃-C₆ cycloalkyl, and —O—(C₃-C₆ cycloalkyl), wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{21f}, R_{22f}, and R_{23f} are each independently selected from H and halogen; and
R_{24fa}, R_{24fh}, R_{25fa}, R_{25fb}, R_{26fa}, and R_{26fb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms.

In one embodiment, the compound of Formula (VII-5008) is a compound of Formula (IIg-5008):

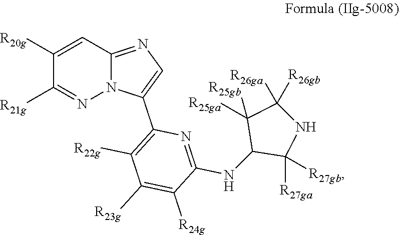

or a salt, ester, solvate, optical isomer, geometric isomer, or salt of an isomer thereof; wherein:
R_{20g} is selected from H and C₁-C₆ alkoxy;
R_{21g} is selected from halogen, C₁-C₆ alkyl, C₁-C₆ alkoxy, C₃-C₆ cycloalkyl, —O—(C₆-C₁₂ aryl), C₃-C₉ heterocyclyl, and —NR_{28ga}R_{28gb}, wherein C₁-C₆ alkyl and C₁-C₆ alkoxy are each optionally substituted with one or more substituents selected from —OH and halogen, C₃-C₆ cycloalkyl is optionally substituted with one or more substituents selected from C₁-C₆ alkyl and halogen, and C₃-C₉ heterocycyl is optionally substituted with one or more substituents selected from C₁-C₆ alkyl, C₃-C₆-cycloalkyl, C₃-C₉-heterocyclyl, —OH, and halogen;
R_{22g}, R_{23g}, and R_{24g} are each independently selected from H and halogen;
R_{25ga}, R_{25gb}, R_{26ga}, R_{26gb}, R_{27ga}, and R_{27gb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
R_{28g}a and R_{28gb} are each independently selected from H, C₁-C₆ alkyl, and C₃-C₆ cycloalkyl.

In one embodiment, the compound of Formula (VII-5008) is a compound of Formula (IIh-5008):

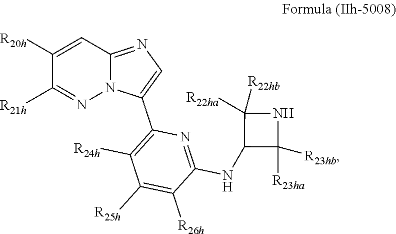

or a salt, ester, solvate, optical isomer, geometric isomer, or salt of an isomer thereof; wherein:
R₂₀ₕ is selected from H and C₁-C₆ alkoxy;
R₂₁ₕ is selected from C₁-C₆ alkyl, C₃-C₆ cycloalkyl, and C₃-C₉ heterocyclyl, wherein C₁-C₆ alkyl is optionally substituted with one or more substituents selected from —OH and halogen and C₃-C₆ cycloalkyl, and C₃-C₉ heterocyclyl are each optionally substituted with one or more substituents selected from C₁-C₆ alkyl, —OH, and halogen;
R₂₂ₕₐ, R_{221b}, R₂₃₁ₐ, and R_{231b} are each independently selected from H and C₁-C₆ alkyl, wherein C₁-C₆ alkyl is optionally substituted with one or more halogen atoms; and
R₂₄ₕ, R₂₅ₕ, and R₂₆ₕ are each independently selected from H and halogen.

In one embodiment, the compound of Formula (VII-5008) is a compound of Formula (IIi-5008):

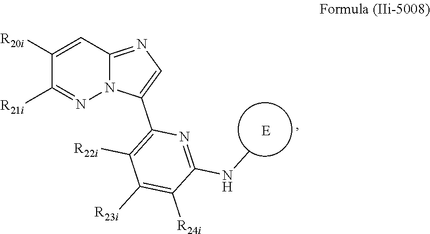

or a salt, ester, solvate, optical isomer, geometric isomer, or salt of an isomer thereof, wherein:

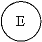

is selected from

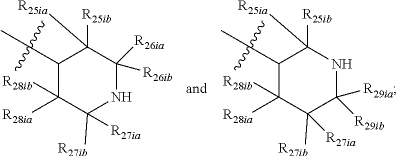

R₂₀ᵢ is selected from H, and C₁-C₆ alkoxy;
R₂ₗᵢ is selected from C₁-C₆ alkyl, C₁-C₆ alkoxy, C₃-C₆ cycloalkyl, and C₃-C₉ heterocyclyl, wherein C₁-C₆ alkyl and C₁-C₆ alkoxy are each optionally substituted with one or more substituents selected from —OH and halogen, C₃-C₆ cycloalkyl is optionally substituted with one or more substituents selected from C₁-C₆ alkyl and halogen, and C₃-C₉ heterocycyl is optionally substituted with one or more substituents selected from C₁-C₆ alkyl, C₃-C₆-cycloalkyl, C₃-C₉-heterocyclyl, —OH, —C═O, and halogen;
R₂₂ᵢ, R₂₃ᵢ, and R₂₄ᵢ are each independently selected from H and halogen; and
R₂₅ᵢₐ, R_{25ib}, R₂₆ᵢₐ, R_{26ib}, R₂₇ᵢₐ, R_{27ib}, R₂₈ᵢₐ, R_{28ib}, R₂₉ᵢₐ, and R_{29ib} are each independently selected from H, halogen, —OH, or C₁-C₆ alkyl.

In one embodiment, the compound of Formula (VII-5008) is a compound of Formula (IIj-5008):

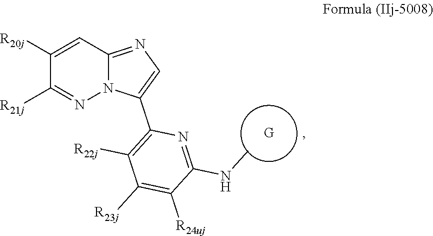

or a salt, ester, solvate, optical isomer, geometric isomer, or salt of an isomer thereof, wherein:

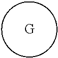

is selected from

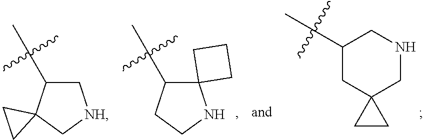 [SMILES: *C1CNCC12CC2 ; *C1CCNC12CCC2 ; *C1CNCC2(CC2)C1]

R₂₀ⱼ is selected from H, and C₁-C₆ alkoxy;
R₂₁ⱼ is selected from H, C₁-C₆ alkyl, C₁-C₆ alkoxy, and C₃-C₆ cycloalkyl, wherein C₁-C₆ alkyl and C₁-C₆ alkoxy are each optionally substituted with one or more substituents selected from halogen and —OH, and C₃-C₆ cycloalkyl is optionally substituted with one or more substituents selected from C₁-C₆ alkyl and halogen; and
R₂₂ⱼ, R₂₃ⱼ, and R₂₄ⱼ are each independently selected from H and halogen.

In one embodiment, the compound of Formula (VII-5008) is a compound of Formula (IIk-5008):

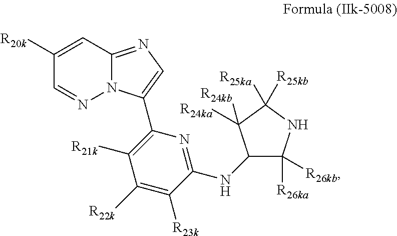

or a salt, ester, solvate, optical isomer, geometric isomer, or salt of an isomer thereof; wherein:
R₂₀ₖ is selected from H, halogen, C₁-C₆ alkyl, C₁-C₆ alkoxy, C₃-C₆ cycloalkyl, and —O—(C₃-C₆ cycloalkyl), wherein C₁-C₆ alkyl and C₁-C₆ alkoxy are each optionally substituted with one or more substituents selected from —OH and halogen, and wherein C₃-C₆ cycloalkyl and —O—(C₃-C₆ cycloalkyl) are each optionally substituted with one or more substituents selected from C₁-C₆ alkyl and halogen;
R₂₁ₖ, R₂₂ₖ, and R₂₃ₖ are each independently selected from H, halogen, C₁-C₆ alkyl, and C₁-C₆ alkoxy, wherein C₁-C₆ alkyl is optionally substituted with one or more halogen; and
R₂₄ₖₐ, R_{24kb}, R₂₅ₖₐ, R_{25kb}, R₂₆ₖₐ, and R_{26kb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms.

In one embodiment, the compound of Formula (VII-5008) is a compound of Formula (IIm-5008):

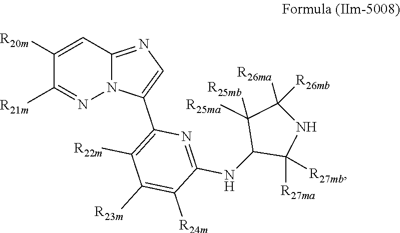

or a salt, ester, solvate, optical isomer, geometric isomer, or salt of an isomer thereof; wherein:
R₂₀ₘ is selected from C₁-C₆ alkyl and C₁-C₆ alkoxy, wherein C₁-C₆ alkyl and C₁-C₆ alkoxy are each optionally substituted with one or more substituents selected from —OH and halogen;
R₂₁ₘ is selected from halogen, C₁-C₆ alkyl, C₁-C₆ alkoxy, C₃-C₆ cycloalkyl, C₅-C₁₂ spiro-fused cycloalkyl, —O—(C₆-C₁₂ aryl), C₃-C₉ heterocyclyl, and —NR₂₈ₘₐR_{28mb}, wherein C₁-C₆ alkyl and C₁-C₆ alkoxy are each optionally substituted with one or more substituents selected from —OH and halogen, wherein C₃-C₆ cycloalkyl is optionally substituted with one or more substituents selected from C₁-C₆ alkyl and halogen, wherein C₁-C₆ alkyl is optionally substituted with one or more —OH, and wherein C₃-C₉ heterocycyl is optionally substituted with one or more substituents selected from C₁-C₆ alkyl, C₃-C₆-cycloalkyl, C₃-C₉-heterocyclyl, —OH, and halogen;
R₂₂ₘ, R₂₃ₘ, and R₂₄ₘ are each independently selected from H, halogen, C₁-C₆ alkyl, and C₁-C₆ alkoxy, wherein C₁-C₆ alkyl is optionally substituted with one or more halogen;
R₂₅ₘₐ, R_{25mb}, R₂₆ₘₐ, R_{26mb}, R₂₇ₘₐ, and R_{27mb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
R₂₈ₘₐ and R_{28mb} are each independently selected from H, C₁-C₆ alkyl, and C₃-C₆ cycloalkyl.

In one embodiment, the compound of Formula (VII-5008) is a compound of Formula (IIn-5008):

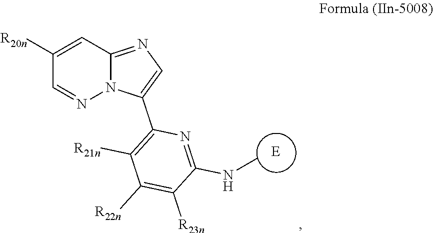

or a salt, ester, solvate, optical isomer, geometric isomer, or salt of an isomer thereof, wherein:

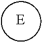

is selected from

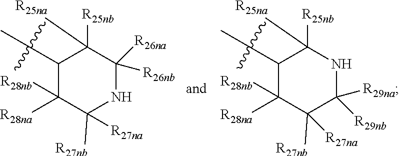

R₂₀ₙ n is selected from H, halogen, C₁-C₆ alkyl, C₁-C₆ alkoxy, C₃-C₆ cycloalkyl, and —O—(C₃-C₆ cycloalkyl), wherein C₁-C₆ alkyl and C₁-C₆ alkoxy are each optionally substituted with one or more substituents selected from —OH and halogen, and wherein C₃-C₆ cycloalkyl and —O—(C₃-C₆ cycloalkyl) are each optionally substituted with one or more substituents selected from C₁-C₆ alkyl and halogen;
R₂₁ₙ, R₂₂ₙ, and R₂₃ₙ are each independently selected from H, halogen, C₁-C₆ alkyl, and C₁-C₆ alkoxy, wherein C₁-C₆ alkyl is optionally substituted with one or more halogen; and
R₂₅ₙₐ, R_{25nb}, R₂₆ₙₐ, R_{26nb}, R₂₇ₙₐ, R_{27nb}, R₂₈ₙₐ, R_{28nb}, R₂₉ₙₐ, and R_{29nb} are each independently selected from H, halogen, —OH, C₁-C₆ alkyl, or C₁-C₆ alkoxy wherein C₁-C₆ alkyl and C₁-C₆ alkoxy are each optionally substituted with one or more halogen atoms.

In one embodiment, the compound of Formula (VII-5008) is a compound of Formula (IIp-5008):

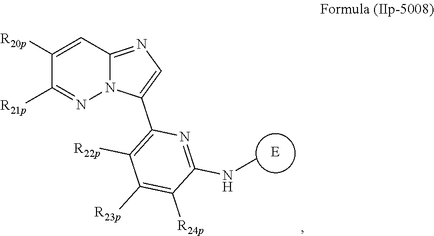

or a salt, ester, solvate, optical isomer, geometric isomer, or salt of an isomer thereof; wherein:

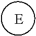

is selected from

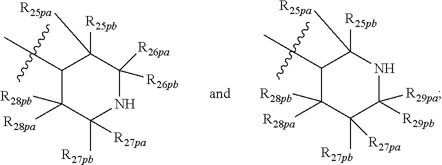

R₂₀ₚ is selected from C₁-C₆ alkyl and C₁-C₆ alkoxy, wherein C₁-C₆ alkyl and C₁-C₆ alkoxy are each optionally substituted with one or more substituents selected from —OH and halogen;
R₂₁ₚ is selected from halogen, C₁-C₆ alkyl, C₁-C₆ alkoxy, C₃-C₆ cycloalkyl, C₅-C₁₂ spiro-fused cycloalkyl, —O—(C₆-C₁₂ aryl), C₃-C₉ heterocyclyl, and —NR₂₂₀ₚₐR_{220pb}, wherein C₁-C₆ alkyl and C₁-C₆ alkoxy are each optionally substituted with one or more substituents selected from —OH and halogen, wherein C₃-C₆ cycloalkyl is optionally substituted with one or more substituents selected from C₁-C₆ alkyl and halogen, and wherein C₃-C₉ heterocycyl is optionally substituted with one or more substituents selected from C₁-C₆ alkyl, C₃-C₆-cycloalkyl, C₃-C₉-heterocyclyl, —OH, and halogen;
R₂₂ₚ, R₂₃ₚ, and R₂₄ₚ are each independently selected from H, halogen, C₁-C₆ alkyl, and C₁-C₆ alkoxy, wherein C₁-C₆ alkyl is optionally substituted with one or more halogen;
R₂₅ₚₐ, R_{25pb}, R₂₆ₚₐ, R_{26pb}, R₂₇ₚₐ, R_{27pb}, R₂₈ₚₐ, R_{28pb}, R₂₉ₚₐ, and R_{29pb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
R₂₂₀ₚₐ and R_{220pb} are each independently selected from H, C₁-C₆ alkyl, and C₃-C₆ cycloalkyl.

In one embodiment, the compound of Formula (VII-5008) is a compound of Formula (IIq-5008):

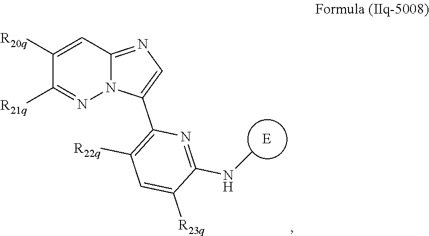

or a salt, ester, solvate, optical isomer, geometric isomer, or salt of an isomer thereof; wherein:

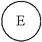

is selected from and;

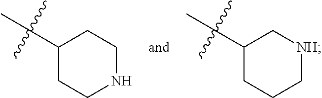 [SMILES: *C1CCNCC1 ; *C1CCCNC1]

R_{20q} is C₁-C₆ alkoxy optionally substituted with one or more substituents selected from —OH and halogen;
R_{21q} is C₃-C₆ cycloalkyl optionally substituted with one or more substituents selected from C₁-C₆ alkyl and halogen; and
R_{22q} and R_{23q} are each independently halogen.

In one embodiment, the compound of Formula (VII-5008) is a compound of Formula (IIr-5008):

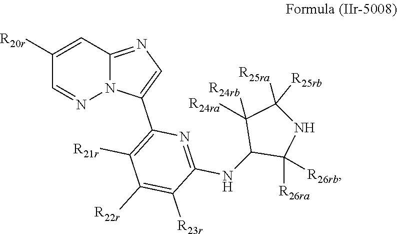

or a salt, ester, solvate, optical isomer, geometric isomer, or salt of an isomer thereof, wherein:
R₂₀ᵣ is C₁-C₆ alkoxy optionally substituted with one or more substituents selected from —OH and halogen;
R₂₁ᵣ and R₂₃ᵣ are each independently halogen;
R₂₂ᵣ is H; and
R₂₄ᵣₐ, R_{24rb}, R₂₅ᵣₐ, R_{25rb}, R₂₆ᵣₐ, and R_{26rb} are each independently selected from H and halogen, wherein one or more of R₂₄ᵣₐ, R_{24rb}, R₂₅ᵣₐ, R_{25rb}, R₂₆ᵣₐ, and R_{26rb} is halogen.

In one embodiment, the compound of Formula (VII-5008) is a compound of Formula (IIs-5008):

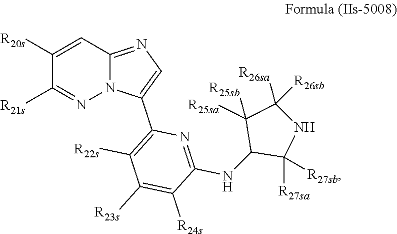

or a salt, ester, solvate, optical isomer, geometric isomer, or salt of an isomer thereof, wherein:
R₂₀ₛ is selected from C₁-C₆ alkyl, C₁-C₆ alkoxy, and —OH, wherein C₁-C₆ alkyl and C₁-C₆ alkoxy are each optionally substituted with one or more substituents selected from —OH and halogen;
R₂₁ₛ is selected from C₁-C₆ alkyl, C₃-C₆ cycloalkyl, C₅-C₁₂ spiro-fused cycloalkyl, and C₃-C₉ heterocyclyl, wherein C₁-C₆ alkyl is optionally substituted with one or more substituents selected from —OH and halogen and C₃-C₆ cycloalkyl is optionally substituted with one or more substituents selected from C₁-C₆ alkyl and halogen;
R₂₂ₛ, R₂₃ₛ, and R₂₄ₛ are each independently selected from H, CN, halogen, C₁-C₆ alkyl, C₁-C₆ alkoxy, C₃-C₆ cycloalkyl, C₆-C₁₂ aryl, and —O—(C₆-C₁₂ aryl), wherein C₁-C₆ alkyl is optionally substituted with one or more halogen; and
R₂₅ₛₐ, R_{25sb}, R₂₆ₛₐ, R_{26sb}, R₂₇ₛₐ, and R_{27sb} are each independently selected from H and halogen, wherein one or more of R₂₅ₛₐ, R_{25sb}, R₂₆ₛₐ, R_{26sb}, R₂₇ₛₐ, and R_{27sb} is halogen.

In one embodiment, the compound of Formula (VII-5008) is a compound of Formula (IIt-5008):

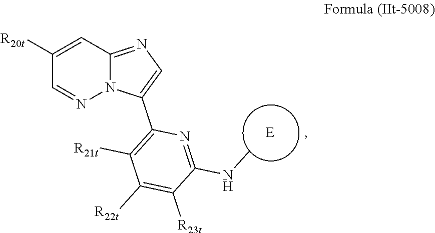

or a salt, ester, solvate, optical isomer, geometric isomer, or salt of an isomer thereof, wherein:

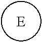

is selected from

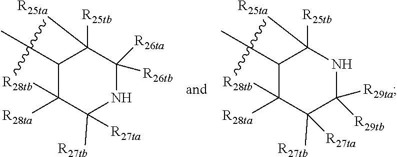

R₂₀ₜ is C₁-C₆ alkoxy optionally substituted with one or more substituents selected from —OH and halogen;
R₂₁ₜ and R₂₃ₜ are each independently halogen;
R₂₂ₜ is H; and
R₂₅ₜₐ, R_{25tb}, R₂₆ₜₐ, R_{26tb}, R₂₇ₜₐ, R_{27tb}, R₂₈ₜₐ, R_{28tb}, R₂₉ₜₐ, and R_{29tb} are each independently selected from H and halogen.

In one embodiment, the compound of Formula (VII-5008) is a compound of Formula (IIu-5008):

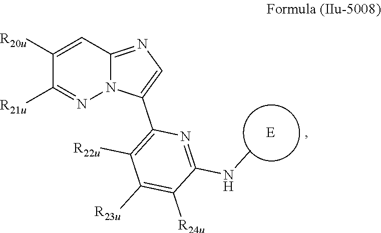

or a salt, ester, solvate, optical isomer, geometric isomer, or salt of an isomer thereof; wherein:

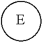

is selected from

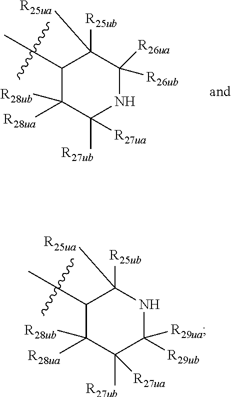

R₂₀ᵤ is selected from C₁-C₆ alkyl, C₁-C₆ alkoxy, and —OH, wherein C₁-C₆ alkyl and C₁-C₆ alkoxy are each optionally substituted with one or more substituents selected from —OH and halogen;
R₂₁ᵤ is selected from C₁-C₆ alkyl, C₃-C₆ cycloalkyl, C₅-C₁₂ spiro-fused cycloalkyl, and C₃-C₉ heterocyclyl, wherein C₁-C₆ alkyl is optionally substituted with one or more substituents selected from —OH and halogen and C₃-C₆ cycloalkyl is optionally substituted with one or more substituents selected from C₁-C₆ alkyl and halogen;
R₂₂ᵤ, R₂₃ᵤ, and R₂₄ᵤ are each independently selected from H, CN, halogen, C₁-C₆ alkyl, C₁-C₆ alkoxy, C₃-C₆ cycloalkyl, C₆-C₁₂ aryl, and —O—(C₆-C₁₂ aryl), wherein C₁-C₆ alkyl is optionally substituted with one or more halogen; and
R₂₅ᵤₐ, R_{25ub}, R₂₆ᵤₐ, R_{26ub}, R₂₇ᵤₐ, R_{27ub}, R₂₈ᵤₐ, R_{28ub}, R₂₉ᵤₐ, and R_{29ub} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms.
   In one embodiment, the compound of Formula (VII-5008) is a compound of Formula (IIIq-5008):

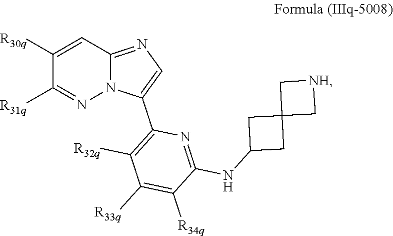

or a salt, ester, solvate, optical isomer, geometric isomer, or salt of an isomer thereof, wherein:
R_{30q} is selected from H and C₁-C₆ alkoxy;
R_{31q} is selected from C₁-C₆ alkyl, C₁-C₆ alkoxy, C₃-C₆ cycloalkyl, and C₃-C₉ heterocyclyl, wherein C₁-C₆ alkyl and C₁-C₆ alkoxy are each optionally substituted with one or more substituents selected from —OH and halogen, C₃-C₆ cycloalkyl is optionally substituted with one or more substituents selected from C₁-C₆ alkyl and halogen, and C₃-C₉ heterocycyl is optionally substituted with one or more substituents selected from C₁-C₆ alkyl, C₃-C₆-cycloalkyl, C₃-C₉-heterocyclyl, C₆-C₁₂ aryl, —OH, —C═O, and halogen; and
R_{32q}, R_{33q}, and R_{34q} are each independently selected from H and halogen.

In one embodiment, the compound of Formula (VII-5008) is a compound of Formula (IIIr-5008):

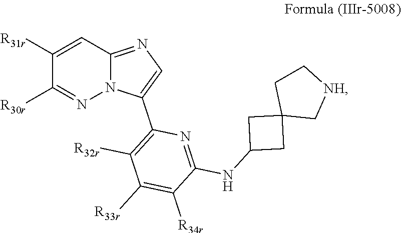

or a salt, ester, solvate, optical isomer, geometric isomer, or salt of an isomer thereof, wherein:
R₃₀ᵣ is selected from C₁-C₆ alkyl, C₁-C₆ alkoxy, C₃-C₆ cycloalkyl, and C₃-C₉ heterocyclyl, wherein C₁-C₆ alkyl and C₁-C₆ alkoxy are each optionally substituted with one or more substituents selected from —OH and halogen, C₃-C₆ cycloalkyl is optionally substituted with one or more substituents selected from C₁-C₆ alkyl and halogen, and C₃-C₉ heterocycyl is optionally substituted with one or more substituents selected from C₁-C₆ alkyl, C₃-C₆-cycloalkyl, C₃-C₉-heterocyclyl, C₆-C₁₂ aryl, —OH, —C═O, and halogen;
   R₃₁ᵣ is selected from H and C₁-C₆ alkoxy; and
   R₃₂ᵣ, R₃₃ᵣ, and R₃₄ᵣ are each independently selected from H and halogen.

In one embodiment, the compound of Formula (VII-5008) is a compound of Formula (IIIs-5008):

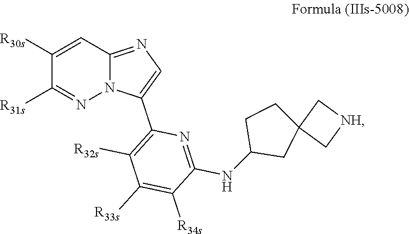

or a salt, ester, solvate, optical isomer, geometric isomer, or salt of an isomer thereof, wherein:
R₃₀ₛ is selected from H and C₁-C₆ alkoxy;
R₃₁ₛ is selected from C₁-C₆ alkyl, C₁-C₆ alkoxy, C₃-C₆ cycloalkyl, and C₃-C₉ heterocyclyl, wherein C₁-C₆ alkyl and C₁-C₆ alkoxy are each optionally substituted with one or more substituents selected from —OH and halogen, C₃-C₆ cycloalkyl is optionally substituted with one or more substituents selected from C₁-C₆ alkyl and halogen, and C₃-C₉ heterocycyl is optionally substituted with one or more substituents selected from C₁-C₆ alkyl, C₃-C₆-cycloalkyl, C₃-C₉-heterocyclyl, C₆-C₁₂ aryl, —OH, —C═O, and halogen; and
R₃₂ₛ, R₃₃ₛ, and R₃₄ₛ are each independently selected from H and halogen.

In one embodiment, the compound of Formula (VII-5008) is selected from Compounds 1-8 to 137-8 or Compounds 1a-8 to 84a-8.

| # | Structure |
|---|---|
| 1-8 | 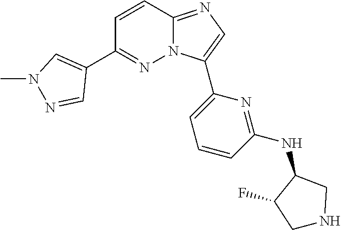 [SMILES: Cn1cc(-c2ccc3ncc(-c4cccc(N[C@H]5CNC[C@@H]5F)n4)n3n2)cn1] |
| 2-8 | 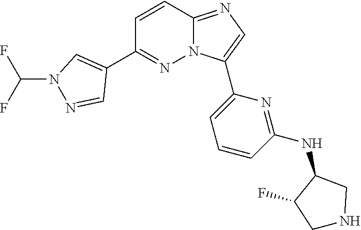 [SMILES: FC(F)n1cc(-c2ccc3ncc(-c4cccc(N[C@H]5CNC[C@@H]5F)n4)n3n2)cn1] |
| 3-8 | 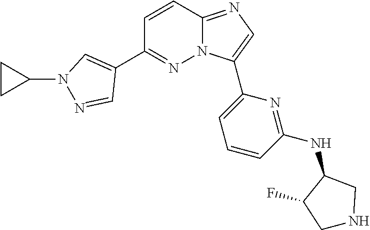 [SMILES: F[C@H]1CNC[C@@H]1Nc1cccc(-c2cnc3ccc(-c4cnn(C5CC5)c4)nn23)n1] |
| 4-8 | 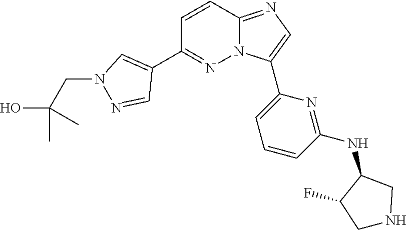 [SMILES: CC(C)(O)Cn1cc(-c2ccc3ncc(-c4cccc(N[C@H]5CNC[C@@H]5F)n4)n3n2)cn1] |
| 5-8 | 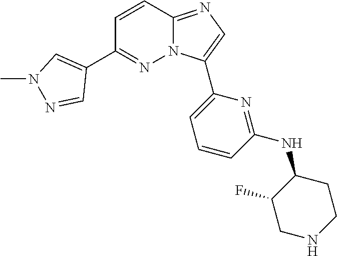 [SMILES: Cn1cc(-c2ccc3ncc(-c4cccc(N[C@H]5CCNC[C@@H]5F)n4)n3n2)cn1] |
| 6-8 | 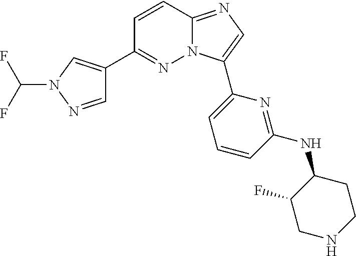 [SMILES: FC(F)n1cc(-c2ccc3ncc(-c4cccc(N[C@H]5CCNC[C@@H]5F)n4)n3n2)cn1] |
| 7-8 | 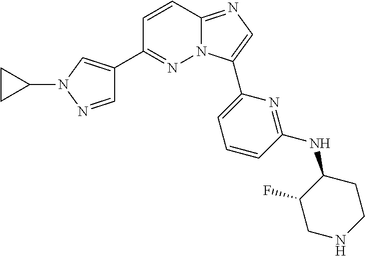 [SMILES: F[C@H]1CNCC[C@@H]1Nc1cccc(-c2cnc3ccc(-c4cnn(C5CC5)c4)nn23)n1] |
| 8-8 | 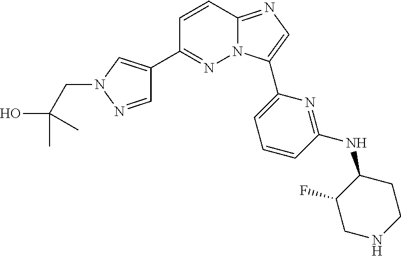 [SMILES: CC(C)(O)Cn1cc(-c2ccc3ncc(-c4cccc(N[C@H]5CCNC[C@@H]5F)n4)n3n2)cn1] |
| 9-8 | 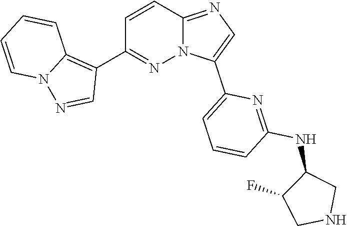 [SMILES: F[C@H]1CNC[C@@H]1Nc1cccc(-c2cnc3ccc(-c4cnn5ccccc45)nn23)n1] |
| 10-8 | 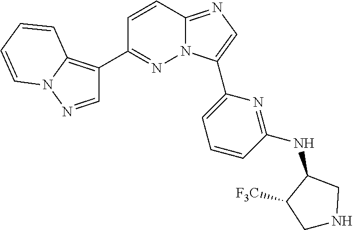 [SMILES: FC(F)(F)[C@H]1CNC[C@@H]1Nc1cccc(-c2cnc3ccc(-c4cnn5ccccc45)nn23)n1] |
| 11-8 | 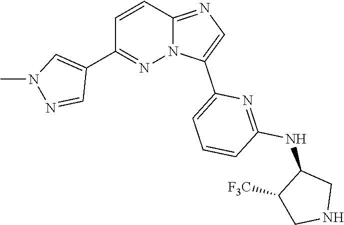 [SMILES: Cn1cc(-c2ccc3ncc(-c4cccc(N[C@H]5CNC[C@@H]5C(F)(F)F)n4)n3n2)cn1] |
| 12-8 | 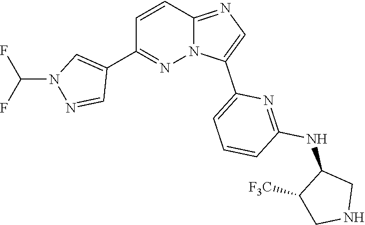 [SMILES: FC(F)n1cc(-c2ccc3ncc(-c4cccc(N[C@H]5CNC[C@@H]5C(F)(F)F)n4)n3n2)cn1] |
| 13-8 | 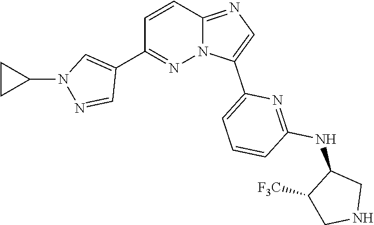 [SMILES: FC(F)(F)[C@H]1CNC[C@@H]1Nc1cccc(-c2cnc3ccc(-c4cnn(C5CC5)c4)nn23)n1] |
| 14-8 | 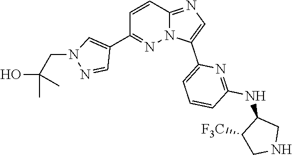 [SMILES: CC(C)(O)Cn1cc(-c2ccc3ncc(-c4cccc(N[C@H]5CNC[C@@H]5C(F)(F)F)n4)n3n2)cn1] |
| 15-8 | 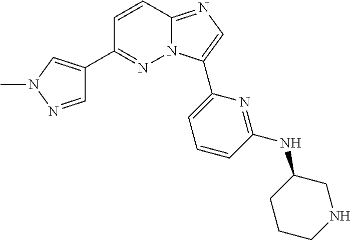 [SMILES: Cn1cc(-c2ccc3ncc(-c4cccc(N[C@@H]5CCCNC5)n4)n3n2)cn1] |
| 16-8 | 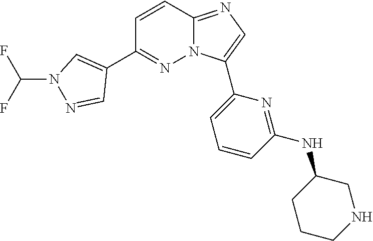 [SMILES: FC(F)n1cc(-c2ccc3ncc(-c4cccc(N[C@@H]5CCCNC5)n4)n3n2)cn1] |
| 17-8 | 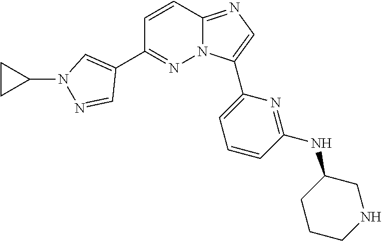 [SMILES: c1cc(N[C@@H]2CCCNC2)nc(-c2cnc3ccc(-c4cnn(C5CC5)c4)nn23)c1] |
| 18-8 | 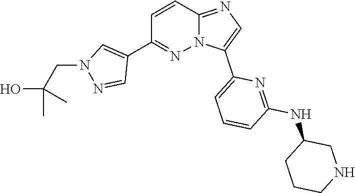 [SMILES: CC(C)(O)Cn1cc(-c2ccc3ncc(-c4cccc(N[C@@H]5CCCNC5)n4)n3n2)cn1] |
| 19-8 | 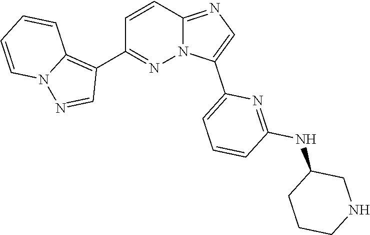 [SMILES: c1cc(N[C@@H]2CCCNC2)nc(-c2cnc3ccc(-c4cnn5ccccc45)nn23)c1] |
| 20-8 | 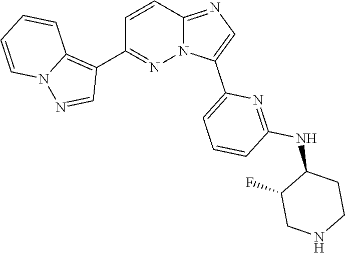 [SMILES: F[C@H]1CNCC[C@@H]1Nc1cccc(-c2cnc3ccc(-c4cnn5ccccc45)nn23)n1] |
| 21-8 | 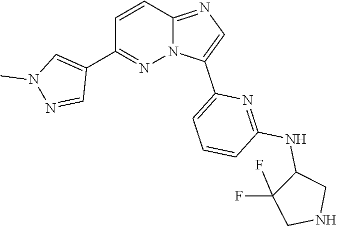 [SMILES: Cn1cc(-c2ccc3ncc(-c4cccc(NC5CNCC5(F)F)n4)n3n2)cn1] |
| 22-8 | 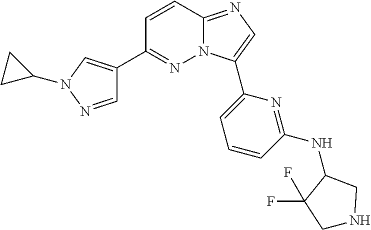 [SMILES: FC1(F)CNCC1Nc1cccc(-c2cnc3ccc(-c4cnn(C5CC5)c4)nn23)n1] |
| 23-8 | 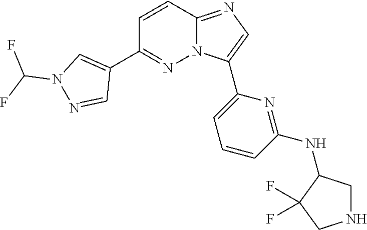 [SMILES: FC(F)n1cc(-c2ccc3ncc(-c4cccc(NC5CNCC5(F)F)n4)n3n2)cn1] |
| 24-8 | 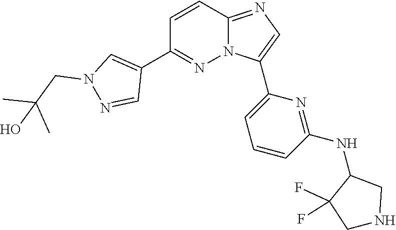 [SMILES: CC(C)(O)Cn1cc(-c2ccc3ncc(-c4cccc(NC5CNCC5(F)F)n4)n3n2)cn1] |
| 25-8 | 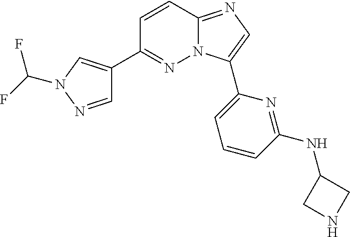 [SMILES: FC(F)n1cc(-c2ccc3ncc(-c4cccc(NC5CNC5)n4)n3n2)cn1] |
| 26-8 | 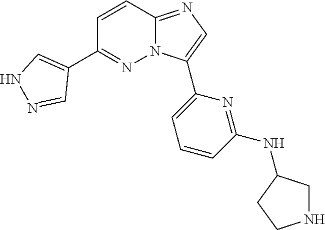 [SMILES: c1cc(NC2CCNC2)nc(-c2cnc3ccc(-c4cn[nH]c4)nn23)c1] |
| 27-8 | 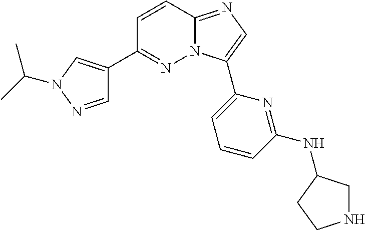 [SMILES: CC(C)n1cc(-c2ccc3ncc(-c4cccc(NC5CCNC5)n4)n3n2)cn1] |
| 28-8 | 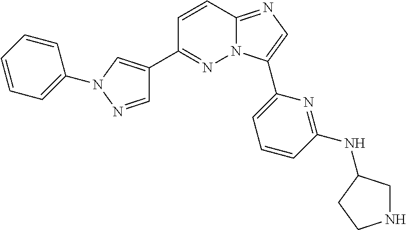 [SMILES: c1ccc(-n2cc(-c3ccc4ncc(-c5cccc(NC6CCNC6)n5)n4n3)cn2)cc1] |
| 29-8 | 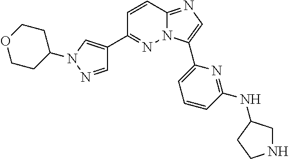 [SMILES: c1cc(NC2CCNC2)nc(-c2cnc3ccc(-c4cnn(C5CCOCC5)c4)nn23)c1] |
| 30-8 | 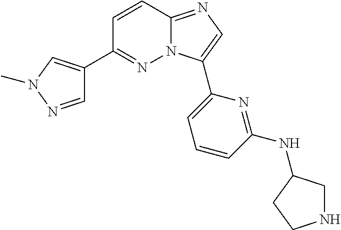 [SMILES: Cn1cc(-c2ccc3ncc(-c4cccc(NC5CCNC5)n4)n3n2)cn1] |
| 31-8 | 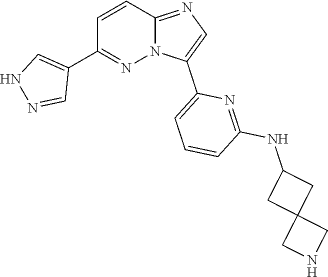 [SMILES: c1cc(NC2CC3(CNC3)C2)nc(-c2cnc3ccc(-c4cn[nH]c4)nn23)c1] |
| 32-8 | 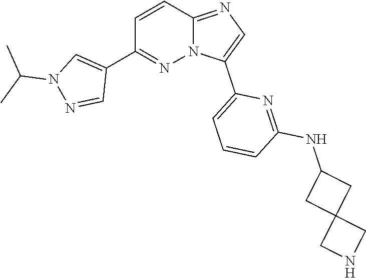 [SMILES: CC(C)n1cc(-c2ccc3ncc(-c4cccc(NC5CC6(CNC6)C5)n4)n3n2)cn1] |
| 33-8 | 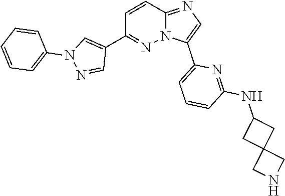 [SMILES: c1ccc(-n2cc(-c3ccc4ncc(-c5cccc(NC6CC7(CNC7)C6)n5)n4n3)cn2)cc1] |
| 34-8 | 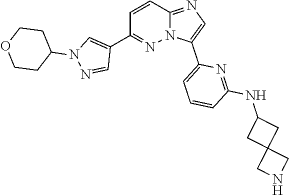 [SMILES: c1cc(NC2CC3(CNC3)C2)nc(-c2cnc3ccc(-c4cnn(C5CCOCC5)c4)nn23)c1] |
| 35-8 | 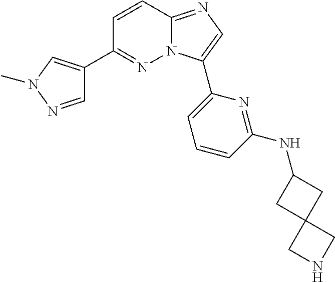 [SMILES: Cn1cc(-c2ccc3ncc(-c4cccc(NC5CC6(CNC6)C5)n4)n3n2)cn1] |
| 36-8 | 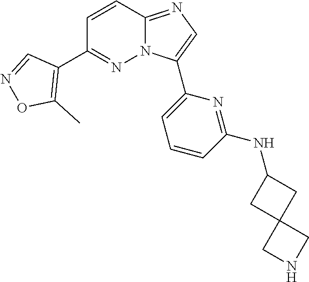 [SMILES: Cc1oncc1-c1ccc2ncc(-c3cccc(NC4CC5(CNC5)C4)n3)n2n1] |
| 37-8 | 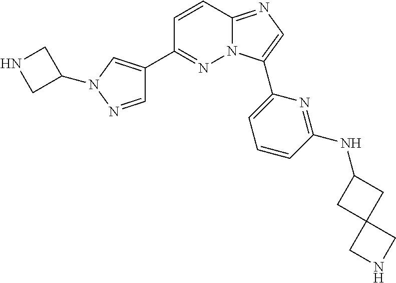 [SMILES: c1cc(NC2CC3(CNC3)C2)nc(-c2cnc3ccc(-c4cnn(C5CNC5)c4)nn23)c1] |
| 38-8 | 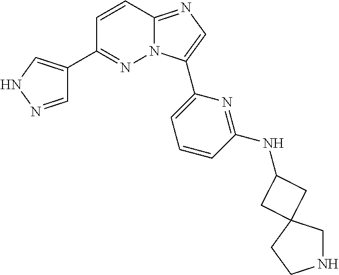 [SMILES: c1cc(NC2CC3(CCNC3)C2)nc(-c2cnc3ccc(-c4cn[nH]c4)nn23)c1] |
| 39-8 | 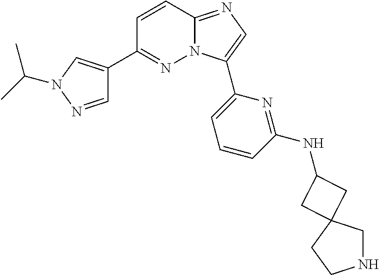 [SMILES: CC(C)n1cc(-c2ccc3ncc(-c4cccc(NC5CC6(CCNC6)C5)n4)n3n2)cn1] |
| 40-8 | 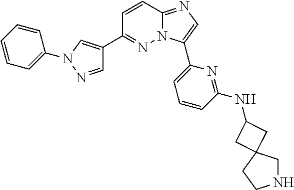 [SMILES: c1ccc(-n2cc(-c3ccc4ncc(-c5cccc(NC6CC7(CCNC7)C6)n5)n4n3)cn2)cc1] |
| 41-8 | 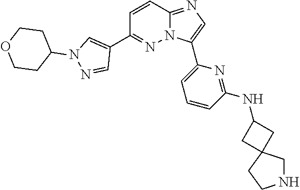 [SMILES: c1cc(NC2CC3(CCNC3)C2)nc(-c2cnc3ccc(-c4cnn(C5CCOCC5)c4)nn23)c1] |
| 42-8 | 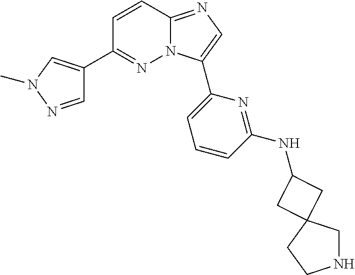 [SMILES: Cn1cc(-c2ccc3ncc(-c4cccc(NC5CC6(CCNC6)C5)n4)n3n2)cn1] |
| 43-8 | 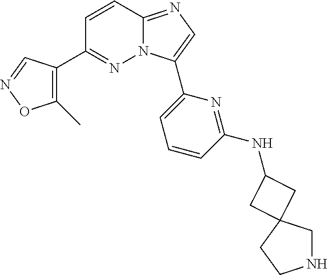 [SMILES: Cc1oncc1-c1ccc2ncc(-c3cccc(NC4CC5(CCNC5)C4)n3)n2n1] |
| 44-8 | 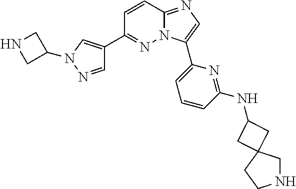 [SMILES: c1cc(NC2CC3(CCNC3)C2)nc(-c2cnc3ccc(-c4cnn(C5CNC5)c4)nn23)c1] |
| 45-8 | 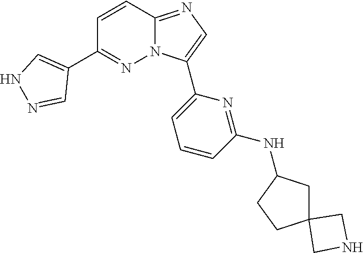 [SMILES: c1cc(NC2CCC3(CNC3)C2)nc(-c2cnc3ccc(-c4cn[nH]c4)nn23)c1] |
| 46-8 | 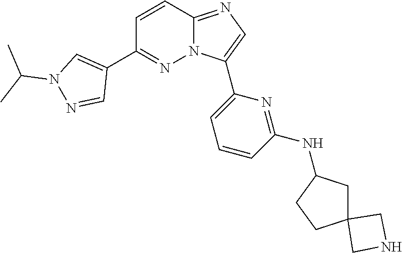 [SMILES: CC(C)n1cc(-c2ccc3ncc(-c4cccc(NC5CCC6(CNC6)C5)n4)n3n2)cn1] |
| 47-8 | 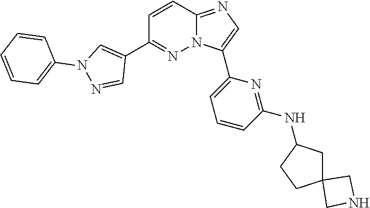 [SMILES: c1ccc(-n2cc(-c3ccc4ncc(-c5cccc(NC6CCC7(CNC7)C6)n5)n4n3)cn2)cc1] |
| 48-8 | 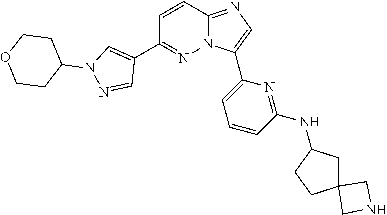 [SMILES: c1cc(NC2CCC3(CNC3)C2)nc(-c2cnc3ccc(-c4cnn(C5CCOCC5)c4)nn23)c1] |
| 49-8 | 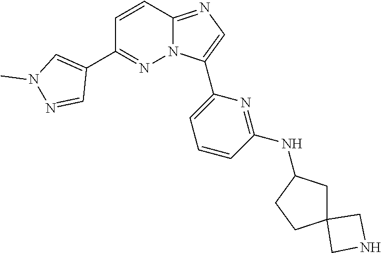 [SMILES: Cn1cc(-c2ccc3ncc(-c4cccc(NC5CCC6(CNC6)C5)n4)n3n2)cn1] |
| 50-8 | 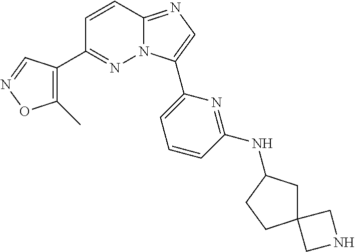 [SMILES: Cc1oncc1-c1ccc2ncc(-c3cccc(NC4CCC5(CNC5)C4)n3)n2n1] |
| 51-8 | 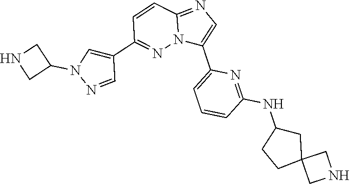 [SMILES: c1cc(NC2CCC3(CNC3)C2)nc(-c2cnc3ccc(-c4cnn(C5CNC5)c4)nn23)c1] |
| 52-8 | 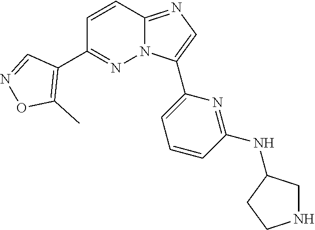 [SMILES: Cc1oncc1-c1ccc2ncc(-c3cccc(NC4CCNC4)n3)n2n1] |
| 53-8 | 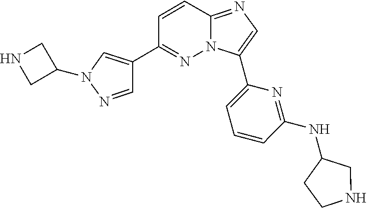 [SMILES: c1cc(NC2CCNC2)nc(-c2cnc3ccc(-c4cnn(C5CNC5)c4)nn23)c1] |
| 54-8 | 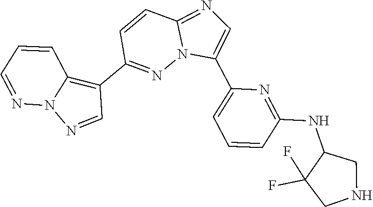 [SMILES: FC1(F)CNCC1Nc1cccc(-c2cnc3ccc(-c4cnn5ncccc45)nn23)n1] |
| 55-8 | 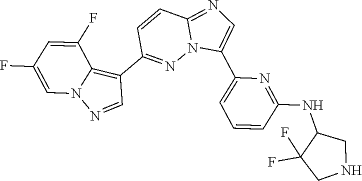 [SMILES: Fc1cc(F)c2c(-c3ccc4ncc(-c5cccc(NC6CNCC6(F)F)n5)n4n3)cnn2c1] |
| 56-8 | 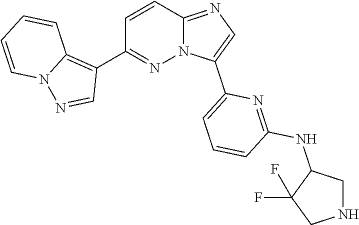 [SMILES: FC1(F)CNCC1Nc1cccc(-c2cnc3ccc(-c4cnn5ccccc45)nn23)n1] |
| 57-8 | 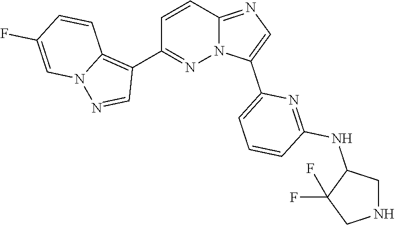 [SMILES: Fc1ccc2c(-c3ccc4ncc(-c5cccc(NC6CNCC6(F)F)n5)n4n3)cnn2c1] |
| 58-8 | 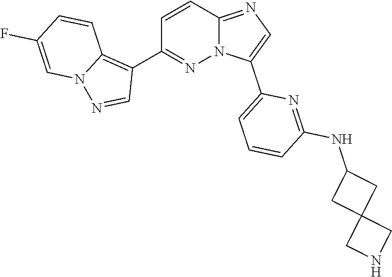 [SMILES: Fc1ccc2c(-c3ccc4ncc(-c5cccc(NC6CC7(CNC7)C6)n5)n4n3)cnn2c1] |
| 59-8 | 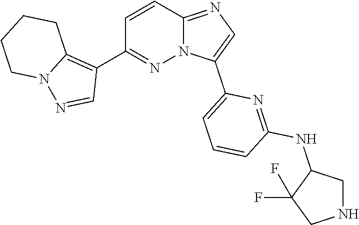 [SMILES: FC1(F)CNCC1Nc1cccc(-c2cnc3ccc(-c4cnn5c4CCCC5)nn23)n1] |
| 60-8 | 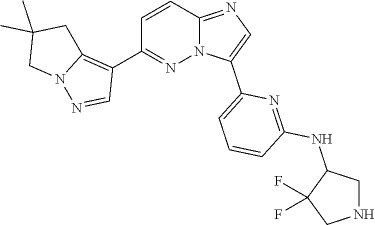 [SMILES: CC1(C)Cc2c(-c3ccc4ncc(-c5cccc(NC6CNCC6(F)F)n5)n4n3)cnn2C1] |
| 61-8 | 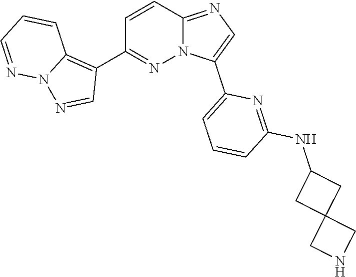 [SMILES: c1cc(NC2CC3(CNC3)C2)nc(-c2cnc3ccc(-c4cnn5ncccc45)nn23)c1] |
| 62-8 | 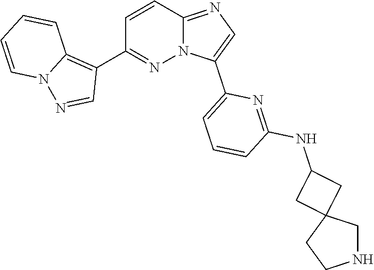 [SMILES: c1cc(NC2CC3(CCNC3)C2)nc(-c2cnc3ccc(-c4cnn5ccccc45)nn23)c1] |
| 63-8 | 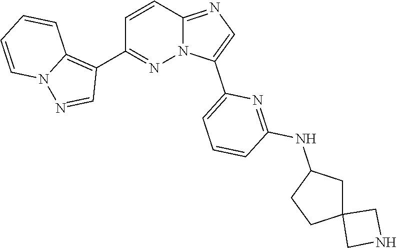 [SMILES: c1cc(NC2CCC3(CNC3)C2)nc(-c2cnc3ccc(-c4cnn5ccccc45)nn23)c1] |
| 64-8 | 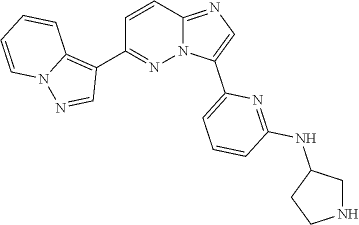 [SMILES: c1cc(NC2CCNC2)nc(-c2cnc3ccc(-c4cnn5ccccc45)nn23)c1] |
| 65-8 | 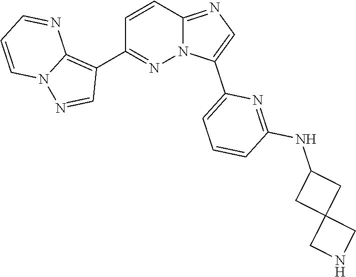 [SMILES: c1cc(NC2CC3(CNC3)C2)nc(-c2cnc3ccc(-c4cnn5cccnc45)nn23)c1] |
| 66-8 | 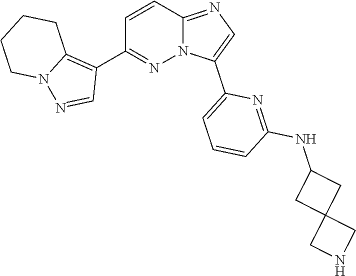 [SMILES: c1cc(NC2CC3(CNC3)C2)nc(-c2cnc3ccc(-c4cnn5c4CCCC5)nn23)c1] |
| 67-8 | 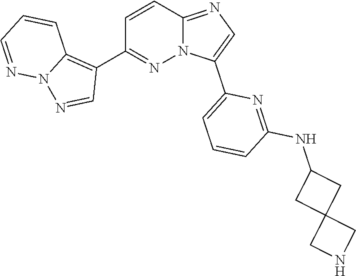 [SMILES: c1cc(NC2CC3(CNC3)C2)nc(-c2cnc3ccc(-c4cnn5ncccc45)nn23)c1] |
| 68-8 | 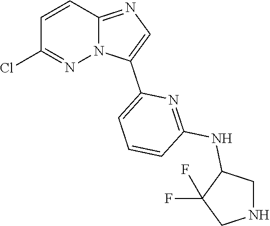 [SMILES: FC1(F)CNCC1Nc1cccc(-c2cnc3ccc(Cl)nn23)n1] |
| 69-8 | 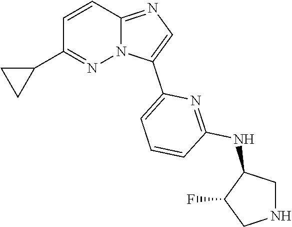 [SMILES: F[C@H]1CNC[C@@H]1Nc1cccc(-c2cnc3ccc(C4CC4)nn23)n1] |
| 70-8 | 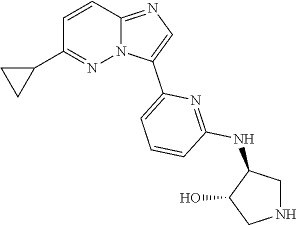 [SMILES: O[C@H]1CNC[C@@H]1Nc1cccc(-c2cnc3ccc(C4CC4)nn23)n1] |
| 71-8 | 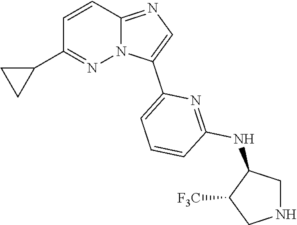 [SMILES: FC(F)(F)[C@H]1CNC[C@@H]1Nc1cccc(-c2cnc3ccc(C4CC4)nn23)n1] |
| 72-8 | 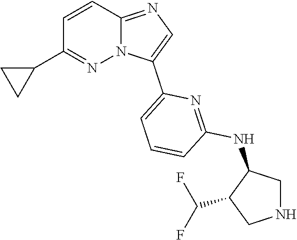 [SMILES: FC(F)[C@H]1CNC[C@@H]1Nc1cccc(-c2cnc3ccc(C4CC4)nn23)n1] |
| 73-8 | 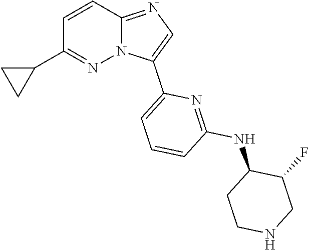 [SMILES: F[C@@H]1CNCC[C@H]1Nc1cccc(-c2cnc3ccc(C4CC4)nn23)n1] |
| 74-8 | 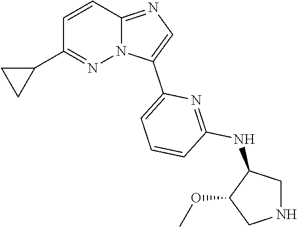 [SMILES: CO[C@H]1CNC[C@@H]1Nc1cccc(-c2cnc3ccc(C4CC4)nn23)n1] |
| 75-8 | 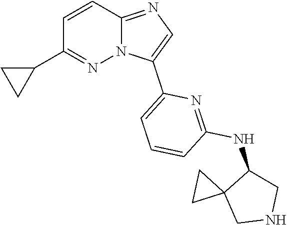 [SMILES: c1cc(N[C@H]2CNCC23CC3)nc(-c2cnc3ccc(C4CC4)nn23)c1] |
| 76-8 | 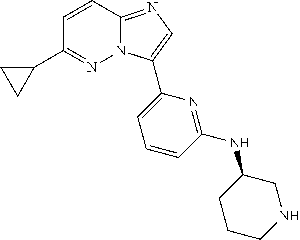 [SMILES: c1cc(N[C@@H]2CCCNC2)nc(-c2cnc3ccc(C4CC4)nn23)c1] |
| 77-8 | 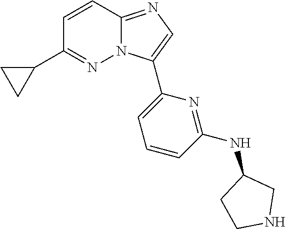 [SMILES: c1cc(N[C@@H]2CCNC2)nc(-c2cnc3ccc(C4CC4)nn23)c1] |
| 78-8 | 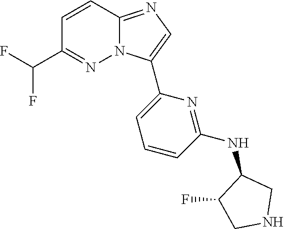 [SMILES: FC(F)c1ccc2ncc(-c3cccc(N[C@H]4CNC[C@@H]4F)n3)n2n1] |
| 79-8 | 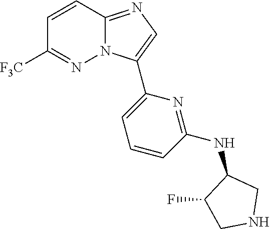 [SMILES: F[C@H]1CNC[C@@H]1Nc1cccc(-c2cnc3ccc(C(F)(F)F)nn23)n1] |
| 80-8 | 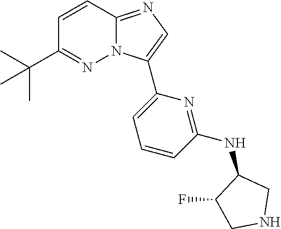 [SMILES: CC(C)(C)c1ccc2ncc(-c3cccc(N[C@H]4CNC[C@@H]4F)n3)n2n1] |
| 81-8 | 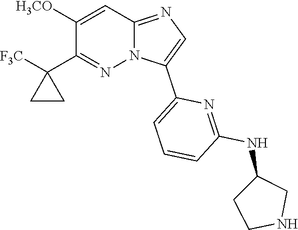 [SMILES: COc1cc2ncc(-c3cccc(N[C@@H]4CCNC4)n3)n2nc1C1(C(F)(F)F)CC1] |
| 82-8 | 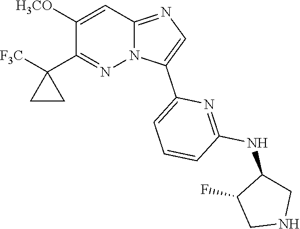 [SMILES: COc1cc2ncc(-c3cccc(N[C@H]4CNC[C@@H]4F)n3)n2nc1C1(C(F)(F)F)CC1] |
| 83-8 | 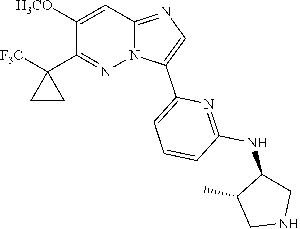 [SMILES: COc1cc2ncc(-c3cccc(N[C@H]4CNC[C@@H]4C)n3)n2nc1C1(C(F)(F)F)CC1] |
| 84-8 | 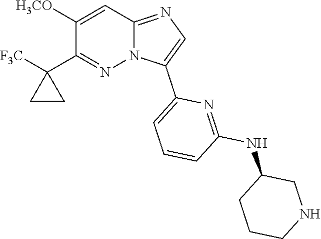 [SMILES: COc1cc2ncc(-c3cccc(N[C@@H]4CCCNC4)n3)n2nc1C1(C(F)(F)F)CC1] |
| 85-8 | 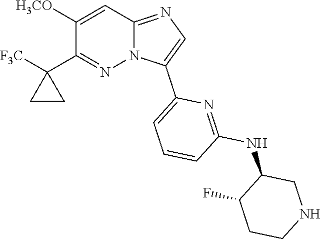 [SMILES: COc1cc2ncc(-c3cccc(N[C@H]4CNCC[C@@H]4F)n3)n2nc1C1(C(F)(F)F)CC1] |
| 86-8 | 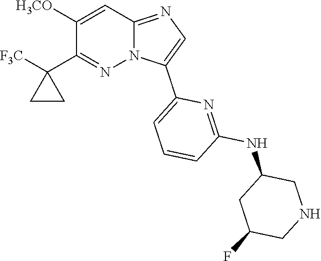 [SMILES: COc1cc2ncc(-c3cccc(N[C@H]4CNC[C@@H](F)C4)n3)n2nc1C1(C(F)(F)F)CC1] |
| 87-8 | 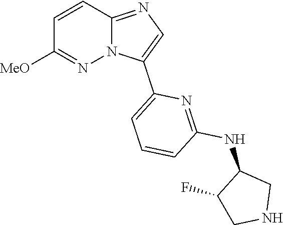 [SMILES: COc1ccc2ncc(-c3cccc(N[C@H]4CNC[C@@H]4F)n3)n2n1] |
| 88-8 | 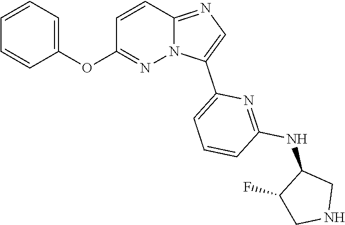 [SMILES: F[C@H]1CNC[C@@H]1Nc1cccc(-c2cnc3ccc(Oc4ccccc4)nn23)n1] |
| 89-8 | 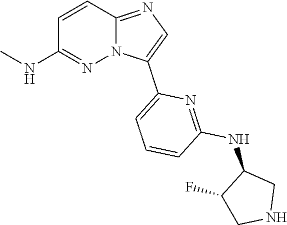 [SMILES: CNc1ccc2ncc(-c3cccc(N[C@H]4CNC[C@@H]4F)n3)n2n1] |
| 90-8 | 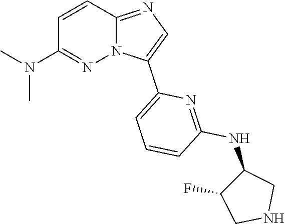 [SMILES: CN(C)c1ccc2ncc(-c3cccc(N[C@H]4CNC[C@@H]4F)n3)n2n1] |
| 91-8 | 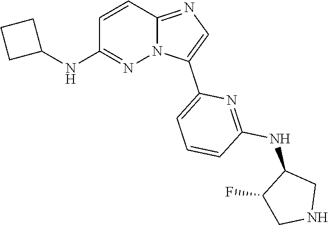 [SMILES: F[C@H]1CNC[C@@H]1Nc1cccc(-c2cnc3ccc(NC4CCC4)nn23)n1] |
| 92-8 | 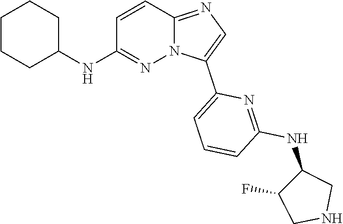 [SMILES: F[C@H]1CNC[C@@H]1Nc1cccc(-c2cnc3ccc(NC4CCCCC4)nn23)n1] |
| 93-8 | 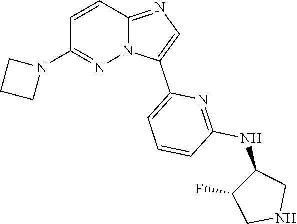 [SMILES: F[C@H]1CNC[C@@H]1Nc1cccc(-c2cnc3ccc(N4CCC4)nn23)n1] |
| 94-8 | 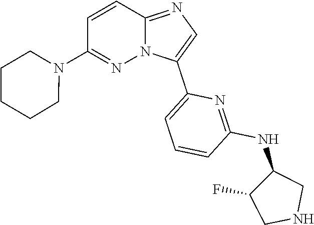 [SMILES: F[C@H]1CNC[C@@H]1Nc1cccc(-c2cnc3ccc(N4CCCCC4)nn23)n1] |
| 95-8 | 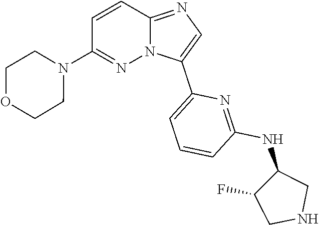 [SMILES: F[C@H]1CNC[C@@H]1Nc1cccc(-c2cnc3ccc(N4CCOCC4)nn23)n1] |
| 96-8 | 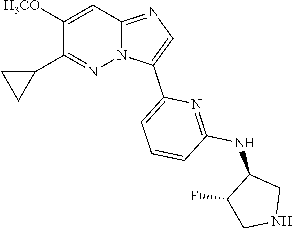 [SMILES: COc1cc2ncc(-c3cccc(N[C@H]4CNC[C@@H]4F)n3)n2nc1C1CC1] |
| 97-8 | 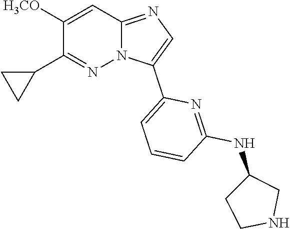 [SMILES: COc1cc2ncc(-c3cccc(N[C@@H]4CCNC4)n3)n2nc1C1CC1] |
| 98-8 | 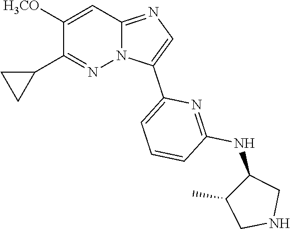 [SMILES: COc1cc2ncc(-c3cccc(N[C@H]4CNC[C@@H]4C)n3)n2nc1C1CC1] |
| 99-8 | 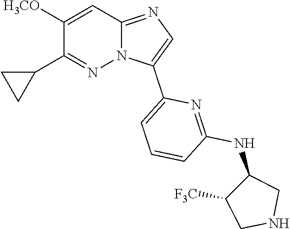 [SMILES: COc1cc2ncc(-c3cccc(N[C@H]4CNC[C@@H]4C(F)(F)F)n3)n2nc1C1CC1] |
| 100-8 | 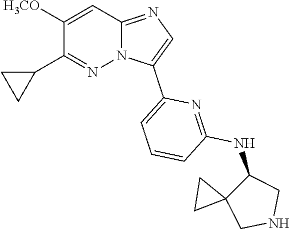 [SMILES: COc1cc2ncc(-c3cccc(N[C@H]4CNCC45CC5)n3)n2nc1C1CC1] |
| 101-8 | 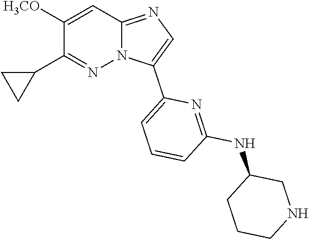 [SMILES: COc1cc2ncc(-c3cccc(N[C@@H]4CCCNC4)n3)n2nc1C1CC1] |
| 102-8 | 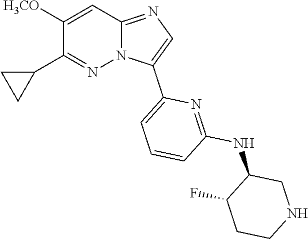 [SMILES: COc1cc2ncc(-c3cccc(N[C@H]4CNCC[C@@H]4F)n3)n2nc1C1CC1] |
| 103-8 | 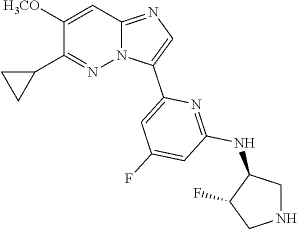 [SMILES: COc1cc2ncc(-c3cc(F)cc(N[C@H]4CNC[C@@H]4F)n3)n2nc1C1CC1] |
| 104-8 | 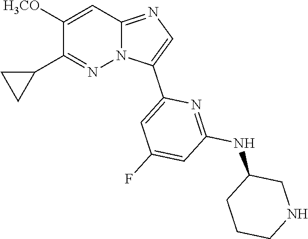 [SMILES: COc1cc2ncc(-c3cc(F)cc(N[C@@H]4CCCNC4)n3)n2nc1C1CC1] |
| 105-8 | 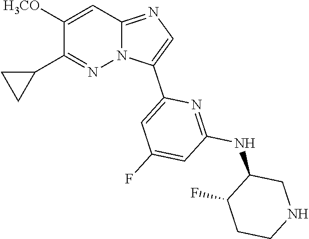 [SMILES: COc1cc2ncc(-c3cc(F)cc(N[C@H]4CNCC[C@@H]4F)n3)n2nc1C1CC1] |
| 106-8 | 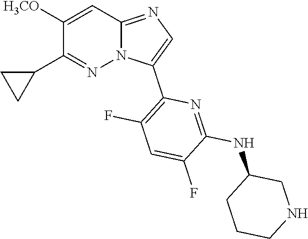 [SMILES: COc1cc2ncc(-c3nc(N[C@@H]4CCCNC4)c(F)cc3F)n2nc1C1CC1] |
| 107-8 | 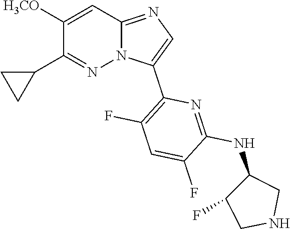 [SMILES: COc1cc2ncc(-c3nc(N[C@H]4CNC[C@@H]4F)c(F)cc3F)n2nc1C1CC1] |
| 108-8 | 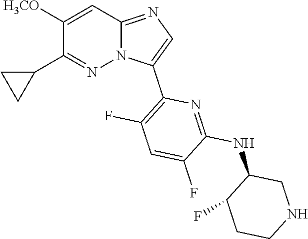 [SMILES: COc1cc2ncc(-c3nc(N[C@H]4CNCC[C@@H]4F)c(F)cc3F)n2nc1C1CC1] |
| 109-8 | 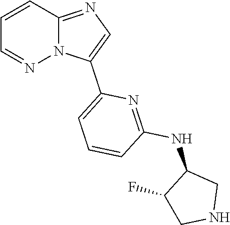 [SMILES: F[C@H]1CNC[C@@H]1Nc1cccc(-c2cnc3cccnn23)n1] |
| 110-8 | 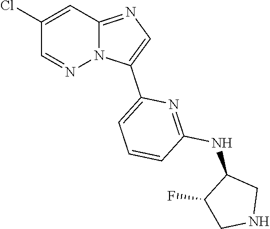 [SMILES: F[C@H]1CNC[C@@H]1Nc1cccc(-c2cnc3cc(Cl)cnn23)n1] |
| 111-8 | 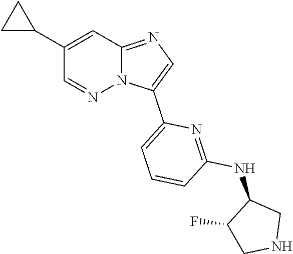 [SMILES: F[C@H]1CNC[C@@H]1Nc1cccc(-c2cnc3cc(C4CC4)cnn23)n1] |
| 112-8 | 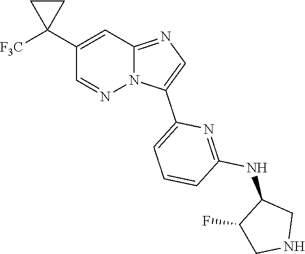 [SMILES: F[C@H]1CNC[C@@H]1Nc1cccc(-c2cnc3cc(C4(C(F)(F)F)CC4)cnn23)n1] |
| 113-8 | 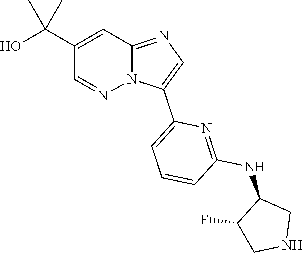 [SMILES: CC(C)(O)c1cnn2c(-c3cccc(N[C@H]4CNC[C@@H]4F)n3)cnc2c1] |
| 114-8 | 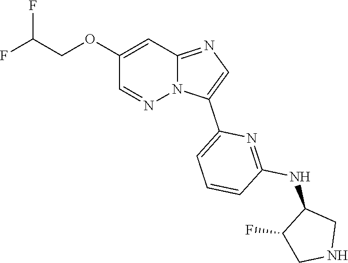 [SMILES: FC(F)COc1cnn2c(-c3cccc(N[C@H]4CNC[C@@H]4F)n3)cnc2c1] |
| 115-8 | 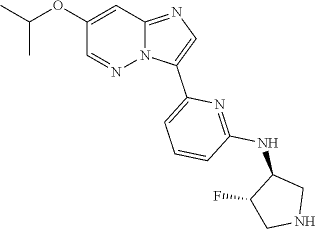 [SMILES: CC(C)Oc1cnn2c(-c3cccc(N[C@H]4CNC[C@@H]4F)n3)cnc2c1] |
| 116-8 | 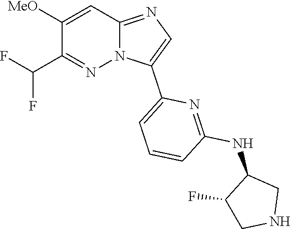 [SMILES: COc1cc2ncc(-c3cccc(N[C@H]4CNC[C@@H]4F)n3)n2nc1C(F)F] |
| 117-8 | 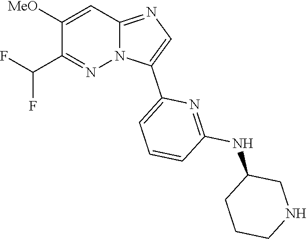 [SMILES: COc1cc2ncc(-c3cccc(N[C@@H]4CCCNC4)n3)n2nc1C(F)F] |
| 118-8 | 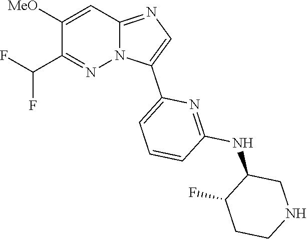 [SMILES: COc1cc2ncc(-c3cccc(N[C@H]4CNCC[C@@H]4F)n3)n2nc1C(F)F] |
| 119-8 | 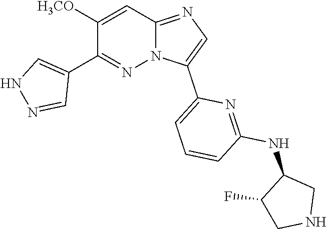 [SMILES: COc1cc2ncc(-c3cccc(N[C@H]4CNC[C@@H]4F)n3)n2nc1-c1cn[nH]c1] |
| 120-8 | 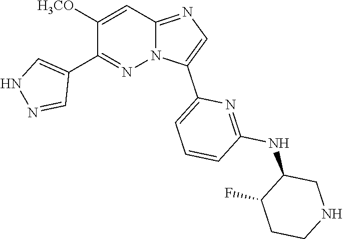 [SMILES: COc1cc2ncc(-c3cccc(N[C@H]4CNCC[C@@H]4F)n3)n2nc1-c1cn[nH]c1] |
| 121-8 | 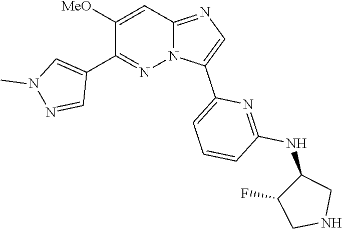 [SMILES: COc1cc2ncc(-c3cccc(N[C@H]4CNC[C@@H]4F)n3)n2nc1-c1cnn(C)c1] |
| 122-8 | 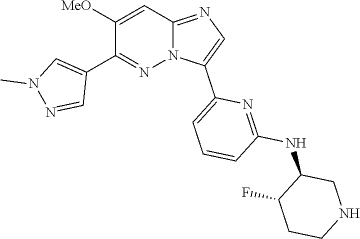 [SMILES: COc1cc2ncc(-c3cccc(N[C@H]4CNCC[C@@H]4F)n3)n2nc1-c1cnn(C)c1] |
| 123-8 | 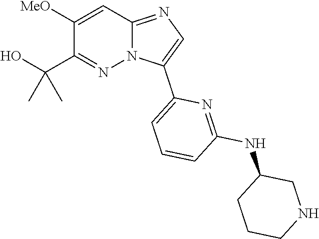 [SMILES: COc1cc2ncc(-c3cccc(N[C@@H]4CCCNC4)n3)n2nc1C(C)(C)O] |
| 124-8 | 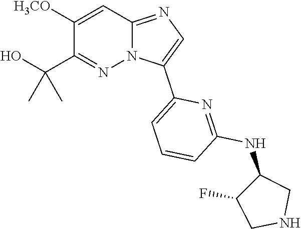 [SMILES: COc1cc2ncc(-c3cccc(N[C@H]4CNC[C@@H]4F)n3)n2nc1C(C)(C)O] |
| 125-8 | 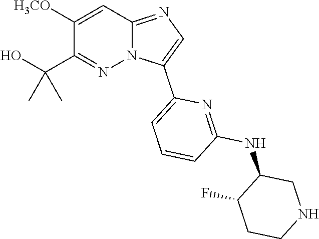 [SMILES: COc1cc2ncc(-c3cccc(N[C@H]4CNCC[C@@H]4F)n3)n2nc1C(C)(C)O] |
| 126-8 | 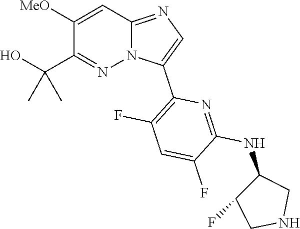 [SMILES: COc1cc2ncc(-c3nc(N[C@H]4CNC[C@@H]4F)c(F)cc3F)n2nc1C(C)(C)O] |
| 127-8 | 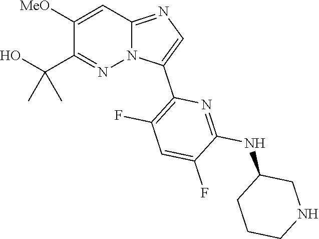 [SMILES: COc1cc2ncc(-c3nc(N[C@@H]4CCCNC4)c(F)cc3F)n2nc1C(C)(C)O] |
| 128-8 | 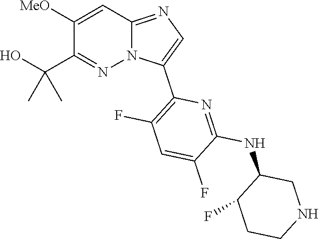 [SMILES: COc1cc2ncc(-c3nc(N[C@H]4CNCC[C@@H]4F)c(F)cc3F)n2nc1C(C)(C)O] |
| 129-8 | 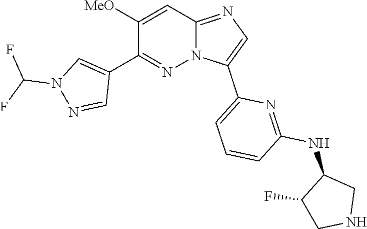 [SMILES: COc1cc2ncc(-c3cccc(N[C@H]4CNC[C@@H]4F)n3)n2nc1-c1cnn(C(F)F)c1] |
| 130-8 | 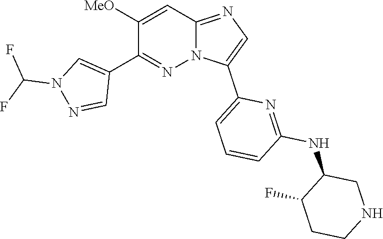 [SMILES: COc1cc2ncc(-c3cccc(N[C@H]4CNCC[C@@H]4F)n3)n2nc1-c1cnn(C(F)F)c1] |
| 131-8 | 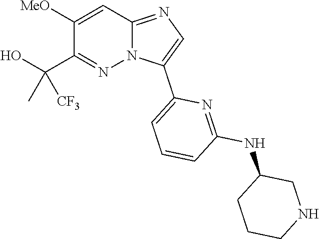 [SMILES: COc1cc2ncc(-c3cccc(N[C@@H]4CCCNC4)n3)n2nc1C(C)(O)C(F)(F)F] |
| 132-8 | 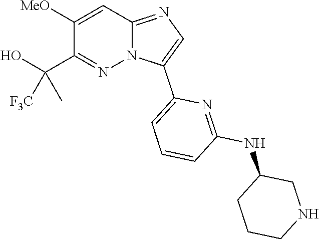 [SMILES: COc1cc2ncc(-c3cccc(N[C@@H]4CCCNC4)n3)n2nc1C(C)(O)C(F)(F)F] |
| 133-8 | 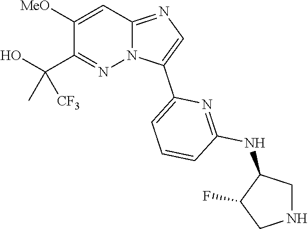 [SMILES: COc1cc2ncc(-c3cccc(N[C@H]4CNC[C@@H]4F)n3)n2nc1C(C)(O)C(F)(F)F] |
| 134-8 | 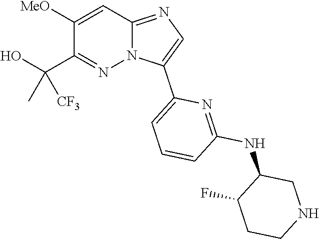 [SMILES: COc1cc2ncc(-c3cccc(N[C@H]4CNCC[C@@H]4F)n3)n2nc1C(C)(O)C(F)(F)F] |
| 135-8 | 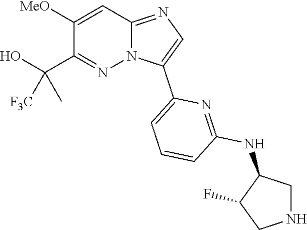 [SMILES: COc1cc2ncc(-c3cccc(N[C@H]4CNC[C@@H]4F)n3)n2nc1C(C)(O)C(F)(F)F] |
| 136-8 | 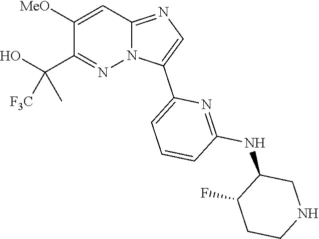 [SMILES: COc1cc2ncc(-c3cccc(N[C@H]4CNCC[C@@H]4F)n3)n2nc1C(C)(O)C(F)(F)F] |
| 137-8 | 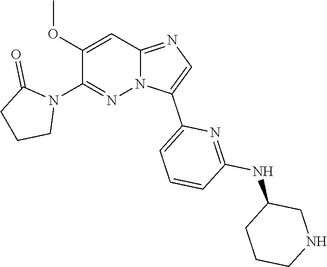 [SMILES: COc1cc2ncc(-c3cccc(N[C@@H]4CCCNC4)n3)n2nc1N1CCCC1=O] |
| 1a-8 | 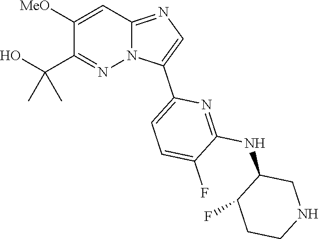 [SMILES: COc1cc2ncc(-c3ccc(F)c(N[C@H]4CNCC[C@@H]4F)n3)n2nc1C(C)(C)O] |
| 2a-8 | 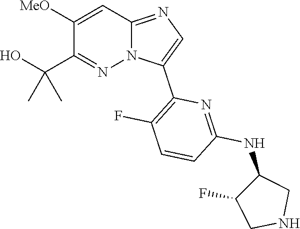 [SMILES: COc1cc2ncc(-c3nc(N[C@H]4CNC[C@@H]4F)ccc3F)n2nc1C(C)(C)O] |
| 3a-8 | 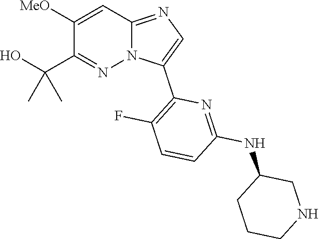 [SMILES: COc1cc2ncc(-c3nc(N[C@@H]4CCCNC4)ccc3F)n2nc1C(C)(C)O] |
| 4a-8 | 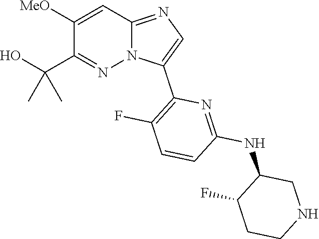 [SMILES: COc1cc2ncc(-c3nc(N[C@H]4CNCC[C@@H]4F)ccc3F)n2nc1C(C)(C)O] |
| 5a-8 | 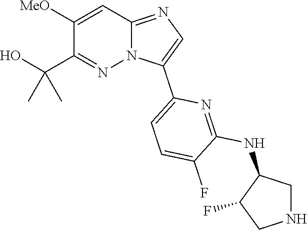 [SMILES: COc1cc2ncc(-c3ccc(F)c(N[C@H]4CNC[C@@H]4F)n3)n2nc1C(C)(C)O] |
| 6a-8 | 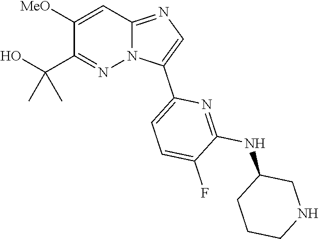 [SMILES: COc1cc2ncc(-c3ccc(F)c(N[C@@H]4CCCNC4)n3)n2nc1C(C)(C)O] |
| 7a-8 | 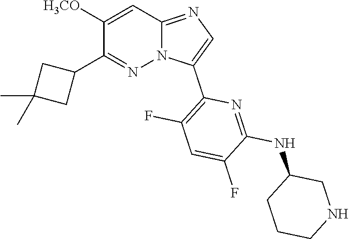 [SMILES: COc1cc2ncc(-c3nc(N[C@@H]4CCCNC4)c(F)cc3F)n2nc1C1CC(C)(C)C1] |
| 8a-8 | 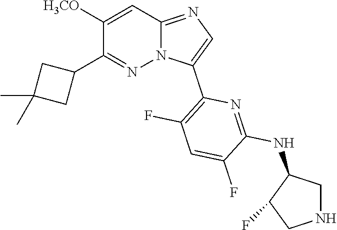 [SMILES: COc1cc2ncc(-c3nc(N[C@H]4CNC[C@@H]4F)c(F)cc3F)n2nc1C1CC(C)(C)C1] |
| 9a-8 | 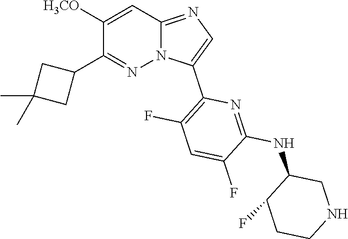 [SMILES: COc1cc2ncc(-c3nc(N[C@H]4CNCC[C@@H]4F)c(F)cc3F)n2nc1C1CC(C)(C)C1] |
| 10a-8 | 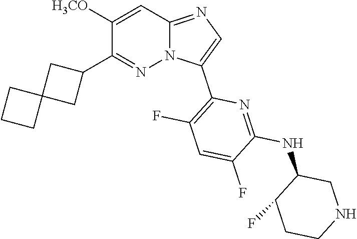 [SMILES: COc1cc2ncc(-c3nc(N[C@H]4CNCC[C@@H]4F)c(F)cc3F)n2nc1C1CC2(CCC2)C1] |
| 11a-8 | 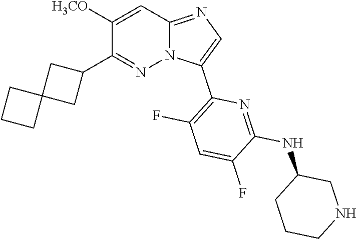 [SMILES: COc1cc2ncc(-c3nc(N[C@@H]4CCCNC4)c(F)cc3F)n2nc1C1CC2(CCC2)C1] |
| 12a-8 | 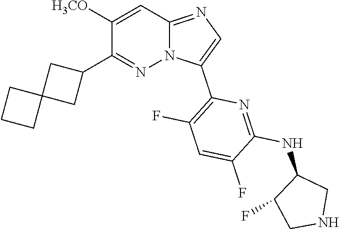 [SMILES: COc1cc2ncc(-c3nc(N[C@H]4CNC[C@@H]4F)c(F)cc3F)n2nc1C1CC2(CCC2)C1] |
| 13a-8 | 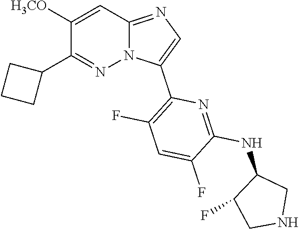 [SMILES: COc1cc2ncc(-c3nc(N[C@H]4CNC[C@@H]4F)c(F)cc3F)n2nc1C1CCC1] |
| 14a-8 | 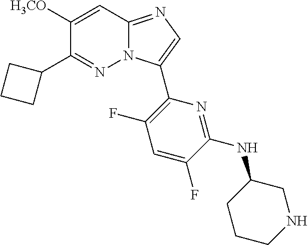 [SMILES: COc1cc2ncc(-c3nc(N[C@@H]4CCCNC4)c(F)cc3F)n2nc1C1CCC1] |
| 15a-8 | 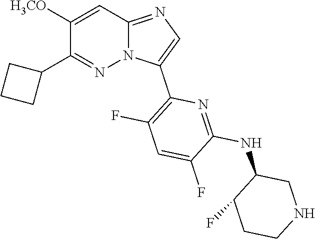 [SMILES: COc1cc2ncc(-c3nc(N[C@H]4CNCC[C@@H]4F)c(F)cc3F)n2nc1C1CCC1] |
| 16a-8 | 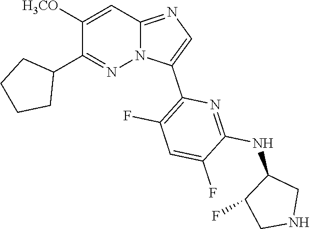 [SMILES: COc1cc2ncc(-c3nc(N[C@H]4CNC[C@@H]4F)c(F)cc3F)n2nc1C1CCCC1] |
| 17a-8 | 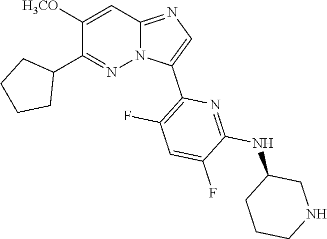 [SMILES: COc1cc2ncc(-c3nc(N[C@@H]4CCCNC4)c(F)cc3F)n2nc1C1CCCC1] |
| 18a-8 | 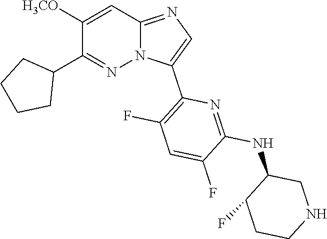 [SMILES: COc1cc2ncc(-c3nc(N[C@H]4CNCC[C@@H]4F)c(F)cc3F)n2nc1C1CCCC1] |
| 19a-8 | 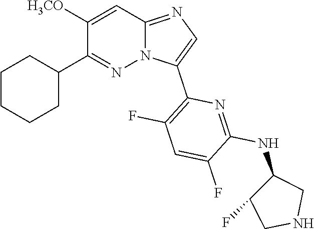 [SMILES: COc1cc2ncc(-c3nc(N[C@H]4CNC[C@@H]4F)c(F)cc3F)n2nc1C1CCCCC1] |
| 20a-8 | 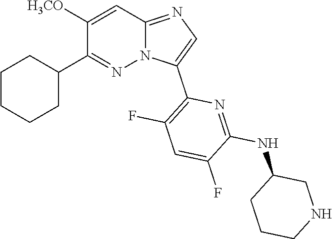 [SMILES: COc1cc2ncc(-c3nc(N[C@@H]4CCCNC4)c(F)cc3F)n2nc1C1CCCCC1] |
| 21a-8 | 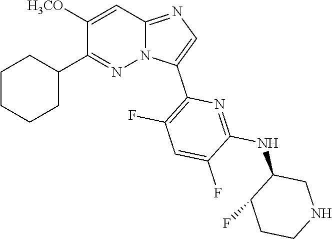 [SMILES: COc1cc2ncc(-c3nc(N[C@H]4CNCC[C@@H]4F)c(F)cc3F)n2nc1C1CCCCC1] |
| 22a-8 | 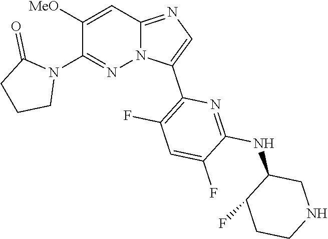 [SMILES: COc1cc2ncc(-c3nc(N[C@H]4CNCC[C@@H]4F)c(F)cc3F)n2nc1N1CCCC1=O] |
| 23a-8 | 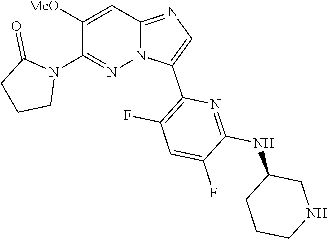 [SMILES: COc1cc2ncc(-c3nc(N[C@@H]4CCCNC4)c(F)cc3F)n2nc1N1CCCC1=O] |
| 24a-8 | 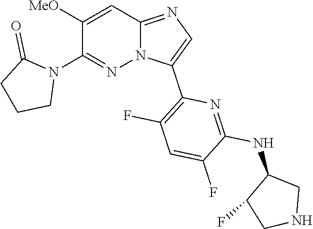 [SMILES: COc1cc2ncc(-c3nc(N[C@H]4CNC[C@@H]4F)c(F)cc3F)n2nc1N1CCCC1=O] |
| 25a-8 | 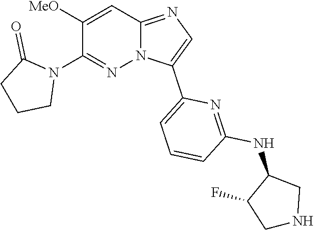 [SMILES: COc1cc2ncc(-c3cccc(N[C@H]4CNC[C@@H]4F)n3)n2nc1N1CCCC1=O] |
| 26a-8 | 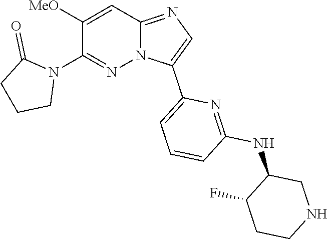 [SMILES: COc1cc2ncc(-c3cccc(N[C@H]4CNCC[C@@H]4F)n3)n2nc1N1CCCC1=O] |
| 27a-8 | 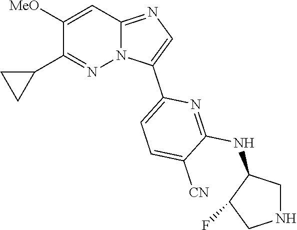 [SMILES: COc1cc2ncc(-c3ccc(C#N)c(N[C@H]4CNC[C@@H]4F)n3)n2nc1C1CC1] |
| 28a-8 | 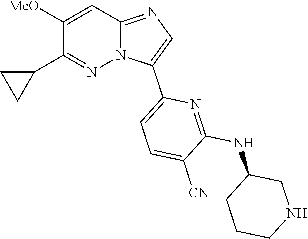 [SMILES: COc1cc2ncc(-c3ccc(C#N)c(N[C@@H]4CCCNC4)n3)n2nc1C1CC1] |
| 29a-8 | 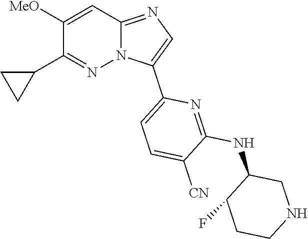 [SMILES: COc1cc2ncc(-c3ccc(C#N)c(N[C@H]4CNCC[C@@H]4F)n3)n2nc1C1CC1] |
| 30a-8 | 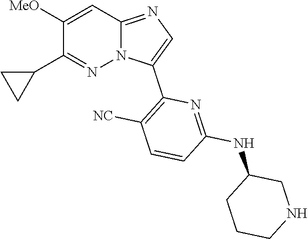 [SMILES: COc1cc2ncc(-c3nc(N[C@@H]4CCCNC4)ccc3C#N)n2nc1C1CC1] |
| 31a-8 | 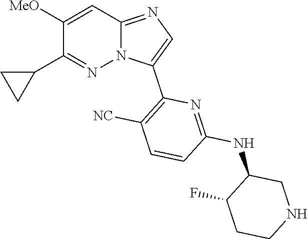 [SMILES: COc1cc2ncc(-c3nc(N[C@H]4CNCC[C@@H]4F)ccc3C#N)n2nc1C1CC1] |
| 32a-8 | 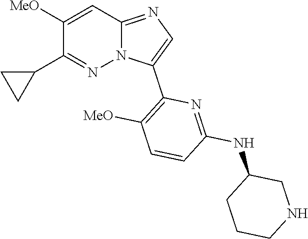 [SMILES: COc1ccc(N[C@@H]2CCCNC2)nc1-c1cnc2cc(OC)c(C3CC3)nn12] |
| 33a-8 | 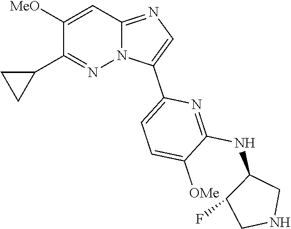 [SMILES: COc1ccc(-c2cnc3cc(OC)c(C4CC4)nn23)nc1N[C@H]1CNC[C@@H]1F] |
| 34a-8 | 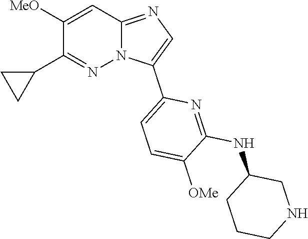 [SMILES: COc1ccc(-c2cnc3cc(OC)c(C4CC4)nn23)nc1N[C@@H]1CCCNC1] |
| 35a-8 | 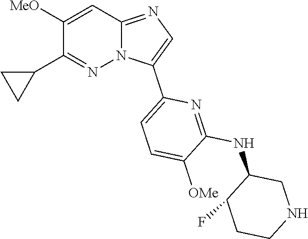 [SMILES: COc1ccc(-c2cnc3cc(OC)c(C4CC4)nn23)nc1N[C@H]1CNCC[C@@H]1F] |
| 36a-8 | 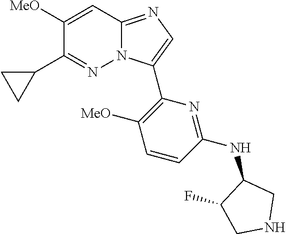 [SMILES: COc1ccc(N[C@H]2CNC[C@@H]2F)nc1-c1cnc2cc(OC)c(C3CC3)nn12] |
| 37a-8 | 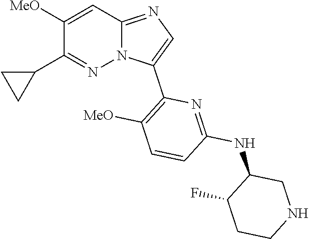 [SMILES: COc1ccc(N[C@H]2CNCC[C@@H]2F)nc1-c1cnc2cc(OC)c(C3CC3)nn12] |
| 38a-8 | 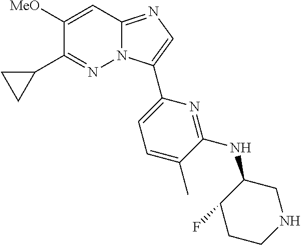 [SMILES: COc1cc2ncc(-c3ccc(C)c(N[C@H]4CNCC[C@@H]4F)n3)n2nc1C1CC1] |
| 39a-8 | 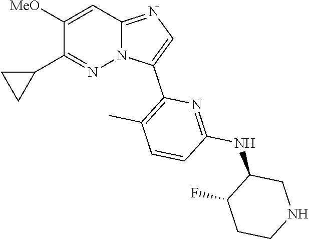 [SMILES: COc1cc2ncc(-c3nc(N[C@H]4CNCC[C@@H]4F)ccc3C)n2nc1C1CC1] |
| 40a-8 | 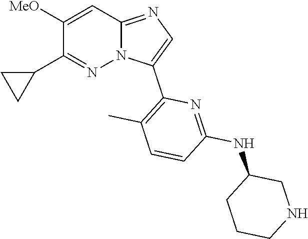 [SMILES: COc1cc2ncc(-c3nc(N[C@@H]4CCCNC4)ccc3C)n2nc1C1CC1] |
| 41a-8 | 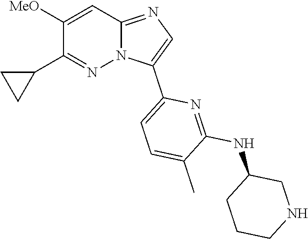 [SMILES: COc1cc2ncc(-c3ccc(C)c(N[C@@H]4CCCNC4)n3)n2nc1C1CC1] |
| 42a-8 | 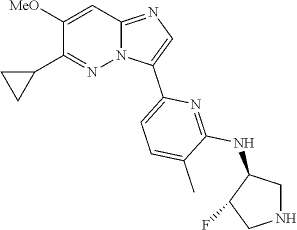 [SMILES: COc1cc2ncc(-c3ccc(C)c(N[C@H]4CNC[C@@H]4F)n3)n2nc1C1CC1] |
| 43a-8 | 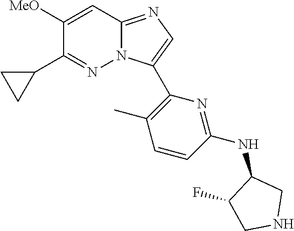 [SMILES: COc1cc2ncc(-c3nc(N[C@H]4CNC[C@@H]4F)ccc3C)n2nc1C1CC1] |
| 44a-8 | 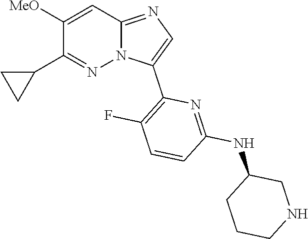 [SMILES: COc1cc2ncc(-c3nc(N[C@@H]4CCCNC4)ccc3F)n2nc1C1CC1] |
| 45a-8 | 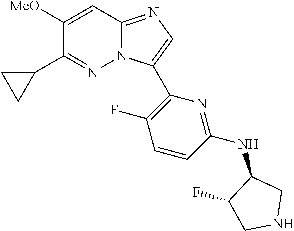 [SMILES: COc1cc2ncc(-c3nc(N[C@H]4CNC[C@@H]4F)ccc3F)n2nc1C1CC1] |
| 46a-8 | 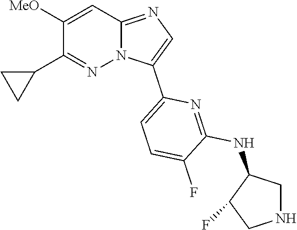 [SMILES: COc1cc2ncc(-c3ccc(F)c(N[C@H]4CNC[C@@H]4F)n3)n2nc1C1CC1] |
| 47a-8 | 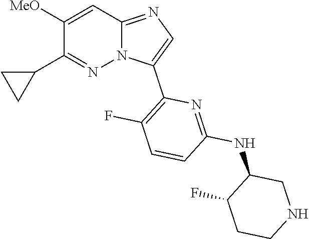 [SMILES: COc1cc2ncc(-c3nc(N[C@H]4CNCC[C@@H]4F)ccc3F)n2nc1C1CC1] |
| 48a-8 | 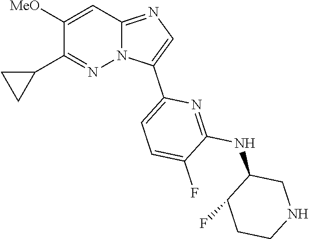 [SMILES: COc1cc2ncc(-c3ccc(F)c(N[C@H]4CNCC[C@@H]4F)n3)n2nc1C1CC1] |
| 49a-8 | 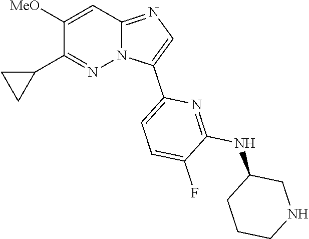 [SMILES: COc1cc2ncc(-c3ccc(F)c(N[C@@H]4CCCNC4)n3)n2nc1C1CC1] |
| 50a-8 | 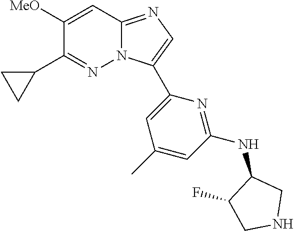 [SMILES: COc1cc2ncc(-c3cc(C)cc(N[C@H]4CNC[C@@H]4F)n3)n2nc1C1CC1] |
| 51a-8 | 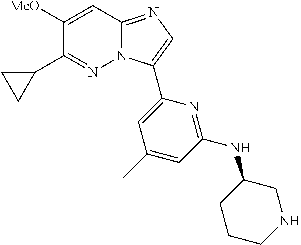 [SMILES: COc1cc2ncc(-c3cc(C)cc(N[C@@H]4CCCNC4)n3)n2nc1C1CC1] |
| 52a-8 | 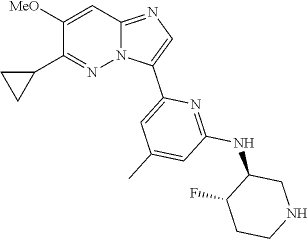 [SMILES: COc1cc2ncc(-c3cc(C)cc(N[C@H]4CNCC[C@@H]4F)n3)n2nc1C1CC1] |
| 53a-8 | 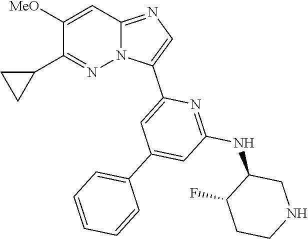 [SMILES: COc1cc2ncc(-c3cc(-c4ccccc4)cc(N[C@H]4CNCC[C@@H]4F)n3)n2nc1C1CC1] |
| 54a-8 | 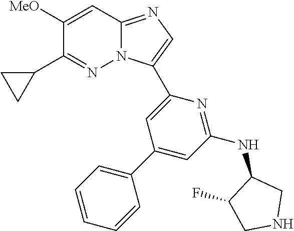 [SMILES: COc1cc2ncc(-c3cc(-c4ccccc4)cc(N[C@H]4CNC[C@@H]4F)n3)n2nc1C1CC1] |
| 55a-8 | 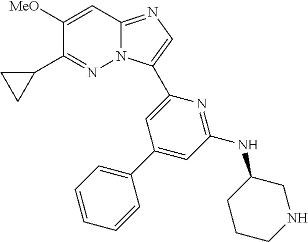 [SMILES: COc1cc2ncc(-c3cc(-c4ccccc4)cc(N[C@@H]4CCCNC4)n3)n2nc1C1CC1] |
| 56a-8 | 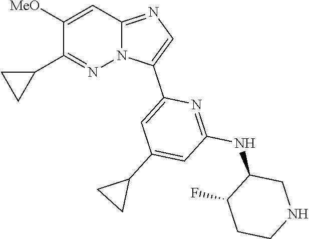 [SMILES: COc1cc2ncc(-c3cc(C4CC4)cc(N[C@H]4CNCC[C@@H]4F)n3)n2nc1C1CC1] |
| 57a-8 | 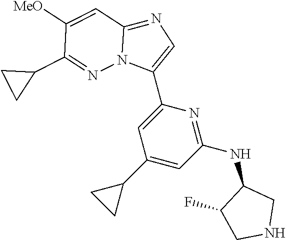 [SMILES: COc1cc2ncc(-c3cc(C4CC4)cc(N[C@H]4CNC[C@@H]4F)n3)n2nc1C1CC1] |
| 58a-8 | 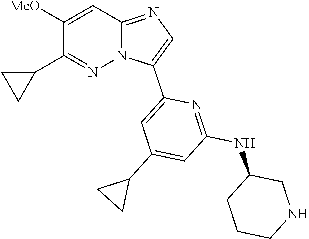 [SMILES: COc1cc2ncc(-c3cc(C4CC4)cc(N[C@@H]4CCCNC4)n3)n2nc1C1CC1] |
| 59a-8 | 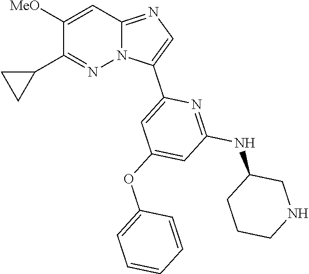 [SMILES: COc1cc2ncc(-c3cc(Oc4ccccc4)cc(N[C@@H]4CCCNC4)n3)n2nc1C1CC1] |
| 60a-8 | 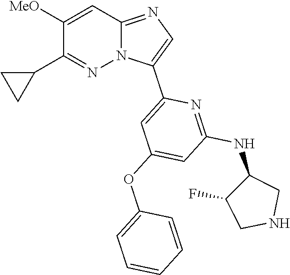 [SMILES: COc1cc2ncc(-c3cc(Oc4ccccc4)cc(N[C@H]4CNC[C@@H]4F)n3)n2nc1C1CC1] |
| 61a-8 | 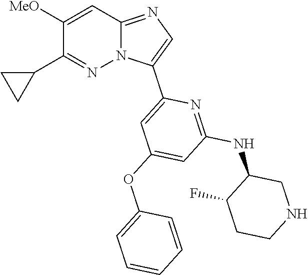 [SMILES: COc1cc2ncc(-c3cc(Oc4ccccc4)cc(N[C@H]4CNCC[C@@H]4F)n3)n2nc1C1CC1] |
| 62a-8 | 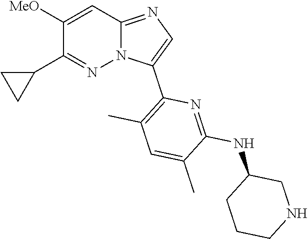 [SMILES: COc1cc2ncc(-c3nc(N[C@@H]4CCCNC4)c(C)cc3C)n2nc1C1CC1] |
| 63a-8 | 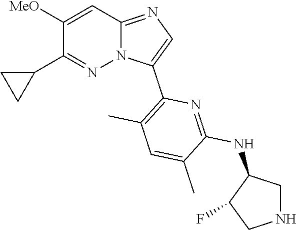 [SMILES: COc1cc2ncc(-c3nc(N[C@H]4CNC[C@@H]4F)c(C)cc3C)n2nc1C1CC1] |
| 64a-8 | 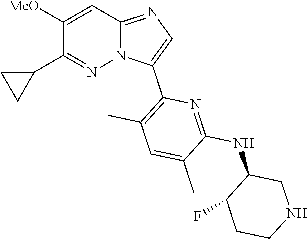 [SMILES: COc1cc2ncc(-c3nc(N[C@H]4CNCC[C@@H]4F)c(C)cc3C)n2nc1C1CC1] |
| 65a-8 | 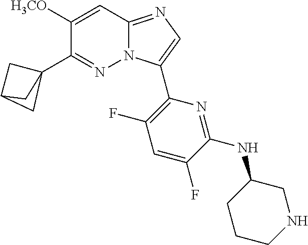 [SMILES: COc1cc2ncc(-c3nc(N[C@@H]4CCCNC4)c(F)cc3F)n2nc1C12CC(C1)C2] |
| 66a-8 | 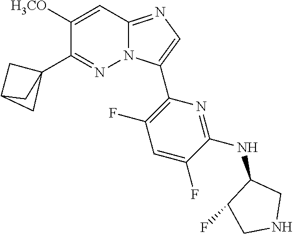 [SMILES: COc1cc2ncc(-c3nc(N[C@H]4CNC[C@@H]4F)c(F)cc3F)n2nc1C12CC(C1)C2] |
| 67a-8 | 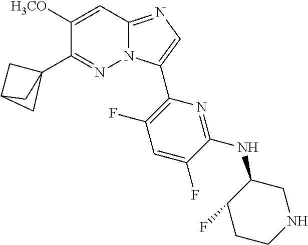 [SMILES: COc1cc2ncc(-c3nc(N[C@H]4CNCC[C@@H]4F)c(F)cc3F)n2nc1C12CC(C1)C2] |
| 68a-8 | 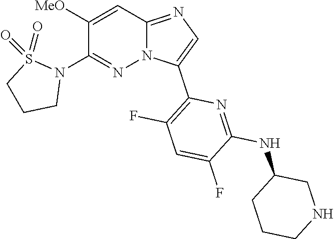 [SMILES: COc1cc2ncc(-c3nc(N[C@@H]4CCCNC4)c(F)cc3F)n2nc1N1CCCS1(=O)=O] |
| 69a-8 | 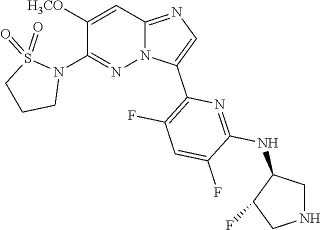 [SMILES: COc1cc2ncc(-c3nc(N[C@H]4CNC[C@@H]4F)c(F)cc3F)n2nc1N1CCCS1(=O)=O] |
| 70a-8 | 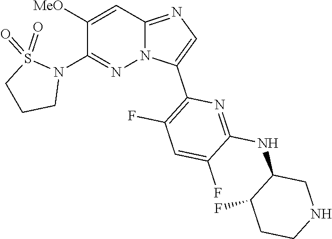 [SMILES: COc1cc2ncc(-c3nc(N[C@H]4CNCC[C@@H]4F)c(F)cc3F)n2nc1N1CCCS1(=O)=O] |
| 71a-8 | 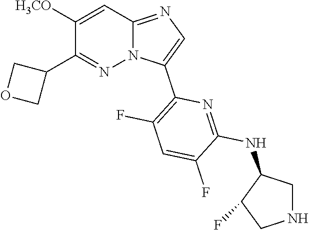 [SMILES: COc1cc2ncc(-c3nc(N[C@H]4CNC[C@@H]4F)c(F)cc3F)n2nc1C1COC1] |
| 72a-8 | 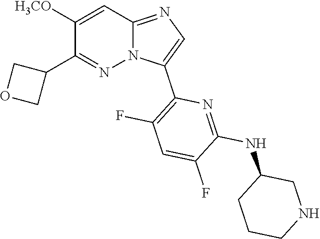 [SMILES: COc1cc2ncc(-c3nc(N[C@@H]4CCCNC4)c(F)cc3F)n2nc1C1COC1] |
| 73a-8 | 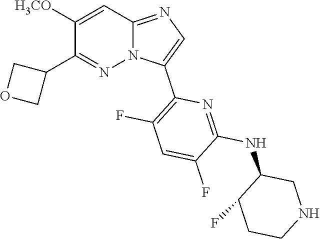 [SMILES: COc1cc2ncc(-c3nc(N[C@H]4CNCC[C@@H]4F)c(F)cc3F)n2nc1C1COC1] |
| 74a-8 | 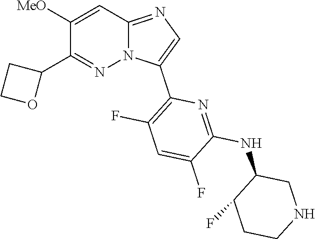 [SMILES: COc1cc2ncc(-c3nc(N[C@H]4CNCC[C@@H]4F)c(F)cc3F)n2nc1C1CCO1] |
| 75a-8 | 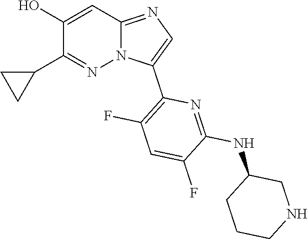 [SMILES: Oc1cc2ncc(-c3nc(N[C@@H]4CCCNC4)c(F)cc3F)n2nc1C1CC1] |
| 76a-8 | 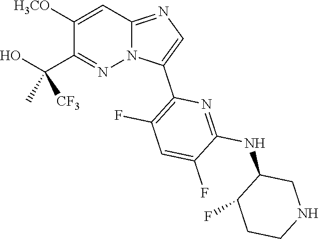 [SMILES: COc1cc2ncc(-c3nc(N[C@H]4CNCC[C@@H]4F)c(F)cc3F)n2nc1[C@](C)(O)C(F)(F)F] |
| 77a-8 | 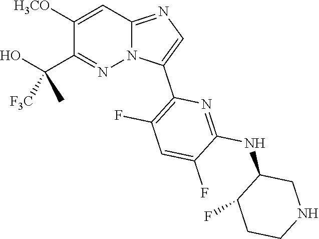 [SMILES: COc1cc2ncc(-c3nc(N[C@H]4CNCC[C@@H]4F)c(F)cc3F)n2nc1[C@@](C)(O)C(F)(F)F] |
| 78a-8 | 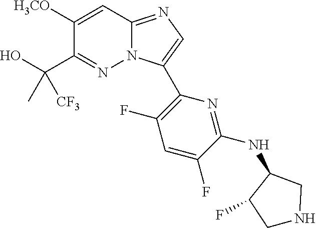 [SMILES: COc1cc2ncc(-c3nc(N[C@H]4CNC[C@@H]4F)c(F)cc3F)n2nc1C(C)(O)C(F)(F)F] |
| 79a-8 | 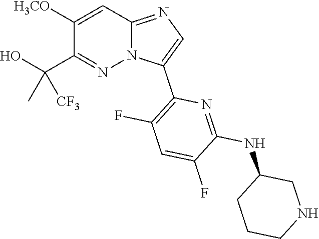 [SMILES: COc1cc2ncc(-c3nc(N[C@@H]4CCCNC4)c(F)cc3F)n2nc1C(C)(O)C(F)(F)F] |
| 80a-8 | 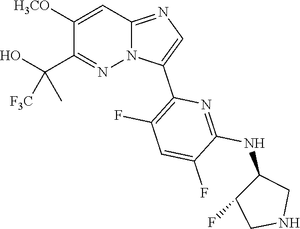 [SMILES: COc1cc2ncc(-c3nc(N[C@H]4CNC[C@@H]4F)c(F)cc3F)n2nc1C(C)(O)C(F)(F)F] |
| 81a-8 | 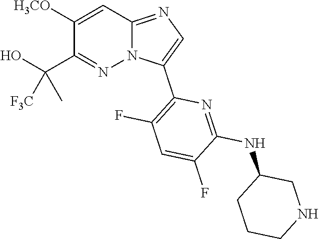 [SMILES: COc1cc2ncc(-c3nc(N[C@@H]4CCCNC4)c(F)cc3F)n2nc1C(C)(O)C(F)(F)F] |
| 82a-8 | 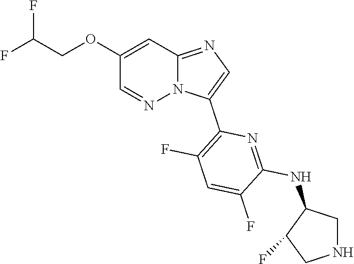 [SMILES: Fc1cc(F)c(-c2cnc3cc(OCC(F)F)cnn23)nc1N[C@H]1CNC[C@@H]1F] |
| 83a-8 | 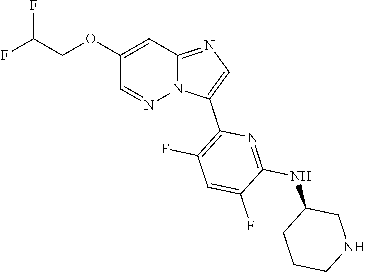 [SMILES: Fc1cc(F)c(-c2cnc3cc(OCC(F)F)cnn23)nc1N[C@@H]1CCCNC1] |
| 84a-8 | 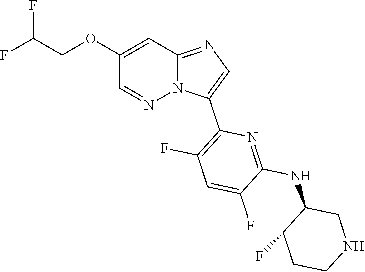 [SMILES: Fc1cc(F)c(-c2cnc3cc(OCC(F)F)cnn23)nc1N[C@H]1CNCC[C@@H]1F] |

In one embodiment, the compound is a compound of Formula (1-5009):

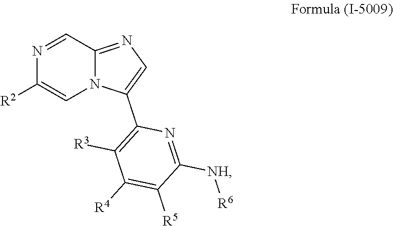

or a salt, ester, solvate, optical isomer, geometric isomer, or salt of an isomer thereof; wherein:
R², R³, R⁴, and R⁵ are each independently selected from H, halogen, hydroxy, oxo (═O), —CN, amino, amido, —O-aryl, methanoyl (—COH), carboxy (—CO₂H), C₁-C₇ alkyl, C₂-C₇ alkenyl, C₂-C₇ alkynyl, C₁-C₇ heteroalkyl, C₁-C₇ alkoxy, cycloalkyl, spiro-fused cycloalkyl, heterocyclyl, aryl, heteroaryl, or fused ring heteroaryl, wherein amino, amido, —O-aryl, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more of halogen, hydroxy, oxo, methanoyl (—COH), carboxy (—CO₂H), nitro (—NO₂), —NH₂, —NHCH₃, —N(CH₃)₂, cyano (—CN), ethynyl (—CCH), propynyl, sulfo (—SO₃H), heterocyclyl, aryl, heteroaryl, pyrrolyl, piperidyl, piperazinyl, morpholinyl, —CO-morpholin-4-yl, —CONH₂, —CONHCH₃, —CON(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl, wherein two adjacent optional substituents can bond or fuse to form a ring;
R⁶ is selected from

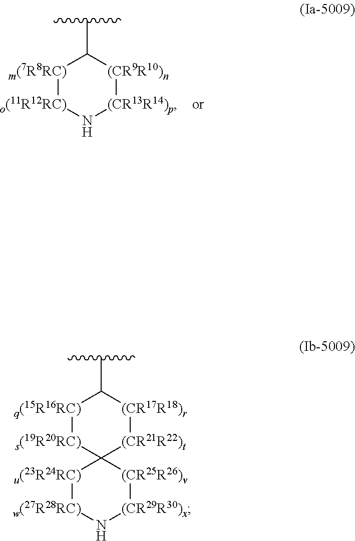

R⁷, R⁸, R⁹, R¹⁰, R, R¹², R¹³, and R¹⁴ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen and/or C₁-C₆ alkyl;
R¹⁵, R¹⁶, R¹⁷, R¹⁸, R¹⁹, R²⁰, R²¹, R²², R²³, R²⁴, R²⁵, R²⁶, R²⁷, R²⁸, R²⁹, and R³⁰ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen and/or C₁-C₆ alkyl; and
m, n, o, p, q, r, s, t, u, v, w, and x are each independently selected from 0, 1, 2, 3, 4, or 5; where q+r+s+t is at least 1, and where u+v+w+x is at least 1.

In one embodiment, the compound of Formula (1-5009) is a compound of Formula (IIf-5009):

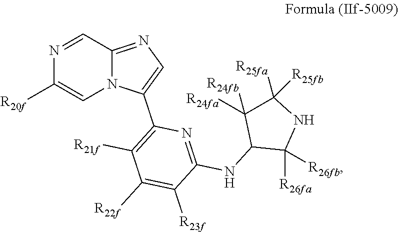

or a salt, ester, solvate, optical isomer, geometric isomer, or salt of an isomer thereof; wherein:
R_{20f} is selected from H, halogen, C₁-C₆ alkyl, C₁-C₆ alkoxy, C₃-C₆ cycloalkyl, —O—(CH₂)a-(C₃-C₆ cycloalkyl), and C₃-C₉ heterocyclyl, wherein C₁-C₆ alkyl and C₁-C₆ alkoxy are each optionally substituted with one or more substituents selected from —OH and halogen, wherein C₃-C₆ cycloalkyl is optionally substituted with one or more substituents selected from C₁-C₆ alkyl and halogen, and wherein C₃-C₉ heterocyclyl is optionally substituted with one or more substituents selected from halogen, C₁-C₆ alkyl, C₁-C₆ alkoxy, —OH, and ═O wherein two adjacent optional substituents can bond or fuse to form a ring;
R_{21f}, R_{22f}, and R_{23f} are each independently selected from H and halogen;
R_{24fa}, R_{24fb}, R_{25fa}, R_{25fb}, R_{26fa}, and R_{26fb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
a is selected from 0, 1, 2, 3, 4, 5, and 6.

In one embodiment, the compound of Formula (1-5009) is a compound of Formula (IIg-5009):

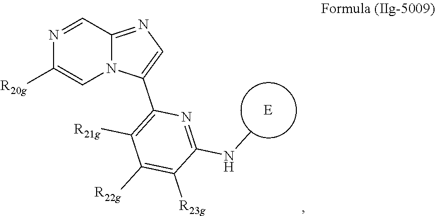

or a salt, ester, solvate, optical isomer, geometric isomer, or salt of an isomer thereof, wherein:

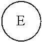

is selected from

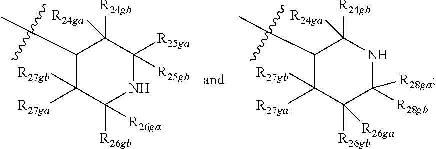

R_{20g} is selected from H, C₁-C₆ alkyl, C₁-C₆ alkoxy, C₃-C₆ cycloalkyl, and C₃-C₉ heterocyclyl, wherein C₁-C₆ alkyl and C₁-C₆ alkoxy are each optionally substituted with one or more substituents selected from —OH and halogen, wherein C₃-C₆ cycloalkyl is optionally substituted with one or more substituents selected from C₁-C₆ alkyl and halogen, and wherein C₃-C₉ heterocyclyl is optionally substituted with one or more substituents selected from halogen, C₁-C₆ alkyl, C₃-C₆-cycloalkyl, —OH, and ═O;
R_{21g}, R_{22g}, and R_{23g} are each independently selected from H and halogen; and R_{24ga}, R_{24gb}, R_{25ga}, R_{25gb}, R_{26ga}, R_{26gb}, R_{27ga}, R_{27gb}, R_{28ga}, and R_{28gb} are each independently selected from H, halogen, —OH, and C₁-C₆ alkyl.

In one embodiment, the compound of Formula (1-5009) is a compound of Formula (IIh-5009):

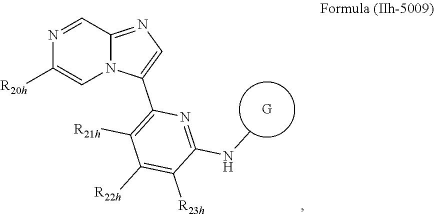

or a salt, ester, solvate, optical isomer, geometric isomer, or salt of an isomer thereof; wherein:

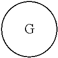

is selected from

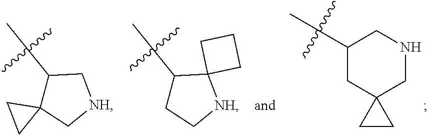 [SMILES: *C1CNCC12CC2 ; *C1CCNC12CCC2 ; *C1CNCC2(CC2)C1]

R₂₀ₕ is selected from H, C₁-C₆ alkyl, C₁-C₆ alkoxy, and C₃-C₆ cycloalkyl, wherein C₁-C₆ alkyl and C₁-C₆ alkoxy are each optionally substituted with one or more substituents selected from halogen and —OH, and wherein C₃-C₆ cycloalkyl is optionally substituted with one or more substituents selected from C₁-C₆ alkyl and halogen; and
R₂₁ₕ, R₂₂ₕ, and R₂₃ₕ are each independently selected from H and halogen.

In one embodiment, the compound of Formula (1-5009) is a compound of Formula (IIi-5009):

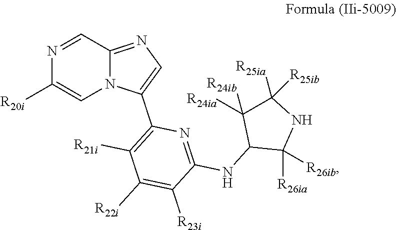

or a salt, ester, solvate, optical isomer, geometric isomer, or salt of an isomer thereof; wherein:
R₂₀ᵢ is selected from C₁-C₆ alkyl and C₁-C₆ alkoxy, wherein C₁-C₆ alkyl and C₁-C₆ alkoxy are each optionally substituted with one or more substituents selected from —OH and halogen;
R₂₁ᵢ, R₂₂ᵢ, and R₂₃ᵢ are each independently selected from H and halogen; and
R₂₄ᵢₐ, R_{24ib}, R₂₅ᵢₐ, R_{25sb}, R₂₆ᵢₐ, and R_{26ib} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms.

In one embodiment, the compound of Formula (1-5009) is a compound of Formula (IIj-5009):

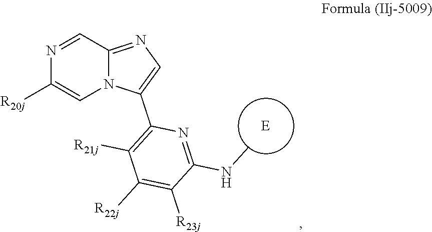

or a salt, ester, solvate, optical isomer, geometric isomer, or salt of an isomer thereof, wherein:

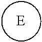

is selected from

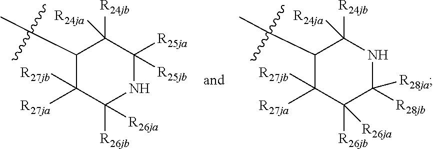

R₂₀ⱼ is selected from C₁-C₆ alkyl and C₁-C₆ alkoxy, wherein C₁-C₆ alkyl and C₁-C₆ alkoxy are each optionally substituted with one or more substituents selected from —OH and halogen;
R₂₁ⱼ, R₂₂ⱼ, and R₂₃ⱼ are each independently selected from H and halogen; and
R₂₄ⱼₐ, R_{24jb}, R₂₅ⱼₐ, R_{25jb}, R₂₆ⱼₐ, R_{26jb}, R₂₇ⱼₐ, R_{27jb}, R₂₈ⱼₐ, and R_{28jb} are each independently selected from H, halogen, —OH, and C₁-C₆ alkyl.

In one embodiment, the compound of Formula (1-5009) is selected from Compounds 1-9 to 138-9.

| # | Structure |
|---|---|
| 1-9 | 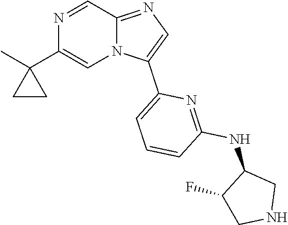 [SMILES: CC1(c2cn3c(-c4cccc(N[C@H]5CNC[C@@H]5F)n4)cnc3cn2)CC1] |
| 2-9 | 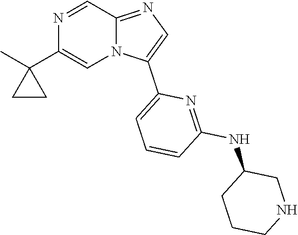 [SMILES: CC1(c2cn3c(-c4cccc(N[C@@H]5CCCNC5)n4)cnc3cn2)CC1] |
| 3-9 | 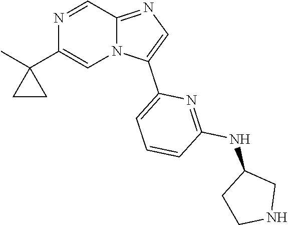 [SMILES: CC1(c2cn3c(-c4cccc(N[C@@H]5CCNC5)n4)cnc3cn2)CC1] |
| 4-9 | 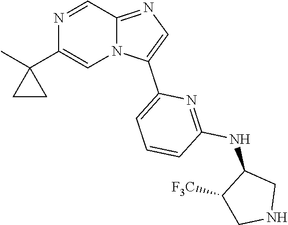 [SMILES: CC1(c2cn3c(-c4cccc(N[C@H]5CNC[C@@H]5C(F)(F)F)n4)cnc3cn2)CC1] |
| 5-9 | 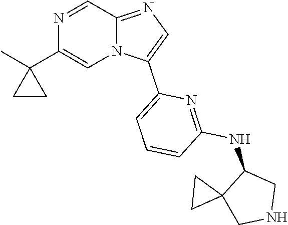 [SMILES: CC1(c2cn3c(-c4cccc(N[C@H]5CNCC56CC6)n4)cnc3cn2)CC1] |
| 6-9 | 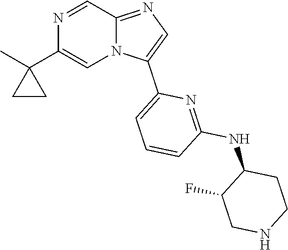 [SMILES: CC1(c2cn3c(-c4cccc(N[C@H]5CCNC[C@@H]5F)n4)cnc3cn2)CC1] |
| 7-9 | 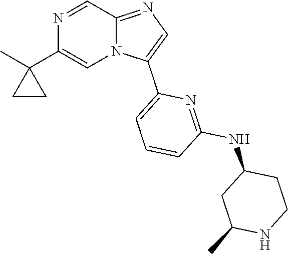 [SMILES: C[C@H]1C[C@@H](Nc2cccc(-c3cnc4cnc(C5(C)CC5)cn34)n2)CCN1] |
| 8-9 | 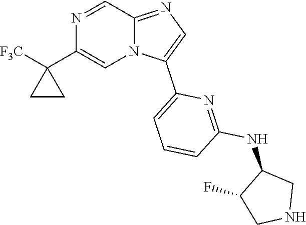 [SMILES: F[C@H]1CNC[C@@H]1Nc1cccc(-c2cnc3cnc(C4(C(F)(F)F)CC4)cn23)n1] |
| 9-9 | 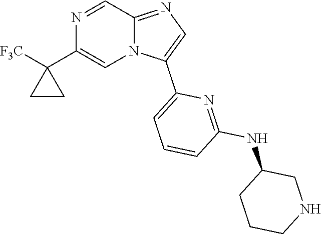 [SMILES: FC(F)(F)C1(c2cn3c(-c4cccc(N[C@@H]5CCCNC5)n4)cnc3cn2)CC1] |
| 10-9 | 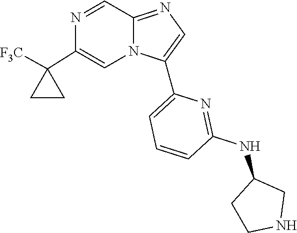 [SMILES: FC(F)(F)C1(c2cn3c(-c4cccc(N[C@@H]5CCNC5)n4)cnc3cn2)CC1] |
| 11-9 | 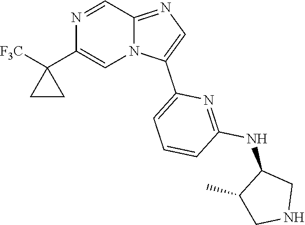 [SMILES: C[C@H]1CNC[C@@H]1Nc1cccc(-c2cnc3cnc(C4(C(F)(F)F)CC4)cn23)n1] |
| 12-9 | 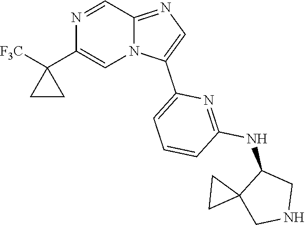 [SMILES: FC(F)(F)C1(c2cn3c(-c4cccc(N[C@H]5CNCC56CC6)n4)cnc3cn2)CC1] |
| 13-9 | 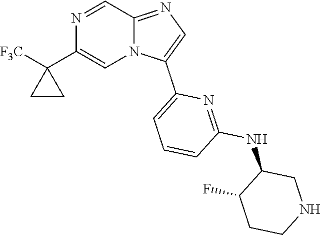 [SMILES: F[C@H]1CCNC[C@@H]1Nc1cccc(-c2cnc3cnc(C4(C(F)(F)F)CC4)cn23)n1] |
| 14-9 | 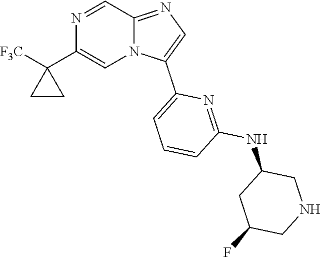 [SMILES: F[C@@H]1CNC[C@H](Nc2cccc(-c3cnc4cnc(C5(C(F)(F)F)CC5)cn34)n2)C1] |
| 15-9 | 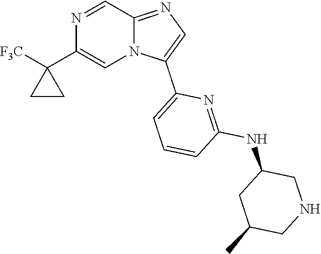 [SMILES: C[C@@H]1CNC[C@H](Nc2cccc(-c3cnc4cnc(C5(C(F)(F)F)CC5)cn34)n2)C1] |
| 16-9 | 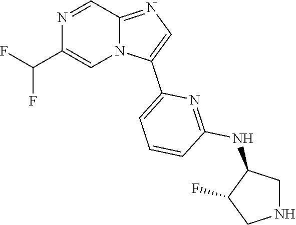 [SMILES: FC(F)c1cn2c(-c3cccc(N[C@H]4CNC[C@@H]4F)n3)cnc2cn1] |
| 17-9 | 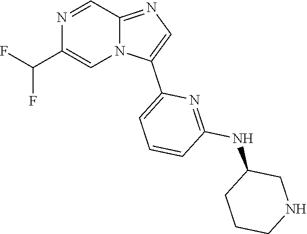 [SMILES: FC(F)c1cn2c(-c3cccc(N[C@@H]4CCCNC4)n3)cnc2cn1] |
| 18-9 | 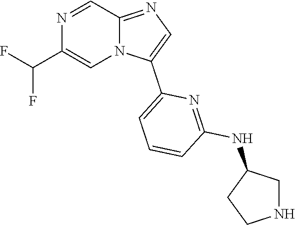 [SMILES: FC(F)c1cn2c(-c3cccc(N[C@@H]4CCNC4)n3)cnc2cn1] |
| 19-9 | 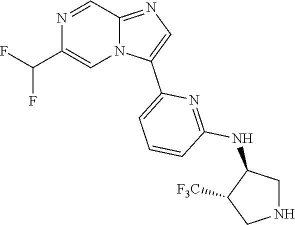 [SMILES: FC(F)c1cn2c(-c3cccc(N[C@H]4CNC[C@@H]4C(F)(F)F)n3)cnc2cn1] |
| 20-9 | 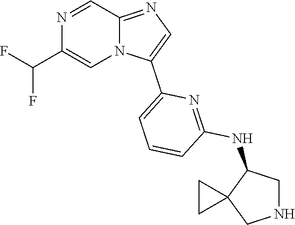 [SMILES: FC(F)c1cn2c(-c3cccc(N[C@H]4CNCC45CC5)n3)cnc2cn1] |
| 21-9 | 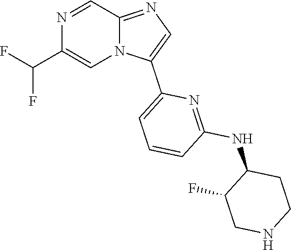 [SMILES: FC(F)c1cn2c(-c3cccc(N[C@H]4CCNC[C@@H]4F)n3)cnc2cn1] |
| 22-9 | 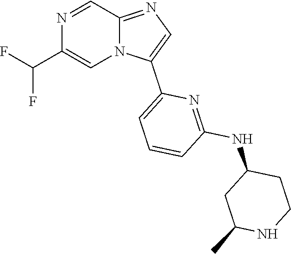 [SMILES: C[C@H]1C[C@@H](Nc2cccc(-c3cnc4cnc(C(F)F)cn34)n2)CCN1] |
| 23-9 | 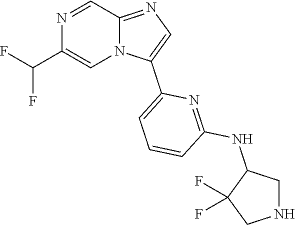 [SMILES: FC(F)c1cn2c(-c3cccc(NC4CNCC4(F)F)n3)cnc2cn1] |
| 24-9 | 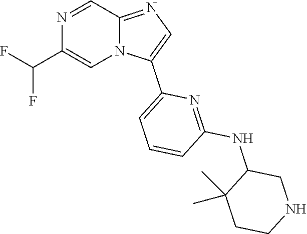 [SMILES: CC1(C)CCNCC1Nc1cccc(-c2cnc3cnc(C(F)F)cn23)n1] |
| 25-9 | 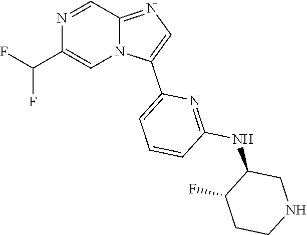 [SMILES: FC(F)c1cn2c(-c3cccc(N[C@H]4CNCC[C@@H]4F)n3)cnc2cn1] |
| 26-9 | 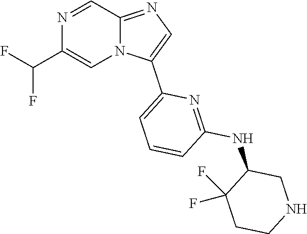 [SMILES: FC(F)c1cn2c(-c3cccc(N[C@H]4CNCCC4(F)F)n3)cnc2cn1] |
| 27-9 | 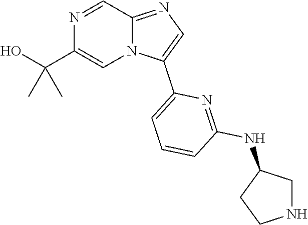 [SMILES: CC(C)(O)c1cn2c(-c3cccc(N[C@@H]4CCNC4)n3)cnc2cn1] |
| 28-9 | 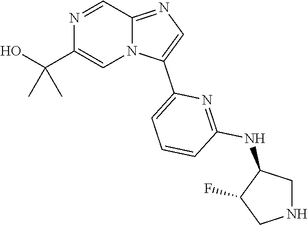 [SMILES: CC(C)(O)c1cn2c(-c3cccc(N[C@H]4CNC[C@@H]4F)n3)cnc2cn1] |
| 29-9 | 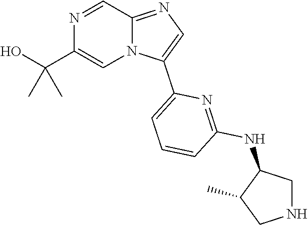 [SMILES: C[C@H]1CNC[C@@H]1Nc1cccc(-c2cnc3cnc(C(C)(C)O)cn23)n1] |
| 30-9 | 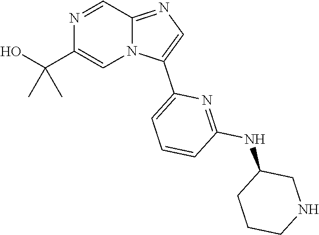 [SMILES: CC(C)(O)c1cn2c(-c3cccc(N[C@@H]4CCCNC4)n3)cnc2cn1] |
| 31-9 | 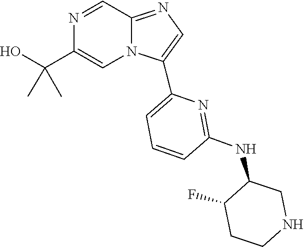 [SMILES: CC(C)(O)c1cn2c(-c3cccc(N[C@H]4CNCC[C@@H]4F)n3)cnc2cn1] |
| 32-9 | 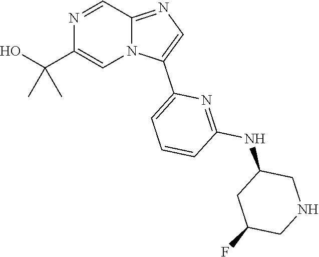 [SMILES: CC(C)(O)c1cn2c(-c3cccc(N[C@H]4CNC[C@@H](F)C4)n3)cnc2cn1] |
| 33-9 | 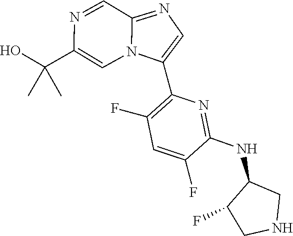 [SMILES: CC(C)(O)c1cn2c(-c3nc(N[C@H]4CNC[C@@H]4F)c(F)cc3F)cnc2cn1] |
| 34-9 | 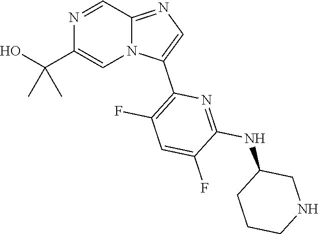 [SMILES: CC(C)(O)c1cn2c(-c3nc(N[C@@H]4CCCNC4)c(F)cc3F)cnc2cn1] |
| 35-9 | 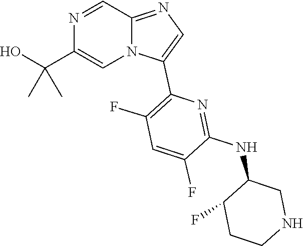 [SMILES: CC(C)(O)c1cn2c(-c3nc(N[C@H]4CNCC[C@@H]4F)c(F)cc3F)cnc2cn1] |
| 36-9 | 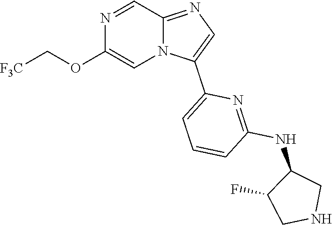 [SMILES: F[C@H]1CNC[C@@H]1Nc1cccc(-c2cnc3cnc(OCC(F)(F)F)cn23)n1] |
| 37-9 | 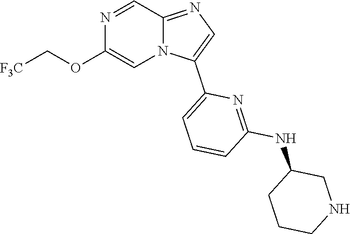 [SMILES: FC(F)(F)COc1cn2c(-c3cccc(N[C@@H]4CCCNC4)n3)cnc2cn1] |
| 38-9 | 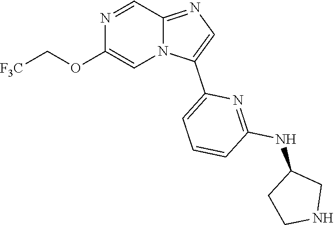 [SMILES: FC(F)(F)COc1cn2c(-c3cccc(N[C@@H]4CCNC4)n3)cnc2cn1] |
| 39-9 | 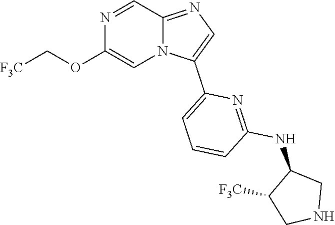 [SMILES: FC(F)(F)COc1cn2c(-c3cccc(N[C@H]4CNC[C@@H]4C(F)(F)F)n3)cnc2cn1] |
| 40-9 | 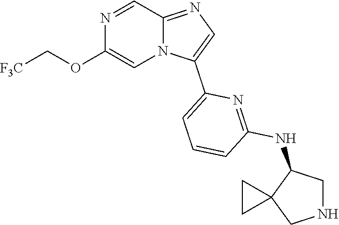 [SMILES: FC(F)(F)COc1cn2c(-c3cccc(N[C@H]4CNCC45CC5)n3)cnc2cn1] |
| 41-9 | 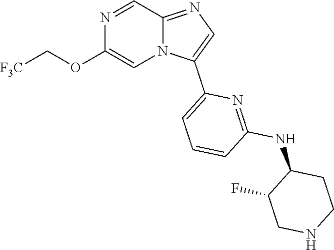 [SMILES: F[C@H]1CNCC[C@@H]1Nc1cccc(-c2cnc3cnc(OCC(F)(F)F)cn23)n1] |
| 42-9 | 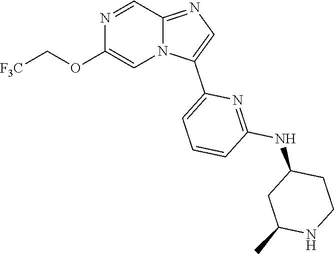 [SMILES: C[C@H]1C[C@@H](Nc2cccc(-c3cnc4cnc(OCC(F)(F)F)cn34)n2)CCN1] |
| 43-9 | 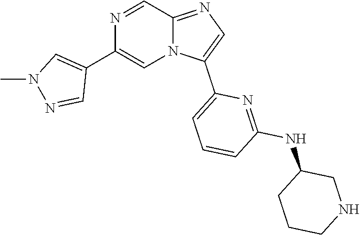 [SMILES: Cn1cc(-c2cn3c(-c4cccc(N[C@@H]5CCCNC5)n4)cnc3cn2)cn1] |
| 44-9 | 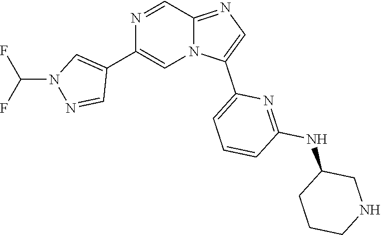 [SMILES: FC(F)n1cc(-c2cn3c(-c4cccc(N[C@@H]5CCCNC5)n4)cnc3cn2)cn1] |
| 45-9 | 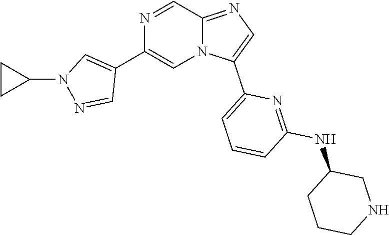 [SMILES: c1cc(N[C@@H]2CCCNC2)nc(-c2cnc3cnc(-c4cnn(C5CC5)c4)cn23)c1] |
| 46-9 | 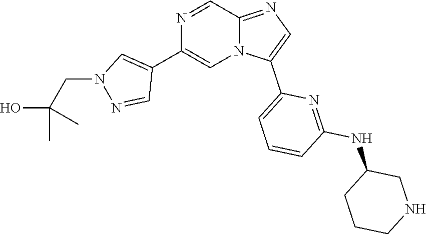 [SMILES: CC(C)(O)Cn1cc(-c2cn3c(-c4cccc(N[C@@H]5CCCNC5)n4)cnc3cn2)cn1] |
| 47-9 | 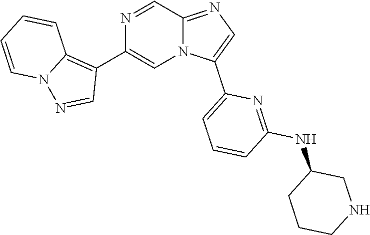 [SMILES: c1cc(N[C@@H]2CCCNC2)nc(-c2cnc3cnc(-c4cnn5ccccc45)cn23)c1] |
| 48-9 | 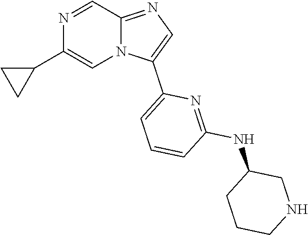 [SMILES: c1cc(N[C@@H]2CCCNC2)nc(-c2cnc3cnc(C4CC4)cn23)c1] |
| 49-9 | 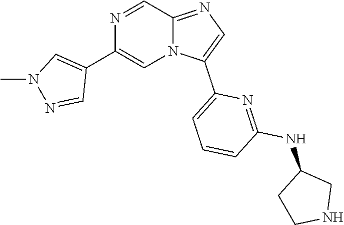 [SMILES: Cn1cc(-c2cn3c(-c4cccc(N[C@@H]5CCNC5)n4)cnc3cn2)cn1] |
| 50-9 | 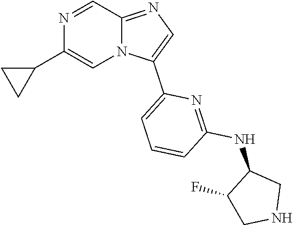 [SMILES: F[C@H]1CNC[C@@H]1Nc1cccc(-c2cnc3cnc(C4CC4)cn23)n1] |
| 51-9 | 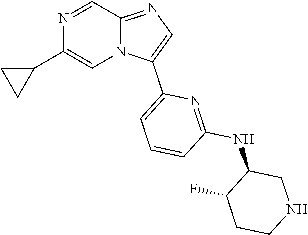 [SMILES: F[C@H]1CCNC[C@@H]1Nc1cccc(-c2cnc3cnc(C4CC4)cn23)n1] |
| 52-9 | 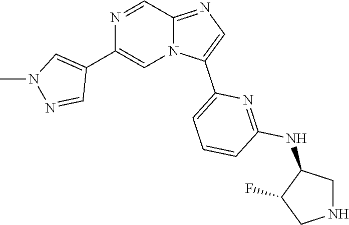 [SMILES: Cn1cc(-c2cn3c(-c4cccc(N[C@H]5CNC[C@@H]5F)n4)cnc3cn2)cn1] |
| 53-9 | 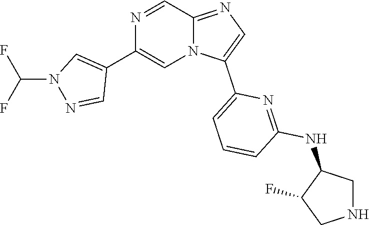 [SMILES: FC(F)n1cc(-c2cn3c(-c4cccc(N[C@H]5CNC[C@@H]5F)n4)cnc3cn2)cn1] |
| 54-9 | 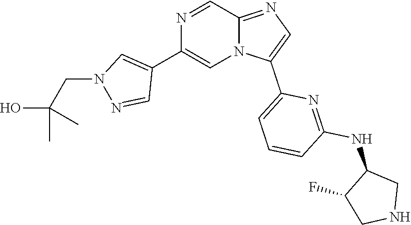 [SMILES: CC(C)(O)Cn1cc(-c2cn3c(-c4cccc(N[C@H]5CNC[C@@H]5F)n4)cnc3cn2)cn1] |
| 55-9 | 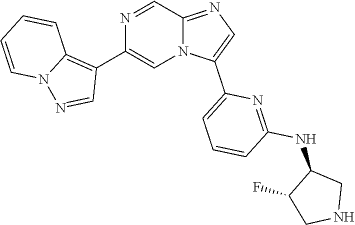 [SMILES: F[C@H]1CNC[C@@H]1Nc1cccc(-c2cnc3cnc(-c4cnn5ccccc45)cn23)n1] |
| 56-9 | 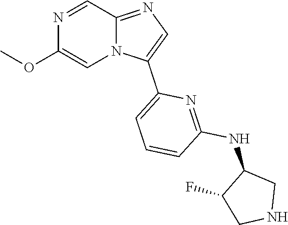 [SMILES: COc1cn2c(-c3cccc(N[C@H]4CNC[C@@H]4F)n3)cnc2cn1] |
| 57 | 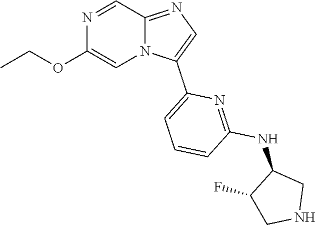 [SMILES: CCOc1cn2c(-c3cccc(N[C@H]4CNC[C@@H]4F)n3)cnc2cn1] |
| 58-9 | 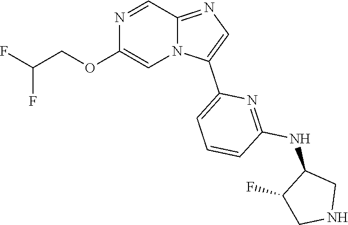 [SMILES: FC(F)COc1cn2c(-c3cccc(N[C@H]4CNC[C@@H]4F)n3)cnc2cn1] |
| 59-9 | 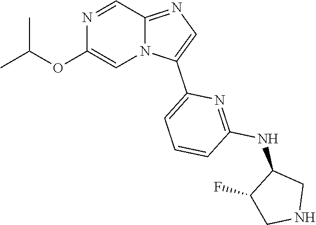 [SMILES: CC(C)Oc1cn2c(-c3cccc(N[C@H]4CNC[C@@H]4F)n3)cnc2cn1] |
| 60-9 | 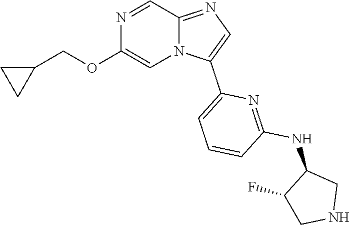 [SMILES: F[C@H]1CNC[C@@H]1Nc1cccc(-c2cnc3cnc(OCC4CC4)cn23)n1] |
| 61-9 | 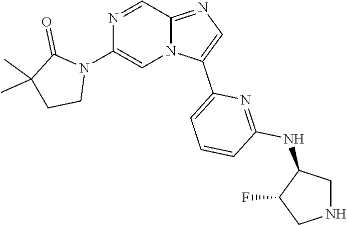 [SMILES: CC1(C)CCN(c2cn3c(-c4cccc(N[C@H]5CNC[C@@H]5F)n4)cnc3cn2)C1=O] |
| 62-9 | 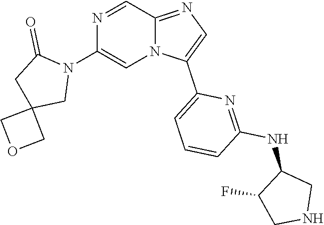 [SMILES: O=C1CC2(COC2)CN1c1cn2c(-c3cccc(N[C@H]4CNC[C@@H]4F)n3)cnc2cn1] |
| 63-9 | 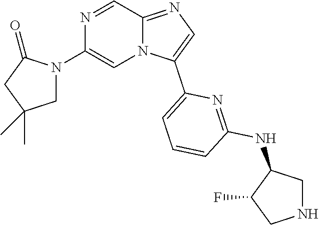 [SMILES: CC1(C)CC(=O)N(c2cn3c(-c4cccc(N[C@H]5CNC[C@@H]5F)n4)cnc3cn2)C1] |
| 64-9 | 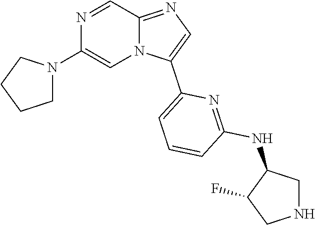 [SMILES: F[C@H]1CNC[C@@H]1Nc1cccc(-c2cnc3cnc(N4CCCC4)cn23)n1] |
| 65-9 | 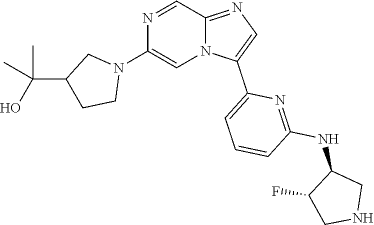 [SMILES: CC(C)(O)C1CCN(c2cn3c(-c4cccc(N[C@H]5CNC[C@@H]5F)n4)cnc3cn2)C1] |
| 66-9 | 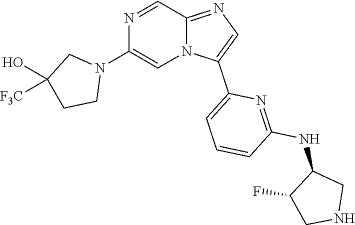 [SMILES: OC1(C(F)(F)F)CCN(c2cn3c(-c4cccc(N[C@H]5CNC[C@@H]5F)n4)cnc3cn2)C1] |
| 67-9 | 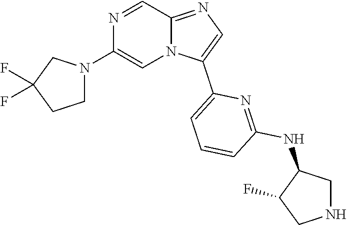 [SMILES: F[C@H]1CNC[C@@H]1Nc1cccc(-c2cnc3cnc(N4CCC(F)(F)C4)cn23)n1] |
| 68-9 | 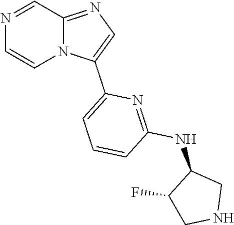 [SMILES: F[C@H]1CNC[C@@H]1Nc1cccc(-c2cnc3cnccn23)n1] |
| 69-9 | 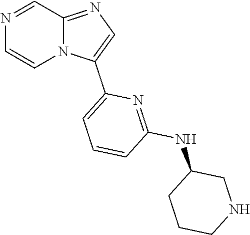 [SMILES: c1cc(N[C@@H]2CCCNC2)nc(-c2cnc3cnccn23)c1] |
| 70-9 | 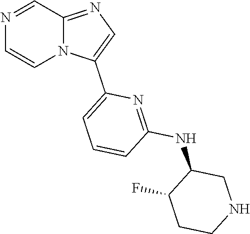 [SMILES: F[C@H]1CCNC[C@@H]1Nc1cccc(-c2cnc3cnccn23)n1] |
| 71-9 | 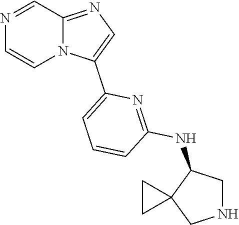 [SMILES: c1cc(N[C@H]2CNCC23CC3)nc(-c2cnc3cnccn23)c1] |
| 72-9 | 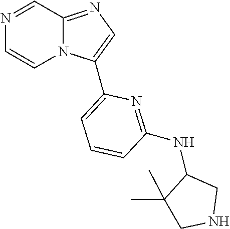 [SMILES: CC1(C)CNCC1Nc1cccc(-c2cnc3cnccn23)n1] |
| 73-9 | 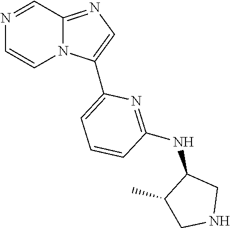 [SMILES: C[C@H]1CNC[C@@H]1Nc1cccc(-c2cnc3cnccn23)n1] |
| 74-9 | 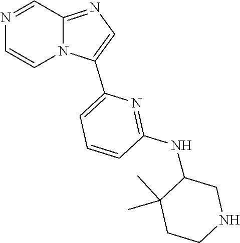 [SMILES: CC1(C)CCNCC1Nc1cccc(-c2cnc3cnccn23)n1] |
| 75-9 | 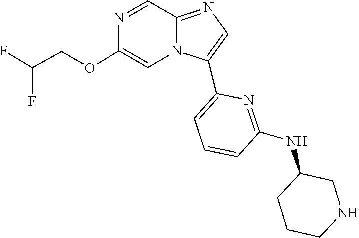 [SMILES: FC(F)COc1cn2c(-c3cccc(N[C@@H]4CCCNC4)n3)cnc2cn1] |
| 76-9 | 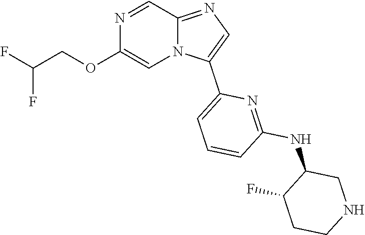 [SMILES: FC(F)COc1cn2c(-c3cccc(N[C@H]4CNCC[C@@H]4F)n3)cnc2cn1] |
| 77-9 | 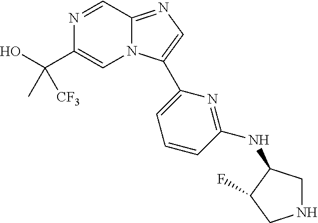 [SMILES: CC(O)(c1cn2c(-c3cccc(N[C@H]4CNC[C@@H]4F)n3)cnc2cn1)C(F)(F)F] |
| 78-9 | 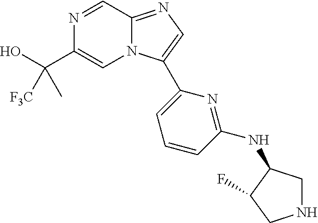 [SMILES: CC(O)(c1cn2c(-c3cccc(N[C@H]4CNC[C@@H]4F)n3)cnc2cn1)C(F)(F)F] |
| 79-9 | 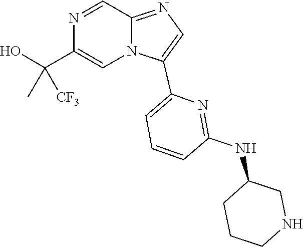 [SMILES: CC(O)(c1cn2c(-c3cccc(N[C@@H]4CCCNC4)n3)cnc2cn1)C(F)(F)F] |
| 80-9 | 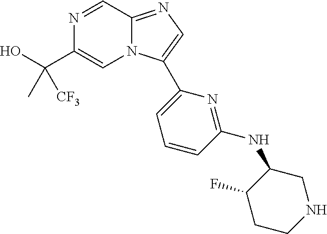 [SMILES: CC(O)(c1cn2c(-c3cccc(N[C@H]4CNCC[C@@H]4F)n3)cnc2cn1)C(F)(F)F] |
| 81-9 | 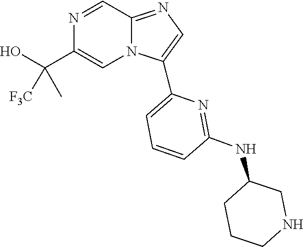 [SMILES: CC(O)(c1cn2c(-c3cccc(N[C@@H]4CCCNC4)n3)cnc2cn1)C(F)(F)F] |
| 82-9 | 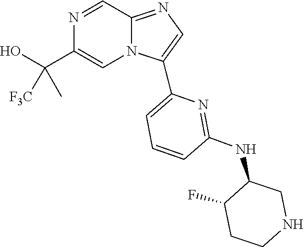 [SMILES: CC(O)(c1cn2c(-c3cccc(N[C@H]4CNCC[C@@H]4F)n3)cnc2cn1)C(F)(F)F] |
| 83-9 | 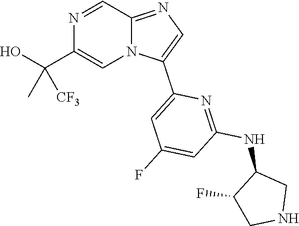 [SMILES: CC(O)(c1cn2c(-c3cc(F)cc(N[C@H]4CNC[C@@H]4F)n3)cnc2cn1)C(F)(F)F] |
| 84-9 | 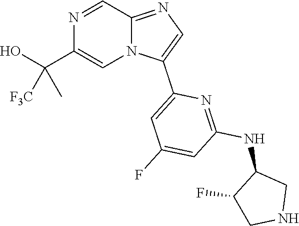 [SMILES: CC(O)(c1cn2c(-c3cc(F)cc(N[C@H]4CNC[C@@H]4F)n3)cnc2cn1)C(F)(F)F] |
| 85-9 | 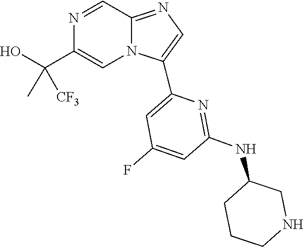 [SMILES: CC(O)(c1cn2c(-c3cc(F)cc(N[C@@H]4CCCNC4)n3)cnc2cn1)C(F)(F)F] |
| 86-9 | 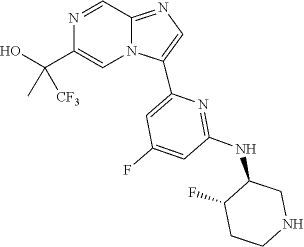 [SMILES: CC(O)(c1cn2c(-c3cc(F)cc(N[C@H]4CNCC[C@@H]4F)n3)cnc2cn1)C(F)(F)F] |
| 87-9 | 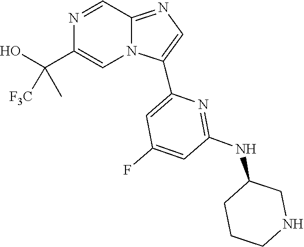 [SMILES: CC(O)(c1cn2c(-c3cc(F)cc(N[C@@H]4CCCNC4)n3)cnc2cn1)C(F)(F)F] |
| 88-9 | 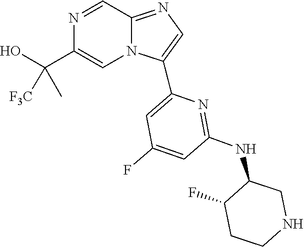 [SMILES: CC(O)(c1cn2c(-c3cc(F)cc(N[C@H]4CNCC[C@@H]4F)n3)cnc2cn1)C(F)(F)F] |
| 89-9 | 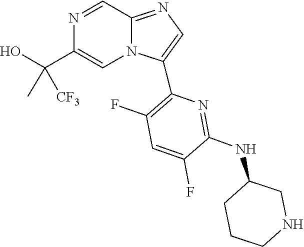 [SMILES: CC(O)(c1cn2c(-c3nc(N[C@@H]4CCCNC4)c(F)cc3F)cnc2cn1)C(F)(F)F] |
| 90-9 | 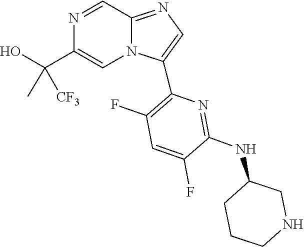 [SMILES: CC(O)(c1cn2c(-c3nc(N[C@@H]4CCCNC4)c(F)cc3F)cnc2cn1)C(F)(F)F] |
| 91-9 | 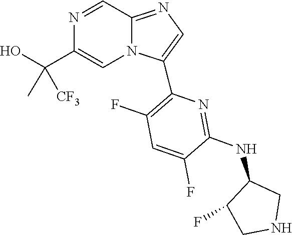 [SMILES: CC(O)(c1cn2c(-c3nc(N[C@H]4CNC[C@@H]4F)c(F)cc3F)cnc2cn1)C(F)(F)F] |
| 92-9 | 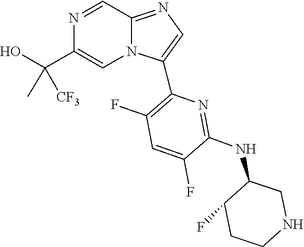 [SMILES: CC(O)(c1cn2c(-c3nc(N[C@H]4CNCC[C@@H]4F)c(F)cc3F)cnc2cn1)C(F)(F)F] |
| 93-9 | 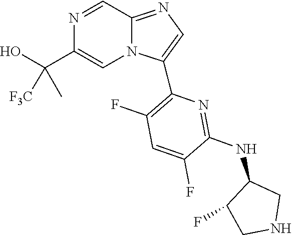 [SMILES: CC(O)(c1cn2c(-c3nc(N[C@H]4CNC[C@@H]4F)c(F)cc3F)cnc2cn1)C(F)(F)F] |
| 94-9 | 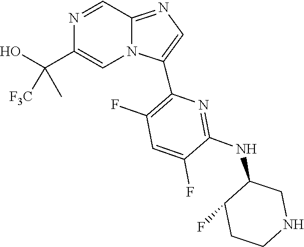 [SMILES: CC(O)(c1cn2c(-c3nc(N[C@H]4CNCC[C@@H]4F)c(F)cc3F)cnc2cn1)C(F)(F)F] |
| 95-9 | 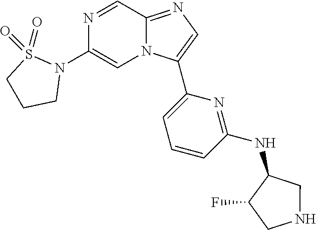 [SMILES: O=S1(=O)CCCN1c1cn2c(-c3cccc(N[C@H]4CNC[C@@H]4F)n3)cnc2cn1] |
| 96-9 | 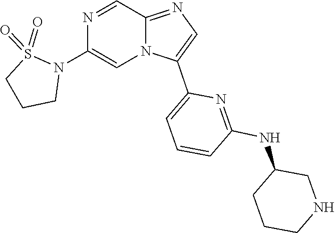 [SMILES: O=S1(=O)CCCN1c1cn2c(-c3cccc(N[C@@H]4CCCNC4)n3)cnc2cn1] |
| 97-9 | 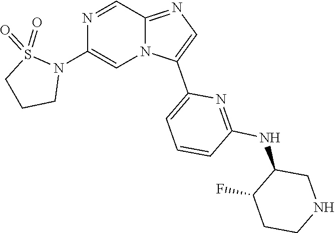 [SMILES: O=S1(=O)CCCN1c1cn2c(-c3cccc(N[C@H]4CNCC[C@@H]4F)n3)cnc2cn1] |
| 98-9 | 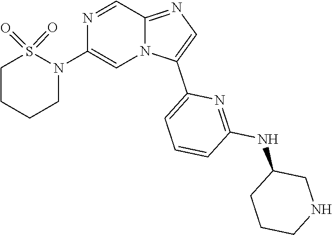 [SMILES: O=S1(=O)CCCCN1c1cn2c(-c3cccc(N[C@@H]4CCCNC4)n3)cnc2cn1] |
| 99-9 | 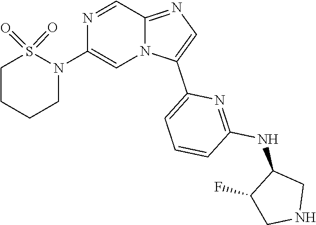 [SMILES: O=S1(=O)CCCCN1c1cn2c(-c3cccc(N[C@H]4CNC[C@@H]4F)n3)cnc2cn1] |
| 100-9 | 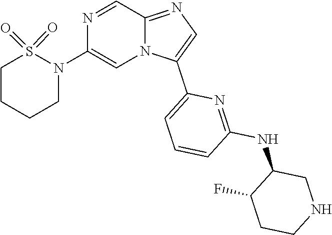 [SMILES: O=S1(=O)CCCCN1c1cn2c(-c3cccc(N[C@H]4CNCC[C@@H]4F)n3)cnc2cn1] |
| 101-9 | 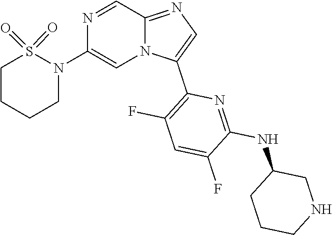 [SMILES: O=S1(=O)CCCCN1c1cn2c(-c3nc(N[C@@H]4CCCNC4)c(F)cc3F)cnc2cn1] |
| 102-9 | 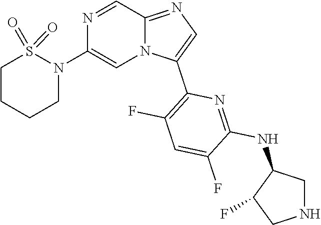 [SMILES: O=S1(=O)CCCCN1c1cn2c(-c3nc(N[C@H]4CNC[C@@H]4F)c(F)cc3F)cnc2cn1] |
| 103-9 | 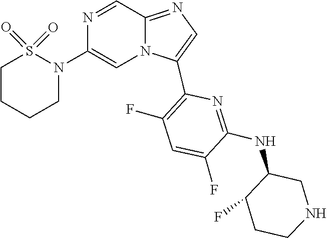 [SMILES: O=S1(=O)CCCCN1c1cn2c(-c3nc(N[C@H]4CNCC[C@@H]4F)c(F)cc3F)cnc2cn1] |
| 104-9 | 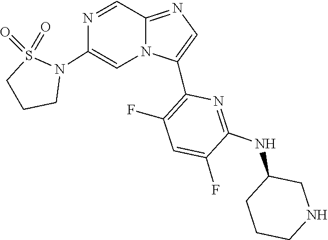 [SMILES: O=S1(=O)CCCN1c1cn2c(-c3nc(N[C@@H]4CCCNC4)c(F)cc3F)cnc2cn1] |
| 105-9 | 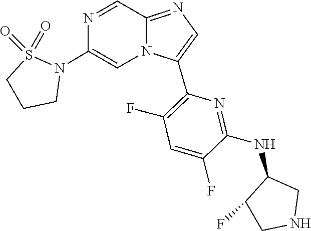 [SMILES: O=S1(=O)CCCN1c1cn2c(-c3nc(N[C@H]4CNC[C@@H]4F)c(F)cc3F)cnc2cn1] |
| 106-9 | 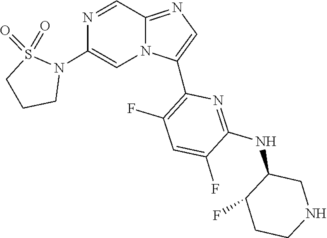 [SMILES: O=S1(=O)CCCN1c1cn2c(-c3nc(N[C@H]4CNCC[C@@H]4F)c(F)cc3F)cnc2cn1] |
| 107-9 | 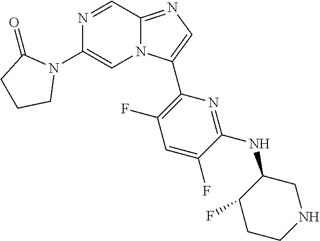 [SMILES: O=C1CCCN1c1cn2c(-c3nc(N[C@H]4CNCC[C@@H]4F)c(F)cc3F)cnc2cn1] |
| 108-9 | 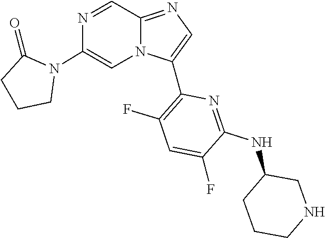 [SMILES: O=C1CCCN1c1cn2c(-c3nc(N[C@@H]4CCCNC4)c(F)cc3F)cnc2cn1] |
| 109-9 | 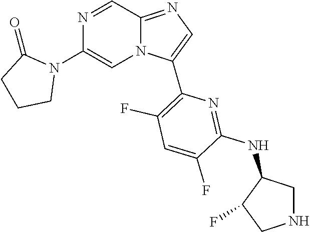 [SMILES: O=C1CCCN1c1cn2c(-c3nc(N[C@H]4CNC[C@@H]4F)c(F)cc3F)cnc2cn1] |
| 110-9 | 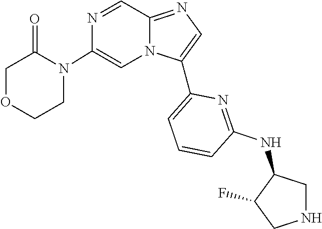 [SMILES: O=C1COCCN1c1cn2c(-c3cccc(N[C@H]4CNC[C@@H]4F)n3)cnc2cn1] |
| 111-9 | 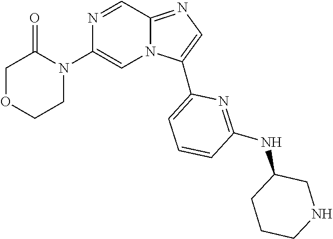 [SMILES: O=C1COCCN1c1cn2c(-c3cccc(N[C@@H]4CCCNC4)n3)cnc2cn1] |
| 112-9 | 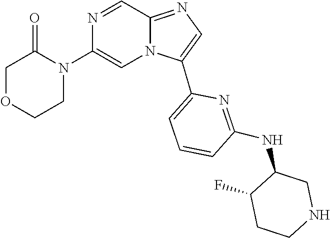 [SMILES: O=C1COCCN1c1cn2c(-c3cccc(N[C@H]4CNCC[C@@H]4F)n3)cnc2cn1] |
| 113-9 | 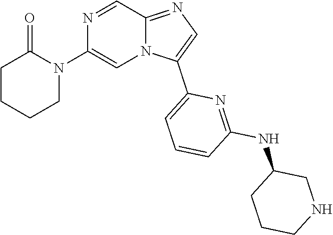 [SMILES: O=C1CCCCN1c1cn2c(-c3cccc(N[C@@H]4CCCNC4)n3)cnc2cn1] |
| 114-9 | 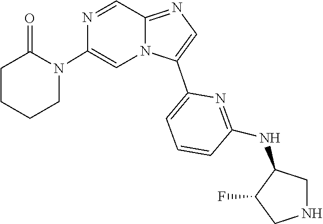 [SMILES: O=C1CCCCN1c1cn2c(-c3cccc(N[C@H]4CNC[C@@H]4F)n3)cnc2cn1] |
| 115-9 | 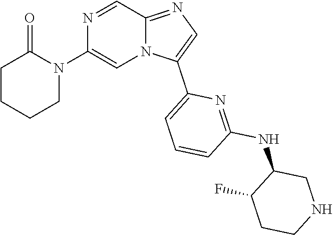 [SMILES: O=C1CCCCN1c1cn2c(-c3cccc(N[C@H]4CNCC[C@@H]4F)n3)cnc2cn1] |
| 116-9 | 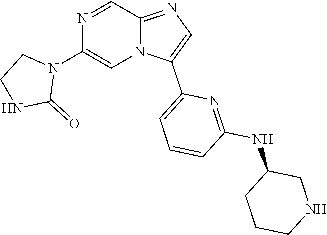 [SMILES: O=C1NCCN1c1cn2c(-c3cccc(N[C@@H]4CCCNC4)n3)cnc2cn1] |
| 117-9 | 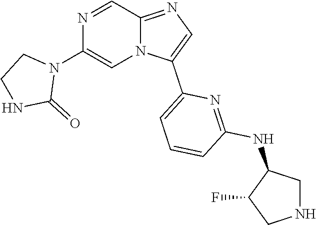 [SMILES: O=C1NCCN1c1cn2c(-c3cccc(N[C@H]4CNC[C@@H]4F)n3)cnc2cn1] |
| 118-9 | 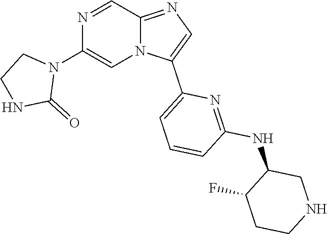 [SMILES: O=C1NCCN1c1cn2c(-c3cccc(N[C@H]4CNCC[C@@H]4F)n3)cnc2cn1] |
| 119-9 | 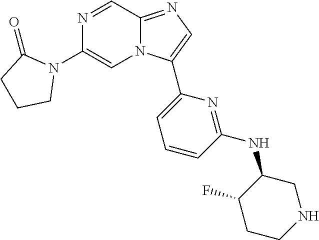 [SMILES: O=C1CCCN1c1cn2c(-c3cccc(N[C@H]4CNCC[C@@H]4F)n3)cnc2cn1] |
| 120-9 | 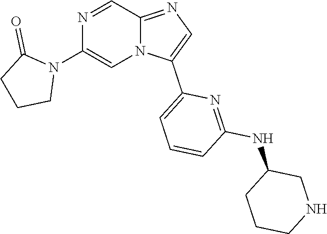 [SMILES: O=C1CCCN1c1cn2c(-c3cccc(N[C@@H]4CCCNC4)n3)cnc2cn1] |
| 121-9 | 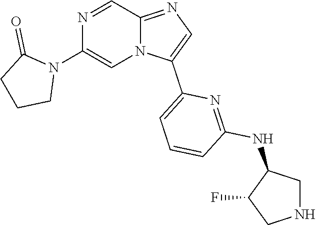 [SMILES: O=C1CCCN1c1cn2c(-c3cccc(N[C@H]4CNC[C@@H]4F)n3)cnc2cn1] |
| 122-9 | 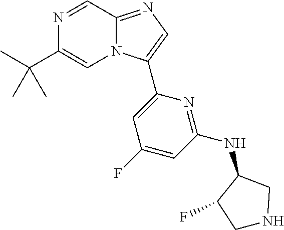 [SMILES: CC(C)(C)c1cn2c(-c3cc(F)cc(N[C@H]4CNC[C@@H]4F)n3)cnc2cn1] |
| 123-9 | 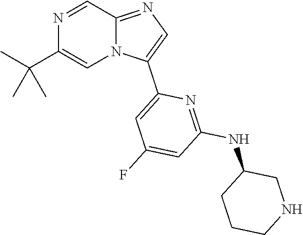 [SMILES: CC(C)(C)c1cn2c(-c3cc(F)cc(N[C@@H]4CCCNC4)n3)cnc2cn1] |
| 124-9 | 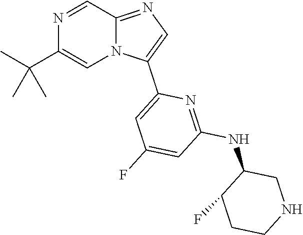 [SMILES: CC(C)(C)c1cn2c(-c3cc(F)cc(N[C@H]4CNCC[C@@H]4F)n3)cnc2cn1] |
| 125-9 | 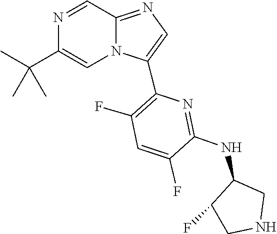 [SMILES: CC(C)(C)c1cn2c(-c3nc(N[C@H]4CNC[C@@H]4F)c(F)cc3F)cnc2cn1] |
| 126-9 | 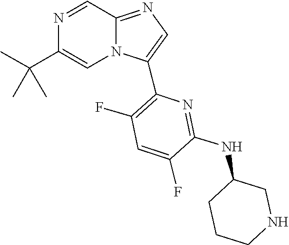 [SMILES: CC(C)(C)c1cn2c(-c3nc(N[C@@H]4CCCNC4)c(F)cc3F)cnc2cn1] |
| 127-9 | 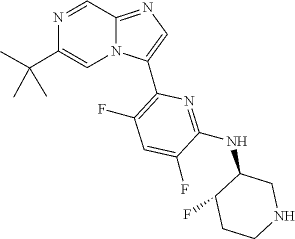 [SMILES: CC(C)(C)c1cn2c(-c3nc(N[C@H]4CNCC[C@@H]4F)c(F)cc3F)cnc2cn1] |
| 128-9 | 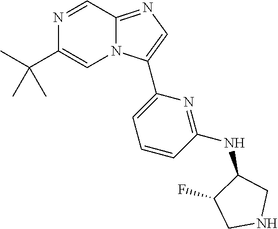 [SMILES: CC(C)(C)c1cn2c(-c3cccc(N[C@H]4CNC[C@@H]4F)n3)cnc2cn1] |
| 129-9 | 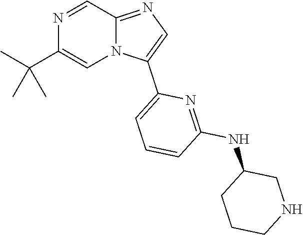 [SMILES: CC(C)(C)c1cn2c(-c3cccc(N[C@@H]4CCCNC4)n3)cnc2cn1] |
| 130-9 | 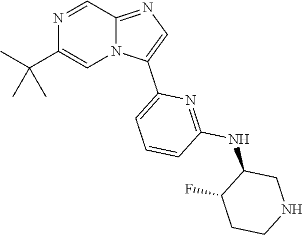 [SMILES: CC(C)(C)c1cn2c(-c3cccc(N[C@H]4CNCC[C@@H]4F)n3)cnc2cn1] |
| 131-9 | 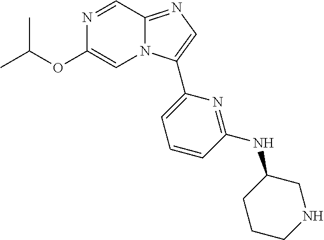 [SMILES: CC(C)Oc1cn2c(-c3cccc(N[C@@H]4CCCNC4)n3)cnc2cn1] |
| 132-9 | 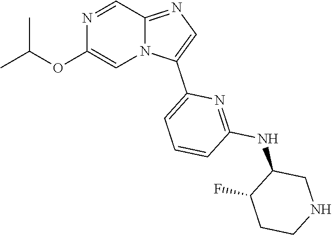 [SMILES: CC(C)Oc1cn2c(-c3cccc(N[C@H]4CNCC[C@@H]4F)n3)cnc2cn1] |
| 133-9 | 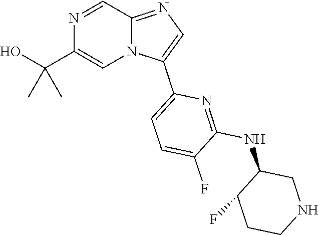 [SMILES: CC(C)(O)c1cn2c(-c3ccc(F)c(N[C@H]4CNCC[C@@H]4F)n3)cnc2cn1] |
| 134-9 | 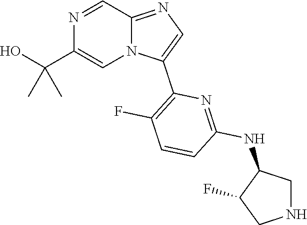 [SMILES: CC(C)(O)c1cn2c(-c3nc(N[C@H]4CNC[C@@H]4F)ccc3F)cnc2cn1] |
| 135-9 | 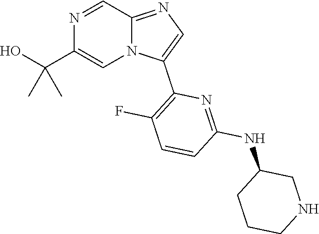 [SMILES: CC(C)(O)c1cn2c(-c3nc(N[C@@H]4CCCNC4)ccc3F)cnc2cn1] |
| 136-9 | 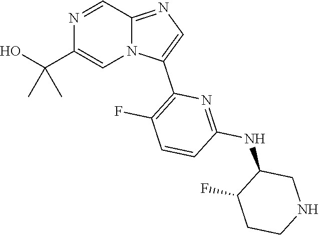 [SMILES: CC(C)(O)c1cn2c(-c3nc(N[C@H]4CNCC[C@@H]4F)ccc3F)cnc2cn1] |
| 137-9 | 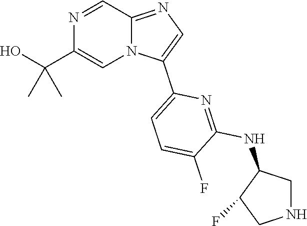 [SMILES: CC(C)(O)c1cn2c(-c3ccc(F)c(N[C@H]4CNC[C@@H]4F)n3)cnc2cn1] |
| 138-9 | 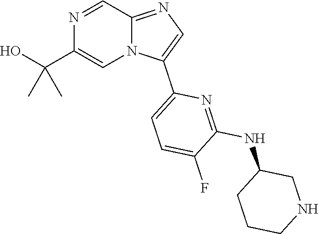 [SMILES: CC(C)(O)c1cn2c(-c3ccc(F)c(N[C@@H]4CCCNC4)n3)cnc2cn1] |

In one embodiment, the compound is a compound of Formula (I-5010), (II-5010), or (III-5010):

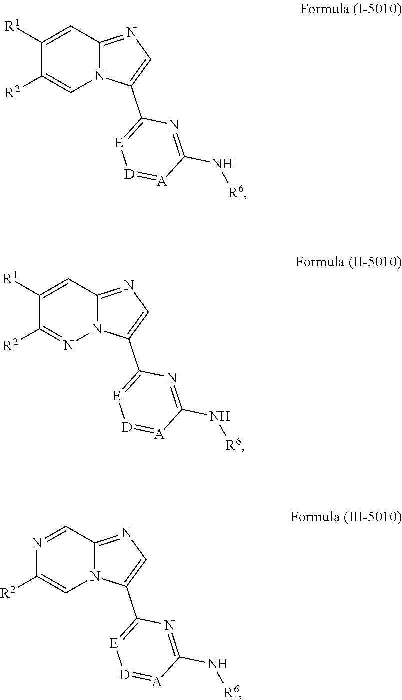

or a salt, ester, solvate, optical isomer, geometric isomer, or salt of an isomer thereof; wherein:
A is selected from N and CR⁵;
D is selected from N and CR⁴;
E is selected from N and CR³;
at least one of A, D, and E is N;
R₁, R₂, R₃, R₄, and R₅ are each independently selected from H, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, —C(═O)NR₃₁R₃₂, cycloalkyl, spiro-fused cycloalkyl, heterocyclyl, —O-aryl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, —O-aryl, aryl, heteroaryl, or fused ring heteroaryl is optionally substituted with one or more of halogen, hydroxy, oxo, —C(═O)H, —C(═O)OH, nitro (—NO₂), —NH₂, —N(CH₃)₂, cyano (—CN), ethynyl (—CCH), propynyl, —SO₃H, heterocyclyl, aryl, heteroaryl, pyrrolyl, piperidyl, piperazinyl, morpholinyl, —C(═O)-morpholin-4-yl, —C(═O)NH₂, —C(═O)N(CH₃)₂, C₁-C₇ alkyl, C₁-C₇ perfluorinated alkyl, C₁-C₇ alkoxy, C₁-C₇ haloalkoxy, or C₁-C₇ alkyl which is substituted with cycloalkyl;
R⁶ is

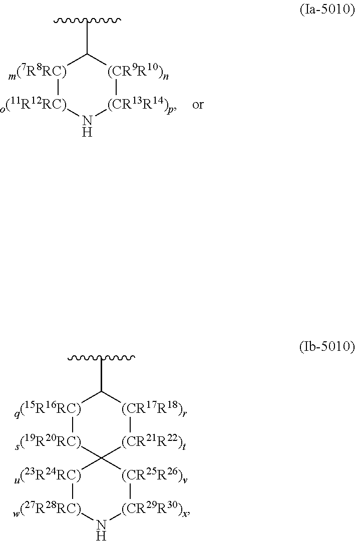

or C₃-C₆ cycloalkyl substituted with one or more —NR₃₃R³⁴;
R⁷, R⁸, R⁹, R¹⁰, R, R¹², R¹³, and R¹⁴ are each independently selected from H, halogen, hydroxy, oxo, —CN, —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen;
R¹⁵, R¹⁶, R′⁷, R¹⁸, R¹⁹, R²⁰, R²¹, R²², R²³, R²⁴, R²⁵, R²⁶, R²⁷, R²⁸, R²⁹, and R³⁰ are each independently selected from H, halogen, hydroxy, oxo, —CN, methanoyl (—COH), carboxy (—CO₂H), C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl, wherein —C(═O)H, —C(═O)OH, C₁-C₇ alkyl, C₂-C₇ alkenyl, C₂-C₇ alkynyl, C₁-C₇ alkoxy, cycloalkyl, spiro-fused cycloalkyl, heterocyclyl, aryl, heteroaryl, or fused ring heteroaryl is optionally substituted with one or more halogen;
R³¹ and R³² are each independently selected from H, C₁-C₆ alkyl, and C₃-C₆ cycloalkyl, wherein C₁-C₆ alkyl and C₃-C₆ cycloalkyl are optionally substituted with one or more halogen;
R³³ and R³⁴ are each independently selected from H and C₁-C₆ alkyl; and
m, n, o, p, q, r, s, t, u, v, w, and x are independently selected from 0, 1, 2, 3, 4, or 5, where q+r+s+t is at least 1, and where u+v+w+x is at least 1.

In one embodiment, the compound of Formula (1-5010) is a compound of Formula (Ia-5010):

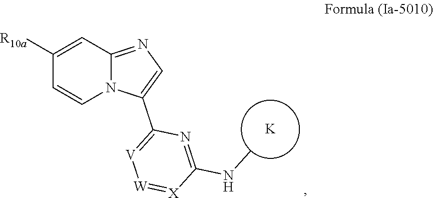

or a salt, ester, solvate, optical isomer, geometric isomer, or salt of an isomer thereof; wherein:
V is N or CR₁₁;
W is N or CR₁₂;
X is N or CR₁₃;

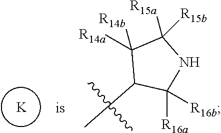

R₁₀ₐ is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In one embodiment, the compound of Formula (1-5010) is a compound of Formula (Ib-5010):

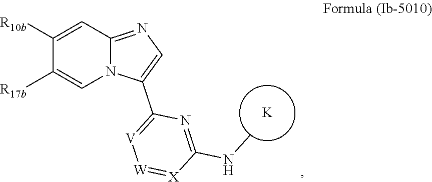

or a salt, ester, solvate, optical isomer, geometric isomer, or salt of an isomer thereof; wherein:
V is N or CR₁₁;
W is N or CR₁₂;
X is N or CR₁₃;

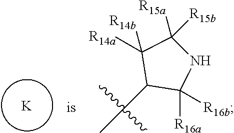

R_{10b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{17b} is selected from halogen, C₁-C₆ alkyl, C₁-C₆ alkoxy, C₃-C₆ cycloalkyl, —O—(C₃-C₆ cycloalkyl), C₃-C₉ heterocyclyl, imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₄ₐ, R_{14b}, R₁₅ₐ, R_{15b}, R₁₆ₐ, R_{16b}, R₁₈ₐ, and R_{18b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In one embodiment, the compound of Formula (1-5010) is a compound of Formula (Ic-5010):

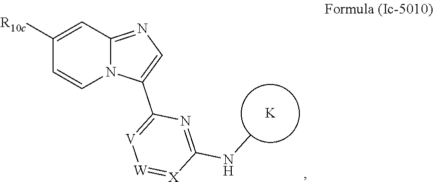

or a salt, ester, solvate, optical isomer, geometric isomer, or salt of an isomer thereof; wherein:
V is N or CR₁₁;
W is N or CR₁₂;
X is N or CR₁₃;

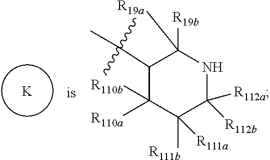

R_{10c} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₈ₐ, R_{18b}, R₁₉ₐ, R_{19b}, R₁₁₀ₐ, R_{110b}, R₁₁₁ₐ, R_{111b}, R_{112az}, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and one of V, W, or X is N.

In one embodiment, the compound of Formula (1-5010) is a compound of Formula (Id-5010):

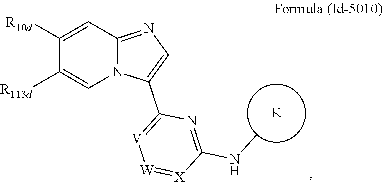

or a salt, ester, solvate, optical isomer, geometric isomer, or salt of an isomer thereof; wherein:
V is N or CR₁₁;
W is N or CR₁₂;
X is N or CR₁₃;

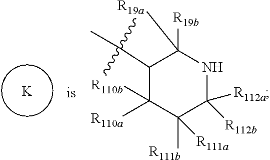

R_{10d} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{113d} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₁₈ₐR_{18b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₁₁, R₁₂, and R₁₃ are each independently selected from H and halogen;
R₁₈ₐ, R_{18b}, R₁₉ₐ, R_{19b}, R₁₁₀ₐ, R_{110b}, R₁₁₁ₐ, R_{111b}, R₁₁₂ₐ, and R_{112b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of V, W, or X is N.

In one embodiment, the compound of Formula (II-5010) is a compound of Formula (IIa-5010):

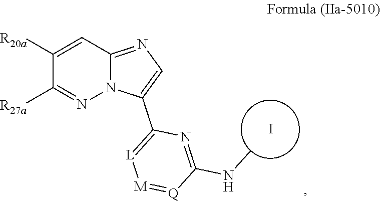

or a salt, ester, solvate, optical isomer, geometric isomer, or salt of an isomer thereof; wherein:
L is N or CR₂₁;
M is N or CR₂₂;
Q is N or CR₂₃;

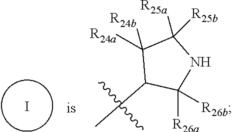

R₂₀ₐ is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₇ₐ is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₁, R₂₂, and R₂₃ are each independently selected from H and halogen;
R₂₄ₐ, R_{24b}, R₂₅ₐ, R_{25b}, R₂₆ₐ, R_{26b}, R₂₉ₐ, and R_{28b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of L, M, or Q is N.

In one embodiment, the compound of Formula (II-5010) is a compound of Formula (IIb-5010):

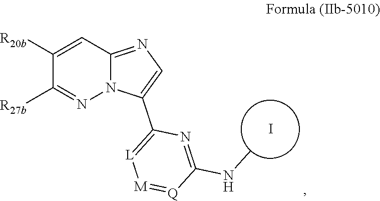

or a salt, ester, solvate, optical isomer, geometric isomer, or salt of an isomer thereof, wherein:
L is N or CR₂₁;
M is N or CR₂₂;
Q is N or CR₂₃;

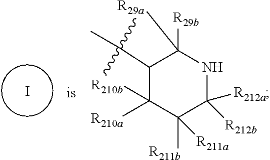

R_{20b} is selected from H, halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R_{27b} is selected from halogen, C₁-C₆ alkyl, C₁-C₆ alkoxy, C₃-C₆ cycloalkyl, —O—(C₃-C₆ cycloalkyl), imidazolyl, triazolyl, and —C(═O)NR₂₈ₐR_{28b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₂₁, R₂₂, and R₂₃ are each independently selected from H and halogen;
R₂₉ₐ, R_{29b}, R₂₁₀ₐ, R_{210b}, R₂₁₁ₐ, R_{211b}, R₂₁₂ₐ, and R_{212b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of L, M, or Q is N.

In one embodiment, the compound of Formula (III-5010) is a compound of Formula (IIIa-5010):

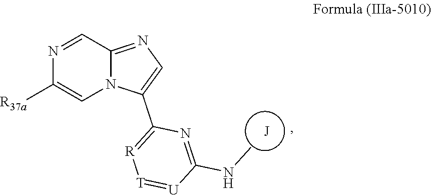

or a salt, ester, solvate, optical isomer, geometric isomer, or salt of an isomer thereof;
wherein:
R is N or CR₃₁;
T is N or CR₃₂,
U is N or CR₃₃;

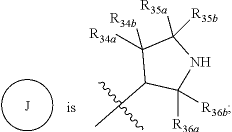

R₃₇ₐ is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₃₁, R₃₂, and R₃₃ are each independently selected from H and halogen;
R₃₄ₐ, R_{34b}, R₃₅ₐ, R_{35b}, R₃₆ₐ, R_{36b}, R₃₉ₐ, and R_{3sb} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of R, T, or U is N.

In one embodiment, the compound of Formula (III-5010) is a compound of Formula (IIIb-5010):

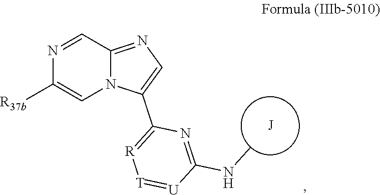

or a salt, ester, solvate, optical isomer, geometric isomer, or salt of an isomer thereof;
wherein:
R is N or CR₃₁;
T is Nor CR₃₂,
U is N or CR₃₃;

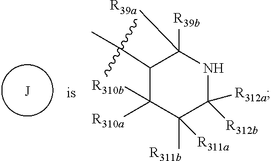

R_{37b} is selected from halogen, C₁-C₆ alkyl, C₁-C₆ alkoxy, C₃-C₆ cycloalkyl, —O—(C₃-C₆ cycloalkyl), imidazolyl, triazolyl, 2-pyrrolidinonyl, and —C(═O)NR₃₈ₐR_{38b}, wherein C₁-C₆ alkyl and C₁-C₆ alkoxy are each optionally substituted with one or more substituents selected from —OH and halogen, and C₃-C₆ cycloalkyl and —O—(C₃-C₆ cycloalkyl) are each optionally substituted with one or more substituents selected from C₁-C₆ alkyl and halogen;
R₃₁, R₃₂, and R₃₃ are each independently selected from H and halogen;
R₃₈ₐ, R_{38b}, R₃₉ₐ, R_{39b}, R₃₁₀ₐ, R_{310b}, R₃₁₁ₐ, R_{311b}, R₃₁₂ₐ, and R_{312b} are each independently selected from H, halogen, —OH, C₁-C₆ alkyl, and C₁-C₆ alkoxy, wherein C₁-C₆ alkyl and C₁-C₆ alkoxy are each optionally substituted with one or more halogen atoms; and
one of R, T, or U is N.

In one embodiment, the compound of Formula (1-5010), (II-5010), or (III-5010) is selected from Compounds 1-10 to 84-10.

| # | Structure |
|---|---|
| 1-10 | 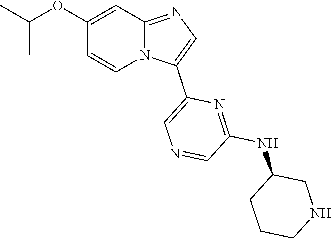 [SMILES: CC(C)Oc1ccn2c(-c3cncc(N[C@@H]4CCCNC4)n3)cnc2c1] |
| 2-10 | 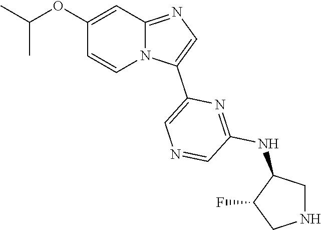 [SMILES: CC(C)Oc1ccn2c(-c3cncc(N[C@H]4CNC[C@@H]4F)n3)cnc2c1] |
| 3-10 | 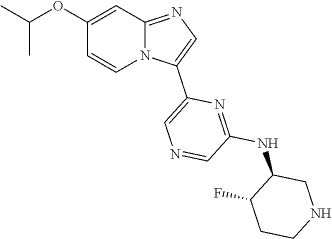 [SMILES: CC(C)Oc1ccn2c(-c3cncc(N[C@H]4CNCC[C@@H]4F)n3)cnc2c1] |
| 4-10 | 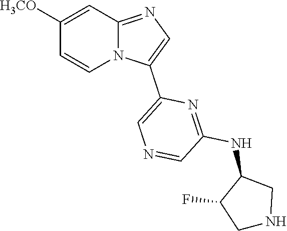 [SMILES: COc1ccn2c(-c3cncc(N[C@H]4CNC[C@@H]4F)n3)cnc2c1] |
| 5-10 | 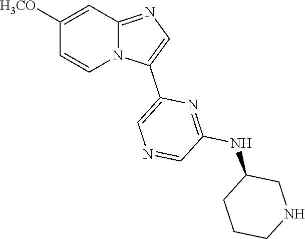 [SMILES: COc1ccn2c(-c3cncc(N[C@@H]4CCCNC4)n3)cnc2c1] |
| 6-10 | 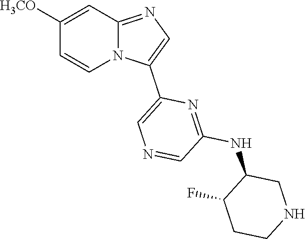 [SMILES: COc1ccn2c(-c3cncc(N[C@H]4CNCC[C@@H]4F)n3)cnc2c1] |
| 7-10 | 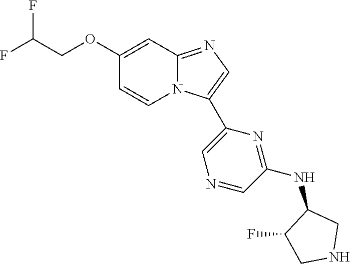 [SMILES: FC(F)COc1ccn2c(-c3cncc(N[C@H]4CNC[C@@H]4F)n3)cnc2c1] |
| 8-10 | 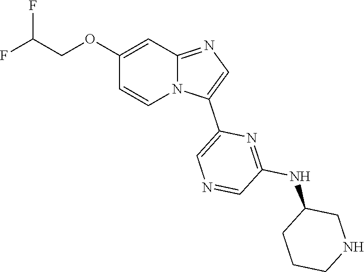 [SMILES: FC(F)COc1ccn2c(-c3cncc(N[C@@H]4CCCNC4)n3)cnc2c1] |
| 9-10 | 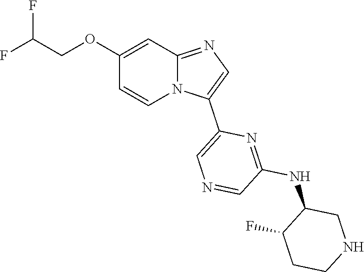 [SMILES: FC(F)COc1ccn2c(-c3cncc(N[C@H]4CNCC[C@@H]4F)n3)cnc2c1] |
| 10-10 | 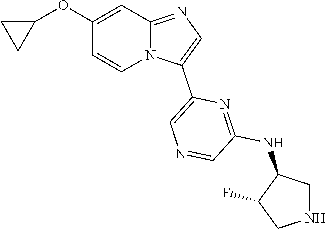 [SMILES: F[C@H]1CNC[C@@H]1Nc1cncc(-c2cnc3cc(OC4CC4)ccn23)n1] |
| 11-10 | 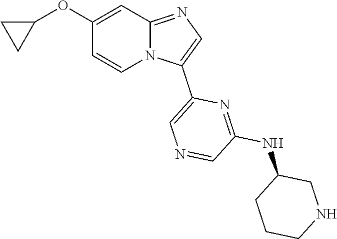 [SMILES: c1cn2c(-c3cncc(N[C@@H]4CCCNC4)n3)cnc2cc1OC1CC1] |
| 12-10 | 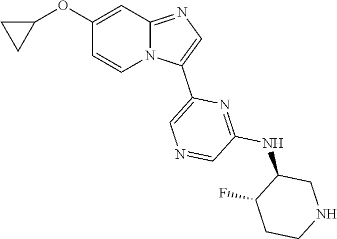 [SMILES: F[C@H]1CCNC[C@@H]1Nc1cncc(-c2cnc3cc(OC4CC4)ccn23)n1] |
| 13-10 | 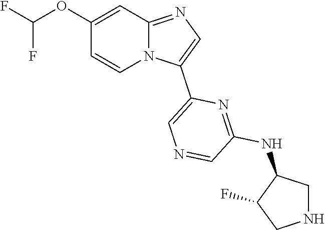 [SMILES: FC(F)Oc1ccn2c(-c3cncc(N[C@H]4CNC[C@@H]4F)n3)cnc2c1] |
| 14-10 | 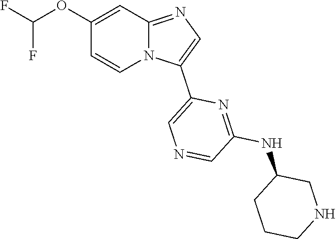 [SMILES: FC(F)Oc1ccn2c(-c3cncc(N[C@@H]4CCCNC4)n3)cnc2c1] |
| 15-10 | 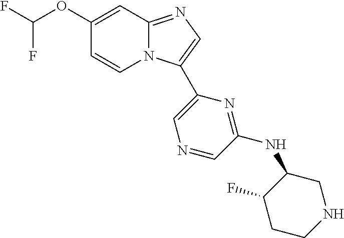 [SMILES: FC(F)Oc1ccn2c(-c3cncc(N[C@H]4CNCC[C@@H]4F)n3)cnc2c1] |
| 16-10 | 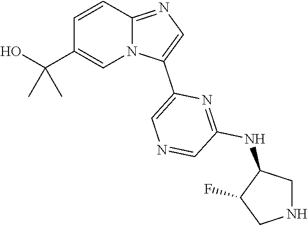 [SMILES: CC(C)(O)c1ccc2ncc(-c3cncc(N[C@H]4CNC[C@@H]4F)n3)n2c1] |
| 17-10 | 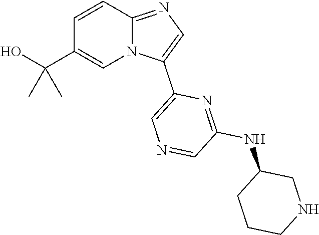 [SMILES: CC(C)(O)c1ccc2ncc(-c3cncc(N[C@@H]4CCCNC4)n3)n2c1] |
| 18-10 | 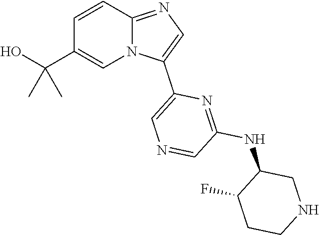 [SMILES: CC(C)(O)c1ccc2ncc(-c3cncc(N[C@H]4CNCC[C@@H]4F)n3)n2c1] |
| 19-10 | 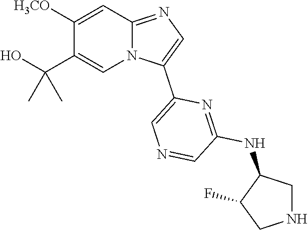 [SMILES: COc1cc2ncc(-c3cncc(N[C@H]4CNC[C@@H]4F)n3)n2cc1C(C)(C)O] |
| 20-10 | 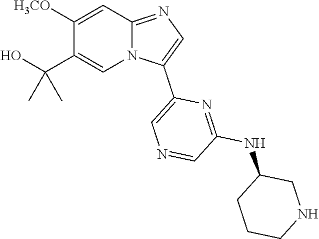 [SMILES: COc1cc2ncc(-c3cncc(N[C@@H]4CCCNC4)n3)n2cc1C(C)(C)O] |
| 21-10 | 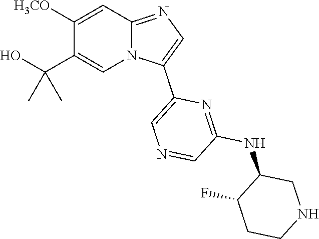 [SMILES: COc1cc2ncc(-c3cncc(N[C@H]4CNCC[C@@H]4F)n3)n2cc1C(C)(C)O] |
| 22-10 | 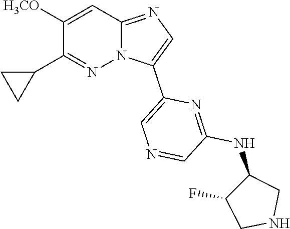 [SMILES: COc1cc2ncc(-c3cncc(N[C@H]4CNC[C@@H]4F)n3)n2nc1C1CC1] |
| 23-10 | 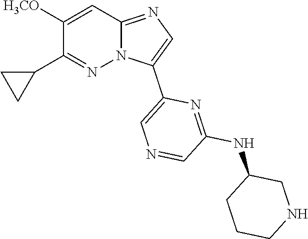 [SMILES: COc1cc2ncc(-c3cncc(N[C@@H]4CCCNC4)n3)n2nc1C1CC1] |
| 24-10 | 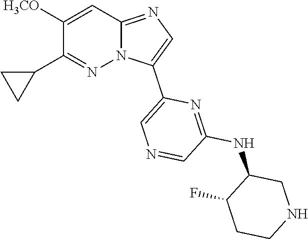 [SMILES: COc1cc2ncc(-c3cncc(N[C@H]4CNCC[C@@H]4F)n3)n2nc1C1CC1] |
| 25-10 | 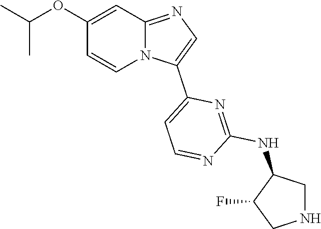 [SMILES: CC(C)Oc1ccn2c(-c3ccnc(N[C@H]4CNC[C@@H]4F)n3)cnc2c1] |
| 26-10 | 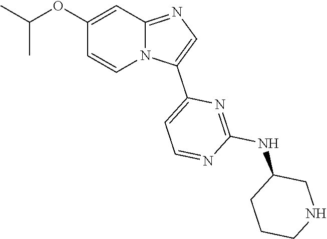 [SMILES: CC(C)Oc1ccn2c(-c3ccnc(N[C@@H]4CCCNC4)n3)cnc2c1] |
| 27-10 | 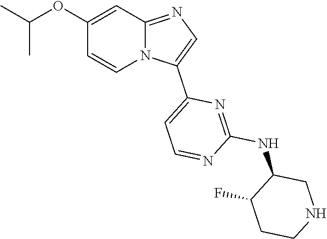 [SMILES: CC(C)Oc1ccn2c(-c3ccnc(N[C@H]4CNCC[C@@H]4F)n3)cnc2c1] |
| 28-10 | 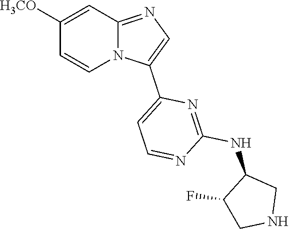 [SMILES: COc1ccn2c(-c3ccnc(N[C@H]4CNC[C@@H]4F)n3)cnc2c1] |
| 29-10 | 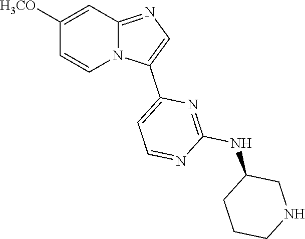 [SMILES: COc1ccn2c(-c3ccnc(N[C@@H]4CCCNC4)n3)cnc2c1] |
| 30-10 | 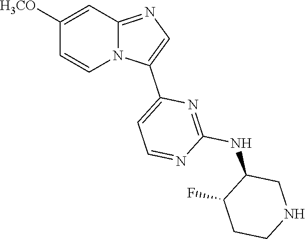 [SMILES: COc1ccn2c(-c3ccnc(N[C@H]4CNCC[C@@H]4F)n3)cnc2c1] |
| 31-10 | 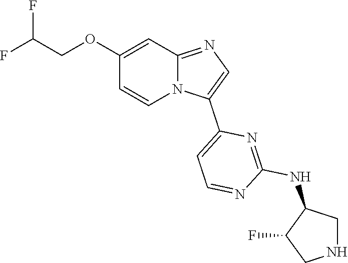 [SMILES: FC(F)COc1ccn2c(-c3ccnc(N[C@H]4CNC[C@@H]4F)n3)cnc2c1] |
| 32-10 | 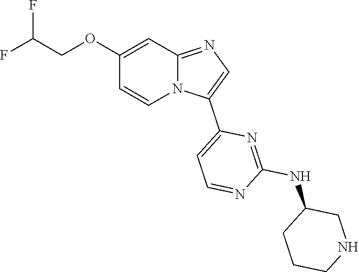 [SMILES: FC(F)COc1ccn2c(-c3ccnc(N[C@@H]4CCCNC4)n3)cnc2c1] |
| 33-10 | 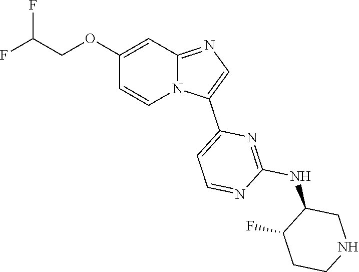 [SMILES: FC(F)COc1ccn2c(-c3ccnc(N[C@H]4CNCC[C@@H]4F)n3)cnc2c1] |
| 34-10 | 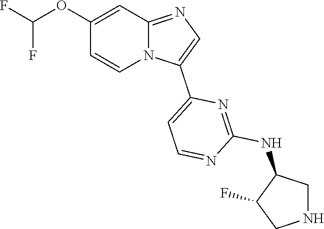 [SMILES: FC(F)Oc1ccn2c(-c3ccnc(N[C@H]4CNC[C@@H]4F)n3)cnc2c1] |
| 35-10 | 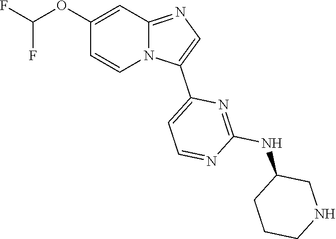 [SMILES: FC(F)Oc1ccn2c(-c3ccnc(N[C@@H]4CCCNC4)n3)cnc2c1] |
| 36-10 | 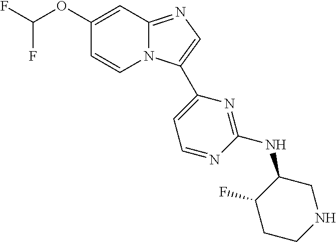 [SMILES: FC(F)Oc1ccn2c(-c3ccnc(N[C@H]4CNCC[C@@H]4F)n3)cnc2c1] |
| 37-10 | 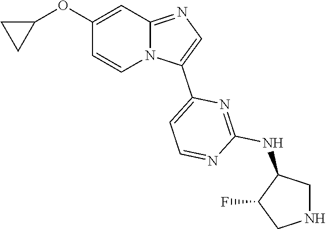 [SMILES: F[C@H]1CNC[C@@H]1Nc1nccc(-c2cnc3cc(OC4CC4)ccn23)n1] |
| 38-10 | 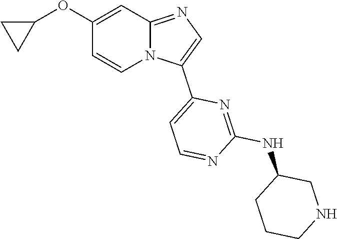 [SMILES: c1cc(-c2cnc3cc(OC4CC4)ccn23)nc(N[C@@H]2CCCNC2)n1] |
| 39-10 | 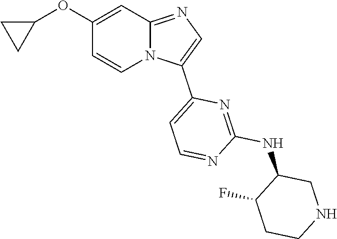 [SMILES: F[C@H]1CCNC[C@@H]1Nc1nccc(-c2cnc3cc(OC4CC4)ccn23)n1] |
| 40-10 | 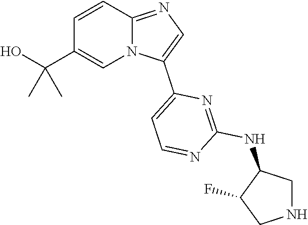 [SMILES: CC(C)(O)c1ccc2ncc(-c3ccnc(N[C@H]4CNC[C@@H]4F)n3)n2c1] |
| 41-10 | 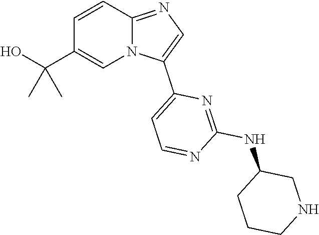 [SMILES: CC(C)(O)c1ccc2ncc(-c3ccnc(N[C@@H]4CCCNC4)n3)n2c1] |
| 42-10 | 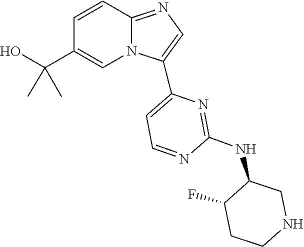 [SMILES: CC(C)(O)c1ccc2ncc(-c3ccnc(N[C@H]4CNCC[C@@H]4F)n3)n2c1] |
| 43-10 | 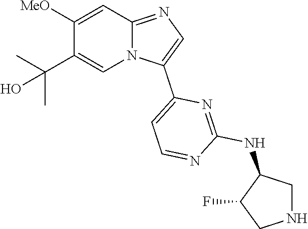 [SMILES: COc1cc2ncc(-c3ccnc(N[C@H]4CNC[C@@H]4F)n3)n2cc1C(C)(C)O] |
| 44-10 | 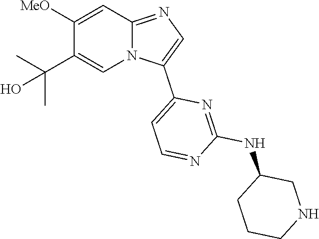 [SMILES: COc1cc2ncc(-c3ccnc(N[C@@H]4CCCNC4)n3)n2cc1C(C)(C)O] |
| 45-10 | 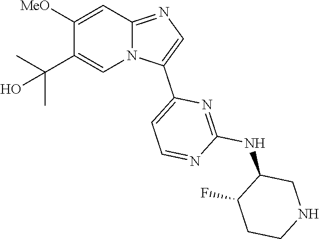 [SMILES: COc1cc2ncc(-c3ccnc(N[C@H]4CNCC[C@@H]4F)n3)n2cc1C(C)(C)O] |
| 46-10 | 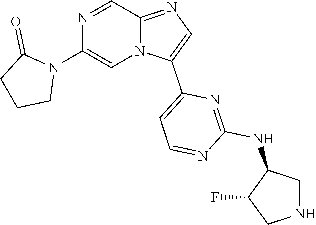 [SMILES: O=C1CCCN1c1cn2c(-c3ccnc(N[C@H]4CNC[C@@H]4F)n3)cnc2cn1] |
| 47-10 | 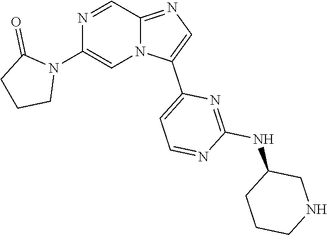 [SMILES: O=C1CCCN1c1cn2c(-c3ccnc(N[C@@H]4CCCNC4)n3)cnc2cn1] |
| 48-10 | 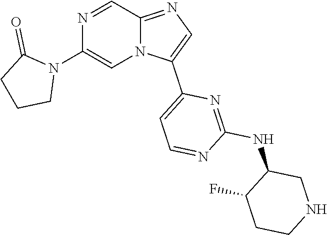 [SMILES: O=C1CCCN1c1cn2c(-c3ccnc(N[C@H]4CNCC[C@@H]4F)n3)cnc2cn1] |
| 49-10 | 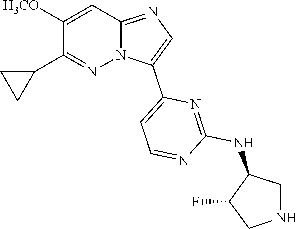 [SMILES: COc1cc2ncc(-c3ccnc(N[C@H]4CNC[C@@H]4F)n3)n2nc1C1CC1] |
| 50-10 | 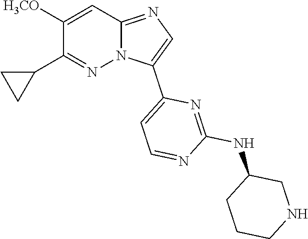 [SMILES: COc1cc2ncc(-c3ccnc(N[C@@H]4CCCNC4)n3)n2nc1C1CC1] |
| 51-10 | 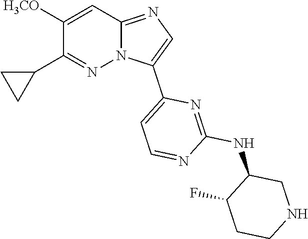 [SMILES: COc1cc2ncc(-c3ccnc(N[C@H]4CNCC[C@@H]4F)n3)n2nc1C1CC1] |
| 52-10 | 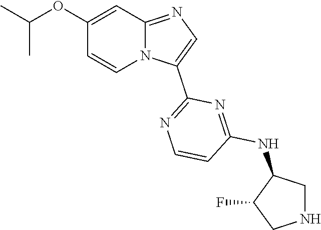 [SMILES: CC(C)Oc1ccn2c(-c3nccc(N[C@H]4CNC[C@@H]4F)n3)cnc2c1] |
| 53-10 | 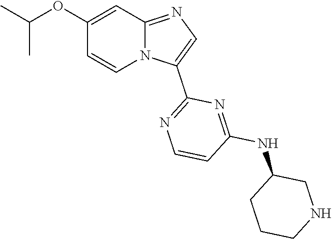 [SMILES: CC(C)Oc1ccn2c(-c3nccc(N[C@@H]4CCCNC4)n3)cnc2c1] |
| 54-10 | 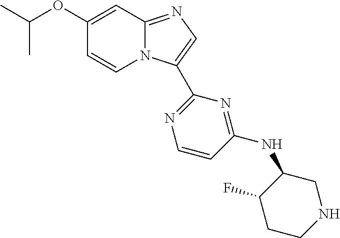 [SMILES: CC(C)Oc1ccn2c(-c3nccc(N[C@H]4CNCC[C@@H]4F)n3)cnc2c1] |
| 55-10 | 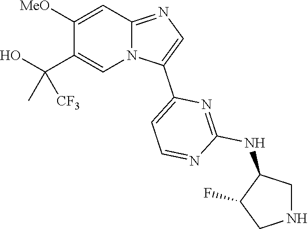 [SMILES: COc1cc2ncc(-c3ccnc(N[C@H]4CNC[C@@H]4F)n3)n2cc1C(C)(O)C(F)(F)F] |
| 56-10 | 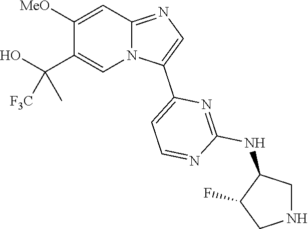 [SMILES: COc1cc2ncc(-c3ccnc(N[C@H]4CNC[C@@H]4F)n3)n2cc1C(C)(O)C(F)(F)F] |
| 57-10 | 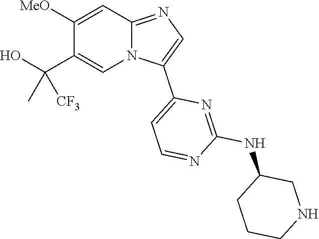 [SMILES: COc1cc2ncc(-c3ccnc(N[C@@H]4CCCNC4)n3)n2cc1C(C)(O)C(F)(F)F] |
| 58-10 | 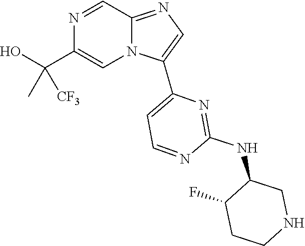 [SMILES: CC(O)(c1cn2c(-c3ccnc(N[C@H]4CNCC[C@@H]4F)n3)cnc2cn1)C(F)(F)F] |
| 59-10 | 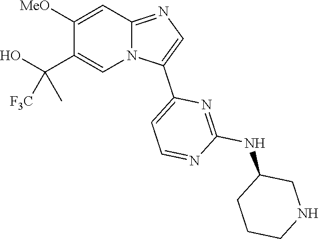 [SMILES: COc1cc2ncc(-c3ccnc(N[C@@H]4CCCNC4)n3)n2cc1C(C)(O)C(F)(F)F] |
| 60-10 | 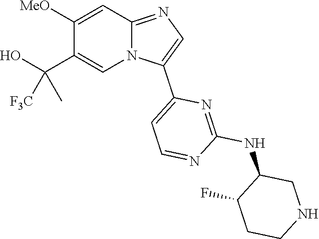 [SMILES: COc1cc2ncc(-c3ccnc(N[C@H]4CNCC[C@@H]4F)n3)n2cc1C(C)(O)C(F)(F)F] |
| 61-10 | 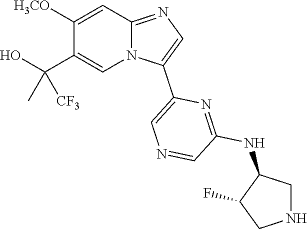 [SMILES: COc1cc2ncc(-c3cncc(N[C@H]4CNC[C@@H]4F)n3)n2cc1C(C)(O)C(F)(F)F] |
| 62-10 | 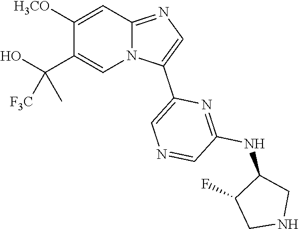 [SMILES: COc1cc2ncc(-c3cncc(N[C@H]4CNC[C@@H]4F)n3)n2cc1C(C)(O)C(F)(F)F] |
| 63-10 | 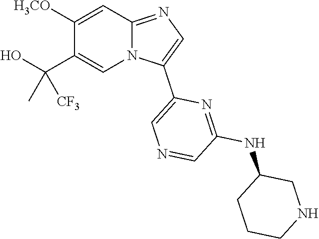 [SMILES: COc1cc2ncc(-c3cncc(N[C@@H]4CCCNC4)n3)n2cc1C(C)(O)C(F)(F)F] |
| 64-10 | 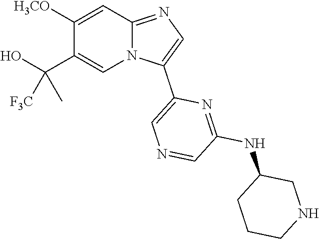 [SMILES: COc1cc2ncc(-c3cncc(N[C@@H]4CCCNC4)n3)n2cc1C(C)(O)C(F)(F)F] |
| 65-10 | 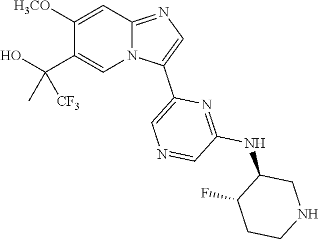 [SMILES: COc1cc2ncc(-c3cncc(N[C@H]4CNCC[C@@H]4F)n3)n2cc1C(C)(O)C(F)(F)F] |
| 66-10 | 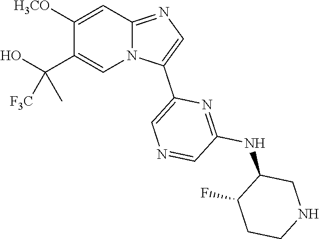 [SMILES: COc1cc2ncc(-c3cncc(N[C@H]4CNCC[C@@H]4F)n3)n2cc1C(C)(O)C(F)(F)F] |
| 67-10 | 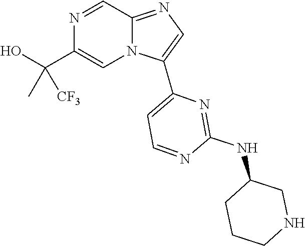 [SMILES: CC(O)(c1cn2c(-c3ccnc(N[C@@H]4CCCNC4)n3)cnc2cn1)C(F)(F)F] |
| 68-10 | 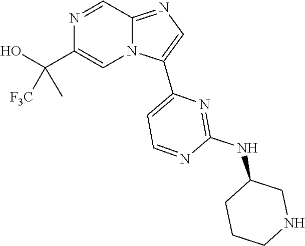 [SMILES: CC(O)(c1cn2c(-c3ccnc(N[C@@H]4CCCNC4)n3)cnc2cn1)C(F)(F)F] |
| 69-10 | 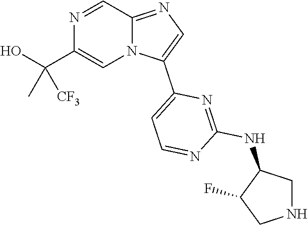 [SMILES: CC(O)(c1cn2c(-c3ccnc(N[C@H]4CNC[C@@H]4F)n3)cnc2cn1)C(F)(F)F] |
| 70-10 | 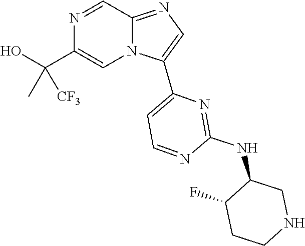 [SMILES: CC(O)(c1cn2c(-c3ccnc(N[C@H]4CNCC[C@@H]4F)n3)cnc2cn1)C(F)(F)F] |
| 71-10 | 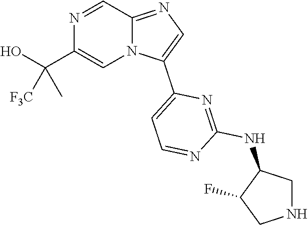 [SMILES: CC(O)(c1cn2c(-c3ccnc(N[C@H]4CNC[C@@H]4F)n3)cnc2cn1)C(F)(F)F] |
| 72-10 | 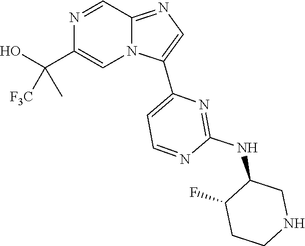 [SMILES: CC(O)(c1cn2c(-c3ccnc(N[C@H]4CNCC[C@@H]4F)n3)cnc2cn1)C(F)(F)F] |
| 73-10 | 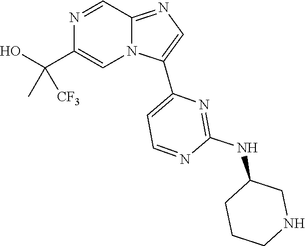 [SMILES: CC(O)(c1cn2c(-c3ccnc(N[C@@H]4CCCNC4)n3)cnc2cn1)C(F)(F)F] |
| 74-10 | 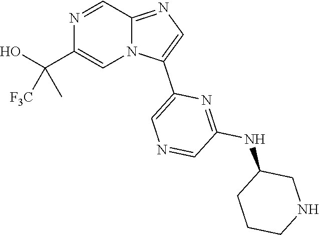 [SMILES: CC(O)(c1cn2c(-c3cncc(N[C@@H]4CCCNC4)n3)cnc2cn1)C(F)(F)F] |
| 75-10 | 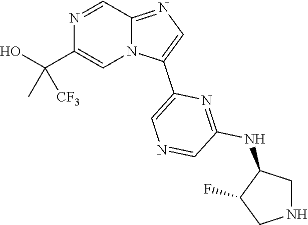 [SMILES: CC(O)(c1cn2c(-c3cncc(N[C@H]4CNC[C@@H]4F)n3)cnc2cn1)C(F)(F)F] |
| 76-10 | 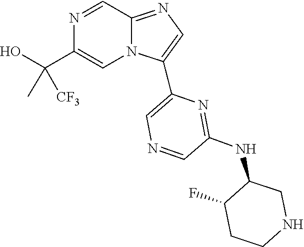 [SMILES: CC(O)(c1cn2c(-c3cncc(N[C@H]4CNCC[C@@H]4F)n3)cnc2cn1)C(F)(F)F] |
| 77-10 | 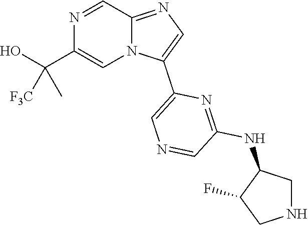 [SMILES: CC(O)(c1cn2c(-c3cncc(N[C@H]4CNC[C@@H]4F)n3)cnc2cn1)C(F)(F)F] |
| 78-10 | 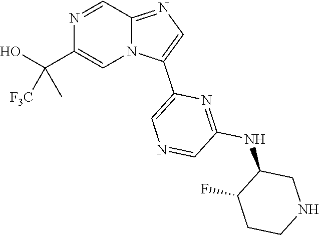 [SMILES: CC(O)(c1cn2c(-c3cncc(N[C@H]4CNCC[C@@H]4F)n3)cnc2cn1)C(F)(F)F] |
| 79-10 | 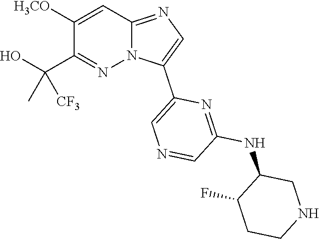 [SMILES: COc1cc2ncc(-c3cncc(N[C@H]4CNCC[C@@H]4F)n3)n2nc1C(C)(O)C(F)(F)F] |
| 80-10 | 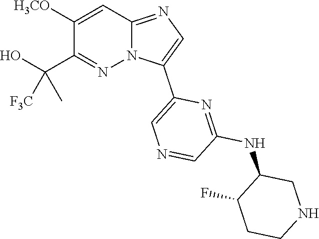 [SMILES: COc1cc2ncc(-c3cncc(N[C@H]4CNCC[C@@H]4F)n3)n2nc1C(C)(O)C(F)(F)F] |
| 81-10 | 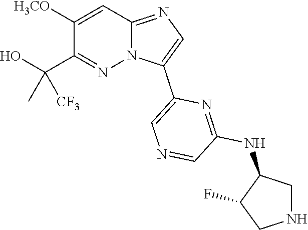 [SMILES: COc1cc2ncc(-c3cncc(N[C@H]4CNC[C@@H]4F)n3)n2nc1C(C)(O)C(F)(F)F] |
| 82-10 | 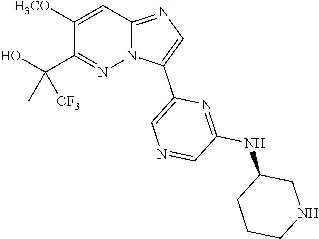 [SMILES: COc1cc2ncc(-c3cncc(N[C@@H]4CCCNC4)n3)n2nc1C(C)(O)C(F)(F)F] |
| 83-10 | 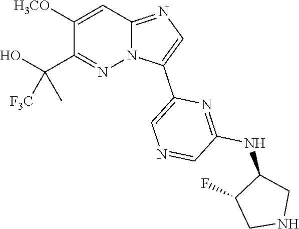 [SMILES: COc1cc2ncc(-c3cncc(N[C@H]4CNC[C@@H]4F)n3)n2nc1C(C)(O)C(F)(F)F] |
| 84-10 | 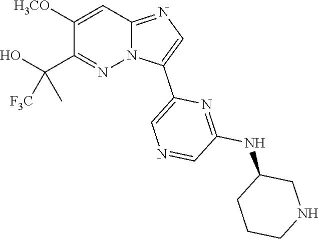 [SMILES: COc1cc2ncc(-c3cncc(N[C@@H]4CCCNC4)n3)n2nc1C(C)(O)C(F)(F)F] |

In one embodiment, the compound is selected from:

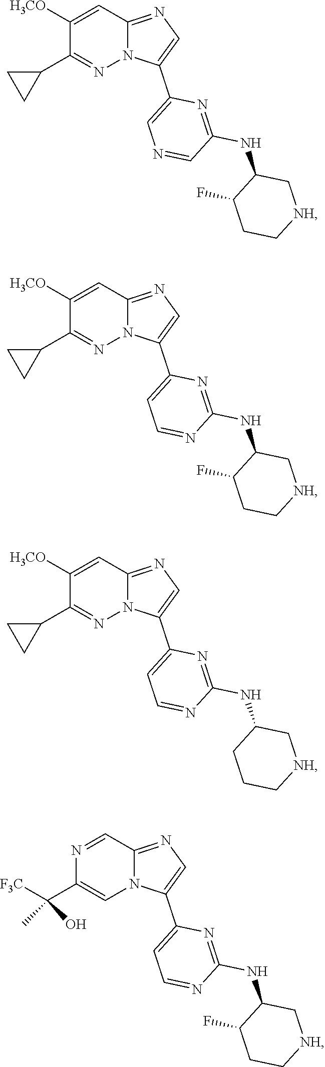 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F]

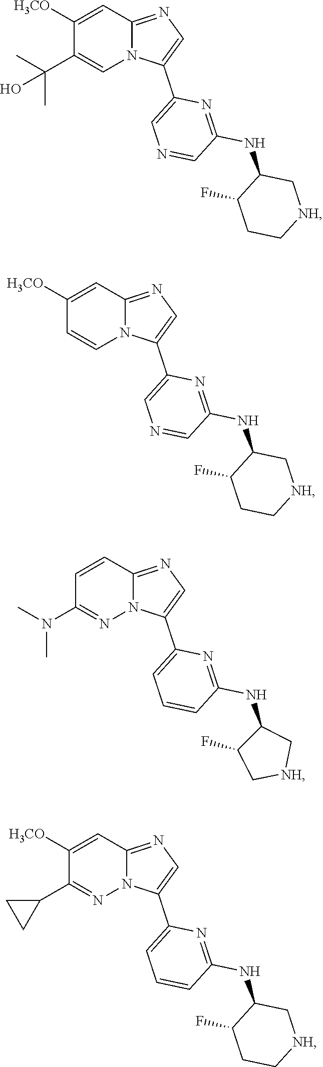 [SMILES: COc1cc2ncc(-c3cncc(N[C@H]4CNCC[C@@H]4F)n3)n2cc1C(C)(C)O ; COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1]

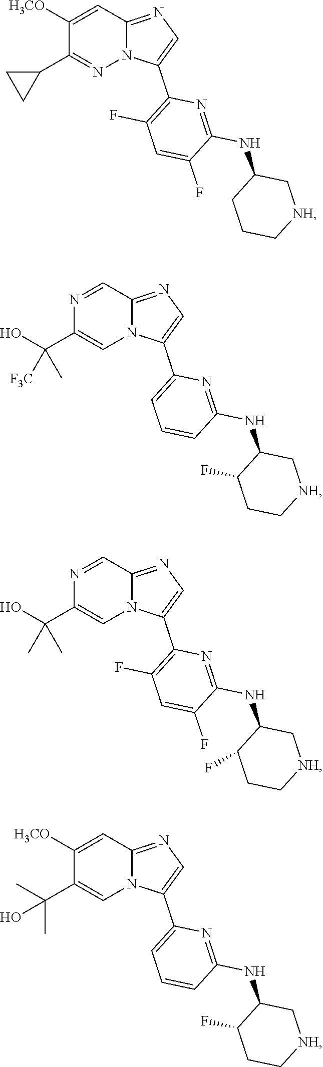 [SMILES: COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F ; CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O]

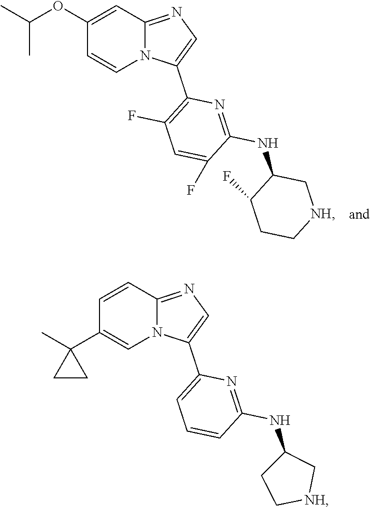 [SMILES: CC(C)Oc1ccn2c(-c3nc(N[C@H]4CNCC[C@@H]4F)c(F)cc3F)cnc2c1 ; "and" ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

In one embodiment, the compound is selected from:

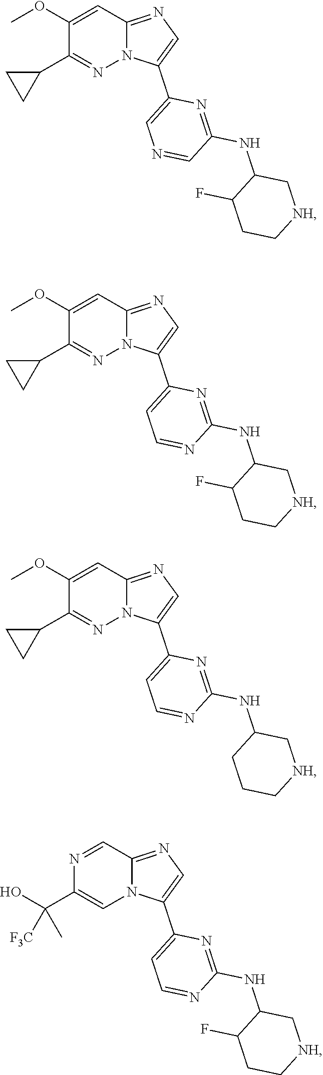 [SMILES: COc1cc2ncc(-c3cncc(NC4CNCCC4F)n3)n2nc1C1CC1 ; COc1cc2ncc(-c3ccnc(NC4CNCCC4F)n3)n2nc1C1CC1 ; COc1cc2ncc(-c3ccnc(NC4CCCNC4)n3)n2nc1C1CC1 ; CC(O)(c1cn2c(-c3ccnc(NC4CNCCC4F)n3)cnc2cn1)C(F)(F)F]

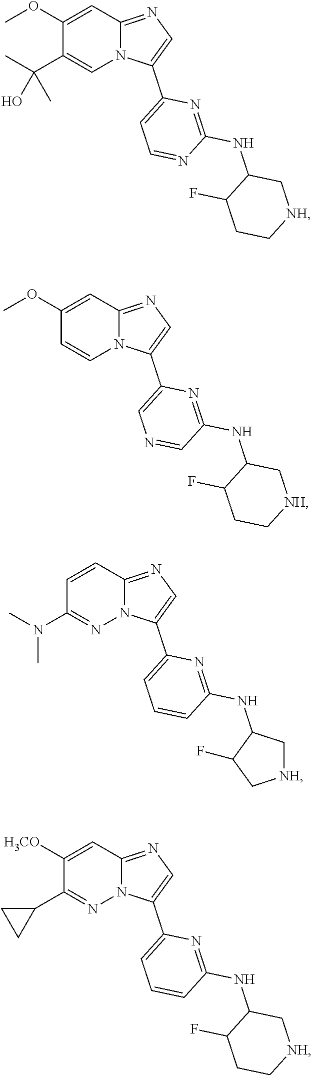 [SMILES: COc1cc2ncc(-c3ccnc(NC4CNCCC4F)n3)n2cc1C(C)(C)O ; COc1ccn2c(-c3cncc(NC4CNCCC4F)n3)cnc2c1 ; CN(C)c1ccc2ncc(-c3cccc(NC4CNCC4F)n3)n2n1 ; COc1cc2ncc(-c3cccc(NC4CNCCC4F)n3)n2nc1C1CC1]

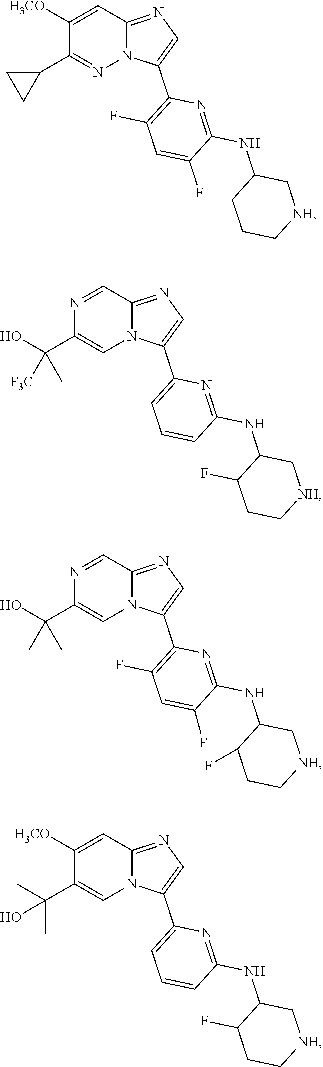 [SMILES: COc1cc2ncc(-c3nc(NC4CCCNC4)c(F)cc3F)n2nc1C1CC1 ; CC(O)(c1cn2c(-c3cccc(NC4CNCCC4F)n3)cnc2cn1)C(F)(F)F ; CC(C)(O)c1cn2c(-c3nc(NC4CNCCC4F)c(F)cc3F)cnc2cn1 ; COc1cc2ncc(-c3cccc(NC4CNCCC4F)n3)n2cc1C(C)(C)O]

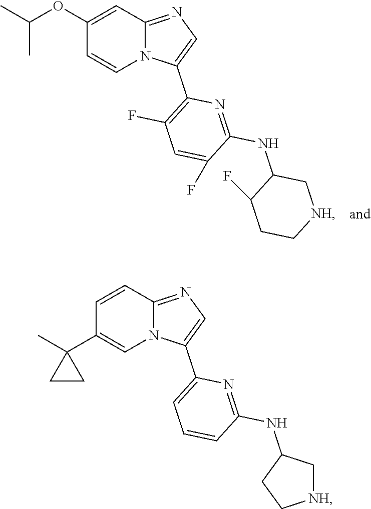 [SMILES: CC(C)Oc1ccn2c(-c3nc(NC4CNCCC4F)c(F)cc3F)cnc2c1 ; "and" ; CC1(c2ccc3ncc(-c4cccc(NC5CCNC5)n4)n3c2)CC1]

or a salt, ester, solvate, optical isomer, geometric isomer, or salt of an isomer of any one thereof.

### Methods

In another aspect, the present disclosure provides a method of treating an inflammatory disease/disorder, acute myeloid leukemia (AML), or myelodysplastic syndrome (MDS) in a subject comprising administering to the subject a compound that inhibits IRAK1 and IRAK4. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the subject has AML or is suspected of having AML. In one embodiment, the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AML. In one embodiment, the subject has MDS or is suspected of having MDS.

In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the IRAK4:IRAK1 potency ratio is calculated from the IRAK1 and IRAK4 IC₅₀ measurements. In one embodiment, the compound modifies the expression of one or more IRAK1/4-associated genes found to be differentially expressed in the subject compared to a healthy control subject. In one embodiment, the compound modifies the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be differentially expressed in the subject compared to a healthy control subject. In one embodiment, compound decreases the expression of one of more IRAK1/4-associated genes found to be elevated in the subject compared to the healthy control subject. In one embodiment, compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be elevated in the subject compared to the healthy control subject. In one embodiment, the compound increases the expression of one or more IRAK1/4-associated genes found to be decreased in the subject compared to the healthy control subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be decreased in the subject compared to the healthy control subject. In one embodiment, the compound decreases the expression of one or more IRAK1/4-associated genes selected from: KRAS, SOX5, H1MX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits IRAK1, IRAK4, and FLT3. In one embodiment, the compound inhibits FLT3. In one embodiment, the compound treats AML and/or MDS in the subject by suppressing leukemic stem/progenitor cell (LSPC) function and inducing differentiation. In one embodiment, the inhibition of IRAK1 and IRAK4 by the compound of the disclosure promotes the suppression of LSPC function and the induction of differentiation in the subject with AML and/or MDS.

In one embodiment, the compound provides sustained treatment of the inflammatory disease/disorder, AML, or MDS in the subject. In one embodiment, the sustained treatment is greater than the treatment provided from a compound which inhibits IRAK1 without inhibiting IRAK4 or inhibits IRAK4 without inhibiting TRAK1. In one embodiment, the compound continues to treat the inflammatory disease/disorder, AML, or MDS in the subject after the administration is stopped. In one embodiment, the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped. In one embodiment, the compound treats MDS or AML. In one embodiment, the compound is a compound of any one of Formulas (I-5007), (IIf-5007)-(IIk-5007), (IIm-5007)-(IIt-5007), (IlIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (11-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

In another aspect, the present disclosure provides a method of determining a compound that is effective at treating an inflammatory disease/disorder, AML, or MDS, comprising:
determining from the IRAK1 IC₅₀ and IRAK4 IC₅₀ of the compound that the compound inhibits IRAK1 and IRAK4;
calculating from the IRAK1 and IRAK4 IC₅₀ of the compound that the compound has an IRAK4:IRAK1 potency ratio of less than about 40; and
administering the compound to a subject with an inflammatory disease/disorder, AML, or MDS, thus treating the inflammatory disease/disorder, AML, or MDS.

In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the subject has AML or is suspected of having AML. In one embodiment, the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AML. In one embodiment, the subject has MDS or is suspected of having MDS.

In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits FLT3. In one embodiment, the compound treats AML and/or MDS in the subject by suppressing leukemic stem/progenitor cell (LSPC) function and inducing differentiation. In one embodiment, the inhibition of IRAK1 and IRAK4 by the compound of the disclosure promotes the suppression of LSPC function and the induction of differentiation in the subject with AML and/or MDS.

In one embodiment, the compound provides sustained treatment of the inflammatory disease/disorder, AML, or MDS in the subject. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in the subject after the administration is stopped. In one embodiment, the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped. In one embodiment, the compound treats MDS or AML. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

In yet another aspect, the present disclosure provides a method of determining a compound that is effective at treating an inflammatory disease/disorder, acute myeloid leukemia (AML), or myelodysplastic syndrome (MDS), comprising: determining that the compound is an IRAK1/4 inhibitor which modifies the expression of one of more IRAK1/4-associated genes found to be differentially expressed in a subject with an inflammatory disease/disorder, AML, or MDS compared to a healthy control subject; and administering the compound to the subject, treating the inflammatory disease/disorder, AML, or MDS.

In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the subject has AML or is suspected of having AML. In one embodiment, the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AML. In one embodiment, the subject has MDS or is suspected of having MDS.

In one embodiment, the compound decreases the expression of one or more IRAK1/4-associated genes found to be elevated in the subject compared to the healthy control subject. In one embodiment, the compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be elevated in the subject compared to the healthy control subject. In one embodiment, the compound increases the expression of one or more IRAK1/4-associated genes found to be decreased in the subject compared to the healthy control subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be decreased in the subject compared to the healthy control subject. In one embodiment, the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF1, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one or more IRAK1/4-associated genes selected from: KRAS, SOX5, HMEX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits FLT3. In one embodiment, the compound treats AML and/or MDS in the subject by suppressing leukemic stem/progenitor cell (LSPC) function and inducing differentiation. In one embodiment, the inhibition of IRAK1 and IRAK4 by the compound of the disclosure promotes the suppression of LSPC function and the induction of differentiation in the subject with AML and/or MDS.

In one embodiment, the compound provides sustained treatment of the inflammatory disease/disorder, AML, or MDS in the subject. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in the subject after the administration is stopped. In one embodiment, the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped. In one embodiment, the compound treats MDS or AML. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (I-5010), (Ia-5010)-(Id-5010), (1-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

In yet another aspect, the present disclosure provides a method of determining a subject with a disease/disorder that can be treated by the administration of a compound which inhibits IRAK1 and IRAK4, comprising:
determining that a subject with an inflammatory disease/disorder, AML, or MDS has elevated IRAK1 expression, IRAK4 expression, FLT3 expression, NF-kB signaling, TLR signaling, IL-1 signaling, or a combination thereof, and
administering to the subject a compound that inhibits IRAK1 and IRAK4, wherein the compound has an IRAK4:IRAK1 potency ratio of less than about 40, as calculated from the IRAK1 and IRAK4 IC₅₀ of the compound, thus treating the inflammatory disease/disorder, AML, or MDS.

In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the subject has AML or is suspected of having AML. In one embodiment, the AML is relapsed AML, refractory AML, relapsed/refractory AMVL, AML with resistance to hypomethylating agents, AML with resistance to venetoclax, AMIL with resistance to hypomethylating agents and venetoclax, monocytic AML, or monocytic-like AML. In one embodiment, the subject has MDS or is suspected of having MDS.

In one embodiment, the subject has elevated IRAK1 expression, IRAK4 expression, or a combination thereof. In one embodiment, the subject has elevated FLT3 expression. In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits FLT3. In one embodiment, the compound inhibits IRAK1, IRAK4, and FLT3. In one embodiment, the compound treats AML and/or MDS in the subject by suppressing leukemic stem/progenitor cell (LSPC) function and inducing differentiation. In one embodiment, the inhibition of IRAK1 and IRAK4 by the compound of the disclosure promotes the suppression of LSPC function and the induction of differentiation in the subject with AML and/or MDS.

In one embodiment, the compound provides sustained treatment of the inflammatory disease/disorder, AML, or MDS in the subject. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in the subject after the administration is stopped. In one embodiment, the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped. In one embodiment, the compound treats MDS or AML. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

In yet another aspect, the present disclosure provides a method of determining a subject with a disease/disorder that can be treated by the administration of a compound which inhibits IRAK1 and IRAK4, comprising:
determining that a subject with an inflammatory disease/disorder, acute myeloid leukemia (AML), or myelodysplastic syndrome (MDS) has one of more IRAK1/4-associated genes that are differentially expressed compared to a healthy control subject; and
administering to the subject a compound that inhibits IRAK1 and IRAK4, thus treating the inflammatory disease/disorder, AML, or MDS.

In one embodiment, the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis. In one embodiment, the subject has AML or is suspected of having AML. In one embodiment, the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AML. In one embodiment, the subject has MDS or is suspected of having MDS.

In one embodiment, the compound modifies the expression of one or more IRAK1/4-associated genes found to be differentially expressed in the subject compared to the healthy control subject. In one embodiment, the compound modifies the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 more IRAK1/4-associated genes found to be differentially expressed in the subject compared to the healthy control subject. In one embodiment, the compound decreases the expression of one of more IRAK1/4-associated genes found to be elevated in the subject compared to the healthy control subject. In one embodiment, the compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 TRAK1/4-associated genes found to be elevated in the subject compared to the healthy control subject. In one embodiment, the compound increases the expression of one or more IRAK1/4-associated genes found to be decreased in the subject compared to the healthy control subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes found to be decreased in the subject compared to the healthy control subject. In one embodiment, the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound decreases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject. In one embodiment, the compound increases the expression of one, two, three, four, five, six, seven, eight, nine, ten, eleven, twelve, thirteen, fourteen, fifteen, sixteen, seventeen, eighteen, nineteen, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

In one embodiment, the compound has an IRAK1 IC₅₀ of less than about 75 nM, less than about 65 nM, less than about 55 nM, less than about 45 nM, less than about 35 nM, less than about 25 nM, less than about 15 nM, or less than about 5 nM. In one embodiment, the compound has an IRAK4 IC₅₀ of less than about 50 nM, less than about 40 nM, less than about 30 nM, less than about 20 nM, less than about 10 nM, less than about 5 nM, or less than about 1 nM. In one embodiment, the compound has IRAK4:IRAK1 potency ratio of less than about 70, less than about 60, less than about 50, less than about 40, less than about 30, or less than about 20. In one embodiment, the compound inhibits NF-kB-mediated signaling. In one embodiment, inhibition of IRAK1 and IRAK4 inhibits NF-kB-mediated signaling. In one embodiment, the compound inhibits TLR signaling and IL-1 signaling. In one embodiment, the inhibition of IRAK1 and IRAK4 inhibits TLR signaling and IL-1 signaling. In one embodiment, the compound inhibits FLT3. In one embodiment, the compound inhibits IRAK1, IRAK4, and FLT3. In one embodiment, the compound treats AML and/or MDS in the subject by suppressing leukemic stem/progenitor cell (LSPC) function and inducing differentiation. In one embodiment, the inhibition of IRAK1 and IRAK4 by the compound of the disclosure promotes the suppression of LSPC function and the induction of differentiation in the subject with AML and/or MDS.

In one embodiment, the compound provides sustained treatment of the inflammatory disease/disorder, AML, or MDS in the subject. In one embodiment, the sustained treatment is greater than the treatment provided from a compound which inhibits IRAK1 without inhibiting IRAK4 or inhibits IRAK4 without inhibiting IRAK1. In one embodiment, the compound continues to treat the inflammatory disease/disorder, AML, or MDS in the subject after the administration is stopped. In one embodiment, the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped. In one embodiment, the compound treats MDS or AML. In one embodiment, the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof. In one embodiment, the compound is selected from Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

### CLAUSES OF THE DISCLOSURE

The following clauses describe certain embodiments.

Clause 1. A method of treating an inflammatory disease/disorder, acute myeloid leukemia (AML), or myelodysplastic syndrome (MDS) in a subject comprising administering to the subject a compound that inhibits IRAK1 and IRAK4.

Clause 2. The method of clause 1, wherein the compound has an IRAK1 IC₅₀ of less than about 75 nM.

Clause 3. The method of clause 1 or 2, wherein the compound has an IRAK4 IC₅₀ of less than about 10 nM.

Clause 4. The method of any one of clauses 1 to 3, wherein the compound has IRAK4:IRAK1 potency ratio of less than about 40.

Clause 5. The method of any one of clauses 1 to 4, wherein the compound inhibits NF-kB-mediated signaling.

Clause 6. The method of clause 5, wherein inhibition of IRAK1 and IRAK4 inhibits NF-kB-mediated signaling.

Clause 7. The method of any one of clauses 1 to 6, wherein the compound inhibits TLR signaling and IL-1 signaling.

Clause 8. The method of clause 7, wherein the inhibition of IRAK1 and IRAK4 inhibits TLR signaling and IL-1 signaling.

Clause 9. The method of any one of clauses 1 to 8, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 10. The method of clause 9, wherein the inhibition of IRAK1 and IRAK4 inhibits cancer cell colony formation and/or cancer progenitor cell function.

Clause 11. The method of any one of clauses 1 to 10, wherein the compound provides sustained treatment of the inflammatory disease/disorder, AML, or MDS in the subject.

Clause 12. The method of clause 11, wherein the sustained treatment is greater than the treatment provided from a compound which inhibits IRAK1 without inhibiting IRAK4 or inhibits IRAK4 without inhibiting IRAK1.

Clause 13. The method of any one of clauses 1 to 12, wherein the compound continues to treat the inflammatory disease/disorder, AML, or MDS in the subject after the administration is stopped.

Clause 14. The method of clause 13, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 15. The method of any one of clauses 1 to 14, wherein the compound treats MDS or AML.

Clause 16a. The method of any one of clauses 1 to 15, wherein the compound is a compound of Formula (I-5007), (VI-5003), (VII-5008), (I-5009), (I-5010), (II-5010), (1II-5010), or a salt, ester, solvate, optical isomer, geometric isomer, or salt of an isomer of any one thereof.

Clause 16b. The method of any one of clauses 1 to 16a, wherein the compound is selected from any one of Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

Clause 17. The method of any one of clauses 1 to 16, wherein the compound is selected from:

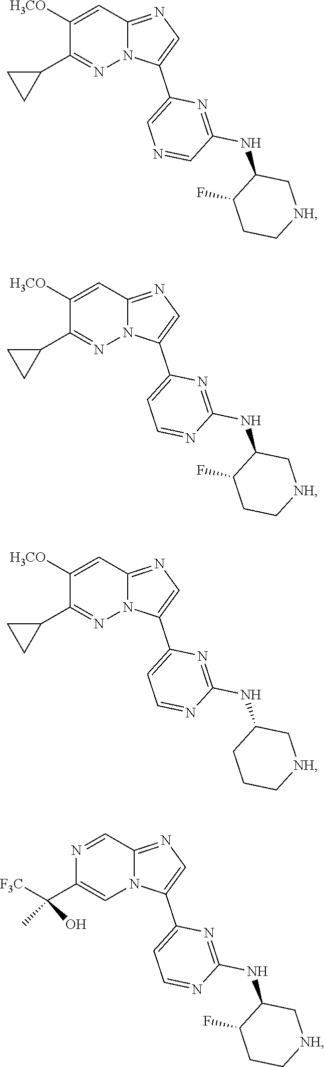 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F]

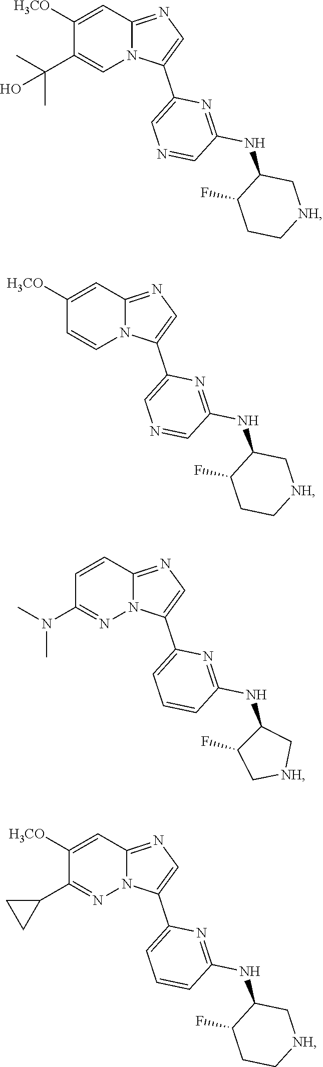 [SMILES: COc1cc2ncc(-c3cncc(N[C@H]4CNCC[C@@H]4F)n3)n2cc1C(C)(C)O ; COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1]

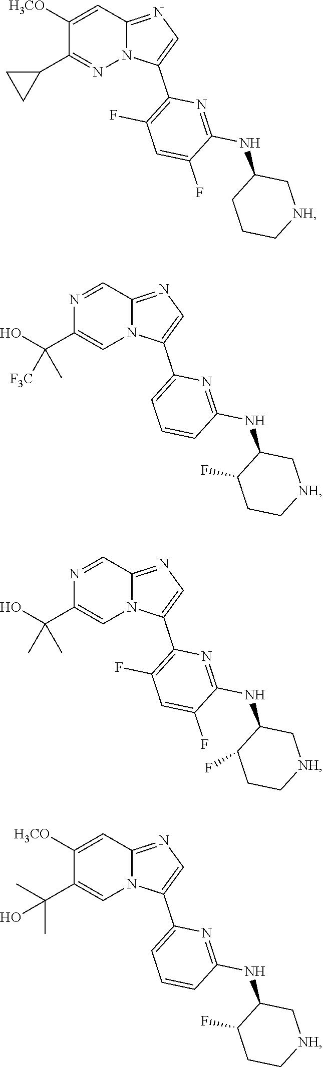 [SMILES: COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F ; CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O]

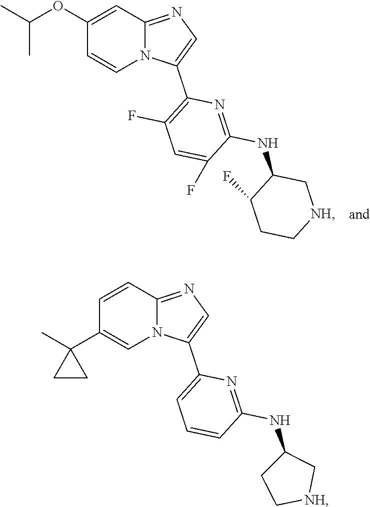 [SMILES: CC(C)Oc1ccn2c(-c3nc(N[C@H]4CNCC[C@@H]4F)c(F)cc3F)cnc2c1 ; "and" ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 18. A method of determining a compound that is effective at treating an inflammatory disease/disorder, acute myeloid leukemia (AML), or myelodysplastic syndrome (MDS), comprising:
determining from the IRAK1 IC₅₀ and IRAK4 IC₅₀ of the compound that the compound inhibits IRAK1 and IRAK4;
calculating from the IRAK1 and IRAK4 IC₅₀ of the compound that the compound has an IRAK4:IRAK1 potency ratio of less than about 40; and
administering the compound to a subject with an inflammatory disease/disorder, AML, or MDS, treating the inflammatory disease/disorder, AML, or MDS.

Clause 19. The method of clause 18, wherein the compound has an IRAK1 IC₅₀ of less than about 75 nM.

Clause 20. The method of clause 18 or 19, wherein the compound has an IRAK4 IC₅₀ of less than about 10 nM.

Clause 21. The method of any one of clauses 18 to 20, wherein the compound inhibits NF-kB-mediated signaling.

Clause 22. The method of clause 21, wherein inhibition of IRAK1 and IRAK4 inhibits NF-kB-mediated signaling.

Clause 23. The method of any one of clauses 18 to 22, wherein the compound inhibits TLR signaling and IL-1 signaling.

Clause 24. The method of clause 23, wherein the inhibition of RAK1 and IRAK4 inhibits TLR signaling and IL-1 signaling.

Clause 25. The method of any one of clauses 18 to 24, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 26. The method of clause 25, wherein the inhibition of IRAK1 and IRAK4 inhibits cancer cell colony formation and/or cancer progenitor cell function.

Clause 27. The method of any one of clauses 18 to 26, wherein the compound provides sustained treatment of the inflammatory disease/disorder, AML, or MDS in the subject.

Clause 28. The method of clause 27, wherein the sustained treatment is greater than the treatment provided from a compound which inhibits IRAK1 without inhibiting IRAK4 or inhibits IRAK4 without inhibiting IRAK1.

Clause 29. The method of any one of clauses 18 to 28, wherein the compound continues to treat the inflammatory disease/disorder, AML, or MDS in the subject after the administration is stopped.

Clause 30. The method of clause 29, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 31. The method of any one of clauses 18 to 30, wherein the compound treats MDS or AML.

Clause 32. The method of any one of clauses 18 to 31, wherein the compound is a compound of Formula (I-5007), (VI-5003), (VII-5008), (I-5009), (I-5010), (II-5010), (III-5010), or a salt, ester, solvate, optical isomer, geometric isomer, or salt of an isomer of any one thereof.

Clause 33. The method of any one of clauses 18 to 32, wherein the compound is selected from:

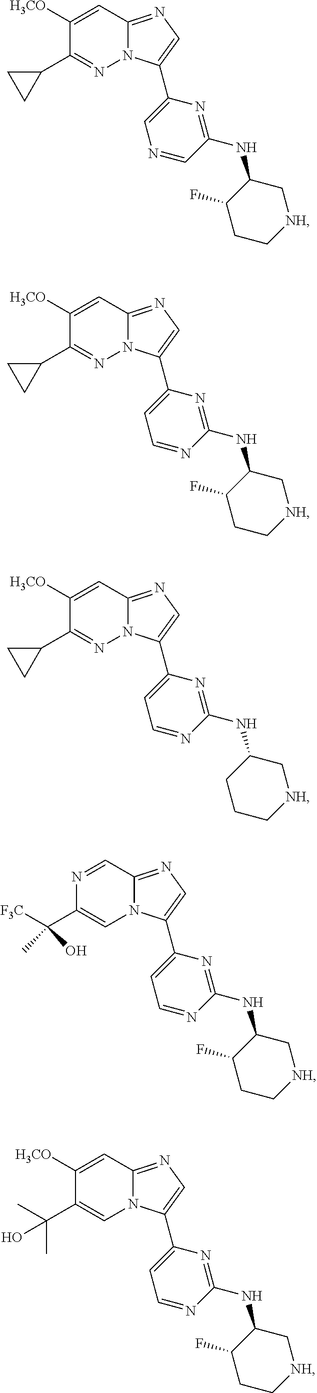 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F ; COc1cc2ncc(-c3ccnc(N[C@H]4CNCC[C@@H]4F)n3)n2cc1C(C)(C)O]

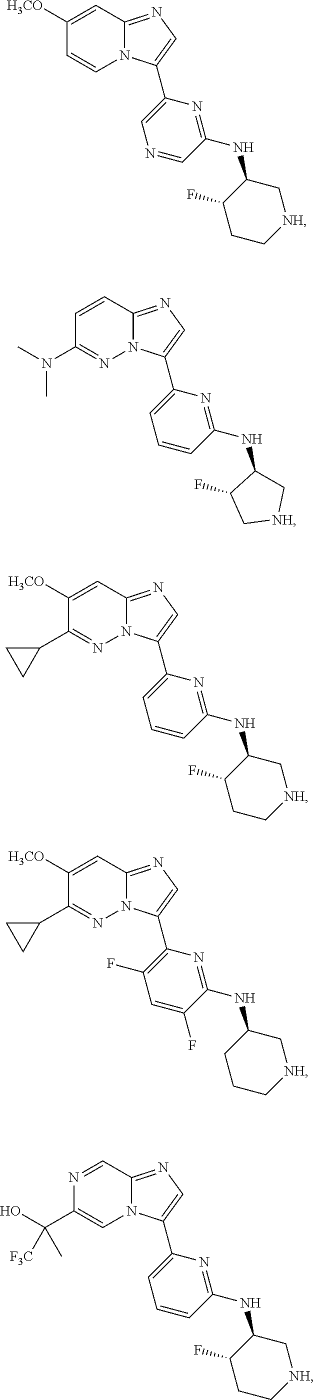 [SMILES: COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1 ; COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F]

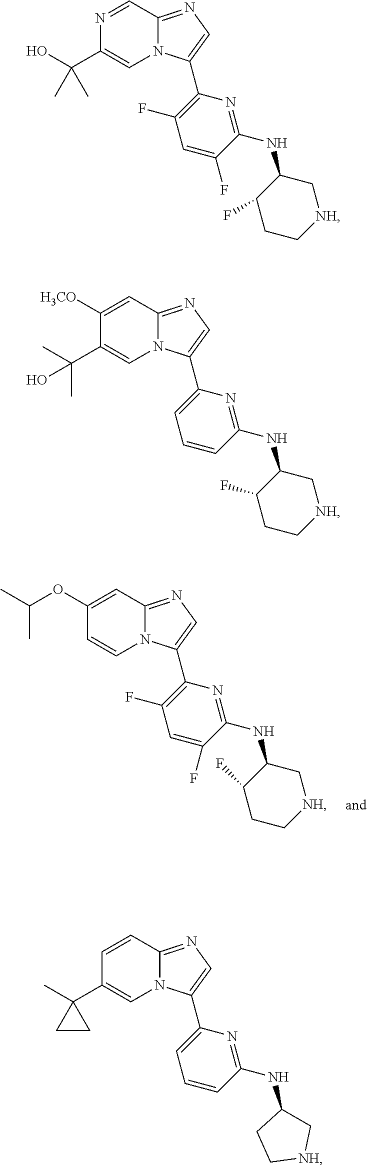 [SMILES: CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O ; CC(C)Oc1ccn2c(-c3nc(N[C@H]4CNCC[C@@H]4F)c(F)cc3F)cnc2c1 ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 34. A method of determining a subject with a disease/disorder that can be treated by the administration of a compound which inhibits IRAK1 and IRAK4, comprising:
determining that a subject with an inflammatory disease/disorder, acute myeloid leukemia (AML), or myelodysplastic syndrome (MDS) has elevated IRAK1 expression, IRAK4 expression, FLT3 expression, NF-kB signaling, TLR signaling, IL-1 signaling, or a combination thereof, and
administering to the subject a compound that inhibits IRAK1 and IRAK4 wherein the compound has an IRAK4:IRAK1 potency ratio of less than about 40, as calculated from the IRAK1 and IRAK4 IC₅₀ of the compound, thus treating the inflammatory disease/disorder, AML, or MDS.

Clause 35. The method of clause 34, wherein the subject has elevated IRAK1 expression, IRAK4 expression, or a combination thereof.

Clause 36. The method of clause 34 or 35, wherein the subject has elevated FLT3 expression.

Clause 37. The method of any one of clauses 34 to 36, wherein the compound has an IRAK1 IC₅₀ of less than about 75 nM.

Clause 38. The method of any one of clauses 34 to 37, wherein the compound has an IRAK4 IC₅₀ of less than about 10 nM.

Clause 39. The method of any one of clauses 34 to 38, wherein the compound inhibits NF-kB-mediated signaling.

Clause 40. The method of clause 39, wherein inhibition of IRAK1 and IRAK4 inhibits NF-kB-mediated signaling.

Clause 41. The method of any one of clauses 34 to 40, wherein the compound inhibits TLR signaling and IL-1 signaling.

Clause 42. The method of clause 41, wherein the inhibition of RAK1 and IRAK4 inhibits TLR signaling and IL-1 signaling.

Clause 43. The method of any one of clauses 34 to 42, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 44. The method of clause 43, wherein the inhibition of IRAK1 and IRAK4 inhibits cancer cell colony formation and/or cancer progenitor cell function.

Clause 45. The method of any one of clauses 34 to 44, wherein the compound provides sustained treatment of the inflammatory disease/disorder, AML, or MDS in the subject.

Clause 46. The method of clause 45, wherein the sustained treatment is greater than the treatment provided from a compound which inhibits IRAK1 without inhibiting IRAK4 or inhibits IRAK4 without inhibiting IRAK1.

Clause 47. The method of any one of clauses 34 to 46, wherein the compound continues to treat the inflammatory disease/disorder, AML, or MDS in the subject after the administration is stopped.

Clause 48. The method of clause 47, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 49. The method of any one of clauses 34 to 48, wherein the compound treats MDS or AML.

Clause 50. The method of any one of clauses 34 to 49, wherein the compound is a compound of Formula (I-5007), (VI-5003), (VII-5008), (I-5009), (I-5010), (II-5010), (III-5010), or a salt, ester, solvate, optical isomer, geometric isomer, or salt of an isomer of any one thereof.

Clause 51. The method of any one of clauses 34 to 50, wherein the compound is selected from:

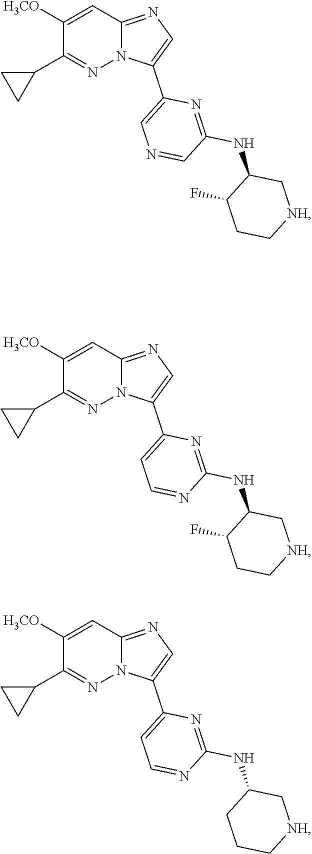 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1]

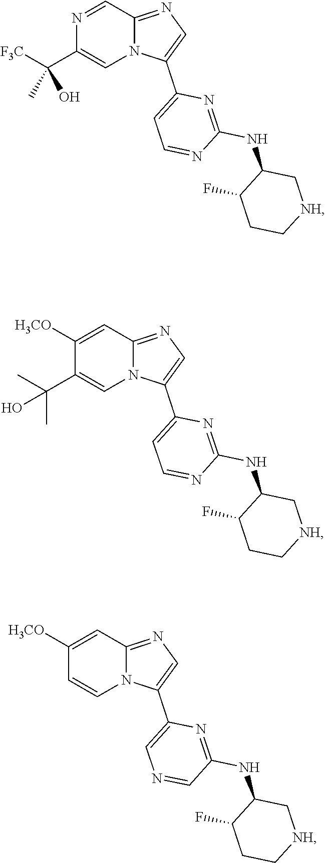 [SMILES: C[C@@](O)(c1cn2c(-c3ccnc(N[C@H]4CNCC[C@@H]4F)n3)cnc2cn1)C(F)(F)F ; COc1cc2ncc(-c3ccnc(N[C@H]4CNCC[C@@H]4F)n3)n2cc1C(C)(C)O ; COc1ccn2c(-c3cncc(N[C@H]4CNCC[C@@H]4F)n3)cnc2c1]

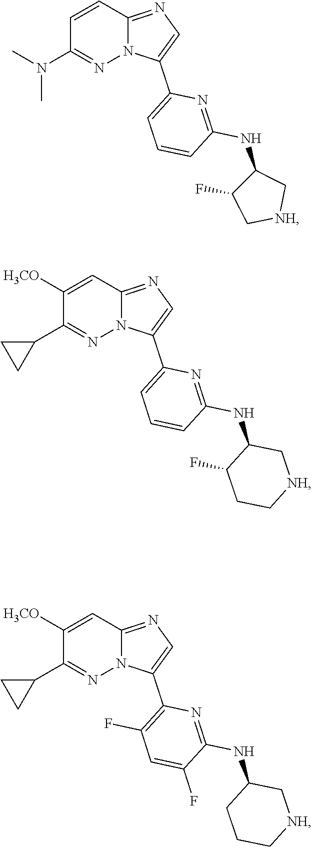 [SMILES: CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1 ; COc1cc2ncc(-c3nc(N[C@@H]4CCCNC4)c(F)cc3F)n2nc1C1CC1]

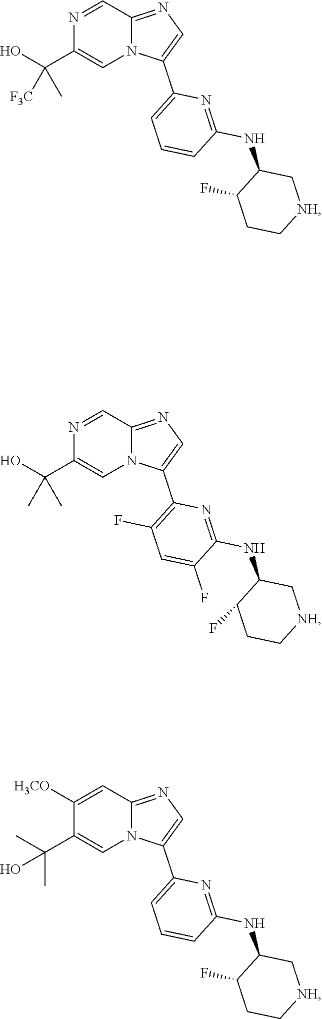 [SMILES: CC(O)(c1cn2c(-c3cccc(N[C@H]4CNCC[C@@H]4F)n3)cnc2cn1)C(F)(F)F ; CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O]

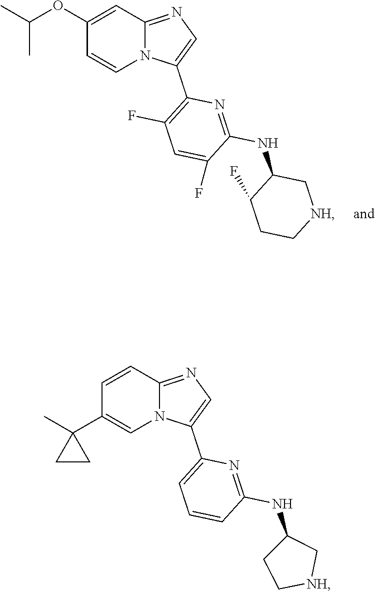 [SMILES: CC(C)Oc1ccn2c(-c3nc(N[C@H]4CNCC[C@@H]4F)c(F)cc3F)cnc2c1 ; "and" ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 101. A method of treating an inflammatory disease/disorder, acute myeloid leukemia (AML), or myelodysplastic syndrome (MDS) in a subject in need thereof, the method comprising administering to the subject a compound that inhibits IRAK1 and IRAK4.

Clause 102. The method of clause 101, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

Clause 103. The method of clause 101, wherein the AML is relapsed AML, refractory AML, relapsed/refractory AML, AML with resistance to hypomethylating agents, AML with resistance to venetoclax, AIL with resistance to hypomethylating agents and venetoclax, monocytic AML, or monocytic-like AML.

Clause 104. The method of any one of clauses 101 to 103, wherein the compound has an IRAK1 IC₅₀ of less than about 75 nM.

Clause 105. The method of any one of clauses 101 to 104, wherein the compound has an IRAK4 IC₅₀ of less than about 10 nM.

Clause 106. The method of any one of clauses 101 to 105, wherein the compound has IRAK4:IRAK1 potency ratio of less than about 40.

Clause 107. The method of any one of clauses 101 to 106, wherein the subject in need thereof has elevated expression of one or more IRAK1/4-associated genes compared to a healthy control subject and/or decreased expression of one or more IRAK1/4-associated genes compared to a healthy control subject.

Clause 108. The method of clause 107, wherein the subject in need thereof has elevated expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

Clause 109. The method of clause 107 or 108, wherein the subject in need thereof has decreased expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

Clause 110. The method of any one of clauses 107 to 109, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

Clause 111. The method of clause 110, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 112. The method of clause 110 or 111, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 113. The method of any one of clauses 101 to 112, wherein the compound inhibits NF-kB-mediated signaling.

Clause 114. The method of clause 113, wherein inhibition of IRAK1 and IRAK4 inhibits NF-kB-mediated signaling.

Clause 115. The method of any one of clauses 101 to 114, wherein the compound inhibits TLR signaling and IL-1 signaling.

Clause 116. The method of clause 115, wherein the inhibition of RAK1 and IRAK4 inhibits TLR signaling and IL-1 signaling.

Clause 117. The method of any one of clauses 101 to 116, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 118. The method of clause 117, wherein the inhibition of RAK1 and IRAK4 inhibits cancer cell colony formation and/or cancer progenitor cell function.

Clause 119. The method of any one of clauses 101 to 118, wherein the compound provides sustained treatment of the inflammatory disease/disorder, AML, or MDS in the subject.

Clause 120. The method of clause 119, wherein the sustained treatment is greater than the treatment provided from a compound which inhibits IRAK1 without inhibiting IRAK4 or inhibits IRAK4 without inhibiting IRAK1.

Clause 121. The method of any one of clauses 101 to 120, wherein the compound continues to treat the inflammatory disease/disorder, AML, or MDS in the subject after the administration is stopped.

Clause 122. The method of clause 121, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 123. The method of any one of clauses 101 to 122, wherein the compound treats MDS or AML.

Clause 124. The method of any one of clauses 101 to 123, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (1Ip-5008)-(IIu-5008), (IIIq-5008)-(IIIs-5008), (I-5009), (IIf-5009)-(IIj-5009), (I-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

Clause 125. The method of any one of clauses 101 to 124, wherein the compound is selected from:

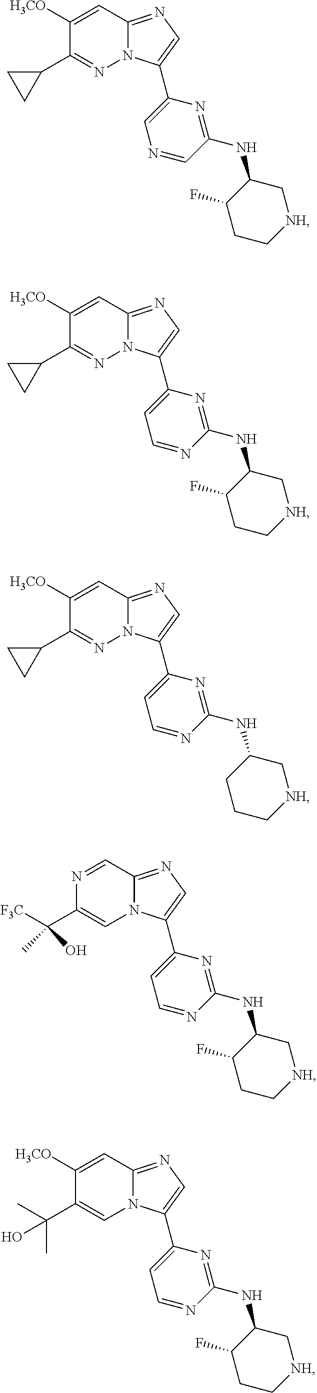 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F ; COc1cc2ncc(-c3ccnc(N[C@H]4CNCC[C@@H]4F)n3)n2cc1C(C)(C)O]

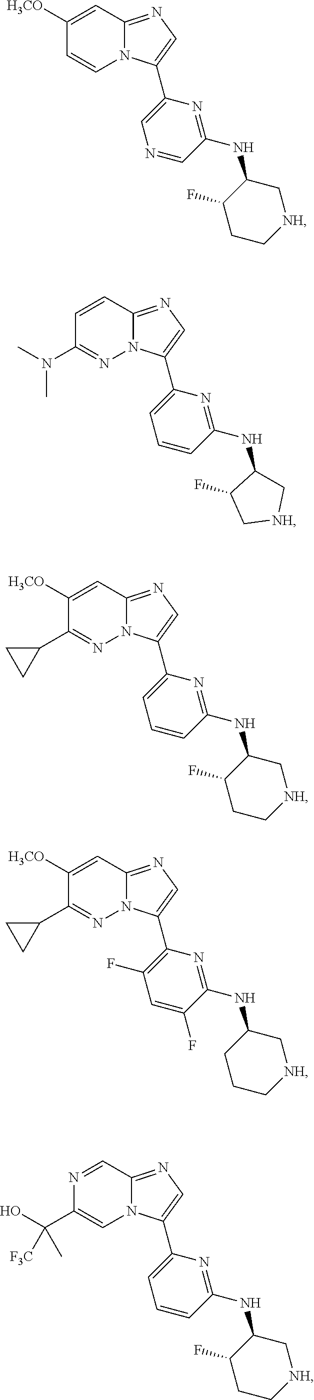 [SMILES: COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1 ; COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F]

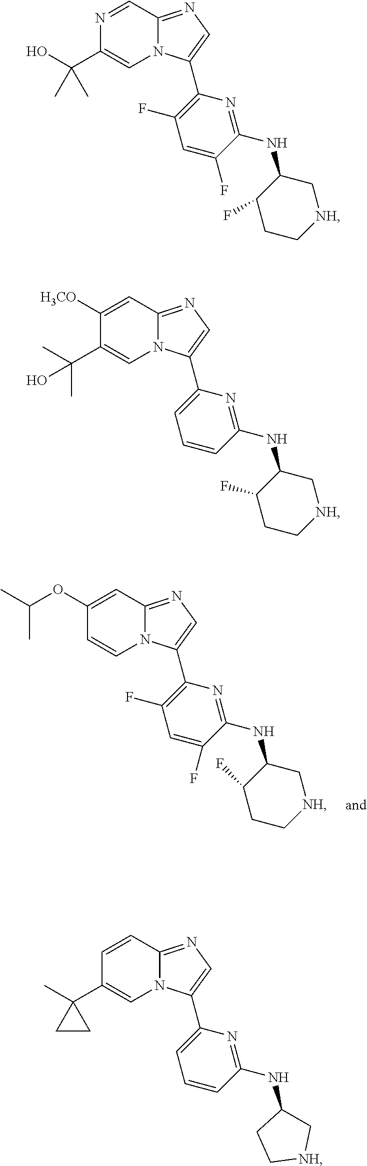 [SMILES: CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O ; CC(C)Oc1ccn2c(-c3nc(N[C@H]4CNCC[C@@H]4F)c(F)cc3F)cnc2c1 ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 126. A method of determining a compound that is effective at treating an inflammatory disease/disorder, acute myeloid leukemia (AML), or myelodysplastic syndrome (MDS), comprising:
determining from the IRAK1 IC₅₀ and IRAK4 IC₅₀ of the compound that the compound inhibits IRAK1 and IRAK4;
calculating from the IRAK1 and IRAK4 IC₅₀ of the compound that the compound has an IRAK4:IRAK1 potency ratio of less than about 40; and
administering the compound to a subject with an inflammatory disease/disorder, AML, or MDS, treating the inflammatory disease/disorder, AML, or MDS.

Clause 127. A method of determining a compound that is effective at treating an inflammatory disease/disorder, acute myeloid leukemia (AML), or myelodysplastic syndrome (MDS), comprising:
determining that the compound is an IRAK1/4 inhibitor which modifies the expression of one of more IRAK1/4-associated genes found to be differentially expressed in a subject with an inflammatory disease/disorder, AML, or MDS compared to a healthy control subject; and
administering the compound to the subject, treating the inflammatory disease/disorder, AML, or MDS.

Clause 128. The method of clause 127, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

Clause 129. The method of clause 127 or 128, wherein the compound decreases the expression of one or more IRAK1/4-associated genes selected from: KRAS, SOX5, HJMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 130. The method of any one of clauses 127 to 129, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 131. The method of any one of clauses 126 to 130, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

Clause 132. The method of any one of clauses 126 to 131, wherein the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AIL.

Clause 133. The method of any one of clauses 126 to 132, wherein the compound has an IRAK1 IC₅₀ of less than about 75 nM.

Clause 134. The method of any one of clauses 126 to 133, wherein the compound has an IRAK4 IC₅₀ of less than about 10 nM.

Clause 135. The method of any one of clauses 126 to 134, wherein the compound inhibits NF-kB-mediated signaling.

Clause 136. The method of clause 135, wherein inhibition of IRAK1 and IRAK4 inhibits NF-kB-mediated signaling.

Clause 137. The method of any one of clauses 126 to 136, wherein the compound inhibits TLR signaling and IL-1 signaling.

Clause 138. The method of clause 137, wherein the inhibition of RAK1 and IRAK4 inhibits TLR signaling and IL-1 signaling.

Clause 139. The method of any one of clauses 126 to 138, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 140. The method of clause 139, wherein the inhibition of RAK1 and IRAK4 inhibits cancer cell colony formation and/or cancer progenitor cell function.

Clause 141. The method of any one of clauses 126 to 140, wherein the compound provides sustained treatment of the inflammatory disease/disorder, AML, or MDS in the subject.

Clause 142. The method of clause 141, wherein the sustained treatment is greater than the treatment provided from a compound which inhibits IRAK1 without inhibiting IRAK4 or inhibits IRAK4 without inhibiting TRAK1.

Clause 143. The method of any one of clauses 126 to 142, wherein the compound continues to treat the inflammatory disease/disorder, AML, or MDS in the subject after the administration is stopped.

Clause 144. The method of clause 143, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 145. The method of any one of clauses 126 to 144, wherein the compound treats MDS or AML.

Clause 146. The method of any one of clauses 20 to 33, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (1-5010), (Ia-5010)-(Id-5010), (II-5010), (IIa-5010), (IIb-5010), (III-5010), (IlIa-5010), (IIb-5010), or a salt, ester, solvate, optical isomer, geometric isomer, or salt of an isomer of any one thereof.

Clause 147. The method of any one of clauses 126 to 146, wherein the compound is selected from:

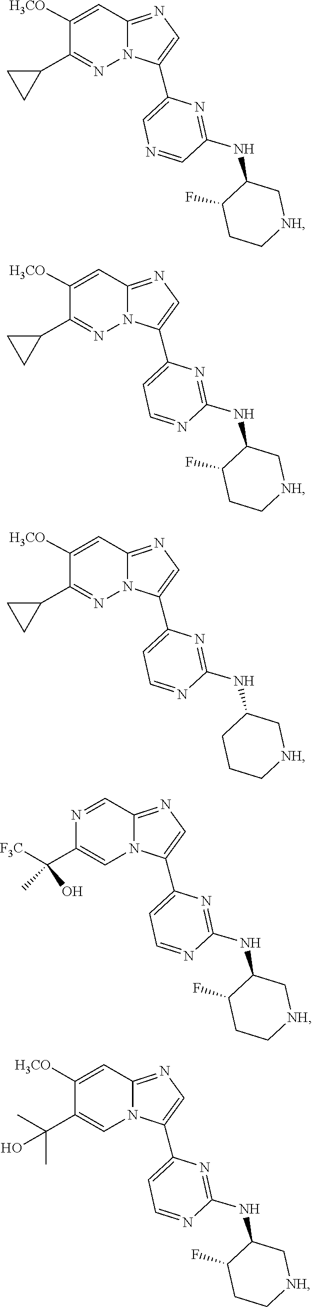 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F ; COc1cc2ncc(-c3ccnc(N[C@H]4CNCC[C@@H]4F)n3)n2cc1C(C)(C)O]

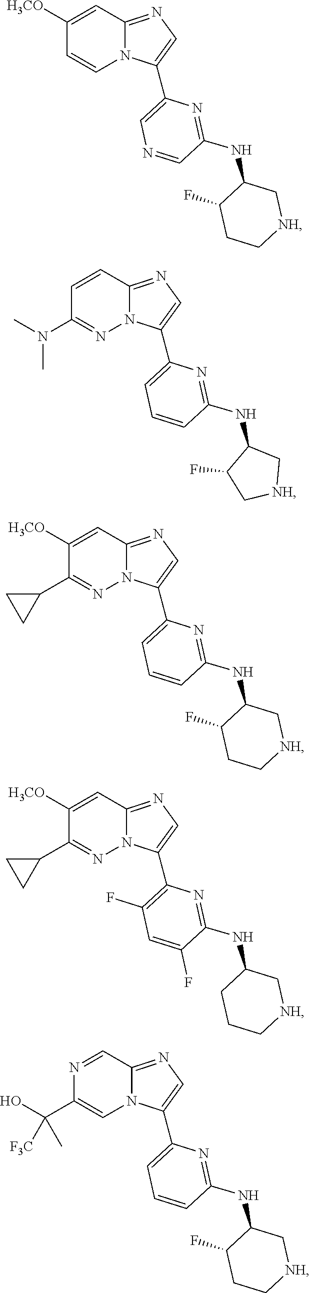 [SMILES: COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1 ; COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F]

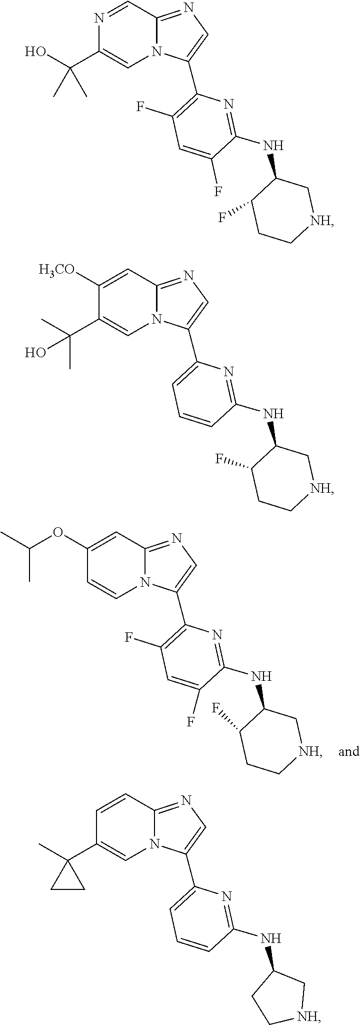 [SMILES: CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O ; CC(C)Oc1ccn2c(-c3nc(N[C@H]4CNCC[C@@H]4F)c(F)cc3F)cnc2c1 ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 148. A method of determining a subject with a disease/disorder that can be treated by the administration of a compound which inhibits IRAK1 and IRAK4, comprising:
determining that a subject with an inflammatory disease/disorder, acute myeloid leukemia (AML), or myelodysplastic syndrome (MDS) has elevated IRAK1 expression, IRAK4 expression, FLT3 expression, NF-kB signaling, TLR signaling, IL-1 signaling, or a combination thereof; and
administering to the subject a compound that inhibits IRAK1 and IRAK4 wherein the compound has an IRAK4:IRAK1 potency ratio of less than about 40, as calculated from the IRAK1 and IRAK4 IC₅₀ of the compound, thus treating the inflammatory disease/disorder, AML, or MDS.

Clause 149. The method of 148, wherein the subject has elevated IRAK1 expression, IRAK4 expression, or a combination thereof.

Clause 150. The method of clause 148 or 149, wherein the subject has elevated FLT3 expression.

Clause 151. A method of determining a subject with a disease/disorder that can be treated by the administration of a compound which inhibits IRAK1 and IRAK4, comprising:
determining that a subject with an inflammatory disease/disorder, acute myeloid leukemia (AML), or myelodysplastic syndrome (MDS) has elevated expression of one or more IRAK1/4-associated genes compared to a healthy control subject and/or decreased expression of one or more IRAK1/4-associated genes compared to a healthy control subject; and
administering to the subject a compound that inhibits IRAK1 and IRAK4, thus treating the inflammatory disease/disorder, AML, or MDS.

Clause 152. The method of clause 151, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

Clause 153. The method of clause 151 or 152, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 154. The method of any one of clauses 151 to 153, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 155. The method of any one of clauses 148 to 154, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

Clause 156. The method of any one of clauses 148 to 155, wherein the AML is relapsed AIL, refractory AIL, relapsed/refractory AML, AML with resistance to hypomethylating agents, AML with resistance to venetoclax, AML with resistance to hypomethylating agents and venetoclax, monocytic AMIL, or monocytic-like AML.

Clause 157. The method of any one of clauses 148 to 156, wherein the compound has an IRAK1 IC₅₀ of less than about 75 nM.

Clause 158. The method of any one of clauses 148 to 157, wherein the compound has an IRAK4 IC₅₀ of less than about 10 nM.

Clause 159. The method of any one of clauses 148 to 158, wherein the compound inhibits NF-kB-mediated signaling.

Clause 160. The method of clause 159, wherein inhibition of IRAK1 and IRAK4 inhibits NF-kB-mediated signaling.

Clause 161. The method of any one of clauses 148 to 160, wherein the compound inhibits TLR signaling and IL-1 signaling.

Clause 162. The method of clause 161, wherein the inhibition of IRAK1 and IRAK4 inhibits TLR signaling and IL-1 signaling.

Clause 163. The method of any one of clauses 148 to 162, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 164. The method of clause 163, wherein the inhibition of IRAK1 and IRAK4 inhibits cancer cell colony formation and/or cancer progenitor cell function.

Clause 165. The method of any one of clauses 148 to 164, wherein the compound provides sustained treatment of the inflammatory disease/disorder, AML, or MDS in the subject.

Clause 166. The method of clause 165, wherein the sustained treatment is greater than the treatment provided from a compound which inhibits IRAK1 without inhibiting IRAK4 or inhibits IRAK4 without inhibiting IRAK1.

Clause 167. The method of any one of clauses 148 to 166, wherein the compound continues to treat the inflammatory disease/disorder, AML, or MDS in the subject after the administration is stopped.

Clause 168. The method of clause 167, wherein the compound continues to treat the inflammatory disease/disorder, AML, or MDS in the subject for about 1 day, about 2 days, about 3 days, about 4 days, about 5 days, about 6 days, about one week, about two weeks, about three weeks, or about a month after the administration is stopped.

Clause 169. The method of any one of clauses 148 to 168, wherein the compound treats MDS or AML.

Clause 170. The method of any one of clauses 148 to 169, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (Ilf-5009)-(IIj-5009), (1-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

Clause 171. The method of any one of clauses 148 to 170, wherein the compound is selected from:

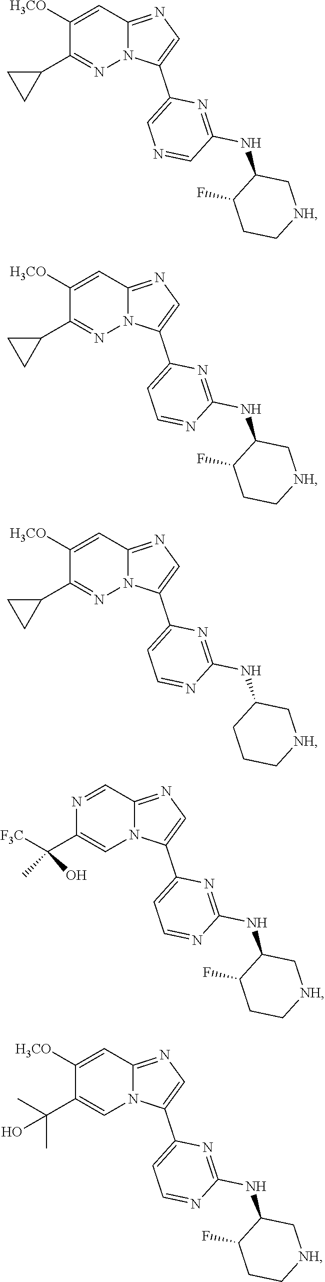 [SMILES: COc1cc2ncc(-c3cncc(N[C@H]4CNCC[C@@H]4F)n3)n2nc1C1CC1 ; COc1cc2ncc(-c3ccnc(N[C@H]4CNCC[C@@H]4F)n3)n2nc1C1CC1 ; COc1cc2ncc(-c3ccnc(N[C@H]4CCCNC4)n3)n2nc1C1CC1 ; C[C@@](O)(c1cn2c(-c3ccnc(N[C@H]4CNCC[C@@H]4F)n3)cnc2cn1)C(F)(F)F ; COc1cc2ncc(-c3ccnc(N[C@H]4CNCC[C@@H]4F)n3)n2cc1C(C)(C)O]

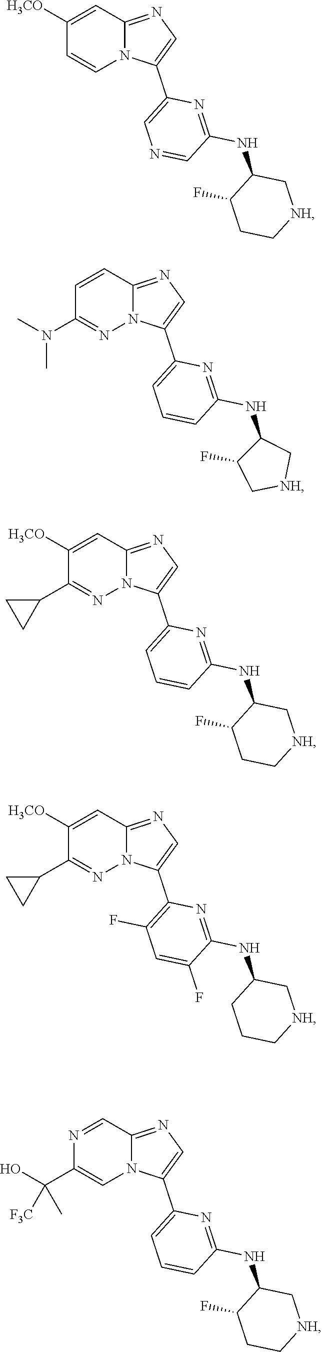 [SMILES: COc1ccn2c(-c3cncc(N[C@H]4CNCC[C@@H]4F)n3)cnc2c1 ; CN(C)c1ccc2ncc(-c3cccc(N[C@H]4CNC[C@@H]4F)n3)n2n1 ; COc1cc2ncc(-c3cccc(N[C@H]4CNCC[C@@H]4F)n3)n2nc1C1CC1 ; COc1cc2ncc(-c3nc(N[C@@H]4CCCNC4)c(F)cc3F)n2nc1C1CC1 ; CC(O)(c1cn2c(-c3cccc(N[C@H]4CNCC[C@@H]4F)n3)cnc2cn1)C(F)(F)F]

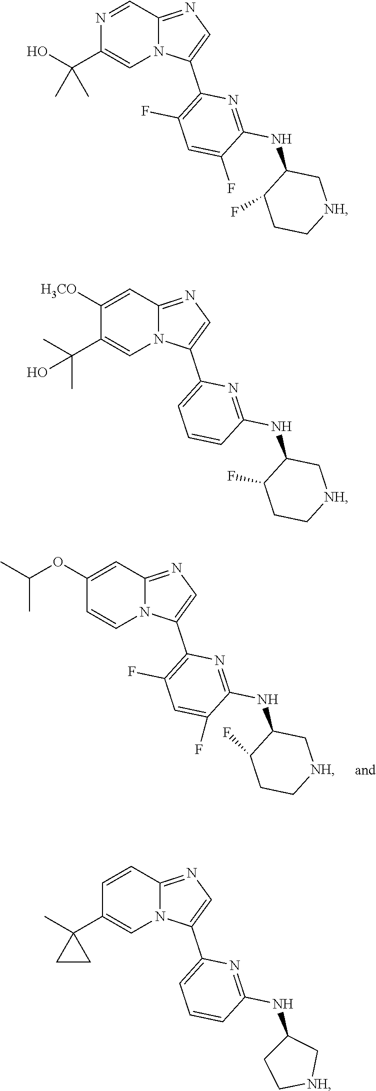 [SMILES: CC(C)(O)c1cn2c(-c3nc(N[C@H]4CNCC[C@@H]4F)c(F)cc3F)cnc2cn1 ; COc1cc2ncc(-c3cccc(N[C@H]4CNCC[C@@H]4F)n3)n2cc1C(C)(C)O ; CC(C)Oc1ccn2c(-c3nc(N[C@H]4CNCC[C@@H]4F)c(F)cc3F)cnc2c1 ; "and" ; CC1(c2ccc3ncc(-c4cccc(N[C@@H]5CCNC5)n4)n3c2)CC1]

or a salt, ester, solvate, optical isomer, geometric isomer, or salt of an isomer of any one thereof.

Clause 201. A method of treating an inflammatory disease/disorder, acute myeloid leukemia (AML), or myelodysplastic syndrome (MDS) in a subject in need thereof, the method comprising administering to the subject a compound that inhibits IRAK1 and IRAK4.

Clause 202. The method of clause 201, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

Clause 203. The method of clause 201, wherein the AIL is relapsed AML, refractory AML, relapsed/refractory AML, AML with resistance to hypomethylating agents, AML with resistance to venetoclax, AIL with resistance to hypomethylating agents and venetoclax, monocytic AML, or monocytic-like AML.

Clause 204. The method of clause 201, wherein the MDS is MDS with a splicing factor mutation, MDS with a mutation in isocitrate dehydrogenase 1, or MDS with a mutation in isocitrate dehydrogenase 2.

Clause 205. The method of clause 204, wherein the MDS with a splicing factor mutation comprises MDS with a splicing factor mutation in U2AF1, SRSF2, SF3B1, or ZRSR2.

Clause 206. The method of any one of clauses 201 to 205, wherein the subject in need thereof has elevated IRAK1 expression, IRAK4 expression, FLT3 expression, NF-kB signaling, TLR signaling, IL-1 signaling, or a combination thereof.

Clause 207. The method of clause 206, wherein the subject in need thereof has elevated IRAK1 expression, IRAK4 expression, or a combination thereof.

Clause 208. The method of clause 206 or 207, wherein the subject in need thereof has elevated FLT3 expression.

Clause 209. The method of any one of clauses 201 to 208, wherein the compound has an IRAK1 IC₅₀ of less than about 75 nM.

Clause 210. The method of any one of clauses 201 to 209, wherein the compound has an IRAK4 IC₅₀ of less than about 10 nM.

Clause 211. The method of any one of clauses 201 to 210, wherein the compound has IRAK4:IRAK1 potency ratio of less than about 40.

Clause 212. The method of any one of clauses 201 to 211, wherein the subject in need thereof has elevated expression of one or more IRAK1/4-associated genes compared to a healthy control subject and/or decreased expression of one or more IRAK1/4-associated genes compared to a healthy control subject.

Clause 213. The method of clause 212, wherein the subject in need thereof has elevated expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

Clause 214. The method of clause 212 or 213, wherein the subject in need thereof has decreased expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

Clause 215. The method of any one of clauses 212 to 214, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

Clause 216. The method of clause 215, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 217. The method of clause 215 or 216, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF1, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 218. The method of any one of clauses 201 to 217, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 219. The method of any one of clauses 201 to 218, wherein the compound provides sustained treatment of the inflammatory disease/disorder, AML, or MDS in the subject in need thereof.

Clause 220. The method of clause 219, wherein the sustained treatment is greater than the treatment provided from a compound which inhibits IRAK1 without inhibiting IRAK4 or inhibits IRAK4 without inhibiting IRAK1.

Clause 221. The method of any one of clauses 201 to 220, wherein the compound continues to treat the inflammatory disease/disorder, AML, or MDS in the subject in need thereof after the administration is stopped.

Clause 222. The method of clause 221, wherein the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped.

Clause 223a. The method of any one of clauses 201 to 222, wherein the compound is a compound of any one of Formulas (I-5007), (IIf-5007)-(IIk-5007), (IIm-5007)-(IIt-5007), (IIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (IIf-5009)-(IIj-5009), (1-5010), (Ia-5010)-(Id-5010), (1I-5010), (IIa-5010), (IIb-5010), (III-5010), (IIIa-5010), (IIIb-5010), or a salt, ester, solvate, optical isomer, geometric isomer, or salt of an isomer of any one thereof.

Clause 223b. The method of any one of clauses 201 to 223a, wherein the compound is selected from any one of Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

Clause 224. A method of determining effectiveness of a compound in treating an inflammatory disease/disorder, acute myeloid leukemia (AML), or myelodysplastic syndrome (MDS), comprising:
determining the compound's IRAK1 IC₅₀ and IRAK4 IC₅₀;
calculating an IRAK4:IRAK1 potency ratio from the IRAK1 IC₅₀ and the IRAK4 IC₅₀ of the compound;
wherein a potency ratio of less than about 40 is indicative of effectiveness of the compound in treating the inflammatory disease/disorder, AML, or MDS.

Clause 225. The method of clause 224, further comprising administering the compound to a subject with an inflammatory disease/disorder, AML, or MDS, treating the inflammatory disease/disorder, AML, or MDS.

Clause 226. A method of determining effectiveness of a compound in treating an inflammatory disease/disorder, acute myeloid leukemia (AML), or myelodysplastic syndrome (MDS), comprising:
determining that the compound is an IRAK1/4 inhibitor which modifies the expression of one of more IRAK1/4-associated genes found to be differentially expressed in a subject with an inflammatory disease/disorder, AML, or MDS compared to a healthy control subject.

Clause 227. The method of clause 226, further comprising administering the compound to the subject, treating the inflammatory disease/disorder, AML, or MDS.

Clause 228. The method of clause 226 or 227, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

Clause 229. The method of any one of clauses 226 to 228, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 230. The method of any one of clauses 226 to 229, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

Clause 231. The method of any one of clauses 224 to 230, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

Clause 232. The method of any one of clauses 224 to 230, wherein the AML is relapsed AML, refractory AIL, relapsed/refractory AML, AML with resistance to hypomethylating agents, AML with resistance to venetoclax, AML with resistance to hypomethylating agents and venetoclax, monocytic AMIL, or monocytic-like AML.

Clause 233. The method of any one of clauses 224 to 230, wherein the MDS is MDS with a splicing factor mutation, MDS with a mutation in isocitrate dehydrogenase 1, or MDS with a mutation in isocitrate dehydrogenase 2.

Clause 234. The method of clause 233, wherein the MDS with a splicing factor mutation comprises MDS with a splicing factor mutation in U2AF1, SRSF2, SF3B1, or ZRSR2.

Clause 235. The method of any one of clauses 224 to 234, wherein the compound has an IRAK1 IC₅₀ of less than about 75 nM.

Clause 236. The method of any one of clauses 224 to 235, wherein the compound has an IRAK4 IC₅₀ of less than about 10 nM.

Clause 237. The method of any one of clauses 224 to 236, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

Clause 238. The method of any one of clauses 224 to 237, wherein the compound provides sustained treatment of the inflammatory disease/disorder, AML, or MDS when administered to a subject in need thereof.

Clause 239. The method of clause 238, wherein the sustained treatment is greater than the treatment provided from a compound which inhibits IRAK1 without inhibiting IRAK4 or inhibits IRAK4 without inhibiting IRAK1.

Clause 240. The method of any one of clauses 224 to 239, wherein the compound continues to treat the inflammatory disease/disorder, AML, or MDS in a subject in need thereof after administration of the compound to the subject is stopped.

Clause 241. The method of clause 240, wherein the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped.

Clause 242a. The method of any one of clauses 224 to 241, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (1-5009), (IIf-5009)-(IIj-5009), (1-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

Clause 242b. The method of any one of clauses 224 to 242a, wherein the compound is selected from any one of Compounds 1-7 to 295-7, 1a-7 to 79a-7, 1-3 to 68-3, 1-8 to 137-8, 1a-8 to 84a-8, 1-9 to 138-9, and 1-10 to 84-10, or a salt, ester, solvate, optical isomer, geometric isomer, or salt of an isomer of any one thereof.

### EXAMPLES

### Example 1: Inhibition of Both IRAK1 and IRAK4 is Required for Complete Suppression of NF-κB Signaling Across Multiple Receptor-Mediated Pathways in MDS and AML

In this report, a pharmacological approach was used in which a series of IRAK inhibitors of varying relative potency at IRAK4:IRAK1 were examined for both inhibition of NF-kB activation in AML cells and inhibition of leukemic progenitor cell function in vitro. It was found that potency and efficacy for antagonism at NF-kB requires effective inhibition of both IRAK1 and IRAK4. It was also found that the relative potency of NF-kB inhibition correlates with suppression of leukemic progenitor cell function in vitro, providing pharmacological validation that inhibition of both IRAK1 and IRAK4 are necessary for optimal inhibition of NF-kB and effect on leukemia progenitor cell function.

An NF-kB reporter system expressed in human AML cells (THP1) was used to measure NF-kB dependent activation in response to a variety of IRAK4, IRAK1, or IRAK1/4 antagonists. The cells are highly responsive to both TLR agonists as well as to IL-1β, which allows for measurement of IRAK-mediated antagonism of multiple receptor-mediated pathways. Using the IRAK4-selective antagonists PF-06650833 and BAY 1834845 it was found that both compounds fully suppress signaling through TLR2 (IC₅₀ vs. Pam3CSK4=7.2 and 150 nM, respectively). The IRAK4 antagonists inhibit signaling through the IL-JR with similar relative potency (IC₅₀ vs. IL-1β=5.7 and 81 nM), but do not fully suppress signaling through this receptor (Span=75% and 59%, respectively). Interestingly, the IRAK1 selective covalent inhibitor JH-X-119-01 does not inhibit signaling against either the TLR or the IL-1 receptor agonist. Together these data imply that neither IRAK4 nor IRAK1 inhibition alone is sufficient to fully inhibit NF-kB-mediated signaling through multiple receptor mediated pathways.

For this reason, the activity of a series of IRAK4/IRAK1 inhibitors was examined in AML cells. This allowed for a study of the effect of adding in additional inhibitory activity at IRAK1 on the background of high potency IRAK4 inhibitors. Using two reference compounds with varying IRAK4:IRAK1 potency, the relative ability of these two compounds to inhibit TLR-agonist or IL-1β-agonist stimulated NF-kB activity in AML was compared. Compound 15 has an IRAK4:IRAK1 potency ratio of 36 whereas Compound 14 has an IRAK4:IRAK1 potency ratio of 100. Unlike what was observed with the IRAK4-selective antagonists, both compounds can completely suppress NF-kB signaling through IL-IP, with relative potencies apparently determined by their activity at IRAK1: (IC₅₀ vs. Pam3CSK4=6.3 and 73.8 nM for Compound 15 and Compound 14, respectively); (IC₅₀ vs. IL-1β=9.3 and 227 nM for Compound 15 and Compound 14, respectively).

Finally, the correlation between potency in the biochemical kinase assay at IRAK1 or IRAK4, activity in the NF-kB reporter assay, and leukemic progenitor cell activity in the colony forming assay was examined for a series of compounds. A correlation was found between kinase activity for both IRAK1 and IRAK4 and NF-kB activity that extends to the leukemia colony forming assays. This suggests that NF-kB signaling contributes to leukemia progenitor cell function and that optimal inhibition requires potent antagonism of both IRAK1 and IRAK4 in the setting of MDS and AML.

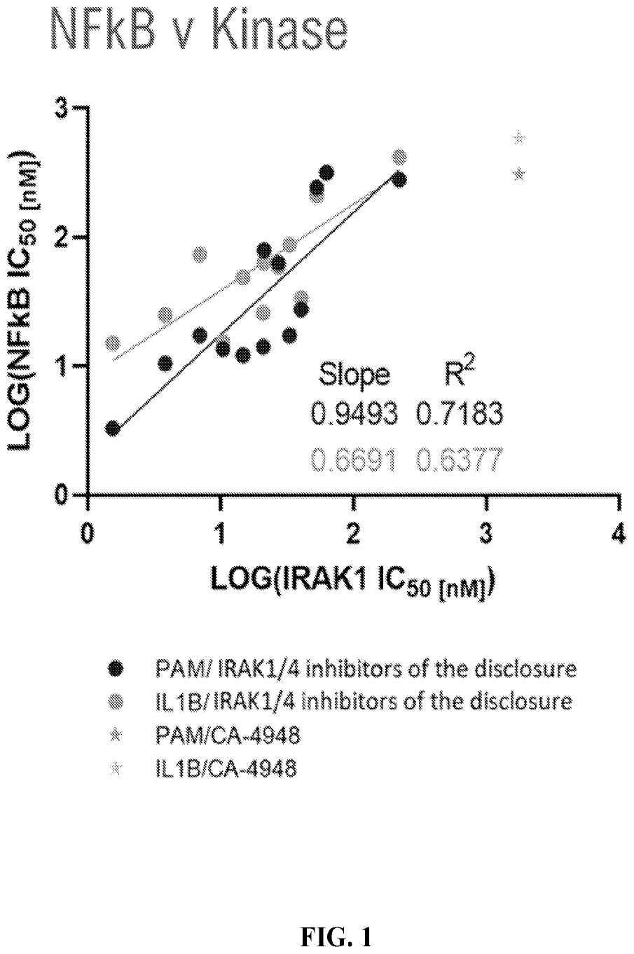

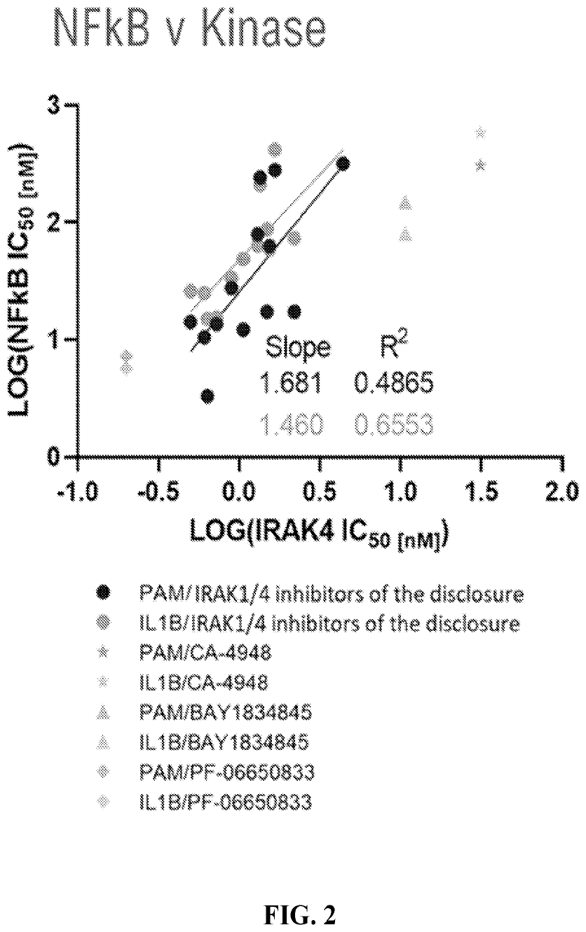

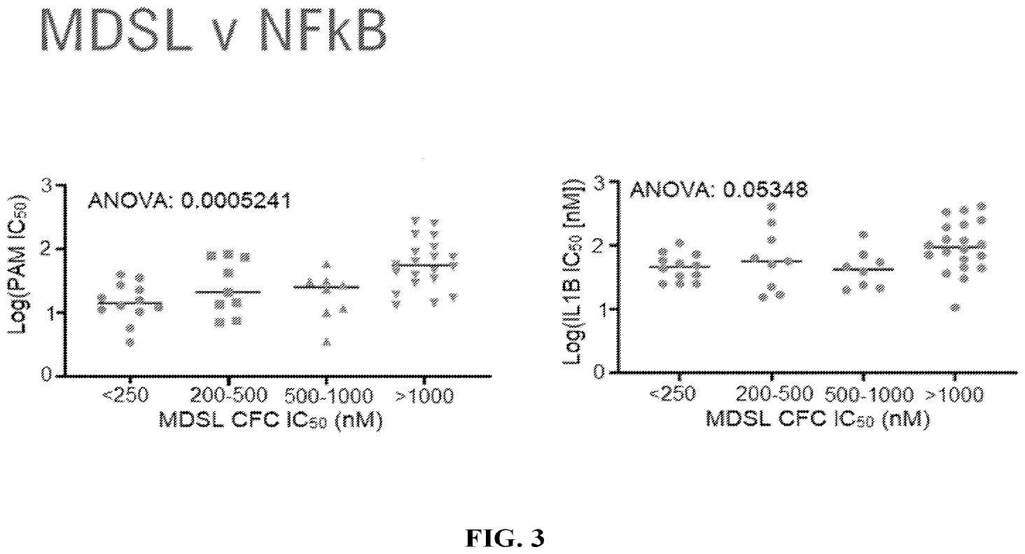

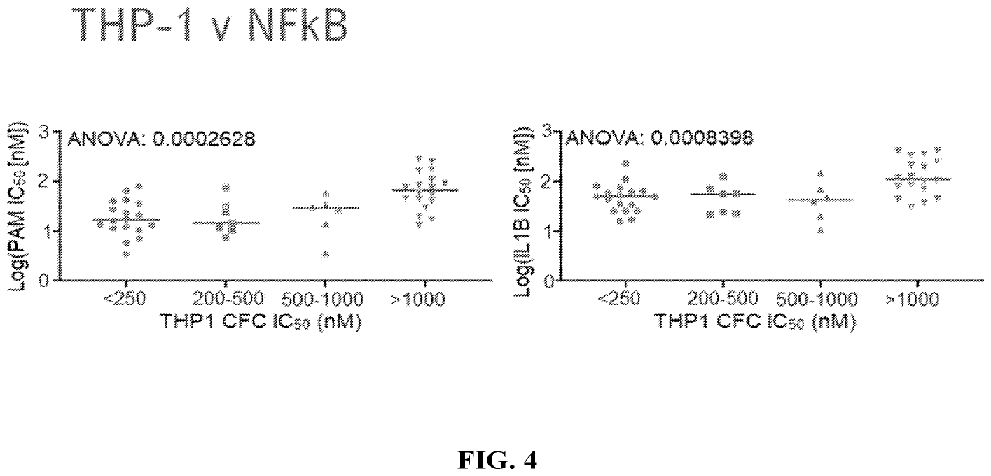

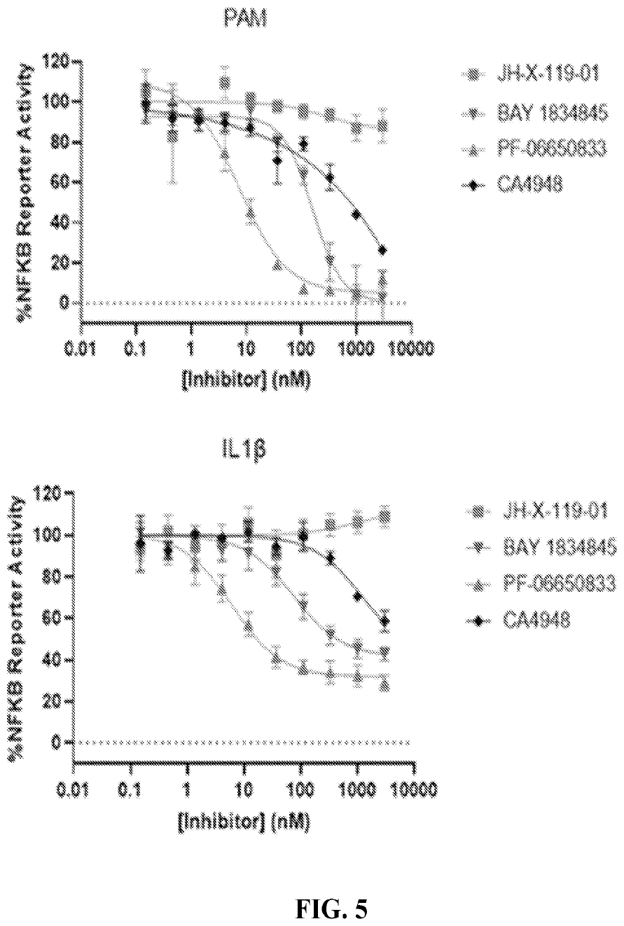

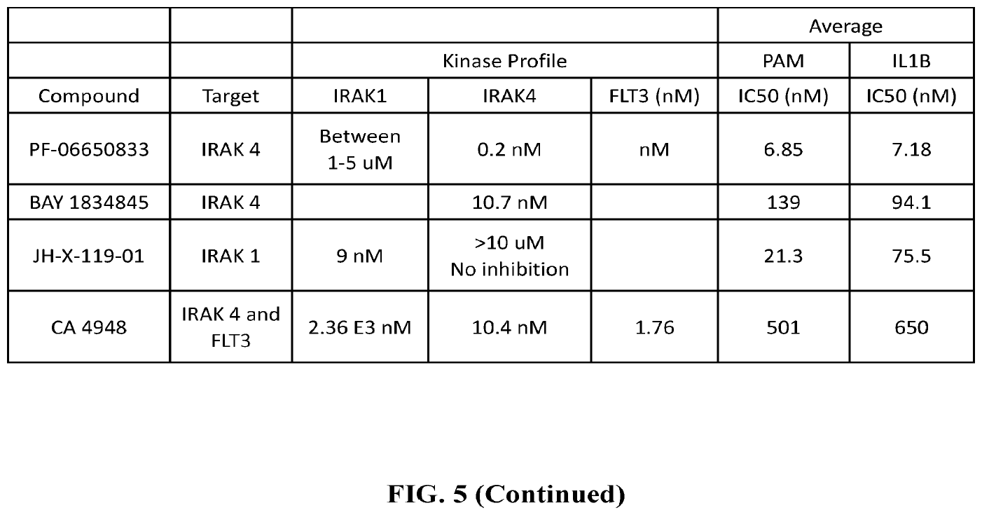

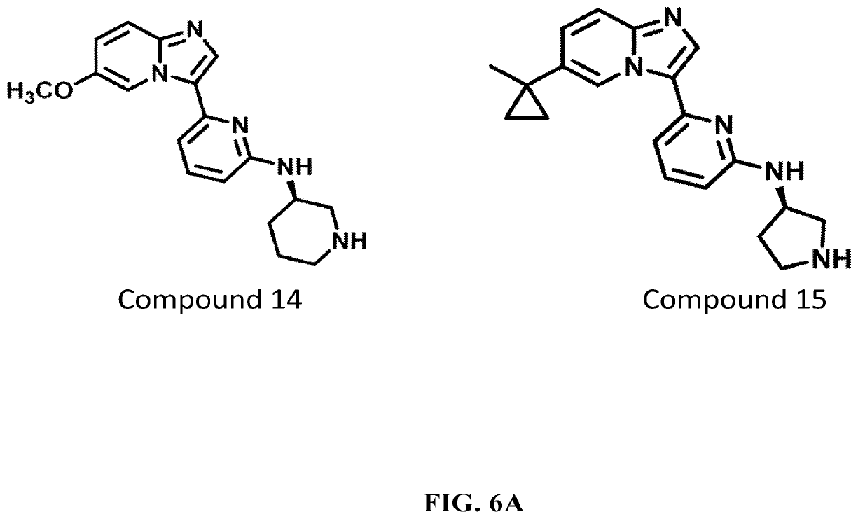

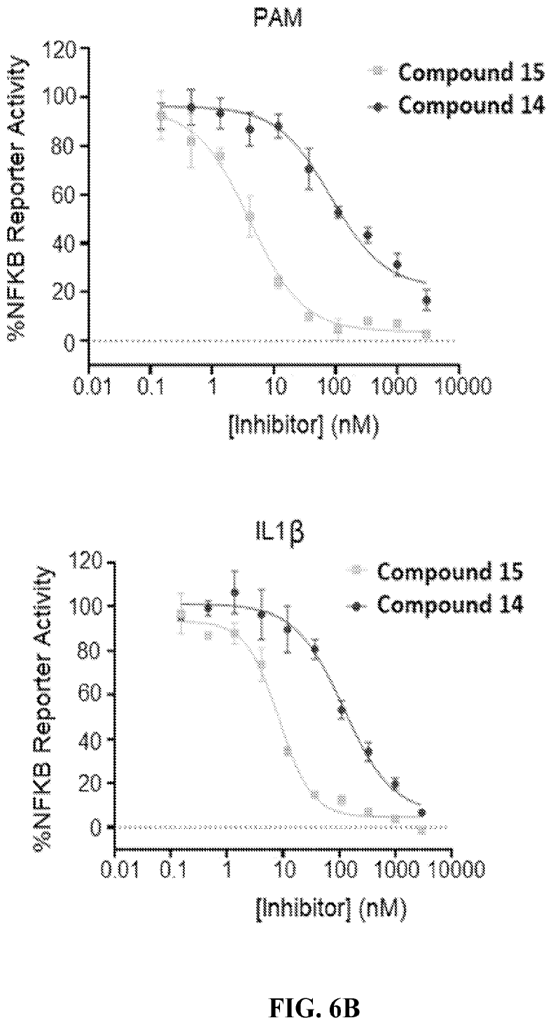

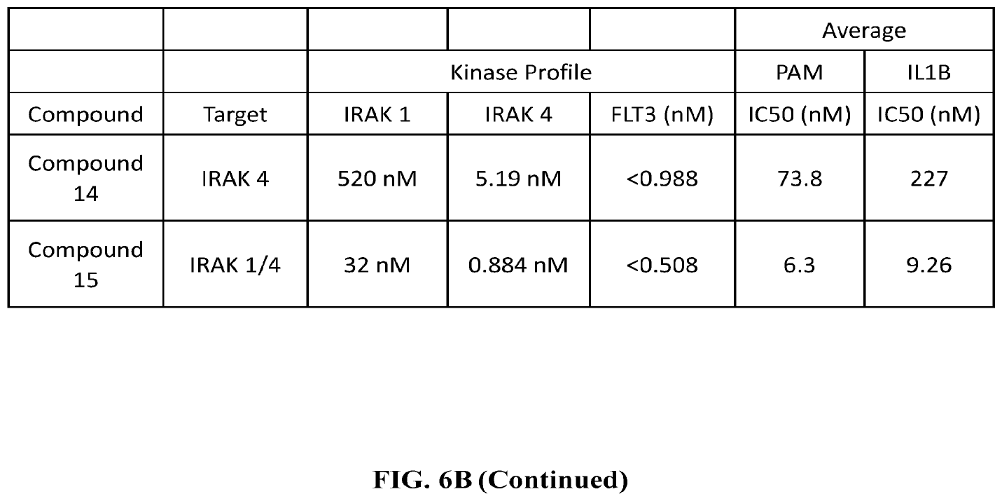

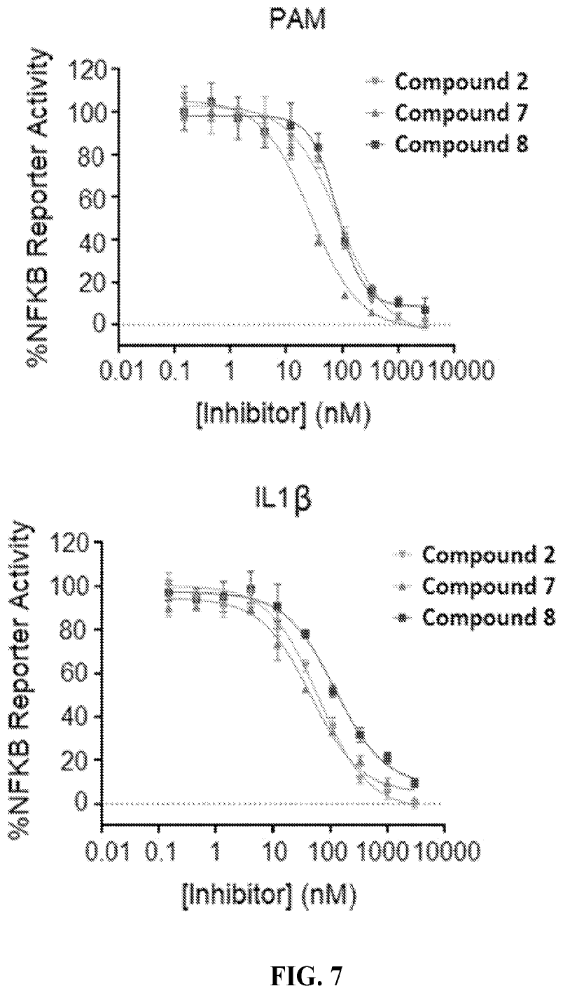

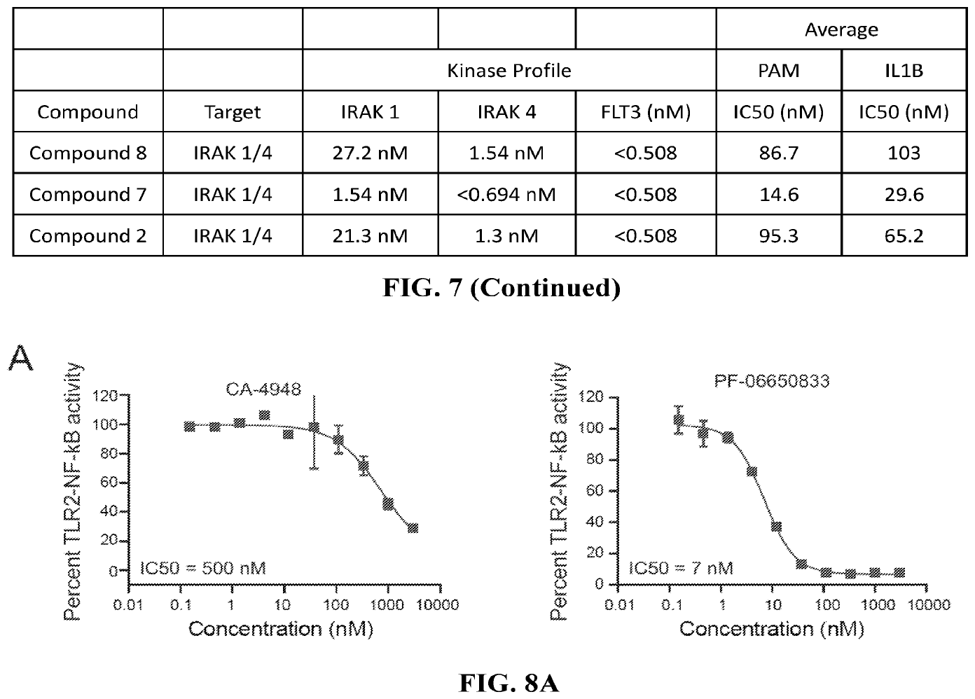

See FIGS. 1-7 and Table 1 for the data described in Example 1.

TABLE 1

| | Reaction Bio | | THP-1 NFkB | | | | | |
|---|---|---|---|---|---|---|---|---|
| | IRAK1 | IRAK4 | PAM | IL1B | LOG | LOG | LOG | LOG |
| Compound | IC50 | IC50 | IC50 | IC50 | (IRAK1) | (IRAK4) | (PAM) | (IL1B) |
| 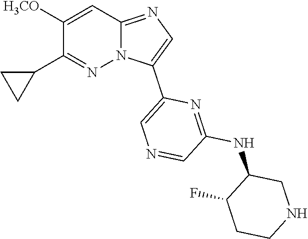 [SMILES: COc1cc2ncc(-c3cncc(N[C@H]4CNCC[C@@H]4F)n3)n2nc1C1CC1] | 52.8 | 1.350 | 242 | 209 | 1.72 | 0.13 | 2.38 | 2.32 |
| Compound 1 | | | | | | | | |
| 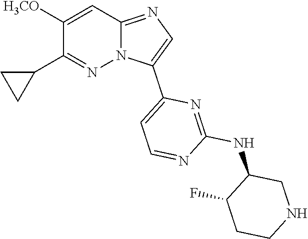 [SMILES: COc1cc2ncc(-c3ccnc(N[C@H]4CNCC[C@@H]4F)n3)n2nc1C1CC1] | 21.3 | 1.300 | 79.2 | 63.5 | 1.33 | 0.11 | 1.90 | 1.80 |
| Compound 2 | | | | | | | | |
| 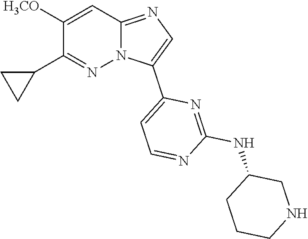 [SMILES: COc1cc2ncc(-c3ccnc(N[C@H]4CCCNC4)n3)n2nc1C1CC1] | 62.6 | 4.400 | 317 | 316 | 1.80 | 0.64 | 2.50 | 2.50 |
| Compound 3 | | | | | | | | |
| 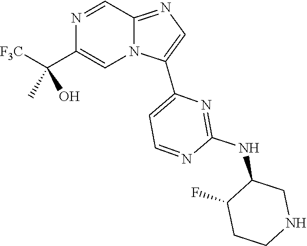 [SMILES: C[C@@](O)(c1cn2c(-c3ccnc(N[C@H]4CNCC[C@@H]4F)n3)cnc2cn1)C(F)(F)F] | 6.96 | 2.200 | 17.3 | 73.3 | 0.84 | 0.34 | 1.24 | 1.87 |
| Compound 4 | | | | | | | | |
| 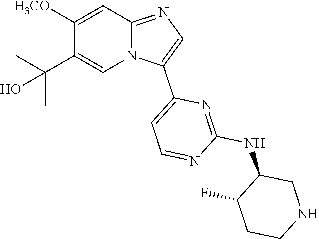 [SMILES: COc1cc2ncc(-c3ccnc(N[C@H]4CNCC[C@@H]4F)n3)n2cc1C(C)(C)O] | 3.84 | 0.609 | 10.5 | 25 | 0.58 | −0.22 | 1.02 | 1.40 |
| Compound 5 | | | | | | | | |
| 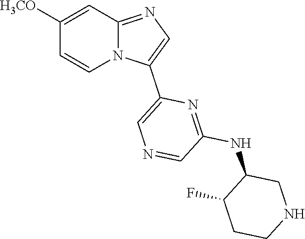 [SMILES: COc1ccn2c(-c3cncc(N[C@H]4CNCC[C@@H]4F)n3)cnc2c1] | 40.4 | 0.898 | 27.6 | 33.6 | 1.61 | −0.05 | 1.44 | 1.53 |
| Compound 6 | | | | | | | | |
| 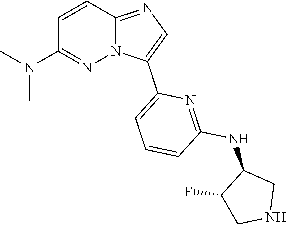 [SMILES: CN(C)c1ccc2ncc(-c3cccc(N[C@H]4CNC[C@@H]4F)n3)n2n1] | 1.54 | 0.637 | 3.3 | 15.1 | 0.19 | −0.20 | 0.52 | 1.18 |
| Compound 7 | | | | | | | | |
| 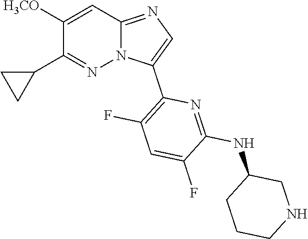 [SMILES: COc1cc2ncc(-c3nc(N[C@@H]4CCCNC4)c(F)cc3F)n2nc1C1CC1] | 27.2 | 1.540 | 62.9 | 58.7 | 1.43 | 0.19 | 1.80 | 1.77 |
| Compound 8 | | | | | | | | |
| 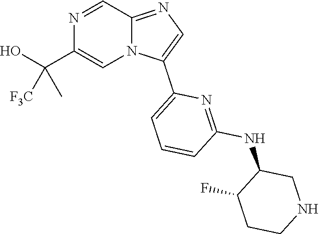 [SMILES: CC(O)(c1cn2c(-c3cccc(N[C@H]4CNCC[C@@H]4F)n3)cnc2cn1)C(F)(F)F] | 21 | 0.500 | 14.2 | 25.9 | 1.32 | −0.30 | 1.15 | 1.41 |
| Compound 9 | | | | | | | | |
| 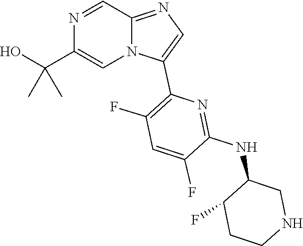 [SMILES: CC(C)(O)c1cn2c(-c3nc(N[C@H]4CNCC[C@@H]4F)c(F)cc3F)cnc2cn1] | 33 | 1.490 | 17.3 | 87.5 | 1.52 | 0.17 | 1.24 | 1.94 |
| Compound 10 | | | | | | | | |
| 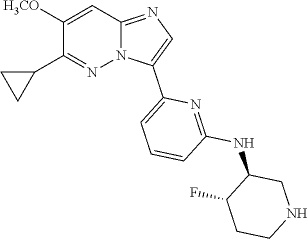 [SMILES: COc1cc2ncc(-c3cccc(N[C@H]4CNCC[C@@H]4F)n3)n2nc1C1CC1] | 220 | 1.670 | 280 | 419 | 2.34 | 0.22 | 2.45 | 2.62 |
| Compound 11 | | | | | | | | |
| 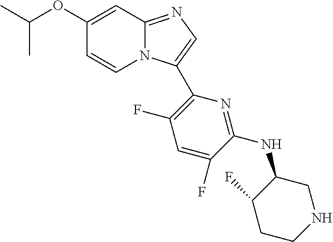 [SMILES: CC(C)Oc1ccn2c(-c3nc(N[C@H]4CNCC[C@@H]4F)c(F)cc3F)cnc2c1] | 14.7 | 1.060 | 12.2 | 48.8 | 1.17 | 0.03 | 1.09 | 1.69 |
| Compound 12 | | | | | | | | |
| 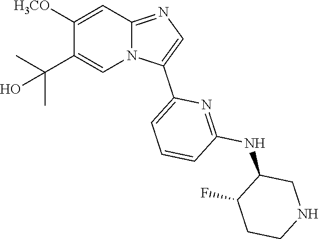 [SMILES: COc1cc2ncc(-c3cccc(N[C@H]4CNCC[C@@H]4F)n3)n2cc1C(C)(C)O] | 10.4 | 0.727 | 13.6 | 15.4 | 1.02 | −0.14 | 1.13 | 1.19 |
| Compound 13 | | | | | | | | |
| CA-4948 (Emavusertib) | 1757 | 31.2 | 307 | 580 | 3.24 | 1.49 | 2.49 | 2.76 |
| BAY1834845 | NA | 10.7 | 150 | 81.2 | | 1.02938378 | 2.18 | 1.91 |
| PF-06650833 | NA | 0.2 | 496 | 678 | | −0.69897 | 2.70 | 2.83 |

### Example 2: IRAK1 Contributes to IRAK4 Inhibitor Resistance Via Non-Canonical Signaling Mechanisms in MDS/AML

Herein, the mechanisms of resistance to IRAK4-selective inhibitors were explored in MDS/AML. The findings uncovered non-canonical IRAK1/4 signaling paradigms driving leukemic stem and progenitor cells (LSPC) and yielded novel therapeutic strategies for MDS/AML.

Consistent with the initial observations from the clinical trials, inhibition or deletion of IRAK4 in a panel of MDS/AML cell lines and patient samples resulted in incomplete suppression of LSPCs and a corresponding activation of innate immune pathways. Given the evolutionary conserved redundancy of IRAK-dependent pathways, the expression of IRAK paralogs was first examined. In IRAK4 knockout (KO) MDS/AML cells, only overexpression and activation of the IRAK4 paralog, IRAK1 was observed. IRAK4 kinase inhibitors or PROTACs resulted in a similar compensatory activation of IRAK1 in MDS/AML. To validate the compensation of IRAK1, IRAK1 was deleted in MDS/AML cells and it was found that concomitant suppression of IRAK1 led to a significant reduction of LSPC function in IRAK4-KO MDS/AML cells. Co-deletion of RAK1 and IRAK4 similarly reduced leukemic engraftment and extended survival in an AML xenograft model. Hence, IRAK1 mitigates the efficacy of IRAK4-inhibitors in MDS/AML.

Canonical IRAK signaling depends on recruitment of MyD88 and IRAK4 to activated receptors, which results in the subsequent recruitment and activation of IRAK1. Based on this canonical model, IRAK1 and IRAK4 are thus independently essential for signaling. Since dual inhibition of IRAK1/4 is obligatory for suppression of LSPCs, it was posited that conventional IRAK signaling is not sufficient nor essential in MDS/AML. Therefore, the role of proximal upstream (MyD88) and downstream (TRAF6) effectors of IRAK1/4 was first examined. Deletion of TRAF6 resulted in suppression of LSPCs as observed upon inhibition of IRAK1/4. In contrast, MyD88-KO AML cells did not exhibit a functional defect but did retain sensitivity to deletion of IRAK1 and IRAK4, indicating canonical MyD88-dependent signaling is not operational in MDS/AML. To investigate non-canonical IRAK1/4 signaling RNA- and ATAC-seq was performed and identified gene expression programs dependent on IRAK1 and/or IRAK4, yet independent of MyD88. IRAK1/4 deletion correlated with dysregulation of transcription factors involved in stem cell maintenance and myeloid differentiation. The requirement for IRAK1/4 in preserving an undifferentiated LSPC state was corroborated by morphological assessment of IRAK1/4-KO MDS/AML cells. To delineate the signaling mechanisms by which IRAK1/4 governs stem programs, mass spectrometry proteomics was performed which identified unique IRAK4- and IRAK1-interacting proteins. Integration of the proteomic and transcriptomic studies identified pathways (HIF1A, STAT3/IRFs, E2F4) implicated in stem cell and undifferentiated states as effectors of IRAK1 and IRAK4.

To extend these studies to improve the efficacy of IRAK4-targeted therapy in MDS/AML, a novel series of structurally related inhibitors were developed targeting IRAK1 and IRAK4 (Compound 15: IRAK1 IC₅₀=32 nM; IRAK4 IC₅₀=0.9 nM) or IRAK4 alone (Compound 14: IRAK1 IC₅₀=>500 nM; IRAK4 IC₅₀=5 nM). In human AML cell lines and patient samples, the dual IRAK1/4 inhibitor was significantly more effective at inducing cell death and differentiation, and attenuating LSPC function as compared to the IRAK4 inhibitor. Thus, the mandate for targeting both IRAK1 and IRAK4 established in these genetic studies translates to pharmacologic interventions.

Overall, it was demonstrated that compensation by IRAK1 is a barrier to IRAK4-directed therapy and revealed that dual IRAK1/4 inhibitors are needed for achieving optimal clinical response in MDS/AML. In the process, novel and non-canonical signaling paradigms governing the oncogenic role of IRAK1/4 were also uncovered.

### Example 3: IRAK1 and IRAK4 are Required for Leukemic Stem and Progenitor Cells Via Myd88-Independent Mechanisms in Myeloid Malignancies

Dysregulation of innate immune signaling is a hallmark of hematologic malignancies. Recent therapeutic efforts to subvert aberrant innate immune signaling in MDS and AML have focused on the kinase IRAK4. IRAK4 inhibitors have achieved promising, though moderate, responses in pre-clinical studies and in clinical trials for MDS and AML. The reasons underlying the limited responses to IRAK4 inhibitors remain unknown. Herein, it was revealed that inhibiting IRAK4 in leukemic cells elicits functional complementation and compensation by its paralog, IRAK1. Using genetic approaches, it was demonstrated that co-targeting IRAK1 and IRAK4 is required to suppress leukemic stem/progenitor cell (LSPC) function and induce differentiation in cell lines and patient-derived cells. While IRAK1 and IRAK4 are presumed to require the adaptor MyD88 for signaling, it was found that complimentary and compensatory IRAK1 and IRAK4 dependencies in MDS/AML occur via non-canonical MyD88-independent pathways. Genomic and proteomic analyses revealed that IRAK1 and IRAK4 preserve the undifferentiated state of MDS/AML LSPCs by coordinating a network of pathways, including ones that converge on the PRC2 complex and interferon signaling. To translate these findings, a structure-based design of a potent and selective dual IRAK1 and IRAK4 inhibitor, Compound 15, was implemented. MDS/AML cell lines and patient-derived samples showed a significant suppression of LSPC function and viability when treated with Compound 15 as compared to selective IRAK4 inhibitors. The results provide a mechanistic basis and rationale for co-targeting IRAK1 and IRAK4 for the treatment of cancers, including MDS/AML.

### Material and Methods

### Compounds and Materials

PF-06650833 (PZ0327-5MG) were Sigma-Aldrich. CA-4948 and IRAK4 degrader-1 was purchased from ChemExpress. Compound 14 and Compound 15 were obtained from Kurome Therapeutics.

### Kinome Screens

Dissociation constants (Ka) were measured at DiscoverX using the KINOMEscan Profiling Service. Kinase inhibition (IC₅₀) was measured at Reaction Biology using the Kinase Assay service.

### Cell Lines and Patient-Derived Samples

THP1, OCI-AML3, and TF1 were purchased from the American Type Culture Collection. MDSL cells were provided by K. Tohyama (Kawasaki Medical School, Okayama, Japan). THP1 were cultured in RPMI-1640 medium with 10% FBS and 1% penicillin-streptomycin. MDSL and TF1 cell lines were cultured with complete RPMI supplemented with 10 ng ml⁻¹ recombinant human IL-3 (Stem Cell Technologies). OCIAML3 were cultured with Dulbecco's Modified Eagle Medium (DMEM) with 10% FBS and 1% penicillin-streptomycin. Bone marrow (BM) and peripheral blood samples from patients with AML at initial diagnosis were obtained with written informed consent and approved by the institutional review board of Cincinnati Children's Hospital Medical Center and University of Cincinnati, or from the Eastern Cooperative Oncology Group (ECOG). These samples had been obtained within the framework of routine diagnostic BM aspirations after written informed consent in accordance with the Declaration of Helsinki. De-identified leukemic cells from peripheral blood and BM of patients with AML were obtained at CCHMC following consent under the IRB approved Study ID #2008-0021. To obtain sufficient cell numbers for functional studies, the AML samples were first expanded in immunocompromised mice. Patient samples were RBC lysed and coated with OKT3 antibody (UCHT1, BioXCell). Primary NSGS mice were given a single 30 mg/kg intraperitoneal dose of busulfan 24 hrs prior to intravenous or intrafemoral injection of the OKT3-coated cell preparations. After −60 days (median 56 days, average 70 days) in xenografted mice, single cell spleen preparations were isolated and cultured in IMDM, 20% FBS, and 10 ng/mL of cytokines (SCF, TPO, FLT3L, IL3, IL-6). AML samples capable of indefinite expansion in liquid culture were selected for use in this study: AML(1714) and AML(1294).

### Generation of CRISPR/Cas9 Mutant Cells

The THP1 IRAK4^{KO} clone was generated from a pre-derived WT THP1 clone. To generate the THP1 IRAK1^{KO} and IRAK1/4^{dKO}, the WT and IRAK4^{KO} clone were suspended in buffer R with Cas9-NLS and a modified synthetic gRNA targeting exon 1 of IRAK1 (Synthego) and electroporated (1700 mV×20 ms×1 pulse) using the Neon Transfection system (Invitrogen). Transfected cells were recovered for 48 hours in antibiotic-free RPMI-1640 with 1% FBS. Following recovery, transfected cells were plated in 96 well plates at a target density of 0.25 cells/well to isolate single clones. Clones were expanded and screened for IRAK1 deletion by immunoblotting. Deletion was confirmed by PCR amplification of the PAM site for Sanger sequencing. MDSL, TF-1, OCIAML3, AML(1714) and AIL(1294) IRAK4^{KO} clones were generated from parental populations following the protocol outlined above using a synthetic gRNA targeting Exon 1 of IRAK4. WT clones were derived in parallel by plating parental cells in single-cell suspensions and expanding clones. MDSL and THP1 MyD88^{KO} and TRAF6^{KO} clones were generated from parental populations following the protocol outlined above using a synthetic gRNA targeting Exon 1 of MyD88 and Exon 1 of TRAF6, respectively.

### Clonogenic Assays

Clonogenic progenitor frequencies were determined by plating cell lines or patient samples in Methocult H4434 (Stem Cell Technologies) at a density of 500 cells per ml in SmartDish meniscus-free 6-well plates. Plates were kept in humidified chambers and colonies were imaged and manually scored after 9-14 days using the STEMvision counter (Stem Cell). For inhibitor treatments, cells were plated in methylcellulose (StemCell technologies H4236) at the indicated concentrations of inhibitors. Cells were incubated at 37° C. and 5% C₀₂. Colonies were scored after ˜10 days in culture.

### Xenografts

Animals were bred and housed in an Association for Assessment and Accreditation of Laboratory Animal Care-accredited animal facility. WT, IRAK1^{KO} IRAK4^{KO} and IRAK1/4^{dKO} THP1 cells were suspended in PBS and injected via tail vein into NOD-scid IL2Rgnull-3/GM/SF (NSGS) mice at a dose of 2.5×10⁵ cells per mouse. Moribund mice were sacrificed and assessed for leukemic burden measurements. Briefly, mice were euthanized with carbon dioxide following the AVMA Guidelines for the Euthanasia of Animals and BM cells were immediately extracted by breaking the femurs with a mortar and pestle. BM cells were frozen in FBS with 10% DMSO until time of analysis. BM was analyzed for huCD45 (BDPharmingen, 555485) and huCD33 (BDPharmingen, 555450) expression by flow cytometry using a BD LSRFortessa (BD Biosciences). For staining, 1×10⁶ cells from each BM sample were incubated with antibodies diluted 1:100 in a solution of PBS, 0.2% FBS for 30 minutes on ice in the dark. Cells were washed once with PBS, resuspended in PBS with 0.2% FBS, and immediately analyzed by flow cytometry. Spleens and livers from sacrificed were photographed and preserved in 10% phosphate buffered formalin for histological assessment of leukemic infiltration. For survival analysis, time of death was recorded, and Kaplan Meier survival analysis was performed using GraphPad Prism version 9 for Mac (GraphPad Software).

### Immunoblotting

Protein lysates were made by lysing cells in cold RIPA lysis buffer (50 mM Tris-HCl, 150 mM NaCl, 1 mM ethylenediaminetetraacetic acid (EDTA), 1% Triton X-100, and 0.1% sodium dodecyl sulfate (SDS)) in the presence of sodium orthovanadate, phenylmethylsulfonyl fluoride (PMSF), and protease and phosphatase inhibitors. Protein concentration was quantified using bicinchoninic acid (BCA) assay (Pierce, Cat #23225). Protein lysates were separated by SDS-polyacrylamide gel electrophoresis (BIO-RAD), transferred to nitrocellulose membranes (BIO-RAD, Cat #1620112), and immunoblotted. The following antibodies were used for western blot analysis: GAPDH (Cell Signaling, Cat #D16H11, 1:1000 milk), Vinculin (Cell Signaling, Cat #13901, 1:1000 BSA), IRAK4 (Cell Signaling, Cat #4363, 1:1000 BSA), IRAK1 (Santa Cruz, Cat #sc-5288, 1:1000 milk), phospho-IRAK1 (T209) (Assay Biotech, Cat #A1074, 1:500 BSA), phospho-SAPK/JNK (Thr183/Tyr185) (Cell Signaling, Cat #4668, 1:500 BSA), phospho-p38 MAPK (Thr180/Tyr182) (Cell Signaling, Cat #4631, 1:500 BSA), phospho-p44/42 MAPK (ERK1/2. Thr202/Tyr204) (Cell signaling, Cat #4377, 1:1000 BSA), phospho-IKKa/b (Serl76/180) (Cell Signaling, Cat #2697, 1:500 BSA), MyD88 (Cell Signaling, Cat #4283, 1:1000 BSA, TRAF6 (Santa Cruz, Cat #sc-7221, 1:1000 milk), IRAK2 (Cell Signaling, Cat #4367, 1:1000 BSA), peroxidase-conjugated AffiniPure goat anti-rabbit IgG (Jackson ImmunoResearch Laboratories, Inc., Cat #III-035-003, 1:10000 milk), peroxidase-conjugated AffiniPure goat anti-mouse IgG (Jackson ImmunoResearch Laboratories, inc., Cat #115-035-003, 1:10000 milk). Blots were visualized using ECL Western Blotting Substrate (Pierce, Cat #32106) or SuperSignal West Femto Substrate (Thermo Scientific, Cat #34096) and imaged on a BIO-RAD ChemiDoc Touch Imaging system.

### NF-kB Activation Reporter

THP1-Blue NF-kB SEAP reporter cells were grown in a 96-well plate in triplicate with the indicated inhibitor for 24 hours. In a new 96-well plate, 20 μl of cell supernatant was added to 180 μl of QuantiBlue Reagent (Invivogen, Cat #rep-qbs2) and incubated at 37° C. for 30 minutes. Absorbance was read at 630 nm.

### RNA Sequencing

WT, MyD88^{KO}, IRAK1^{KO}, IRAK4^{KO} and IRAK1/4^{dKO} THP1 cells were plated in triplicate at a uniform density of 5×10⁵ cells per ml of complete media 24 hours prior to RNA collection. For inhibitor studies, cells lines were cultured at a density of 5×10⁵ cell/mL in the presence of IRAK4 inhibitors for 24 hours. Inhibitor studies were performed in duplicate. RNA from IRAK KO and inhibitor-treated cell lines was isolated with the RNeasy Mini Kit (Quiagen). RNA quality was assessed with the Agilent 2100 Bioanalyzer. Sequencing libraries were prepared and sequenced at an average depth of 30M paired-end 100 nucleotide reads. FASTA files were used for alignment to the human genome (GRCh37) with the STAR software to generate BAM files. After the quality of reads was examined using FastQC, paired-end reads were aligned against the UCSC/hg38 genome using HISAT2 (v2.0.5). The raw gene counts were calculated using featureCounts (v1.5.2) and normalized using edgeR. Differentially expressed genes were predicted using limma/voom (v3.30.6).

### Assay for Transposase-Accessible Chromatin (ATAC) Sequencing

ATAC-sequencing was performed using a slightly modified version of the OMNI protocol. Briefly, nuclei were isolated by collecting 50,000 cells per sample and lysing in 10 mM Tris-HCl, pH 7.5/10 mM NaCl/3 mM MgCl₂ supplemented with 0.10% Tween-20, 0.1% NP-40 and 0.01% digitonin. Samples from each condition were collected in triplicate. The Illumina Tagment DNA TDE1 Enzyme kit (Illumina) was used for the transposition and tagmentation of open chromatin regions under the conditions specified by the OMNI protocol. DNA was next purified using the Qiagen MinElute Reaction Clean-up kit, per manufacturer's instructions. For library preparation, tagmented DNA was PCR amplified with the NEBNext High-Fidelity 2X PCR Master Mix using the Nextera i5 common adapter and i7 index adaptors. The amplified library was purified by size-selective precipitation with AMPure XP magnetic beads. Library concentrations were determined with the Qubit fluorometer and the dsDNA HS Assay Kit. Library quality was assessed by running a portion of each sample on an agarose gel with SYBR Safe to visualize nucleosome banding. ATAC libraries were paired-end sequenced at 150 nucleotides per end on an Illumina NovaSeq 6000. After the quality of reads was examined using FastQC, adapters in paired-end reads were trimmed using Trim Galore (v0.6.6). Trimmed reads were aligned against the UCSC/hg38 genome using Bowtie2 (v2.4.2). Duplicated and multi-mapped reads (MAPQ cutoff=30) reads were removed using PICARD (v2.18.22) and Samtools (v1.13.0), respectively. HOMER (v4.11) was used to predict and annotate differential peaks with the following parameters (-size 75-mDist 50-style factor -FDR 0.05). Differential peaks with FDR<0.05 were considered as statistically significant.

### Growth Curves

For growth curves with IRAK4 inhibitors, cells were initially plated at a uniform density in 12-well plates with media containing designated concentrations of drug. Cells were counted every other day, at which point cultures were spun down to completely replace the media with fresh media and drug. Cells were split to maintain appropriate confluency, and dilution factors were recorded to adjust cell counts.

### APEX Affinity Purification

The cDNA sequences for IRAK4 and IRAK1 were optimized using the IDT Codon Optimization tool. To produce N-terminal fusions, gBlocks encoding 5′-APEX2-spacer-V5 tag-IRAK1/4 cDNA-3′ with overhangs for Gibson assembly were obtained from IDT. Due to the large size of IRAK1, the IRAK1 sequence was split into two gBlocks. NEBuilder HiFi DNA Assembly master mix was used to Gibson assemble the IRAK4 and IRAK1 gBlocks into the pCW57.1-eGFP plasmid, which was linearized by digestion with NheI and MluI. pCW57.1-eGFP was generated from Addgene #41393 by substituting the puromycin cassette for eGFP. Assembled APEX2 vectors were transformed into One Shot™ Stbl3™ E. coli and individual colonies were expanded. APEX2 vectors were maxi-prepped and sequenced to confirm the fidelity of the inserts and then transfected into HEK-293T with 3^{rd} generation lentiviral helper plasmids. IRAK4-APEX2, IRAK1-APEX2, and empty vector viral supernatants were collected and used to transduce IRAK4^{KO} and IRAK1^{KO} THP1. Transduced cells were sorted on GFP expression to obtain pure populations. Inducible expression of fusion proteins and functional rescue of canonical signaling was confirmed by immunoblot. To perform the proximity labeling, 2×10⁷ Dox-induced (0.5 mg/ml, 48 hrs) and uninduced cells were plated in 1 ml of pre-warmed media in quadruplicate. Cells were preloaded by incubating with 500 μM biotin phenol for 30 minutes at 37° C. The APEX2 enzyme was then activated by adding H₂O₂ at a final concentration of 1 mM and gently agitating for 45 seconds. The reaction was immediately quenched by adding quenching buffer (PBS with 10 mM sodium azide, 10 mM sodium ascorbate, 5 mM Trolox). Cells were washed with quenching buffer two additional times and then lysed and sonicated in 500 uL RIPA containing protease inhibitor and quenchers. Lysates were rotated overnight with Sera-Mag blocked streptavidin SpeedBeads to precipitate biotinylated proteins. Beads were washed and stored at −80° C. prior to protein identification by mass spectrometry. Beads from one replicate each of the uninduced controls and dox-induced samples were eluted with sample buffer containing 20 mM DTT. Eluents were run on an SDS gel which was stained with imperial stain to ensure capture of biotinylated proteins above background in the dox-induced samples.

### Statistical Analysis

The number of animals, cells, and experimental/biological replicates can be found in the figure legends. Differences among multiple groups were assessed by one-way analysis of variance (ANOVA) followed by Tukey's multiple comparison posttest for all possible combinations. Comparison of two group was performed using the Mann-Whitney test or the Student's t test (unpaired, two tailed) when sample size allowed. Unless otherwise specified, results are depicted as the mean±standard deviation or standard error of the mean. A normal distribution of data was assessed for data sets >30. For correlation analysis, Pearson correlation coefficient (r) was calculated. For Kaplan-Meier analysis, Mantel-Cox test was used. All graphs and analysis were generated using GraphPad Prism software or using the package ggplot2 from R.

### Results

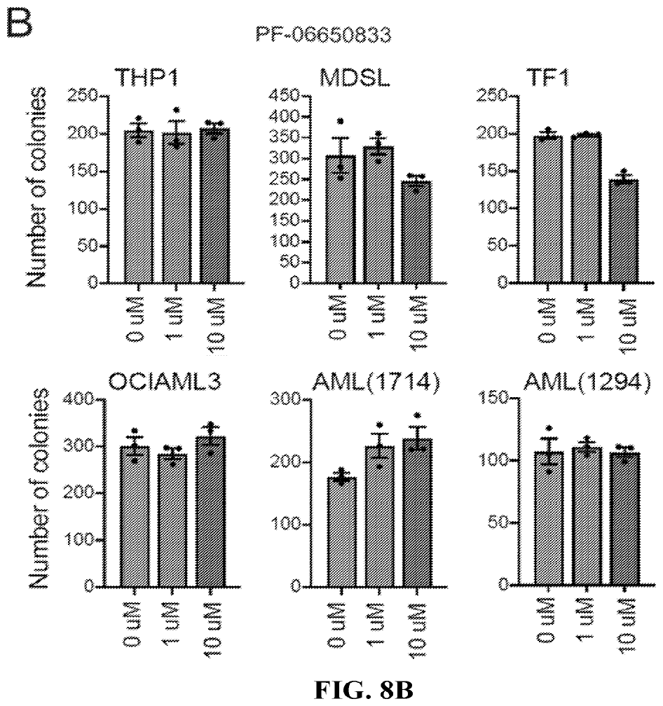

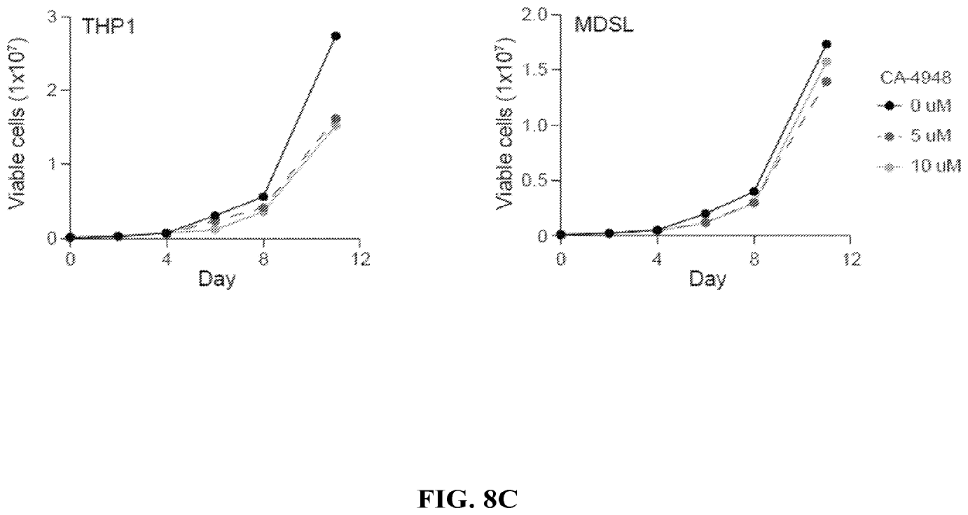

IRAK4 inhibition or deletion results in compensatory activation of IRAK1 in MDS/AML To interrogate the role of IRAK4 in HR-MDS/AML, a panel of cell lines and patient-derived samples was treated with two clinical-stage IRAK4 inhibitors, PF-06650833 (“PF-066”) and CA-4948 (Table 14), and cell viability and LSPC function was evaluated in methylcellulose. As expected, both IRAK4 inhibitors suppressed TLR-mediated NF-kB activation at sub-micromolar concentrations (PF-06650833, IC₅₀=7 nM; CA-4948, IC₅₀=500 nM) (FIG. 8A). Next, the effect of IRAK4 inhibitors on in vitro hematopoietic progenitor cell-mediated colony formation was evaluated in methylcellulose, a surrogate assay to assess LSPC properties.

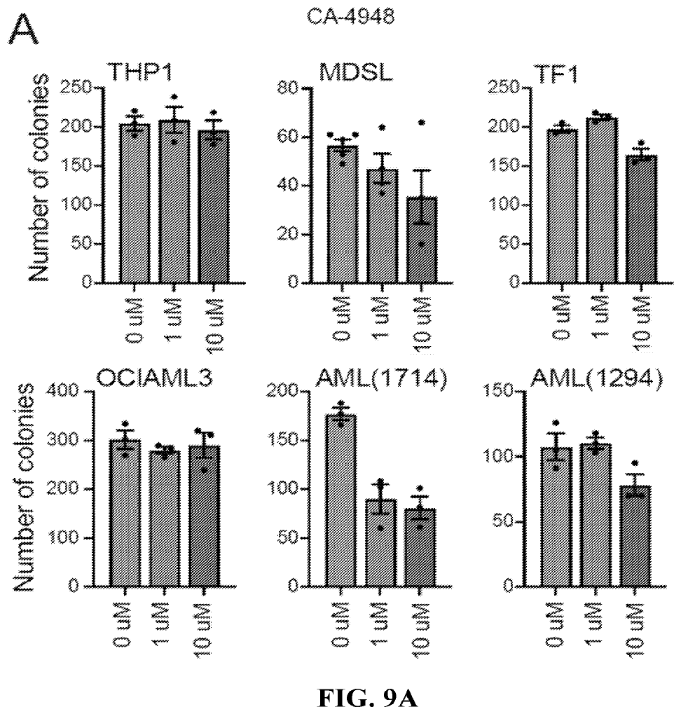

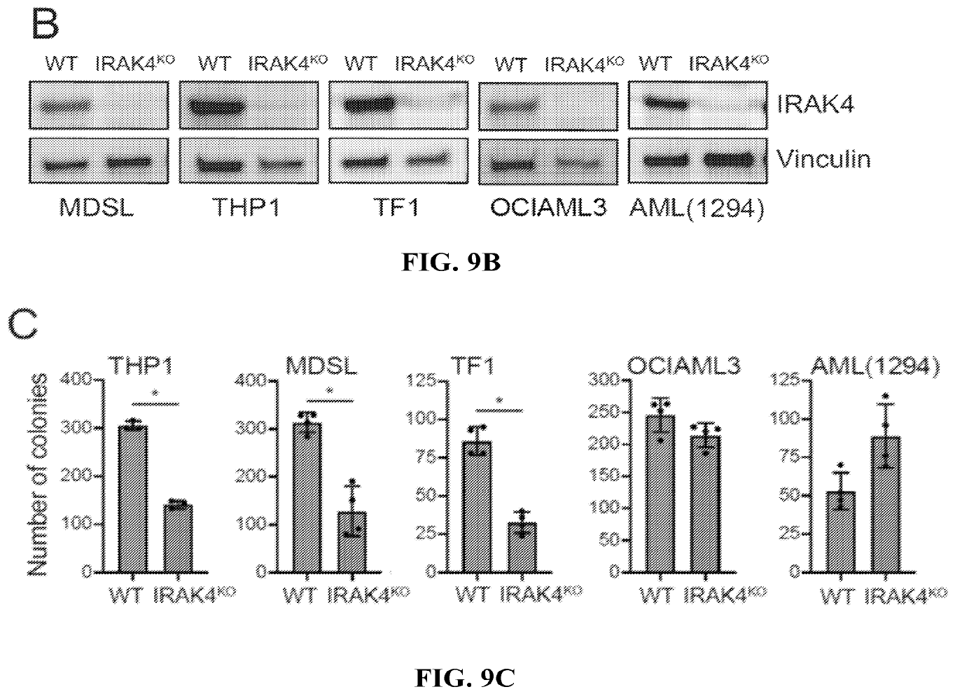

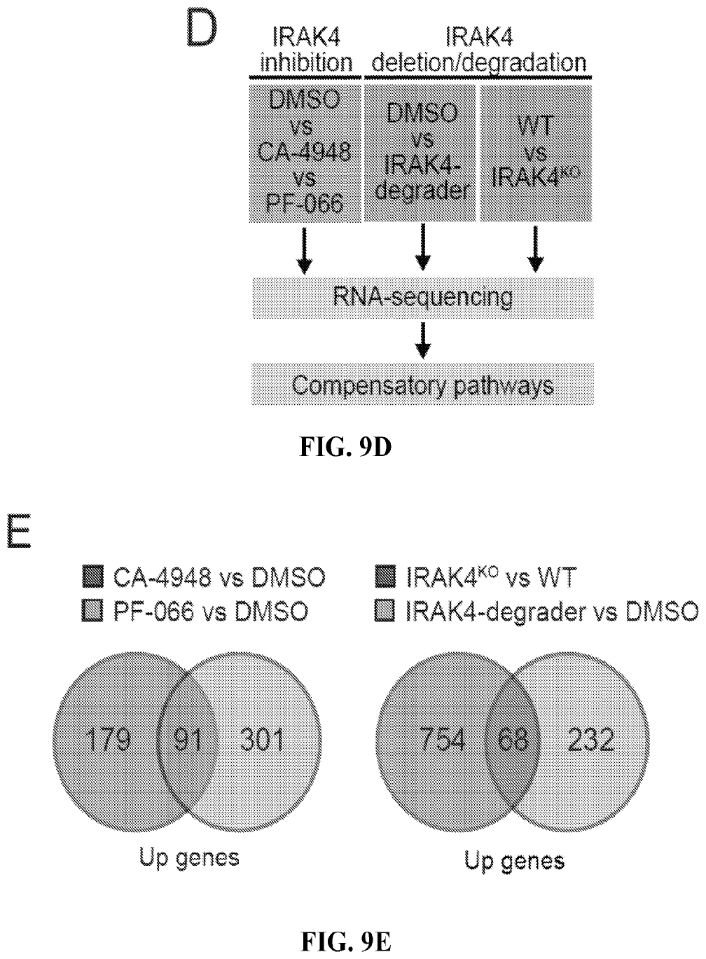

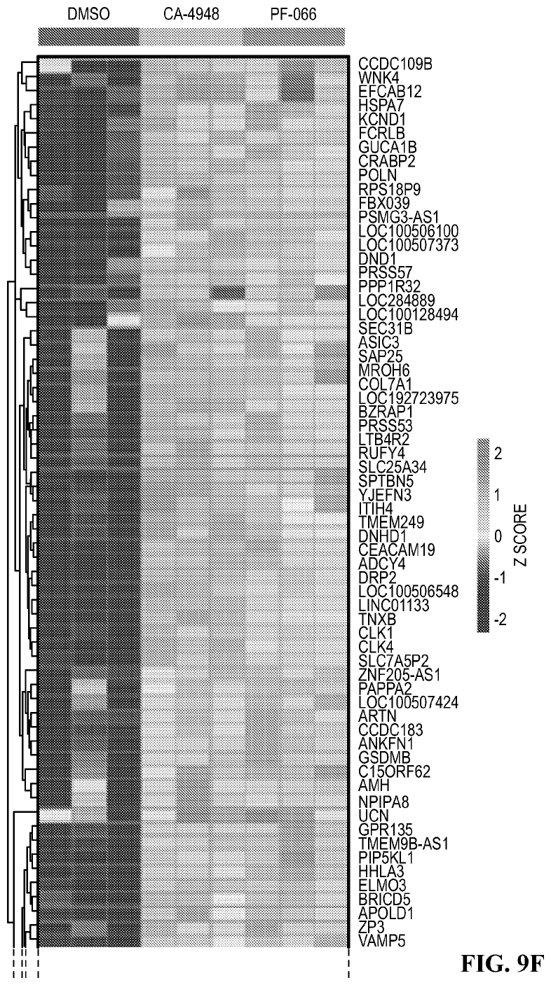

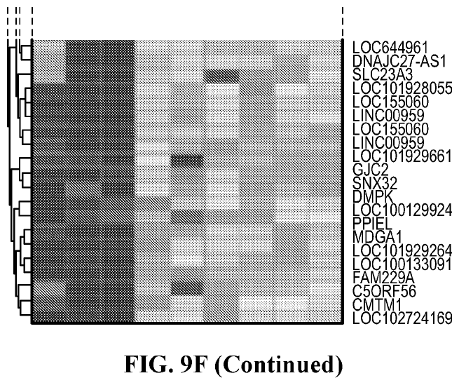

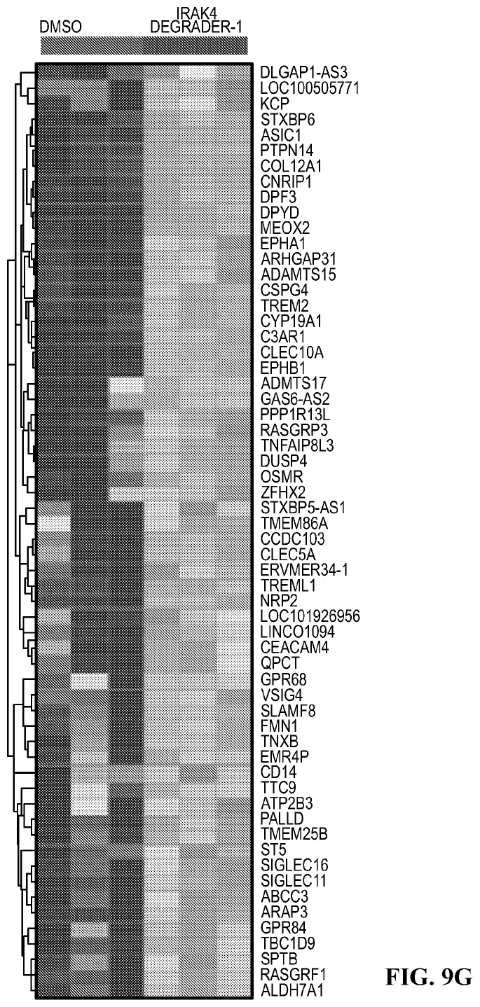

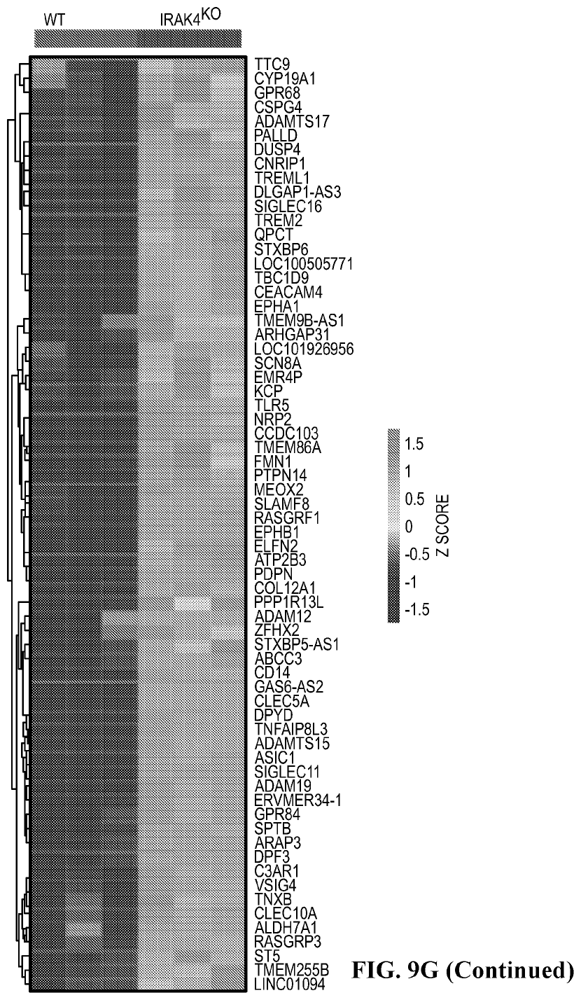

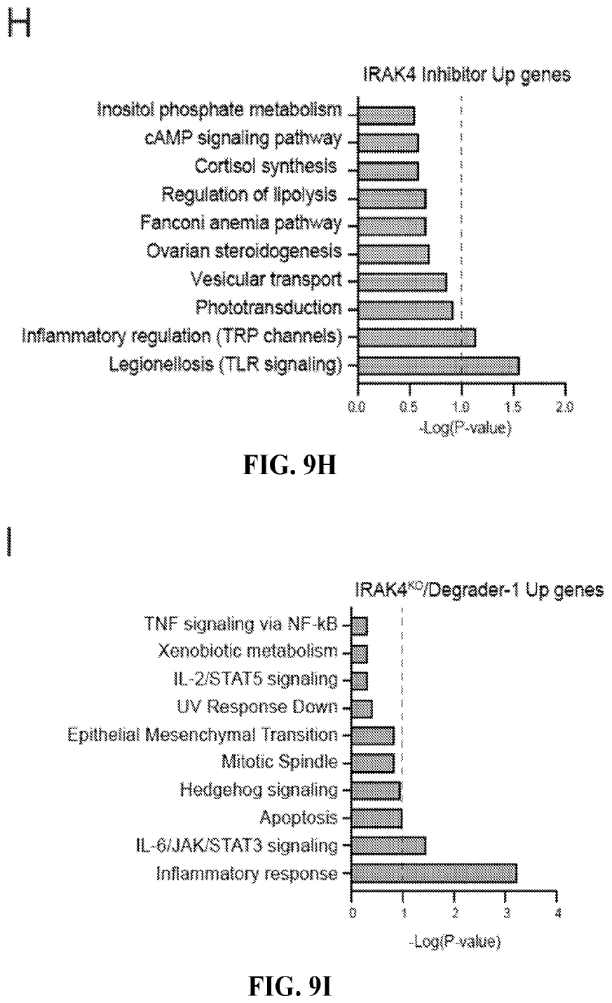

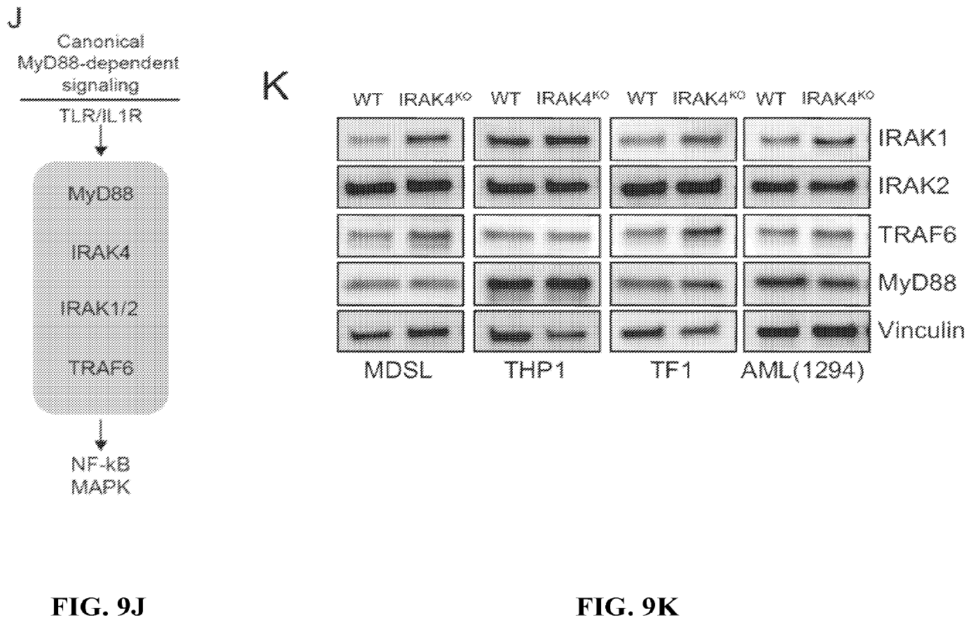

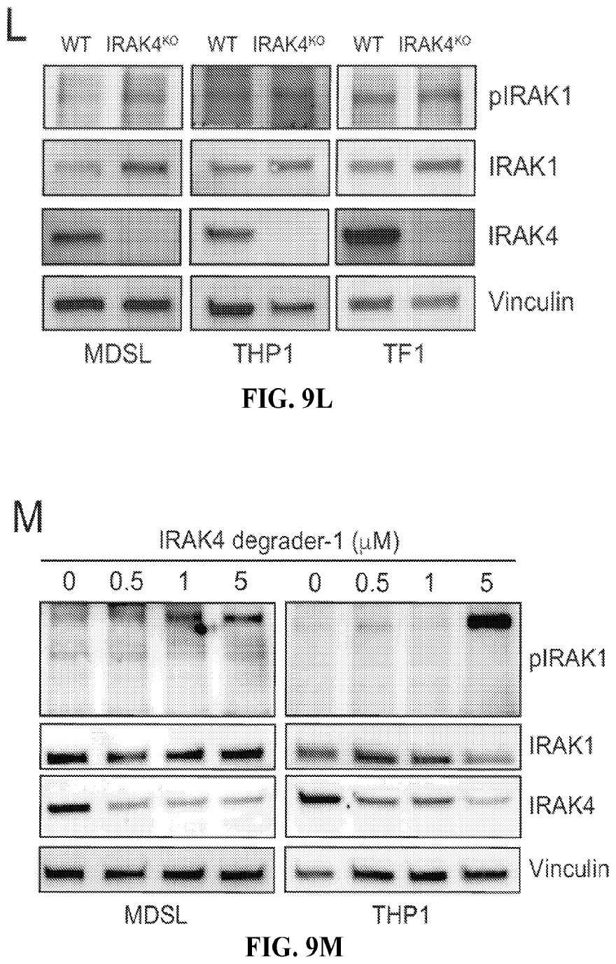

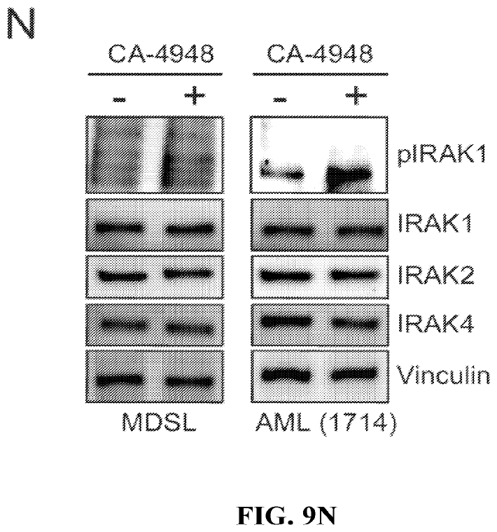

Consistent with previous reports, emavusertib (CA-4948) treatment resulted in a dose-dependent, yet incomplete, inhibition of colony formation in a subset of MDS/AML cell lines (MDSL and TF1) and patient-derived AML samples (AML1714 and AML1294) (FIGS. 9A-9N). PF-06650833 also resulted in inhibition of colony formation by MDSL and TF1 cells but was less effective at suppressing the patient-derived AML samples (FIG. 8B). The proliferation and cell survival of MDS (MDSL) and AML (THP1) cells was only modestly suppressed following treatment with CA-4948 (FIG. 8C), suggesting that IRAK4 inhibition is not cytotoxic but rather suppresses LSPC function. To confirm that the effects observed with the IRAK4 inhibitors were mediated by targeting IRAK4, a panel of IRAK4-deficient isogenic cell lines (IRAK4^{KO}) was generated using CRISPR/Cas9 (FIG. 9B). Generally consistent with the effects observed with the IRAK4 inhibitors, deletion of IRAK4 resulted in a significant, although moderate, reduction in leukemic progenitor cell colonies in a subset of MDS/AML cell lines (FIG. 9C). For OCIAML3 and AML(1294), pharmacologic or genetic targeting of IRAK4 did not result in suppression of colony formation, suggesting that inhibition of IRAK4 alone in these samples is not sufficient to suppress LSPC function. As observed with the IRAK4 inhibitor studies above, deletion of IRAK4 did not significantly affect MDS or AML cell survival (data not shown). Collectively, IRAK4 inhibition incompletely suppresses MDS/AML LSPC function in a subset of evaluated samples, recapitulating the observations from the single-agent clinical trials for HR-MDS and AML.

TABLE 14
Cell lines and patient-derived samples

| | | | Relevant mutations/ |
|---|---|---|---|
| Specimen | Diagnosis | IRAK4-L* | cytogenetics |
| AML(1714) (2017-114) | AML | Yes | MLL-AF10, PTPN11, |
| | | | del(11)(q21q23) |
| AML(1294)(2017-94) | AML | Yes | CDKN2A, CDKN2B |
| AML(64519) | AML | ND | TET2, EZH2, ASXL1, |
| | | | CEBPA, JAK2 |
| AML(08) | AML | ND | Del(5), −3, del(17), +8, −15, |
| AML(0169)(2016-9) | AML | ND | EGFR, FLT3, FLT3-ITD, MLL- |
| | | | MLLT4, HOOK3-MYST3 |
| MDS(76960) | MDS-RCMD | ND | ETV6 |
| MDS(3328) | MDS | ND | U2AF1 |
| THP1 | AML (M5) | Yes | MLL-AF9, ATM, FANCD2, |
| | | | NRAS, TP73 |
| MDSL | HR-MDS | Yes | CEBPA, NRAS, TET2, |
| | | | TP53, del(5q) |
| TF1 | AML (M6) | Yes | MAP3K1, NRAS, SF3B1, TP53 |
| OCIAML3 | AML (M4) | Yes | NPM1, NRAS |

To explore potential mechanisms for the moderate and incomplete responses to IRAK4 inhibitors in MDS/AML, gene expression changes in AML cells upon deletion or inhibition of IRAK4 were examined. RNA sequencing was performed on THP1 cells treated with the IRAK4 inhibitors (CA-4948, PF-06650833) or a PROTAC (IRAK4 degrader-1), and isogenic wild-type (WT) and IRAK4^{KO} THP1 cells. To identify potential compensatory pathways activated upon either IRAK4 kinase inhibition or IRAK4 degradation, the analysis was initially focused on upregulated genes (Log₂ FC>1.5, q value >0.05) (FIG. 9D and Tables 2-5 in Appendix A). There were 91 common upregulated genes following treatment with CA-4948 and PF-06650833 relative to vehicle-treated cells (FIGS. 9E and 9F). In THP1 cells following genetic deletion (IRAK4^{KO}) or pharmacologic degradation with a PROTAC (IRAK4 degrader-1) of IRAK4, 68 common upregulated genes were identified relative to the respective control cells (FIG. 9E and FIG. 9G). Unexpectedly, pathway analysis of the overlapping upregulated genes corresponded with significant enrichment for “TLR signaling” (P=0.027) upon IRAK4 kinase inhibition (FIG. 9H) and “Inflammatory response” (P=0.0006) and IL-6/JAK/STAT3 signaling” (P=0.035) upon IRAK4 deletion (FIG. 9I). Since inflammatory-related signaling pathways were paradoxically activated in IRAK4-inhibited and -deleted AML cells, it was posited that compensatory signaling via the MyD88 complex may contribute to the limited efficacy of TRAK4 inhibitors in leukemic cells. The proximal upstream (MyD88) and downstream (IRAK1, IRAK2, and TRAF6) effectors of canonical IRAK4 signaling were then focused on (FIG. 9J). IRAK1, IRAK2 and IRAK4 are also closely related paralogs with shared structural and functional elements. Immunoblotting of MDS/AML cell lines and patient-derived AML samples revealed increased protein expression of IRAK1 and TRAF6, but not MyD88 nor IRAK2, in IRAK4^{KO} MDS/AML cells as compared to control cells (FIG. 9K). Importantly, RAK1 phosphorylation, which is an indication of its activation state, was also increased upon deletion of IRAK4 in the MDS/AML cells (FIG. 9L). To determine whether IRAK4 kinase inhibition similarly results in IRAK1 activation, immunoblotting of MDS/AML cell lines and patient-derived AML samples treated with the IRAK4 inhibitors or the IRAK4 PROTAC (IRAK4 degrader-1) was performed. Treatment with IRAK4 degrader-1 resulted in a dose-dependent decrease in IRAK4 protein expression and a reciprocal increase in IRAK1 phosphorylation (FIG. 9M). Similarly, CA-4948 resulted in increased IRAK1 phosphorylation in MDSL cells and in a patient-derived AML sample (FIG. 9N). Although the IRAK4 PROTAC and kinase inhibitors both induced IRAK1 activation, they did not result in increased total IRAK1 proteins. This discrepancy with the IRAK4^{KO} MDS/AML cells is likely due to the differences in duration of the experimental conditions. Nevertheless, IRAK4 deficiency or kinase inhibition corresponds with IRAK1 activation in MDS/AML cells.

### Co-Targeting IRAK1 and IRAK4 is Required for Maximal Suppression of LSPCs

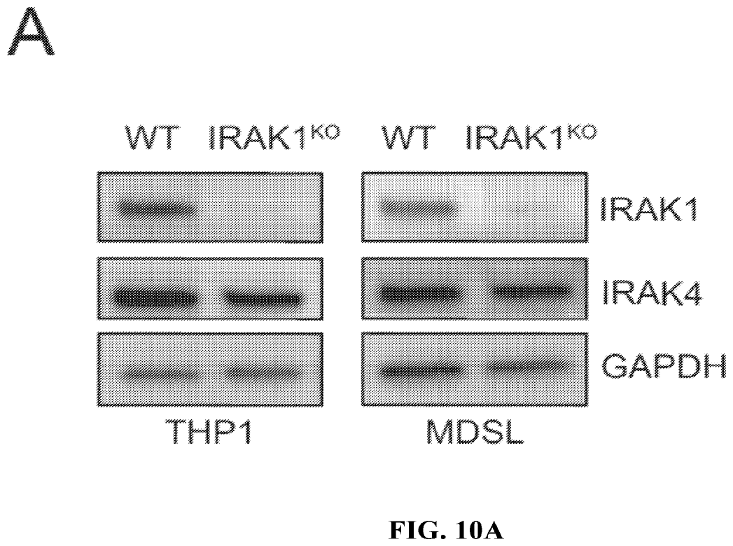

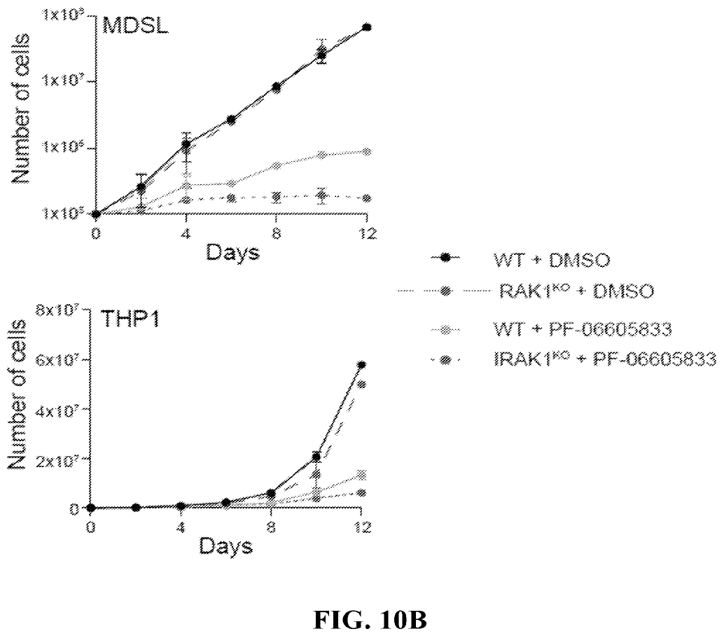

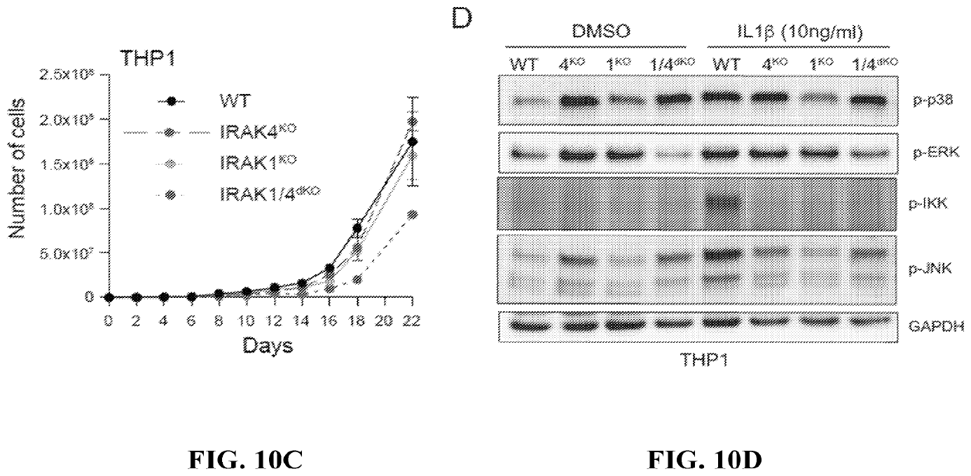

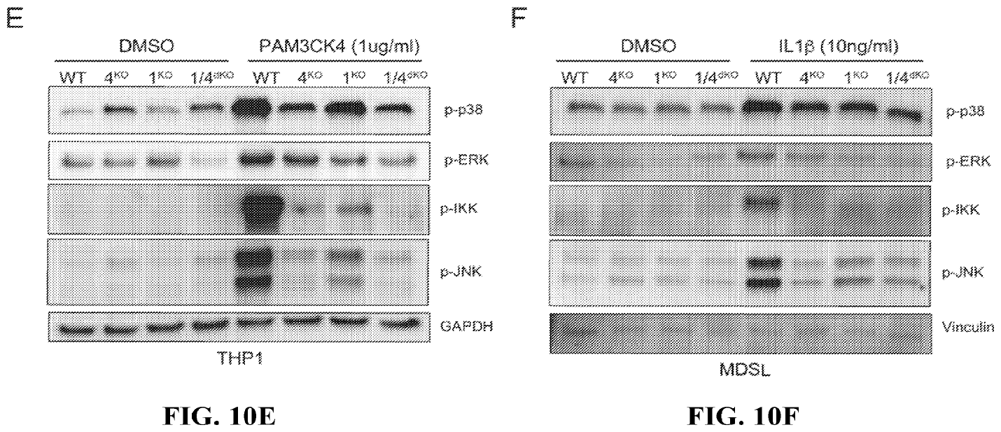

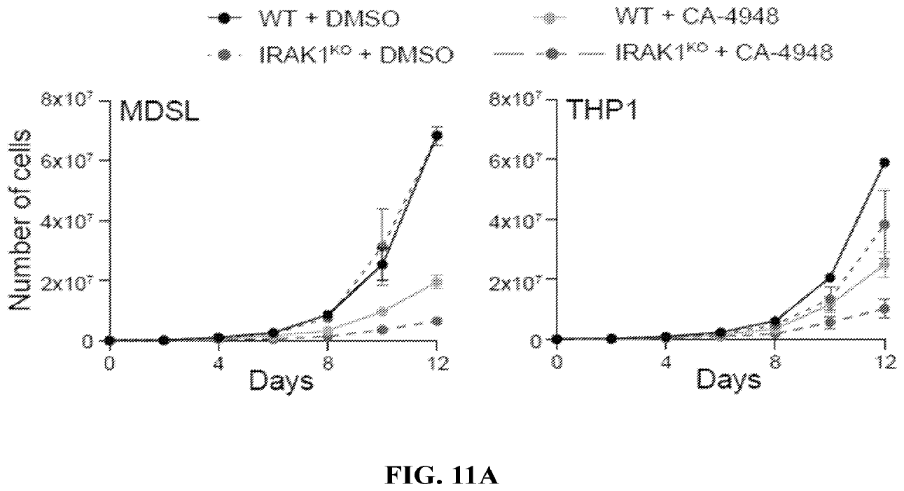

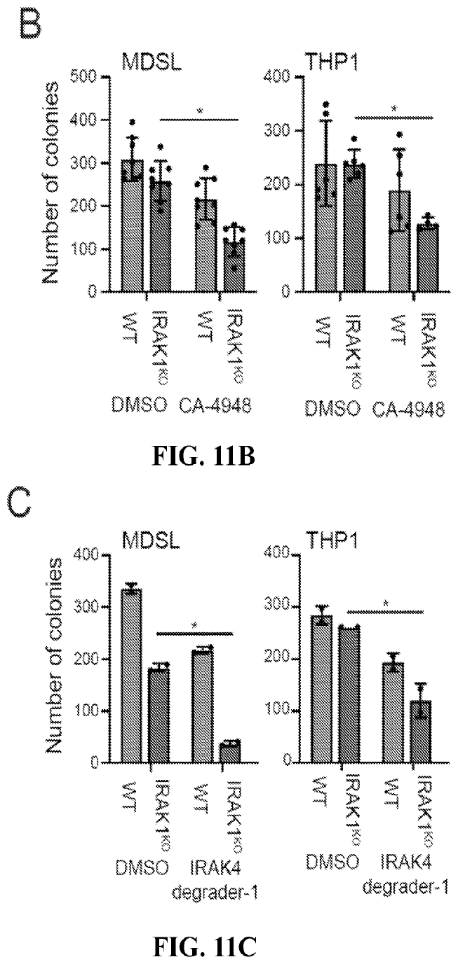

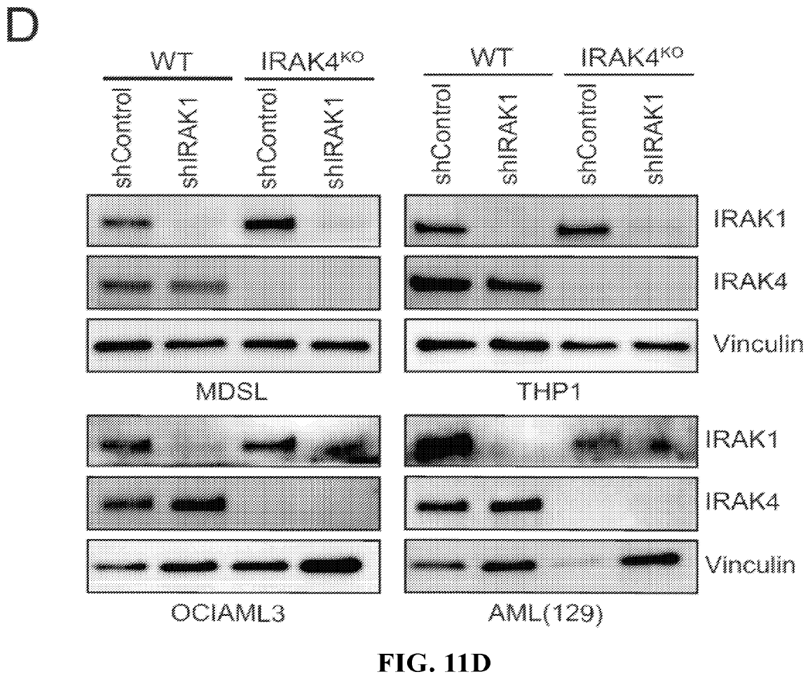

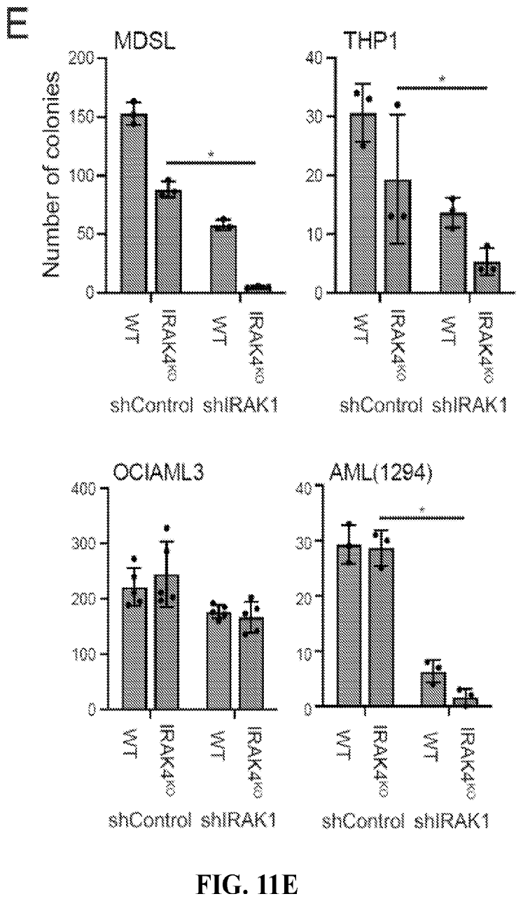

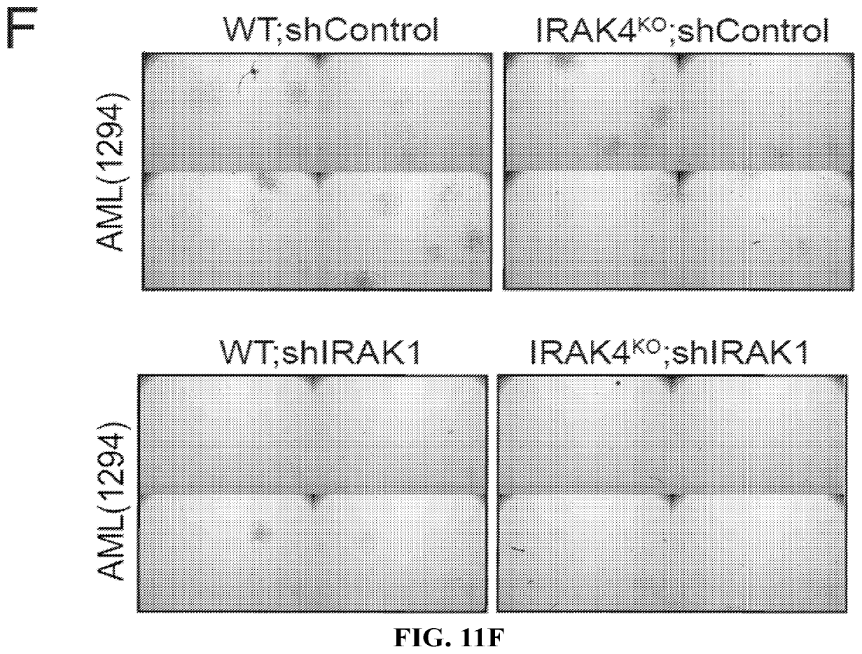

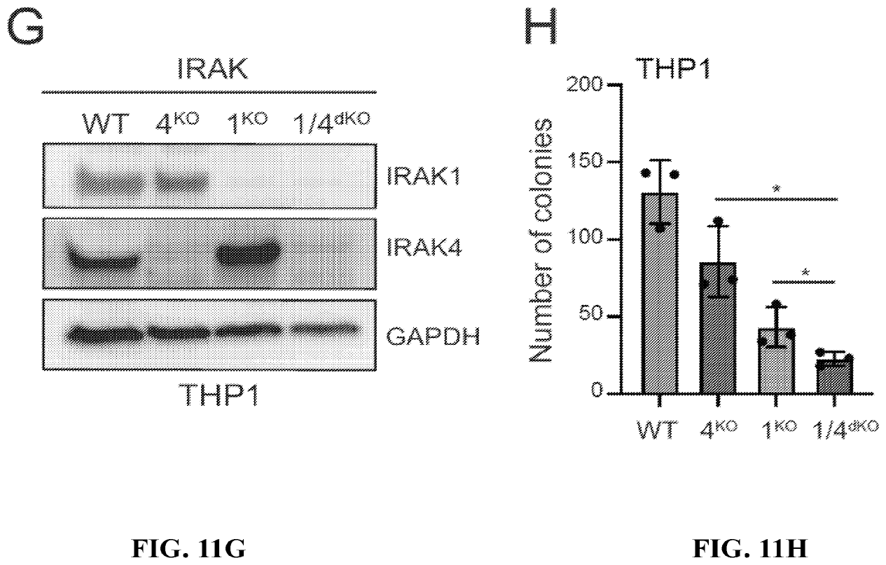

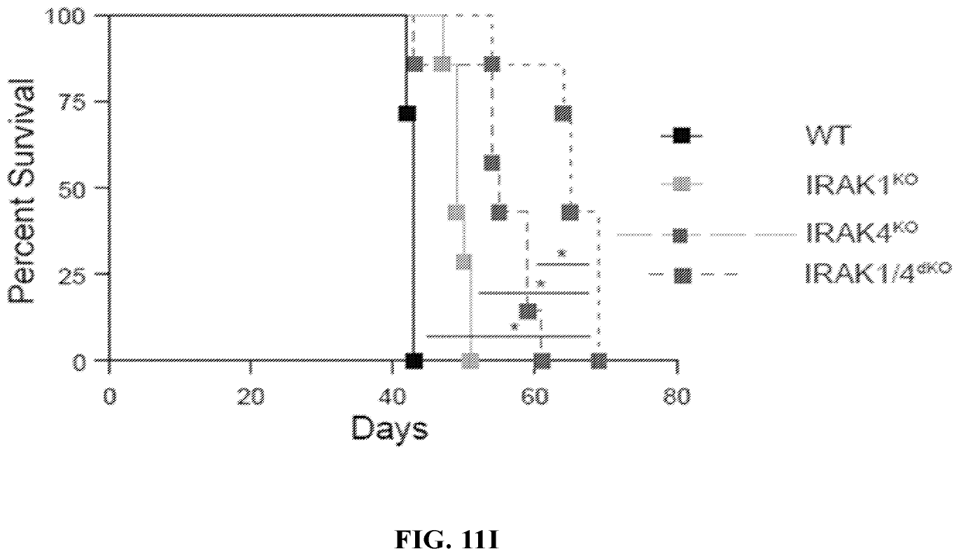

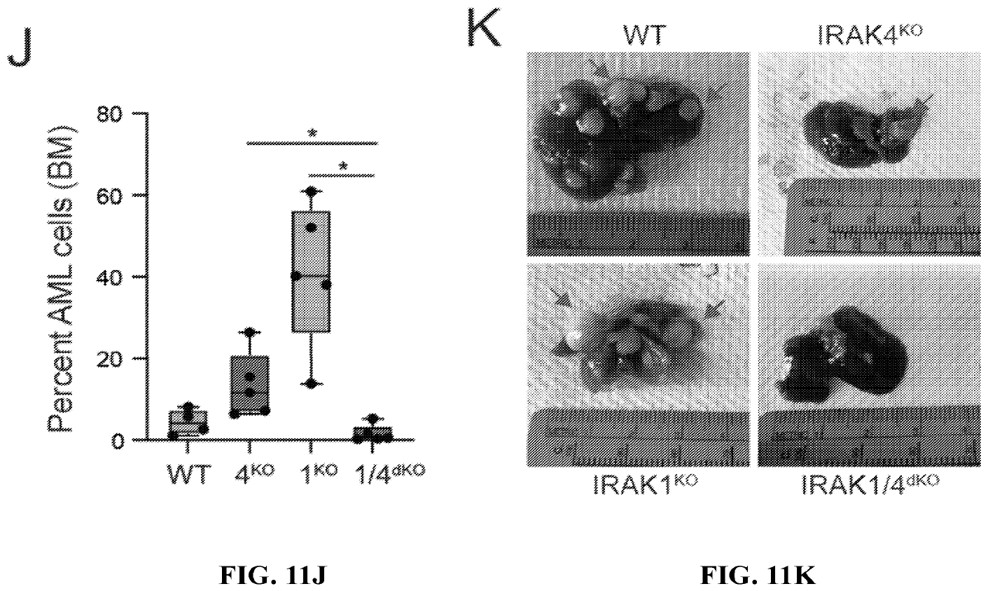

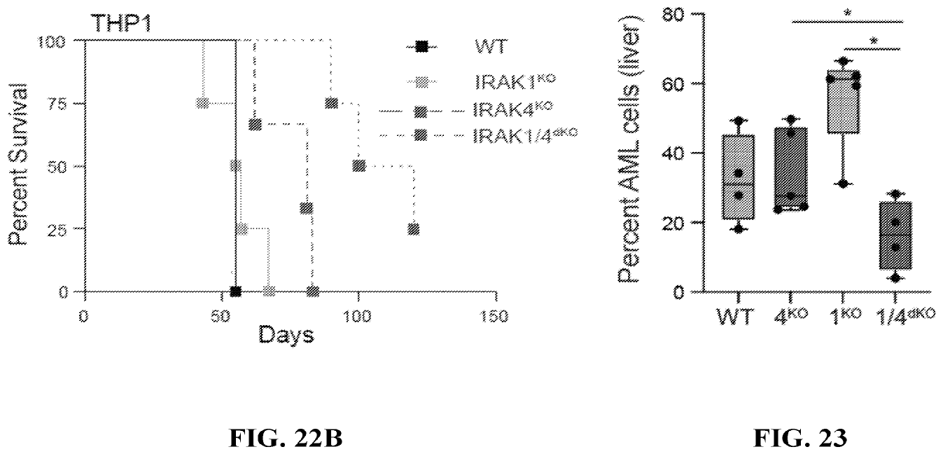

Since IRAK1 is activated upon inhibition or deletion of IRAK4, it was speculated that targeting IRAK1 would complement IRAK4 inhibitor treatment in MDS/AML. Using CRISPR/Cas9, IRAK1-deficient (IRAK1^{KO}) THP1 and MDSL cell lines were generated (FIG. 10A). IRAK1^{KO} MDS/AML cells exhibited similar growth kinetics and leukemic progenitor cell colony formation as WT THP1 and MDSL (FIGS. 11A-11B). However, IRAK1^{KO} MDS/AML cells were more sensitive to treatment with IRAK4 inhibitors (CA-4948) or the PROTAC (IRAK4 degrader-1) (FIGS. 11B-11C). A similar effect was also observed on IRAK1^{KO} MDS/AML cells treated with PF-06650833 (FIG. 10B). To confirm these observations with orthogonal approaches, IRAK1 was first knocked down using shRNAs expressed from lentiviral vectors in the isogenic WT and IRAK4^{KO} MDS/AML cell lines and patient-derived AML samples (FIG. 11D). While OCIAML3 remained less sensitive to deletion of IRAK1 and IRAK4, combined deficiency of IRAK1 and IRAK4 in THP1, MDSL, and AML(1294) resulted in a significant reduction in leukemic progenitor cell colonies as compared to deficiency of either IRAK4 or IRAK1 alone (FIGS. 11E-11F). CRISPR/Cas9 was next used to generate isogenic double IRAK1 and IRAK4 deficient (IRAK1/4^{dKO}) THP1 cells (FIG. 11G). IRAK1/4^{dKO} THP1 cells expanded at a moderately slower rate in liquid culture as compared to IRAK1^{KO} or IRAK4^{KO} cells (FIG. 10C). However, IRAK1/4^{dKO} THP1 cells formed significantly fewer leukemic progenitor cell colonies as compared to IRAK1^{KO} or IRAK4^{KO} cells in the initial plating (FIG. 11H) as well as a secondary replating assay (FIG. 22A). To assess the consequences of combined IRAK1/4 deficiency on leukemia development in vivo, WT, IRAK4^{KO} IRAK1^{KO} and IRAK1/4^{dKO} THP1 cells were xenografted into NOD-scid IL2Rgnull-3/GM/SF (NSGS) mice. Mice engrafted with IRAK1/4^{dKO} THP1 cells survived longer than mice engrafted with either WT (P=0.004), IRAK1^{KO} (P=0.0002), or IRAK4^{KO} cells (P=0.0022) (FIG. 11I and FIG. 22B). Histology samples collected at time of death revealed significantly reduced leukemia cell infiltration of IRAK1/4^{dKO} THP1 cells in the bone marrow (BM) (huCD33+CD15+) and liver (presence of nodules) as compared mice engrafted with WT, IRAK1^{KO}, or IRAK4^{KO} cells (FIGS. 11J-11K and FIG. 23). Thus, concomitant targeting of IRAK1 elicits an exaggerated LSPC defect in IRAK4-deficient MDS/AML models.

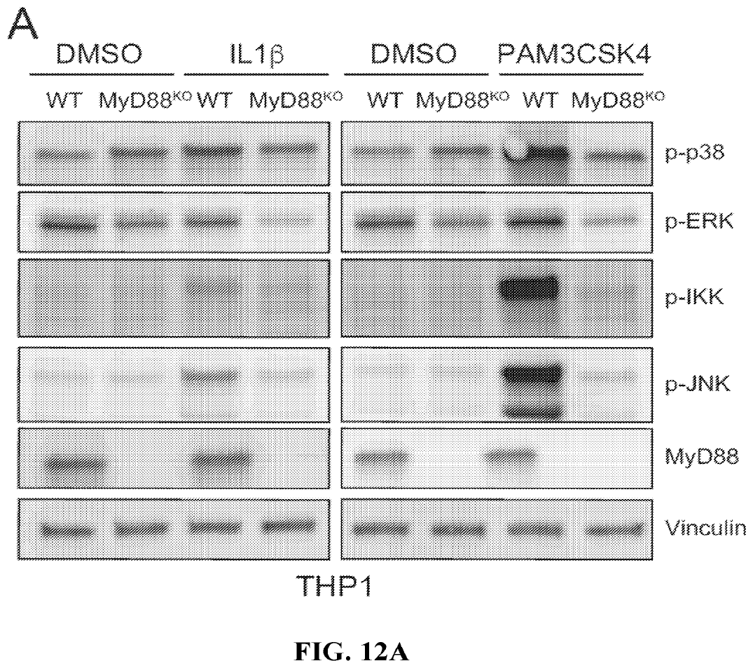

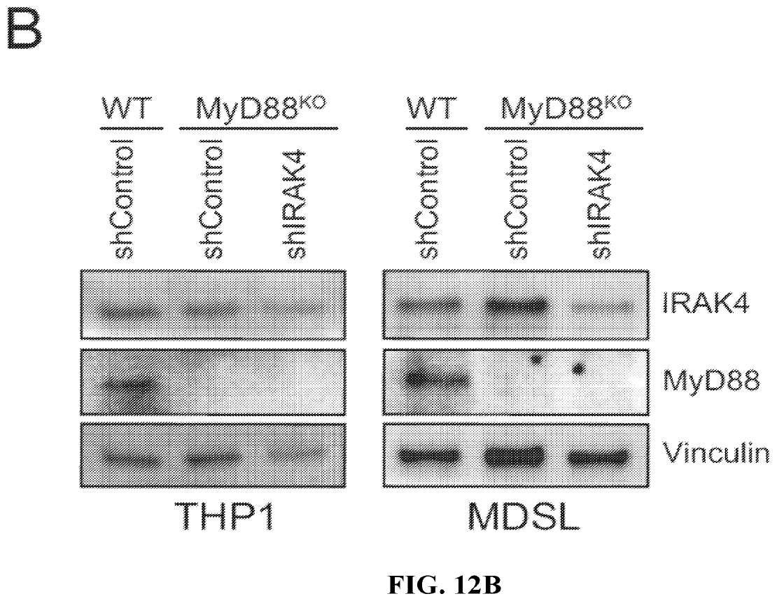

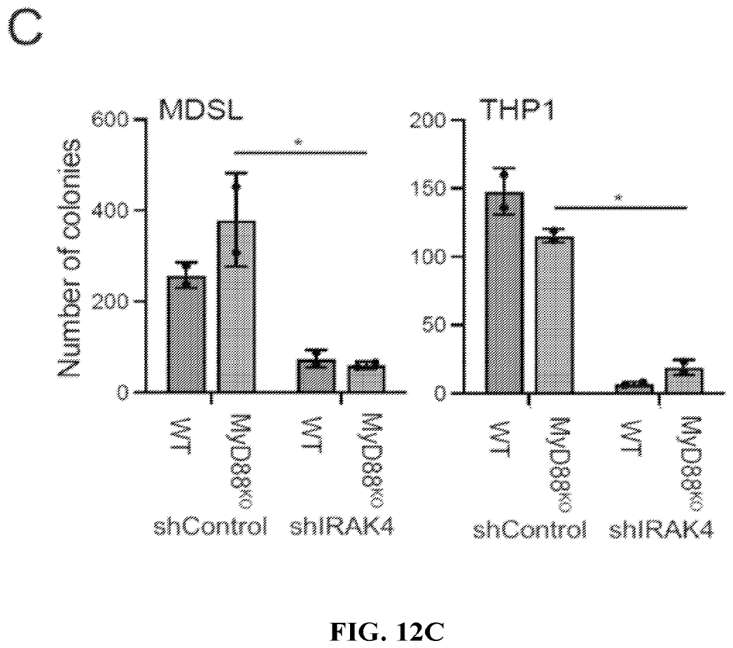

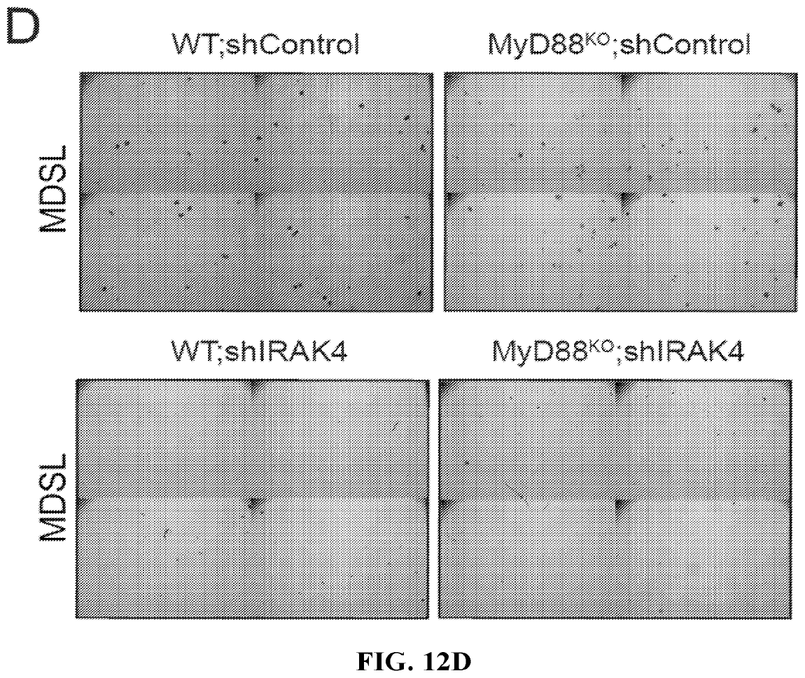

IRAK1 and IRAK4 dependency in MDS % AML, is independent of canonical MyD88 signaling IRAK1 and IRAK4 are presumed to transduce canonical MyD88-dependent signaling in a mutually dependent manner, wherein IRAK4 requires IRAK1 as a downstream effector upon MyD88 activation (FIG. 9J). Thus, singular loss of either kinase should be sufficient to abrogate signaling. However, this paradigm is inconsistent with the observed exaggerated defect of leukemic cells upon concomitant targeting of IRAK1 and IRAK4. Based on these observations, it was hypothesized that IRAK1 and IRAK4 exhibit a degree of functional redundancy downstream of MyD88 signaling in leukemic cells. To test this possibility, the consequences of IRAK1 and IRAK4 deletion on IL1R and TLR2 activation in THP1 cells was examined. As previously reported, deletion of either IRAK4 or IRAK1 was sufficient to prevent activation of NF-kB (pIKKb) upon stimulation with IL-1b (via IL-1R) (FIG. 10D). As a notable exception, phosphorylation of IKKb upon TLR2 stimulation with PAM3CSK4 was partially reduced in IRAK4^{KO} and IRAK1^{KO} THP1 cells, but completely ablated in the IRAK1/4^{dKO} cells (FIG. 10D). IRAK4 deletion was also sufficient to block phosphorylation of JNK and p38 upon TLR2 stimulation with PAM3CSK4 (FIG. 10E). In addition, it was observed that activation of MAPK was increased at baseline in IRAK4^{KO} THP1 cells, which is consistent with the observed enrichment of inflammatory signatures in the gene expression analysis. Similar signaling dependencies on IRAK1 and IRAK4 were observed in MDSL cells (FIG. 10F). The role of MyD88 in MDS and AML cells was next explored. Deletion of MyD88 in THP1 and MDSL cells using CRISPR/Cas9 confirmed that downstream NF-kB and MAPK pathway activation was terminated in MyD88^{KO} THP1 and MDSL cells upon IL1R or TLR2 stimulation (FIG. 12A), reaffirming an absolute requirement for MyD88 during canonical signaling.

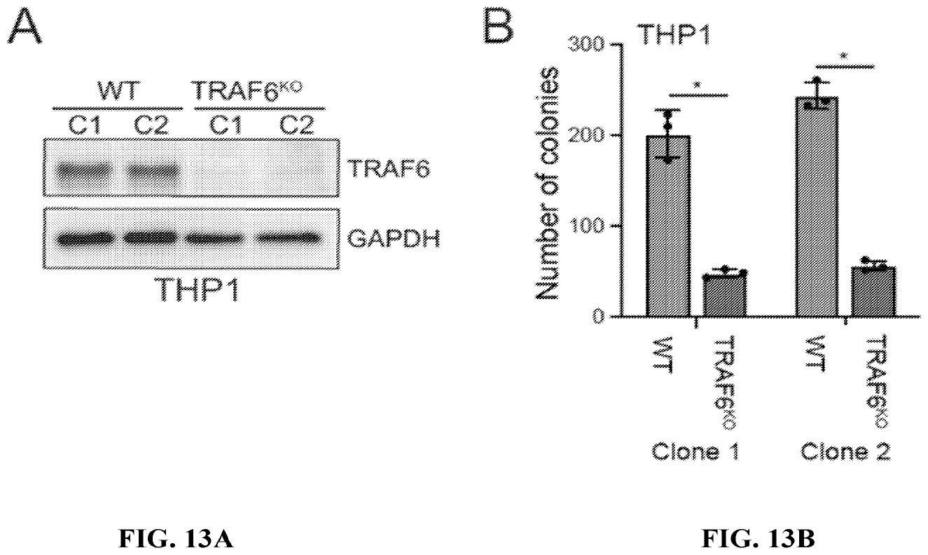

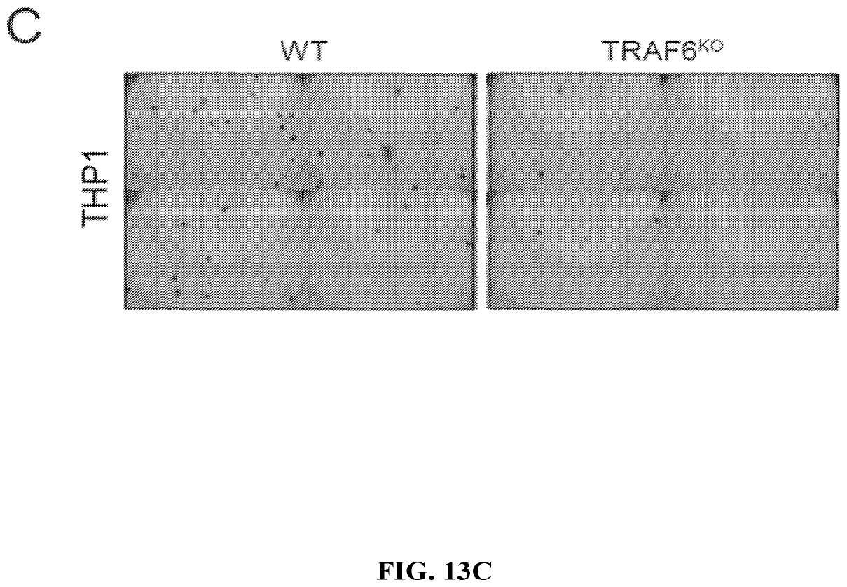

If compromised canonical MyD88-dependent signaling underlies the IRAK1/4^{dKO} phenotype, deletion of MyD88 should phenocopy IRAK1/4^{dKO} MDS/AML cells. Surprisingly, MyD88^{KO} THP1 or MDSL cells formed equivalent number of colonies as compared to WT cells, indicating that MyD88 is dispensable for leukemic progenitor cell function (FIGS. 12B-12C). The dependency of MyD88^{KO} AML cells to loss of IRAK1/4 was next assessed. Knockdown of IRAK4 using shRNAs expressed from lentiviral vectors in isogenic WT and MyD88^{KO} THP1 and MDSL cell lines resulted in a significant reduction of leukemic progenitor cell function (FIGS. 12B-12D). Thus, IRAK1 and IRAK4 exhibit functions that are uncoupled from canonical MyD88 signaling in MDS/AML cells. Since it was observed that TRAF6, the proximal downstream effector of IRAK1/4 signaling (FIG. 9J), is upregulated upon deletion of IRAK4 in MDS/AML cells (FIG. 9K), it was next discerned whether TRAF6 remains relevant to MyD88-independent IRAK1/4 signaling. Deletion of TRAF6 in THP1 and MDSL cells using CRISPR/Cas9 resulted in a significant reduction in leukemic progenitor cell colonies (FIGS. 13A-13C), suggesting that MyD88-independent IRAK1/4 signaling requires TRAF6 as an effector. Collectively, these findings demonstrate that IRAK1 and IRAK4 mediate MyD88-independent functions that are critical for MDS and AML LSPC function.

### Non-Canonical IRAK1/4 Signaling is Essential for Maintaining LSPC States in MDS/AML

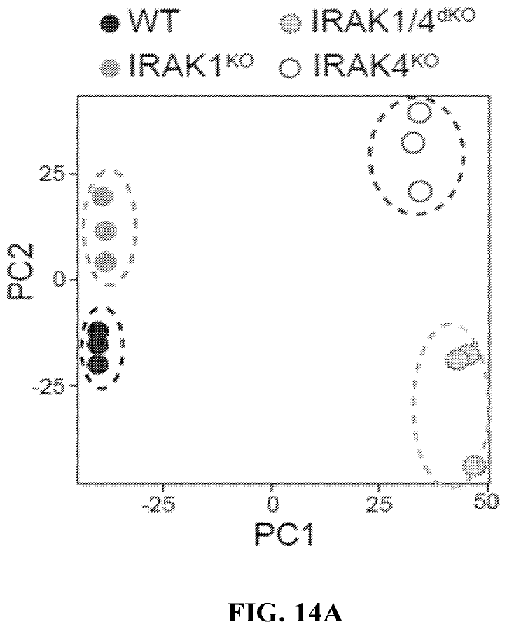

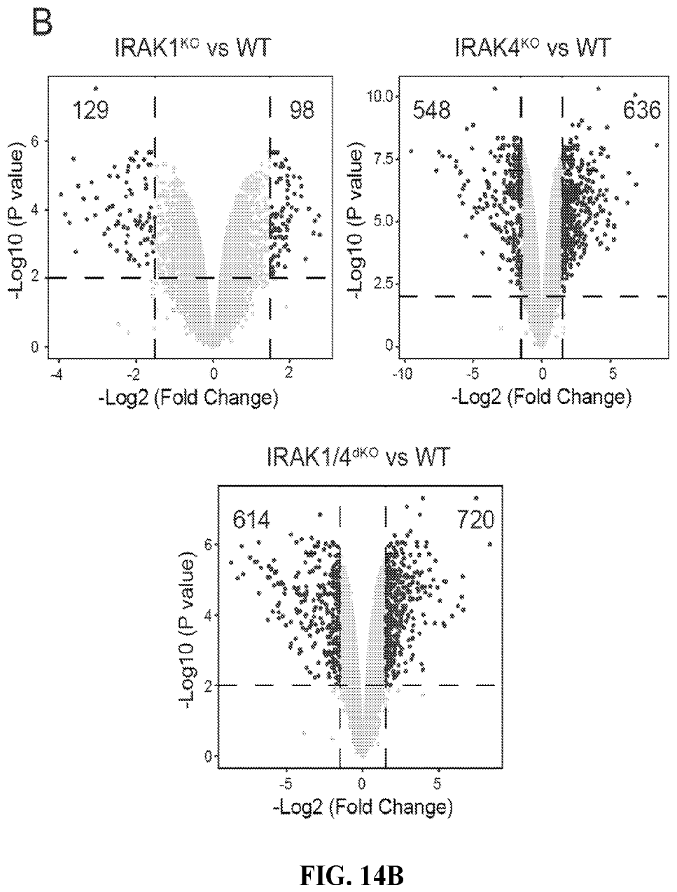

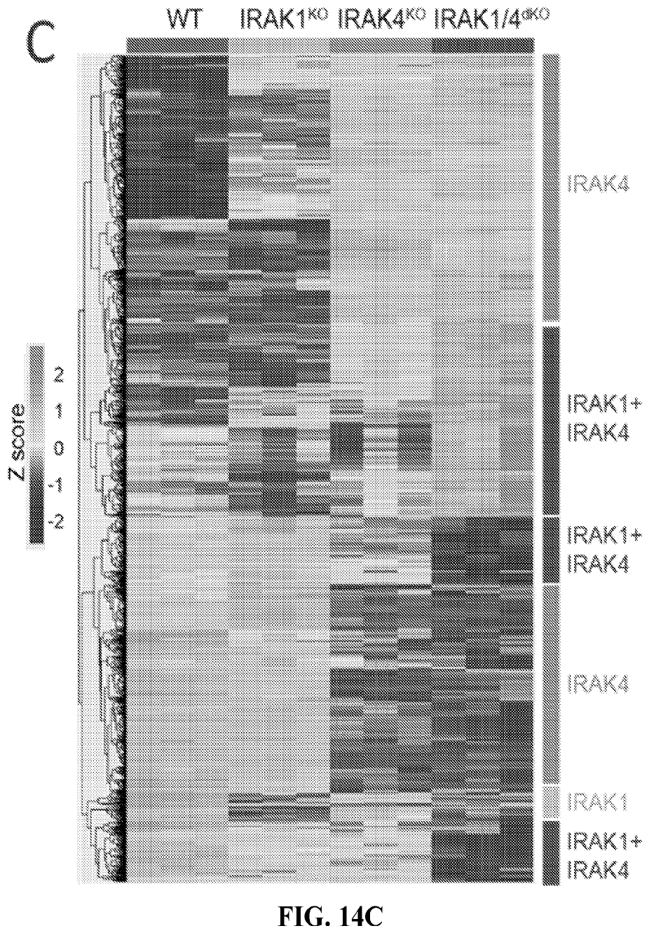

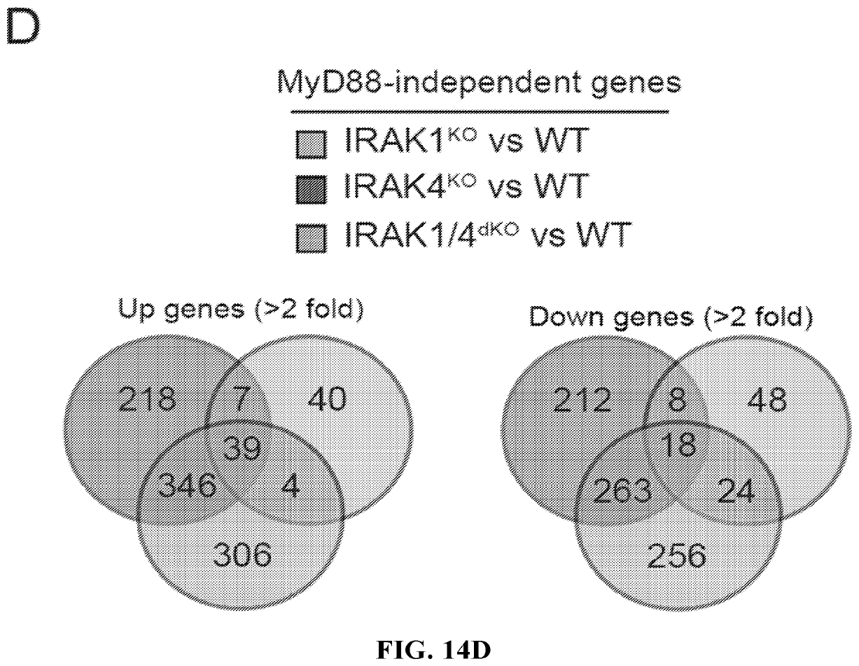

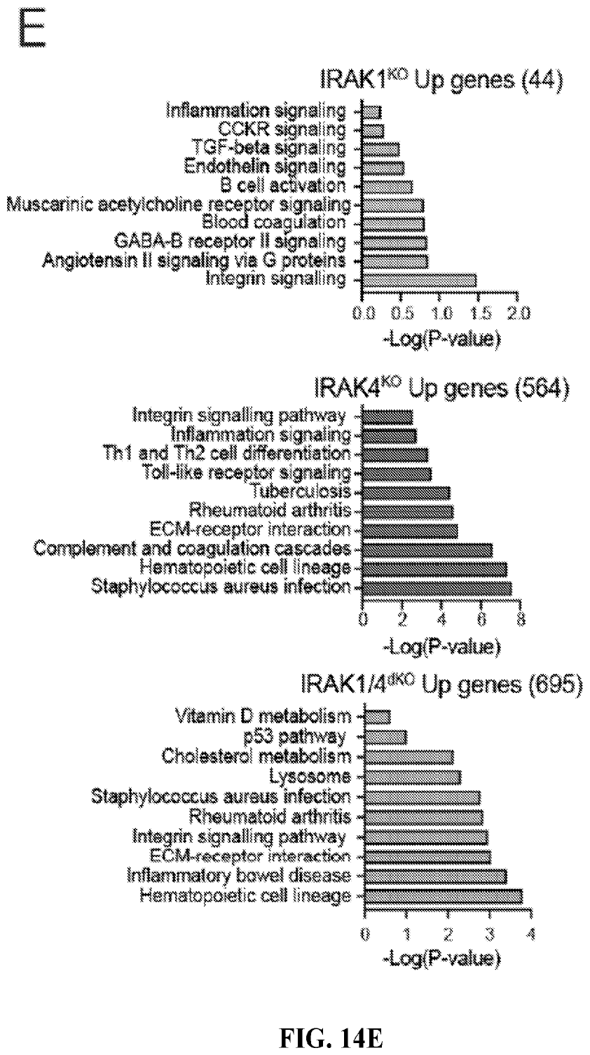

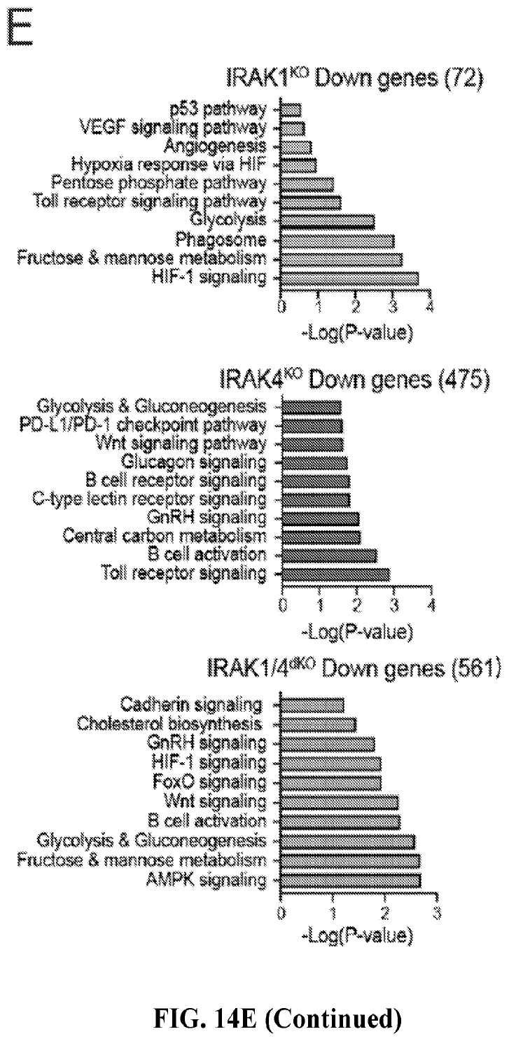

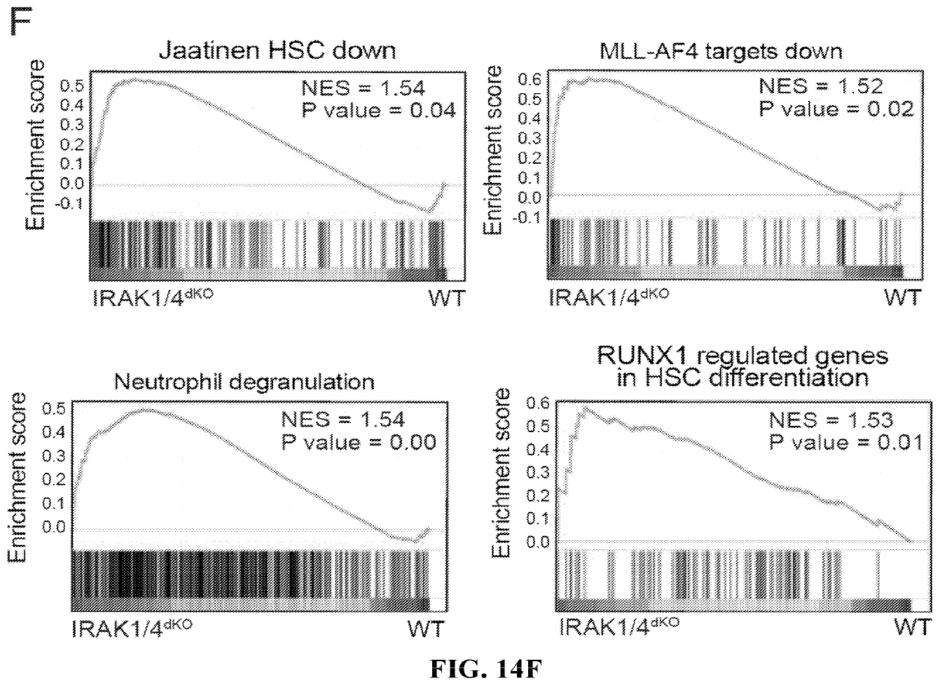

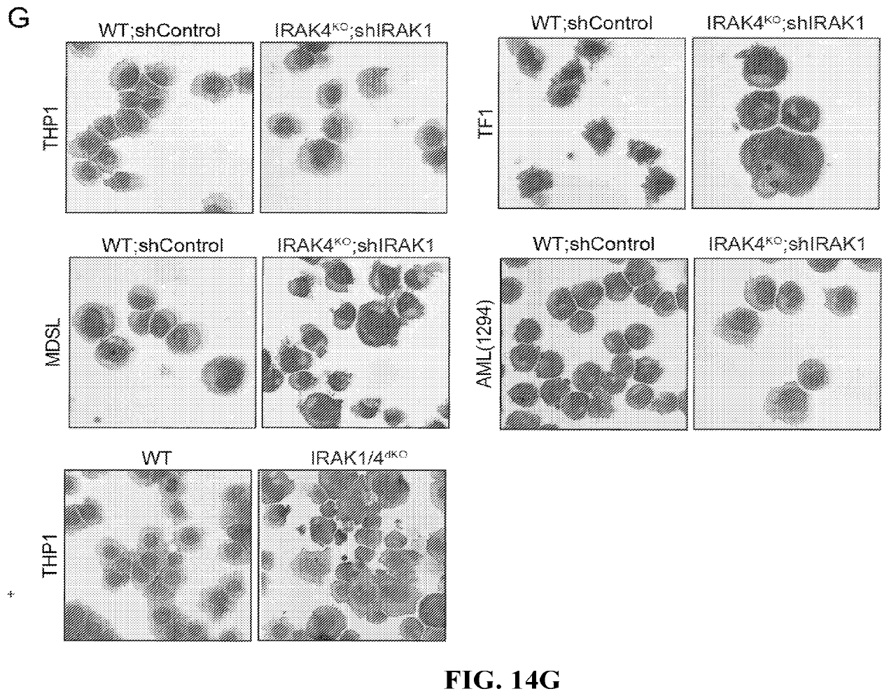

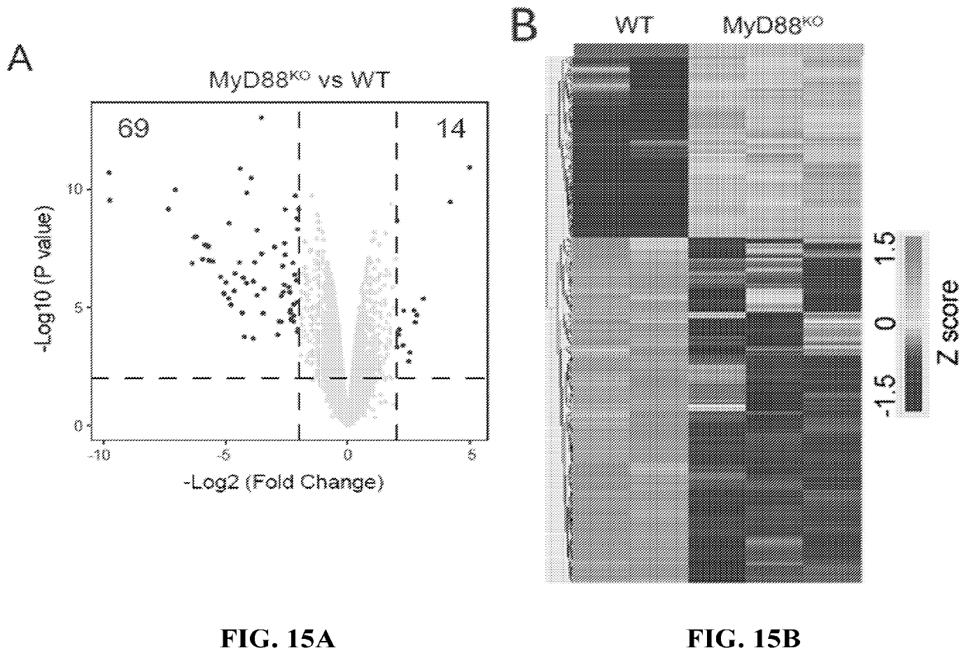

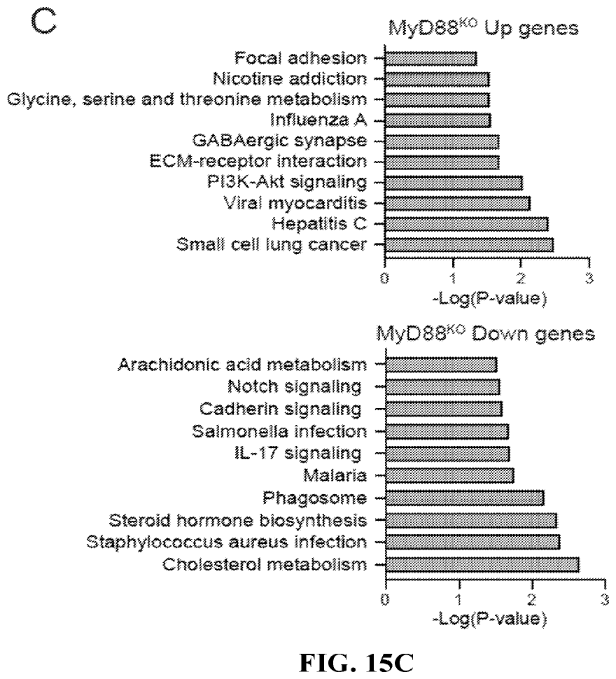

To delineate the gene expression programs that are regulated by non-canonical MyD88-independent IRAK1/4 signaling in leukemic cells, a gene expression analysis was performed in isogenic WT, MYD88^{KO}, IRAK1^{KO} IRAK4^{KO} and IRAK1/4^{dKO} THP1 cells. The replicates from each isogenic cell line clustered together and affirmed distinct gene expression programs (FIG. 14A). To focus on MyD88-independent gene expression programs, all differentially expressed genes that overlapped with the MyD88^{KO} cells were excluded from subsequent analyses (FIGS. 15A-15B and Table 6 in Appendix A). Deletion of IRAK1 or IRAK4 resulted in 129 and 548 repressed genes, respectively, and 98 and 636 overexpressed genes, respectively, versus WT cells (>2-fold change; P value <0.05) (FIG. 14B). Combined deletion of IRAK1 and IRAK4 resulted in 614 downregulated and 720 upregulated genes (FIG. 14B). Examination of the differentially expressed genes (DEGs) revealed a subset of genes unique to IRAK1^{KO}, IRAK4K°, and IRAK1/4^{dKO} cells (FIGS. 14C-14D and Table 7 in Appendix A). The DEG in the IRAK1/4^{dKO} THP1 cells mostly overlapped with the IRAK4^{KO} THP1 cells, with small subsets of genes that were coordinated by IRAK1 (FIGS. 14C-14D). Correspondingly, KEGG and PANTHER pathway analysis of the DEG in the isogenic cell lines corresponded with enrichment of dysregulated pathways in IRAK1^{KO}, IAK4^{KO} and IRAK1/4^{KO} AML cells (FIG. 14E). For example, down regulated genes in IRAK1^{KO} THP1 cells exhibit enrichment of pathways related HIF1a, metabolism, and inflammatory signaling; IRAK4^{KO} THP1 cells exhibit enrichment of pathways related TLR and WNT signaling, and glycolysis; and IRAK1/4^{dKO} THP1 cells exhibit enrichment of pathways related AMPK activation, H1F1a signaling, inflammation, and hematopoietic stem cells (FIG. 14E). MyD88^{KO} THP1 did not exhibit enrichment of these pathways, further supporting the observations that IRAK1/4 signaling can function independent of canonical MyD88 (FIG. 15C). Gene set enrichment analysis (GSEA) revealed that upregulated genes in IRAK1/4^{dKO} cells are associated with loss of HSC states and suppression of MLL-rearranged leukemia, as well as genes that are associated with neutrophil activation and RUNX1-mediated myeloid differentiation (FIG. 14F). A such, targeting of RAK1 and IRAK4 results in gene expression programs associated with LSPC differentiation.

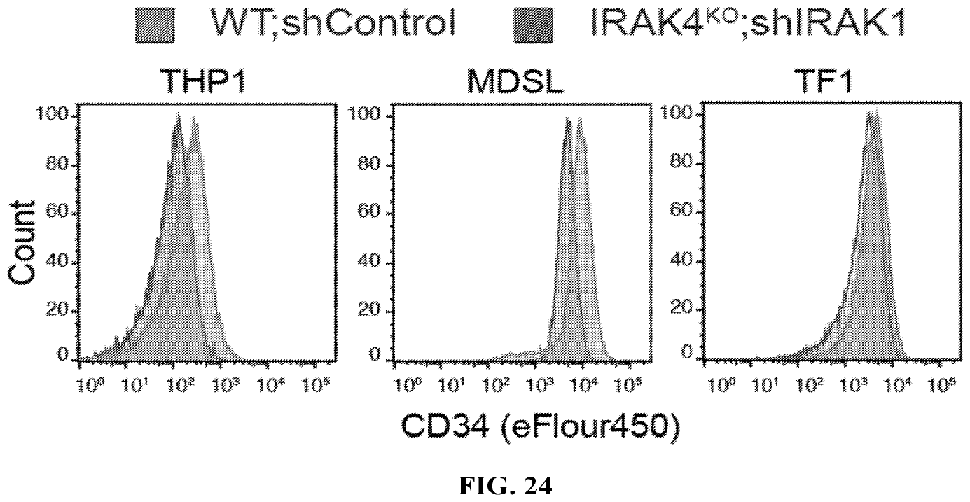

To determine whether IRAK1 and IRAK4 signaling in LSPCs is required for preserving an undifferentiated cell state of LSCPs, morphological changes of MDS and AML cells were examined upon deletion of IRAK1 and IRAK4. Wright-Giemsa staining of isogenic cell lines and patient-derived samples that are proficient (WT) or deficient for IRAK1 and IRAK4 (IRAK4^{KO}; shIRAK1) was performed. As expected, WT MDS/AML cells exhibited uniform morphologies and low cytoplasmic to nuclear ratios (FIG. 14G). In contrast, IRAK1/4 dual-deficient MDS/AML cells had heterogeneous morphologies and size and exhibited increased cytoplasmic to nuclear ratios (FIG. 14G) and decreased expression of CD34 (FIG. 24). Such morphological changes are congruent with myeloid differentiation of leukemic cells. The single IRAK1 or IRAK4 deficient MDS/AML cells also exhibited evidence of morphological changes congruent with differentiation of leukemic cells but not to the magnitude observed upon combined deficient of IRAK1 and IRAK4 (data not shown). Moreover, aberrant myeloid cell differentiation was not observed in MyD88^{KO} MDS and AML cells (FIG. 15D), suggesting that the observed morphological changes of leukemic cells upon deletion of IRAK1 and IRAK4 are uncoupled from canonical MyD88 signaling. These findings suggest that IRAK1 and IRAK4 mediate MyD88-independent functions that are critical for LSPC function by preserving an immature cell state.

IRAK1 and IRAK4 Interactomes Reveal Non-Canonical Signaling Networks Associated with LSPC States.

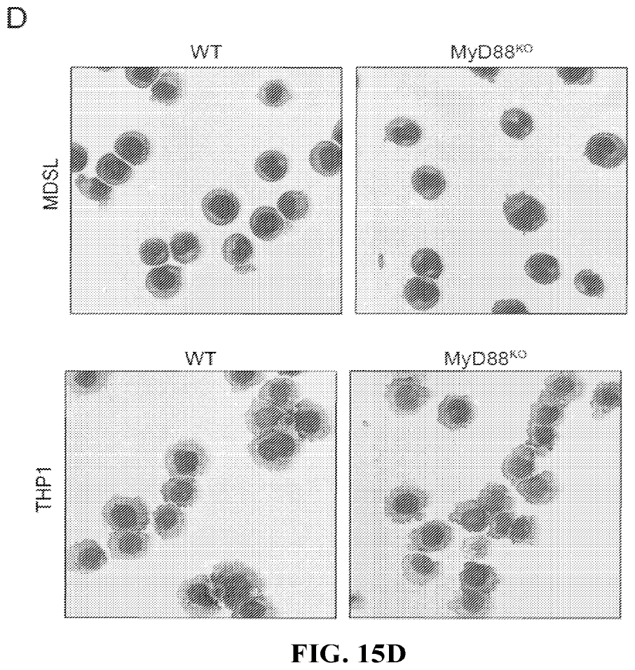

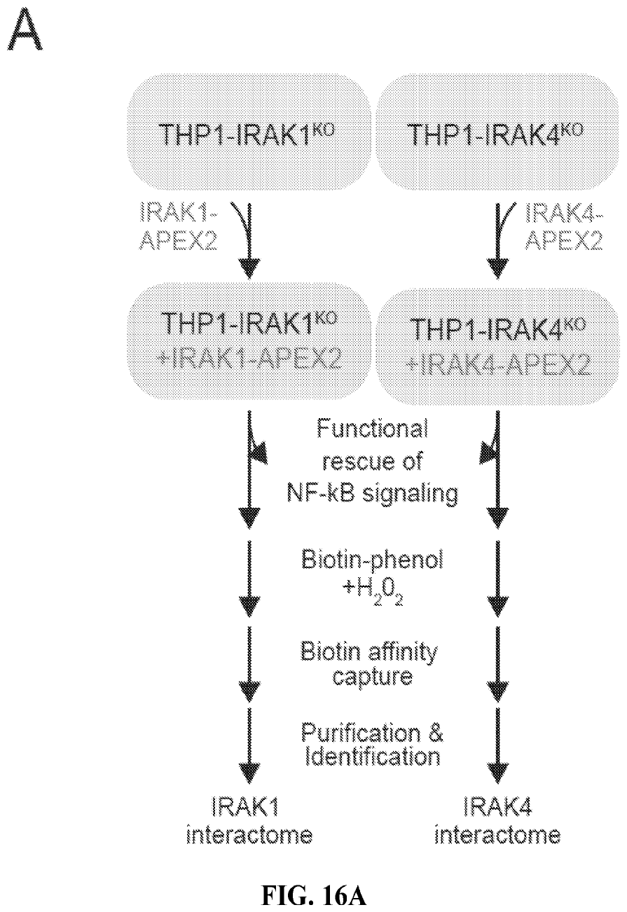

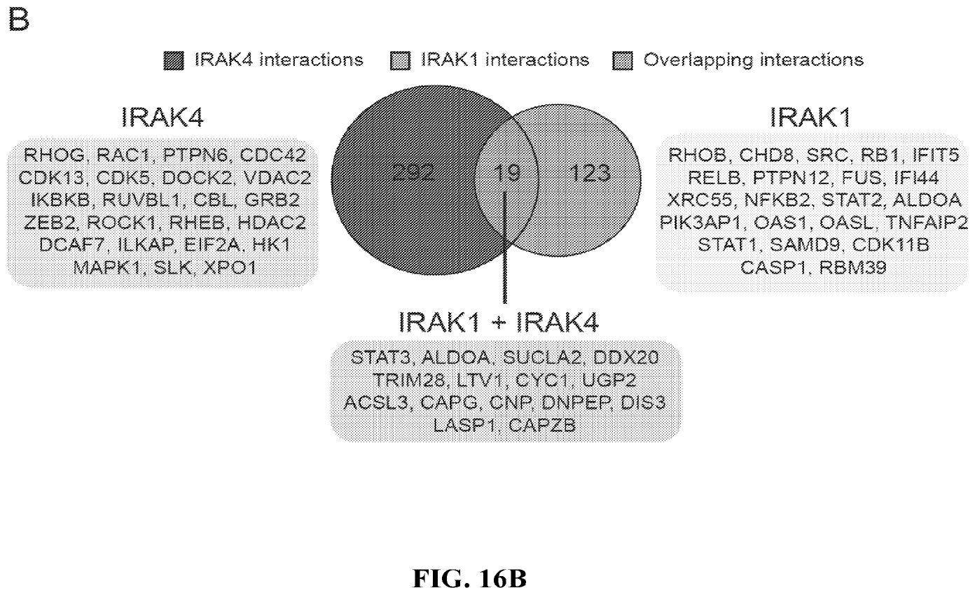

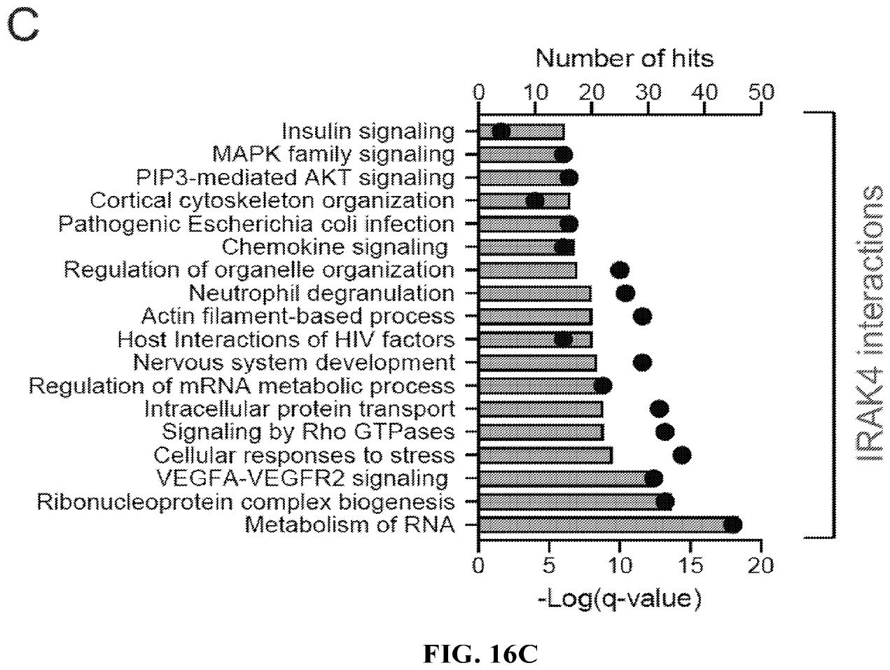

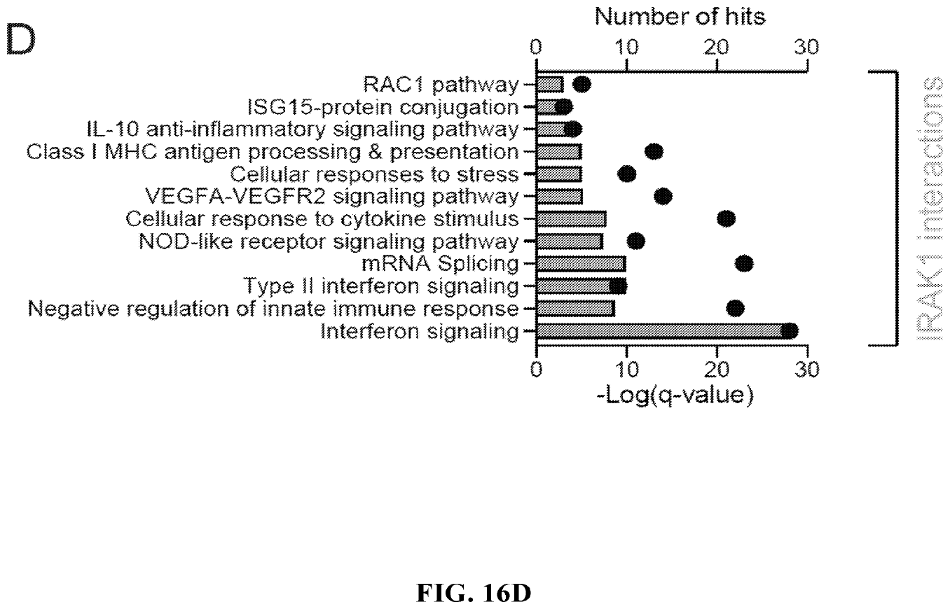

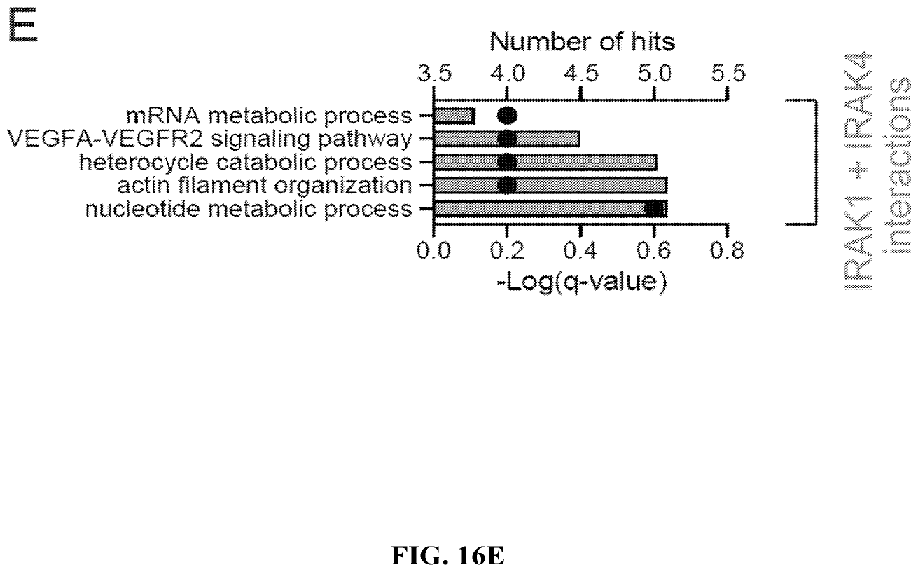

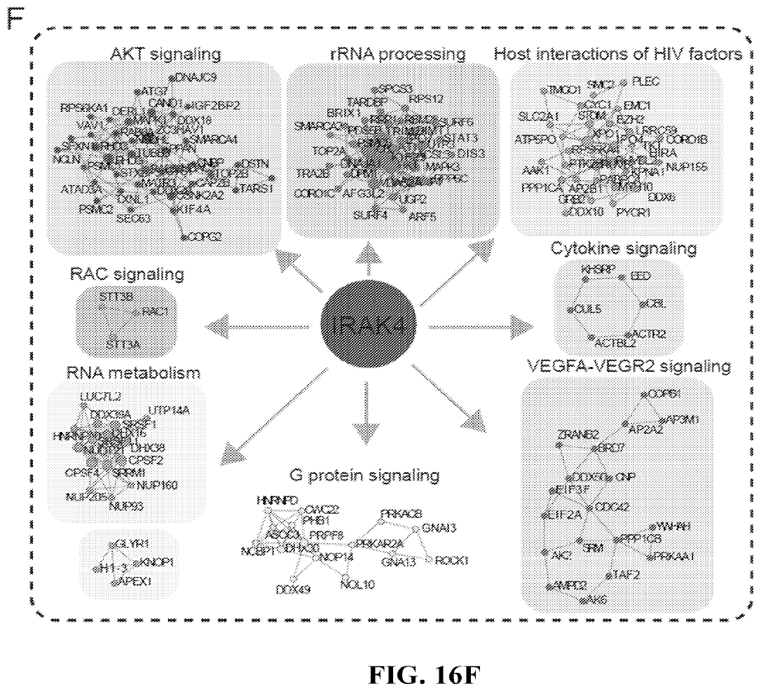

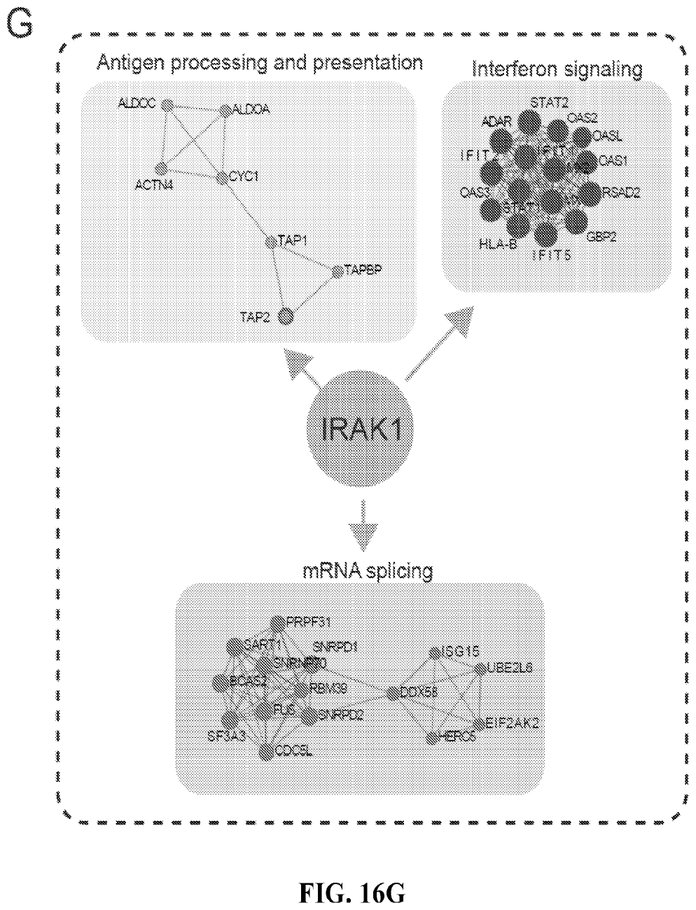

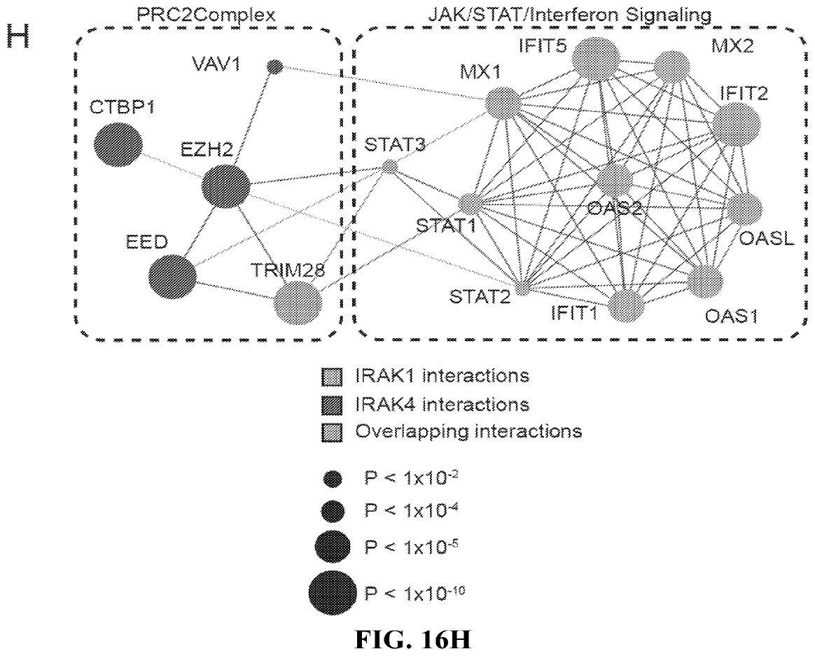

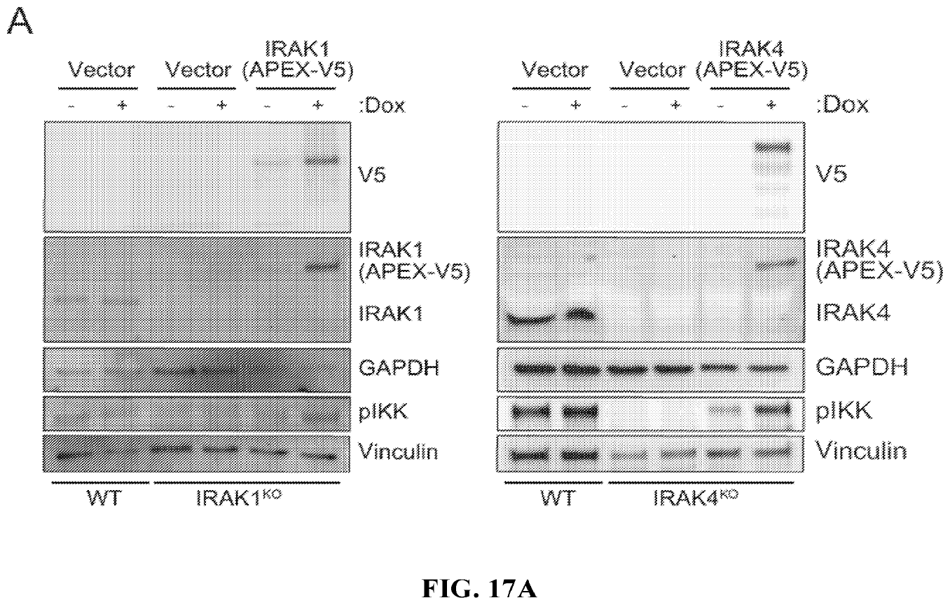

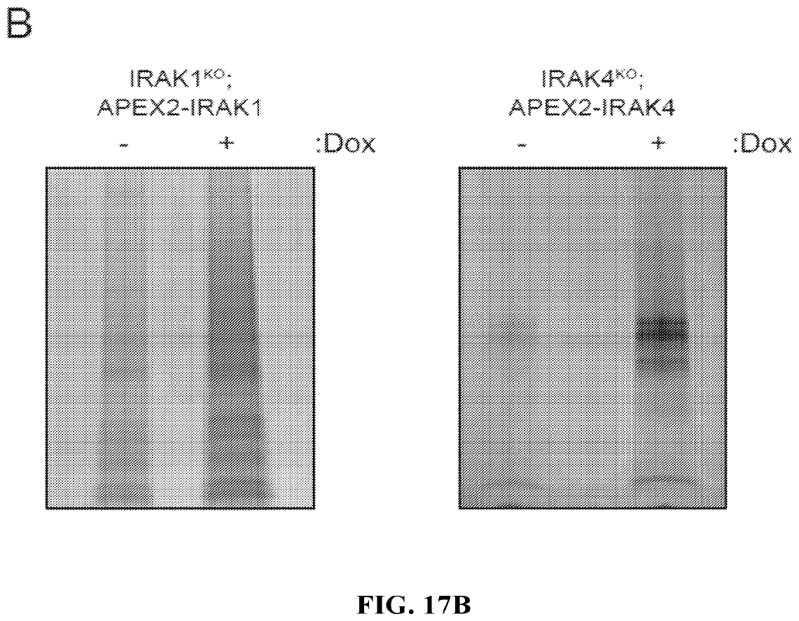

It was found that IRAK1 and IRAK4 coordinate complimentary gene expression programs implicated in maintaining the undifferentiated state of LSPCs; however, the non-canonical signaling mechanisms and key effectors by which IRAK1/4 regulate these programs remains unknown. As such, to map out the IRAK1 and IRAK4 interactome in AML proximity labeling was performed followed by mass spectrometry analysis using APEX2. In the presence of biotin-phenol and hydrogen peroxide, APEX2 labels proteins with biotin in a 20 nm radius. APEX2 was fused to the N-terminal domains of V5-tagged IRAK1 and IRAK4 and expressed individually under the control of a doxycycline-inducible promoter (pCW57.1GFP) in IRAK1^{KO} and IRAK4^{KO} THP1 cells, respectively (FIG. 16A). Doxycycline-induced expression of IRAK1- and IRAK4-APEX2 fusion constructs was confirmed in IRAK1^{KO} (IRAK1^{KO}; APEX2-IRAK1) and IRAK4^{KO} (IRAK4^{KO}; APEX2-IRAK4) THP1 cells by immunoblotting with antibodies against V5 and either IRAK1 or IRAK4 (FIG. 17A). It was also confirmed that APEX2-IRAK1 and APEX2-IRAK4 retained their normal functions as they rescued the NF-kB signaling defect in IRAK1^{KO} and IRAK4^{KO} THP1 cells, respectively (FIG. 17A). Proximity protein labeling was performed by inducing the APEX2 fusion proteins in IRAK1^{KO}; APEX2-IRAK1 and IRAK4^{KO}; APEX2-IRAK4 THP1 with doxycycline, incubating with biotin-phenol, and activating the APEX2 enzyme with hydrogen peroxide (FIG. 17B). Biotin tagged proteins from lysed cells were precipitated for identification and quantitative analysis on the LTQ-Orbitrap Elite Mass Spectrometer. Unique peptides that were >2-fold enriched in the doxycycline-treated samples (false discovery rate adjusted label-free quantification P value <0.05) were pursued. Based on this criterion, 311 proteins proximal to IRAK4 and 142 proteins proximal to IRAK1 were identified in THP1 cells (FIG. 16B and Tables 8 and 9 in Appendix A). Two hundred ninety-two proteins were unique to IRAK4 and 123 were unique to IRAK1 (FIG. 16B), while 19 proteins were common to IRAK1 and IRAK4 (FIG. 16B).

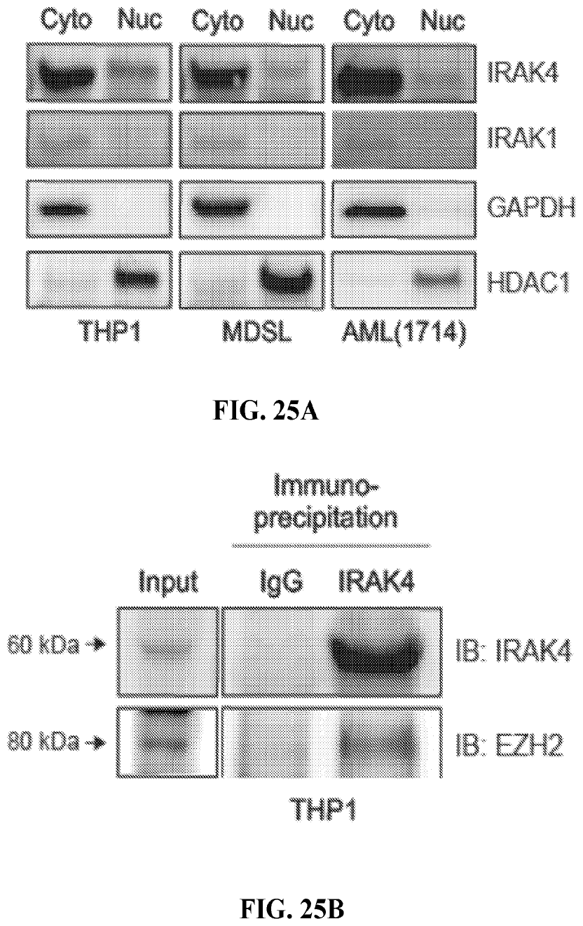

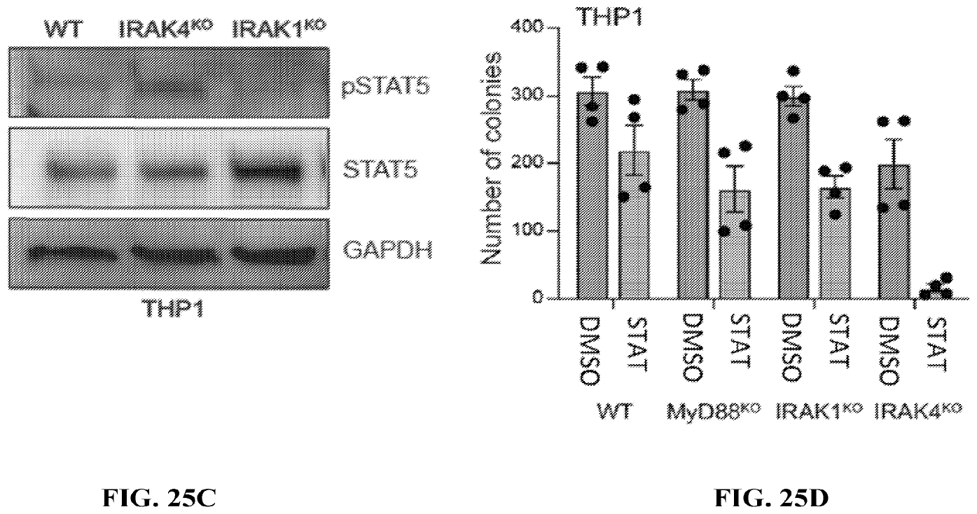

Canonical IRAK1/4 signaling activates members of the NF-kB and MAPK pathways. As expected, several proteins implicated in functions associated with canonical IRAK1/4 signaling were identified, such as IKKb (IKBKB), RELB, p100/NFKB2, and ERK (MAPK1) (FIG. 16B). To uncover novel IRAK1/4 signaling networks in AML, an ontology pathway analysis was performed with Metascape using the list of IRAK1 (n=142) and IRAK4 (n=311) proximal proteins. The pathway analysis revealed that proximal IRAK4 proteins include effectors of multiple signaling pathways, such PI3K/AKT (GRB2, ERK1/2, GRB2, CBL), MAPK (ERK1 and ERK2), and RHO/GTPases (RAC1, VAV1, CDC42, and ROCK1) (FIG. 16C). Importantly, the dysregulated genes observed in IRAK4^{KO} AML (FIG. 14E) are enriched for several pathways, such as signaling related to integrins, inflammation, and TLRs, that can be directly explained by the aforementioned IRAK4 proximal proteins. Proximal IRAK1 proteins were significantly enriched for effectors of interferon and antiviral signaling (RIG-1, MX1, MX2, OAS1, STAT1 and STAT3) (FIG. 16D). Since STAT3 cooperates with HIF-1a in mediating target gene expression, STAT1/3 regulation by IRAK1 may therefore provide a mechanistic basis for the observed dysregulation of HIF-1a target genes in IRAK1^{KO} AML (FIG. 14E). There were also proteins that were found proximal to both IRAK1 and IRAK4, which include effectors of interferon/viral, VEGFR signaling and mRNA metabolism (FIG. 16B and FIG. 16E). The enriched pathways among IRAK1 and IRAK4 proximal proteins are predicted to form physical interactions as demonstrated by a protein-protein interaction enrichment analysis using STRING (FIGS. 16F-16G). The IRAK1 and IRAK4 proximal proteins were next focused on as well as pathways that may provide a mechanistic explanation for the aberrant expression of genes that contribute to LSPC differentiation upon targeting of IRAK1/4. Integration of the enriched pathways derived from the gene expression analysis (FIGS. 16A-16H) and the interactome analysis highlighted the potential convergence of IRAK1 on interferon-related signaling and IRAK4 on the heteromeric polycomb repressive complex 2 (PRC2) (FIG. 16H). As indicated above, several IRAK1 proximal proteins were identified that were directly implicated in interferon receptor and JAK-STAT signaling (FIG. 16H). Additionally, several members of the PRC2 core (EZH2, EED, CTBP1, VAV1) were identified among the top-ranking IRAK4 proximal proteins (FIG. 16H). SUZ12, EED, and EZH2 are part of the core heteromeric PRC2 complex, a chromatin regulator that is responsible for repression of target genes by methylating H3K27. JAK-STAT and PRC2 function, including activating EZH2 mutations, are well-defined in AML LSPCs by regulating genes that promote cellular differentiation. Since the predicated IRAK1 and IRAK4 interactions are with nuclear proteins, the cellular localization of IRAK1 and IRAK4 in leukemic cells was examined. IRAK4 localized to the cytoplasm and nucleus of MDS and AML cells (FIG. 25A). In contrast, IRAK1 was primarily localized to the cytoplasm in these cells. To determine whether IRAK4 directly associates with the PRC2 complex, co-immunoprecipitation experiments were performed in AML cells. It was found found that EZH2 co-immunoprecipitated with IRAK4 in THP1 cells (FIG. 25B), suggesting that nuclear IRAK4 can directly associate with the PRC2 complex in AML. To further interrogate this proposed IRAK1-IRAK4 network, it was also determined whether deletion of IRAK4 results in a corresponding increase in IRAK1-dependent JAK-STAT signaling. IRAK4^{KO} cells exhibited increased phosphorylated STAT5 as compared to WT cells, while IRAK1^{KO} cells expressed phosphorylated STAT5 below the levels observed in WT cells (FIG. 25C). Moreover, IRAK4^{KO} THP1 cells were sensitive to a STAT inhibitor (BB1608) as compared to IRAK1^{K}, MyD88^{KO}, or WT cells (FIG. 25D). Collectively, these findings reveal extensive IRAK1 and IRAK4 signaling networks implicated in maintaining LSPCs and that extend beyond canonical MyD88-dependent signaling.

IRAK1/4 maintains undifferentiated leukemic cell states through chromatin and transcription factor networks

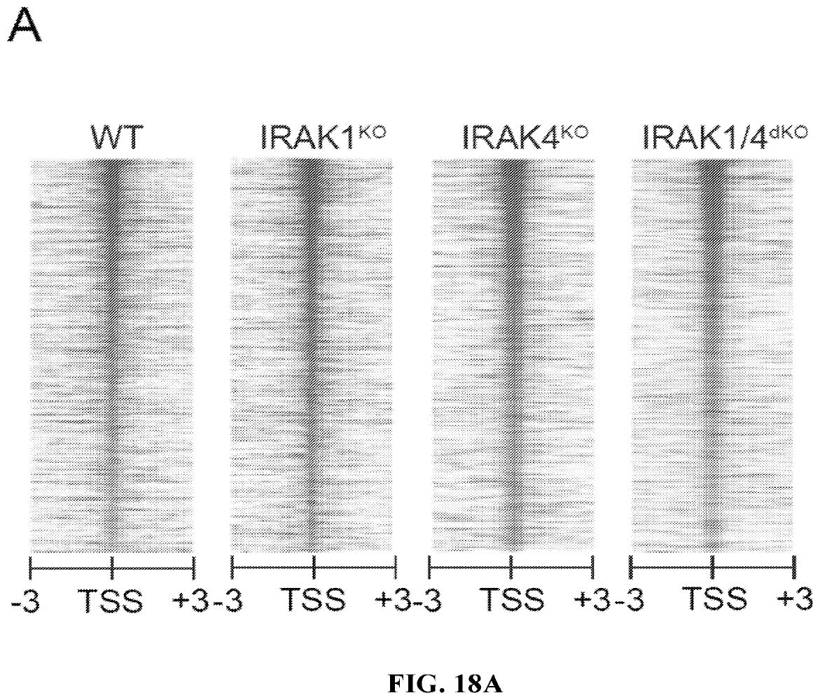

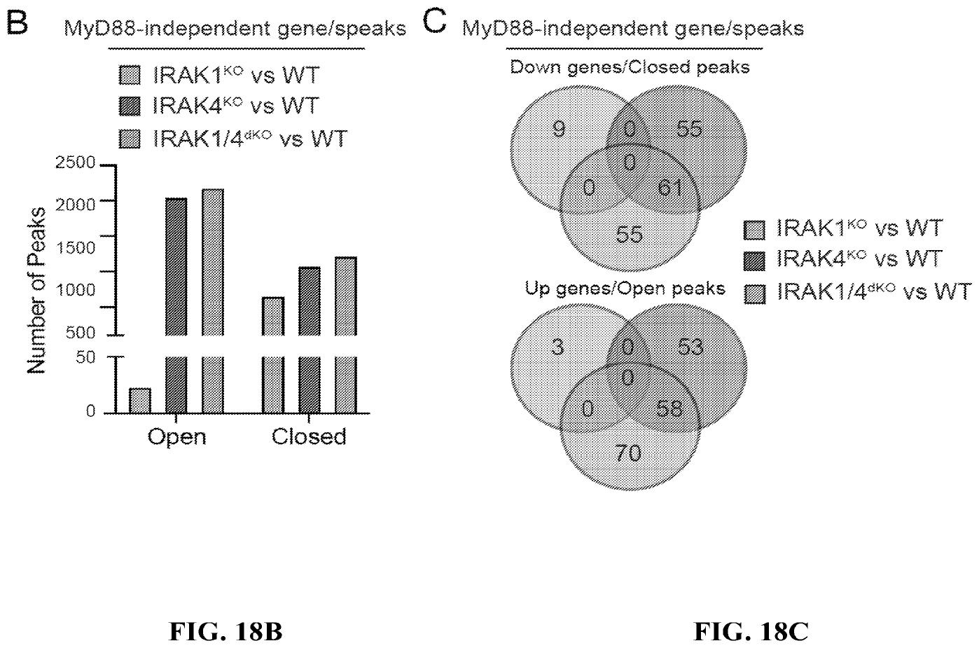

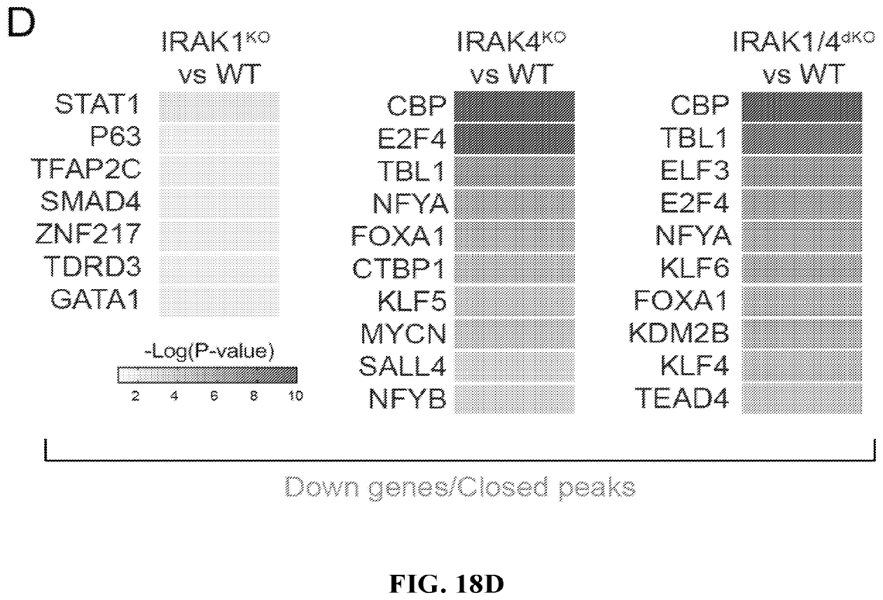

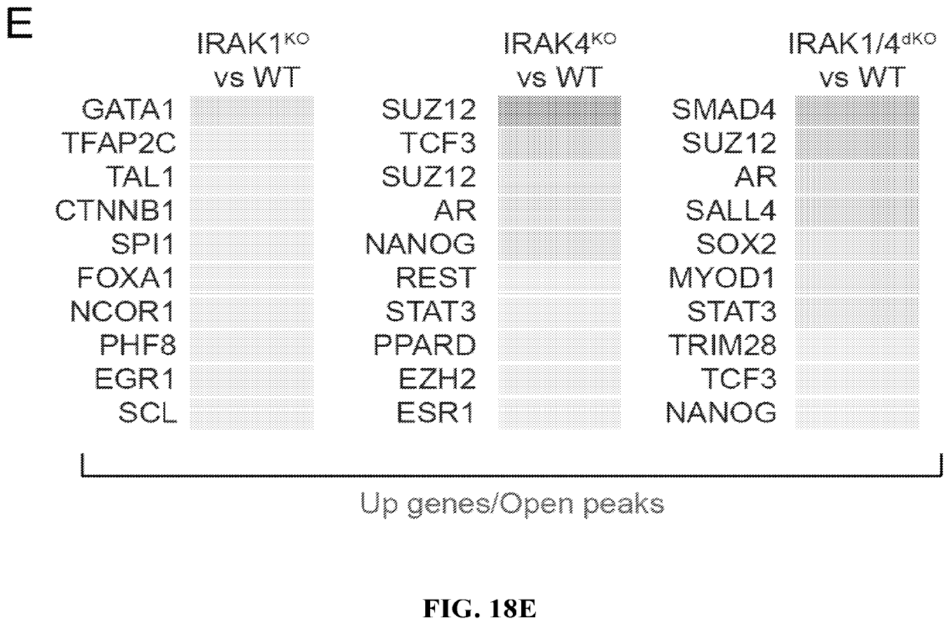

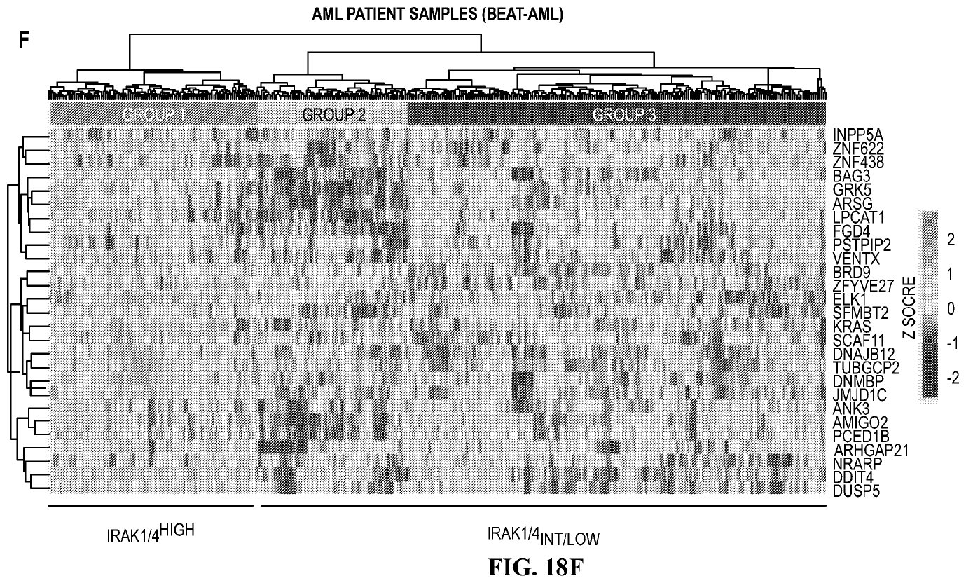

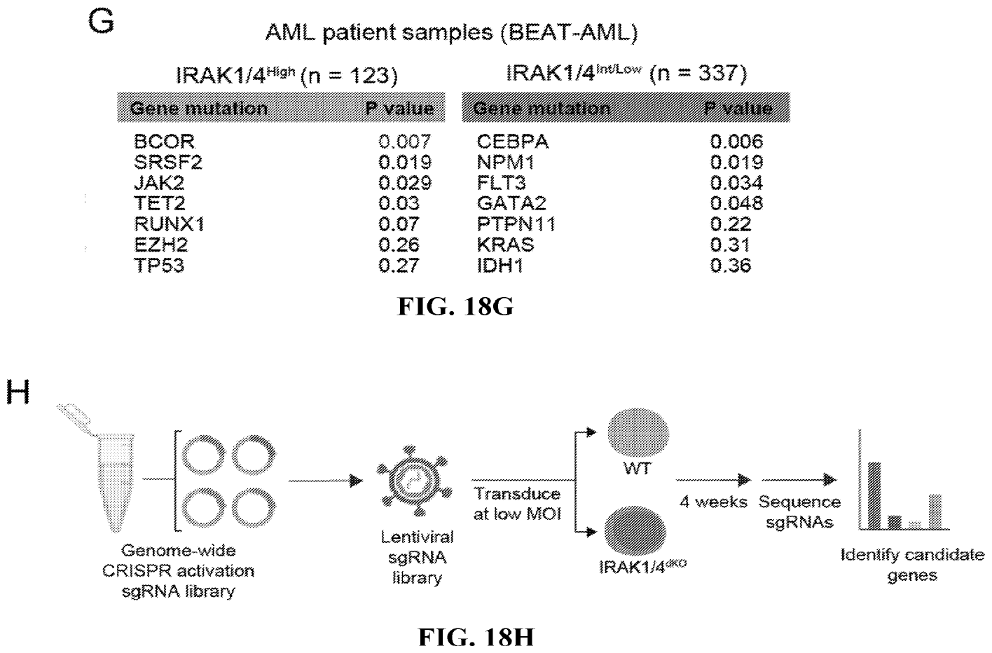

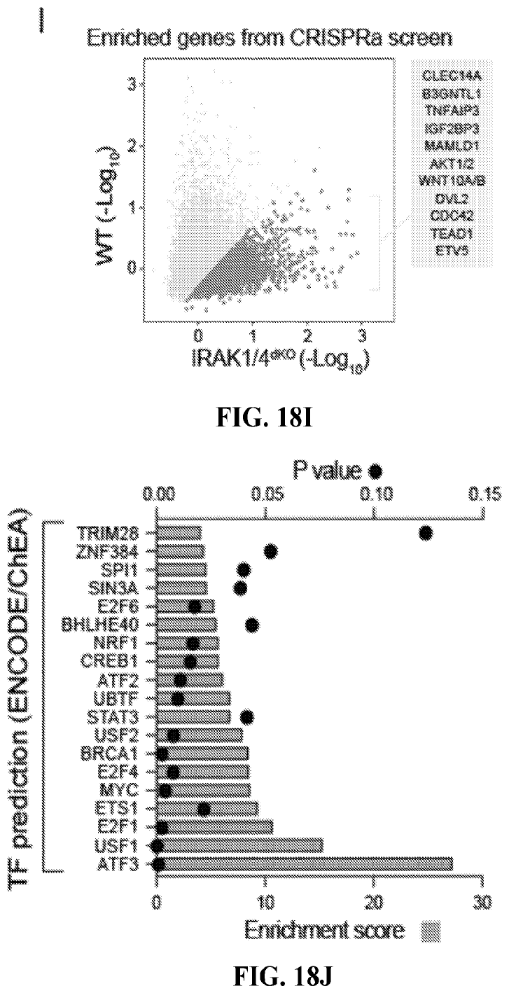

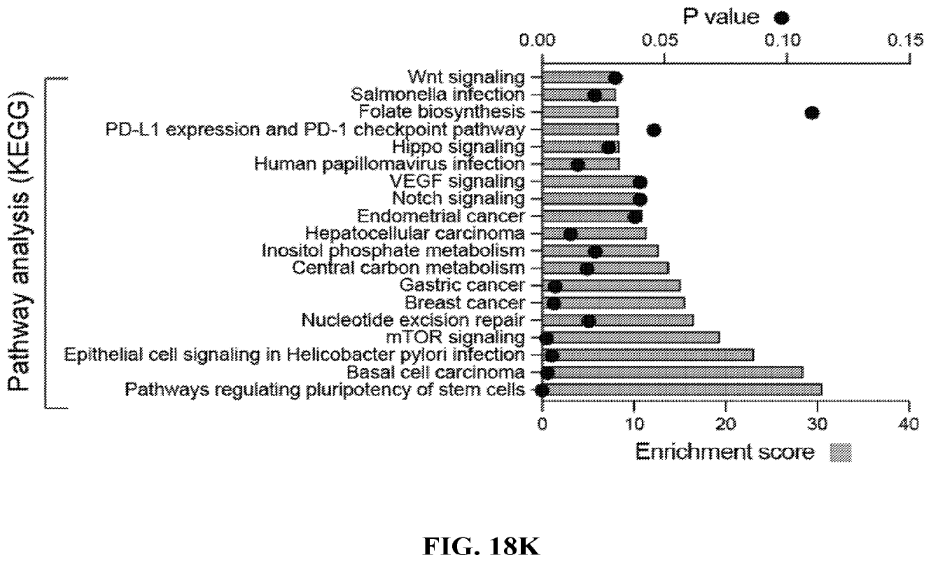

Next, studies were performed to gain further insight into the regulatory effectors downstream of non-canonical IRAK1/4 signaling that are required for maintaining LSPC states. Although the proteomic and transcriptomic analyses implicated several relevant pathways in MDS/AML, how IRAK1/4 signaling may coordinate chromatin and transcriptional factor networks was the focus. The PRC2 complex was nominated as one of the top-ranking hits among the IRAK4 proximal proteins and is responsible for transcriptional gene regulation in LSPCs via H3K27 tri-methylation (me3) at select genomic locations. One consequence of PRC2-mediated H3K27me3 is compaction of chromatin and loss of transcriptional factor accessibility at select gene loci, as such, chromatic accessibility was studied by performing Transposase-Accessible Chromatin using sequencing (ATAC-seq) and mapped global changes in chromatin accessibility in isogenic WT, MYD88^{KO}, IRAK1^{KO}, IRAK4^{KO}, and IRAK1/4^{dKO} THP1 cells (FIG. 18A). To prioritize MyD88-independent pathways, as above, all differential chromatin peaks identified in MYD88^{KO} THP1 cells were excluded (Table 10 in Appendix A). IRAK1^{KO} (n=657), IRAK4^{KO} (n=1083), and IRAK1/4^{dKO} (n=1224) THP1 cells displayed a similar number of closed peaks (FIG. 18B and Table 11 in Appendix A). However, IRAK1^{KO} THP1 cells exhibited significantly fewer open peaks (n=23) as compared to IRAK4^{KO} (n=2051) and IRAK1/4^{dKO} (n=2184) THP1 cells (FIG. 18B). Upon examination of gene loci proximal to the differential chromatin peaks (<1 Mbp; P<0.01), genes associated with increased chromatin accessibility peaks were identified (IRAK1=21, IRAK4=1524, and IRAK1/4=1705), as well as genes associated with loss of accessibility peaks (IRAK1=525, IRAK4=792, and IRAK1/4=824) in IRAK1^{KO}, IRAK4^{KO} and IRAK1/4dKO THP1 cells (FIG. 18C). IRAK4-regulated chromatin accessibility peaks are preserved in IRAK1/4^{dKO} THP1 cells, while the effects of RAK1 on chromatin accessibility is minimal (FIG. 18C). Of the 525 genes associated with loss of chromatin peaks in IRAK1^{KO} THP1 cells, only 9 genes were significantly downregulated in the RNA sequencing data set (FIG. 18C), which are predicted targets of STAT1 (P=0.002) (FIG. 18D).

To identify IRAK4-regulated factors that drive the IRAK1/4^{dKO} AM/L cell phenotype, genes that are both downregulated and associated with loss of chromatin accessibility in IRAK4^{KO} and IRAK1/4^{dKO} THP1 cells were first assessed (FIG. 18C). Significant overlap of genes that are repressed in IRAK4^{Ko} and IRAK1/4^{dKO} THP1 cells was identified, which are enriched as targets of CREB Binding Protein (CBP) (P=2.1×10⁻²¹) and E2F Transcription Factor 4 (E2F4) (P=8.3×10⁻¹¹) (FIG. 18D). CBP and E2F4 are both implicated in maintaining LSPCs^{34,35}. Next, genes associated with open chromatin peaks and upregulated expression that are conserved in IRAK4^{KO} and IRAK1/4^{dKO} THP1 cells were examined (FIG. 18C). This set of genes were enriched for regulatory targets of SUZ12 (P=1.4×10⁻⁵), EZH2 (P=0.04), STAT3 (P=0.01), and NANOG (P=0.006) (FIG. 18E). Notably, SUZ12 and EZH2 are core members of the PRC2 complex. Thus, based on the transcriptomic and proteomic analyses, deletion of IRAK1 and IRAK4 is associated with loss of JAK-STAT signaling and PRC2-mediated function, and consequently expression of genes that mediate myeloid leukemia cell differentiation. These findings suggest that IRAK4 regulates a broad network of transcription factors and gene expression programs, while RAK1 impacts a distinct and restricted gene regulatory network, that collectively preserve LSPC function. These observations also support the concept that IRAK1 and IRAK4 function in a non-redundant mechanism independent of MyD88 signaling.

### IRAK1/4 Signaling Programs Define a Subset of MDS and AML Patients

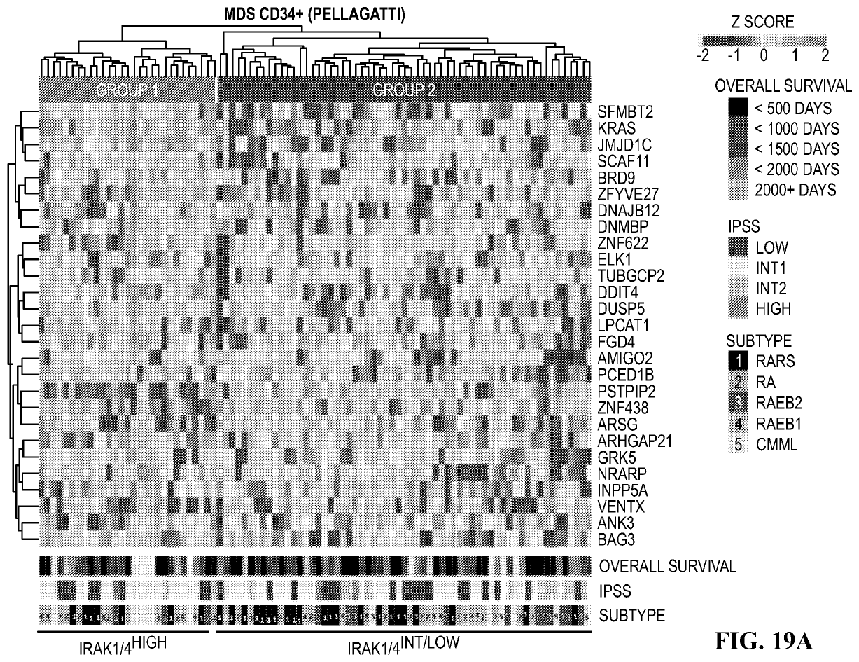

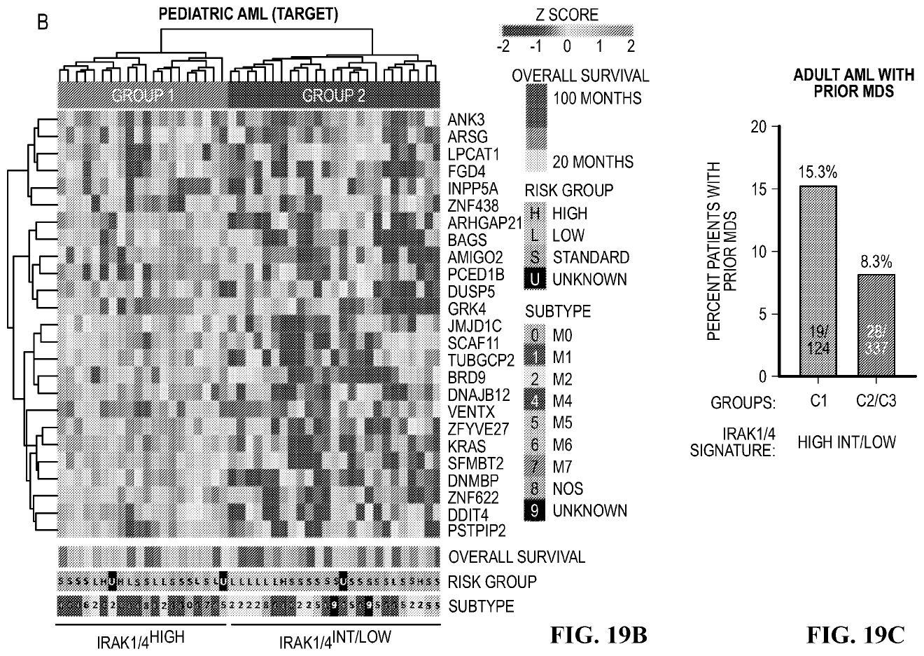

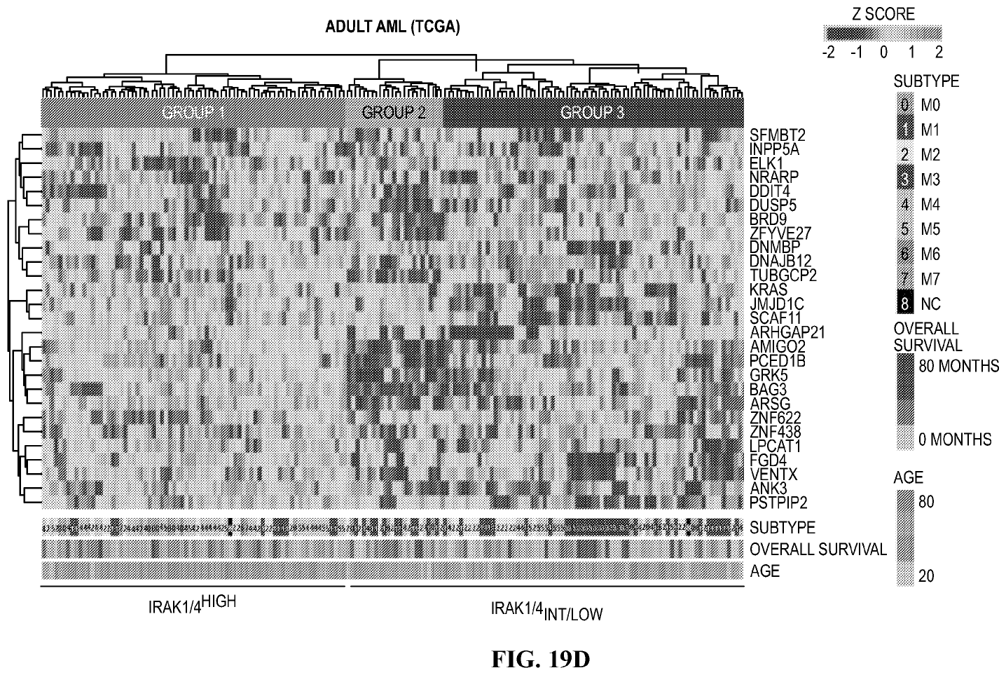

To establish whether the core IRAK1/4 signaling program is operational in AML patients, an unsupervised hierarchical clustering analysis of RNA sequencing data from the BEAT AML trial for differential expression of the defined IRAK1/4-associated genes was performed. The set of 51 genes that were downregulated and associated with loss of chromatin accessibility in IRAK1/4^{dKO} THP1 cells was utilized. The list of genes was restricted to ones that were also differentially expressed in AML versus healthy controls (>2-fold change; P<0.05) (n=27 genes; Table 12 in Appendix A). Patients with AML from the BEAT AML data set revealed 3 groups characterized by the distinct expression of the IRAK1/4-associated genes at diagnosis (FIG. 18F). Group 1 consists of AML patients with elevated expression of the IRAK1/4-associated genes (“IRAK1/4^{High}”), whereas Group 2 and 3 consists of AML patients with decreased and/or variable expression of these genes (“IRAK1/4^{Int/Low}”) (FIG. 18F). IRAK1/4High AML were enriched for mutations in BCOR (P=0.007), SRSF2 (P 0.0019), JAK2 (P=0.029), TET2 (P=0.03), RUNX1 (P=0.07), EZH2 (P=0.27) as determined by Hypergeometric testing (FIG. 18G). In contrast, the same cohort of IRAK1/4^{High} AML patients were significantly underrepresented for mutations in CEBPA (P=0.006), NPM1 (P=0.019), FLT3 (P=0.034), GATA2 (P=0.048) and PTPN11 mutations (P=0.22) (FIG. 18G). These enrichment mutation profiles are consistent with altered chromatin assembly via the PRC1/2 complex (BCOR, SRSF2, EZH2) and JAK-STAT signaling (JAK2). Independent analysis of the IRAK1/4-associated gene signature in additional adult (TCGA) and pediatric (TARGET) AML populations and MDS CD34+ cells also divided the patients into groups based on high and intermediate/low expression of IRAK1/4-associated genes (FIGS. 19B-19D). Although the IRAK1/4-associated gene signature did not have prognostic value nor did it correlate with age, the IRAK1/4 signature did correlate with myelomonocytic subtypes (M4 FAB) of AML in adult (P=3.28×10⁻⁵) and pediatric patients (P=0.013) and with an antecedent MDS (Supplemental FIG. 19D). These findings reveal that IRAK1/4 signaling programs are operational in MDS/AML and could define a subset of adult and pediatric patients with a greater dependency on IRAK1/4.

### IRAK1/4 Deficiency can be Rescued by Reactivation of Stem Cell Programs and Leukemia-Associated Transcriptional Programs

To gain insight into the cellular processes that contribute to attrition of LSPCs following IRAK1/4 inhibition, a genome-scale CRISPR activation (CRISPRa) screen was performed to identify genes that restore the growth ability of IRAK1/4-deficient AML cells. IRAK1/4^{dKO} THP1 cells were selected for the screen as they exhibit a growth defect that correlates with impaired LSPC function in vitro and in vivo. WT and IRAK1/4^{dKO} THP1 cells were transduced with a CRISPRa single-guide RNA (sgRNA) library consisting of sgRNAs activating 18,000 coding isoforms (FIG. 18H). The transduced cells were grown for 3 weeks and then the sgRNA library was deep-sequenced. MAGeCK was performed on the independent replicates to identify candidate genes that were enriched in IRAK1/4^{KO} relative to WT cells (FIG. 18I and Table 13 in the Appendix). Several candidate genes have been previously shown to mediate leukemic activity, such as ETV5, TEAD1, CDC42, AKT1/2, and WNT10A. Pathway analysis on the candidate genes (top 438 based on fold change and P value) identified in IRAK1/4^{dKO} cells revealed significant enrichment of pathways implicated in stem cell activity (NES, 30.5; P value=0.0032; Adjusted P value=0.07) and cancer-related pathways (FIG. 18K). These overexpressed genes are predicted to be transcription targets of ATF3, USF1, E2F1, and MYC (FIG. 18J), which are directly implicated in LSPC function. Moreover, STAT3 target genes enriched in in IRAK1/4^{dKO} cells were identified, supporting the earlier observations that loss of STAT signaling contributes to the impairment of IRAK1/4-deficient AML cells (FIG. 18J). This analysis highlighted that IRAK1/4 inhibition can be rescued by reactivation of stem cell programs and leukemia-associated transcriptional programs.

### a Dual IRAK1/4 Inhibitor is Effective at Suppressing MDS/AML LSPCs

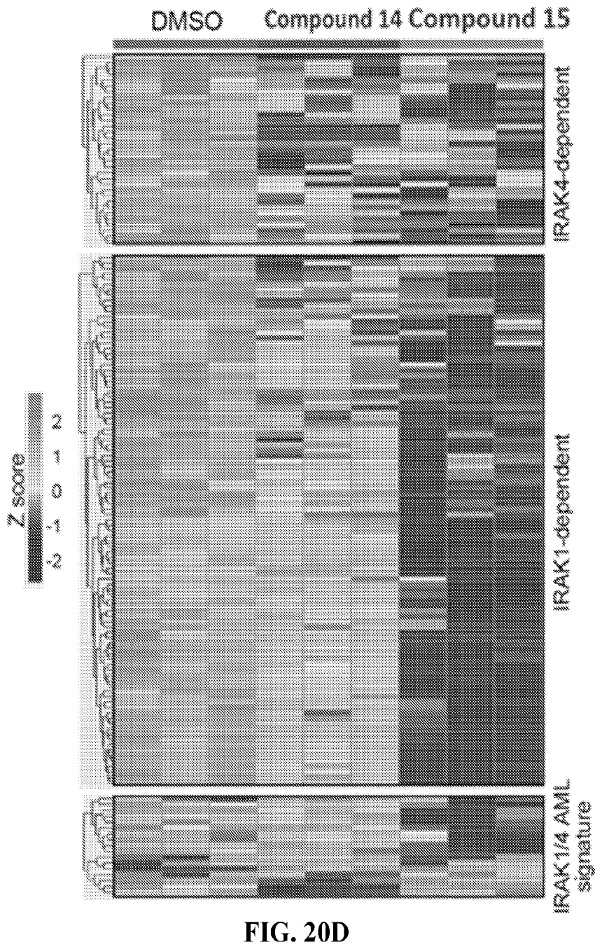

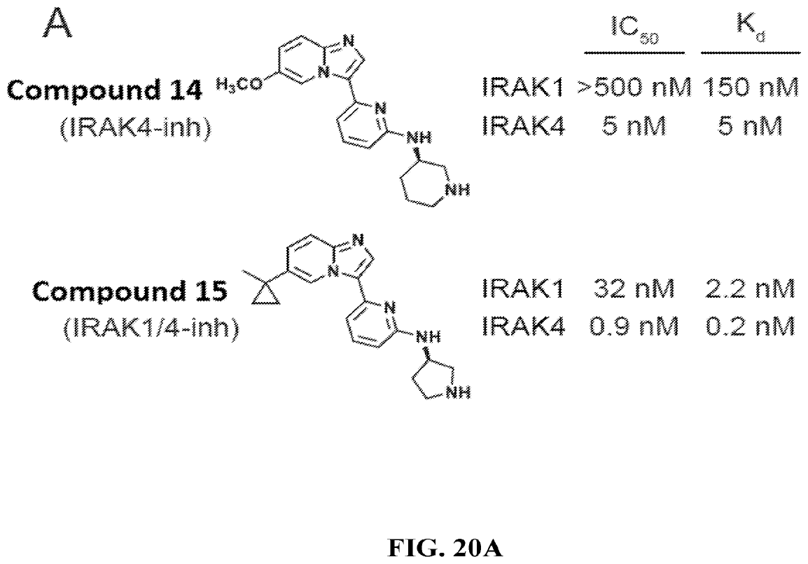

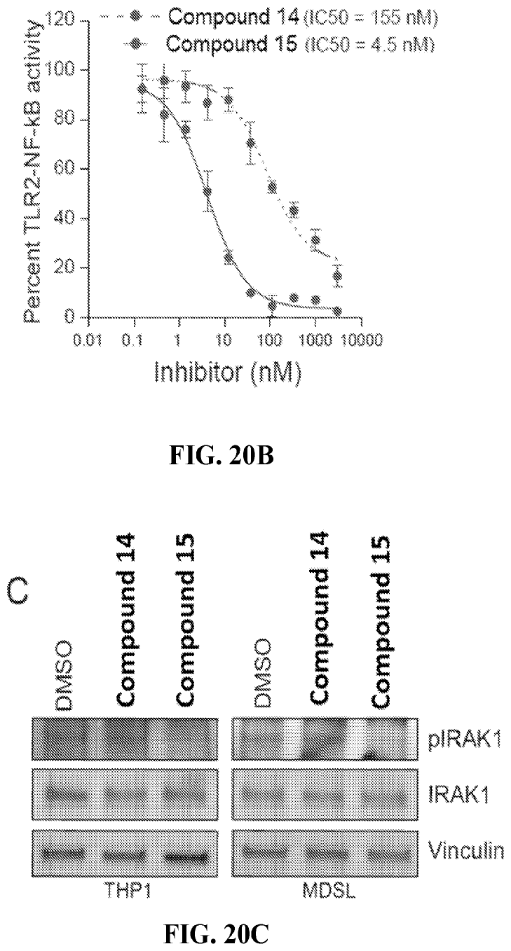

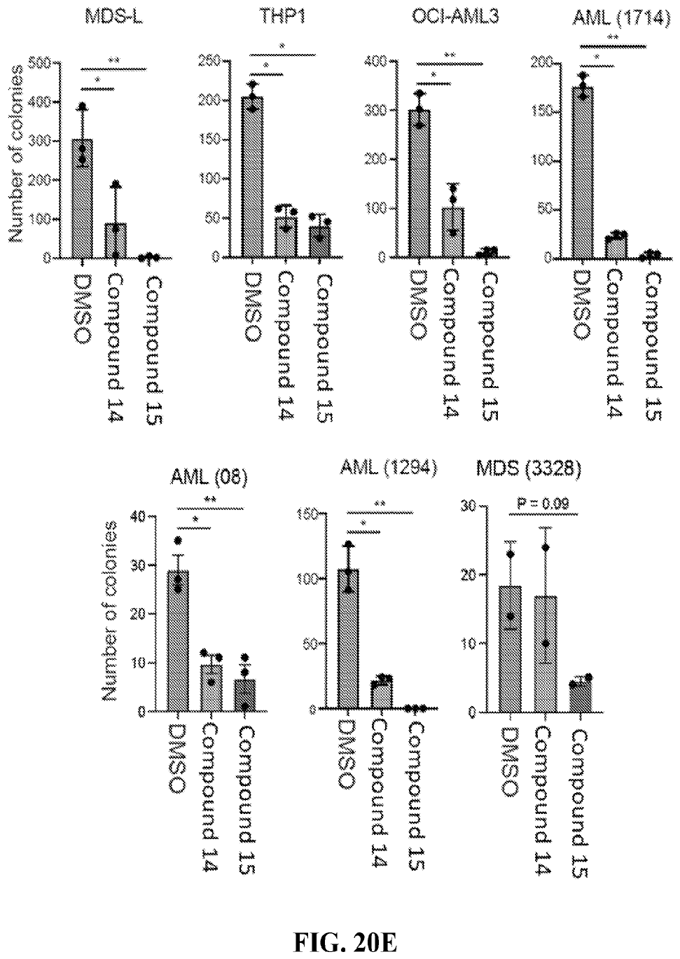

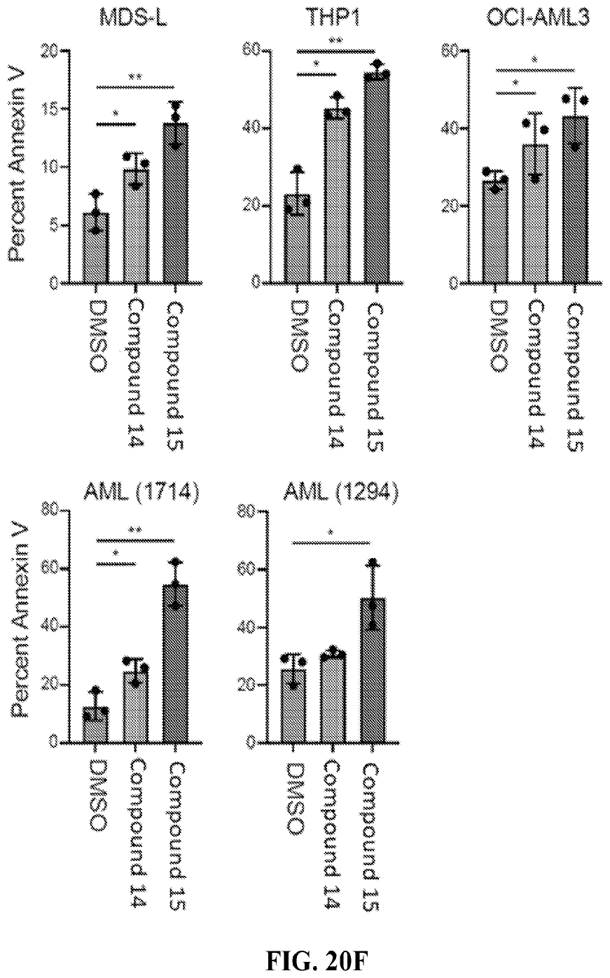

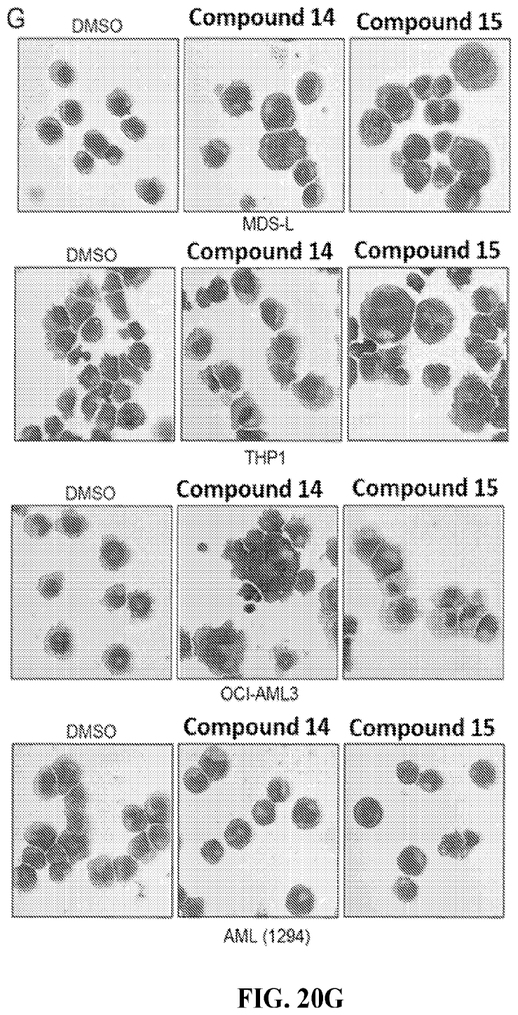

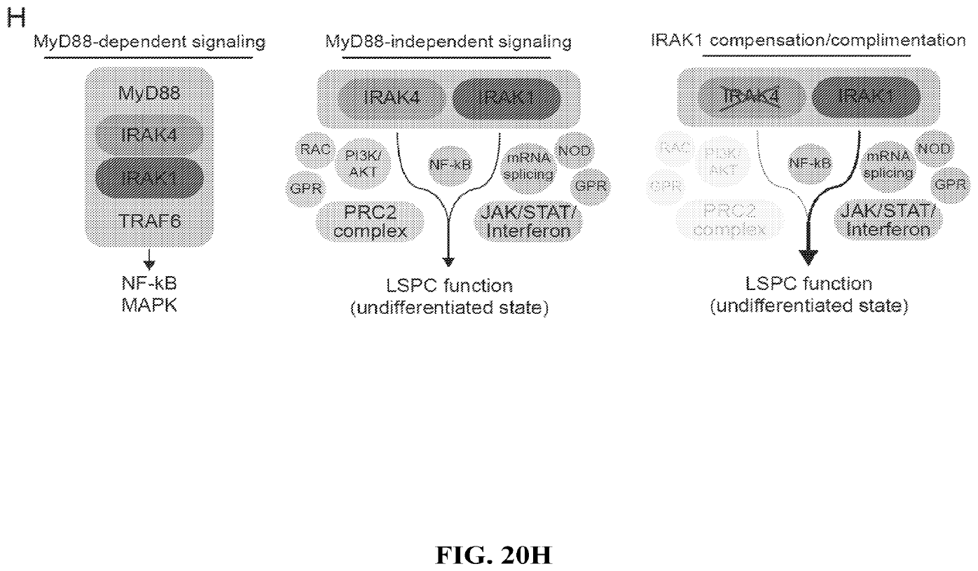

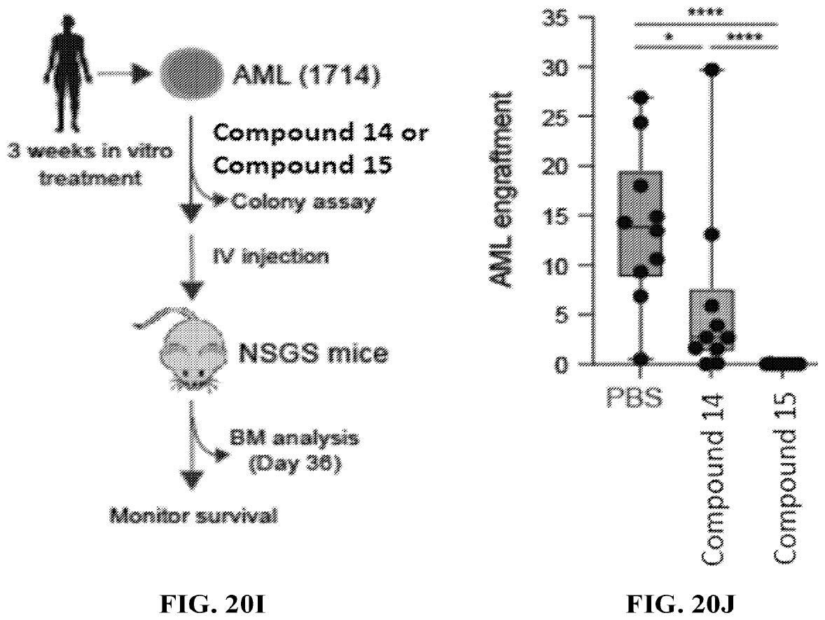

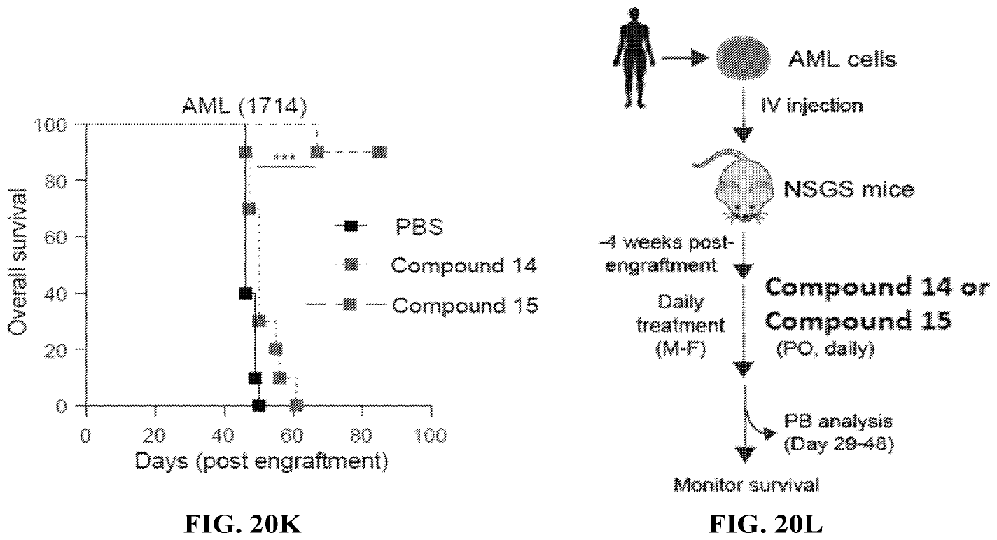

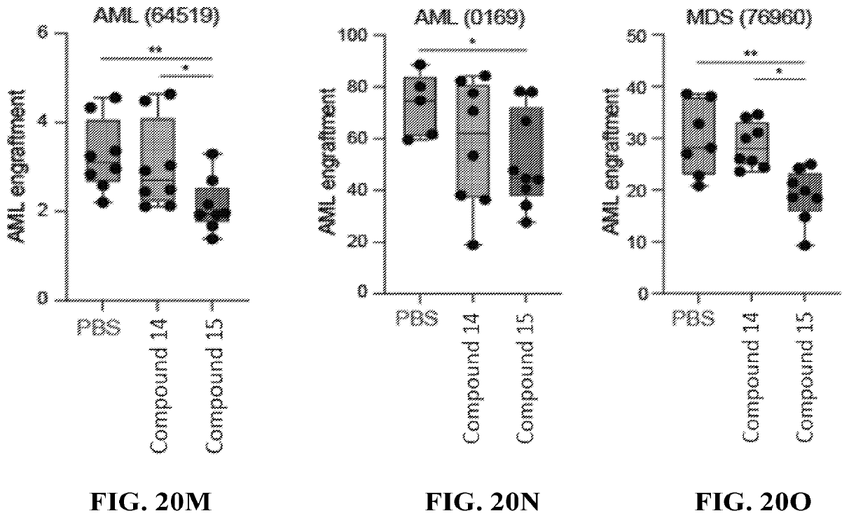

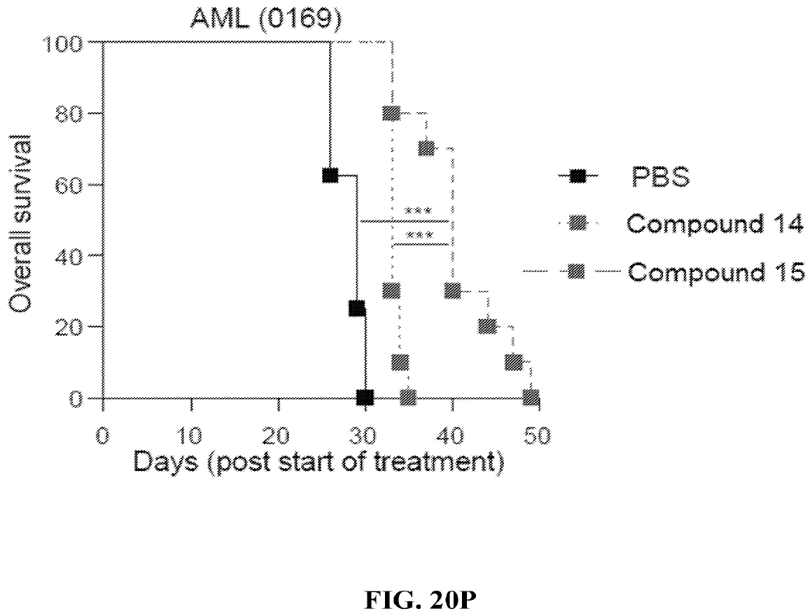

The complimentary and compensatory nature of IRAK1 and IRAK4 activation in AML cells necessitates concomitant inhibition of both targets to maximize therapeutic efficacy. Therefore, a small-molecule inhibitor was desired that simultaneously targeted the IRAK1 and IRAK4 kinase functions. IRAK1 and IRAK4 retain a high degree of structural homology, enabling the generation of dual selective compounds. NCGC1481, derived from a 3-(pyridine-2-yl)imidazole[1,2-a]pyridines backbone with inhibitory activity against IRAK1 and IRAK4, was utilized as a chemical starting point for further optimization of a dual IRAK1/4 inhibitor. Structure activity relationship exploration of this core scaffold yielded a series of small molecules that potently targeted IRAK1 and IRAK4. In functional biochemical assays, Compound 15 exhibited high affinity binding (dissociation constant, Ka) and potent inhibition (inhibitory constant, IC₅₀) of both IRAK1 (K_{d}=2.2 nM; IC₅₀=32 nM) and IRAK4 (K_{d}=0.2 nM; IC₅₀=0.9 nM) (FIG. 20A). A structurally similar derivative was also identified (Compound 14) with selectivity for IRAK4 (K_{d}=5 nM; IC₅₀=5 nM), but not IRAK1 (K_{d}=150 nM; IC₅₀>500 nM) (FIG. 20A). Whereas Compound 14 and Compound 15 were equipotent IRAK4 inhibitors, their differential potency against IRAK1 allowed for the evaluation of the additional therapeutic benefit of targeting IRAK1. It was first confirmed that the small molecules inhibit canonical IRAK1/4-mediated signaling in AML cells upon TLR stimulation. TLR2-mediated activation of NF-kB was selected as a readout as a readout as it was previously found that combined deletion of IRAK1 and IRAK4 is needed to completely inhibit NF-kB activation (FIG. 10E). The dual IRAK1/4 inhibitor Compound 15 (IC₅₀=4.5 nM) was significantly more effective at suppressing TLR2-mediated activation of NF-kB in THP1 cells as compared to the IRAK4 inhibitor Compound 14 (IC₅₀=155 nM) (FIG. 20B). Moreover, Compound 15 completely suppressed TLR2-mediated activation of NF-kB at 100 nM while Compound 14 achieved only ˜80% suppression of the pathway at 5 μM (FIG. 20B). As expected, Compound 15 also inhibited auto-phosphorylation of IRAK1 in THP1 and MDSL cells, while Compound 14 did not affect phosphorylation of IRAK1 (FIG. 20C). These findings indicate that Compound 15 is more effective at suppressing canonical signaling via TLR2 due to combined targeting of IRAK1 and IRAK4 as compared to the selective IRAK4 inhibitor Compound 14.

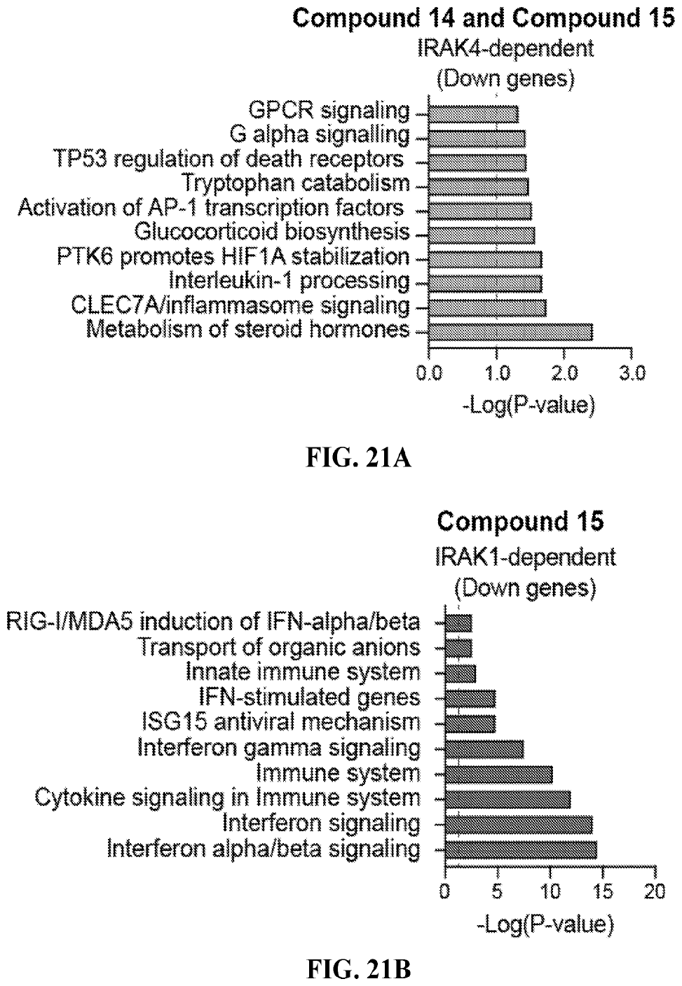

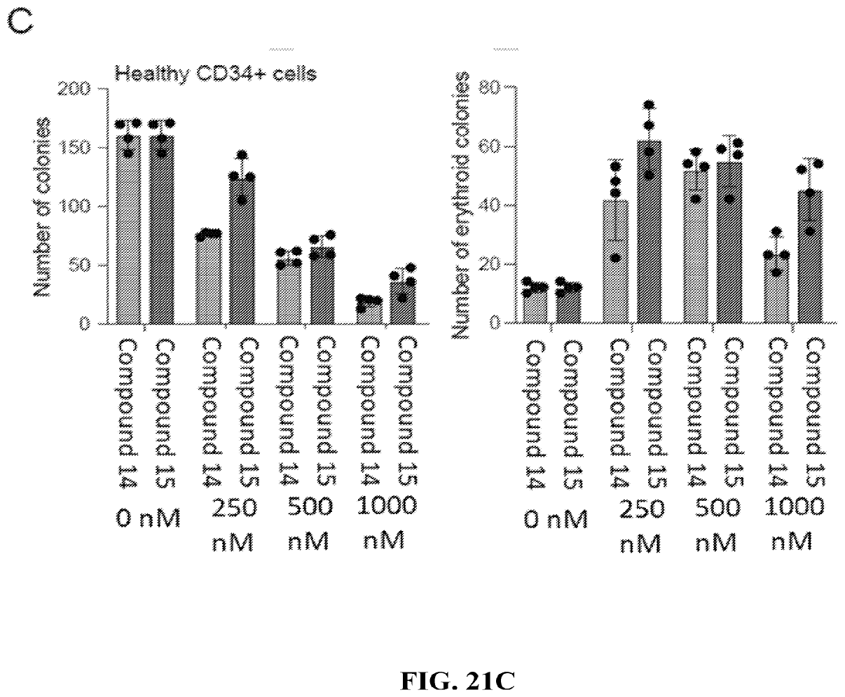

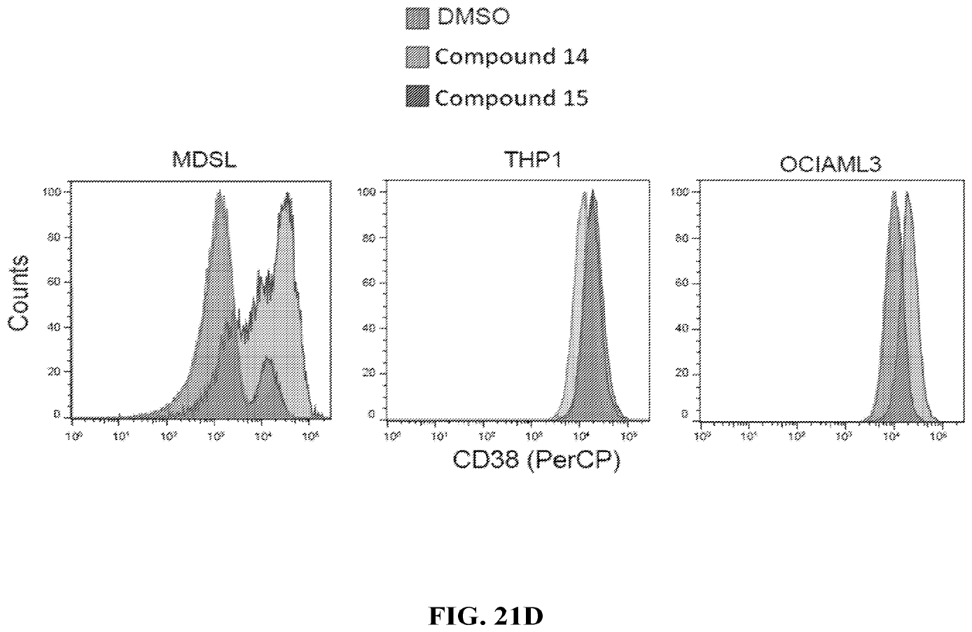

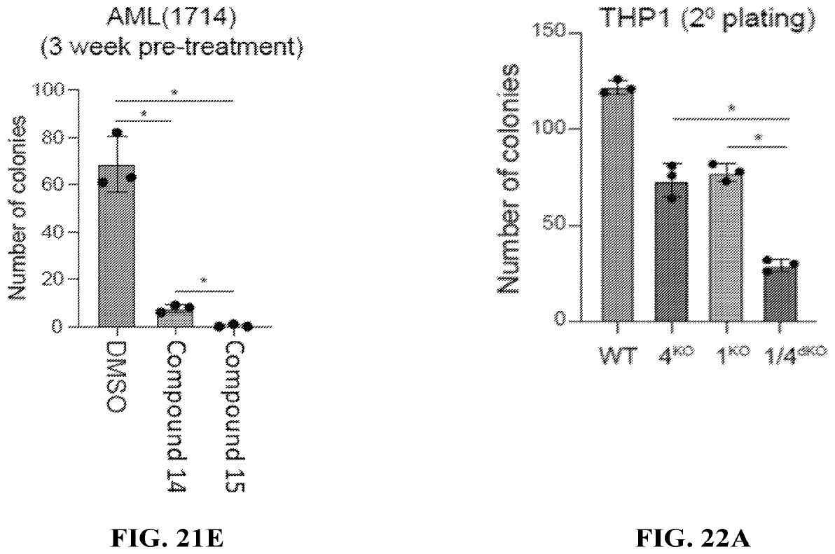

The gene expression changes were next compared in AML cells treated with the dual IRAK1/4 inhibitor Compound 15 and the IRAK4 inhibitor Compound 14. As expected, there were a set of common genes downregulated (“IRAK4-dependent genes”) following treatment with both inhibitors relative to vehicle-treated cells (FIG. 20D). These genes were enriched in MAPK/AP1, ATF4, IGFBP, and EGFR signaling (FIG. 21A). However, treatment with the dual IRAK1/4 inhibitor Compound 15 also resulted in downregulated genes that remained expressed in cells treated with Compound 14 (“IRAK1-dependent genes”) (FIG. 20D). Consistent with the observation that IRAK1 mediates JAK/STAT/interferon signaling (FIGS. 14A-14G, 16A-16H, and 18A-18G), the genes selectively suppressed by the dual IRAK1/4 inhibitor Compound 15, but not the IRAK4 inhibitor Compound 14, are highly enriched in interferon-related pathways (FIG. 21B). The effectiveness of the IRAK1/4 and IRAK4 inhibitors at suppressing the core “IRAK1/4 AML signature” was also examined (FIG. 181B). The dual IRAK1/4 inhibitor Compound 15 suppressed most genes (˜65%) associated with the “IRAK1/4 AML signature,” while the IRAK4 inhibitor Compound 14 had an unremarkable effect on these genes (FIG. 20D). As such, targeting the kinase function of IRAK1 and IRAK4 results in suppression of gene expression programs that are insufficiently inhibited upon targeting IRAK4 alone.

Lastly, it was tested whether dual IRAK1/4 inhibition is more effective than selective IRAK4 inhibition at suppressing LSPCs by evaluating the activity of Compound 14 and Compound 15 on a panel of MDS/AML cell lines and patient-derived samples (FIG. 20E). The IRAK4 inhibitor Compound 14 resulted in an approximately 75% reduction in leukemia progenitor cell colony formation of all MDS/AML cell lines (60-75%) and patient-derived AML samples evaluated (80-85%) (FIG. 20E). The variation in efficacy of the IRAK4 inhibitors Compound 14, CA-4948, and PF-06650833 at suppressing MDS/AML LSPCs is likely due to the unique physical properties and polypharmacology of each chemical structure. As expected, Compound 15 was more effective at inhibiting leukemia progenitor cell colony formation of the MDS/AML cell lines and patient-derived AML samples (FIG. 20E). At these concentrations, Compound 15 treatment of healthy donor CD34+ cells only modesty inhibited myeloid cell colonies yet increased erythroid colony formation (FIG. 21C). In all samples (except for TIP1 cells), Compound 15 resulted in complete suppression of leukemia progenitor cell colony formation (FIG. 20E). Compound 15 was also more effective than Compound 14 at mediating apoptosis of the AML cell lines and patient-derived samples (FIG. 20F). Wright-Giemsa staining was performed on MDS/AML cell lines and patient-derived samples treated Compound 14 and Compound 15 for 12 days. Dual inhibition of IRAK1/4 with Compound 15 coincided with heterogeneous morphologies and size and increased cytoplasmic to nuclear ratios, suggestive of aberrant differentiation (FIG. 20G). These morphological changes were more prominent in leukemic cells treated with Compound 15 relative to Compound 14. Consistent with the morphological changes, Compound 15 treatment resulted in a greater expression of CD38, a glycoprotein expressed on mature immune cells, as compared to Compound 14 or vehicle (FIG. 21D).

### A Dual IRAK1/4 Inhibitor Suppresses MDS/AML in Xenografted Mice

Impaired cellular differentiation is a hallmark of MDS and AML and treatment approaches that promote differentiation are curative in certain subtypes of AML. Therefore, to confirm that the differentiated state of the leukemic cells upon IRAK1/4 inhibition correlates with suppression of LSPCs in vivo, first the leukemic potential of patient-derived AML cells that were exposed to either the dual IRAK1/4 or selective IRAK4 inhibitor for 21 days in vitro were evaluated (FIG. 20I). Exposure of patient-derived AML cells to Compound 15 corresponded with significantly diminished colony formation in vitro (FIG. 21E) and complete loss of leukemic engraftment and AML development in vivo (FIGS. 20J and 20K). In contrast, targeting IRAK4 alone with Compound 14 only partially suppressed LSPCs in vitro and in vivo. These findings suggest that IRAK1/4 is important for preserving the leukemia-propagating cells, and that targeting IRAK1/4 can induce differentiation and suppress LSPC function and AML development.

To evaluate the therapeutic potential of targeting IRAK1/4 in vivo, xenograft studies were also performed using patient-derived AML and MDS samples. Immunocompromised mice were engrafted with leukemic cells from patients with AML or MDS for two weeks and then treated with vehicle, Compound 14, or Compound 15 daily for up to 48 days (FIG. 20L). Leukemic cell engraftment in the PB was significantly reduced with the dual IRAK1/4 inhibitor for AML(64519)(P=0.007; FIG. 20M), AML(0169)(P=0.04; FIG. 20N), or MDS(76960) (P=0.004; FIG. 20O) as compared to vehicle treated mice. However, the IRAK4 inhibitor Compound 14 did not significantly suppress leukemic cell engraftment during the same time (FIGS. 20M-20O). Collectively, these findings suggest that dual IRAK1 and IRAK4 inhibition is required for maximal suppression of signaling and LSPC function (FIG. 20H). To determine whether the reduced leukemic cell engraftment impacts overall survival, mice engrafted with AML(0169) were monitored. Mice treated with the vehicle or Compound 14 achieved a median survival of 29 and 33 days, respectively (FIG. 20P). In contrast, mice treated with Compound 15 survived significantly longer (median of 40 days) (FIG. 20P). Although not wishing to be limited by theory, these findings suggest that dual IRAK1 and IRAK4 inhibition more effectively suppresses AML as compared to targeting IRAK4 alone.

### DISCUSSION

Cell-intrinsic dysregulation of innate immune and inflammatory-related pathways as well as systemic inflammation have been implicated in myeloid malignancies, including MDS and AML. Specifically, LSPCs exhibit extensive dysregulation of immune and inflammatory pathways, many of which converge on IRAK4 signaling. As such, IRAK1 and IRAK4 have been both independently studied in the context of MDS and AML biology; however, their interdependent and complimentary functions have not been sufficiently explored. Based on the central dogma that targeting IRAK4 is sufficient to suppress IRAK1-dependent signaling, several IRAK4 inhibitors and PROTACs are currently being investigated as therapeutic agents for inflammatory diseases and hematologic malignancies. Initial data from clinical trials evaluating IRAK4 inhibitors in HR-MDS and AML revealed encouraging responses in patients with spliceosome mutations that are known to induce hypermorphic IRAK4 isoforms. A subset of patients without spliceosome mutations also exhibited responses to IRAK4 inhibitors, suggesting that IRAK4 signaling is operational in other subtypes of HR-MDS and AML. Based on the initial clinical data as well as pre-clinical studies, IRAK4 therapies are likely inadequate as monotherapies for hematologic malignancies. Furthermore, despite that signaling redundancy is an evolutionarily conserved feature of the mammalian immune system, insufficient attention has been given to the possibility that the IRAK4 paralog, IRAK1, may functionally compensate and/or compliment IRAK4 deficiency in leukemic cells. Such models of functional compensation or complementation by proximal effectors in signaling pathways are common mechanisms underlying therapy resistance and limited therapeutic efficacy in cancer. Treatment of RAS-mutant cancers with MEK inhibitors, for instance, results in upregulation of other mediators of the MAPK pathway to restore signaling. Similarly, P53 can be inactivated by interaction with either MDM2 or its paralog MDMX; while therapeutic efforts have focused on inhibiting the interaction with MDM2, a recently developed MDM2/MDMX dual-inhibitor achieves superior efficacy in suppressing LSPC function in preclinical AML models. Herein, it was demonstrated that deficiency or inhibition of IRAK4 paradoxically elicits inflammatory signaling pathways, including JAK/STAT/interferon signaling, via upregulation and/or activation of IRAK1 (FIG. 20H). Leveraging genetic and pharmaceutical interventions, it was found that this phenomenon translates to a robust LSPC defect when simultaneously targeting IRAK1 and IRAK4.

Interrogating the physiologic underpinning for the requirement for IRAK1 and IRAK4 in AML, MyD88-independent IRAK1/4 signaling functions were also uncovered. Though IRAK1 and IRAK4 are subject of broad study as central effectors of innate immune responses and recruitment of adaptive immunity, mechanistic dissection of IRAK1/4 functions in myeloid malignancies are lacking. Therapeutic efforts to target IRAK1/4 signaling for these diseases have conveniently adopted paradigms formed in studies of innate immunology that are based exclusively on models of acute receptor stimulation, wherein the adaptor MyD88 nucleates a complex with IRAK4 to activate IRAK1 and initiate signaling through the NF-kB and MAPK pathways. Consequently, IRAK4, as the proximal effector of MyD88-dependent signaling, has been the exclusive focus of therapeutic interventions without consideration for the possibility that IRAK1 and IRAK4 may execute distinct and/or compensatory functions in malignant cells. Here, it was shown that MyD88 is indeed dispensable for MDS/AML LSPC function, while MyD88-deficient LSPCs remain dependent on IRAK1/4 signaling. Through transcriptional and chromatin studies, it was found that MyD88-independent IRAK1/4 signaling is required to preserve the undifferentiated state of LSPCs and that a MyD88-independent “IRAK1/4 signature” is present in a subset of MDS and AIL patients. Proximity labelling to characterize the unique and overlapping interactomes of IRAK1 and IRAK4 did identify components of NF-kB and MAPK signaling as interactors of IRAK1 and IRAK4, however, the majority of proximal proteins mapped to MyD88-independent pathways. For instance, high-confidence interactions were identified between IRAK4 and core constituents of the PRC2 complex, an epigenetic regulator that is requisite for the maintenance of LSPCs. The interaction with the PRC2 complex is consistent with the differentiated phenotype observed upon deletion or inhibition of IRAK4 in AML and corresponding alterations in chromatin accessibility at PRC2-regulated genomic loci. These findings are further corroborated by the enrichment of BCOR and SRSF2 mutations in AML patients with the “IRAK1/4 signature.” BCOR is a component of the PRC1 complex, which is recruited by PRC2 to genomic loci, while SRSF2 mutations cause mis-splicing of the PRC2 component EZH2 and are mutually exclusive with EZH2 mutations in some hematologic malignancies. Collectively, these data argue that the PRC2 complex is a disease-relevant substrate of IRAK4 signaling in MDS/AML.

The IRAK4 interactome also included mediators of AKT signaling and core machinery of receptor tyrosine kinase signaling (GRB2, ERK1, ERK2, RAC1), indicating a role for IRAK4 in growth factor signal transduction. Thus, IRAK4 is far more dynamic than is ascribed by models of acute TLR stimulation. It was found that high-confidence IRAK1 interactions mapped to mediators of JAK/STAT/interferon signaling, which could account for the induction of a JAK/STAT/interferon signaling observed upon genetic or pharmaceutical deficiency of IRAK4. While excessive interferon signaling can suppress normal and malignant hematopoiesis, recent reports indicate a requirement for tonic, sterile interferon signaling in LSPC maintenance. Notably, activating JAK2 mutations are also significantly enriched in AML patients with the “IRAK1/4 signature,” further suggesting that IRAK1 coordinates JAK/STAT/interferon signaling. Collectively, these studies resolve novel MyD88-independent signaling mechanisms for IRAK1 and IRAK4 and present a model wherein (1) IRAK4 regulates epigenetic machinery and signaling pathways that mediate a differentiation block in leukemic cells, and (2) targeting of IRAK4 upregulates IRAK1 to activate complimentary signaling pathways, including the JAK/STAT/interferon axis (FIG. 20H).

As this model presents a rational basis for the design of dual IRAK1/4 targeted therapy, a set of structurally similar small molecule inhibitors were developed with selectivity for IRAK4 (Compound 14) or dual-selectivity for IRAK1 and IRAK4 (Compound 15). In line with genetic models, the dual IRAK1/4 inhibitor induced differentiation and suppressed the function of LSPCs more effectively than the IRAK4-selective inhibitor. Furthermore, it was found that genes that are downregulated upon treatment with the dual IRAK1/4 inhibitor, but not the IRAK4-selective inhibitor, are significantly enriched for JAK/STAT/interferon signatures. This finding confirms that activation of JAK/STAT/interferon signaling by IRAK1 can be suppressed by pharmacological inhibition and suggests that this may underly a therapeutic advantage for dual IRAK1/4 therapy.

Although AML was relied on as a model system, it is anticipated that the MyD88-independent complimentary functions of IRAK1 and IRAK4 can be extended to other pathologies and perhaps normal immunobiology. Both IRAK1 and IRAK4 are implicated as effectors of pathogenesis, stemness, and chemoresistance in numerous solid tissue tumors. Thus, IRAK1/4-targeted therapies may gain traction for malignancies of diverse tissue types, lending greater importance to the optimization of treatment strategies. Whether the roles of IRAK1 and IRAK4 in solid tissue tumors are attributable to MyD88-independent signaling, and whether IRAK1 and IRAK4 complement each other in these contexts remains unknown. Addressing these questions will be critical in determining if a therapeutic mandate for targeting of IRAK1 and IRAK4 is broadly applicable. Collectively, it was demonstrated that IRAK1 and IRAK4 mediate compensatory and complementary MyD88-independent functions that underly a need for dual IRAK1/4 inhibitors. The findings also reveal that IRAK1 and IRAK4 signaling is dynamic with wide-ranging implications for treating malignancies and inflammatory disease.

### REFERENCES

1. Cazzola, M. Myelodysplastic Syndromes. N Engl J Med 383, 1358-1374 (2020).
2. Kantarjian, H., et al. Acute myeloid leukemia: current progress and future directions.

### Blood Cancer J 11, 41 (2021).

3. Pandolfi, A., Barreyro, L. & Steidl, U. Concise review: preleukemic stem cells: molecular biology and clinical implications of the precursors to leukemia stem cells. Stem Cells Transl Med 2, 143-150 (2013).
4. Shastri, A., Will, B., Steidl, U. & Verma, A. Stem and progenitor cell alterations in myelodysplastic syndromes. Blood 129, 1586-1594 (2017).
5. Stauber, J., Greally, J. M. & Steidl, U. Preleukemic and leukemic evolution at the stem cell level. Blood 137, 1013-1018 (2021).
6. Daver, N., et al. New directions for emerging therapies in acute myeloid leukemia: the next chapter. Blood Cancer J 10, 107 (2020).
7. Krivtsov, A. V., et al. A Menin-MLL Inhibitor Induces Specific Chromatin Changes and Eradicates Disease in Models of MLL-Rearranged Leukemia. Cancer Cell 36, 660-673 e611 (2019).
8. Lane, S. W. & Gilliland, D. G. Leukemia stem cells. Semin Cancer Biol 20, 71-76 (2010).
9. Barreyro, L., et al. Blocking UBE2N abrogates oncogenic immune signaling in acute myeloid leukemia. Sci Transl Med 14, eabb7695 (2022).
10. Trowbridge, J. J. & Starczynowski, D. T. Innate immune pathways and inflammation in hematopoietic aging, clonal hematopoiesis, and MDS. J Exp Med 218(2021).
11. Sallman, D. A. & List, A. The central role of inflammatory signaling in the pathogenesis of myelodysplastic syndromes. Blood 133, 1039-1048 (2019).
12. Varney, M. E., et al. Deconstructing innate immune signaling in myelodysplastic syndromes. Exp Hematol 43, 587-598 (2015).
13. Starczynowski, D. T. & Karsan, A. Innate immune signaling in the myelodysplastic syndromes. Hematol Oncol Clin North Am 24, 343-359 (2010).
14. Barreyro, L., Chlon, T. M. & Starczynowski, D. T. Chronic immune response dysregulation in MDS pathogenesis. Blood 132, 1553-1560 (2018).
15. Monlish, D. A., Bhatt, S. T. & Schuettpelz, L. G. The Role of Toll-Like Receptors in Hematopoietic Malignancies. Front Immunol 7, 390 (2016).
16. Gañán-Gómez, I., et al. Deregulation of innate immune and inflammatory signaling in myelodysplastic syndromes. Leukemia 29, 1458 (2015).
17. Bennett, J. & Starczynowski, D. T. IRAK1 and IRAK4 as emerging therapeutic targets in hematologic malignancies. Curr Opin Hematol 29, 8-19 (2022).
18. Fitzgerald, K. A. & Kagan, J. C. Toll-like Receptors and the Control of Immunity. Cell 180, 1044-1066 (2020).
19. Yu, X., et al. MYD88 L265P Mutation in Lymphoid Malignancies. Cancer research 78, 2457-2462 (2018).
20. Winkler, A., et al. The Interleukin-1 Receptor-Associated Kinase 4 Inhibitor PF-06650833 Blocks Inflammation in Preclinical Models of Rheumatic Disease and in Humans Enrolled in a Randomized Clinical Trial. Arthritis Rheumatol 73, 2206-2218 (2021).
21. Gummadi, V. R., et al. Discovery of CA-4948, an Orally Bioavailable IRAK4 Inhibitor for Treatment of Hematologic Malignancies. ACS Med Chem Lett 11, 2374-2381 (2020).
22. Stevens, D., et al. Phase 1 Study of KT-413, a Targeted Protein Degrader of IRAK4 and IMiD Substrates, in Adult Patients with Relapsed or Refractory B-Cell Non-Hodgkin Lymphoma. in ASCO (2022).
23. Eric Kuhn, et al. Discovery and targeted mass spectrometry-based proteomics: Enabling technologies advancing IRAK4 protein degrader along the drug discovery pipeline into the clinic. in ASMS (2022).
24. Garcia-Manero, G., et al. Takeaim Leukemia-A Phase 1/2a Study Of The IRAK4 Inhibitor Emavusertib (Ca-4948) as Monotherapy or in Combination with Azacitidine or Venetoclax in Relapsed/Refractory AML or MDS. In EHA (ed. HemaSphere) (2022).
25. Choudhary, G. S., et al. Activation of targetable inflammatory immune signaling is seen in myelodysplastic syndromes with SF3B1 mutations. Elife 11(2022).
26. Smith, M. A., et al. U2AF1 mutations induce oncogenic IRAK4 isoforms and activate innate immune pathways in myeloid malignancies. Nat Cell Biol 21, 640-650 (2019).
27. Lam, S. S., et al. Directed evolution of APEX2 for electron microscopy and proximity labeling. Nat Methods 12, 51-54 (2015).
28. Zhou, Y., et al. Metascape provides a biologist-oriented resource for the analysis of systems-level datasets. Nat Commun 10, 1523 (2019).
29. Pawlus, MR., Wang, L. & Hu, C. J. STAT3 and HIF1alpha cooperatively activate HIF1 target genes in MDA-MB-231 and RCC4 cells. Oncogene 33, 1670-1679 (2014).
30. Szklarczyk, D., et al. The STRING database in 2021: customizable protein-protein networks, and functional characterization of user-uploaded gene/measurement sets. Nucleic Acids Res 49, D605-D612 (2021).
31. Iwama, A. Polycomb repressive complexes in hematological malignancies. Blood 130, 23-29 (2017).
32. Wingelhofer, B., et al. Implications of STAT3 and STAT5 signaling on gene regulation and chromatin remodeling in hematopoietic cancer. Leukemia 32, 1713-1726 (2018).
33. Chi, P., Allis, C. D. & Wang, G. G. Covalent histone modifications--miswritten, misinterpreted and mis-erased in human cancers. Nat Rev Cancer 10, 457-469 (2010).
34. Giotopoulos, G., et al. The epigenetic regulators CBP and p300 facilitate leukemogenesis and represent therapeutic targets in acute myeloid leukemia. Oncogene 35, 279-289 (2016).
35. Feng, Y., Li, L., Du, Y., Peng, X. & Chen, F. E2F4 functions as a tumour suppressor in acute myeloid leukaemia via inhibition of the MAPK signalling pathway by binding to EZH2. J Cell Mol Med 24, 2157-2168 (2020).
36. Tyner, J. W., et al. Functional genomic landscape of acute myeloid leukaemia. Nature 562, 526-531 (2018).
37. Melgar, K., et al. Overcoming adaptive therapy resistance in AIL by targeting immune response pathways. Sci Transl Med 11(2019).
38. Jones, L. M., et al. Targeting AML-associated FLT3 mutations with a type I kinase inhibitor. J Clin Invest 130, 2017-2023 (2020).
39. Rhyasen, G. W., Bolanos, L. & Starczynowski, D. T. Differential IRAK signaling in hematologic malignancies. Exp Hematol 41, 1005-1007 (2013).
40. Rhyasen, G. W., et al. Targeting IRAK1 as a therapeutic approach for myelodysplastic syndrome. Cancer Cell 24, 90-104 (2013).
41. Hosseini, M. M., et al. Inhibition of interleukin-1 receptor-associated kinase-1 is a therapeutic strategy for acute myeloid leukemia subtypes. Leukemia 32, 2374-2387 (2018).
42. Stoner, S. A., et al. Hippo kinase loss contributes to del(20q) hematologic malignancies through chronic innate immune activation. Blood 134, 1730-1744 (2019).
43. Liang, K., et al. Therapeutic Targeting of MLL Degradation Pathways in MLL-Rearranged Leukemia. Cell 168, 59-72 e13 (2017).
44. Nish, S. & Medzhitov, R. Host defense pathways: role of redundancy and compensation in infectious disease phenotypes. Immunity 34, 629-636 (2011).
45. Kun, E., Tsang, Y. T. M., Ng, C. W., Gershenson, D. M. & Wong, K. K. MEK inhibitor resistance mechanisms and recent developments in combination trials. Cancer Treat Rev 92, 102137 (2021).
46. Carvajal, L. A., et al. Dual inhibition of MDMX and MDM2 as a therapeutic strategy in leukemia. Sci Transl Med 10(2018).
47. Kawasaki, T. & Kawai, T. Toll-like receptor signaling pathways. Front Immunol 5, 461 (2014).
48. Aujla, A., Linder, K., Iragavarapu, C., Karass, M. & Liu, D. SRSF2 mutations in myelodysplasia/myeloproliferative neoplasms. Biomark Res 6, 29 (2018).
49. Kim, E., et al. SRSF2 Mutations Contribute to Myelodysplasia by Mutant-Specific Effects on Exon Recognition. Cancer Cell 27, 617-630 (2015).
50. Musella, M., et al. Type I IFNs promote cancer cell stemness by triggering the epigenetic regulator KDM1B. Nat Immunol 23, 1379-1392 (2022).
51. Zhang, D., et al. Tumor-Stroma ILlbeta-IRAK4 Feedforward Circuitry Drives Tumor Fibrosis, Chemoresistance, and Poor Prognosis in Pancreatic Cancer. Cancer research 78, 1700-1712 (2018).
52. Li, Q., et al. IRAK4 mediates colitis-induced tumorigenesis and chemoresistance in colorectal cancer. JCI Insight 4(2019).
53. Liu, L., et al. Targeting the IRAK1-S100A9 Axis Overcomes Resistance to Paclitaxel in Nasopharyngeal Carcinoma. Cancer research 81, 1413-1425 (2021).
54. Adams, A. K., et al. IRAK1 is a novel DEK transcriptional target and is essential for head and neck cancer cell survival. Oncotarget 6, 43395-43407 (2015).
55. Wee, Z. N., et al. IRAK1 is a therapeutic target that drives breast cancer metastasis and resistance to paclitaxel. Nat Commun 6, 8746 (2015).
56. Cheng, B. Y., et al. IRAK1 Augments Cancer Stemness and Drug Resistance via the AP-1/AKR1B10 Signaling Cascade in Hepatocellular Carcinoma. Cancer research 78, 2332-2342 (2018).
57. Hu, Q., et al. miR-146a promotes cervical cancer cell viability via targeting IRAK1 and TRAF6. Oncol Rep 39, 3015-3024 (2018).
58. Wickham, H. Ggplot2: Elegant Graphics for Data Analysis, (2009).

### Example 4

All AML patients become resistant/refractory to existing therapies. The leukemic cells of these patients exhibit dysregulation of innate immune signaling pathways upon diagnosis, wherein these pathways become further activated in drug resistance. The IRAK1/IRAK4 kinase complex is part of a critical signaling node that becomes activated in these dysregulated pathways. Multiple genetic alterations in MDS/AML result in activation of IRAK1/4 pathways. IRAK1/4 regulates genes involved in cancer cell survival, self-renewal, and stress response via the NF-kB, MAPK, and potentially, the inflammasome pathways. IRAK1/4 is involved in cancer cell dependencies and adaptive resistance mechanisms. IRAK4 exists as 2 major isoforms: IRAK4-L (“hyperactive”) and IRAK4-S(“less active”). IRAK1 is overexpressed and activated in myeloid malignancies. The IRAK1/4 inhibitors of the present disclosure work by targeting key cancer survival pathways and thereby increase overall survival (OS) of patients with hematopoietic and solid tumors, prolong remission rates, and/or increase the success of a stem cell transplant (HSCT). The IRAK 1/4 inhibitors of the present disclosure also synergize with Venetoclax and are thus positioned to treat cancers such as AML with a higher potency than other targeted therapies that are currently available.

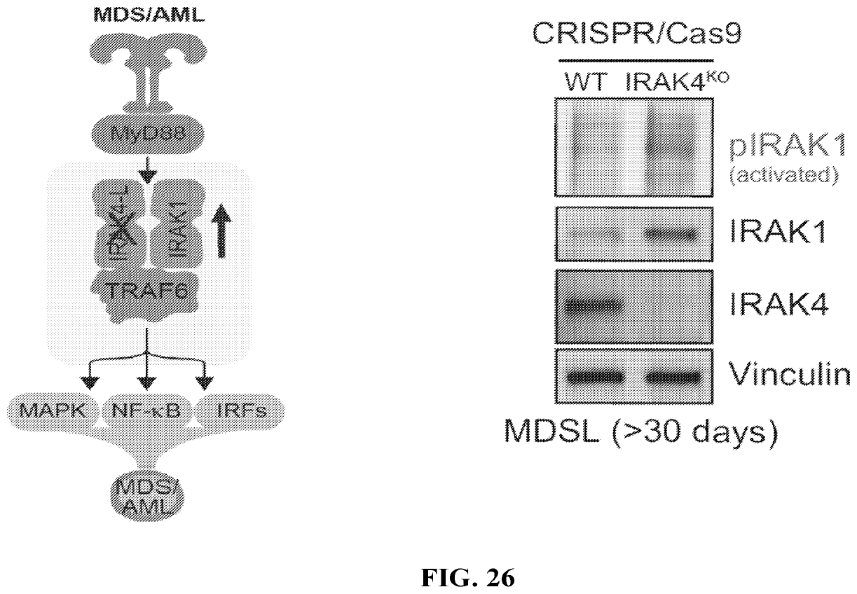

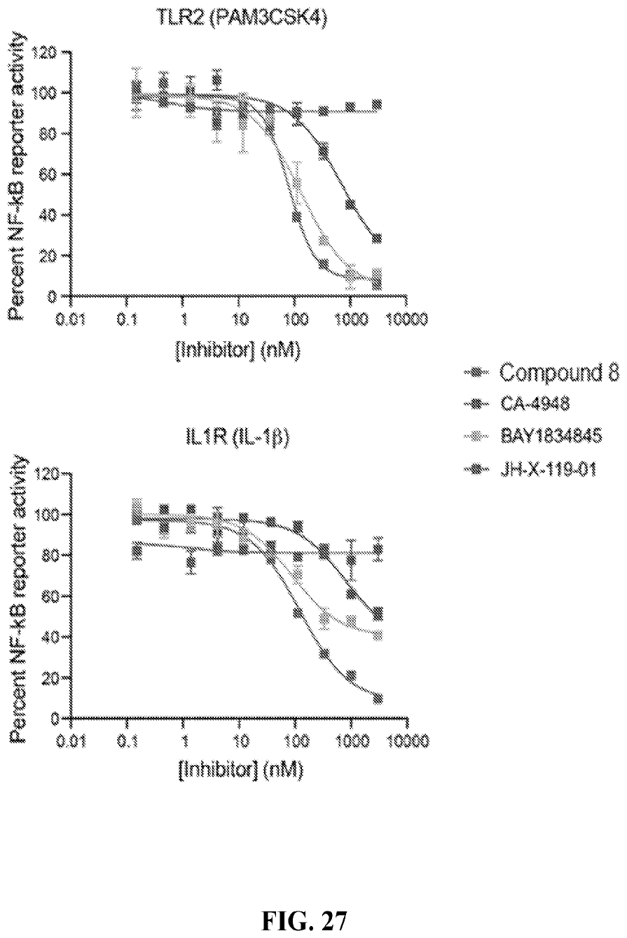

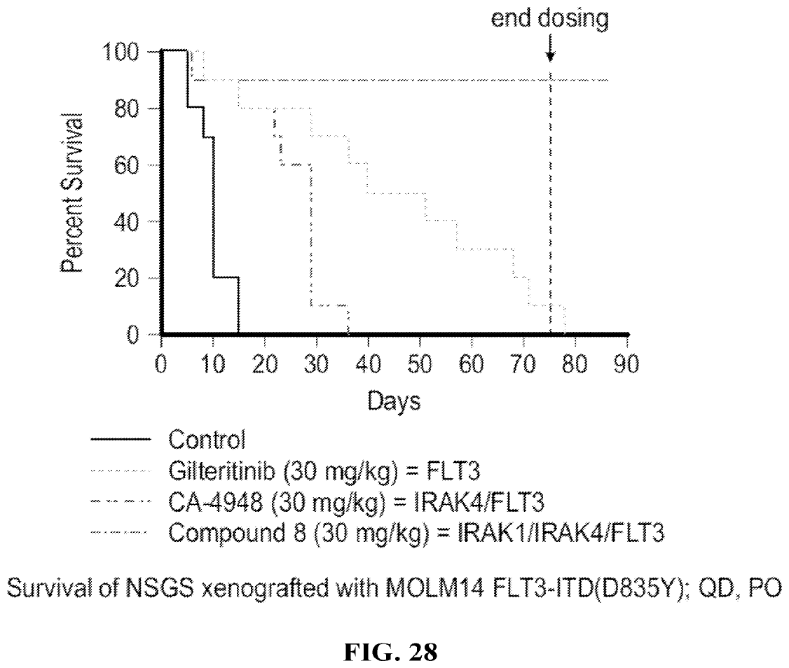

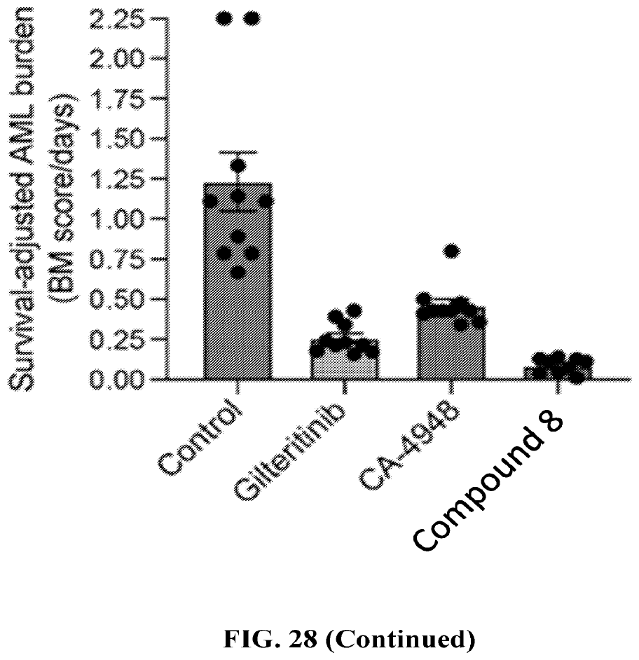

Dual IRAK1/4 inhibition was found to be required to drive maximal efficacy (FIGS. 20E-20F). Specifically, it was discovered that: 1) IRAK1 is activated in response to IRAK4 inhibition or degradation, 2) the complete inhibition of NF-kB-derived signaling through multiple receptors requires inhibition of both IRAK1 and IRAK4 (FIG. 28), and 3) genetic ablation of both IRAK1 and IRAK4 is more effective in promoting survival in AML xenograft models than is deletion of either kinase alone (FIGS. 11H-11I). IRAK1 and IRAK4 mediate distinct and overlapping signaling pathways in AML. In leukemia, IRAK1 compensates for IRAK4 inhibition and therefore IRAK4 inhibition is insufficient for full efficacy. Targeting IRAK4 was found to result in compensation by IRAK1 wherein IRAK1 is upregulated and activated upon IRAK4 deletion (FIG. 26). Similar results were also obtained with IRAK4 inhibitors or degraders. FIG. 27 depicts that complete antagonism of signaling through both receptor pathways is only seen with an IRAK1/4 inhibitor of the present disclosure while the irreversible IRAK1 inhibitor JH-X-119-01 does not inhibit signaling through either receptor and the IRAK4 selective inhibitors do not completely inhibit signaling through IL1R. Furthermore, an IRAK1/4 inhibitor of the present disclosure was found to provide enhanced survival in xenograft mice compared to emavusertib (CA-4948) and gilteritinib (FIG. 28).

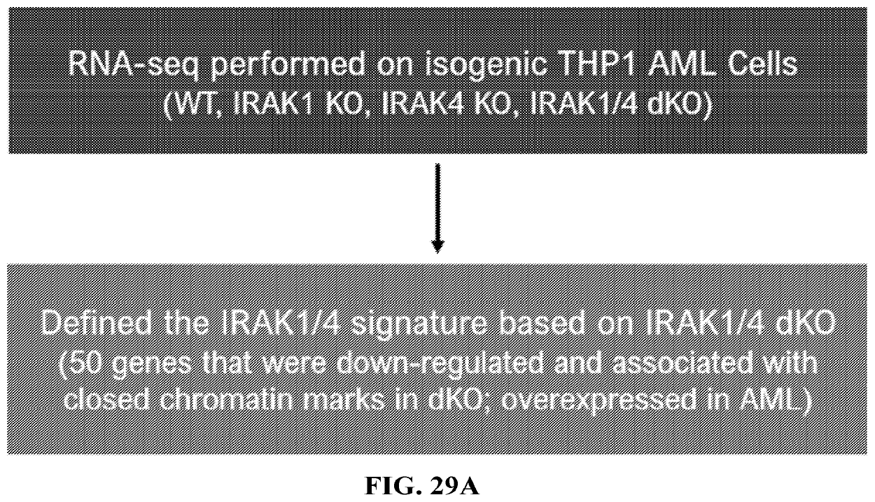

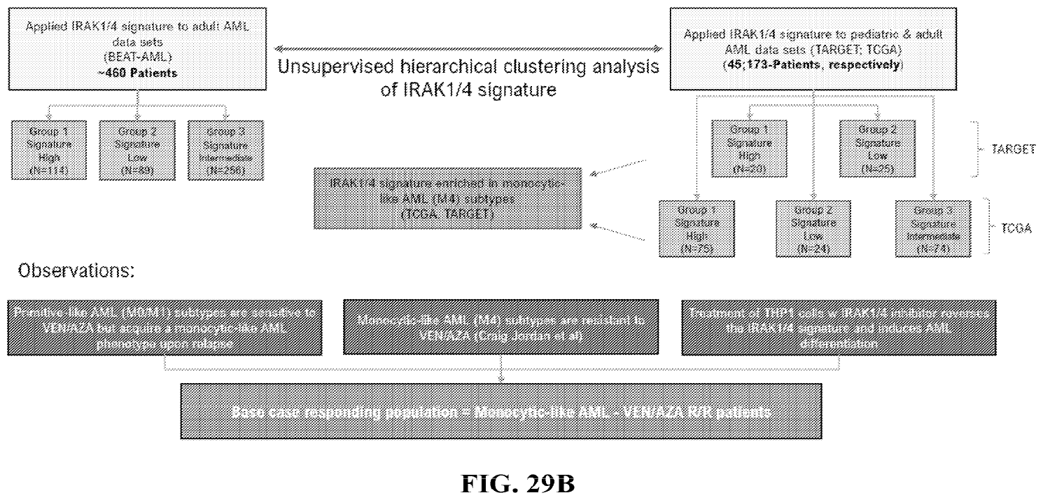

The in vitro and in vivo studies were used to develop an IRAK1/4 signature in patients (FIGS. 29A-29B). Based on the development of the IRAK1/4 signature (see Example 3), the data suggest:
1) the IRAK1/4 signature is present in AML patients throughout the BEAT-AML, TCGA, and TARGET databases indicating that IRAK1/4 signaling is operational in a significant cohort of pediatric and adult AMLs,
2) the highest levels occur in 24.8%, 43.4% of adult and 44.4% of pediatric AML patients,
3) the highest levels of expression are found in the monocytic-like (M4) AML patients (adult and pediatric), and
4) since the monocytic-like AML patients are also those who are resistant to hypomethylating agents/venetoclax (HMA/VEN), the data suggest that the population most likely to respond to an IRAK1/4 inhibitor will be the HMA/VEN resistant population.

While the commercially available IRAK4 inhibitor emavusertib (CA-4948) shows early efficacy in relapsed/refractory (R/R) MDS/AML patients previously treated with HMA/VEN, this efficacy is only seen in a sub-population of HMA/VEN R/R patients (spliceosome-mutant patients) which have been found to have increased expression of activated IRAK4. Therefore, the highest efficacy is seen in MDS patients, where the incidence of spliceosome mutations is higher and lower efficacy is seen in AML (as predicted by the comparative data herein). This further demonstrates the need for dual IRAK1/4 inhibition in AML patients. The dual IRAK1/4 inhibitors disclosed herein have been demonstrated to have superior efficacy vs. IRAK4 inhibitors in multiple orthogonal assays. Furthermore, the IRAK1/4 signature suggests that a clinical effect will be observed in a large proportion of AML/MDS patients treated with an IRAK1/4 inhibitor of the present disclosure, including the HMA/VEN R/R population.

It is noted that terms like “preferably,” “commonly,” and “typically” are not used herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure.

The various methods and techniques described above provide a number of ways to carry out the disclosure. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the disclosure extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. As used in the disclosure or claims, “another” means at least a second or more, unless otherwise specified. As used in the disclosure, the phrases “such as,” “for example,” and “e.g.” mean “for example, but not limited to” in that the list following the term (“such as,” “for example,” or “e.g.”) provides some examples but the list is not necessarily a fully inclusive list. The word “comprising” means that the items following the word “comprising” may include additional unrecited elements or steps; that is, “comprising” does not exclude additional unrecited steps or elements. No language in the specification should be construed as indicating any non-claimed element essential to the practice of the application.

In certain instances, sequences disclosed herein are included in publicly available databases, such as GENBANK© and SWISSPROT. Unless otherwise indicated or apparent the references to such publicly available databases are references to the most recent version of the database as of the filing date of this Application.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter. As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

### Appendix A

TABLE 2
Differentially expressed genes with
CA-4948 vs DMSO in THP1 cells

| Gene | Log(FC) | P Value | Adj P Value |
|---|---|---|---|
| CRABP2 | 3.095 | 5.57E−08 | 5.57E−08 |
| TNXB | 1.806 | 5.79E−10 | 5.79E−10 |
| EFCAB12 | 1.697 | 8.48E−09 | 8.48E−09 |
| FLJ45513 | 1.668 | 2.14E−06 | 2.14E−06 |
| NPIPA3 | 1.643 | 0.0004833 | 0.0004833 |
| COL7A1 | 1.605 | 1.38E−08 | 1.38E−08 |
| WNK4 | 1.507 | 2.94E−09 | 2.94E−09 |
| SMIM11 | 1.485 | 0.03161 | 0.03161 |
| LOC102724409 | 1.429 | 0.003556 | 0.003556 |
| LOC102723975 | 1.408 | 9.25E−06 | 9.25E−06 |
| GNMT | 1.408 | 2.24E−05 | 2.24E−05 |
| ZDHHC11 | 1.406 | 2.62E−06 | 2.62E−06 |
| DND1 | 1.389 | 3.35E−05 | 3.35E−05 |
| SCART1 | 1.38 | 9.59E−05 | 9.59E−05 |
| GUCA1B | 1.348 | 2.21E−05 | 2.21E−05 |
| BZRAP1 | 1.334 | 4.00E−10 | 4.00E−10 |
| CPT1B | 1.318 | 0.007755 | 0.007755 |
| LOC101927910 | 1.281 | 0.0002476 | 0.0002476 |
| ASIC3 | 1.28 | 0.0001091 | 0.0001091 |
| DCST2 | 1.278 | 0.0007274 | 0.0007274 |
| SPTBN5 | 1.272 | 4.22E−09 | 4.22E−09 |
| PRR22 | 1.272 | 2.11E−08 | 2.11E−08 |
| LOC100507424 | 1.26 | 0.0008331 | 0.0008331 |
| CEACAM19 | 1.259 | 5.73E−08 | 5.73E−08 |
| ITIH4 | 1.233 | 2.41E−08 | 2.41E−08 |
| LOC100129083 | 1.194 | 1.63E−06 | 1.63E−06 |
| LINC01133 | 1.19 | 2.63E−09 | 2.63E−09 |
| C1R | 1.188 | 0.001884 | 0.001884 |
| UCN | 1.177 | 2.18E−06 | 2.18E−06 |
| SPATC1 | 1.175 | 0.0001674 | 0.0001674 |
| FCRLB | 1.162 | 0.0004631 | 0.0004631 |
| CMTM1 | 1.158 | 6.22E−07 | 6.22E−07 |
| PIP5KL1 | 1.154 | 2.27E−08 | 2.27E−08 |
| PRSS57 | 1.151 | 1.71E−05 | 1.71E−05 |
| FBXO39 | 1.15 | 0.0003452 | 0.0003452 |
| SLC23A3 | 1.148 | 0.0002647 | 0.0002647 |
| AOC2 | 1.143 | 0.0003823 | 0.0003823 |
| SLC25A34 | 1.141 | 1.53E−05 | 1.53E−05 |
| TMEM249 | 1.14 | 8.22E−07 | 8.22E−07 |
| TREML1 | 1.14 | 1.20E−06 | 1.20E−06 |
| ZNF205-AS1 | 1.14 | 0.001593 | 0.001593 |
| MIR3187 | 1.121 | 0.004933 | 0.004933 |
| LOC100506548 | 1.108 | 2.85E−17 | 2.85E−17 |
| DCAF4L1 | 1.097 | 0.0004582 | 0.0004582 |
| MYLK-AS1 | 1.091 | 0.003903 | 0.003903 |
| DNHD1 | 1.083 | 1.80E−12 | 1.80E−12 |
| CLK1 | 1.08 | 1.80E−16 | 1.80E−16 |
| LHPP | 1.078 | 0.000127 | 0.000127 |
| POTEE | 1.077 | 0.01333 | 0.01333 |
| MIR570 | 1.064 | 0.0003682 | 0.0003682 |
| SNORD30 | 1.062 | 0.001393 | 0.001393 |
| C1orf51 | 1.033 | 7.31E−05 | 7.31E−05 |
| CTRL | 1.027 | 5.78E−05 | 5.78E−05 |
| YJEFN3 | 1.024 | 2.47E−09 | 2.47E−09 |
| ADCY4 | 1.023 | 2.42E−07 | 2.42E−07 |
| LOC100129924 | 1.019 | 0.003331 | 0.003331 |
| POLN | 1.016 | 0.0002137 | 0.0002137 |
| DNASE1L2 | 1.008 | 1.64E−06 | 1.64E−06 |
| IL9R | 1.002 | 0.01654 | 0.01654 |
| TMEM145 | 0.9943 | 0.0003413 | 0.0003413 |
| LOC100128494 | 0.9913 | 0.002477 | 0.002477 |
| LOC102723959 | 0.9881 | 7.52E−05 | 7.52E−05 |
| RUFY4 | 0.9698 | 1.92E−06 | 1.92E−06 |
| CCDC183 | 0.9658 | 2.33E−05 | 2.33E−05 |
| SAP25 | 0.962 | 3.86E−08 | 3.86E−08 |
| HSPA7 | 0.9538 | 0.0001058 | 0.0001058 |
| CNIH2 | 0.9526 | 0.0015 | 0.0015 |
| IGFBP7-AS1 | 0.9516 | 0.003228 | 0.003228 |
| FAM57B | 0.9462 | 7.12E−07 | 7.12E−07 |
| ZP3 | 0.9339 | 4.20E−05 | 4.20E−05 |
| CLK3 | 0.9323 | 4.28E−17 | 4.28E−17 |
| ZSWIM8-AS1 | 0.9316 | 0.0005395 | 0.0005395 |
| ANKRD34A | 0.9288 | 0.0008014 | 0.0008014 |
| CNKSR1 | 0.9226 | 0.003884 | 0.003884 |
| RPS18P9 | 0.9157 | 0.000963 | 0.000963 |
| QRICH2 | 0.9066 | 0.0002348 | 0.0002348 |
| CGN | 0.9021 | 0.004276 | 0.004276 |
| DRP2 | 0.9019 | 0.0002334 | 0.0002334 |
| CCDC109B | 0.8991 | 0.001084 | 0.001084 |
| CILP2 | 0.8989 | 0.002546 | 0.002546 |
| LOC100506100 | 0.8985 | 0.000907 | 0.000907 |
| EGFR | 0.882 | 0.01651 | 0.01651 |
| LOC101928038 | 0.8781 | 0.005726 | 0.005726 |
| TMEM9B-AS1 | 0.8758 | 0.001738 | 0.001738 |
| ARID3C | 0.8751 | 0.008951 | 0.008951 |
| SCXB | 0.8721 | 1.46E−05 | 1.46E−05 |
| C15orf62 | 0.8673 | 0.007947 | 0.007947 |
| VWA7 | 0.8656 | 0.001993 | 0.001993 |
| LOC101928055 | 0.8595 | 0.000212 | 0.000212 |
| STX18-AS1 | 0.8594 | 0.005636 | 0.005636 |
| CHN2 | 0.8531 | 0.000282 | 0.000282 |
| LOC102724545 | 0.8513 | 0.003253 | 0.003253 |
| FAM229A | 0.8452 | 0.002954 | 0.002954 |
| LOC100130705 | 0.845 | 0.003108 | 0.003108 |
| LOC100134868 | 0.8444 | 0.02866 | 0.02866 |
| MROH6 | 0.8417 | 2.51E−10 | 2.51E−10 |
| LOC101929264 | 0.8417 | 0.001338 | 0.001338 |
| PAPPA2 | 0.8397 | 0.003118 | 0.003118 |
| LOC284889 | 0.8366 | 3.31E−05 | 3.31E−05 |
| TEKT4P2 | 0.8324 | 0.01705 | 0.01705 |
| LINC00641 | 0.8241 | 1.47E−09 | 1.47E−09 |
| SLC7A5P2 | 0.8202 | 0.001087 | 0.001087 |
| NPIPA8 | 0.8183 | 0.01717 | 0.01717 |
| LOC102723589 | 0.8153 | 0.02702 | 0.02702 |
| LOC100506083 | 0.8144 | 0.008399 | 0.008399 |
| CLK4 | 0.8127 | 1.43E−10 | 1.43E−10 |
| PDE5A | 0.8121 | 0.01982 | 0.01982 |
| NGFR | 0.8097 | 0.005778 | 0.005778 |
| GPR135 | 0.8057 | 0.003204 | 0.003204 |
| TXLNB | 0.8051 | 0.0002951 | 0.0002951 |
| UNC5CL | 0.8031 | 0.01038 | 0.01038 |
| GP9 | 0.8031 | 0.01726 | 0.01726 |
| ELMO3 | 0.7963 | 2.35E−05 | 2.35E−05 |
| CCDC154 | 0.7952 | 0.0004243 | 0.0004243 |
| LOC101929280 | 0.7936 | 0.04057 | 0.04057 |
| ATP2B3 | 0.7931 | 0.0003836 | 0.0003836 |
| SLC22A20 | 0.7923 | 0.008079 | 0.008079 |
| LINC00959 | 0.7922 | 0.004591 | 0.004591 |
| PRSS53 | 0.79 | 5.15E−05 | 5.15E−05 |
| SCN8A | 0.7836 | 0.006734 | 0.006734 |
| AC093391.2 | 0.7835 | 0.006881 | 0.006881 |
| SARM1 | 0.783 | 0.0001099 | 0.0001099 |
| HHLA3 | 0.7796 | 6.30E−06 | 6.30E−06 |
| KIAA1324 | 0.7793 | 0.0008591 | 0.0008591 |
| AMH | 0.7789 | 0.002083 | 0.002083 |
| LOC100507373 | 0.7784 | 1.58E−05 | 1.58E−05 |
| LOC100506518 | 0.7779 | 7.18E−06 | 7.18E−06 |
| M1AP | 0.7754 | 0.007768 | 0.007768 |
| ZNF491 | 0.7713 | 0.007561 | 0.007561 |
| LOC100505771 | 0.7704 | 0.0008818 | 0.0008818 |
| GSDMB | 0.7698 | 0.001682 | 0.001682 |
| LOC101927974 | 0.7679 | 0.0007673 | 0.0007673 |
| TMEM254-AS1 | 0.767 | 0.004141 | 0.004141 |
| LTB4R2 | 0.7655 | 1.99E−06 | 1.99E−06 |
| LOC100506472 | 0.7649 | 0.001022 | 0.001022 |
| LOC101929243 | 0.7647 | 0.002408 | 0.002408 |
| LOC101928830 | 0.7631 | 0.01618 | 0.01618 |
| GPR113 | 0.7578 | 0.005797 | 0.005797 |
| LDLRAD4 | 0.7569 | 4.77E−07 | 4.77E−07 |
| CLEC12B | 0.7555 | 0.01879 | 0.01879 |
| C5orf56 | 0.7488 | 0.009988 | 0.009988 |
| PDPN | 0.7486 | 0.03511 | 0.03511 |
| GSTM2 | 0.7484 | 0.003808 | 0.003808 |
| LOC101929661 | 0.748 | 0.0032 | 0.0032 |
| EIF3CL | 0.7466 | 0.003876 | 0.003876 |
| ENHO | 0.7452 | 0.0004327 | 0.0004327 |
| LOC102724235 | 0.7375 | 0.003857 | 0.003857 |
| LINC00893 | 0.737 | 0.01581 | 0.01581 |
| APOLD1 | 0.7365 | 0.0001127 | 0.0001127 |
| SEC31B | 0.7359 | 5.72E−06 | 5.72E−06 |
| CACTIN-AS1 | 0.7321 | 0.004634 | 0.004634 |
| MDGA1 | 0.7307 | 0.002473 | 0.002473 |
| SIGLEC16 | 0.7305 | 0.0007117 | 0.0007117 |
| LOC102467073 | 0.73 | 0.02379 | 0.02379 |
| IPO9-AS1 | 0.7271 | 0.02734 | 0.02734 |
| LOC102724169 | 0.726 | 0.002057 | 0.002057 |
| LPAR2 | 0.7255 | 4.77E−09 | 4.77E−09 |
| SYTL2 | 0.7254 | 0.003306 | 0.003306 |
| TESPA1 | 0.724 | 0.002913 | 0.002913 |
| LOC728752 | 0.7225 | 0.003079 | 0.003079 |
| AP3B2 | 0.7192 | 0.0398 | 0.0398 |
| TP53I3 | 0.7187 | 4.41E−06 | 4.41E−06 |
| AP000330.8 | 0.7147 | 0.004826 | 0.004826 |
| LOC102723616 | 0.7122 | 0.03423 | 0.03423 |
| RP11-344E13.3 | 0.712 | 7.85E−13 | 7.85E−13 |
| TLR5 | 0.7065 | 0.01408 | 0.01408 |
| SLFNL1-AS1 | 0.7053 | 5.76E−06 | 5.76E−06 |
| GJC2 | 0.7031 | 0.04664 | 0.04664 |
| TPD52L1 | 0.7014 | 0.01284 | 0.01284 |
| CPNE5 | 0.7008 | 0.01143 | 0.01143 |
| LOC100507462 | 0.6976 | 0.01246 | 0.01246 |
| UCP3 | 0.6963 | 0.001122 | 0.001122 |
| SEMA3B | 0.6963 | 0.02005 | 0.02005 |
| GDPD3 | 0.695 | 0.01034 | 0.01034 |
| BTBD19 | 0.6939 | 0.02825 | 0.02825 |
| LOC100288069 | 0.6928 | 0.005851 | 0.005851 |
| DCAKD | 0.6901 | 1.03E−09 | 1.03E−09 |
| ID3 | 0.6893 | 0.0004751 | 0.0004751 |
| ADAM8 | 0.6886 | 8.35E−05 | 8.35E−05 |
| PITX2 | 0.6882 | 0.003134 | 0.003134 |
| ARTN | 0.6881 | 3.65E−06 | 3.65E−06 |
| LOC102724217 | 0.6865 | 0.001358 | 0.001358 |
| LAMC3 | 0.6855 | 0.002075 | 0.002075 |
| ABCC2 | 0.6855 | 0.009668 | 0.009668 |
| ORM1 | 0.6834 | 0.008792 | 0.008792 |
| TMEM191A | 0.6824 | 0.01585 | 0.01585 |
| LOC101929046 | 0.6794 | 0.007944 | 0.007944 |
| RP3-476K8.3 | 0.6781 | 0.0433 | 0.0433 |
| LOC102723714 | 0.6674 | 0.02358 | 0.02358 |
| TCAM1P | 0.6669 | 0.002562 | 0.002562 |
| CCDC155 | 0.6645 | 0.01486 | 0.01486 |
| TMPRSS13 | 0.6639 | 0.005115 | 0.005115 |
| BRICD5 | 0.6636 | 2.29E−05 | 2.29E−05 |
| NPFF | 0.6633 | 0.01125 | 0.01125 |
| WDFY3-AS2 | 0.6617 | 0.01072 | 0.01072 |
| PPP1R13L | 0.6598 | 7.29E−05 | 7.29E−05 |
| TMEM175 | 0.6594 | 1.27E−08 | 1.27E−08 |
| RNU86 | 0.6578 | 0.01054 | 0.01054 |
| STXBP5-AS1 | 0.6561 | 0.01142 | 0.01142 |
| SNX32 | 0.6555 | 0.001315 | 0.001315 |
| LOC102724248 | 0.652 | 0.003291 | 0.003291 |
| CDC42EP2 | 0.6518 | 0.003912 | 0.003912 |
| DEGS2 | 0.6503 | 0.02278 | 0.02278 |
| TMEM86B | 0.6481 | 5.29E−07 | 5.29E−07 |
| RCN3 | 0.6431 | 1.56E−05 | 1.56E−05 |
| LOC101927119 | 0.6408 | 0.02294 | 0.02294 |
| NOL3 | 0.6401 | 0.001321 | 0.001321 |
| LOC728715 | 0.6401 | 0.0204 | 0.0204 |
| WT1-AS | 0.6396 | 3.18E−06 | 3.18E−06 |
| VAMP5 | 0.6392 | 0.01861 | 0.01861 |
| CLDN15 | 0.6387 | 3.39E−07 | 3.39E−07 |
| LOC101927895 | 0.6379 | 0.04179 | 0.04179 |
| GALR2 | 0.6354 | 0.01227 | 0.01227 |
| C16orf86 | 0.6351 | 0.0002384 | 0.0002384 |
| LOC155060 | 0.6346 | 0.0001873 | 0.0001873 |
| DMPK | 0.6345 | 0.0005548 | 0.0005548 |
| RARRES3 | 0.6342 | 0.03888 | 0.03888 |
| APOE | 0.6335 | 2.01E−06 | 2.01E−06 |
| LINC00337 | 0.6329 | 0.02314 | 0.02314 |
| AURKB | 0.6324 | 2.14E−09 | 2.14E−09 |
| ARL17B | 0.6318 | 0.0209 | 0.0209 |
| METTL12 | 0.6315 | 0.0008087 | 0.0008087 |
| MIR1244-1 | 0.6309 | 0.0159 | 0.0159 |
| DNAJC27-AS1 | 0.6304 | 0.03354 | 0.03354 |
| PROB1 | 0.6297 | 0.0008142 | 0.0008142 |
| MAMDC4 | 0.628 | 4.07E−05 | 4.07E−05 |
| PPIEL | 0.6279 | 0.008493 | 0.008493 |
| PAQR6 | 0.6274 | 0.0001796 | 0.0001796 |
| RP11-307C12.11 | 0.6268 | 0.0004513 | 0.0004513 |
| HPD | 0.6253 | 0.03002 | 0.03002 |
| PSMG3-AS1 | 0.6249 | 4.09E−07 | 4.09E−07 |
| LOC644961 | 0.6222 | 0.00201 | 0.00201 |
| PTGER1 | 0.6218 | 0.01002 | 0.01002 |
| GIPR | 0.6177 | 0.01276 | 0.01276 |
| SPATA41 | 0.6171 | 0.03953 | 0.03953 |
| CLEC10A | 0.6157 | 0.01863 | 0.01863 |
| CATSPER3 | 0.6152 | 0.0194 | 0.0194 |
| ANKFN1 | 0.6129 | 0.002676 | 0.002676 |
| LOC101929747 | 0.6127 | 0.009365 | 0.009365 |
| RAPGEFL1 | 0.6118 | 4.27E−05 | 4.27E−05 |
| C9orf37 | 0.6108 | 4.05E−09 | 4.05E−09 |
| IL10RB-AS1 | 0.6103 | 0.001636 | 0.001636 |
| MYO7B | 0.6068 | 0.002185 | 0.002185 |
| C2orf48 | 0.606 | 0.004042 | 0.004042 |
| GAS6-AS1 | 0.6025 | 0.0001122 | 0.0001122 |
| CD302 | 0.6021 | 0.03042 | 0.03042 |
| DNAH1 | 0.6015 | 2.15E−08 | 2.15E−08 |
| MFI2-AS1 | 0.5997 | 0.003165 | 0.003165 |
| LOC101929112 | 0.5992 | 0.02087 | 0.02087 |
| SLFNL1 | 0.599 | 0.0305 | 0.0305 |
| ADAM19 | 0.5984 | 0.04367 | 0.04367 |
| HLA-F-AS1 | 0.5961 | 0.01589 | 0.01589 |
| MGC16142 | 0.5952 | 0.03968 | 0.03968 |
| TRIM72 | 0.5946 | 0.006274 | 0.006274 |
| BVES-AS1 | 0.5945 | 0.0179 | 0.0179 |
| SLC2A14 | 0.5944 | 0.01741 | 0.01741 |
| PCBP3 | 0.5937 | 0.003672 | 0.003672 |
| KCND1 | 0.5935 | 0.001082 | 0.001082 |
| TUBA1A | 0.5916 | 0.000171 | 0.000171 |
| FFAR4 | 0.5911 | 0.01248 | 0.01248 |
| PPP1R32 | 0.5902 | 0.01888 | 0.01888 |
| LOC101928710 | 0.5901 | 0.02826 | 0.02826 |
| CD101 | 0.5897 | 0.02758 | 0.02758 |
| LOC101929054 | 0.589 | 0.04092 | 0.04092 |
| DDB2 | 0.5883 | 9.92E−11 | 9.92E−11 |
| RP11-454P21.1 | 0.5869 | 0.03268 | 0.03268 |
| AC016629.8 | 0.586 | 2.85E−05 | 2.85E−05 |
| SNORD79 | 0.5858 | 0.01589 | 0.01589 |
| TSPEAR-AS1 | 0.5855 | 0.005867 | 0.005867 |
| LOC100133091 | 0.5851 | 0.001316 | 0.001316 |
| TMEM255A | −0.5904 | 0.001517 | 0.001517 |
| GPR3 | −0.5921 | 0.006117 | 0.006117 |
| CD163 | −0.5952 | 0.01736 | 0.01736 |
| FBXO15 | −0.5977 | 0.01129 | 0.01129 |
| SLC25A5-AS1 | −0.5981 | 0.01961 | 0.01961 |
| ZNF284 | −0.5982 | 0.008629 | 0.008629 |
| LOC100288102 | −0.6006 | 0.02688 | 0.02688 |
| GPR158 | −0.6058 | 0.0002466 | 0.0002466 |
| CCDC103 | −0.6115 | 0.00229 | 0.00229 |
| KCNH2 | −0.6125 | 0.003693 | 0.003693 |
| MBLAC1 | −0.6134 | 0.02149 | 0.02149 |
| KCNIP3 | −0.6151 | 0.02074 | 0.02074 |
| SEMG1 | −0.6203 | 0.002619 | 0.002619 |
| SERPINB2 | −0.6225 | 7.50E−13 | 7.50E−13 |
| ST13P4 | −0.623 | 0.03648 | 0.03648 |
| SYDE2 | −0.6236 | 0.0003263 | 0.0003263 |
| SPRED1 | −0.6296 | 0.009698 | 0.009698 |
| TTC4 | −0.6304 | 0.002673 | 0.002673 |
| SMA4 | −0.6311 | 0.04294 | 0.04294 |
| LOC102724098 | −0.6399 | 1.48E−05 | 1.48E−05 |
| TSKU | −0.6402 | 0.009829 | 0.009829 |
| SAMD13 | −0.6418 | 0.0007131 | 0.0007131 |
| PCOLCE2 | −0.6424 | 0.008973 | 0.008973 |
| RASSF8-AS1 | −0.6459 | 0.03274 | 0.03274 |
| RN7SL1 | −0.6652 | 0.004629 | 0.004629 |
| PAX8 | −0.6687 | 0.002062 | 0.002062 |
| SEMA3C | −0.6821 | 0.01336 | 0.01336 |
| RIMS2 | −0.6856 | 0.01222 | 0.01222 |
| LOC101927085 | −0.6948 | 0.002305 | 0.002305 |
| RPSAP9 | −0.6976 | 0.01868 | 0.01868 |
| SAMD5 | −0.6977 | 0.02336 | 0.02336 |
| SLCO2B1 | −0.6999 | 0.0029 | 0.0029 |
| IL8 | −0.7139 | 0.003093 | 0.003093 |
| RNU105A | −0.7199 | 0.02246 | 0.02246 |
| ZCWPW2 | −0.722 | 0.002292 | 0.002292 |
| RP11-261C10.3 | −0.7243 | 0.04782 | 0.04782 |
| RN7SK | −0.7805 | 0.04651 | 0.04651 |
| ZNF891 | −0.7965 | 0.002573 | 0.002573 |
| TMEM150A | −0.8108 | 0.0003505 | 0.0003505 |
| KRBA2 | −0.8123 | 0.009637 | 0.009637 |
| RP11-540A21.2 | −0.8218 | 0.0008734 | 0.0008734 |
| LOC101929814 | −0.8463 | 0.004974 | 0.004974 |
| LOC102724528 | −0.8481 | 3.02E−06 | 3.02E−06 |
| SIM1 | −0.8741 | 1.04E−05 | 1.04E−05 |
| MGC12916 | −0.9124 | 0.0002149 | 0.0002149 |
| ULK4P1 | −0.9282 | 0.03746 | 0.03746 |
| CHRFAM7A | −0.9429 | 0.004936 | 0.004936 |
| FLJ31945 | −0.9544 | 0.0005693 | 0.0005693 |
| IGFBP3 | −0.9606 | 0.001251 | 0.001251 |
| SLC7A11 | −0.9711 | 2.18E−10 | 2.18E−10 |
| LOC100130698 | −1.064 | 0.0002141 | 0.0002141 |
| SNORD3A | −1.579 | 0.002427 | 0.002427 |
| TNFAIP8L2-SCNM1 | −1.749 | 0.0001107 | 0.0001107 |
| RPPH1 | −1.864 | 0.002257 | 0.002257 |
| RNU2-2P | −3.126 | 0.001517 | 0.001517 |

TABLE 3
Differentially expressed genes with
PF-06650833 vs DMSO in THP1 cells

| Gene | Log(FC) | P Value | Adj P Value |
|---|---|---|---|
| CCL4 | 3.289 | 1.24E−07 | 1.24E−07 |
| IGFBP5 | 2.575 | 7.86E−08 | 7.86E−08 |
| CRABP2 | 2.494 | 4.22E−06 | 4.22E−06 |
| STC2 | 2.439 | 5.30E−08 | 5.30E−08 |
| COLEC12 | 2.26 | 1.28E−06 | 1.28E−06 |
| EGR2 | 2.256 | 1.16E−05 | 1.16E−05 |
| GFRA2 | 2.227 | 5.99E−10 | 5.99E−10 |
| SLC6A9 | 2.152 | 9.98E−07 | 9.98E−07 |
| EGR3 | 2.039 | 5.74E−09 | 5.74E−09 |
| C1QC | 2.005 | 1.06E−05 | 1.06E−05 |
| NTRK1 | 1.984 | 9.16E−08 | 9.16E−08 |
| LOC101927030 | 1.964 | 8.18E−06 | 8.18E−06 |
| GBP1 | 1.918 | 1.09E−06 | 1.09E−06 |
| LGMN | 1.841 | 2.28E−07 | 2.28E−07 |
| NR4A3 | 1.811 | 1.73E−05 | 1.73E−05 |
| PLXNA4 | 1.673 | 6.14E−10 | 6.14E−10 |
| CLEC7A | 1.667 | 1.05E−06 | 1.05E−06 |
| LOC100507424 | 1.616 | 0.0001903 | 0.0001903 |
| FERMT2 | 1.604 | 2.43E−05 | 2.43E−05 |
| ADM2 | 1.566 | 3.88E−09 | 3.88E−09 |
| PIP5KL1 | 1.553 | 4.42E−09 | 4.42E−09 |
| TNXB | 1.526 | 9.02E−08 | 9.02E−08 |
| IGFBP3 | 1.497 | 6.93E−07 | 6.93E−07 |
| CCL3 | 1.486 | 7.30E−07 | 7.30E−07 |
| ZNF205-AS1 | 1.477 | 0.0001292 | 0.0001292 |
| GJC2 | 1.449 | 1.68E−05 | 1.68E−05 |
| LRRC16B | 1.408 | 5.96E−05 | 5.96E−05 |
| CCDC176 | 1.4 | 6.43E−05 | 6.43E−05 |
| CD14 | 1.387 | 1.38E−08 | 1.38E−08 |
| SLC23A3 | 1.375 | 0.0002623 | 0.0002623 |
| UCN | 1.351 | 3.86E−06 | 3.86E−06 |
| SPOCD1 | 1.344 | 0.001497 | 0.001497 |
| LOC80154 | 1.337 | 0.0002532 | 0.0002532 |
| TDO2 | 1.319 | 6.24E−05 | 6.24E−05 |
| FSCN2 | 1.308 | 0.0001416 | 0.0001416 |
| ZFP36L1 | 1.303 | 4.00E−08 | 4.00E−08 |
| DMPK | 1.303 | 1.07E−07 | 1.07E−07 |
| C1QA | 1.3 | 3.79E−05 | 3.79E−05 |
| GUCA1B | 1.299 | 0.0001859 | 0.0001859 |
| SNX32 | 1.297 | 1.11E−07 | 1.11E−07 |
| ADAMTS4 | 1.291 | 6.04E−08 | 6.04E−08 |
| LILRB3 | 1.289 | 0.0003463 | 0.0003463 |
| CCDC183 | 1.264 | 4.23E−06 | 4.23E−06 |
| CXCL10 | 1.261 | 0.0001711 | 0.0001711 |
| NPTX1 | 1.239 | 7.49E−14 | 7.49E−14 |
| MMP9 | 1.236 | 3.51E−07 | 3.51E−07 |
| XKR5 | 1.227 | 0.0006901 | 0.0006901 |
| HSPA7 | 1.214 | 1.79E−05 | 1.79E−05 |
| LINC00959 | 1.198 | 6.99E−05 | 6.99E−05 |
| BZRAP1 | 1.197 | 4.14E−06 | 4.14E−06 |
| ST7-AS1 | 1.195 | 7.28E−05 | 7.28E−05 |
| CHAC1 | 1.187 | 3.06E−06 | 3.06E−06 |
| TUFT1 | 1.185 | 1.99E−07 | 1.99E−07 |
| MIR3187 | 1.178 | 0.004858 | 0.004858 |
| LAMB2P1 | 1.171 | 0.0006961 | 0.0006961 |
| DNAJC27-AS1 | 1.155 | 0.0007675 | 0.0007675 |
| TMEM9B-AS1 | 1.148 | 0.0001979 | 0.0001979 |
| LOC100134868 | 1.147 | 0.002577 | 0.002577 |
| PAPPA2 | 1.135 | 0.0002675 | 0.0002675 |
| FCRLB | 1.123 | 0.0006238 | 0.0006238 |
| ZP3 | 1.112 | 9.98E−06 | 9.98E−06 |
| GIMAP4 | 1.106 | 0.002989 | 0.002989 |
| RGS22 | 1.098 | 0.01446 | 0.01446 |
| LOC101929661 | 1.096 | 2.37E−05 | 2.37E−05 |
| ATP6V1B1 | 1.092 | 3.37E−05 | 3.37E−05 |
| DDIT4 | 1.091 | 2.13E−10 | 2.13E−10 |
| SPP1 | 1.09 | 2.56E−12 | 2.56E−12 |
| DND1 | 1.089 | 0.0009627 | 0.0009627 |
| OVGP1 | 1.087 | 0.0008256 | 0.0008256 |
| SMAGP | 1.085 | 0.0002803 | 0.0002803 |
| SNORA16A | 1.083 | 0.001672 | 0.001672 |
| FCGR2B | 1.077 | 1.67E−06 | 1.67E−06 |
| CEACAM19 | 1.074 | 1.18E−05 | 1.18E−05 |
| FAM86HP | 1.074 | 4.08E−05 | 4.08E−05 |
| BAIAP3 | 1.069 | 0.0008714 | 0.0008714 |
| COL4A2 | 1.067 | 4.70E−05 | 4.70E−05 |
| IL4I1 | 1.065 | 2.86E−08 | 2.86E−08 |
| SULF2 | 1.061 | 7.23E−07 | 7.23E−07 |
| C5AR1 | 1.057 | 8.07E−06 | 8.07E−06 |
| NPIPA8 | 1.054 | 0.02254 | 0.02254 |
| TNFAIP8L3 | 1.046 | 6.54E−05 | 6.54E−05 |
| LINC00852 | 1.042 | 0.002944 | 0.002944 |
| MERTK | 1.04 | 7.43E−06 | 7.43E−06 |
| RGPD5 | 1.034 | 0.01634 | 0.01634 |
| RGS20 | 1.033 | 7.87E−06 | 7.87E−06 |
| FBXO39 | 1.033 | 0.00132 | 0.00132 |
| ITGA10 | 1.021 | 0.01994 | 0.01994 |
| COL7A1 | 1.019 | 0.000471 | 0.000471 |
| DMWD | 1.01 | 3.67E−08 | 3.67E−08 |
| LOC284561 | 1.008 | 0.0009719 | 0.0009719 |
| TBX15 | 0.9995 | 3.82E−06 | 3.82E−06 |
| LOC100129924 | 0.999 | 0.001094 | 0.001094 |
| RGL1 | 0.9882 | 1.84E−05 | 1.84E−05 |
| LOC101928055 | 0.9875 | 6.24E−05 | 6.24E−05 |
| NPAS4 | 0.9873 | 0.0007027 | 0.0007027 |
| MAF | 0.9858 | 1.63E−06 | 1.63E−06 |
| LOC102467073 | 0.9792 | 0.00192 | 0.00192 |
| TNF | 0.9769 | 9.38E−07 | 9.38E−07 |
| MAEL | 0.9722 | 0.003201 | 0.003201 |
| LILRA6 | 0.9697 | 0.005032 | 0.005032 |
| MIR6859-1 | 0.9679 | 0.0006825 | 0.0006825 |
| DNHD1 | 0.9622 | 9.50E−07 | 9.50E−07 |
| GMPR | 0.9617 | 0.0002394 | 0.0002394 |
| VCAN | 0.9606 | 1.80E−12 | 1.80E−12 |
| LPAR6 | 0.9586 | 5.72E−07 | 5.72E−07 |
| ZNF660 | 0.9528 | 0.00693 | 0.00693 |
| ODF3B | 0.9517 | 3.42E−07 | 3.42E−07 |
| MAFB | 0.9482 | 4.70E−08 | 4.70E−08 |
| DLEU1 | 0.9476 | 3.48E−10 | 3.48E−10 |
| MIR1304 | 0.9441 | 0.003141 | 0.003141 |
| NOG | 0.9438 | 0.00126 | 0.00126 |
| TG | 0.9411 | 0.001149 | 0.001149 |
| ARTN | 0.94 | 3.99E−07 | 3.99E−07 |
| SNX22 | 0.9328 | 0.02218 | 0.02218 |
| C6orf164 | 0.9305 | 0.01886 | 0.01886 |
| TYMP | 0.9304 | 3.61E−13 | 3.61E−13 |
| C15orf62 | 0.9266 | 0.003237 | 0.003237 |
| KDM7A | 0.9238 | 1.14E−05 | 1.14E−05 |
| LOC102723539 | 0.9233 | 0.0111 | 0.0111 |
| FCGR2C | 0.9192 | 0.0006853 | 0.0006853 |
| TSPAN2 | 0.9171 | 8.43E−05 | 8.43E−05 |
| CCND1 | 0.9157 | 6.93E−07 | 6.93E−07 |
| GPR135 | 0.9122 | 0.001059 | 0.001059 |
| FAM229A | 0.9084 | 0.0007076 | 0.0007076 |
| CYBB | 0.9082 | 2.30E−13 | 2.30E−13 |
| SLC7A5P2 | 0.9068 | 0.0004882 | 0.0004882 |
| VSIG4 | 0.9068 | 0.0006297 | 0.0006297 |
| FBXL2 | 0.9045 | 0.03921 | 0.03921 |
| PRSS57 | 0.9042 | 0.001051 | 0.001051 |
| NDUFA4L2 | 0.9024 | 5.42E−06 | 5.42E−06 |
| LINC01137 | 0.9012 | 0.001017 | 0.001017 |
| SLC7A8 | 0.8957 | 1.84E−05 | 1.84E−05 |
| CCDC17 | 0.8953 | 0.02421 | 0.02421 |
| CYP27B1 | 0.8892 | 9.49E−05 | 9.49E−05 |
| ASIC3 | 0.8892 | 0.01145 | 0.01145 |
| FCER1G | 0.8867 | 2.20E−15 | 2.20E−15 |
| RPS6KA2 | 0.8831 | 2.86E−08 | 2.86E−08 |
| MDGA1 | 0.8814 | 0.0002049 | 0.0002049 |
| LOC100507403 | 0.878 | 0.002816 | 0.002816 |
| WNK4 | 0.8768 | 0.0007319 | 0.0007319 |
| ATP6AP1L | 0.8753 | 0.004168 | 0.004168 |
| HES4 | 0.8744 | 0.001723 | 0.001723 |
| ARHGAP31 | 0.8738 | 8.80E−08 | 8.80E−08 |
| GEM | 0.8734 | 0.001983 | 0.001983 |
| BSCL2 | 0.8729 | 0.001973 | 0.001973 |
| IFI44L | 0.87 | 1.34E−10 | 1.34E−10 |
| PTPRF | 0.869 | 3.26E−15 | 3.26E−15 |
| LYRM9 | 0.8667 | 6.84E−05 | 6.84E−05 |
| LOC102723975 | 0.8647 | 0.004304 | 0.004304 |
| FMNL2 | 0.8614 | 7.84E−08 | 7.84E−08 |
| LOC101928498 | 0.8614 | 4.48E−06 | 4.48E−06 |
| LOC100506100 | 0.8612 | 0.001425 | 0.001425 |
| FGD6 | 0.8584 | 0.02636 | 0.02636 |
| MAPK8IP2 | 0.8582 | 0.01267 | 0.01267 |
| MIR25 | 0.8562 | 0.006214 | 0.006214 |
| TMEM136 | 0.8558 | 0.0002695 | 0.0002695 |
| PAIP2B | 0.8549 | 0.001256 | 0.001256 |
| SNORD23 | 0.845 | 0.003847 | 0.003847 |
| LOC101929887 | 0.8434 | 0.01538 | 0.01538 |
| APOLD1 | 0.8405 | 8.73E−05 | 8.73E−05 |
| LOC101929264 | 0.8399 | 0.001093 | 0.001093 |
| LOC102723566 | 0.8345 | 0.01121 | 0.01121 |
| LOC101928061 | 0.8344 | 0.01315 | 0.01315 |
| MSTO2P | 0.8326 | 4.97E−05 | 4.97E−05 |
| PC | 0.8297 | 2.17E−06 | 2.17E−06 |
| AC011747.4 | 0.8271 | 0.000165 | 0.000165 |
| ARID3C | 0.8267 | 0.01059 | 0.01059 |
| CNFN | 0.8262 | 0.002023 | 0.002023 |
| HS3ST3A1 | 0.825 | 0.006772 | 0.006772 |
| DYSF | 0.8249 | 0.0002721 | 0.0002721 |
| KIF26A | 0.8244 | 0.0008009 | 0.0008009 |
| ELMO3 | 0.8236 | 1.90E−05 | 1.90E−05 |
| SLC29A2 | 0.8234 | 0.004801 | 0.004801 |
| AP3B2 | 0.8215 | 0.01752 | 0.01752 |
| FLJ38668 | 0.8194 | 0.00233 | 0.00233 |
| TM6SF1 | 0.8176 | 0.003407 | 0.003407 |
| BAI2 | 0.8125 | 0.004657 | 0.004657 |
| LOC100506548 | 0.8113 | 1.08E−11 | 1.08E−11 |
| WWTR1 | 0.8094 | 0.0003533 | 0.0003533 |
| TMEM249 | 0.8078 | 0.0003653 | 0.0003653 |
| AKR1C1 | 0.8022 | 2.15E−07 | 2.15E−07 |
| PPM1J | 0.8006 | 0.02053 | 0.02053 |
| RND3 | 0.799 | 0.0002383 | 0.0002383 |
| TGFB1I1 | 0.7986 | 6.41E−06 | 6.41E−06 |
| KDM4A | 0.7984 | 4.72E−15 | 4.72E−15 |
| ITIH4 | 0.7974 | 0.000136 | 0.000136 |
| NRP1 | 0.7956 | 1.08E−07 | 1.08E−07 |
| ADCY4 | 0.7949 | 3.97E−05 | 3.97E−05 |
| CCDC157 | 0.7949 | 0.0002938 | 0.0002938 |
| GCAT | 0.7929 | 3.78E−10 | 3.78E−10 |
| RUFY4 | 0.7909 | 0.0002859 | 0.0002859 |
| PPP1R32 | 0.7889 | 0.003896 | 0.003896 |
| DUSP6 | 0.7884 | 6.10E−11 | 6.10E−11 |
| FABP4 | 0.7874 | 0.00816 | 0.00816 |
| CSNK1E | 0.7843 | 5.28E−13 | 5.28E−13 |
| SLC25A34 | 0.7822 | 0.002542 | 0.002542 |
| AC009501.4 | 0.781 | 6.20E−07 | 6.20E−07 |
| ARHGEF33 | 0.7807 | 0.04726 | 0.04726 |
| TMOD2 | 0.7766 | 0.004161 | 0.004161 |
| MVK | 0.7763 | 4.29E−08 | 4.29E−08 |
| WDR63 | 0.7756 | 5.48E−08 | 5.48E−08 |
| GVINP1 | 0.7752 | 0.001471 | 0.001471 |
| C5orf56 | 0.7688 | 0.003978 | 0.003978 |
| DNAH6 | 0.7671 | 0.001252 | 0.001252 |
| IQCD | 0.7669 | 0.0004106 | 0.0004106 |
| RP1-234P15.4 | 0.7666 | 0.01162 | 0.01162 |
| LOC101928291 | 0.765 | 0.02055 | 0.02055 |
| POLN | 0.7629 | 0.00433 | 0.00433 |
| BAI3 | 0.76 | 0.00198 | 0.00198 |
| KLHL25 | 0.7591 | 0.001089 | 0.001089 |
| HAVCR2 | 0.7577 | 0.001538 | 0.001538 |
| FLJ32255 | 0.7549 | 5.30E−10 | 5.30E−10 |
| LOC101929540 | 0.7548 | 0.02358 | 0.02358 |
| ZDHHC1 | 0.7524 | 4.31E−05 | 4.31E−05 |
| RAB43 | 0.7504 | 0.002726 | 0.002726 |
| CATSPER2 | 0.749 | 0.003116 | 0.003116 |
| KHDRBS3 | 0.748 | 0.005767 | 0.005767 |
| GOLGA8I | 0.7478 | 0.0009563 | 0.0009563 |
| EFR3B | 0.7477 | 0.004828 | 0.004828 |
| MSMO1 | 0.7474 | 6.65E−11 | 6.65E−11 |
| GOLGA8M | 0.7431 | 1.11E−05 | 1.11E−05 |
| LOC101928236 | 0.7402 | 0.006828 | 0.006828 |
| ATF3 | 0.7392 | 0.0003015 | 0.0003015 |
| EPB41L3 | 0.7326 | 6.44E−05 | 6.44E−05 |
| MACROD2 | 0.732 | 0.04097 | 0.04097 |
| B4GALT3 | 0.7299 | 1.68E−13 | 1.68E−13 |
| LINC01133 | 0.7293 | 2.14E−05 | 2.14E−05 |
| VAMP5 | 0.7262 | 0.007717 | 0.007717 |
| CCDC168 | 0.726 | 0.02725 | 0.02725 |
| ADAMTS13 | 0.7258 | 0.0001203 | 0.0001203 |
| RAB30 | 0.7249 | 0.01874 | 0.01874 |
| WDR78 | 0.7248 | 0.0009827 | 0.0009827 |
| SNHG9 | 0.7207 | 0.0004372 | 0.0004372 |
| LOC100506127 | 0.7206 | 0.01007 | 0.01007 |
| PRSS53 | 0.7204 | 0.0002843 | 0.0002843 |
| TGFB3 | 0.716 | 0.001062 | 0.001062 |
| HHLA3 | 0.7146 | 5.04E−05 | 5.04E−05 |
| FAM106A | 0.7126 | 0.02782 | 0.02782 |
| ALDH1L2 | 0.7092 | 4.27E−10 | 4.27E−10 |
| ASNS | 0.7091 | 1.35E−08 | 1.35E−08 |
| CADM4 | 0.7086 | 0.01509 | 0.01509 |
| DLL1 | 0.7074 | 2.59E−06 | 2.59E−06 |
| GSDMB | 0.706 | 0.006157 | 0.006157 |
| GOLGA8S | 0.7043 | 0.001743 | 0.001743 |
| ABCC3 | 0.7039 | 0.005012 | 0.005012 |
| SLAMF8 | 0.7036 | 0.001235 | 0.001235 |
| ANKFN1 | 0.7003 | 0.001028 | 0.001028 |
| CCDC109B | 0.7003 | 0.0155 | 0.0155 |
| MYL9 | 0.7 | 2.77E−13 | 2.77E−13 |
| SERPINE2 | 0.6973 | 8.17E−06 | 8.17E−06 |
| HFM1 | 0.6957 | 0.00182 | 0.00182 |
| RNF150 | 0.6949 | 0.003711 | 0.003711 |
| LOC100507373 | 0.6923 | 0.0001831 | 0.0001831 |
| EGR4 | 0.6895 | 0.04072 | 0.04072 |
| TBC1D4 | 0.6891 | 7.07E−05 | 7.07E−05 |
| ZMYND12 | 0.6875 | 0.0004466 | 0.0004466 |
| TMEM191B | 0.6858 | 0.02669 | 0.02669 |
| ANXA2R | 0.684 | 1.67E−06 | 1.67E−06 |
| CLK1 | 0.6825 | 7.40E−10 | 7.40E−10 |
| FAM117A | 0.6817 | 6.87E−11 | 6.87E−11 |
| ULBP1 | 0.6817 | 0.001966 | 0.001966 |
| ARMC9 | 0.6809 | 2.54E−05 | 2.54E−05 |
| KDM4A-AS1 | 0.6797 | 1.13E−08 | 1.13E−08 |
| BRICD5 | 0.6785 | 2.10E−05 | 2.10E−05 |
| LOC100507316 | 0.6752 | 0.00374 | 0.00374 |
| ZNF837 | 0.6746 | 0.0003729 | 0.0003729 |
| DYRK1B | 0.674 | 0.000372 | 0.000372 |
| DSE | 0.6739 | 0.0001542 | 0.0001542 |
| RPS18P9 | 0.6739 | 0.005679 | 0.005679 |
| LOC101928891 | 0.6706 | 1.85E−05 | 1.85E−05 |
| MYCBPAP | 0.6704 | 0.0001176 | 0.0001176 |
| DUSP8 | 0.6703 | 0.004321 | 0.004321 |
| EFCAB12 | 0.6683 | 0.009862 | 0.009862 |
| CNIH3 | 0.6672 | 0.01 | 0.01 |
| SAP25 | 0.6664 | 0.0004155 | 0.0004155 |
| LIME1 | 0.6643 | 2.32E−06 | 2.32E−06 |
| EPSTI1 | 0.6635 | 3.73E−06 | 3.73E−06 |
| LOC644961 | 0.6627 | 0.001475 | 0.001475 |
| RABL2A | 0.662 | 7.94E−07 | 7.94E−07 |
| GYPC | 0.6616 | 3.02E−11 | 3.02E−11 |
| LOC100128494 | 0.6601 | 0.01518 | 0.01518 |
| RAB7B | 0.6594 | 3.26E−11 | 3.26E−11 |
| FBXO36 | 0.6581 | 0.008048 | 0.008048 |
| PDGFA | 0.658 | 0.0001519 | 0.0001519 |
| CMTM1 | 0.6576 | 0.0004042 | 0.0004042 |
| DEFB1 | 0.6573 | 4.35E−07 | 4.35E−07 |
| MGC16142 | 0.6573 | 0.02766 | 0.02766 |
| HOXA10-AS | 0.6571 | 2.44E−07 | 2.44E−07 |
| SLC4A5 | 0.6567 | 0.02946 | 0.02946 |
| SEC31B | 0.6526 | 0.000611 | 0.000611 |
| FAM86B3P | 0.652 | 0.0002163 | 0.0002163 |
| PHGDH | 0.6517 | 3.35E−11 | 3.35E−11 |
| LOC102724169 | 0.6507 | 0.002849 | 0.002849 |
| CDH15 | 0.6506 | 0.01514 | 0.01514 |
| DTX4 | 0.6489 | 1.19E−08 | 1.19E−08 |
| LOC102724200 | 0.6477 | 0.0002363 | 0.0002363 |
| TMEM120B | 0.647 | 2.00E−07 | 2.00E−07 |
| LY6E | 0.6468 | 1.03E−09 | 1.03E−09 |
| FBXW9 | 0.6445 | 5.11E−07 | 5.11E−07 |
| SUGT1P1 | 0.6445 | 0.01143 | 0.01143 |
| CBS | 0.6442 | 0.0003749 | 0.0003749 |
| BPHL | 0.6436 | 0.001821 | 0.001821 |
| MAPK8IP3 | 0.6418 | 3.53E−09 | 3.53E−09 |
| WNT11 | 0.6411 | 0.018 | 0.018 |
| LOC101060264 | 0.6408 | 0.008278 | 0.008278 |
| LOC100996255 | 0.6406 | 6.04E−05 | 6.04E−05 |
| HEXDC | 0.6399 | 4.40E−08 | 4.40E−08 |
| DRP2 | 0.6398 | 0.005272 | 0.005272 |
| OGDHL | 0.638 | 0.0006565 | 0.0006565 |
| SEMA3B | 0.638 | 0.02725 | 0.02725 |
| OAS3 | 0.6367 | 1.31E−10 | 1.31E−10 |
| CAPS | 0.6367 | 0.002033 | 0.002033 |
| POU6F1 | 0.6362 | 0.0003754 | 0.0003754 |
| HOXA11-AS | 0.6352 | 1.13E−07 | 1.13E−07 |
| WIPI1 | 0.6348 | 0.01021 | 0.01021 |
| BEX1 | 0.6345 | 4.59E−11 | 4.59E−11 |
| ECEL1 | 0.634 | 0.002337 | 0.002337 |
| HIP1R | 0.6336 | 1.60E−06 | 1.60E−06 |
| EFHC1 | 0.6331 | 6.68E−05 | 6.68E−05 |
| AJAP1 | 0.6325 | 3.38E−06 | 3.38E−06 |
| ZNF491 | 0.6315 | 0.03485 | 0.03485 |
| SPTBN5 | 0.6308 | 0.0004272 | 0.0004272 |
| MVD | 0.6304 | 1.59E−10 | 1.59E−10 |
| DHCR7 | 0.6274 | 3.58E−09 | 3.58E−09 |
| LOC284889 | 0.6273 | 0.0009566 | 0.0009566 |
| CSNK1D | 0.6268 | 2.55E−12 | 2.55E−12 |
| NUTM2D | 0.6255 | 0.009197 | 0.009197 |
| LOC155060 | 0.6248 | 0.000533 | 0.000533 |
| LOC100133091 | 0.624 | 0.001533 | 0.001533 |
| MROH6 | 0.623 | 0.0001424 | 0.0001424 |
| KDELC1 | 0.6226 | 1.16E−07 | 1.16E−07 |
| VMAC | 0.6223 | 2.60E−05 | 2.60E−05 |
| AMH | 0.6216 | 0.01002 | 0.01002 |
| KCNMB1 | 0.6206 | 3.05E−09 | 3.05E−09 |
| ZNF862 | 0.6201 | 1.81E−08 | 1.81E−08 |
| POMC | 0.6194 | 2.47E−05 | 2.47E−05 |
| LOC101928069 | 0.6192 | 0.03161 | 0.03161 |
| SLCO2B1 | 0.6179 | 0.00231 | 0.00231 |
| EPPK1 | 0.6174 | 0.001133 | 0.001133 |
| LTB4R2 | 0.6156 | 6.22E−05 | 6.22E−05 |
| LINC00923 | 0.6156 | 0.0392 | 0.0392 |
| SNORD47 | 0.6141 | 0.0001853 | 0.0001853 |
| CLK4 | 0.614 | 2.48E−07 | 2.48E−07 |
| FAM195B | 0.6137 | 3.78E−09 | 3.78E−09 |
| PPIEL | 0.6131 | 0.009004 | 0.009004 |
| YTHDF3-AS1 | 0.6129 | 0.01483 | 0.01483 |
| KCNJ2 | 0.6121 | 0.01611 | 0.01611 |
| SH3BP5 | 0.612 | 1.33E−08 | 1.33E−08 |
| KCND1 | 0.6111 | 0.001184 | 0.001184 |
| HMOX1 | 0.6105 | 3.59E−05 | 3.59E−05 |
| WDR31 | 0.6097 | 0.0002511 | 0.0002511 |
| MTSS1 | 0.6091 | 0.002149 | 0.002149 |
| C11orf96 | 0.609 | 0.001221 | 0.001221 |
| P2RY11 | 0.6089 | 0.0003097 | 0.0003097 |
| THBS1 | 0.6067 | 0.002686 | 0.002686 |
| LINC00936 | 0.6067 | 0.01834 | 0.01834 |
| TBC1D16 | 0.6053 | 6.59E−09 | 6.59E−09 |
| RRNAD1 | 0.6046 | 1.44E−09 | 1.44E−09 |
| PSMG3-AS1 | 0.6046 | 6.89E−07 | 6.89E−07 |
| MIRLET7BHG | 0.6031 | 0.000963 | 0.000963 |
| PLEKHO1 | 0.6026 | 8.60E−10 | 8.60E−10 |
| ARHGAP39 | 0.6023 | 0.0009057 | 0.0009057 |
| FAM86JP | 0.6016 | 0.002215 | 0.002215 |
| BEND6 | 0.6013 | 0.0001593 | 0.0001593 |
| VEGFA | 0.6012 | 2.00E−09 | 2.00E−09 |
| FHL2 | 0.6008 | 2.29E−07 | 2.29E−07 |
| FLJ23867 | 0.6003 | 2.69E−07 | 2.69E−07 |
| C14orf79 | 0.5996 | 1.31E−05 | 1.31E−05 |
| CYR61 | 0.5968 | 0.003388 | 0.003388 |
| TMEM243 | 0.5958 | 0.0004383 | 0.0004383 |
| ZNF880 | 0.5954 | 0.0175 | 0.0175 |
| SNORA61 | 0.5952 | 0.006134 | 0.006134 |
| RHPN1 | 0.5949 | 2.31E−06 | 2.31E−06 |
| ZKSCAN7 | 0.5948 | 0.0004542 | 0.0004542 |
| YJEFN3 | 0.5945 | 0.0002329 | 0.0002329 |
| RP11-890B15.2 | 0.5938 | 0.000124 | 0.000124 |
| BDNF-AS | 0.5938 | 0.001856 | 0.001856 |
| TSGA10 | 0.5927 | 0.006394 | 0.006394 |
| LOC100652768 | 0.5921 | 0.03597 | 0.03597 |
| MSH5-SAPCD1 | 0.5918 | 0.03224 | 0.03224 |
| NOTUM | 0.5915 | 0.0001975 | 0.0001975 |
| GOLGA8K | 0.5909 | 0.001288 | 0.001288 |
| HELZ2 | 0.5883 | 2.47E−09 | 2.47E−09 |
| AQP1 | 0.5878 | 0.02184 | 0.02184 |
| SGSH | 0.5856 | 0.008507 | 0.008507 |
| LOC102724256 | −0.5851 | 1.74E−05 | 1.74E−05 |
| C1orf186 | −0.5855 | 4.08E−05 | 4.08E−05 |
| EVA1C | −0.5859 | 0.01363 | 0.01363 |
| ANKRD35 | −0.5869 | 5.53E−07 | 5.53E−07 |
| FAM150A | −0.5871 | 0.004085 | 0.004085 |
| LOC100129203 | −0.5871 | 0.009145 | 0.009145 |
| VIM-AS1 | −0.5879 | 0.0009357 | 0.0009357 |
| SLC17A5 | −0.5886 | 1.26E−09 | 1.26E−09 |
| PROX2 | −0.5893 | 0.01732 | 0.01732 |
| ARHGAP25 | −0.5895 | 1.33E−07 | 1.33E−07 |
| LOC101929545 | −0.5904 | 0.005401 | 0.005401 |
| TMEM45A | −0.5913 | 0.0001047 | 0.0001047 |
| IQGAP2 | −0.5919 | 1.22E−07 | 1.22E−07 |
| SYTL1 | −0.5922 | 0.0001249 | 0.0001249 |
| MPZ | −0.5945 | 0.0002494 | 0.0002494 |
| LOC102723883 | −0.5967 | 0.02617 | 0.02617 |
| CD300LB | −0.5972 | 0.0005345 | 0.0005345 |
| FOS | −0.5974 | 2.52E−06 | 2.52E−06 |
| C1QL4 | −0.5987 | 0.02055 | 0.02055 |
| ARRDC3-AS1 | −0.5996 | 0.01169 | 0.01169 |
| NCALD | −0.6 | 1.38E−06 | 1.38E−06 |
| FAM161B | −0.6003 | 0.000525 | 0.000525 |
| TACC2 | −0.6004 | 5.64E−06 | 5.64E−06 |
| HSPA1L | −0.6007 | 0.0004268 | 0.0004268 |
| SWT1 | −0.6012 | 0.006825 | 0.006825 |
| LHFPL2 | −0.6019 | 2.06E−05 | 2.06E−05 |
| CCDC78 | −0.6022 | 8.66E−05 | 8.66E−05 |
| GAPT | −0.6025 | 6.01E−10 | 6.01E−10 |
| C1orf226 | −0.604 | 1.82E−06 | 1.82E−06 |
| SYN3 | −0.6041 | 0.005724 | 0.005724 |
| CACNA2D3 | −0.6049 | 1.26E−08 | 1.26E−08 |
| SLC2A3 | −0.6056 | 0.0006029 | 0.0006029 |
| ZNF836 | −0.606 | 0.0006991 | 0.0006991 |
| PDCD4 | −0.6069 | 7.27E−11 | 7.27E−11 |
| GIPC3 | −0.6076 | 1.00E−09 | 1.00E−09 |
| LOC81691 | −0.6095 | 7.27E−09 | 7.27E−09 |
| RPSAP9 | −0.6104 | 0.03111 | 0.03111 |
| SIM1 | −0.6107 | 0.0004076 | 0.0004076 |
| HYDIN2 | −0.6113 | 0.02136 | 0.02136 |
| LOC101929119 | −0.6138 | 0.03086 | 0.03086 |
| TMEM238 | −0.6139 | 0.04929 | 0.04929 |
| SERPINA1 | −0.615 | 0.0004516 | 0.0004516 |
| FOSL1 | −0.6175 | 1.52E−06 | 1.52E−06 |
| DGKG | −0.6202 | 4.66E−08 | 4.66E−08 |
| BMP8B | −0.6206 | 2.17E−13 | 2.17E−13 |
| SNX10 | −0.6217 | 1.75E−08 | 1.75E−08 |
| SNORA73A | −0.6228 | 0.0167 | 0.0167 |
| RGS3 | −0.6231 | 0.001326 | 0.001326 |
| PSTPIP1 | −0.6238 | 1.12E−08 | 1.12E−08 |
| KREMEN2 | −0.6258 | 0.001855 | 0.001855 |
| RSPO1 | −0.6269 | 0.01096 | 0.01096 |
| ZNF630 | −0.6307 | 0.01635 | 0.01635 |
| SCIMP | −0.6308 | 0.003438 | 0.003438 |
| ASGR2 | −0.631 | 0.0001283 | 0.0001283 |
| LOC100093631 | −0.631 | 0.0004864 | 0.0004864 |
| LOC101927841 | −0.6312 | 0.0001154 | 0.0001154 |
| MPO | −0.6316 | 0.0001052 | 0.0001052 |
| SLC6A11 | −0.6327 | 0.00349 | 0.00349 |
| C1orf228 | −0.634 | 2.89E−10 | 2.89E−10 |
| PCED1B-AS1 | −0.6355 | 3.29E−06 | 3.29E−06 |
| MUC19 | −0.6358 | 0.001935 | 0.001935 |
| EVI2B | −0.6362 | 3.17E−12 | 3.17E−12 |
| TLR1 | −0.6365 | 0.0009513 | 0.0009513 |
| NHSL2 | −0.639 | 1.40E−08 | 1.40E−08 |
| FMO5 | −0.639 | 4.09E−05 | 4.09E−05 |
| BPI | −0.6416 | 0.04729 | 0.04729 |
| AQP3 | −0.6432 | 2.37E−05 | 2.37E−05 |
| C19orf59 | −0.6476 | 1.28E−09 | 1.28E−09 |
| S1PR3 | −0.6476 | 1.48E−07 | 1.48E−07 |
| LDOC1 | −0.6494 | 0.001256 | 0.001256 |
| APOBR | −0.6497 | 4.45E−11 | 4.45E−11 |
| S1PR5 | −0.6503 | 0.0001619 | 0.0001619 |
| LOC100507642 | −0.6508 | 4.51E−06 | 4.51E−06 |
| SLC9A7 | −0.6539 | 1.54E−05 | 1.54E−05 |
| LOC339862 | −0.6552 | 0.007553 | 0.007553 |
| CALHM2 | −0.6575 | 8.40E−05 | 8.40E−05 |
| NHLRC3 | −0.66 | 5.18E−07 | 5.18E−07 |
| C3 | −0.6607 | 4.52E−12 | 4.52E−12 |
| GPR35 | −0.6621 | 8.54E−05 | 8.54E−05 |
| KLHL30 | −0.6632 | 0.0002329 | 0.0002329 |
| GNG12 | −0.6632 | 0.003494 | 0.003494 |
| FAM111B | −0.6646 | 7.41E−11 | 7.41E−11 |
| PODNL1 | −0.6661 | 0.0132 | 0.0132 |
| IL16 | −0.667 | 3.14E−07 | 3.14E−07 |
| ADPGK-AS1 | −0.6683 | 0.003957 | 0.003957 |
| TREM1 | −0.6699 | 0.00879 | 0.00879 |
| APOC1 | −0.67 | 1.43E−08 | 1.43E−08 |
| CENPH | −0.6701 | 1.21E−09 | 1.21E−09 |
| MTMR11 | −0.6705 | 1.38E−06 | 1.38E−06 |
| ALDH1A3 | −0.6705 | 0.009436 | 0.009436 |
| SETBP1 | −0.6709 | 0.0001633 | 0.0001633 |
| ACVR1C | −0.6723 | 0.001876 | 0.001876 |
| TRIM72 | −0.6733 | 0.005988 | 0.005988 |
| CCL23 | −0.6739 | 3.19E−07 | 3.19E−07 |
| PAX8 | −0.674 | 0.002641 | 0.002641 |
| C19orf35 | −0.675 | 0.0001515 | 0.0001515 |
| KCNA3 | −0.6759 | 1.74E−08 | 1.74E−08 |
| LINC01094 | −0.6762 | 0.01506 | 0.01506 |
| PRTN3 | −0.6776 | 3.69E−11 | 3.69E−11 |
| THBS4 | −0.6785 | 7.11E−07 | 7.11E−07 |
| NMNAT2 | −0.6786 | 0.01318 | 0.01318 |
| FAS | −0.6791 | 0.005757 | 0.005757 |
| TNFSF10 | −0.6794 | 1.32E−07 | 1.32E−07 |
| IL1R1 | −0.6855 | 0.004712 | 0.004712 |
| MPEG1 | −0.6879 | 8.63E−11 | 8.63E−11 |
| KIF21B | −0.6912 | 2.75E−10 | 2.75E−10 |
| FAT4 | −0.6913 | 0.03983 | 0.03983 |
| C20orf197 | −0.6914 | 0.0002667 | 0.0002667 |
| TNS1 | −0.6915 | 5.01E−09 | 5.01E−09 |
| HOXA4 | −0.6934 | 0.01335 | 0.01335 |
| ZNF385A | −0.6954 | 5.44E−13 | 5.44E−13 |
| TXNDC5 | −0.6972 | 0.003239 | 0.003239 |
| MIR223 | −0.6973 | 0.007621 | 0.007621 |
| DBNDD1 | −0.7011 | 0.008893 | 0.008893 |
| ARHGAP42 | −0.7014 | 0.003632 | 0.003632 |
| EMILIN1 | −0.7021 | 0.0004212 | 0.0004212 |
| SLC29A4 | −0.7026 | 4.28E−07 | 4.28E−07 |
| C20orf112 | −0.7028 | 5.80E−10 | 5.80E−10 |
| SYT11 | −0.7029 | 0.002233 | 0.002233 |
| CORO2A | −0.7032 | 1.44E−08 | 1.44E−08 |
| TNNT1 | −0.7036 | 8.01E−10 | 8.01E−10 |
| PTPRO | −0.7068 | 4.32E−09 | 4.32E−09 |
| HK3 | −0.7069 | 2.21E−09 | 2.21E−09 |
| AGT | −0.7076 | 5.29E−12 | 5.29E−12 |
| NPAS1 | −0.7099 | 0.0005322 | 0.0005322 |
| COL15A1 | −0.7101 | 0.0001607 | 0.0001607 |
| GUCY1A2 | −0.7109 | 0.01957 | 0.01957 |
| DISC1 | −0.711 | 0.008048 | 0.008048 |
| BLNK | −0.7123 | 0.005472 | 0.005472 |
| PPP1R3G | −0.7143 | 3.77E−09 | 3.77E−09 |
| LOC101929017 | −0.7157 | 0.004197 | 0.004197 |
| ROM1 | −0.7161 | 0.002756 | 0.002756 |
| ITGA7 | −0.7201 | 1.20E−05 | 1.20E−05 |
| EPHB6 | −0.7205 | 9.99E−08 | 9.99E−08 |
| NLRC4 | −0.7207 | 0.0001096 | 0.0001096 |
| HIVEP1 | −0.721 | 0.0002165 | 0.0002165 |
| TCN2 | −0.7222 | 0.003808 | 0.003808 |
| DYNC1I1 | −0.7256 | 0.02718 | 0.02718 |
| ZDHHC2 | −0.7271 | 1.99E−10 | 1.99E−10 |
| LINC00977 | −0.7277 | 0.0001526 | 0.0001526 |
| JADE2 | −0.7279 | 3.25E−12 | 3.25E−12 |
| AOAH | −0.7284 | 7.53E−06 | 7.53E−06 |
| ZRANB2 | −0.7305 | 6.36E−10 | 6.36E−10 |
| CCDC103 | −0.7315 | 0.0007288 | 0.0007288 |
| SPTB | −0.7316 | 0.00909 | 0.00909 |
| MAPK13 | −0.732 | 6.22E−06 | 6.22E−06 |
| CIB3 | −0.7332 | 0.01446 | 0.01446 |
| MAP3K12 | −0.7385 | 0.005251 | 0.005251 |
| MRVI1 | −0.7407 | 0.003672 | 0.003672 |
| HVCN1 | −0.7409 | 9.13E−08 | 9.13E−08 |
| ST13P4 | −0.746 | 0.00448 | 0.00448 |
| RRM2 | −0.7461 | 2.95E−13 | 2.95E−13 |
| SAMD15 | −0.7474 | 0.005549 | 0.005549 |
| ZNF804A | −0.7477 | 2.61E−09 | 2.61E−09 |
| POU2F2 | −0.7486 | 2.76E−08 | 2.76E−08 |
| SLAMF6 | −0.7493 | 2.12E−05 | 2.12E−05 |
| LINC01140 | −0.7494 | 0.0004332 | 0.0004332 |
| FAM171A1 | −0.7497 | 0.0006271 | 0.0006271 |
| TMEM38A | −0.7514 | 0.00062 | 0.00062 |
| ANK1 | −0.7531 | 0.004581 | 0.004581 |
| FLT3 | −0.7537 | 5.66E−12 | 5.66E−12 |
| NAIP | −0.756 | 1.18E−06 | 1.18E−06 |
| KCNN1 | −0.7562 | 0.005132 | 0.005132 |
| DKK2 | −0.7603 | 9.45E−07 | 9.45E−07 |
| PINLYP | −0.7608 | 0.0001727 | 0.0001727 |
| HAPLN3 | −0.761 | 0.008535 | 0.008535 |
| PDE7B | −0.7615 | 1.88E−08 | 1.88E−08 |
| LYZ | −0.7629 | 1.85E−14 | 1.85E−14 |
| PPFIA4 | −0.7651 | 6.67E−07 | 6.67E−07 |
| FAM83B | −0.7654 | 0.001277 | 0.001277 |
| RP11-344B5.2 | −0.7679 | 0.005385 | 0.005385 |
| TEC | −0.7683 | 4.90E−11 | 4.90E−11 |
| ATL1 | −0.7704 | 0.002434 | 0.002434 |
| PDGFD | −0.7728 | 1.93E−08 | 1.93E−08 |
| SOX1 | −0.7735 | 0.005021 | 0.005021 |
| SSPO | −0.7746 | 0.0004629 | 0.0004629 |
| RP11-12A2.3 | −0.7755 | 0.002975 | 0.002975 |
| CXCR2 | −0.7757 | 3.66E−10 | 3.66E−10 |
| LOC102723375 | −0.7777 | 0.01399 | 0.01399 |
| NAPSB | −0.7786 | 1.09E−11 | 1.09E−11 |
| SUCNR1 | −0.7829 | 0.004753 | 0.004753 |
| C1orf200 | −0.7835 | 1.82E−06 | 1.82E−06 |
| SIGLEC7 | −0.7835 | 0.00623 | 0.00623 |
| TGFBR1 | −0.7837 | 1.39E−12 | 1.39E−12 |
| LOC441081 | −0.7841 | 0.01311 | 0.01311 |
| LOC101926967 | −0.7845 | 0.005217 | 0.005217 |
| CAMSAP2 | −0.7856 | 1.74E−09 | 1.74E−09 |
| EPS8L3 | −0.7859 | 0.001515 | 0.001515 |
| TTBK1 | −0.7874 | 0.005246 | 0.005246 |
| ARAP3 | −0.7895 | 3.88E−05 | 3.88E−05 |
| ZSCANSA | −0.7934 | 6.21E−07 | 6.21E−07 |
| TEAD3 | −0.7971 | 5.93E−07 | 5.93E−07 |
| ANK3 | −0.7983 | 0.002059 | 0.002059 |
| LOC102724740 | −0.7986 | 0.0001148 | 0.0001148 |
| ST8SIA5 | −0.7999 | 0.001964 | 0.001964 |
| FAM212A | −0.8011 | 2.95E−06 | 2.95E−06 |
| PPP1R1C | −0.8015 | 0.005555 | 0.005555 |
| STAP1 | −0.8025 | 0.0005231 | 0.0005231 |
| C19orf38 | −0.8036 | 1.59E−06 | 1.59E−06 |
| CDA | −0.8048 | 0.00027 | 0.00027 |
| SLA2 | −0.8054 | 0.001515 | 0.001515 |
| CD84 | −0.806 | 3.86E−13 | 3.86E−13 |
| TMC8 | −0.8072 | 2.07E−10 | 2.07E−10 |
| FCGR1C | −0.8082 | 1.80E−05 | 1.80E−05 |
| GPA33 | −0.8087 | 1.24E−10 | 1.24E−10 |
| RIMS2 | −0.8103 | 0.01082 | 0.01082 |
| MPP7 | −0.8145 | 0.0001462 | 0.0001462 |
| SCAMP5 | −0.8149 | 9.11E−11 | 9.11E−11 |
| ACY3 | −0.8185 | 0.01955 | 0.01955 |
| IPCEF1 | −0.8187 | 1.20E−12 | 1.20E−12 |
| LOC101927402 | −0.8192 | 3.66E−06 | 3.66E−06 |
| LOXHD1 | −0.8198 | 4.49E−06 | 4.49E−06 |
| PDCD4-AS1 | −0.822 | 0.002122 | 0.002122 |
| FXYD6 | −0.8254 | 0.0009757 | 0.0009757 |
| TBXA2R | −0.8265 | 0.0005963 | 0.0005963 |
| NLRC3 | −0.8267 | 3.14E−05 | 3.14E−05 |
| CDC42EP4 | −0.828 | 1.96E−08 | 1.96E−08 |
| SYNC | −0.8315 | 0.0001209 | 0.0001209 |
| TNFSF14 | −0.8321 | 8.11E−09 | 8.11E−09 |
| LOC101927727 | −0.8344 | 5.29E−05 | 5.29E−05 |
| HERC2P3 | −0.8359 | 0.0009494 | 0.0009494 |
| LMTK3 | −0.8375 | 1.17E−05 | 1.17E−05 |
| FCGR1A | −0.8405 | 4.97E−13 | 4.97E−13 |
| PCDH12 | −0.8428 | 4.85E−05 | 4.85E−05 |
| CASS4 | −0.8445 | 5.60E−06 | 5.60E−06 |
| TBC1D10C | −0.8446 | 0.005701 | 0.005701 |
| ZNF280A | −0.8461 | 0.01084 | 0.01084 |
| NUAK1 | −0.8471 | 0.003439 | 0.003439 |
| ST3GAL6 | −0.8472 | 8.57E−10 | 8.57E−10 |
| UTF1 | −0.8522 | 0.0004818 | 0.0004818 |
| ZNF467 | −0.8564 | 6.61E−08 | 6.61E−08 |
| PLEKHA7 | −0.8616 | 0.001675 | 0.001675 |
| HGF | −0.8618 | 7.88E−06 | 7.88E−06 |
| C1orf54 | −0.8667 | 7.66E−10 | 7.66E−10 |
| GALNT3 | −0.8672 | 0.00297 | 0.00297 |
| CD244 | −0.8676 | 4.90E−10 | 4.90E−10 |
| KIAA1644 | −0.8707 | 6.06E−05 | 6.06E−05 |
| PTAFR | −0.876 | 3.23E−08 | 3.23E−08 |
| PANX2 | −0.8769 | 0.0001463 | 0.0001463 |
| ADRB2 | −0.8797 | 4.36E−05 | 4.36E−05 |
| TMEM156 | −0.8837 | 0.001552 | 0.001552 |
| RNU105A | −0.889 | 0.01168 | 0.01168 |
| COL24A1 | −0.8894 | 0.0001137 | 0.0001137 |
| ADAMDEC1 | −0.8915 | 0.0004552 | 0.0004552 |
| SPNS3 | −0.8934 | 5.02E−09 | 5.02E−09 |
| ITGAL | −0.8979 | 6.60E−13 | 6.60E−13 |
| MILR1 | −0.9008 | 2.84E−09 | 2.84E−09 |
| TNFSF13 | −0.9069 | 4.70E−05 | 4.70E−05 |
| GPNMB | −0.9098 | 1.07E−12 | 1.07E−12 |
| MMP19 | −0.9114 | 0.001211 | 0.001211 |
| TSPAN32 | −0.9149 | 2.64E−07 | 2.64E−07 |
| TNNI2 | −0.9152 | 0.001752 | 0.001752 |
| RAB37 | −0.9156 | 5.57E−06 | 5.57E−06 |
| LOC102724017 | −0.9194 | 0.00284 | 0.00284 |
| HSPB7 | −0.9221 | 6.71E−08 | 6.71E−08 |
| KCNIP3 | −0.9234 | 0.002365 | 0.002365 |
| TAF9B | −0.9236 | 6.12E−10 | 6.12E−10 |
| GPR18 | −0.9263 | 8.56E−09 | 8.56E−09 |
| PNOC | −0.9344 | 0.001406 | 0.001406 |
| IL12RB1 | −0.9377 | 2.39E−08 | 2.39E−08 |
| ZNF536 | −0.9396 | 0.00355 | 0.00355 |
| SLPI | −0.9448 | 3.71E−10 | 3.71E−10 |
| METTL7A | −0.9471 | 5.92E−08 | 5.92E−08 |
| MCTP1 | −0.9488 | 2.40E−10 | 2.40E−10 |
| ARHGAP24 | −0.9524 | 0.0001124 | 0.0001124 |
| AIM1 | −0.9582 | 9.57E−13 | 9.57E−13 |
| CLEC12A | −0.9606 | 6.16E−07 | 6.16E−07 |
| LOC101928429 | −0.9638 | 2.78E−11 | 2.78E−11 |
| ARID5B | −0.9649 | 8.35E−06 | 8.35E−06 |
| IKZF3 | −0.9658 | 0.0005995 | 0.0005995 |
| LOC100653515 | −0.9658 | 0.002789 | 0.002789 |
| ANXA1 | −0.9704 | 6.27E−14 | 6.27E−14 |
| MYBPH | −0.9733 | 2.43E−12 | 2.43E−12 |
| SLAMF1 | −0.9814 | 0.0002577 | 0.0002577 |
| SIRPD | −0.9825 | 8.69E−06 | 8.69E−06 |
| PRAM1 | −0.9831 | 3.00E−08 | 3.00E−08 |
| LOC101928841 | −0.9836 | 0.0008729 | 0.0008729 |
| CD101 | −0.9851 | 0.004417 | 0.004417 |
| IGSF9 | −0.9853 | 0.0008233 | 0.0008233 |
| SLC22A4 | −0.9885 | 4.78E−07 | 4.78E−07 |
| LOC102723894 | −0.9952 | 5.57E−05 | 5.57E−05 |
| MARCO | −0.997 | 0.000231 | 0.000231 |
| OR2A1-AS1 | −0.997 | 0.003193 | 0.003193 |
| EPHA1-AS1 | −0.998 | 0.001485 | 0.001485 |
| LRRK2 | −1.001 | 3.55E−06 | 3.55E−06 |
| SERHL2 | −1.003 | 0.0008259 | 0.0008259 |
| ACOX2 | −1.008 | 0.00447 | 0.00447 |
| IL2RB | −1.011 | 8.34E−05 | 8.34E−05 |
| TMEM229B | −1.014 | 6.86E−11 | 6.86E−11 |
| ASB2 | −1.021 | 1.33E−05 | 1.33E−05 |
| SEMA3A | −1.021 | 4.63E−05 | 4.63E−05 |
| PTGER3 | −1.021 | 0.0002156 | 0.0002156 |
| FCAMR | −1.024 | 0.009561 | 0.009561 |
| CD36 | −1.033 | 1.32E−12 | 1.32E−12 |
| LOC101929174 | −1.039 | 0.0001858 | 0.0001858 |
| SIGLEC12 | −1.04 | 6.53E−09 | 6.53E−09 |
| VIT | −1.043 | 7.78E−08 | 7.78E−08 |
| EGR1 | −1.043 | 1.02E−05 | 1.02E−05 |
| PADI2 | −1.045 | 1.02E−07 | 1.02E−07 |
| PTPN22 | −1.046 | 1.92E−10 | 1.92E−10 |
| LPAR4 | −1.047 | 8.88E−08 | 8.88E−08 |
| MOXD1 | −1.048 | 1.08E−08 | 1.08E−08 |
| SLCO4C1 | −1.071 | 6.55E−08 | 6.55E−08 |
| FRMD3 | −1.074 | 3.88E−09 | 3.88E−09 |
| SYNPO | −1.079 | 1.87E−06 | 1.87E−06 |
| CSGALNACT1 | −1.079 | 5.05E−06 | 5.05E−06 |
| IL3RA | −1.081 | 3.49E−09 | 3.49E−09 |
| SPIB | −1.088 | 6.80E−07 | 6.80E−07 |
| C1orf127 | −1.091 | 6.99E−06 | 6.99E−06 |
| DDR2 | −1.094 | 9.10E−05 | 9.10E−05 |
| LAMA2 | −1.103 | 5.24E−05 | 5.24E−05 |
| TLR6 | −1.109 | 3.26E−08 | 3.26E−08 |
| LOC102724661 | −1.11 | 0.001586 | 0.001586 |
| IL8 | −1.117 | 0.0001554 | 0.0001554 |
| CLSTN2 | −1.129 | 6.27E−13 | 6.27E−13 |
| C1S | −1.133 | 3.71E−06 | 3.71E−06 |
| CIITA | −1.143 | 0.0001472 | 0.0001472 |
| PTCRA | −1.143 | 0.00412 | 0.00412 |
| EPHA1 | −1.149 | 5.60E−06 | 5.60E−06 |
| HRC | −1.149 | 0.001155 | 0.001155 |
| HAL | −1.151 | 4.33E−12 | 4.33E−12 |
| FYB | −1.152 | 1.37E−11 | 1.37E−11 |
| SH2D3C | −1.159 | 1.71E−07 | 1.71E−07 |
| GPR133 | −1.171 | 2.23E−05 | 2.23E−05 |
| C10orf91 | −1.173 | 3.27E−13 | 3.27E−13 |
| SOCS1 | −1.196 | 0.0004241 | 0.0004241 |
| GABBR2 | −1.215 | 6.23E−07 | 6.23E−07 |
| CMYA5 | −1.22 | 2.55E−10 | 2.55E−10 |
| FCGR1B | −1.221 | 2.23E−11 | 2.23E−11 |
| METTL7B | −1.224 | 5.08E−15 | 5.08E−15 |
| ANXA8 | −1.224 | 7.86E−09 | 7.86E−09 |
| CUX2 | −1.225 | 3.81E−07 | 3.81E−07 |
| PNPLA1 | −1.233 | 3.22E−06 | 3.22E−06 |
| ANKRD55 | −1.27 | 2.06E−07 | 2.06E−07 |
| MYOZ1 | −1.292 | 1.69E−07 | 1.69E−07 |
| LINC00968 | −1.305 | 0.0004354 | 0.0004354 |
| RGS18 | −1.313 | 6.84E−12 | 6.84E−12 |
| NFE2 | −1.357 | 6.54E−13 | 6.54E−13 |
| SH3TC2 | −1.362 | 3.10E−06 | 3.10E−06 |
| NKG7 | −1.379 | 4.76E−08 | 4.76E−08 |
| FBXO15 | −1.382 | 2.14E−05 | 2.14E−05 |
| CSPG4 | −1.396 | 8.53E−10 | 8.53E−10 |
| SHISA8 | −1.407 | 0.0001636 | 0.0001636 |
| DUSP27 | −1.409 | 9.77E−09 | 9.77E−09 |
| PRLR | −1.416 | 3.02E−12 | 3.02E−12 |
| RP11-330A16.1 | −1.416 | 9.49E−06 | 9.49E−06 |
| IGSF6 | −1.417 | 5.53E−10 | 5.53E−10 |
| C10orf10 | −1.438 | 6.15E−06 | 6.15E−06 |
| GPR141 | −1.441 | 9.59E−07 | 9.59E−07 |
| SEMG1 | −1.446 | 5.26E−06 | 5.26E−06 |
| HSD11B1 | −1.447 | 8.43E−13 | 8.43E−13 |
| ULK4P1 | −1.452 | 0.006578 | 0.006578 |
| LGALS12 | −1.455 | 1.31E−08 | 1.31E−08 |
| CLIC5 | −1.459 | 2.70E−05 | 2.70E−05 |
| IL31RA | −1.47 | 2.95E−08 | 2.95E−08 |
| ANXA8L1 | −1.473 | 5.86E−06 | 5.86E−06 |
| SIGLEC6 | −1.489 | 2.59E−10 | 2.59E−10 |
| KCNF1 | −1.511 | 6.58E−06 | 6.58E−06 |
| CD180 | −1.512 | 7.64E−11 | 7.64E−11 |
| KMO | −1.531 | 2.92E−08 | 2.92E−08 |
| TGM5 | −1.532 | 7.96E−10 | 7.96E−10 |
| LOC101927934 | −1.533 | 7.89E−05 | 7.89E−05 |
| LOC100130698 | −1.545 | 2.56E−05 | 2.56E−05 |
| HMCN1 | −1.57 | 1.68E−05 | 1.68E−05 |
| CAMK4 | −1.571 | 5.12E−06 | 5.12E−06 |
| FAM135B | −1.606 | 1.91E−09 | 1.91E−09 |
| ROS1 | −1.612 | 3.49E−10 | 3.49E−10 |
| KCNS1 | −1.651 | 9.49E−07 | 9.49E−07 |
| TGM4 | −1.665 | 7.08E−08 | 7.08E−08 |
| LOC101928109 | −1.668 | 8.08E−07 | 8.08E−07 |
| DLGAP1-AS3 | −1.678 | 1.76E−05 | 1.76E−05 |
| NRG4 | −1.74 | 9.85E−08 | 9.85E−08 |
| RNU2-2P | −1.742 | 0.03418 | 0.03418 |
| LOC101926956 | −1.765 | 5.41E−07 | 5.41E−07 |
| HHLA2 | −1.793 | 3.22E−08 | 3.22E−08 |
| GRIN3A | −2.04 | 2.09E−06 | 2.09E−06 |
| LINC00211 | −2.08 | 3.90E−08 | 3.90E−08 |
| HP | −2.224 | 3.92E−08 | 3.92E−08 |

TABLE 4
Differentially expressed genes with
IRAK4 degrader vs DMSO in THP 1 cells

| Gene | Log(FC) | P Value | Adj P Value |
|---|---|---|---|
| DGCR6 | 3.809 | 0.009345 | 0.009345 |
| PPFIA2 | 2.06 | 2.01E−05 | 2.01E−05 |
| OSBPL10 | 2.025 | 1.18E−05 | 1.18E−05 |
| ADAM12 | 1.84 | 0.0001329 | 0.0001329 |
| TREML1 | 1.81 | 8.05E−07 | 8.05E−07 |
| OSMR | 1.809 | 4.07E−05 | 4.07E−05 |
| GPX8 | 1.746 | 1.41E−08 | 1.41E−08 |
| FAM157C | 1.726 | 0.005339 | 0.005339 |
| PDGFRL | 1.679 | 0.001351 | 0.001351 |
| SYDE1 | 1.594 | 9.01E−05 | 9.01E−05 |
| ACOXL | 1.573 | 0.0002067 | 0.0002067 |
| CLEC10A | 1.547 | 6.72E−06 | 6.72E−06 |
| ST7-AS1 | 1.53 | 1.36E−05 | 1.36E−05 |
| CTSK | 1.499 | 1.43E−08 | 1.43E−08 |
| ZFHX4 | 1.419 | 9.13E−05 | 9.13E−05 |
| IL17RE | 1.412 | 1.15E−06 | 1.15E−06 |
| VSIG4 | 1.395 | 0.0001535 | 0.0001535 |
| SLAMF8 | 1.389 | 1.68E−05 | 1.68E−05 |
| MEOX2 | 1.385 | 2.84E−05 | 2.84E−05 |
| KIAA1324 | 1.342 | 1.44E−05 | 1.44E−05 |
| PARVA | 1.34 | 6.95E−10 | 6.95E−10 |
| PDE11A | 1.234 | 0.0006406 | 0.0006406 |
| TGFB1I1 | 1.225 | 6.10E−06 | 6.10E−06 |
| EPB41L1 | 1.221 | 1.32E−05 | 1.32E−05 |
| SCN8A | 1.211 | 0.002232 | 0.002232 |
| TEAD2 | 1.209 | 0.0001149 | 0.0001149 |
| DCST2 | 1.209 | 0.0005518 | 0.0005518 |
| EMP2 | 1.206 | 6.21E−07 | 6.21E−07 |
| ARHGAP31 | 1.186 | 8.84E−07 | 8.84E−07 |
| NRP2 | 1.182 | 1.10E−05 | 1.10E−05 |
| LOC100129083 | 1.179 | 0.000173 | 0.000173 |
| CGN | 1.172 | 0.001115 | 0.001115 |
| COL7A1 | 1.17 | 0.00351 | 0.00351 |
| C1orf51 | 1.168 | 0.0004074 | 0.0004074 |
| ACE | 1.151 | 1.92E−06 | 1.92E−06 |
| LOC101927119 | 1.145 | 0.0008609 | 0.0008609 |
| TNXB | 1.131 | 7.70E−05 | 7.70E−05 |
| LOC101928038 | 1.128 | 0.001865 | 0.001865 |
| ADORA1 | 1.124 | 0.003859 | 0.003859 |
| ELFN2 | 1.12 | 0.002394 | 0.002394 |
| CNRIP1 | 1.112 | 5.37E−06 | 5.37E−06 |
| PALLD | 1.109 | 7.19E−06 | 7.19E−06 |
| FLJ45513 | 1.095 | 0.001537 | 0.001537 |
| SIGLEC11 | 1.081 | 9.79E−06 | 9.79E−06 |
| ABCC3 | 1.075 | 0.000747 | 0.000747 |
| ITIH4 | 1.07 | 0.0003538 | 0.0003538 |
| CTRL | 1.065 | 0.0002332 | 0.0002332 |
| FAM57B | 1.057 | 2.83E−05 | 2.83E−05 |
| TMEM145 | 1.053 | 0.0005918 | 0.0005918 |
| ZNF703 | 1.039 | 3.04E−06 | 3.04E−06 |
| CXCL10 | 1.039 | 0.004186 | 0.004186 |
| CD6 | 1.035 | 0.0003271 | 0.0003271 |
| ETV7 | 1.033 | 0.001047 | 0.001047 |
| ESAM | 1.029 | 0.0005568 | 0.0005568 |
| FMN1 | 1.023 | 0.0003309 | 0.0003309 |
| DRP2 | 1.021 | 0.0004556 | 0.0004556 |
| WLS | 1.018 | 6.19E−07 | 6.19E−07 |
| TREM2 | 1.015 | 5.53E−08 | 5.53E−08 |
| LOC101927759 | 1.008 | 0.001649 | 0.001649 |
| TMEM132A | 1.007 | 0.001164 | 0.001164 |
| MTMR9LP | 1.005 | 0.002096 | 0.002096 |
| PRTFDC1 | 0.9999 | 5.97E−05 | 5.97E−05 |
| CYP19A1 | 0.9982 | 6.15E−05 | 6.15E−05 |
| TENC1 | 0.9976 | 1.25E−05 | 1.25E−05 |
| FBXO39 | 0.9949 | 0.004449 | 0.004449 |
| SLC23A3 | 0.9786 | 0.01089 | 0.01089 |
| DND1 | 0.9739 | 0.006522 | 0.006522 |
| CLEC5A | 0.9722 | 0.0007795 | 0.0007795 |
| RCN3 | 0.9655 | 0.0002108 | 0.0002108 |
| SIGLEC1 | 0.9624 | 3.08E−08 | 3.08E−08 |
| DPF3 | 0.9551 | 1.30E−05 | 1.30E−05 |
| C3AR1 | 0.9486 | 1.06E−05 | 1.06E−05 |
| PCGF2 | 0.9426 | 4.35E−06 | 4.35E−06 |
| SH3D19 | 0.9398 | 5.51E−06 | 5.51E−06 |
| KIFC3 | 0.9383 | 3.01E−08 | 3.01E−08 |
| LOC728715 | 0.9371 | 0.006749 | 0.006749 |
| DLGAP1-AS3 | 0.9343 | 0.003231 | 0.003231 |
| PDPN | 0.934 | 0.0135 | 0.0135 |
| SPTBN5 | 0.9333 | 0.0004318 | 0.0004318 |
| LOC100507424 | 0.9213 | 0.03029 | 0.03029 |
| TP53I3 | 0.9165 | 0.0003618 | 0.0003618 |
| STON2 | 0.9074 | 0.0002017 | 0.0002017 |
| ACY3 | 0.9004 | 0.00569 | 0.00569 |
| WNK4 | 0.8958 | 0.001176 | 0.001176 |
| LOC100130705 | 0.8931 | 0.01584 | 0.01584 |
| LINC01140 | 0.8923 | 0.0001283 | 0.0001283 |
| COL12A1 | 0.8907 | 0.000228 | 0.000228 |
| FRMD4A | 0.8857 | 3.35E−06 | 3.35E−06 |
| ASIC3 | 0.8853 | 0.008545 | 0.008545 |
| NUP62CL | 0.8845 | 0.0033 | 0.0033 |
| RAB13 | 0.8838 | 7.87E−09 | 7.87E−09 |
| GPR68 | 0.8786 | 0.01129 | 0.01129 |
| DNM1P35 | 0.8768 | 0.00413 | 0.00413 |
| EPHB1 | 0.8762 | 4.92E−08 | 4.92E−08 |
| HFE | 0.8753 | 0.006761 | 0.006761 |
| CNKSR1 | 0.8752 | 0.0262 | 0.0262 |
| SLC25A34 | 0.8676 | 0.0008293 | 0.0008293 |
| OAS3 | 0.8644 | 5.14E−08 | 5.14E−08 |
| CEACAM19 | 0.8599 | 0.0001513 | 0.0001513 |
| C1QC | 0.8531 | 0.03751 | 0.03751 |
| GPR141 | 0.8504 | 0.0001626 | 0.0001626 |
| ZDHHC11 | 0.8496 | 0.001132 | 0.001132 |
| MIR1304 | 0.848 | 0.01285 | 0.01285 |
| MAEL | 0.8478 | 0.01712 | 0.01712 |
| WDFY3-AS2 | 0.8469 | 0.002496 | 0.002496 |
| TMEM86A | 0.8452 | 0.001029 | 0.001029 |
| SLC17A7 | 0.8444 | 0.0004026 | 0.0004026 |
| PPP1R13L | 0.8416 | 7.58E−05 | 7.58E−05 |
| CMTM1 | 0.8404 | 0.0003617 | 0.0003617 |
| KCP | 0.8372 | 0.0006037 | 0.0006037 |
| UCP3 | 0.8346 | 0.0006094 | 0.0006094 |
| NHS | 0.8343 | 0.02638 | 0.02638 |
| CCDC168 | 0.833 | 0.0248 | 0.0248 |
| C15orf62 | 0.832 | 0.01743 | 0.01743 |
| CCDC103 | 0.8305 | 0.000111 | 0.000111 |
| SAMD14 | 0.8275 | 0.001003 | 0.001003 |
| TLX1 | 0.8267 | 0.0004001 | 0.0004001 |
| RASA4B | 0.8256 | 0.002978 | 0.002978 |
| PIP5KL1 | 0.8222 | 0.0001107 | 0.0001107 |
| TNFAIP8L3 | 0.8214 | 0.001828 | 0.001828 |
| STXBP5-AS1 | 0.8147 | 0.004513 | 0.004513 |
| TMEM9B-AS1 | 0.8128 | 0.003187 | 0.003187 |
| AP000330.8 | 0.8121 | 0.007586 | 0.007586 |
| TNF | 0.8082 | 0.0002992 | 0.0002992 |
| SULT1A2 | 0.8036 | 0.04001 | 0.04001 |
| ACSS3 | 0.8011 | 5.43E−06 | 5.43E−06 |
| MYOZ1 | 0.7996 | 9.89E−06 | 9.89E−06 |
| MAFB | 0.7995 | 1.19E−05 | 1.19E−05 |
| ZNF462 | 0.7988 | 0.002408 | 0.002408 |
| LOC100506083 | 0.7987 | 0.009823 | 0.009823 |
| EMR4P | 0.792 | 0.001349 | 0.001349 |
| GNG12 | 0.7908 | 0.000786 | 0.000786 |
| SLC6A11 | 0.7889 | 0.0005212 | 0.0005212 |
| LINC01094 | 0.7883 | 0.001385 | 0.001385 |
| ASIC1 | 0.7864 | 1.68E−06 | 1.68E−06 |
| ST6GALNAC2 | 0.7857 | 0.001085 | 0.001085 |
| SAP25 | 0.7837 | 0.003548 | 0.003548 |
| ERVMER34-1 | 0.783 | 0.000567 | 0.000567 |
| SIGLEC16 | 0.7817 | 0.001478 | 0.001478 |
| ALDH7A1 | 0.7814 | 0.0004313 | 0.0004313 |
| SLC7A5P2 | 0.7784 | 0.007366 | 0.007366 |
| FOXF1 | 0.7783 | 0.0005357 | 0.0005357 |
| FSTL1 | 0.7768 | 7.52E−06 | 7.52E−06 |
| RDM1 | 0.7755 | 0.002135 | 0.002135 |
| ITGA7 | 0.7751 | 0.0002095 | 0.0002095 |
| NXPH4 | 0.7731 | 0.01052 | 0.01052 |
| BZRAP1 | 0.77 | 0.002838 | 0.002838 |
| AASS | 0.7689 | 0.001218 | 0.001218 |
| TTC9 | 0.7631 | 0.01821 | 0.01821 |
| EFCAB4A | 0.7605 | 0.0007022 | 0.0007022 |
| CBWD3 | 0.7604 | 0.03521 | 0.03521 |
| DUSP27 | 0.7571 | 3.80E−05 | 3.80E−05 |
| MDGA1 | 0.7551 | 0.0009009 | 0.0009009 |
| NGFR | 0.7531 | 0.009821 | 0.009821 |
| HSPA7 | 0.7529 | 0.01421 | 0.01421 |
| CCDC154 | 0.7503 | 0.01697 | 0.01697 |
| IPO9-AS1 | 0.7483 | 0.04274 | 0.04274 |
| UCN | 0.748 | 0.003772 | 0.003772 |
| SLC7A7 | 0.7473 | 7.00E−07 | 7.00E−07 |
| ZNF692 | 0.747 | 2.79E−06 | 2.79E−06 |
| PTPN14 | 0.7468 | 1.16E−05 | 1.16E−05 |
| PKD1L3 | 0.7459 | 0.003647 | 0.003647 |
| IL17RC | 0.7449 | 0.0003498 | 0.0003498 |
| RASGRP3 | 0.7449 | 0.003997 | 0.003997 |
| ADAM19 | 0.7416 | 0.01334 | 0.01334 |
| PRR22 | 0.7408 | 0.001105 | 0.001105 |
| TTLL3 | 0.7397 | 0.006413 | 0.006413 |
| TMEM255B | 0.7394 | 0.002404 | 0.002404 |
| PYROXD2 | 0.7385 | 6.06E−05 | 6.06E−05 |
| GNMT | 0.7369 | 0.0343 | 0.0343 |
| CSPG4 | 0.7368 | 5.49E−06 | 5.49E−06 |
| ST5 | 0.7363 | 0.008974 | 0.008974 |
| ATP8B1 | 0.7357 | 3.01E−05 | 3.01E−05 |
| CCDC183 | 0.7354 | 0.001773 | 0.001773 |
| GJC2 | 0.7345 | 0.01838 | 0.01838 |
| LOC101928055 | 0.7325 | 0.006065 | 0.006065 |
| LINC00663 | 0.7322 | 0.03076 | 0.03076 |
| ADAMTS15 | 0.7281 | 4.84E−06 | 4.84E−06 |
| LOC101929112 | 0.7273 | 0.004874 | 0.004874 |
| PLA2G4C | 0.7272 | 0.0001224 | 0.0001224 |
| GAS6-AS2 | 0.7271 | 0.0004976 | 0.0004976 |
| EPHA1 | 0.7244 | 0.0002962 | 0.0002962 |
| IGFBP4 | 0.723 | 1.97E−07 | 1.97E−07 |
| SPHK1 | 0.7228 | 0.0001541 | 0.0001541 |
| SEC31B | 0.7222 | 0.00177 | 0.00177 |
| ADAMTS17 | 0.7209 | 0.0007844 | 0.0007844 |
| SEMA6B | 0.72 | 3.49E−05 | 3.49E−05 |
| DOK4 | 0.7192 | 0.0001361 | 0.0001361 |
| YJEFN3 | 0.7187 | 0.0008396 | 0.0008396 |
| BIN1 | 0.7172 | 3.85E−06 | 3.85E−06 |
| EFCAB12 | 0.717 | 0.007817 | 0.007817 |
| SEMA3D | 0.716 | 0.01923 | 0.01923 |
| SCML1 | 0.711 | 0.01977 | 0.01977 |
| CLDN7 | 0.7103 | 0.002722 | 0.002722 |
| RP11-15A1.7 | 0.7096 | 0.01305 | 0.01305 |
| NPAS4 | 0.7093 | 0.0151 | 0.0151 |
| RAI14 | 0.7085 | 3.27E−07 | 3.27E−07 |
| AMOTL2 | 0.7081 | 7.21E−05 | 7.21E−05 |
| MOB3B | 0.7081 | 0.00972 | 0.00972 |
| ZNF491 | 0.7057 | 0.01952 | 0.01952 |
| RP5-1180C10.2 | 0.7048 | 0.001957 | 0.001957 |
| FAM83B | 0.7036 | 0.0006643 | 0.0006643 |
| SGK1 | 0.7027 | 0.001916 | 0.001916 |
| ZFHX2 | 0.7007 | 0.002393 | 0.002393 |
| C1RL-AS1 | 0.7006 | 0.003501 | 0.003501 |
| SNTB2 | 0.7004 | 0.0002471 | 0.0002471 |
| RPS18P9 | 0.6993 | 0.008565 | 0.008565 |
| ZNF487 | 0.6987 | 0.01634 | 0.01634 |
| SERPINH1 | 0.696 | 3.85E−07 | 3.85E−07 |
| ARMCX5-GPRASP2 | 0.6881 | 0.01107 | 0.01107 |
| GSTM2 | 0.6815 | 0.03953 | 0.03953 |
| TEAD3 | 0.6802 | 5.29E−05 | 5.29E−05 |
| LTB4R2 | 0.6791 | 0.0003389 | 0.0003389 |
| KLRAP1 | 0.6778 | 0.03904 | 0.03904 |
| MIR570 | 0.6767 | 0.03771 | 0.03771 |
| LRRC4B | 0.6766 | 6.89E−05 | 6.89E−05 |
| MANEA-AS1 | 0.6759 | 0.03958 | 0.03958 |
| RP11-307C12.11 | 0.675 | 0.001382 | 0.001382 |
| ARAP3 | 0.6734 | 0.0001437 | 0.0001437 |
| PXDC1 | 0.6721 | 0.003061 | 0.003061 |
| TBC1D9 | 0.6706 | 5.22E−05 | 5.22E−05 |
| DNHD1 | 0.667 | 0.0003086 | 0.0003086 |
| CCDC155 | 0.6658 | 0.0269 | 0.0269 |
| PLEKHA4 | 0.6652 | 0.0001923 | 0.0001923 |
| STXBP6 | 0.6645 | 8.24E−05 | 8.24E−05 |
| CCDC27 | 0.6611 | 0.002215 | 0.002215 |
| ITGB2-AS1 | 0.6608 | 0.02077 | 0.02077 |
| LINC00337 | 0.6601 | 0.03059 | 0.03059 |
| MROH6 | 0.6588 | 0.00252 | 0.00252 |
| CALHM2 | 0.6574 | 9.82E−05 | 9.82E−05 |
| QRICH2 | 0.6567 | 0.007838 | 0.007838 |
| IFIT3 | 0.6563 | 6.47E−06 | 6.47E−06 |
| SEMA3B | 0.6545 | 0.03474 | 0.03474 |
| BLNK | 0.654 | 0.002566 | 0.002566 |
| HGD | 0.6528 | 0.0005552 | 0.0005552 |
| RP3-399L15.3 | 0.6516 | 0.0001035 | 0.0001035 |
| REC8 | 0.651 | 0.007974 | 0.007974 |
| MGC16142 | 0.6505 | 0.04274 | 0.04274 |
| FBXL8 | 0.6504 | 0.001621 | 0.001621 |
| ATP2B3 | 0.6497 | 0.006879 | 0.006879 |
| RND3 | 0.6494 | 0.002165 | 0.002165 |
| LOC100505771 | 0.6492 | 0.01974 | 0.01974 |
| NRP1 | 0.6479 | 0.0001242 | 0.0001242 |
| PIPOX | 0.6465 | 4.46E−05 | 4.46E−05 |
| ADCY4 | 0.644 | 0.03089 | 0.03089 |
| BTN2A3P | 0.6432 | 0.005706 | 0.005706 |
| USH1C | 0.6382 | 0.003174 | 0.003174 |
| MLXIPL | 0.6377 | 0.007901 | 0.007901 |
| CLK1 | 0.6367 | 2.06E−06 | 2.06E−06 |
| NR1H3 | 0.6367 | 1.26E−05 | 1.26E−05 |
| KCNJ5 | 0.6358 | 0.007752 | 0.007752 |
| VAX2 | 0.6346 | 0.006492 | 0.006492 |
| CD1C | 0.6342 | 0.02268 | 0.02268 |
| AC007038.7 | 0.6314 | 0.01056 | 0.01056 |
| DHRS3 | 0.6307 | 0.005189 | 0.005189 |
| DAB2 | 0.6294 | 2.10E−05 | 2.10E−05 |
| TBX15 | 0.6293 | 0.001029 | 0.001029 |
| PRSS53 | 0.6281 | 0.001429 | 0.001429 |
| DUSP4 | 0.6271 | 0.0002337 | 0.0002337 |
| CUEDC1 | 0.6243 | 2.22E−05 | 2.22E−05 |
| ASMTL-AS1 | 0.6242 | 0.01732 | 0.01732 |
| QPCT | 0.624 | 0.00212 | 0.00212 |
| TTLL7 | 0.623 | 0.01612 | 0.01612 |
| LOC101927321 | 0.6228 | 0.02817 | 0.02817 |
| CEACAM4 | 0.6227 | 0.00696 | 0.00696 |
| FFAR4 | 0.6221 | 0.00907 | 0.00907 |
| NUPR1 | 0.6201 | 0.01145 | 0.01145 |
| TTC23 | 0.6179 | 4.07E−06 | 4.07E−06 |
| DPYD | 0.6177 | 1.26E−07 | 1.26E−07 |
| EGR3 | 0.6175 | 0.01362 | 0.01362 |
| CROCCP3 | 0.6154 | 0.004248 | 0.004248 |
| ADAMTS4 | 0.6146 | 0.003386 | 0.003386 |
| GPR84 | 0.6139 | 0.002879 | 0.002879 |
| NEAT1 | 0.6129 | 0.004838 | 0.004838 |
| LOC101926956 | 0.6127 | 0.006163 | 0.006163 |
| AURKC | 0.6123 | 0.01388 | 0.01388 |
| GPR113 | 0.6105 | 0.02041 | 0.02041 |
| LOC100506548 | 0.6092 | 1.91E−06 | 1.91E−06 |
| GDPD3 | 0.6082 | 0.01922 | 0.01922 |
| SIPA1L1 | 0.6081 | 3.97E−05 | 3.97E−05 |
| PPP1R1C | 0.6074 | 0.01131 | 0.01131 |
| LAX1 | 0.6066 | 0.01031 | 0.01031 |
| TLR5 | 0.6066 | 0.04147 | 0.04147 |
| PCDHGA12 | 0.6055 | 0.005677 | 0.005677 |
| EPSTI1 | 0.6007 | 0.0001589 | 0.0001589 |
| CACTIN-AS1 | 0.5982 | 0.01303 | 0.01303 |
| ANK1 | 0.5978 | 0.01365 | 0.01365 |
| RASGRF1 | 0.5973 | 0.002775 | 0.002775 |
| VAMP5 | 0.5964 | 0.04627 | 0.04627 |
| LINC00877 | 0.5956 | 0.03995 | 0.03995 |
| CBX6 | 0.5943 | 4.61E−07 | 4.61E−07 |
| BACE1 | 0.5942 | 6.12E−08 | 6.12E−08 |
| CTGF | 0.591 | 0.00699 | 0.00699 |
| ZNF665 | 0.5908 | 0.03949 | 0.03949 |
| PAQR6 | 0.5901 | 0.003754 | 0.003754 |
| TTC21A | 0.5886 | 0.02085 | 0.02085 |
| SPTB | 0.5877 | 0.008665 | 0.008665 |
| CD14 | 0.5874 | 0.01658 | 0.01658 |
| LOC102724217 | 0.5853 | 0.007105 | 0.007105 |
| CIITA | −0.5914 | 0.03258 | 0.03258 |
| EPHX4 | −0.5929 | 0.01281 | 0.01281 |
| NLRC3 | −0.5939 | 0.001443 | 0.001443 |
| SLC7A2 | −0.5943 | 0.001 | 0.001 |
| CXCR4 | −0.5961 | 2.57E−07 | 2.57E−07 |
| CLUHP3 | −0.5966 | 0.04581 | 0.04581 |
| SHISA7 | −0.6063 | 0.0003919 | 0.0003919 |
| PXK | −0.6096 | 1.04E−07 | 1.04E−07 |
| CAV1 | −0.6103 | 0.0005454 | 0.0005454 |
| PRG2 | −0.6148 | 0.007062 | 0.007062 |
| HGF | −0.6155 | 0.0007808 | 0.0007808 |
| KCNA3 | −0.617 | 6.54E−05 | 6.54E−05 |
| NRG4 | −0.619 | 0.005312 | 0.005312 |
| ANKRD55 | −0.6194 | 0.0007287 | 0.0007287 |
| DBNDD1 | −0.6216 | 0.008926 | 0.008926 |
| SLA2 | −0.6218 | 0.007374 | 0.007374 |
| TRPV3 | −0.6266 | 0.04907 | 0.04907 |
| ST6GALNAC3 | −0.6268 | 0.007342 | 0.007342 |
| EMR1 | −0.6277 | 0.008108 | 0.008108 |
| FAM69B | −0.6279 | 5.15E−06 | 5.15E−06 |
| C17orf64 | −0.635 | 0.03323 | 0.03323 |
| CD84 | −0.6436 | 2.22E−07 | 2.22E−07 |
| SEMA3A | −0.6449 | 0.00526 | 0.00526 |
| MPZL1 | −0.6451 | 5.41E−05 | 5.41E−05 |
| IL20RB | −0.6457 | 0.01414 | 0.01414 |
| SYNPO2 | −0.6472 | 2.42E−06 | 2.42E−06 |
| TMEM150A | −0.6474 | 0.008799 | 0.008799 |
| VASN | −0.6475 | 0.007328 | 0.007328 |
| ZNF891 | −0.6524 | 0.01175 | 0.01175 |
| ZMYND10 | −0.6531 | 0.02976 | 0.02976 |
| MLC1 | −0.6577 | 3.48E−07 | 3.48E−07 |
| CUX2 | −0.6584 | 0.00311 | 0.00311 |
| LOC728819 | −0.6633 | 0.01192 | 0.01192 |
| PTAFR | −0.6643 | 2.27E−05 | 2.27E−05 |
| GPM6B | −0.6692 | 0.004281 | 0.004281 |
| GOLGA8EP | −0.6799 | 0.04995 | 0.04995 |
| CCNB3 | −0.6872 | 0.02893 | 0.02893 |
| PLXNA4 | −0.6889 | 0.00329 | 0.00329 |
| ZCWPW2 | −0.6945 | 0.03201 | 0.03201 |
| FAM46B | −0.6967 | 0.004989 | 0.004989 |
| SORBS1 | −0.7035 | 0.01427 | 0.01427 |
| FCER2 | −0.7125 | 3.21E−05 | 3.21E−05 |
| NKD2 | −0.7164 | 1.85E−06 | 1.85E−06 |
| TMEM176B | −0.7176 | 0.0005188 | 0.0005188 |
| NPAS1 | −0.718 | 0.002538 | 0.002538 |
| AKAP7 | −0.719 | 1.76E−05 | 1.76E−05 |
| TERT | −0.7203 | 0.0002245 | 0.0002245 |
| CORO2A | −0.7299 | 5.36E−06 | 5.36E−06 |
| EBF3 | −0.7345 | 4.93E−06 | 4.93E−06 |
| CST7 | −0.7412 | 0.001926 | 0.001926 |
| PLAC8 | −0.7588 | 1.60E−08 | 1.60E−08 |
| C6orf223 | −0.7674 | 2.49E−05 | 2.49E−05 |
| HSH2D | −0.7767 | 2.13E−06 | 2.13E−06 |
| PPP1R27 | −0.7803 | 1.52E−06 | 1.52E−06 |
| LOC102724005 | −0.7847 | 0.01254 | 0.01254 |
| SLC4A3 | −0.791 | 0.00801 | 0.00801 |
| SERPINB2 | −0.7957 | 6.09E−06 | 6.09E−06 |
| CHRFAM7A | −0.7978 | 0.03433 | 0.03433 |
| MRAP2 | −0.8016 | 0.0004844 | 0.0004844 |
| SAMD13 | −0.815 | 0.0007667 | 0.0007667 |
| IL31RA | −0.816 | 0.0003034 | 0.0003034 |
| MNX1-AS1 | −0.8198 | 0.005986 | 0.005986 |
| PMEPA1 | −0.8346 | 0.002697 | 0.002697 |
| AIF1L | −0.9276 | 0.006215 | 0.006215 |
| GPR158 | −0.9326 | 0.0001847 | 0.0001847 |
| LOC100996717 | −0.9425 | 0.0004041 | 0.0004041 |
| TNFAIP8L2-SCNM1 | −0.9742 | 0.008369 | 0.008369 |
| SEMG1 | −0.9769 | 0.001761 | 0.001761 |
| ARL4C | −1.01 | 0.0006149 | 0.0006149 |
| ELOVL3 | −1.016 | 8.56E−05 | 8.56E−05 |
| CIB3 | −1.023 | 0.004862 | 0.004862 |
| CSGALNACT1 | −1.159 | 4.84E−05 | 4.84E−05 |
| FAM157A | −1.186 | 0.0005428 | 0.0005428 |
| SEMA3C | −1.247 | 0.003467 | 0.003467 |

TABLE 5
Differentially expressed genes in IRAK4-KO vs WT THP1 cells

| Gene | Log(FC) | P Value | Adj P Value |
|---|---|---|---|
| ZBED6CL | 8.385 | 1.56E−11 | 8.78E−09 |
| PDE9A | 6.886 | 9.68E−09 | 3.25E−07 |
| BHLHE22 | 6.81 | 1.86E−14 | 8.43E−11 |
| SPATA31C1 | 6.337 | 1.95E−09 | 1.11E−07 |
| WIPF3 | 6.247 | 1.05E−07 | 1.77E−06 |
| GNLY | 5.453 | 5.89E−08 | 1.16E−06 |
| SHROOM2 | 5.282 | 1.76E−06 | 1.61E−05 |
| SSC5D | 5.271 | 1.53E−06 | 1.44E−05 |
| LINC00599 | 5.256 | 8.06E−06 | 5.28E−05 |
| TSPAN18 | 4.949 | 3.64E−07 | 4.74E−06 |
| C14orf132 | 4.829 | 8.18E−07 | 8.90E−06 |
| CYP27A1 | 4.818 | 1.51E−10 | 2.50E−08 |
| MOCS1 | 4.807 | 1.48E−06 | 1.40E−05 |
| LOC284240 | 4.776 | 2.35E−05 | 0.0001222 |
| SPP1 | 4.756 | 9.83E−13 | 1.67E−09 |
| DEFA4 | 4.719 | 1.92E−08 | 5.34E−07 |
| ZNF334 | 4.657 | 7.57E−08 | 1.38E−06 |
| TCEA3 | 4.6 | 4.40E−06 | 3.34E−05 |
| UNC5C | 4.51 | 4.42E−11 | 1.28E−08 |
| GP9 | 4.496 | 3.83E−08 | 8.55E−07 |
| KRT17 | 4.464 | 2.64E−07 | 3.65E−06 |
| LOC401463 | 4.429 | 3.58E−09 | 1.58E−07 |
| IGSF9 | 4.367 | 2.65E−11 | 1.13E−08 |
| ITGA1 | 4.343 | 2.75E−07 | 3.76E−06 |
| ERICH2 | 4.321 | 1.19E−05 | 7.20E−05 |
| AQP1 | 4.305 | 9.21E−11 | 1.96E−08 |
| TCEAL2 | 4.255 | 1.32E−05 | 7.77E−05 |
| CYP11A1 | 4.236 | 2.06E−08 | 5.63E−07 |
| MCTP2 | 4.235 | 1.64E−07 | 2.50E−06 |
| SLC22A17 | 4.159 | 3.98E−08 | 8.77E−07 |
| CYP1B1 | 4.122 | 6.94E−15 | 4.71E−11 |
| IGLON5 | 4.044 | 4.06E−07 | 5.13E−06 |
| SERPINB9 | 4.038 | 7.20E−06 | 4.86E−05 |
| CCL4 | 3.994 | 6.25E−08 | 1.21E−06 |
| SLAMF8 | 3.943 | 1.35E−09 | 9.05E−08 |
| BACH2 | 3.933 | 1.09E−06 | 1.11E−05 |
| PTGER2 | 3.901 | 4.81E−07 | 5.83E−06 |
| CST7 | 3.812 | 3.61E−10 | 3.86E−08 |
| ALDH1A2 | 3.767 | 1.07E−07 | 1.80E−06 |
| CLEC5A | 3.731 | 1.04E−09 | 7.81E−08 |
| LOC101927070 | 3.714 | 7.25E−09 | 2.63E−07 |
| AACSP1 | 3.697 | 2.34E−08 | 6.14E−07 |
| TMEM52B | 3.658 | 2.48E−06 | 2.11E−05 |
| PDPN | 3.607 | 1.23E−09 | 8.69E−08 |
| EPB41L3 | 3.604 | 8.08E−11 | 1.77E−08 |
| MGAM | 3.576 | 5.76E−08 | 1.14E−06 |
| PTCRA | 3.568 | 1.74E−09 | 1.03E−07 |
| CCL4L1 | 3.531 | 3.89E−07 | 4.97E−06 |
| SIGLEC7 | 3.483 | 1.53E−08 | 4.49E−07 |
| ZSWIM5 | 3.481 | 9.19E−08 | 1.60E−06 |
| SCN11A | 3.475 | 6.53E−08 | 1.24E−06 |
| POU4F2 | 3.462 | 1.21E−12 | 1.83E−09 |
| RHOH | 3.46 | 6.64E−07 | 7.51E−06 |
| ACSM5 | 3.452 | 2.98E−07 | 4.03E−06 |
| SLC2A10 | 3.438 | 2.41E−09 | 1.23E−07 |
| PRG2 | 3.404 | 5.60E−09 | 2.21E−07 |
| PTGDS | 3.393 | 5.06E−10 | 4.99E−08 |
| NRP2 | 3.372 | 2.07E−07 | 3.01E−06 |
| CTSW | 3.357 | 1.14E−09 | 8.27E−08 |
| FCAR | 3.312 | 2.25E−07 | 3.21E−06 |
| TREML2 | 3.299 | 2.48E−08 | 6.40E−07 |
| ZNF542 | 3.276 | 9.40E−07 | 9.98E−06 |
| LOC101928581 | 3.264 | 1.18E−08 | 3.74E−07 |
| CCDC120 | 3.241 | 5.13E−09 | 2.08E−07 |
| SOWAHC | 3.229 | 1.20E−08 | 3.79E−07 |
| FLT1 | 3.223 | 1.59E−05 | 8.97E−05 |
| NBEA | 3.21 | 1.40E−07 | 2.23E−06 |
| MFAP4 | 3.191 | 2.89E−07 | 3.92E−06 |
| SPOCD1 | 3.174 | 5.10E−06 | 3.75E−05 |
| KCNJ2 | 3.106 | 1.34E−06 | 1.30E−05 |
| RASGRF1 | 3.105 | 1.98E−08 | 5.47E−07 |
| VANGL2 | 3.086 | 2.58E−05 | 0.0001315 |
| CR1 | 3.081 | 3.72E−05 | 0.000177 |
| COL12A1 | 3.067 | 5.35E−08 | 1.09E−06 |
| EFCC1 | 3.062 | 7.69E−06 | 5.09E−05 |
| COL4A2 | 2.963 | 3.88E−09 | 1.69E−07 |
| PRKCDBP | 2.962 | 1.23E−07 | 2.00E−06 |
| ADRA2B | 2.956 | 9.92E−08 | 1.69E−06 |
| POPDC3 | 2.95 | 1.31E−07 | 2.11E−06 |
| RGS1 | 2.946 | 1.34E−05 | 7.84E−05 |
| CTSF | 2.93 | 5.42E−10 | 5.16E−08 |
| LOC102724908 | 2.92 | 2.90E−10 | 3.58E−08 |
| LOC102723741 | 2.92 | 3.72E−06 | 2.93E−05 |
| RGS20 | 2.916 | 5.61E−10 | 5.22E−08 |
| ITGAL | 2.915 | 2.63E−13 | 8.92E−10 |
| FPR2 | 2.905 | 2.81E−08 | 6.93E−07 |
| SYT17 | 2.882 | 2.56E−07 | 3.56E−06 |
| CDKL5 | 2.87 | 8.46E−09 | 2.95E−07 |
| GLIS3 | 2.861 | 6.18E−05 | 0.000266 |
| TRO | 2.823 | 9.69E−10 | 7.48E−08 |
| TMEM176B | 2.816 | 3.50E−09 | 1.57E−07 |
| KCNJ15 | 2.795 | 2.01E−06 | 1.78E−05 |
| FABP4 | 2.786 | 5.26E−06 | 3.84E−05 |
| ADAM19 | 2.777 | 2.84E−08 | 6.96E−07 |
| FAM163A | 2.762 | 9.91E−10 | 7.61E−08 |
| ANGPTL6 | 2.73 | 9.97E−07 | 1.04E−05 |
| MLLT4 | 2.729 | 1.45E−09 | 9.27E−08 |
| SVEP1 | 2.729 | 2.14E−08 | 5.75E−07 |
| NLRP2 | 2.725 | 1.94E−05 | 0.0001048 |
| TIAM2 | 2.707 | 3.31E−06 | 2.67E−05 |
| FAM46A | 2.698 | 5.13E−13 | 1.36E−09 |
| PDGFRL | 2.686 | 9.37E−06 | 5.94E−05 |
| LOC729737 | 2.681 | 9.50E−08 | 1.63E−06 |
| LINC00452 | 2.681 | 4.45E−07 | 5.48E−06 |
| FLJ36000 | 2.673 | 1.19E−08 | 3.76E−07 |
| SERPINA1 | 2.669 | 1.33E−08 | 4.09E−07 |
| CYP46A1 | 2.664 | 3.01E−08 | 7.23E−07 |
| LOC101927732 | 2.662 | 0.000136 | 0.0005043 |
| LOC101928173 | 2.643 | 9.64E−06 | 6.09E−05 |
| ALOX15 | 2.637 | 1.18E−06 | 1.18E−05 |
| SORT1 | 2.631 | 1.51E−11 | 8.78E−09 |
| MYO7B | 2.627 | 5.56E−08 | 1.12E−06 |
| ORM1 | 2.626 | 8.57E−10 | 6.95E−08 |
| NEO1 | 2.603 | 4.89E−11 | 1.30E−08 |
| IGFBP3 | 2.601 | 1.81E−08 | 5.11E−07 |
| AKAP12 | 2.596 | 1.06E−08 | 3.46E−07 |
| GSG1L | 2.595 | 1.48E−06 | 1.40E−05 |
| LOC101926956 | 2.583 | 1.19E−07 | 1.96E−06 |
| PLEKHN1 | 2.56 | 1.66E−06 | 1.54E−05 |
| NLRP1 | 2.558 | 4.01E−08 | 8.80E−07 |
| FN1 | 2.544 | 1.39E−06 | 1.35E−05 |
| ADAM12 | 2.543 | 4.44E−06 | 3.36E−05 |
| AOC1 | 2.515 | 3.48E−12 | 4.29E−09 |
| SNCAIP | 2.5 | 1.21E−05 | 7.27E−05 |
| ABCC3 | 2.498 | 1.93E−08 | 5.34E−07 |
| DMRT3 | 2.486 | 6.11E−08 | 1.19E−06 |
| DLG3 | 2.484 | 2.32E−06 | 1.99E−05 |
| TREM1 | 2.462 | 3.89E−08 | 8.62E−07 |
| MTSS1 | 2.454 | 1.30E−08 | 4.03E−07 |
| KIAA1211L | 2.438 | 2.93E−08 | 7.11E−07 |
| MGC57346 | 2.435 | 2.91E−06 | 2.42E−05 |
| MYO5C | 2.412 | 5.18E−07 | 6.19E−06 |
| TNFAIP8L3 | 2.41 | 1.39E−08 | 4.21E−07 |
| C8orf47 | 2.402 | 2.31E−06 | 1.99E−05 |
| DACT1 | 2.399 | 3.98E−05 | 0.0001869 |
| DYSF | 2.395 | 5.06E−06 | 3.73E−05 |
| SPRY4 | 2.377 | 2.62E−07 | 3.63E−06 |
| MGLL | 2.363 | 1.13E−08 | 3.61E−07 |
| LYG2 | 2.35 | 7.58E−05 | 0.0003143 |
| PEX5L | 2.347 | 1.87E−11 | 9.06E−09 |
| TGFB2 | 2.337 | 7.63E−07 | 8.42E−06 |
| SIT1 | 2.329 | 1.28E−05 | 7.56E−05 |
| IL8 | 2.31 | 7.69E−06 | 5.09E−05 |
| DTX3 | 2.291 | 1.60E−05 | 9.02E−05 |
| MEOX2 | 2.287 | 4.71E−07 | 5.72E−06 |
| TRIB2 | 2.281 | 1.27E−08 | 3.97E−07 |
| CCL2 | 2.281 | 8.55E−08 | 1.52E−06 |
| DDIT4L | 2.278 | 2.73E−07 | 3.75E−06 |
| ANTXR1 | 2.276 | 2.75E−10 | 3.46E−08 |
| ABI3 | 2.267 | 5.85E−05 | 0.0002543 |
| CCND1 | 2.26 | 5.35E−10 | 5.16E−08 |
| GPR27 | 2.25 | 4.98E−08 | 1.03E−06 |
| WWTR1 | 2.247 | 2.88E−08 | 6.99E−07 |
| AZU1 | 2.244 | 3.16E−11 | 1.13E−08 |
| PLCB4 | 2.235 | 6.94E−05 | 0.0002917 |
| GAS6-AS2 | 2.234 | 2.42E−10 | 3.22E−08 |
| SOGA3 | 2.227 | 1.68E−06 | 1.55E−05 |
| LPL | 2.226 | 7.00E−13 | 1.36E−09 |
| ABLIM3 | 2.217 | 9.68E−06 | 6.11E−05 |
| ROS1 | 2.215 | 9.71E−09 | 3.25E−07 |
| HOXC10 | 2.214 | 2.22E−06 | 1.93E−05 |
| LINC00565 | 2.213 | 4.22E−05 | 0.0001949 |
| SPOCK1 | 2.21 | 2.10E−08 | 5.69E−07 |
| ADAMTS15 | 2.202 | 1.01E−10 | 2.05E−08 |
| DLL4 | 2.199 | 6.81E−09 | 2.52E−07 |
| TM6SF1 | 2.198 | 3.79E−07 | 4.87E−06 |
| FAIM3 | 2.194 | 2.72E−05 | 0.000137 |
| CYP2J2 | 2.193 | 4.34E−05 | 0.0001995 |
| TJP3 | 2.191 | 8.30E−07 | 8.98E−06 |
| APOC1 | 2.188 | 2.30E−10 | 3.10E−08 |
| TUB | 2.177 | 3.68E−07 | 4.77E−06 |
| SCUBE2 | 2.176 | 0.000467 | 0.001396 |
| EPHA7 | 2.175 | 1.24E−05 | 7.36E−05 |
| SYT6 | 2.16 | 1.11E−06 | 1.13E−05 |
| NLRC3 | 2.154 | 7.88E−10 | 6.66E−08 |
| HP | 2.153 | 1.64E−08 | 4.74E−07 |
| TREML1 | 2.149 | 6.69E−09 | 2.48E−07 |
| TENM3 | 2.141 | 6.35E−08 | 1.22E−06 |
| KY | 2.131 | 1.54E−06 | 1.45E−05 |
| HMCN1 | 2.128 | 1.98E−07 | 2.92E−06 |
| CKAP4 | 2.126 | 6.20E−12 | 5.26E−09 |
| KIT | 2.126 | 8.14E−10 | 6.78E−08 |
| IGFBP2 | 2.124 | 2.89E−11 | 1.13E−08 |
| KIAA1147 | 2.122 | 8.67E−08 | 1.53E−06 |
| RP11-35612.4 | 2.122 | 7.76E−07 | 8.52E−06 |
| DYRK3 | 2.12 | 0.0001669 | 0.0005948 |
| CLSTN2 | 2.116 | 9.76E−09 | 3.26E−07 |
| CD101 | 2.102 | 7.88E−07 | 8.62E−06 |
| FBXO15 | 2.101 | 1.23E−06 | 1.22E−05 |
| NUAK2 | 2.101 | 0.0001873 | 0.0006528 |
| TMEM200A | 2.099 | 8.19E−06 | 5.35E−05 |
| C1orf186 | 2.098 | 3.22E−08 | 7.59E−07 |
| CPNE5 | 2.098 | 9.12E−06 | 5.82E−05 |
| VCAN | 2.096 | 6.25E−09 | 2.36E−07 |
| LOC100128374 | 2.095 | 1.07E−05 | 6.62E−05 |
| NMNAT2 | 2.084 | 4.38E−07 | 5.45E−06 |
| MFNG | 2.08 | 9.45E−12 | 7.13E−09 |
| PCOLCE2 | 2.08 | 5.94E−10 | 5.45E−08 |
| S100A12 | 2.079 | 0.0001443 | 0.0005299 |
| C17orf100 | 2.078 | 1.53E−05 | 8.74E−05 |
| ADAMTS6 | 2.078 | 7.80E−05 | 0.0003204 |
| CAPN6 | 2.076 | 2.99E−05 | 0.000148 |
| MMP2 | 2.073 | 3.95E−11 | 1.22E−08 |
| SLC44A2 | 2.069 | 5.32E−07 | 6.34E−06 |
| ALDH7A1 | 2.069 | 1.82E−06 | 1.66E−05 |
| ABHD4 | 2.067 | 1.94E−07 | 2.86E−06 |
| DSC3 | 2.065 | 2.60E−09 | 1.28E−07 |
| LSP1 | 2.062 | 4.95E−10 | 4.98E−08 |
| ACSS1 | 2.042 | 1.56E−10 | 2.50E−08 |
| ACP6 | 2.028 | 4.51E−08 | 9.63E−07 |
| ABCB4 | 2.028 | 4.41E−08 | 9.50E−07 |
| TCN2 | 2.017 | 6.79E−07 | 7.66E−06 |
| ALDH1A1 | 2.017 | 1.92E−06 | 1.72E−05 |
| CEACAM6 | 2.015 | 2.54E−10 | 3.31E−08 |
| A2M | 2.015 | 2.48E−09 | 1.24E−07 |
| GPR176 | 2.008 | 2.30E−05 | 0.0001202 |
| IL2RB | 2.006 | 6.82E−06 | 4.69E−05 |
| LOC100130093 | 2.001 | 6.54E−06 | 4.54E−05 |
| FAM135B | 1.999 | 4.62E−08 | 9.84E−07 |
| MAML2 | 1.997 | 2.30E−06 | 1.98E−05 |
| CLEC7A | 1.994 | 1.05E−05 | 6.50E−05 |
| TCF23 | 1.99 | 7.21E−08 | 1.34E−06 |
| ANPEP | 1.987 | 4.74E−11 | 1.30E−08 |
| ABCG1 | 1.987 | 2.09E−05 | 0.0001116 |
| MARCO | 1.985 | 1.79E−07 | 2.68E−06 |
| C3AR1 | 1.982 | 1.30E−08 | 4.03E−07 |
| MYH10 | 1.979 | 6.51E−09 | 2.43E−07 |
| ERVMER34-1 | 1.979 | 4.00E−07 | 5.08E−06 |
| PRR18 | 1.976 | 1.01E−05 | 6.33E−05 |
| IL12RB2 | 1.975 | 4.08E−10 | 4.30E−08 |
| PRDM13 | 1.971 | 1.11E−06 | 1.13E−05 |
| KIAA1244 | 1.961 | 6.84E−07 | 7.69E−06 |
| TFAP2C | 1.956 | 1.01E−05 | 6.32E−05 |
| TBC1D4 | 1.952 | 2.31E−07 | 3.29E−06 |
| PARK2 | 1.947 | 1.41E−05 | 8.19E−05 |
| S100A9 | 1.937 | 1.69E−10 | 2.64E−08 |
| APOE | 1.936 | 2.24E−09 | 1.17E−07 |
| EIF4E3 | 1.936 | 4.46E−08 | 9.59E−07 |
| AGT | 1.933 | 2.65E−11 | 1.13E−08 |
| FERMT2 | 1.929 | 1.77E−05 | 9.72E−05 |
| SIGLEC16 | 1.925 | 1.14E−07 | 1.88E−06 |
| ITGB5 | 1.922 | 1.37E−09 | 9.05E−08 |
| SLPI | 1.919 | 2.68E−10 | 3.44E−08 |
| CPXM1 | 1.915 | 2.16E−06 | 1.89E−05 |
| SEZ6L2 | 1.909 | 4.60E−11 | 1.30E−08 |
| NKG7 | 1.909 | 3.75E−08 | 8.45E−07 |
| C16orf45 | 1.902 | 1.81E−05 | 9.88E−05 |
| MYO7A | 1.898 | 2.72E−07 | 3.75E−06 |
| COL15A1 | 1.897 | 2.08E−08 | 5.64E−07 |
| KIF13A | 1.896 | 4.05E−06 | 3.12E−05 |
| LONRF2 | 1.884 | 0.0004156 | 0.001266 |
| KCNN1 | 1.874 | 4.73E−05 | 0.0002138 |
| TSHZ3 | 1.871 | 5.17E−08 | 1.06E−06 |
| LIPG | 1.866 | 1.12E−07 | 1.85E−06 |
| GJA3 | 1.862 | 1.50E−07 | 2.33E−06 |
| ADAMTS17 | 1.858 | 5.45E−06 | 3.94E−05 |
| GPR68 | 1.85 | 6.90E−06 | 4.72E−05 |
| CTSG | 1.845 | 2.11E−10 | 3.00E−08 |
| NT5E | 1.845 | 2.22E−09 | 1.17E−07 |
| EPHB1 | 1.843 | 1.91E−09 | 1.10E−07 |
| SLC16A10 | 1.841 | 6.17E−06 | 4.35E−05 |
| GBP2 | 1.839 | 7.29E−08 | 1.35E−06 |
| CD14 | 1.838 | 2.30E−08 | 6.09E−07 |
| CXCL1 | 1.828 | 7.54E−05 | 0.000313 |
| FBLN1 | 1.823 | 3.06E−08 | 7.30E−07 |
| EPHA1 | 1.823 | 5.66E−08 | 1.13E−06 |
| MST1 | 1.814 | 7.25E−08 | 1.34E−06 |
| CCL3 | 1.812 | 9.67E−07 | 1.02E−05 |
| EGR2 | 1.807 | 0.0006606 | 0.001866 |
| LOC100130872 | 1.806 | 3.64E−06 | 2.88E−05 |
| SLC29A2 | 1.797 | 3.99E−07 | 5.07E−06 |
| VWF | 1.792 | 0.000105 | 0.0004084 |
| SLCO2B1 | 1.79 | 1.16E−05 | 7.04E−05 |
| PPP1R14C | 1.789 | 6.09E−09 | 2.34E−07 |
| LDLRAD3 | 1.786 | 7.35E−08 | 1.35E−06 |
| TMEM136 | 1.786 | 8.99E−07 | 9.60E−06 |
| IFITM10 | 1.784 | 2.84E−07 | 3.87E−06 |
| CLEC10A | 1.782 | 1.62E−05 | 9.11E−05 |
| POU4F1 | 1.777 | 4.67E−08 | 9.89E−07 |
| RP11-66B24.1 | 1.772 | 9.96E−05 | 0.000391 |
| F2RL3 | 1.759 | 2.97E−06 | 2.45E−05 |
| CLU | 1.758 | 6.18E−07 | 7.09E−06 |
| IL2RG | 1.757 | 1.22E−05 | 7.28E−05 |
| BCL2A1 | 1.753 | 2.64E−06 | 2.22E−05 |
| LTBP1 | 1.752 | 4.22E−06 | 3.23E−05 |
| ABCA6 | 1.751 | 4.37E−05 | 0.0002006 |
| EPPK1 | 1.749 | 4.15E−07 | 5.22E−06 |
| HSD11B1 | 1.748 | 1.41E−10 | 2.46E−08 |
| SH3BGRL2 | 1.745 | 1.52E−07 | 2.35E−06 |
| SIGLEC11 | 1.739 | 2.17E−09 | 1.17E−07 |
| GRM1 | 1.737 | 0.0002518 | 0.0008371 |
| DOCK9 | 1.736 | 6.12E−07 | 7.06E−06 |
| STXBP6 | 1.733 | 4.86E−08 | 1.01E−06 |
| EHD2 | 1.732 | 1.11E−08 | 3.59E−07 |
| ST5 | 1.727 | 1.23E−05 | 7.33E−05 |
| PLEKHA6 | 1.72 | 9.46E−07 | 1.00E−05 |
| SLC26A11 | 1.719 | 3.35E−05 | 0.0001617 |
| SASH1 | 1.717 | 1.48E−10 | 2.50E−08 |
| ATP6V0E2-AS1 | 1.714 | 2.43E−05 | 0.0001255 |
| CDON | 1.711 | 5.73E−08 | 1.14E−06 |
| SLAMF7 | 1.708 | 8.86E−06 | 5.68E−05 |
| DHX32 | 1.705 | 1.23E−07 | 2.00E−06 |
| CEACAM4 | 1.702 | 1.10E−07 | 1.83E−06 |
| SCN8A | 1.7 | 2.66E−05 | 0.0001347 |
| TCF24 | 1.699 | 8.54E−06 | 5.53E−05 |
| SLC35F1 | 1.699 | 1.09E−05 | 6.71E−05 |
| CYP19A1 | 1.699 | 0.0001514 | 0.0005508 |
| SLFN5 | 1.689 | 1.97E−06 | 1.75E−05 |
| SLC1A7 | 1.684 | 1.37E−05 | 8.01E−05 |
| SFRP1 | 1.681 | 2.20E−11 | 1.03E−08 |
| PALLD | 1.678 | 1.68E−05 | 9.37E−05 |
| TREM2 | 1.676 | 5.44E−11 | 1.39E−08 |
| ZFHX2 | 1.675 | 1.95E−05 | 0.0001051 |
| ARAP3 | 1.671 | 2.73E−08 | 6.81E−07 |
| RBP4 | 1.67 | 5.24E−05 | 0.0002325 |
| MAP3K9 | 1.67 | 0.0003543 | 0.001111 |
| NPTX1 | 1.662 | 4.12E−11 | 1.24E−08 |
| MAP7 | 1.662 | 2.11E−07 | 3.05E−06 |
| SORCS2 | 1.655 | 6.21E−07 | 7.12E−06 |
| INHBA-AS1 | 1.653 | 7.62E−05 | 0.0003151 |
| RAB37 | 1.652 | 2.76E−08 | 6.84E−07 |
| CTSD | 1.645 | 1.00E−10 | 2.05E−08 |
| MAPK13 | 1.645 | 7.28E−08 | 1.35E−06 |
| GPRIN3 | 1.644 | 0.0002848 | 0.0009261 |
| ITGB7 | 1.643 | 2.51E−10 | 3.31E−08 |
| C1orf115 | 1.643 | 8.63E−08 | 1.52E−06 |
| SDS | 1.642 | 1.44E−05 | 8.34E−05 |
| TNIK | 1.641 | 1.39E−07 | 2.21E−06 |
| CHI3L1 | 1.64 | 5.99E−11 | 1.51E−08 |
| ALOX5AP | 1.64 | 1.16E−10 | 2.20E−08 |
| PCDHGB4 | 1.64 | 6.26E−08 | 1.21E−06 |
| GJD3 | 1.639 | 8.65E−10 | 6.95E−08 |
| ELANE | 1.635 | 3.09E−10 | 3.68E−08 |
| LOXHD1 | 1.633 | 1.16E−06 | 1.17E−05 |
| CIB2 | 1.63 | 8.68E−05 | 0.00035 |
| KIF5C | 1.629 | 0.0001169 | 0.0004456 |
| GPR84 | 1.627 | 2.60E−06 | 2.20E−05 |
| COL1A1 | 1.626 | 0.001886 | 0.004534 |
| LOC93432 | 1.623 | 0.002678 | 0.006112 |
| P2RX1 | 1.621 | 1.87E−10 | 2.79E−08 |
| SIGLEC9 | 1.621 | 9.12E−05 | 0.0003646 |
| LOC101927686 | 1.618 | 7.84E−05 | 0.0003217 |
| PAQR9 | 1.618 | 0.0001114 | 0.000428 |
| TMEM176A | 1.618 | 0.0002111 | 0.000722 |
| CSF1 | 1.614 | 3.54E−06 | 2.82E−05 |
| SLC37A2 | 1.612 | 3.00E−11 | 1.13E−08 |
| ATP2B3 | 1.612 | 3.74E−07 | 4.82E−06 |
| SPTB | 1.612 | 3.65E−06 | 2.89E−05 |
| LOC654342 | 1.604 | 0.0001543 | 0.0005588 |
| SLC11A1 | 1.6 | 4.72E−10 | 4.85E−08 |
| PTPN14 | 1.6 | 1.62E−08 | 4.67E−07 |
| THSD4 | 1.598 | 1.01E−05 | 6.32E−05 |
| MLNR | 1.591 | 9.73E−07 | 1.03E−05 |
| LRRC3B | 1.589 | 5.36E−08 | 1.09E−06 |
| CPVL | 1.588 | 3.31E−10 | 3.75E−08 |
| SYNPO | 1.587 | 1.20E−07 | 1.97E−06 |
| OAF | 1.585 | 3.89E−11 | 1.22E−08 |
| FOLR2 | 1.58 | 3.29E−07 | 4.36E−06 |
| C3 | 1.575 | 2.19E−10 | 3.00E−08 |
| ID3 | 1.573 | 5.49E−08 | 1.11E−06 |
| STAG3 | 1.572 | 1.75E−07 | 2.64E−06 |
| TOB2P1 | 1.568 | 3.70E−06 | 2.91E−05 |
| VSIG4 | 1.568 | 1.48E−05 | 8.50E−05 |
| FLRT2 | 1.564 | 1.93E−05 | 0.0001041 |
| AMDHD1 | 1.562 | 0.0002881 | 0.0009341 |
| DLGAP1-AS3 | 1.561 | 3.14E−06 | 2.56E−05 |
| RASGRP3 | 1.559 | 1.74E−05 | 9.58E−05 |
| RHOBTB3 | 1.558 | 2.19E−08 | 5.87E−07 |
| GLTID1 | 1.558 | 2.29E−06 | 1.98E−05 |
| EDARADD | 1.557 | 6.43E−06 | 4.50E−05 |
| LOC100289533 | 1.554 | 0.0001803 | 0.0006338 |
| FEZ1 | 1.553 | 1.06E−06 | 1.09E−05 |
| HIST1H2BK | 1.552 | 1.84E−08 | 5.16E−07 |
| ARHGAP31 | 1.551 | 4.41E−07 | 5.45E−06 |
| ANKRD29 | 1.551 | 0.000253 | 0.0008401 |
| ITGA2 | 1.548 | 1.83E−06 | 1.66E−05 |
| DLEU7 | 1.546 | 8.72E−06 | 5.62E−05 |
| CD5 | 1.546 | 0.0002249 | 0.000761 |
| SNAP91 | 1.543 | 9.82E−05 | 0.0003867 |
| CAMK4 | 1.539 | 9.75E−06 | 6.15E−05 |
| TBC1D10C | 1.538 | 4.40E−07 | 5.45E−06 |
| NR2F2 | 1.537 | 4.91E−05 | 0.0002203 |
| C5AR1 | 1.532 | 3.14E−06 | 2.56E−05 |
| BHLHB9 | 1.524 | 8.93E−06 | 5.72E−05 |
| HTR1B | 1.524 | 9.82E−06 | 6.19E−05 |
| ARHGEF3 | 1.523 | 5.86E−09 | 2.28E−07 |
| ZNF358 | 1.523 | 2.40E−07 | 3.40E−06 |
| PAIP2B | 1.511 | 0.000183 | 0.0006409 |
| GREB1 | 1.509 | 1.94E−05 | 0.0001048 |
| FGF2 | 1.509 | 0.001609 | 0.003961 |
| RAB7B | 1.508 | 2.10E−10 | 3.00E−08 |
| CCL3L3 | 1.505 | 1.29E−05 | 7.63E−05 |
| FXYD5 | 1.504 | 4.39E−09 | 1.85E−07 |
| APCDD1 | 1.503 | 5.48E−07 | 6.46E−06 |
| MYO1B | 1.503 | 1.65E−05 | 9.25E−05 |
| LOC101929523 | 1.502 | 3.40E−07 | 4.48E−06 |
| BTBD19 | 1.5 | 0.0006669 | 0.00188 |
| MFGE8 | 1.499 | 2.36E−07 | 3.35E−06 |
| PPARG | 1.498 | 5.42E−10 | 5.16E−08 |
| TMC5 | 1.498 | 1.97E−05 | 0.0001063 |
| ENOX1 | 1.497 | 0.0002732 | 0.000894 |
| GABRR2 | 1.497 | 0.001443 | 0.003605 |
| PCNXL2 | 1.496 | 2.76E−08 | 6.84E−07 |
| RNASE3 | 1.494 | 2.51E−08 | 6.41E−07 |
| CACFD1 | 1.493 | 1.54E−07 | 2.37E−06 |
| GLIDR | 1.493 | 0.0004812 | 0.001432 |
| ATP9A | 1.492 | 2.29E−08 | 6.09E−07 |
| ATP7B | 1.491 | 1.21E−05 | 7.28E−05 |
| GPR37 | 1.491 | 0.0001293 | 0.0004836 |
| PRAM1 | 1.489 | 3.28E−10 | 3.74E−08 |
| ENAH | 1.486 | 1.57E−06 | 1.46E−05 |
| CXCL16 | 1.485 | 7.28E−06 | 4.90E−05 |
| OSMR | 1.485 | 0.0009989 | 0.002639 |
| SLC9A7 | 1.483 | 6.46E−08 | 1.23E−06 |
| MTUS1 | 1.48 | 4.71E−06 | 3.52E−05 |
| LOXL4 | 1.475 | 1.85E−05 | 0.0001006 |
| SLC44A3 | 1.474 | 5.22E−05 | 0.0002322 |
| LOC101928319 | 1.473 | 0.0004236 | 0.001286 |
| PNOC | 1.472 | 1.57E−05 | 8.86E−05 |
| MPO | 1.469 | 8.52E−08 | 1.52E−06 |
| SCN5A | 1.469 | 2.77E−05 | 0.0001388 |
| TMIE | 1.469 | 5.64E−05 | 0.0002461 |
| RABAC1 | 1.466 | 1.36E−08 | 4.14E−07 |
| MLC1 | 1.465 | 1.29E−10 | 2.36E−08 |
| DKK2 | 1.465 | 1.41E−08 | 4.25E−07 |
| REPS2 | 1.465 | 1.81E−05 | 9.90E−05 |
| CHST6 | 1.462 | 1.72E−07 | 2.60E−06 |
| TRPM8 | 1.462 | 4.66E−05 | 0.0002114 |
| MAML3 | 1.461 | 2.04E−06 | 1.80E−05 |
| LOC102724476 | 1.461 | 0.000778 | 0.002137 |
| ZNF215 | 1.457 | 5.28E−06 | 3.85E−05 |
| SAMD5 | 1.457 | 0.0001175 | 0.0004468 |
| TMEM255B | 1.456 | 2.30E−05 | 0.0001202 |
| LRP1 | 1.455 | 2.46E−07 | 3.46E−06 |
| STXBP5 | 1.452 | 3.55E−10 | 3.83E−08 |
| PADI2 | 1.452 | 5.69E−08 | 1.13E−06 |
| DPF3 | 1.451 | 6.32E−07 | 7.21E−06 |
| NKX2-4 | 1.45 | 3.81E−08 | 8.52E−07 |
| SLAMF9 | 1.449 | 1.19E−07 | 1.96E−06 |
| XKR5 | 1.448 | 9.95E−05 | 0.000391 |
| PDGFA | 1.444 | 2.62E−07 | 3.63E−06 |
| HLA-H | 1.443 | 0.0007573 | 0.002089 |
| GOLGA6L9 | 1.442 | 0.004426 | 0.009394 |
| GAS6 | 1.439 | 3.72E−10 | 3.95E−08 |
| IL16 | 1.438 | 1.38E−09 | 9.05E−08 |
| ADAMTS5 | 1.436 | 0.0006533 | 0.001848 |
| STXBP5-AS1 | 1.435 | 1.35E−05 | 7.90E−05 |
| PTK7 | 1.433 | 7.75E−07 | 8.52E−06 |
| FCGRT | 1.43 | 7.90E−10 | 6.66E−08 |
| CEBPE | 1.429 | 3.91E−08 | 8.65E−07 |
| CEACAM21 | 1.426 | 6.77E−08 | 1.28E−06 |
| FLJ36777 | 1.42 | 0.0009528 | 0.002539 |
| TTC9 | 1.418 | 0.0001865 | 0.0006504 |
| CUX2 | 1.417 | 8.43E−07 | 9.09E−06 |
| ADAMTS10 | 1.417 | 4.15E−06 | 3.19E−05 |
| GRN | 1.415 | 2.09E−09 | 1.16E−07 |
| TMC8 | 1.413 | 5.04E−10 | 4.99E−08 |
| WIPI1 | 1.413 | 2.84E−05 | 0.0001415 |
| HAVCR2 | 1.412 | 9.37E−06 | 5.94E−05 |
| LPO | 1.411 | 4.21E−05 | 0.0001947 |
| KIAA0355 | 1.406 | 1.55E−06 | 1.45E−05 |
| SCG3 | 1.406 | 0.0001179 | 0.0004481 |
| LOC101927532 | 1.403 | 0.0001461 | 0.000535 |
| LOC102724571 | 1.401 | 9.86E−05 | 0.0003879 |
| LINC01094 | 1.399 | 9.47E−05 | 0.000376 |
| L1CAM | 1.399 | 0.0009662 | 0.002568 |
| SIAE | 1.398 | 1.11E−09 | 8.19E−08 |
| TESC | 1.396 | 9.84E−08 | 1.68E−06 |
| EMR4P | 1.396 | 2.53E−05 | 0.0001294 |
| TLR1 | 1.395 | 5.02E−07 | 6.03E−06 |
| PAQR5 | 1.395 | 5.51E−07 | 6.49E−06 |
| ZSWIM4 | 1.395 | 7.94E−06 | 5.23E−05 |
| TGM2 | 1.393 | 1.28E−06 | 1.26E−05 |
| PFN2 | 1.391 | 4.09E−05 | 0.0001906 |
| LINC00883 | 1.391 | 0.001198 | 0.003072 |
| VIT | 1.389 | 1.36E−09 | 9.05E−08 |
| APBB1 | 1.389 | 3.84E−07 | 4.92E−06 |
| SERPINF2 | 1.387 | 0.0002316 | 0.0007798 |
| MYO3B | 1.385 | 0.0005477 | 0.001596 |
| DLL3 | 1.385 | 0.001184 | 0.003041 |
| PCDHGB7 | 1.383 | 0.0003918 | 0.001207 |
| TDO2 | 1.382 | 9.80E−05 | 0.0003864 |
| KCNK2 | 1.38 | 0.001094 | 0.002843 |
| MST1L | 1.379 | 0.001537 | 0.003805 |
| LOX | 1.376 | 0.0008275 | 0.002253 |
| ITGA3 | 1.375 | 4.93E−07 | 5.94E−06 |
| ECSCR | 1.375 | 0.0008299 | 0.002258 |
| DEFB1 | 1.374 | 2.56E−08 | 6.53E−07 |
| FAM198B | 1.374 | 7.61E−05 | 0.0003149 |
| HEXA | 1.372 | 1.33E−10 | 2.38E−08 |
| CSF1R | 1.372 | 7.14E−10 | 6.22E−08 |
| TNN | 1.371 | 4.26E−09 | 1.82E−07 |
| C1S | 1.371 | 2.74E−07 | 3.76E−06 |
| PTCH2 | 1.37 | 0.0001081 | 0.000418 |
| CORO2A | 1.368 | 8.94E−10 | 7.02E−08 |
| ACAP1 | 1.368 | 1.61E−06 | 1.50E−05 |
| NRN1 | 1.365 | 8.70E−09 | 3.00E−07 |
| KLHL30 | 1.362 | 3.32E−08 | 7.77E−07 |
| CRYBB1 | 1.36 | 4.45E−07 | 5.48E−06 |
| RP11-426L16.8 | 1.36 | 0.0001007 | 0.0003945 |
| EPHA1-AS1 | 1.359 | 3.60E−05 | 0.0001722 |
| TLR5 | 1.358 | 1.28E−06 | 1.26E−05 |
| C11orf63 | 1.352 | 1.46E−07 | 2.28E−06 |
| ITGB2 | 1.349 | 3.50E−10 | 3.83E−08 |
| CRISPLD2 | 1.349 | 0.001008 | 0.002661 |
| FHL3 | 1.347 | 1.19E−06 | 1.19E−05 |
| SYNGR1 | 1.341 | 1.21E−08 | 3.81E−07 |
| AC147651.4 | 1.339 | 5.72E−08 | 1.14E−06 |
| PCDHGA11 | 1.337 | 5.90E−06 | 4.20E−05 |
| MAP3K15 | 1.337 | 5.86E−05 | 0.0002543 |
| TMOD2 | 1.335 | 0.0007551 | 0.002084 |
| HCST | 1.333 | 5.83E−08 | 1.15E−06 |
| ADAMTS1 | 1.33 | 2.55E−09 | 1.27E−07 |
| PDK4 | 1.33 | 7.32E−06 | 4.92E−05 |
| LOC101927085 | 1.33 | 4.59E−05 | 0.000209 |
| SCRN1 | 1.329 | 6.97E−08 | 1.31E−06 |
| CHST2 | 1.329 | 1.26E−06 | 1.24E−05 |
| ARHGAP20 | 1.322 | 1.03E−05 | 6.41E−05 |
| MEX3B | 1.321 | 0.001737 | 0.004224 |
| CD300LG | 1.321 | 0.001838 | 0.00444 |
| GAA | 1.319 | 2.83E−10 | 3.52E−08 |
| CD52 | 1.318 | 2.07E−08 | 5.63E−07 |
| ZMYND12 | 1.318 | 3.04E−06 | 2.50E−05 |
| PDE6G | 1.316 | 0.0001232 | 0.0004655 |
| MRVI1 | 1.316 | 0.0001275 | 0.0004783 |
| SMPD5 | 1.316 | 0.0005198 | 0.001527 |
| ALOX5 | 1.315 | 3.52E−09 | 1.57E−07 |
| TNXB | 1.313 | 1.54E−05 | 8.75E−05 |
| ANKDD1A | 1.313 | 0.0002039 | 0.0007014 |
| PMEL | 1.312 | 0.0009432 | 0.002518 |
| VASH1 | 1.31 | 1.73E−10 | 2.64E−08 |
| PLCB1 | 1.304 | 0.0006114 | 0.001748 |
| TGFBI | 1.3 | 2.75E−10 | 3.46E−08 |
| PLD4 | 1.299 | 7.57E−10 | 6.51E−08 |
| ADORA3 | 1.299 | 8.47E−07 | 9.12E−06 |
| HTRA3 | 1.297 | 5.06E−05 | 0.000226 |
| CAMKV | 1.296 | 4.95E−06 | 3.66E−05 |
| SPTBN1 | 1.295 | 4.49E−08 | 9.62E−07 |
| NUAK1 | 1.295 | 0.0001362 | 0.0005048 |
| PDIA5 | 1.294 | 5.61E−10 | 5.22E−08 |
| KLHL29 | 1.292 | 1.31E−07 | 2.11E−06 |
| NDRG2 | 1.291 | 9.52E−09 | 3.22E−07 |
| TPP1 | 1.287 | 1.07E−09 | 7.98E−08 |
| DERL3 | 1.287 | 6.26E−07 | 7.15E−06 |
| LOC101929151 | 1.287 | 0.001847 | 0.004457 |
| IGSF1 | 1.285 | 2.67E−08 | 6.71E−07 |
| ARRDC4 | 1.284 | 8.52E−10 | 6.95E−08 |
| CAV1 | 1.284 | 9.08E−08 | 1.59E−06 |
| CD247 | 1.281 | 0.000882 | 0.002376 |
| NAV1 | 1.28 | 3.82E−07 | 4.90E−06 |
| PIP5K1B | 1.279 | 1.24E−07 | 2.01E−06 |
| GPR133 | 1.279 | 3.13E−06 | 2.56E−05 |
| NFE2 | 1.277 | 7.49E−09 | 2.71E−07 |
| LOC101929667 | 1.277 | 1.63E−05 | 9.13E−05 |
| C2 | 1.274 | 1.57E−08 | 4.58E−07 |
| PSMG3-AS1 | 1.274 | 3.97E−08 | 8.75E−07 |
| CMAHP | 1.274 | 0.000189 | 0.0006577 |
| C1orf95 | 1.273 | 0.0002889 | 0.0009359 |
| FITM2 | 1.272 | 1.41E−05 | 8.19E−05 |
| CRLF2 | 1.272 | 2.83E−05 | 0.0001414 |
| LOC101928101 | 1.272 | 5.50E−05 | 0.0002417 |
| RAPGEF4 | 1.271 | 0.0009337 | 0.002496 |
| ARHGAP24 | 1.269 | 1.27E−05 | 7.55E−05 |
| S100A8 | 1.269 | 1.00E−05 | 6.30E−05 |
| C1orf200 | 1.268 | 4.83E−07 | 5.85E−06 |
| TMEM86A | 1.267 | 2.06E−06 | 1.81E−05 |
| IFI30 | 1.266 | 2.74E−09 | 1.35E−07 |
| ARAP2 | 1.266 | 5.65E−06 | 4.06E−05 |
| RAB38 | 1.265 | 0.0004025 | 0.001233 |
| RAB17 | 1.262 | 0.0001079 | 0.0004172 |
| SLC16A2 | 1.261 | 2.66E−05 | 0.0001347 |
| SGSH | 1.26 | 2.49E−05 | 0.0001281 |
| SPATC1 | 1.26 | 0.00127 | 0.00323 |
| SMPD1 | 1.258 | 2.12E−09 | 1.17E−07 |
| OLIG1 | 1.256 | 4.66E−07 | 5.67E−06 |
| ZNF536 | 1.256 | 9.48E−05 | 0.0003763 |
| FAM26F | 1.256 | 0.0002577 | 0.0008532 |
| ITGAX | 1.254 | 8.38E−09 | 2.93E−07 |
| IL1RN | 1.251 | 2.11E−08 | 5.70E−07 |
| CETP | 1.249 | 0.000423 | 0.001285 |
| TCHH | 1.248 | 6.09E−08 | 1.19E−06 |
| FBXO25 | 1.246 | 5.20E−08 | 1.07E−06 |
| CASS4 | 1.245 | 2.02E−06 | 1.78E−05 |
| TSPAN6 | 1.243 | 0.0005114 | 0.001506 |
| NWD1 | 1.241 | 0.007163 | 0.01431 |
| CDK20 | 1.237 | 1.04E−08 | 3.42E−07 |
| KLF15 | 1.237 | 1.48E−06 | 1.40E−05 |
| FHDC1 | 1.237 | 1.20E−05 | 7.24E−05 |
| NOMO3 | 1.232 | 2.21E−07 | 3.15E−06 |
| KIAA1324L | 1.232 | 1.85E−06 | 1.68E−05 |
| FOXO3B | 1.231 | 0.0001646 | 0.0005885 |
| ELFN2 | 1.23 | 2.75E−05 | 0.000138 |
| QPCT | 1.229 | 5.66E−07 | 6.61E−06 |
| MRC2 | 1.228 | 8.79E−10 | 6.98E−08 |
| FAM150A | 1.228 | 7.20E−05 | 0.0003002 |
| IGFBPL1 | 1.227 | 3.95E−09 | 1.71E−07 |
| LOC101929014 | 1.227 | 0.0003783 | 0.001172 |
| DZIP1 | 1.226 | 3.18E−08 | 7.55E−07 |
| TPBG | 1.224 | 2.14E−09 | 1.17E−07 |
| ALPK3 | 1.223 | 8.22E−08 | 1.48E−06 |
| FAM229B | 1.222 | 1.57E−05 | 8.89E−05 |
| FBXO32 | 1.22 | 0.0003136 | 0.001005 |
| VWA7 | 1.22 | 0.001659 | 0.00406 |
| CILP2 | 1.219 | 0.0001243 | 0.0004686 |
| HHLA3 | 1.218 | 1.78E−05 | 9.76E−05 |
| FCAMR | 1.217 | 1.21E−05 | 7.27E−05 |
| TBXAS1 | 1.216 | 1.91E−06 | 1.72E−05 |
| MGAT4A | 1.215 | 2.79E−09 | 1.36E−07 |
| SPN | 1.215 | 1.26E−08 | 3.92E−07 |
| SLC22A4 | 1.214 | 1.18E−08 | 3.74E−07 |
| COLGALT2 | 1.213 | 8.47E−08 | 1.51E−06 |
| RASAL2-AS1 | 1.213 | 0.0006738 | 0.001897 |
| LOC100507316 | 1.212 | 3.02E−05 | 0.0001489 |
| P4HA3 | 1.211 | 0.0006099 | 0.001745 |
| HLA-L | 1.208 | 1.27E−06 | 1.25E−05 |
| PRRT4 | 1.202 | 7.78E−07 | 8.52E−06 |
| CCDC64 | 1.201 | 8.92E−10 | 7.02E−08 |
| LBH | 1.2 | 1.73E−06 | 1.58E−05 |
| DAAM1 | 1.2 | 2.54E−06 | 2.15E−05 |
| ARL4C | 1.2 | 1.52E−05 | 8.67E−05 |
| C4orf19 | 1.2 | 7.79E−05 | 0.0003204 |
| PLEKHA8 | 1.197 | 6.22E−08 | 1.20E−06 |
| TSGA10 | 1.197 | 9.69E−05 | 0.000383 |
| NFAM1 | 1.195 | 6.02E−09 | 2.32E−07 |
| TSPEAR-AS1 | 1.194 | 4.36E−05 | 0.0002004 |
| MKLN1-AS | 1.192 | 0.000241 | 0.0008054 |
| SOGA2 | 1.186 | 1.25E−07 | 2.03E−06 |
| SYN2 | 1.186 | 2.65E−05 | 0.0001345 |
| CFP | 1.185 | 3.99E−08 | 8.77E−07 |
| CCDC103 | 1.185 | 1.05E−06 | 1.09E−05 |
| LINC00886 | 1.185 | 6.39E−05 | 0.0002726 |
| FAM174B | 1.184 | 1.16E−06 | 1.17E−05 |
| FUZ | 1.184 | 0.002052 | 0.004871 |
| FMN1 | 1.183 | 2.39E−05 | 0.0001238 |
| NOMO1 | 1.182 | 1.98E−09 | 1.12E−07 |
| EFNA5 | 1.178 | 0.0004033 | 0.001235 |
| CUL7 | 1.177 | 4.63E−09 | 1.94E−07 |
| ZNF219 | 1.177 | 2.99E−06 | 2.47E−05 |
| TMEM71 | 1.176 | 2.01E−05 | 0.0001082 |
| LURAP1L | 1.176 | 0.0003713 | 0.001153 |
| CAV2 | 1.171 | 2.49E−05 | 0.0001278 |
| DNAH6 | 1.171 | 0.0001715 | 0.0006084 |
| PLEKHA7 | 1.171 | 0.0003709 | 0.001153 |
| HLX-AS1 | 1.171 | 0.0006684 | 0.001883 |
| SCPEP1 | 1.17 | 1.91E−09 | 1.10E−07 |
| ERG | 1.168 | 2.82E−05 | 0.0001411 |
| ABCB11 | 1.168 | 0.001081 | 0.002817 |
| RASAL3 | 1.167 | 4.46E−08 | 9.59E−07 |
| PNCK | 1.167 | 8.04E−05 | 0.0003284 |
| CSPG4 | 1.166 | 6.17E−07 | 7.08E−06 |
| CLIP4 | 1.166 | 0.0001964 | 0.0006804 |
| HLA-G | 1.165 | 0.0004363 | 0.001318 |
| TGM5 | 1.162 | 5.56E−09 | 2.21E−07 |
| SYTL1 | 1.162 | 1.20E−07 | 1.98E−06 |
| KIF9-AS1 | 1.161 | 0.001227 | 0.003135 |
| CNKSR3 | 1.16 | 8.29E−07 | 8.98E−06 |
| TMEM150A | 1.159 | 2.54E−05 | 0.0001299 |
| SLC51A | 1.159 | 0.0001005 | 0.000394 |
| ARSA | 1.158 | 2.99E−09 | 1.40E−07 |
| C9orf9 | 1.157 | 0.0004112 | 0.001256 |
| EGR4 | 1.157 | 0.001401 | 0.003513 |
| CACNB1 | 1.155 | 3.84E−07 | 4.92E−06 |
| NOMO2 | 1.149 | 6.59E−08 | 1.25E−06 |
| RNF32 | 1.148 | 2.17E−05 | 0.0001152 |
| NPM2 | 1.147 | 3.29E−06 | 2.66E−05 |
| EDEM2 | 1.146 | 6.20E−09 | 2.36E−07 |
| LOC101927594 | 1.144 | 0.001773 | 0.004297 |
| LOC100129917 | 1.142 | 0.0001481 | 0.0005409 |
| GSPT2 | 1.141 | 7.36E−07 | 8.17E−06 |
| LINC00654 | 1.14 | 0.0001046 | 0.0004066 |
| ATP6V0A1 | 1.139 | 3.13E−08 | 7.46E−07 |
| MAGED1 | 1.139 | 2.18E−07 | 3.13E−06 |
| COL6A1 | 1.138 | 3.06E−09 | 1.42E−07 |
| PPP1R13B | 1.137 | 2.09E−05 | 0.0001114 |
| RNF135 | 1.136 | 8.80E−09 | 3.03E−07 |
| HIST2H2BE | 1.136 | 7.25E−05 | 0.0003022 |
| SBK1 | 1.135 | 0.0111 | 0.02092 |
| CD180 | 1.134 | 2.84E−07 | 3.87E−06 |
| MSC | 1.133 | 6.96E−07 | 7.79E−06 |
| SAMD15 | 1.133 | 6.95E−05 | 0.000292 |
| CNIH3 | 1.131 | 3.70E−05 | 0.0001761 |
| WASF1 | 1.13 | 9.16E−09 | 3.13E−07 |
| DST | 1.13 | 2.25E−06 | 1.96E−05 |
| TCTN1 | 1.129 | 5.47E−07 | 6.45E−06 |
| PIGT | 1.128 | 4.60E−08 | 9.81E−07 |
| PLOD3 | 1.127 | 3.77E−09 | 1.65E−07 |
| WDR35 | 1.126 | 1.42E−06 | 1.36E−05 |
| KCP | 1.126 | 1.18E−05 | 7.12E−05 |
| AGPAT9 | 1.126 | 2.49E−05 | 0.0001281 |
| KCNK13 | 1.126 | 0.0002343 | 0.0007869 |
| TTC28 | 1.125 | 3.87E−06 | 3.01E−05 |
| CNRIP1 | 1.124 | 4.03E−06 | 3.12E−05 |
| PAN3-AS1 | 1.123 | 0.0001011 | 0.0003955 |
| PKIG | 1.123 | 0.0001401 | 0.0005167 |
| SIRPB1 | 1.122 | 1.53E−07 | 2.37E−06 |
| IRAK2 | 1.122 | 6.90E−06 | 4.72E−05 |
| CGNL1 | 1.122 | 1.35E−05 | 7.91E−05 |
| GPR161 | 1.122 | 0.0003287 | 0.001044 |
| HLA-DMA | 1.119 | 3.12E−06 | 2.55E−05 |
| DKKL1 | 1.119 | 0.002296 | 0.005346 |
| HSPB7 | 1.118 | 6.01E−09 | 2.32E−07 |
| RAP1GAP2 | 1.117 | 8.58E−08 | 1.52E−06 |
| NHLRC1 | 1.116 | 7.56E−05 | 0.0003135 |
| MYO1E | 1.115 | 5.30E−08 | 1.08E−06 |
| FKBP9 | 1.114 | 9.57E−09 | 3.22E−07 |
| PC | 1.114 | 1.71E−06 | 1.57E−05 |
| PTPN21 | 1.114 | 0.000369 | 0.001147 |
| SYDE2 | 1.111 | 2.04E−06 | 1.80E−05 |
| GRIN3A | 1.111 | 2.55E−05 | 0.0001302 |
| ELFN1 | 1.111 | 0.003294 | 0.007294 |
| FBP1 | 1.11 | 4.84E−09 | 2.00E−07 |
| PDGFD | 1.11 | 8.13E−09 | 2.86E−07 |
| MORC2-AS1 | 1.11 | 3.28E−05 | 0.0001587 |
| NRXN2 | 1.109 | 0.001538 | 0.003805 |
| TIMP1 | 1.105 | 4.85E−08 | 1.01E−06 |
| ULBP3 | 1.105 | 4.25E−05 | 0.0001959 |
| EPHB3 | 1.105 | 6.52E−05 | 0.000277 |
| ANXA8L1 | 1.105 | 0.0004254 | 0.00129 |
| PNPLA1 | 1.104 | 1.13E−05 | 6.89E−05 |
| RIMS2 | 1.099 | 0.001208 | 0.003092 |
| SIX4 | 1.096 | 0.003141 | 0.007012 |
| ZCCHC5 | 1.095 | 0.002459 | 0.005676 |
| EBI3 | 1.095 | 0.005767 | 0.01184 |
| CTSA | 1.094 | 1.04E−08 | 3.43E−07 |
| GGT7 | 1.094 | 4.30E−07 | 5.37E−06 |
| NEB | 1.094 | 1.20E−05 | 7.24E−05 |
| VIPR1 | 1.094 | 0.0001939 | 0.0006725 |
| NDST1 | 1.092 | 4.34E−08 | 9.39E−07 |
| OSBP2 | 1.091 | 0.0001994 | 0.0006891 |
| LOC101927200 | 1.091 | 0.001452 | 0.003625 |
| IL21R | 1.09 | 1.40E−07 | 2.22E−06 |
| C6orf132 | 1.09 | 0.0007519 | 0.002077 |
| RNASET2 | 1.088 | 4.79E−09 | 1.99E−07 |
| PTPRU | 1.085 | 6.11E−07 | 7.05E−06 |
| TNFSF14 | 1.083 | 7.73E−08 | 1.41E−06 |
| TDRKH | 1.083 | 2.86E−06 | 2.38E−05 |
| POMC | 1.082 | 3.49E−07 | 4.58E−06 |
| LOC101929131 | 1.08 | 0.01364 | 0.02498 |
| DUSP4 | 1.079 | 1.57E−06 | 1.47E−05 |
| FBLL1 | 1.078 | 0.0002888 | 0.0009356 |
| HLA-DQB1 | 1.077 | 2.45E−08 | 6.37E−07 |
| CNTNAP3B | 1.077 | 4.32E−07 | 5.38E−06 |
| LOC100505771 | 1.077 | 1.13E−05 | 6.90E−05 |
| HVCN1 | 1.076 | 7.57E−08 | 1.38E−06 |
| TFAP2A | 1.076 | 0.0006245 | 0.001781 |
| FXYD6 | 1.075 | 0.0002344 | 0.0007873 |
| TBL1X | 1.073 | 8.50E−08 | 1.52E−06 |
| HLA-DRB1 | 1.07 | 2.57E−09 | 1.27E−07 |
| PLBD2 | 1.07 | 6.75E−07 | 7.61E−06 |
| HLA-DRB6 | 1.07 | 0.0002624 | 0.0008656 |
| PPP1R13L | 1.069 | 8.15E−05 | 0.0003323 |
| FRMD3 | 1.068 | 7.34E−08 | 1.35E−06 |
| ATP1A3 | 1.068 | 4.99E−06 | 3.68E−05 |
| PFKM | 1.068 | 2.82E−05 | 0.000141 |
| FAT4 | 1.067 | 0.008338 | 0.0163 |
| CALCRL | 1.066 | 0.001272 | 0.003233 |
| TMEM150C | 1.065 | 8.59E−05 | 0.0003468 |
| GRIK2 | 1.062 | 0.002893 | 0.006533 |
| LOC100129461 | 1.061 | 0.005584 | 0.01152 |
| HHAT | 1.06 | 3.77E−06 | 2.96E−05 |
| ANXA8 | 1.058 | 4.68E−06 | 3.50E−05 |
| C21orf90 | 1.055 | 0.001237 | 0.003158 |
| LOC101928291 | 1.055 | 0.001397 | 0.003505 |
| LOC283887 | 1.055 | 0.004301 | 0.009174 |
| QPRT | 1.054 | 2.94E−09 | 1.40E−07 |
| ATP6V1C2 | 1.054 | 1.70E−05 | 9.43E−05 |
| AK9 | 1.054 | 4.59E−05 | 0.0002092 |
| TXLNB | 1.054 | 0.004728 | 0.009938 |
| PLEC | 1.053 | 5.81E−07 | 6.75E−06 |
| CPE | 1.052 | 1.60E−07 | 2.45E−06 |
| IL1B | 1.051 | 0.0004219 | 0.001282 |
| LRRC39 | 1.051 | 0.01146 | 0.0215 |
| HEXB | 1.05 | 2.37E−09 | 1.22E−07 |
| ACRBP | 1.049 | 2.92E−06 | 2.42E−05 |
| SYNE1 | 1.049 | 3.60E−06 | 2.86E−05 |
| HLX | 1.048 | 8.09E−08 | 1.46E−06 |
| RAB3IL1 | 1.047 | 5.27E−08 | 1.08E−06 |
| KIAA1024 | 1.047 | 6.10E−05 | 0.0002631 |
| EXOC6B | 1.046 | 8.65E−07 | 9.30E−06 |
| LRRC6 | 1.046 | 0.0004897 | 0.001454 |
| PCDHGA8 | 1.046 | 0.0006794 | 0.00191 |
| MX2 | 1.042 | 1.12E−08 | 3.59E−07 |
| HIST1H1C | 1.042 | 1.60E−05 | 9.02E−05 |
| C20orf197 | 1.042 | 3.21E−05 | 0.0001564 |
| IL1R1 | 1.04 | 0.000112 | 0.00043 |
| CLUHP3 | 1.039 | 0.002371 | 0.005496 |
| NEU1 | 1.038 | 4.92E−09 | 2.02E−07 |
| PDE7B | 1.038 | 7.85E−08 | 1.43E−06 |
| PGR | 1.038 | 9.19E−08 | 1.60E−06 |
| GLDC | 1.038 | 0.0002754 | 0.0008998 |
| PHC1 | 1.036 | 1.57E−05 | 8.86E−05 |
| TBC1D9 | 1.035 | 2.56E−07 | 3.56E−06 |
| NUDT16P1 | 1.033 | 2.21E−05 | 0.0001165 |
| DNAJC6 | 1.033 | 0.0003498 | 0.0011 |
| LOC101928143 | 1.033 | 0.0008433 | 0.002287 |
| FBXO27 | 1.032 | 6.26E−05 | 0.0002683 |
| SLC1A4 | 1.031 | 2.96E−08 | 7.15E−07 |
| OLFML3 | 1.03 | 1.23E−07 | 2.00E−06 |
| SHH | 1.03 | 0.01058 | 0.02005 |
| KCND1 | 1.028 | 0.0003542 | 0.001111 |
| FAM159A | 1.028 | 0.001986 | 0.004736 |
| RGS22 | 1.027 | 0.002406 | 0.005569 |
| TMEM9B-AS1 | 1.026 | 0.0007017 | 0.00196 |
| UGDH-AS1 | 1.026 | 0.001501 | 0.003727 |
| RUNDC3A | 1.026 | 0.001846 | 0.004455 |
| GANAB | 1.025 | 3.73E−09 | 1.65E−07 |
| TBX3 | 1.025 | 2.34E−06 | 2.01E−05 |
| TMEM45A | 1.022 | 1.42E−07 | 2.24E−06 |
| ASIC1 | 1.021 | 1.20E−07 | 1.97E−06 |
| PRKCSH | 1.019 | 1.41E−08 | 4.25E−07 |
| BEX4 | 1.019 | 8.59E−05 | 0.0003468 |
| ABCC5 | 1.018 | 2.72E−06 | 2.28E−05 |
| TNFRSF10C | 1.018 | 0.001453 | 0.003627 |
| HLA-DMB | 1.017 | 0.0005502 | 0.001602 |
| DPYD | 1.016 | 2.46E−09 | 1.24E−07 |
| QSOX1 | 1.015 | 1.35E−08 | 4.13E−07 |
| MIR4477B | 1.014 | 0.003358 | 0.00741 |
| LOC101929004 | 1.013 | 0.001202 | 0.00308 |
| TUBA1A | 1.013 | 0.007814 | 0.01542 |
| RAB11FIP4 | 1.012 | 1.61E−06 | 1.49E−05 |
| NEK8 | 1.011 | 1.23E−06 | 1.22E−05 |
| HYDIN2 | 1.01 | 0.001678 | 0.0041 |
| HTATSF1P2 | 1.009 | 6.15E−06 | 4.35E−05 |
| PMFBP1 | 1.009 | 0.0004869 | 0.001446 |
| SRCIN1 | 1.009 | 0.000787 | 0.002158 |
| CSF3R | 1.008 | 1.24E−09 | 8.69E−08 |
| ENG | 1.008 | 1.31E−07 | 2.11E−06 |
| RAB3C | 1.008 | 0.0002441 | 0.0008149 |
| TRPM2 | 1.006 | 1.15E−09 | 8.27E−08 |
| NCALD | 1.006 | 5.53E−08 | 1.11E−06 |
| SMO | 1.006 | 0.0001045 | 0.0004066 |
| GABRB2 | 1.006 | 0.0001497 | 0.0005459 |
| SCAMP5 | 1.004 | 2.25E−08 | 6.01E−07 |
| LOC100507507 | 1.004 | 6.23E−07 | 7.13E−06 |
| ST6GALNAC2 | 1.004 | 1.54E−05 | 8.78E−05 |
| LOC101927578 | 1.003 | 0.003271 | 0.007257 |
| FMNL2 | 1.002 | 1.96E−06 | 1.74E−05 |
| ARMC2 | 1.002 | 2.60E−05 | 0.0001323 |
| RNASE6 | 1.001 | 0.0002159 | 0.0007353 |
| PSMC6 | −1.001 | 5.23E−05 | 0.0002325 |
| SAR1A | −1.003 | 5.90E−09 | 2.29E−07 |
| CERS1 | −1.003 | 1.45E−05 | 8.35E−05 |
| ARID2 | −1.004 | 6.86E−08 | 1.29E−06 |
| LGI2 | −1.004 | 0.0004711 | 0.001406 |
| C5orf51 | −1.005 | 7.67E−09 | 2.74E−07 |
| ZFP14 | −1.006 | 7.43E−06 | 4.98E−05 |
| NLGN2 | −1.007 | 0.0008716 | 0.002352 |
| RP11-344B5.2 | −1.007 | 0.001565 | 0.003863 |
| LY6G5C | −1.007 | 0.002878 | 0.006506 |
| PPP2R2D | −1.008 | 1.33E−08 | 4.09E−07 |
| PPAP2A | −1.008 | 0.0009595 | 0.002552 |
| CCT5 | −1.009 | 3.47E−09 | 1.57E−07 |
| IKZF5 | −1.009 | 4.29E−08 | 9.33E−07 |
| RBM39 | −1.009 | 5.20E−07 | 6.21E−06 |
| USP18 | −1.009 | 3.32E−06 | 2.67E−05 |
| EDRF1 | −1.01 | 3.77E−08 | 8.47E−07 |
| CRYZ | −1.012 | 1.46E−06 | 1.40E−05 |
| PYROXD2 | −1.012 | 2.28E−05 | 0.0001198 |
| NFKB2 | −1.014 | 4.81E−08 | 1.01E−06 |
| FAM111B | −1.014 | 6.15E−08 | 1.19E−06 |
| UBTD1 | −1.014 | 9.90E−08 | 1.69E−06 |
| ZSCAN12 | −1.014 | 3.60E−05 | 0.0001721 |
| KRCC1 | −1.015 | 2.77E−05 | 0.0001389 |
| NHLRC2 | −1.017 | 1.49E−06 | 1.41E−05 |
| OSBPL8 | −1.018 | 4.36E−07 | 5.43E−06 |
| PAPD7 | −1.019 | 2.88E−08 | 6.99E−07 |
| RBM17 | −1.02 | 2.48E−09 | 1.24E−07 |
| LOC101928092 | −1.021 | 0.0001068 | 0.0004139 |
| VDAC2 | −1.023 | 3.79E−08 | 8.50E−07 |
| ZNF492 | −1.023 | 2.46E−06 | 2.10E−05 |
| ZNF682 | −1.023 | 1.11E−05 | 6.77E−05 |
| NCKAP1 | −1.023 | 0.0003293 | 0.001045 |
| CALM2 | −1.025 | 1.73E−05 | 9.57E−05 |
| PGAM4 | −1.025 | 4.19E−05 | 0.0001943 |
| DUSP5P1 | −1.026 | 8.59E−05 | 0.0003468 |
| LIPJ | −1.026 | 0.0001661 | 0.0005925 |
| SCD5 | −1.027 | 1.91E−06 | 1.72E−05 |
| PDCD4 | −1.028 | 1.09E−08 | 3.56E−07 |
| NAP1L1 | −1.029 | 6.16E−09 | 2.36E−07 |
| COQ3 | −1.03 | 1.13E−06 | 1.14E−05 |
| EIF3E | −1.03 | 7.06E−05 | 0.0002955 |
| SRRM3 | −1.031 | 7.92E−05 | 0.0003244 |
| GALR2 | −1.031 | 0.001288 | 0.003266 |
| MYNN | −1.032 | 1.35E−05 | 7.93E−05 |
| LOC102724002 | −1.032 | 8.06E−05 | 0.0003292 |
| LGR4 | −1.033 | 3.76E−09 | 1.65E−07 |
| ZNF845 | −1.033 | 1.56E−06 | 1.46E−05 |
| MLLT10 | −1.034 | 3.09E−09 | 1.43E−07 |
| FAM21C | −1.037 | 5.17E−09 | 2.08E−07 |
| ZNF254 | −1.037 | 5.42E−06 | 3.93E−05 |
| TIAL1 | −1.038 | 1.28E−09 | 8.89E−08 |
| AVPI1 | −1.038 | 0.0001709 | 0.0006068 |
| NUP37 | −1.04 | 3.75E−07 | 4.83E−06 |
| SGOL2 | −1.04 | 3.15E−06 | 2.56E−05 |
| TRMT10C | −1.04 | 7.20E−06 | 4.86E−05 |
| VIM-AS1 | −1.04 | 1.56E−05 | 8.85E−05 |
| DPM1 | −1.04 | 3.52E−05 | 0.000169 |
| ADO | −1.041 | 8.71E−09 | 3.00E−07 |
| VPS29 | −1.041 | 1.52E−06 | 1.43E−05 |
| NDUFAF7 | −1.041 | 1.15E−05 | 6.99E−05 |
| FBXO4 | −1.042 | 1.47E−06 | 1.40E−05 |
| CYSLTR1 | −1.043 | 1.86E−06 | 1.68E−05 |
| DDX11-AS1 | −1.043 | 0.0006734 | 0.001896 |
| AGAP6 | −1.044 | 1.64E−05 | 9.21E−05 |
| SDHAP3 | −1.045 | 2.81E−06 | 2.34E−05 |
| NDUFS4 | −1.046 | 2.48E−05 | 0.0001275 |
| ZNF239 | −1.047 | 6.91E−07 | 7.75E−06 |
| MATR3 | −1.047 | 5.41E−07 | 6.40E−06 |
| MRPS28 | −1.048 | 2.54E−07 | 3.54E−06 |
| NET1 | −1.049 | 7.01E−09 | 2.57E−07 |
| FAM175A | −1.049 | 3.93E−06 | 3.05E−05 |
| JKAMP | −1.05 | 2.01E−06 | 1.78E−05 |
| KIF18A | −1.05 | 2.35E−05 | 0.0001222 |
| RPL41 | −1.051 | 7.20E−08 | 1.34E−06 |
| CKS2 | −1.051 | 1.55E−06 | 1.46E−05 |
| FCGR2C | −1.051 | 0.0003092 | 0.0009929 |
| CETN3 | −1.052 | 6.52E−06 | 4.53E−05 |
| CASP3 | −1.052 | 1.05E−05 | 6.53E−05 |
| SRGN | −1.053 | 2.28E−06 | 1.98E−05 |
| SFXN2 | −1.054 | 1.75E−08 | 4.97E−07 |
| SNORA81 | −1.054 | 0.0008529 | 0.002309 |
| TMC4 | −1.054 | 0.001881 | 0.004526 |
| ZNF37BP | −1.057 | 0.0001557 | 0.0005628 |
| PDCD6 | −1.059 | 5.63E−09 | 2.21E−07 |
| TMEM180 | −1.059 | 2.75E−06 | 2.30E−05 |
| SEPHS1 | −1.063 | 3.28E−09 | 1.50E−07 |
| GIMAP2 | −1.063 | 2.54E−06 | 2.15E−05 |
| DNAAF3 | −1.063 | 0.000751 | 0.002075 |
| METRN | −1.066 | 1.64E−06 | 1.52E−05 |
| TCF7 | −1.067 | 1.12E−06 | 1.14E−05 |
| HIF1AN | −1.068 | 7.55E−09 | 2.72E−07 |
| GLUD1 | −1.069 | 1.41E−09 | 9.16E−08 |
| ZCRB1 | −1.069 | 2.12E−08 | 5.72E−07 |
| SEC31B | −1.069 | 0.001401 | 0.003513 |
| BDH2 | −1.07 | 2.75E−07 | 3.76E−06 |
| BMI1 | −1.072 | 2.38E−06 | 2.04E−05 |
| LKAAEAR1 | −1.074 | 0.006881 | 0.01383 |
| NUTM2B | −1.074 | 0.008797 | 0.01704 |
| IPO8 | −1.076 | 4.34E−09 | 1.85E−07 |
| LRRC20 | −1.076 | 1.27E−07 | 2.06E−06 |
| HPS6 | −1.077 | 2.43E−08 | 6.35E−07 |
| LOC101928000 | −1.078 | 2.77E−05 | 0.0001388 |
| FBXL15 | −1.079 | 1.75E−07 | 2.64E−06 |
| RUFY4 | −1.079 | 0.0003461 | 0.001092 |
| HOXB13 | −1.083 | 0.003352 | 0.007397 |
| TVP23B | −1.084 | 7.69E−05 | 0.0003174 |
| LOC101929021 | −1.086 | 1.12E−06 | 1.14E−05 |
| BEND4 | −1.086 | 4.28E−06 | 3.27E−05 |
| MAP3K8 | −1.087 | 2.74E−05 | 0.0001376 |
| XPNPEP1 | −1.088 | 1.87E−09 | 1.09E−07 |
| ZNF100 | −1.088 | 4.01E−08 | 8.79E−07 |
| LOC283683 | −1.09 | 0.0007195 | 0.002001 |
| IRF7 | −1.091 | 8.61E−08 | 1.52E−06 |
| ECHDC3 | −1.091 | 1.03E−05 | 6.43E−05 |
| ABCC2 | −1.092 | 0.002229 | 0.005217 |
| ABI1 | −1.093 | 2.47E−09 | 1.24E−07 |
| ESF1 | −1.093 | 1.70E−05 | 9.44E−05 |
| CDK17 | −1.094 | 1.48E−08 | 4.38E−07 |
| PARG | −1.094 | 1.82E−08 | 5.14E−07 |
| ASB13 | −1.094 | 9.15E−08 | 1.60E−06 |
| RABGGTB | −1.094 | 2.27E−06 | 1.97E−05 |
| ZNF184 | −1.095 | 4.30E−07 | 5.37E−06 |
| UGCG | −1.095 | 2.48E−06 | 2.11E−05 |
| PPFIBP1 | −1.096 | 9.27E−08 | 1.60E−06 |
| FUOM | −1.097 | 2.60E−09 | 1.28E−07 |
| TOMM40L | −1.098 | 4.06E−09 | 1.75E−07 |
| OASL | −1.099 | 4.40E−06 | 3.34E−05 |
| ATE1 | −1.101 | 2.29E−08 | 6.09E−07 |
| ARRDC3 | −1.101 | 5.63E−07 | 6.58E−06 |
| BEND6 | −1.101 | 2.48E−05 | 0.0001276 |
| GSTO1 | −1.102 | 4.48E−08 | 9.62E−07 |
| HMGCS1 | −1.102 | 8.74E−08 | 1.54E−06 |
| MSX1 | −1.102 | 6.56E−06 | 4.55E−05 |
| PCED1B | −1.102 | 5.92E−05 | 0.0002565 |
| DDX21 | −1.103 | 1.11E−09 | 8.19E−08 |
| ZNF622 | −1.103 | 9.81E−09 | 3.27E−07 |
| NDUFA5 | −1.103 | 6.63E−05 | 0.0002813 |
| METTL18 | −1.104 | 2.87E−05 | 0.0001431 |
| NUP107 | −1.106 | 2.93E−09 | 1.40E−07 |
| AP1S2 | −1.106 | 1.81E−07 | 2.70E−06 |
| CENPE | −1.106 | 3.67E−06 | 2.90E−05 |
| PRIM1 | −1.107 | 8.23E−07 | 8.94E−06 |
| LINC01165 | −1.107 | 0.006718 | 0.01353 |
| CCPG1 | −1.108 | 7.79E−07 | 8.53E−06 |
| ZSCAN26 | −1.109 | 3.99E−06 | 3.09E−05 |
| MIR155HG | −1.109 | 0.0001358 | 0.0005036 |
| NEUROG3 | −1.111 | 2.50E−06 | 2.12E−05 |
| ITGB1 | −1.112 | 5.84E−10 | 5.40E−08 |
| ZNF138 | −1.112 | 4.88E−06 | 3.61E−05 |
| LLPH | −1.113 | 7.93E−09 | 2.82E−07 |
| BIK | −1.113 | 1.82E−08 | 5.14E−07 |
| BLZF1 | −1.113 | 7.21E−07 | 8.02E−06 |
| ZNF257 | −1.113 | 2.41E−06 | 2.07E−05 |
| CCDC6 | −1.114 | 7.57E−09 | 2.72E−07 |
| FAM188A | −1.115 | 6.55E−07 | 7.44E−06 |
| HMMR | −1.115 | 2.84E−06 | 2.37E−05 |
| MRPS30 | −1.117 | 1.97E−09 | 1.12E−07 |
| DNAJB4 | −1.117 | 8.77E−06 | 5.64E−05 |
| CCDC58 | −1.117 | 3.24E−05 | 0.0001573 |
| SNORA32 | −1.119 | 0.0006375 | 0.00181 |
| CCL28 | −1.12 | 1.04E−07 | 1.75E−06 |
| SERPINB10 | −1.12 | 1.23E−06 | 1.22E−05 |
| SNHG8 | −1.121 | 1.02E−06 | 1.06E−05 |
| EIF3C | −1.121 | 3.02E−05 | 0.0001489 |
| FAM173B | −1.122 | 2.11E−07 | 3.05E−06 |
| TMEM255A | −1.123 | 1.47E−05 | 8.49E−05 |
| GTPBP4 | −1.126 | 1.88E−09 | 1.09E−07 |
| MCM10 | −1.128 | 2.01E−09 | 1.13E−07 |
| EPSTI1 | −1.129 | 3.97E−06 | 3.08E−05 |
| TIGD3 | −1.13 | 0.0004915 | 0.001458 |
| CAPRIN2 | −1.131 | 1.86E−05 | 0.0001008 |
| SLA2 | −1.132 | 2.65E−05 | 0.0001346 |
| DDAH2 | −1.133 | 5.40E−08 | 1.10E−06 |
| MNS1 | −1.133 | 4.49E−06 | 3.39E−05 |
| MGAT4C | −1.133 | 0.01342 | 0.02462 |
| ARHGAP19 | −1.134 | 3.53E−09 | 1.57E−07 |
| PHYH | −1.134 | 1.99E−08 | 5.47E−07 |
| MRPL42 | −1.135 | 2.81E−06 | 2.34E−05 |
| PAOX | −1.135 | 1.56E−05 | 8.83E−05 |
| UPF2 | −1.137 | 4.88E−09 | 2.01E−07 |
| HSP90AA1 | −1.137 | 7.48E−07 | 8.27E−06 |
| NCAM2 | −1.138 | 5.44E−10 | 5.16E−08 |
| RP11-119F19.2 | −1.138 | 1.35E−05 | 7.91E−05 |
| TNKS2 | −1.14 | 1.54E−09 | 9.52E−08 |
| PGAM1 | −1.14 | 1.72E−09 | 1.03E−07 |
| ALDOC | −1.14 | 8.44E−06 | 5.48E−05 |
| THAP9 | −1.14 | 3.83E−05 | 0.0001811 |
| ASCC1 | −1.141 | 2.31E−08 | 6.09E−07 |
| HERC4 | −1.142 | 1.15E−09 | 8.27E−08 |
| RICTOR | −1.142 | 1.00E−07 | 1.71E−06 |
| PRTFDC1 | −1.142 | 6.36E−05 | 0.0002714 |
| LOC100506901 | −1.143 | 0.004126 | 0.008845 |
| TMEM191C | −1.143 | 0.01557 | 0.02805 |
| FRS2 | −1.144 | 3.34E−08 | 7.78E−07 |
| ZNF439 | −1.145 | 7.25E−08 | 1.34E−06 |
| NUF2 | −1.145 | 6.51E−06 | 4.53E−05 |
| ULK2 | −1.146 | 1.17E−06 | 1.18E−05 |
| DNAJC1 | −1.148 | 2.89E−09 | 1.40E−07 |
| ZNF33A | −1.15 | 8.53E−08 | 1.52E−06 |
| MUC19 | −1.15 | 0.004287 | 0.009145 |
| FRAT2 | −1.151 | 8.25E−10 | 6.83E−08 |
| ARL6 | −1.151 | 3.80E−06 | 2.97E−05 |
| PPP1R2P3 | −1.151 | 0.002065 | 0.004894 |
| C10orf76 | −1.152 | 5.12E−09 | 2.08E−07 |
| SNHG3 | −1.152 | 1.42E−08 | 4.28E−07 |
| LARP7 | −1.152 | 1.83E−05 | 9.98E−05 |
| CDHR1 | −1.153 | 0.001108 | 0.002876 |
| EGFR | −1.154 | 0.004619 | 0.00974 |
| ZNF267 | −1.155 | 2.52E−06 | 2.14E−05 |
| LINC01085 | −1.155 | 0.0005412 | 0.00158 |
| ADK | −1.157 | 2.91E−09 | 1.40E−07 |
| FAM105A | −1.159 | 2.19E−09 | 1.17E−07 |
| FAM84B | −1.16 | 6.89E−07 | 7.74E−06 |
| LOC102723616 | −1.16 | 0.001835 | 0.004433 |
| PPA1 | −1.161 | 3.87E−09 | 1.69E−07 |
| NUDCD1 | −1.161 | 1.72E−06 | 1.58E−05 |
| RAD1 | −1.162 | 1.51E−09 | 9.51E−08 |
| ZNF816 | −1.162 | 3.89E−07 | 4.97E−06 |
| FAM178A | −1.164 | 2.96E−09 | 1.40E−07 |
| CIITA | −1.165 | 8.76E−05 | 0.0003524 |
| WHAMMP2 | −1.166 | 4.44E−07 | 5.47E−06 |
| C5orf34 | −1.166 | 1.90E−06 | 1.71E−05 |
| PANK1 | −1.168 | 5.60E−08 | 1.12E−06 |
| LINC00843 | −1.168 | 5.44E−07 | 6.42E−06 |
| ZNF441 | −1.171 | 5.40E−07 | 6.40E−06 |
| FZD8 | −1.174 | 7.35E−05 | 0.0003059 |
| ANKRD46 | −1.177 | 3.10E−06 | 2.54E−05 |
| NXT2 | −1.177 | 3.20E−06 | 2.60E−05 |
| TUBA8 | −1.178 | 6.39E−06 | 4.47E−05 |
| GDI2 | −1.18 | 1.74E−09 | 1.03E−07 |
| ZMYND11 | −1.18 | 2.42E−09 | 1.23E−07 |
| ACBD5 | −1.181 | 8.59E−09 | 2.98E−07 |
| LEPREL2 | −1.181 | 0.0001328 | 0.0004945 |
| MAPK8 | −1.183 | 1.43E−08 | 4.28E−07 |
| C15orf38 | −1.184 | 5.83E−08 | 1.15E−06 |
| LINC00938 | −1.184 | 2.38E−07 | 3.36E−06 |
| SIRT1 | −1.185 | 4.83E−09 | 2.00E−07 |
| BLNK | −1.185 | 0.0003809 | 0.001178 |
| DUSP8 | −1.185 | 0.0005816 | 0.00168 |
| ZNF91 | −1.186 | 9.33E−08 | 1.61E−06 |
| ARHGAP22 | −1.186 | 0.0005596 | 0.001625 |
| MSRB3 | −1.187 | 2.98E−09 | 1.40E−07 |
| NELL2 | −1.187 | 8.34E−06 | 5.43E−05 |
| PPP3CB | −1.188 | 2.13E−09 | 1.17E−07 |
| EPC1 | −1.188 | 5.43E−09 | 2.17E−07 |
| FCGR2B | −1.188 | 0.0003221 | 0.001027 |
| ZNF675 | −1.19 | 2.05E−07 | 3.00E−06 |
| CHST3 | −1.191 | 3.35E−07 | 4.43E−06 |
| SLC35G1 | −1.191 | 2.26E−05 | 0.0001187 |
| SNORD5 | −1.191 | 0.0002453 | 0.0008186 |
| LOC339862 | −1.192 | 0.0001269 | 0.0004769 |
| SNORA8 | −1.193 | 0.0002001 | 0.0006908 |
| FRA10AC1 | −1.194 | 6.68E−08 | 1.26E−06 |
| OSCAR | −1.194 | 5.72E−07 | 6.66E−06 |
| SHISA7 | −1.194 | 4.79E−05 | 0.000216 |
| MPPED1 | −1.195 | 0.003465 | 0.007609 |
| RALGPS2 | −1.196 | 1.06E−07 | 1.78E−06 |
| WAPAL | −1.198 | 1.57E−09 | 9.68E−08 |
| UTF1 | −1.198 | 0.008533 | 0.01662 |
| SNORD22 | −1.199 | 9.19E−05 | 0.0003666 |
| RMRP | −1.199 | 0.001875 | 0.004514 |
| BUB3 | −1.203 | 3.35E−10 | 3.76E−08 |
| C8orf88 | −1.204 | 2.13E−07 | 3.07E−06 |
| DACH1 | −1.205 | 1.53E−09 | 9.52E−08 |
| CDC123 | −1.206 | 2.18E−09 | 1.17E−07 |
| ARL14EP | −1.209 | 8.34E−06 | 5.43E−05 |
| PAIP1 | −1.21 | 2.38E−09 | 1.22E−07 |
| DENND1B | −1.21 | 1.33E−07 | 2.14E−06 |
| RASSF9 | −1.21 | 0.000825 | 0.002248 |
| SDC2 | −1.212 | 2.51E−09 | 1.25E−07 |
| KAZALD1 | −1.212 | 5.90E−06 | 4.20E−05 |
| VEGFA | −1.215 | 2.63E−07 | 3.64E−06 |
| SECTM1 | −1.215 | 4.93E−06 | 3.65E−05 |
| KIF5B | −1.216 | 1.83E−10 | 2.76E−08 |
| GIN1 | −1.22 | 1.50E−05 | 8.58E−05 |
| MCMBP | −1.222 | 2.07E−10 | 3.00E−08 |
| RPP38 | −1.223 | 2.19E−08 | 5.87E−07 |
| ZNF724P | −1.225 | 5.08E−05 | 0.0002268 |
| SMNDC1 | −1.228 | 1.87E−09 | 1.09E−07 |
| AHNAK2 | −1.229 | 4.94E−06 | 3.65E−05 |
| ANKRD26P3 | −1.23 | 0.0002994 | 0.0009658 |
| SUMO1P3 | −1.23 | 0.0003051 | 0.0009816 |
| ZNF487 | −1.23 | 0.00046 | 0.001379 |
| NUTM2D | −1.23 | 0.01303 | 0.02403 |
| TEX30 | −1.231 | 3.48E−07 | 4.57E−06 |
| MMP11 | −1.231 | 0.0001617 | 0.0005802 |
| ZDHHC6 | −1.232 | 1.83E−09 | 1.08E−07 |
| ZWINT | −1.234 | 3.18E−10 | 3.70E−08 |
| FAM53B | −1.235 | 9.59E−09 | 3.23E−07 |
| FAIM | −1.235 | 1.13E−06 | 1.14E−05 |
| LRRC40 | −1.235 | 1.11E−05 | 6.81E−05 |
| FGL2 | −1.236 | 6.58E−06 | 4.56E−05 |
| CREM | −1.238 | 2.75E−07 | 3.76E−06 |
| HDHD3 | −1.24 | 2.40E−08 | 6.26E−07 |
| ZNF14 | −1.243 | 5.66E−07 | 6.62E−06 |
| NNT-AS1 | −1.244 | 1.11E−08 | 3.59E−07 |
| ITPR3 | −1.244 | 2.85E−08 | 6.97E−07 |
| ZNF432 | −1.245 | 9.04E−06 | 5.78E−05 |
| MAGEB2 | −1.246 | 2.22E−09 | 1.17E−07 |
| AGAP9 | −1.246 | 6.66E−06 | 4.59E−05 |
| ARTN | −1.246 | 1.03E−05 | 6.44E−05 |
| ZNF568 | −1.247 | 8.25E−06 | 5.39E−05 |
| PRDX3 | −1.249 | 3.54E−09 | 1.57E−07 |
| CES3 | −1.249 | 1.61E−07 | 2.45E−06 |
| P2RX7 | −1.252 | 6.69E−05 | 0.0002831 |
| SNORD44 | −1.253 | 0.01001 | 0.0191 |
| WBP1L | −1.254 | 4.39E−10 | 4.59E−08 |
| FAM175B | −1.254 | 9.03E−10 | 7.05E−08 |
| CEP55 | −1.255 | 2.57E−10 | 3.33E−08 |
| FAM160B1 | −1.255 | 9.76E−08 | 1.67E−06 |
| WBP5 | −1.255 | 3.65E−06 | 2.88E−05 |
| LZTS2 | −1.256 | 3.11E−10 | 3.68E−08 |
| KIAA1586 | −1.257 | 1.44E−06 | 1.38E−05 |
| ARL3 | −1.258 | 3.63E−08 | 8.26E−07 |
| CCT2 | −1.259 | 1.20E−08 | 3.79E−07 |
| IFIT3 | −1.259 | 1.99E−07 | 2.92E−06 |
| FAM60A | −1.26 | 1.20E−09 | 8.51E−08 |
| BNIP3 | −1.26 | 1.93E−08 | 5.34E−07 |
| AIMP1 | −1.26 | 4.92E−07 | 5.94E−06 |
| ELMOD2 | −1.26 | 3.77E−06 | 2.96E−05 |
| LPCAT1 | −1.261 | 6.46E−10 | 5.74E−08 |
| GLRX3 | −1.261 | 9.12E−09 | 3.12E−07 |
| ZNF737 | −1.261 | 9.87E−07 | 1.03E−05 |
| GXYLT1 | −1.263 | 2.85E−09 | 1.38E−07 |
| TARS | −1.264 | 3.06E−10 | 3.68E−08 |
| INPP5F | −1.265 | 3.15E−07 | 4.22E−06 |
| SFMBT2 | −1.266 | 8.11E−09 | 2.86E−07 |
| TBK1 | −1.267 | 2.04E−07 | 2.99E−06 |
| QRICH2 | −1.269 | 0.0002054 | 0.0007051 |
| LOC101060181 | −1.273 | 0.0005085 | 0.001499 |
| BMS1P6 | −1.274 | 0.001323 | 0.003339 |
| GRIA3 | −1.275 | 2.28E−06 | 1.98E−05 |
| OTUD6B | −1.277 | 2.66E−06 | 2.24E−05 |
| KCNS1 | −1.277 | 0.0005475 | 0.001596 |
| LOC100288152 | −1.279 | 1.37E−05 | 8.03E−05 |
| MRPS16 | −1.281 | 1.93E−09 | 1.10E−07 |
| HES4 | −1.282 | 0.0003976 | 0.00122 |
| FLJ37201 | −1.282 | 0.006494 | 0.01313 |
| SFXN4 | −1.285 | 2.31E−08 | 6.09E−07 |
| DCLRE1C | −1.286 | 4.90E−10 | 4.97E−08 |
| IDE | −1.29 | 1.18E−09 | 8.46E−08 |
| DCLRE1A | −1.29 | 1.25E−07 | 2.03E−06 |
| TLE6 | −1.291 | 7.99E−06 | 5.26E−05 |
| RP11-454P21.1 | −1.291 | 0.0003444 | 0.001088 |
| SUV39H2 | −1.296 | 1.45E−08 | 4.30E−07 |
| ZFAND4 | −1.297 | 6.16E−07 | 7.08E−06 |
| KITLG | −1.298 | 1.66E−08 | 4.76E−07 |
| ADAMTS14 | −1.298 | 0.002214 | 0.005183 |
| FAM45A | −1.301 | 4.40E−09 | 1.85E−07 |
| ALG10 | −1.301 | 2.46E−07 | 3.46E−06 |
| ZNF680 | −1.302 | 6.91E−07 | 7.75E−06 |
| CUL2 | −1.303 | 1.33E−09 | 9.04E−08 |
| ARHGAP12 | −1.303 | 7.63E−09 | 2.74E−07 |
| SLITRK5 | −1.304 | 3.74E−08 | 8.45E−07 |
| RAD54B | −1.305 | 1.11E−07 | 1.85E−06 |
| SEC14L2 | −1.307 | 4.77E−05 | 0.0002157 |
| LOC101927104 | −1.31 | 0.0001735 | 0.000614 |
| TM9SF3 | −1.311 | 2.17E−10 | 3.00E−08 |
| LOC101928913 | −1.311 | 0.0006498 | 0.001839 |
| SMC3 | −1.312 | 9.74E−11 | 2.04E−08 |
| HELLS | −1.313 | 1.99E−08 | 5.47E−07 |
| BMPR1A | −1.314 | 3.46E−07 | 4.56E−06 |
| MRPL36 | −1.315 | 6.23E−09 | 2.36E−07 |
| HLA-DPA1 | −1.316 | 0.0003633 | 0.001133 |
| RPP30 | −1.318 | 5.82E−09 | 2.27E−07 |
| ZNF503-AS2 | −1.318 | 2.25E−05 | 0.0001185 |
| MEIS3 | −1.319 | 0.0002859 | 0.0009283 |
| PRPF18 | −1.323 | 3.05E−09 | 1.42E−07 |
| SNORA24 | −1.324 | 0.001313 | 0.003319 |
| ULK4P2 | −1.324 | 0.003413 | 0.00751 |
| LRRC10B | −1.327 | 8.19E−05 | 0.0003335 |
| KCNK1 | −1.327 | 0.0003484 | 0.001097 |
| ZNF506 | −1.328 | 6.56E−09 | 2.44E−07 |
| FOXF1 | −1.328 | 1.55E−06 | 1.45E−05 |
| DNAJC9 | −1.33 | 8.61E−10 | 6.95E−08 |
| PCGF5 | −1.331 | 7.87E−08 | 1.43E−06 |
| LOC728407 | −1.331 | 0.0007165 | 0.001994 |
| ALG10B | −1.336 | 1.72E−06 | 1.58E−05 |
| PLEKHA8P1 | −1.337 | 1.10E−07 | 1.83E−06 |
| CLIC5 | −1.337 | 0.001485 | 0.003692 |
| MNAT1 | −1.341 | 7.93E−08 | 1.44E−06 |
| BCAT1 | −1.343 | 2.90E−09 | 1.40E−07 |
| ANAPC16 | −1.345 | 6.93E−09 | 2.56E−07 |
| RNLS | −1.348 | 8.82E−07 | 9.47E−06 |
| ENTPD1-AS1 | −1.348 | 5.33E−05 | 0.0002355 |
| CADM1 | −1.349 | 1.46E−06 | 1.39E−05 |
| ZNF92 | −1.351 | 1.37E−06 | 1.34E−05 |
| AP1S3 | −1.352 | 3.24E−10 | 3.73E−08 |
| HECTD2 | −1.352 | 7.68E−06 | 5.09E−05 |
| RP11-96H19.1 | −1.354 | 5.63E−06 | 4.05E−05 |
| SNORA25 | −1.354 | 0.00018 | 0.0006332 |
| IL31RA | −1.356 | 1.97E−06 | 1.75E−05 |
| LOC100507487 | −1.357 | 0.0001818 | 0.0006381 |
| USP6NL-IT1 | −1.357 | 0.0002521 | 0.0008374 |
| BTAF1 | −1.358 | 1.47E−07 | 2.28E−06 |
| EBF3 | −1.36 | 7.24E−09 | 2.63E−07 |
| C12orf29 | −1.36 | 1.77E−06 | 1.61E−05 |
| NKD2 | −1.361 | 4.41E−09 | 1.85E−07 |
| TMEM117 | −1.361 | 3.98E−07 | 5.07E−06 |
| LYSMD1 | −1.363 | 0.000107 | 0.0004145 |
| RPL22L1 | −1.364 | 4.29E−07 | 5.37E−06 |
| PXDC1 | −1.364 | 2.36E−05 | 0.0001225 |
| COMMD3 | −1.366 | 2.33E−07 | 3.31E−06 |
| GHITM | −1.367 | 1.42E−09 | 9.21E−08 |
| CEP290 | −1.37 | 9.25E−08 | 1.60E−06 |
| ACTR6 | −1.37 | 1.55E−06 | 1.45E−05 |
| TFEC | −1.372 | 5.18E−06 | 3.79E−05 |
| UBE2D1 | −1.374 | 2.26E−07 | 3.23E−06 |
| DEPDC4 | −1.374 | 7.00E−06 | 4.76E−05 |
| BLOC1S2 | −1.375 | 9.24E−08 | 1.60E−06 |
| LOC101928524 | −1.377 | 8.57E−05 | 0.0003465 |
| RP11-12A2.3 | −1.378 | 5.33E−05 | 0.0002355 |
| FSD1L | −1.385 | 5.97E−06 | 4.24E−05 |
| C2CD4C | −1.387 | 0.0001417 | 0.0005217 |
| DPCD | −1.388 | 7.48E−06 | 5.00E−05 |
| CES4A | −1.388 | 0.0008601 | 0.002324 |
| LCOR | −1.392 | 2.30E−08 | 6.09E−07 |
| KIAA1324 | −1.394 | 4.11E−05 | 0.0001912 |
| KIN | −1.396 | 5.60E−09 | 2.21E−07 |
| C1QTNF3 | −1.398 | 2.50E−06 | 2.12E−05 |
| BBIP1 | −1.399 | 1.63E−07 | 2.48E−06 |
| C2CD5 | −1.4 | 1.63E−09 | 9.93E−08 |
| TLX3 | −1.402 | 4.50E−06 | 3.40E−05 |
| LMBRD2 | −1.406 | 2.73E−07 | 3.75E−06 |
| LRRC17 | −1.407 | 4.20E−05 | 0.0001944 |
| TFAM | −1.409 | 5.27E−09 | 2.12E−07 |
| NFYB | −1.41 | 8.24E−07 | 8.94E−06 |
| NCOA4 | −1.411 | 5.32E−11 | 1.39E−08 |
| C10orf55 | −1.411 | 0.00014 | 0.0005165 |
| SNORD47 | −1.413 | 0.0001106 | 0.0004256 |
| ARL5B | −1.418 | 3.52E−07 | 4.62E−06 |
| TIMM23 | −1.419 | 3.47E−10 | 3.83E−08 |
| ZNF33B | −1.419 | 1.06E−08 | 3.46E−07 |
| PSTPIP2 | −1.42 | 7.97E−10 | 6.68E−08 |
| GOLT1B | −1.421 | 2.14E−08 | 5.77E−07 |
| PPP1R3F | −1.424 | 3.33E−06 | 2.68E−05 |
| NUDT13 | −1.424 | 4.02E−05 | 0.0001883 |
| OXCT1 | −1.426 | 8.30E−11 | 1.79E−08 |
| CAMK1D | −1.426 | 1.43E−08 | 4.28E−07 |
| RGS10 | −1.427 | 2.79E−09 | 1.36E−07 |
| RPL37 | −1.428 | 2.84E−08 | 6.96E−07 |
| DNAJC21 | −1.429 | 1.15E−10 | 2.20E−08 |
| METTL10 | −1.435 | 1.63E−09 | 9.93E−08 |
| CCDC7 | −1.437 | 2.07E−05 | 0.0001106 |
| NEURL1 | −1.441 | 4.80E−08 | 1.01E−06 |
| TMTC3 | −1.441 | 1.47E−07 | 2.28E−06 |
| JMJD1C | −1.442 | 4.41E−09 | 1.85E−07 |
| AMN1 | −1.442 | 1.71E−07 | 2.60E−06 |
| KRR1 | −1.443 | 1.94E−07 | 2.87E−06 |
| PTPRO | −1.448 | 1.32E−09 | 9.04E−08 |
| SNHG12 | −1.448 | 5.47E−07 | 6.45E−06 |
| OGDHL | −1.449 | 3.88E−05 | 0.0001828 |
| SLC38A2 | −1.45 | 4.39E−11 | 1.28E−08 |
| ANO6 | −1.45 | 2.16E−10 | 3.00E−08 |
| SAMD11 | −1.45 | 6.94E−05 | 0.0002918 |
| IL17RB | −1.453 | 2.88E−05 | 0.0001432 |
| CWF19L1 | −1.454 | 4.86E−10 | 4.97E−08 |
| IDI1 | −1.455 | 4.47E−07 | 5.50E−06 |
| ZNF85 | −1.455 | 6.05E−07 | 6.99E−06 |
| PTER | −1.463 | 1.34E−09 | 9.04E−08 |
| LRRC27 | −1.463 | 1.40E−06 | 1.35E−05 |
| CCDC177 | −1.465 | 0.002963 | 0.006665 |
| EXOC6 | −1.469 | 2.25E−09 | 1.17E−07 |
| SYCP2 | −1.47 | 0.0005772 | 0.001669 |
| ZNF726 | −1.473 | 7.62E−05 | 0.0003151 |
| DNA2 | −1.474 | 3.37E−09 | 1.53E−07 |
| SNORD104 | −1.475 | 0.0001337 | 0.0004976 |
| ANKRD26 | −1.476 | 3.46E−08 | 7.97E−07 |
| SHISA3 | −1.477 | 4.03E−07 | 5.10E−06 |
| ZNF438 | −1.479 | 7.15E−11 | 1.65E−08 |
| AGAP4 | −1.481 | 3.31E−06 | 2.67E−05 |
| VPS26A | −1.483 | 7.51E−11 | 1.70E−08 |
| CLCF1 | −1.485 | 0.0003625 | 0.001131 |
| RNF175 | −1.488 | 3.54E−08 | 8.11E−07 |
| GNG11 | −1.495 | 2.74E−05 | 0.0001375 |
| LINC00477 | −1.507 | 3.24E−06 | 2.62E−05 |
| HSPA14 | −1.509 | 1.31E−09 | 9.04E−08 |
| SAMD8 | −1.509 | 6.36E−08 | 1.22E−06 |
| DENND5B | −1.509 | 6.36E−08 | 1.22E−06 |
| TYSND1 | −1.51 | 1.50E−09 | 9.51E−08 |
| LAT | −1.511 | 2.81E−08 | 6.93E−07 |
| BCCIP | −1.515 | 2.18E−09 | 1.17E−07 |
| RAB18 | −1.515 | 5.78E−09 | 2.26E−07 |
| METTL20 | −1.521 | 5.51E−05 | 0.0002419 |
| FAM35A | −1.524 | 4.84E−09 | 2.00E−07 |
| ZNF518A | −1.524 | 9.48E−09 | 3.22E−07 |
| UNC80 | −1.524 | 9.00E−05 | 0.0003605 |
| CPNE8 | −1.529 | 8.01E−09 | 2.84E−07 |
| TRUB1 | −1.53 | 1.38E−09 | 9.05E−08 |
| CAPS2 | −1.53 | 0.00192 | 0.004596 |
| USMG5 | −1.533 | 1.77E−08 | 5.02E−07 |
| CEP70 | −1.534 | 1.46E−07 | 2.28E−06 |
| ZNF32 | −1.536 | 1.22E−08 | 3.81E−07 |
| FAM45B | −1.536 | 2.97E−08 | 7.16E−07 |
| IPMK | −1.538 | 8.45E−10 | 6.95E−08 |
| SNX16 | −1.538 | 5.15E−06 | 3.78E−05 |
| TM7SF3 | −1.541 | 1.81E−11 | 9.06E−09 |
| NEXN | −1.542 | 3.20E−06 | 2.60E−05 |
| THAP2 | −1.543 | 6.94E−07 | 7.77E−06 |
| PPHLN1 | −1.547 | 2.17E−10 | 3.00E−08 |
| LOC100130992 | −1.547 | 2.01E−06 | 1.78E−05 |
| ZNF503 | −1.551 | 9.43E−09 | 3.21E−07 |
| SNORA61 | −1.552 | 0.0001016 | 0.0003971 |
| SCAF11 | −1.558 | 1.06E−10 | 2.09E−08 |
| FAM204A | −1.558 | 1.61E−09 | 9.91E−08 |
| ZFR | −1.56 | 7.07E−08 | 1.32E−06 |
| BRIX1 | −1.561 | 6.94E−10 | 6.08E−08 |
| INPP5A | −1.561 | 1.02E−09 | 7.71E−08 |
| ZNF131 | −1.562 | 5.47E−10 | 5.16E−08 |
| DDX11 | −1.563 | 4.59E−10 | 4.76E−08 |
| HHEX | −1.563 | 1.48E−09 | 9.43E−08 |
| TUBB3 | −1.565 | 6.75E−05 | 0.0002849 |
| NOC3L | −1.566 | 2.35E−09 | 1.21E−07 |
| HTR7 | −1.566 | 4.32E−08 | 9.37E−07 |
| SLC4A11 | −1.566 | 1.01E−06 | 1.05E−05 |
| GPX8 | −1.568 | 1.28E−07 | 2.07E−06 |
| ADM | −1.568 | 0.0001005 | 0.000394 |
| SHOC2 | −1.571 | 6.19E−11 | 1.52E−08 |
| YAF2 | −1.584 | 6.23E−09 | 2.36E−07 |
| SNORD87 | −1.584 | 0.001157 | 0.002982 |
| ZNF98 | −1.589 | 3.96E−08 | 8.75E−07 |
| ASUN | −1.59 | 1.51E−10 | 2.50E−08 |
| FLJ37035 | −1.598 | 6.03E−05 | 0.0002607 |
| KIF11 | −1.603 | 2.80E−11 | 1.13E−08 |
| ATAD1 | −1.603 | 4.12E−09 | 1.77E−07 |
| SNORD76 | −1.606 | 0.0002649 | 0.0008721 |
| PARPBP | −1.607 | 8.32E−09 | 2.92E−07 |
| P4HA1 | −1.607 | 2.82E−08 | 6.93E−07 |
| SEPP1 | −1.607 | 0.0005001 | 0.001479 |
| LOC647859 | −1.613 | 0.0004781 | 0.001426 |
| ZNF25 | −1.614 | 1.00E−09 | 7.64E−08 |
| SLK | −1.614 | 1.51E−09 | 9.51E−08 |
| SEC61A2 | −1.614 | 4.50E−06 | 3.40E−05 |
| CHUK | −1.62 | 6.41E−10 | 5.73E−08 |
| ZNF485 | −1.622 | 9.39E−08 | 1.62E−06 |
| SLC16A9 | −1.623 | 1.59E−08 | 4.61E−07 |
| CASC5 | −1.626 | 2.15E−09 | 1.17E−07 |
| ZNF511 | −1.627 | 8.04E−11 | 1.77E−08 |
| ARNTL2 | −1.63 | 8.79E−10 | 6.98E−08 |
| SLC7A11 | −1.63 | 3.31E−09 | 1.50E−07 |
| CISD1 | −1.638 | 4.16E−08 | 9.08E−07 |
| PDSS1 | −1.642 | 2.29E−10 | 3.10E−08 |
| IFIT2 | −1.647 | 8.57E−08 | 1.52E−06 |
| NEGR1 | −1.647 | 4.58E−07 | 5.60E−06 |
| CHCHD1 | −1.651 | 1.44E−08 | 4.29E−07 |
| C1QA | −1.652 | 2.92E−05 | 0.0001453 |
| KLHL42 | −1.658 | 3.49E−11 | 1.18E−08 |
| CRABP1 | −1.663 | 8.64E−09 | 2.99E−07 |
| ZNF22 | −1.665 | 6.11E−09 | 2.34E−07 |
| RPS24 | −1.665 | 8.75E−08 | 1.54E−06 |
| DNM1L | −1.667 | 2.60E−11 | 1.13E−08 |
| C10orf32 | −1.667 | 7.29E−09 | 2.64E−07 |
| LOC728819 | −1.669 | 3.24E−05 | 0.0001574 |
| ARSG | −1.671 | 4.63E−07 | 5.65E−06 |
| OAT | −1.672 | 3.19E−10 | 3.70E−08 |
| SMAD9 | −1.676 | 0.001001 | 0.002644 |
| ABCC6P1 | −1.687 | 9.32E−05 | 0.0003709 |
| PAPSS2 | −1.688 | 1.84E−09 | 1.08E−07 |
| CUTC | −1.69 | 1.26E−09 | 8.85E−08 |
| NSMCE4A | −1.701 | 3.04E−08 | 7.28E−07 |
| CYP2B7P | −1.701 | 0.0004275 | 0.001295 |
| PCGF6 | −1.703 | 9.22E−10 | 7.16E−08 |
| LDHB | −1.706 | 3.18E−09 | 1.46E−07 |
| SLC2A13 | −1.707 | 7.10E−09 | 2.59E−07 |
| EPS8L2 | −1.707 | 0.0008933 | 0.002403 |
| MASTL | −1.709 | 6.25E−10 | 5.62E−08 |
| KIF20B | −1.709 | 1.01E−09 | 7.67E−08 |
| TRDMT1 | −1.711 | 5.52E−08 | 1.11E−06 |
| NKX6-3 | −1.713 | 1.14E−05 | 6.94E−05 |
| CMAS | −1.716 | 1.61E−11 | 8.78E−09 |
| TEX15 | −1.725 | 3.35E−08 | 7.78E−07 |
| RUFY2 | −1.73 | 5.57E−07 | 6.54E−06 |
| DDX50 | −1.733 | 6.19E−10 | 5.61E−08 |
| PEAR1 | −1.737 | 0.000203 | 0.0006989 |
| RNF208 | −1.747 | 5.62E−05 | 0.0002457 |
| KIAA1551 | −1.748 | 3.03E−11 | 1.13E−08 |
| RASSF8 | −1.754 | 2.95E−08 | 7.13E−07 |
| NCAM1 | −1.761 | 6.98E−09 | 2.57E−07 |
| SFR1 | −1.768 | 1.84E−08 | 5.16E−07 |
| YARS2 | −1.77 | 1.70E−10 | 2.64E−08 |
| SPEF2 | −1.772 | 1.52E−09 | 9.51E−08 |
| SLC16A6 | −1.773 | 0.0004402 | 0.001328 |
| STK38L | −1.774 | 1.55E−10 | 2.50E−08 |
| CTBP2 | −1.779 | 1.58E−11 | 8.78E−09 |
| RECQL | −1.785 | 1.44E−09 | 9.27E−08 |
| EGR1 | −1.785 | 0.004835 | 0.01013 |
| KCNS3 | −1.789 | 4.59E−05 | 0.0002092 |
| VIM | −1.791 | 3.99E−12 | 4.50E−09 |
| PTENP1 | −1.798 | 9.92E−05 | 0.0003899 |
| SOCS2 | −1.804 | 6.17E−07 | 7.08E−06 |
| PAWR | −1.805 | 8.45E−07 | 9.11E−06 |
| SNORA73A | −1.807 | 0.0004286 | 0.001298 |
| ATP5C1 | −1.81 | 8.96E−09 | 3.08E−07 |
| CTNNA3 | −1.811 | 0.001171 | 0.003014 |
| SIGLEC1 | −1.816 | 1.72E−10 | 2.64E−08 |
| PUS7L | −1.827 | 3.41E−08 | 7.89E−07 |
| KREMEN2 | −1.829 | 1.06E−06 | 1.09E−05 |
| ANK3 | −1.83 | 0.0001689 | 0.000601 |
| HOXB6 | −1.832 | 8.88E−06 | 5.69E−05 |
| DPY19L2 | −1.838 | 0.0004262 | 0.001292 |
| CD209 | −1.869 | 4.86E−11 | 1.30E−08 |
| EPHB4 | −1.875 | 9.95E−09 | 3.31E−07 |
| MRPS35 | −1.902 | 3.12E−11 | 1.13E−08 |
| VAT1L | −1.908 | 1.65E−09 | 9.98E−08 |
| LTK | −1.911 | 7.76E−10 | 6.63E−08 |
| SHISA8 | −1.914 | 1.84E−05 | 0.0001003 |
| C5orf28 | −1.919 | 4.65E−08 | 9.87E−07 |
| SLC12A7 | −1.921 | 6.53E−10 | 5.76E−08 |
| SUB1 | −1.922 | 5.89E−08 | 1.16E−06 |
| CCDC91 | −1.933 | 3.10E−10 | 3.68E−08 |
| MEETTL12 | −1.943 | 5.83E−06 | 4.16E−05 |
| ETNK1 | −1.946 | 1.13E−09 | 8.23E−08 |
| LYRM5 | −1.953 | 5.48E−08 | 1.11E−06 |
| PCDHGA12 | −1.981 | 4.42E−06 | 3.35E−05 |
| KRAS | −1.982 | 6.64E−12 | 5.31E−09 |
| RP11-996F15.2 | −1.984 | 8.81E−06 | 5.66E−05 |
| FAM35BP | −1.987 | 5.40E−06 | 3.91E−05 |
| KCNQ5 | −1.995 | 1.39E−09 | 9.05E−08 |
| GUCY1B3 | −1.997 | 8.16E−06 | 5.34E−05 |
| ACPP | −2.006 | 4.76E−12 | 4.50E−09 |
| IFITM1 | −2.008 | 6.20E−10 | 5.61E−08 |
| PLEKHO1 | −2.01 | 1.06E−10 | 2.09E−08 |
| LOC100506688 | −2.012 | 3.84E−09 | 1.68E−07 |
| SPRED1 | −2.012 | 7.01E−05 | 0.000294 |
| LOC100506374 | −2.023 | 3.91E−06 | 3.04E−05 |
| LRMP | −2.038 | 1.18E−11 | 8.44E−09 |
| SOX1 | −2.056 | 1.99E−05 | 0.000107 |
| LOC101927646 | −2.067 | 3.09E−05 | 0.000152 |
| TWF1 | −2.069 | 1.38E−08 | 4.18E−07 |
| SETD6 | −2.078 | 2.47E−06 | 2.10E−05 |
| AKR1C3 | −2.08 | 3.83E−08 | 8.55E−07 |
| IFI44 | −2.092 | 2.51E−08 | 6.41E−07 |
| LOC102724472 | −2.093 | 1.40E−05 | 8.18E−05 |
| IFIT5 | −2.099 | 2.11E−07 | 3.05E−06 |
| FCER2 | −2.106 | 1.89E−10 | 2.79E−08 |
| AASS | −2.116 | 3.29E−05 | 0.0001593 |
| MED21 | −2.118 | 3.31E−09 | 1.50E−07 |
| CDK1 | −2.135 | 5.80E−08 | 1.15E−06 |
| ERGIC2 | −2.139 | 1.68E−08 | 4.82E−07 |
| LOC101927799 | −2.144 | 7.45E−06 | 4.99E−05 |
| UGT3A2 | −2.153 | 2.25E−09 | 1.17E−07 |
| SOX5 | −2.164 | 1.22E−06 | 1.22E−05 |
| LOC284600 | −2.179 | 0.000259 | 0.0008566 |
| CDH2 | −2.197 | 3.00E−09 | 1.41E−07 |
| FGD4 | −2.208 | 9.99E−09 | 3.31E−07 |
| PYROXD1 | −2.213 | 2.34E−09 | 1.21E−07 |
| TGFBR3L | −2.215 | 1.87E−07 | 2.78E−06 |
| GLIS2 | −2.222 | 1.33E−06 | 1.30E−05 |
| MCF2L2 | −2.251 | 0.0009547 | 0.002543 |
| TTC33 | −2.259 | 1.34E−08 | 4.12E−07 |
| PCED1B-AS1 | −2.26 | 7.85E−09 | 2.80E−07 |
| GPC6 | −2.27 | 6.93E−06 | 4.73E−05 |
| ABCD2 | −2.272 | 1.10E−06 | 1.13E−05 |
| ZNF43 | −2.282 | 5.07E−10 | 4.99E−08 |
| OVOL2 | −2.284 | 3.63E−08 | 8.26E−07 |
| TRIM22 | −2.293 | 5.28E−10 | 5.16E−08 |
| MDGA2 | −2.307 | 8.94E−05 | 0.0003584 |
| FGFR1OP2 | −2.323 | 1.33E−09 | 9.04E−08 |
| TOM1L1 | −2.338 | 1.12E−08 | 3.59E−07 |
| LOC441666 | −2.342 | 1.84E−08 | 5.16E−07 |
| PCDHB14 | −2.385 | 6.65E−06 | 4.59E−05 |
| RPAP3 | −2.431 | 1.84E−11 | 9.06E−09 |
| GRTP1 | −2.438 | 1.19E−05 | 7.16E−05 |
| MX1 | −2.44 | 1.17E−10 | 2.20E−08 |
| RHBDL3 | −2.443 | 8.49E−05 | 0.0003439 |
| HMX2 | −2.444 | 2.46E−08 | 6.37E−07 |
| LGALS3BP | −2.457 | 1.37E−08 | 4.17E−07 |
| SHANK3 | −2.457 | 3.11E−08 | 7.40E−07 |
| DDIT4 | −2.473 | 3.03E−10 | 3.68E−08 |
| COL27A1 | −2.475 | 1.55E−10 | 2.50E−08 |
| LOC100506691 | −2.496 | 2.59E−08 | 6.59E−07 |
| CA3 | −2.517 | 3.58E−07 | 4.68E−06 |
| GUSBP5 | −2.549 | 3.12E−06 | 2.55E−05 |
| DAPK2 | −2.559 | 1.29E−08 | 4.01E−07 |
| PCSK5 | −2.565 | 1.04E−06 | 1.07E−05 |
| MT1G | −2.606 | 2.21E−09 | 1.17E−07 |
| GAS1 | −2.652 | 1.69E−09 | 1.02E−07 |
| AGAP7 | −2.657 | 8.75E−06 | 5.63E−05 |
| SNORA1 | −2.672 | 1.84E−06 | 1.66E−05 |
| ZNF829 | −2.701 | 9.61E−08 | 1.65E−06 |
| PCDHB13 | −2.716 | 2.73E−08 | 6.81E−07 |
| ZNF572 | −2.716 | 6.35E−08 | 1.22E−06 |
| RP11-53019.1 | −2.739 | 2.87E−08 | 6.99E−07 |
| IFIT1 | −2.744 | 5.39E−09 | 2.16E−07 |
| AKR1E2 | −2.746 | 3.10E−07 | 4.17E−06 |
| HSH2D | −2.747 | 1.49E−11 | 8.78E−09 |
| KIF26A | −2.785 | 8.35E−09 | 2.92E−07 |
| IRAK4 | −2.835 | 4.39E−12 | 4.50E−09 |
| ZNF320 | −2.849 | 3.65E−11 | 1.19E−08 |
| RGL3 | −2.85 | 7.06E−06 | 4.79E−05 |
| FIGN | −2.85 | 6.64E−05 | 0.0002819 |
| ELK1 | −2.872 | 1.62E−11 | 8.78E−09 |
| ZNF99 | −2.909 | 2.21E−07 | 3.15E−06 |
| LINC00461 | −2.95 | 4.88E−05 | 0.0002193 |
| LOC100996425 | −2.991 | 2.91E−06 | 2.42E−05 |
| HLA-DRA | −2.999 | 1.28E−10 | 2.36E−08 |
| HSD17B8 | −3.064 | 1.46E−07 | 2.28E−06 |
| CSMD3 | −3.101 | 0.0002328 | 0.000783 |
| C6orf223 | −3.102 | 3.67E−11 | 1.19E−08 |
| TLX1 | −3.134 | 1.93E−06 | 1.73E−05 |
| ALX1 | −3.176 | 3.41E−10 | 3.80E−08 |
| RP11-184I16.4 | −3.222 | 4.97E−12 | 4.50E−09 |
| C10orf10 | −3.28 | 4.27E−07 | 5.35E−06 |
| DYNC1I1 | −3.29 | 2.20E−06 | 1.92E−05 |
| NES | −3.346 | 7.38E−08 | 1.36E−06 |
| TNNI3 | −3.386 | 2.29E−08 | 6.09E−07 |
| PTEN | −3.416 | 5.45E−15 | 4.71E−11 |
| IFI44L | −3.425 | 1.52E−10 | 2.50E−08 |
| CPS1 | −3.432 | 1.72E−06 | 1.58E−05 |
| SV2A | −3.448 | 1.41E−07 | 2.23E−06 |
| CADPS2 | −3.448 | 1.42E−06 | 1.37E−05 |
| IFI27 | −3.451 | 2.20E−09 | 1.17E−07 |
| ZNF662 | −3.591 | 4.79E−08 | 1.01E−06 |
| MPPED2 | −3.635 | 1.31E−06 | 1.28E−05 |
| PVRL3 | −3.73 | 5.81E−07 | 6.75E−06 |
| SMAD1 | −3.734 | 7.07E−11 | 1.65E−08 |
| RTN1 | −3.897 | 1.38E−08 | 4.19E−07 |
| SLFN12 | −3.902 | 9.53E−09 | 3.22E−07 |
| HMX3 | −3.973 | 7.07E−09 | 2.59E−07 |
| AIF1L | −3.981 | 1.47E−07 | 2.29E−06 |
| TPTEP1 | −4.027 | 6.56E−09 | 2.44E−07 |
| YPEL3 | −4.027 | 1.10E−07 | 1.84E−06 |
| C3orf18 | −4.112 | 4.71E−08 | 9.97E−07 |
| INPP5J | −4.166 | 3.52E−08 | 8.11E−07 |
| PAX5 | −4.211 | 1.12E−09 | 8.20E−08 |
| TRIM72 | −4.298 | 2.11E−07 | 3.05E−06 |
| VENTX | −4.317 | 3.75E−05 | 0.0001781 |
| TMEM63C | −4.504 | 7.11E−08 | 1.33E−06 |
| HLA-F | −4.589 | 1.76E−07 | 2.64E−06 |
| PI15 | −4.746 | 3.23E−07 | 4.30E−06 |
| C1QC | −4.893 | 1.91E−07 | 2.83E−06 |
| UNC13D | −5.014 | 6.21E−13 | 1.36E−09 |
| SIDT1 | −5.08 | 5.71E−07 | 6.66E−06 |
| CXorf57 | −5.085 | 3.38E−11 | 1.18E−08 |
| PAK6 | −5.381 | 6.33E−09 | 2.37E−07 |
| AKR1C2 | −5.484 | 1.43E−12 | 1.95E−09 |
| IL17RC | −5.534 | 5.17E−06 | 3.79E−05 |
| MAGEA4 | −5.651 | 1.06E−08 | 3.47E−07 |
| LDOC1 | −5.769 | 1.72E−08 | 4.92E−07 |
| ZNF404 | −5.785 | 1.11E−07 | 1.85E−06 |
| MT1L | −5.912 | 1.71E−09 | 1.03E−07 |
| PKIA | −5.97 | 2.01E−09 | 1.13E−07 |
| ANKFN1 | −5.991 | 4.30E−08 | 9.34E−07 |
| LOC100653233 | −6.247 | 3.54E−10 | 3.83E−08 |
| COL4A5 | −6.267 | 7.20E−10 | 6.23E−08 |
| CNTN1 | −6.719 | 1.41E−10 | 2.46E−08 |
| GYPE | −7.245 | 1.33E−10 | 2.38E−08 |
| BEX5 | −7.505 | 6.82E−11 | 1.63E−08 |
| AKR1C1 | −7.68 | 1.34E−09 | 9.04E−08 |
| ADAMTS9 | −9.535 | 6.25E−11 | 1.52E−08 |

TABLE 6
Differentially expressed genes in MyD88-KO vs WT THP 1 cells

| Gene | Log(FC) | P Value | Adj P Value |
|---|---|---|---|
| IFI27 | 5.004 | 1.11E−11 | 1.11E−11 |
| PCSK5 | 4.205 | 3.27E−10 | 3.27E−10 |
| WIPF3 | 3.104 | 4.25E−06 | 4.25E−06 |
| ANKRD19P | 2.834 | 2.16E−05 | 2.16E−05 |
| TAL1 | 2.767 | 4.27E−05 | 4.27E−05 |
| PGM5P3-AS1 | 2.718 | 1.36E−05 | 1.36E−05 |
| PTPN13 | 2.55 | 0.0008288 | 0.0008288 |
| SPON1 | 2.501 | 0.001977 | 0.001977 |
| GABRA4 | 2.304 | 1.40E−05 | 1.40E−05 |
| BDNF | 2.274 | 0.0004115 | 0.0004115 |
| RAB38 | 2.123 | 8.81E−05 | 8.81E−05 |
| TIAM2 | 2.086 | 0.000142 | 0.000142 |
| SCRN1 | 2.032 | 2.09E−09 | 2.09E−09 |
| LOC729737 | 2.032 | 0.0004831 | 0.0004831 |
| DLEU7-AS1 | 1.968 | 8.62E−08 | 8.62E−08 |
| LONRF2 | 1.941 | 0.0004953 | 0.0004953 |
| RNU4-1 | 1.925 | 0.1368 | 0.1368 |
| IFI44L | 1.92 | 1.05E−05 | 1.05E−05 |
| CCDC120 | 1.855 | 1.38E−06 | 1.38E−06 |
| PPP1R36 | 1.809 | 0.01049 | 0.01049 |
| RFK | 1.777 | 3.99E−10 | 3.99E−10 |
| DMD | 1.758 | 0.0002615 | 0.0002615 |
| HFE | 1.741 | 0.0001211 | 0.0001211 |
| FIGN | 1.724 | 2.02E−05 | 2.02E−05 |
| HOXB13 | 1.722 | 7.85E−05 | 7.85E−05 |
| HOXB6 | 1.719 | 4.02E−07 | 4.02E−07 |
| ROBO4 | 1.719 | 0.008999 | 0.008999 |
| BTNL9 | 1.718 | 0.0003396 | 0.0003396 |
| SLC6A9 | 1.701 | 2.01E−05 | 2.01E−05 |
| LOC645166 | 1.672 | 0.0002332 | 0.0002332 |
| MTMR7 | 1.631 | 0.001032 | 0.001032 |
| LSR | 1.624 | 0.0005328 | 0.0005328 |
| APOLD1 | 1.613 | 2.65E−07 | 2.65E−07 |
| RUFY4 | 1.61 | 4.21E−05 | 4.21E−05 |
| TFPI | 1.588 | 0.0005722 | 0.0005722 |
| VAT1L | 1.56 | 7.06E−09 | 7.06E−09 |
| ZNF827 | 1.551 | 0.0004303 | 0.0004303 |
| RN7SK | 1.544 | 0.1016 | 0.1016 |
| EPSTI1 | 1.538 | 7.25E−06 | 7.25E−06 |
| FIRRE | 1.536 | 0.011 | 0.011 |
| LAMA2 | 1.525 | 4.37E−07 | 4.37E−07 |
| NAPSA | 1.509 | 0.001484 | 0.001484 |
| DLL1 | 1.487 | 1.16E−06 | 1.16E−06 |
| CCND1 | 1.487 | 5.05E−06 | 5.05E−06 |
| SARM1 | 1.459 | 8.01E−05 | 8.01E−05 |
| HFM1 | 1.422 | 0.008283 | 0.008283 |
| LINC01356 | 1.417 | 0.002198 | 0.002198 |
| SLC47A1 | 1.403 | 0.005899 | 0.005899 |
| LAMB2P1 | 1.395 | 0.006539 | 0.006539 |
| PLBD1 | 1.385 | 7.13E−06 | 7.13E−06 |
| VENTX | 1.375 | 2.64E−05 | 2.64E−05 |
| RMRP | 1.375 | 0.1139 | 0.1139 |
| RNA18S5 | 1.358 | 0.05323 | 0.05323 |
| PPP1R32 | 1.355 | 0.0003128 | 0.0003128 |
| ZNF462 | 1.345 | 1.66E−05 | 1.66E−05 |
| PGM5 | 1.344 | 0.00357 | 0.00357 |
| SIGLEC1 | 1.333 | 1.77E−06 | 1.77E−06 |
| CDC42BPA | 1.331 | 3.97E−07 | 3.97E−07 |
| PCDHGA8 | 1.327 | 0.001193 | 0.001193 |
| MX1 | 1.299 | 2.09E−08 | 2.09E−08 |
| MIR2052HG | 1.298 | 0.005069 | 0.005069 |
| GABRD | 1.293 | 0.000356 | 0.000356 |
| SLC22A23 | 1.287 | 0.002727 | 0.002727 |
| LOC101929415 | 1.277 | 0.004777 | 0.004777 |
| A2M | 1.269 | 5.47E−07 | 5.47E−07 |
| ASIC3 | 1.269 | 0.002579 | 0.002579 |
| NPR2 | 1.25 | 0.001993 | 0.001993 |
| IFI44 | 1.248 | 6.10E−05 | 6.10E−05 |
| TMEM63C | 1.247 | 0.0001438 | 0.0001438 |
| IFI6 | 1.245 | 9.85E−08 | 9.85E−08 |
| SARDH | 1.245 | 0.0006221 | 0.0006221 |
| CNKSR1 | 1.245 | 0.002316 | 0.002316 |
| CNIH3 | 1.22 | 0.001653 | 0.001653 |
| POLR2J3 | 1.204 | 0.0002499 | 0.0002499 |
| CAPN6 | 1.186 | 0.0001565 | 0.0001565 |
| GMPR | 1.182 | 0.004771 | 0.004771 |
| ASPRV1 | 1.174 | 0.009129 | 0.009129 |
| ZNF630 | 1.172 | 0.0004312 | 0.0004312 |
| TRIM22 | 1.165 | 2.43E−06 | 2.43E−06 |
| COL4A5 | 1.164 | 6.41E−06 | 6.41E−06 |
| GNG7 | 1.163 | 7.36E−07 | 7.36E−07 |
| DMC1 | 1.154 | 0.002323 | 0.002323 |
| NEXN | 1.152 | 1.51E−05 | 1.51E−05 |
| LARP6 | 1.149 | 0.002797 | 0.002797 |
| SLC35F3 | 1.132 | 0.01293 | 0.01293 |
| HYDIN2 | 1.126 | 0.0006369 | 0.0006369 |
| HOXB7 | 1.125 | 0.001243 | 0.001243 |
| LPAR6 | 1.125 | 0.01413 | 0.01413 |
| PRAC2 | 1.12 | 0.001021 | 0.001021 |
| PFN2 | 1.119 | 0.0007115 | 0.0007115 |
| MAEL | 1.117 | 0.008775 | 0.008775 |
| RNU4-2 | 1.115 | 0.4748 | 0.4748 |
| VLDLR-AS1 | 1.114 | 1.74E−05 | 1.74E−05 |
| CFAP70 | 1.111 | 0.003466 | 0.003466 |
| SYT11 | 1.109 | 0.0001933 | 0.0001933 |
| FAM171A1 | 1.102 | 0.002471 | 0.002471 |
| GRTP1 | 1.101 | 0.001091 | 0.001091 |
| LIPN | 1.098 | 0.001701 | 0.001701 |
| OAS3 | 1.094 | 6.61E−09 | 6.61E−09 |
| LOC101929592 | 1.094 | 2.20E−07 | 2.20E−07 |
| PI15 | 1.083 | 0.0008236 | 0.0008236 |
| FLJ32255 | 1.081 | 1.26E−08 | 1.26E−08 |
| CNTN1 | 1.081 | 0.0008388 | 0.0008388 |
| LINC01169 | 1.077 | 0.00114 | 0.00114 |
| KIF9-AS1 | 1.075 | 0.005 | 0.005 |
| OVOL2 | 1.071 | 9.45E−06 | 9.45E−06 |
| TBC1D3P1-DHX40P1 | 1.071 | 0.05772 | 0.05772 |
| PNMA2 | 1.07 | 0.002867 | 0.002867 |
| MSR1 | 1.066 | 0.002334 | 0.002334 |
| NBPF9 | 1.057 | 0.0003286 | 0.0003286 |
| CGN | 1.055 | 0.0002051 | 0.0002051 |
| MEGF6 | 1.046 | 6.84E−05 | 6.84E−05 |
| MIR7641-2 | 1.044 | 0.228 | 0.228 |
| DLX3 | 1.043 | 1.30E−05 | 1.30E−05 |
| OTUD1 | 1.043 | 1.24E−05 | 1.24E−05 |
| RPS6KA2 | 1.043 | 3.93E−05 | 3.93E−05 |
| PCDHB12 | 1.037 | 0.02716 | 0.02716 |
| TMEM254-AS1 | 1.036 | 0.01687 | 0.01687 |
| OAS1 | 1.035 | 6.85E−07 | 6.85E−07 |
| RNA28S5 | 1.032 | 0.004217 | 0.004217 |
| SYTL3 | 1.031 | 0.0006695 | 0.0006695 |
| KIRREL | 1.028 | 0.001776 | 0.001776 |
| PMEPA1 | 1.025 | 0.003844 | 0.003844 |
| SCN8A | 1.023 | 0.004269 | 0.004269 |
| GNAL | 1.023 | 0.008918 | 0.008918 |
| LINC01270 | 1.022 | 0.02285 | 0.02285 |
| ITGA2 | 1.021 | 0.0001891 | 0.0001891 |
| OAS2 | 1.02 | 1.49E−08 | 1.49E−08 |
| ARFGEF3 | 1.018 | 9.72E−05 | 9.72E−05 |
| CBS | 1.017 | 0.0361 | 0.0361 |
| ABLIM2 | 1.014 | 0.0004158 | 0.0004158 |
| TMEM221 | 1.014 | 0.004114 | 0.004114 |
| CBWD3 | 1.003 | 0.01803 | 0.01803 |
| ADAMTS9 | −9.776 | 1.87E−11 | 1.87E−11 |
| MFNG | −9.746 | 2.79E−10 | 2.79E−10 |
| ELOVL3 | −7.35 | 6.64E−10 | 6.64E−10 |
| ALPK3 | −7.059 | 9.97E−11 | 9.97E−11 |
| ZNF37A | −6.359 | 1.36E−07 | 1.36E−07 |
| ANKFN1 | −6.277 | 1.04E−08 | 1.04E−08 |
| KCNK1 | −6.189 | 9.78E−09 | 9.78E−09 |
| ZNF662 | −5.935 | 8.93E−08 | 8.93E−08 |
| CNTNAP4 | −5.859 | 2.19E−08 | 2.19E−08 |
| MT1L | −5.789 | 2.30E−08 | 2.30E−08 |
| VWA1 | −5.707 | 2.57E−08 | 2.57E−08 |
| LINC01446 | −5.707 | 2.57E−08 | 2.57E−08 |
| MAGED1 | −5.675 | 1.02E−07 | 1.02E−07 |
| ZNF829 | −5.528 | 1.11E−07 | 1.11E−07 |
| LINC00654 | −5.197 | 5.10E−07 | 5.10E−07 |
| LDOC1 | −5.067 | 2.59E−06 | 2.59E−06 |
| PLEKHB1 | −4.983 | 8.69E−07 | 8.69E−07 |
| LRRC8E | −4.878 | 4.18E−06 | 4.18E−06 |
| MT1G | −4.865 | 2.65E−09 | 2.65E−09 |
| GNG11 | −4.777 | 7.80E−06 | 7.80E−06 |
| LOC102724661 | −4.651 | 2.00E−06 | 2.00E−06 |
| CADPS2 | −4.61 | 3.66E−07 | 3.66E−07 |
| SLFN12 | −4.431 | 1.22E−07 | 1.22E−07 |
| MAGEB2 | −4.408 | 1.26E−11 | 1.26E−11 |
| PINLYP | −4.318 | 1.74E−05 | 1.74E−05 |
| ZNF536 | −4.274 | 5.55E−07 | 5.55E−07 |
| UNCX | −4.224 | 0.0001804 | 0.0001804 |
| CCRL2 | −4.14 | 9.39E−07 | 9.39E−07 |
| AKR1C2 | −4.135 | 1.37E−10 | 1.37E−10 |
| IL12RB2 | −3.951 | 3.12E−11 | 3.12E−11 |
| RGL3 | −3.872 | 0.000209 | 0.000209 |
| PNCK | −3.862 | 7.77E−07 | 7.77E−07 |
| NUDT16P1 | −3.804 | 1.24E−07 | 1.24E−07 |
| TOM1L1 | −3.708 | 5.28E−09 | 5.28E−09 |
| DYNC1I1 | −3.708 | 3.07E−06 | 3.07E−06 |
| CD93 | −3.526 | 8.36E−14 | 8.36E−14 |
| CKM | −3.523 | 5.39E−08 | 5.39E−08 |
| PRSS30P | −3.46 | 1.78E−05 | 1.78E−05 |
| SIX3 | −3.422 | 1.66E−06 | 1.66E−06 |
| POU4F2 | −2.998 | 2.72E−08 | 2.72E−08 |
| EPS8L2 | −2.856 | 0.0001462 | 0.0001462 |
| CNTN5 | −2.81 | 4.00E−05 | 4.00E−05 |
| PCDHGA12 | −2.714 | 3.30E−06 | 3.30E−06 |
| CYP27A1 | −2.714 | 4.08E−05 | 4.08E−05 |
| PAK6 | −2.654 | 1.78E−07 | 1.78E−07 |
| NES | −2.632 | 2.24E−06 | 2.24E−06 |
| KDELC1 | −2.596 | 1.10E−06 | 1.10E−06 |
| FRMD4A | −2.58 | 1.89E−08 | 1.89E−08 |
| C2 | −2.565 | 5.89E−08 | 5.89E−08 |
| RGS16 | −2.557 | 6.88E−10 | 6.88E−10 |
| LIPG | −2.403 | 1.39E−06 | 1.39E−06 |
| IL17RC | −2.371 | 2.27E−06 | 2.27E−06 |
| SELM | −2.35 | 1.67E−05 | 1.67E−05 |
| SMO | −2.339 | 1.28E−05 | 1.28E−05 |
| SPRED1 | −2.318 | 3.01E−05 | 3.01E−05 |
| N4BP3 | −2.318 | 3.45E−05 | 3.45E−05 |
| C1orf186 | −2.239 | 1.27E−07 | 1.27E−07 |
| TEX41 | −2.209 | 7.61E−06 | 7.61E−06 |
| CTSW | −2.209 | 3.98E−05 | 3.98E−05 |
| FAM46C | −2.2 | 2.28E−05 | 2.28E−05 |
| TPTEP1 | −2.163 | 4.09E−07 | 4.09E−07 |
| CKAP4 | −2.139 | 1.82E−10 | 1.82E−10 |
| FAM163A | −2.094 | 7.18E−07 | 7.18E−07 |
| HSPA1A | −2.065 | 1.67E−09 | 1.67E−09 |
| SV2A | −2.055 | 6.05E−06 | 6.05E−06 |
| ST8SIA6 | −2.048 | 0.0001081 | 0.0001081 |
| NCAM1 | −2.045 | 4.82E−09 | 4.82E−09 |
| PAQR5 | −2.031 | 8.70E−05 | 8.70E−05 |
| NAV3 | −2.001 | 7.01E−10 | 7.01E−10 |
| GPX8 | −1.992 | 3.38E−07 | 3.38E−07 |
| PDE11A | −1.933 | 0.0003522 | 0.0003522 |
| ZNF568 | −1.915 | 2.09E−05 | 2.09E−05 |
| SPAG16 | −1.902 | 0.001127 | 0.001127 |
| BHLHE22 | −1.899 | 2.10E−06 | 2.10E−06 |
| AJAP1 | −1.898 | 4.40E−06 | 4.40E−06 |
| PSMD5-AS1 | −1.87 | 2.87E−05 | 2.87E−05 |
| SERPINA1 | −1.869 | 5.74E−05 | 5.74E−05 |
| HTRA3 | −1.818 | 0.000177 | 0.000177 |
| CARD11 | −1.797 | 2.56E−06 | 2.56E−06 |
| GPR35 | −1.791 | 3.60E−07 | 3.60E−07 |
| SLAMF1 | −1.777 | 0.0001023 | 0.0001023 |
| ALX1 | −1.76 | 1.71E−08 | 1.71E−08 |
| SULF2 | −1.755 | 2.12E−07 | 2.12E−07 |
| KCNK10 | −1.751 | 1.07E−05 | 1.07E−05 |
| ANKLE1 | −1.748 | 1.64E−08 | 1.64E−08 |
| PTENP1 | −1.723 | 9.81E−06 | 9.81E−06 |
| AHNAK2 | −1.722 | 2.06E−07 | 2.06E−07 |
| BCAM | −1.712 | 2.71E−06 | 2.71E−06 |
| G0S2 | −1.688 | 5.62E−07 | 5.62E−07 |
| LINC01088 | −1.68 | 1.06E−06 | 1.06E−06 |
| ANXA8L1 | −1.667 | 0.001985 | 0.001985 |
| CAMKV | −1.663 | 0.0001651 | 0.0001651 |
| DEFB1 | −1.642 | 4.35E−07 | 4.35E−07 |
| TESC | −1.63 | 3.06E−06 | 3.06E−06 |
| SIGLEC6 | −1.619 | 5.01E−06 | 5.01E−06 |
| TSPAN15 | −1.613 | 1.38E−05 | 1.38E−05 |
| MYO7A | −1.61 | 0.001237 | 0.001237 |
| LPAR5 | −1.595 | 7.84E−07 | 7.84E−07 |
| ETNK2 | −1.566 | 0.0002664 | 0.0002664 |
| PAX5 | −1.546 | 4.30E−08 | 4.30E−08 |
| HMCN1 | −1.529 | 0.0009012 | 0.0009012 |
| MS4A3 | −1.522 | 3.51E−08 | 3.51E−08 |
| SEMG1 | −1.515 | 2.32E−05 | 2.32E−05 |
| CEBPE | −1.503 | 1.72E−06 | 1.72E−06 |
| POLR2J2 | −1.491 | 0.002487 | 0.002487 |
| DPEP1 | −1.486 | 5.62E−05 | 5.62E−05 |
| AKR1C1 | −1.483 | 1.00E−05 | 1.00E−05 |
| AKR1C3 | −1.482 | 1.24E−05 | 1.24E−05 |
| FCAMR | −1.476 | 0.001059 | 0.001059 |
| PLD3 | −1.47 | 1.83E−10 | 1.83E−10 |
| SEPT5-GP1BB | −1.461 | 0.001132 | 0.001132 |
| FBXO15 | −1.45 | 9.74E−05 | 9.74E−05 |
| DNAH8 | −1.445 | 0.0006908 | 0.0006908 |
| TSPEAR-AS2 | −1.433 | 0.01173 | 0.01173 |
| SAMD14 | −1.426 | 0.002199 | 0.002199 |
| FBLN1 | −1.425 | 3.15E−05 | 3.15E−05 |
| NUDT11 | −1.4 | 0.001135 | 0.001135 |
| FUZ | −1.398 | 0.01073 | 0.01073 |
| GALR2 | −1.397 | 1.04E−05 | 1.04E−05 |
| PCGF2 | −1.387 | 9.30E−06 | 9.30E−06 |
| TLR9 | −1.373 | 0.001235 | 0.001235 |
| TUBA8 | −1.35 | 3.29E−05 | 3.29E−05 |
| CD52 | −1.348 | 4.36E−05 | 4.36E−05 |
| CFP | −1.342 | 6.28E−07 | 6.28E−07 |
| ARRDC4 | −1.339 | 2.64E−07 | 2.64E−07 |
| CIITA | −1.335 | 0.0003352 | 0.0003352 |
| PRAM1 | −1.33 | 6.08E−07 | 6.08E−07 |
| C6orf223 | −1.325 | 9.93E−08 | 9.93E−08 |
| FAM135B | −1.321 | 0.0001931 | 0.0001931 |
| C3AR1 | −1.311 | 0.008129 | 0.008129 |
| CXCL8 | −1.308 | 0.0001412 | 0.0001412 |
| AGK | −1.3 | 1.06E−07 | 1.06E−07 |
| SYN3 | −1.299 | 0.0009512 | 0.0009512 |
| DRC7 | −1.293 | 0.0009396 | 0.0009396 |
| SIGLEC10 | −1.289 | 2.16E−05 | 2.16E−05 |
| LRRC25 | −1.287 | 0.0003791 | 0.0003791 |
| APOC1 | −1.282 | 1.50E−07 | 1.50E−07 |
| TSPEAR-AS1 | −1.277 | 9.93E−05 | 9.93E−05 |
| GAS6-AS2 | −1.269 | 0.005078 | 0.005078 |
| PLTP | −1.267 | 1.21E−08 | 1.21E−08 |
| FEV | −1.263 | 0.005341 | 0.005341 |
| PLEKHA6 | −1.256 | 0.0002187 | 0.0002187 |
| SPOCK1 | −1.25 | 0.0007964 | 0.0007964 |
| TIFAB | −1.249 | 4.52E−08 | 4.52E−08 |
| HLA-DRA | −1.246 | 7.79E−06 | 7.79E−06 |
| FAM150A | −1.245 | 0.0003636 | 0.0003636 |
| TIMP3 | −1.238 | 1.17E−09 | 1.17E−09 |
| SLAMF9 | −1.237 | 0.001371 | 0.001371 |
| FAM46A | −1.235 | 2.28E−09 | 2.28E−09 |
| DLL4 | −1.23 | 0.001169 | 0.001169 |
| ANXA8 | −1.228 | 5.13E−05 | 5.13E−05 |
| PTAFR | −1.223 | 0.0005927 | 0.0005927 |
| SOX18 | −1.221 | 0.002136 | 0.002136 |
| ARHGAP22 | −1.219 | 0.001064 | 0.001064 |
| OLIG1 | −1.218 | 9.42E−06 | 9.42E−06 |
| PCDH7 | −1.217 | 1.77E−05 | 1.77E−05 |
| S100A8 | −1.216 | 1.20E−05 | 1.20E−05 |
| TNFSF4 | −1.214 | 0.007379 | 0.007379 |
| EHD2 | −1.211 | 9.63E−07 | 9.63E−07 |
| NFE2 | −1.209 | 5.99E−08 | 5.99E−08 |
| SPRY4 | −1.202 | 0.0001361 | 0.0001361 |
| MIP | −1.196 | 0.002865 | 0.002865 |
| PTPN14 | −1.186 | 7.50E−06 | 7.50E−06 |
| TEX30 | −1.183 | 4.62E−07 | 4.62E−07 |
| DCHS1 | −1.181 | 9.73E−06 | 9.73E−06 |
| TMEM37 | −1.179 | 0.0001138 | 0.0001138 |
| NID1 | −1.178 | 4.70E−09 | 4.70E−09 |
| SCIMP | −1.173 | 0.0002844 | 0.0002844 |
| TMEM261 | −1.171 | 7.89E−07 | 7.89E−07 |
| ATP1B1 | −1.167 | 3.26E−06 | 3.26E−06 |
| IRS2 | −1.165 | 4.88E−09 | 4.88E−09 |
| SLC2A10 | −1.164 | 0.001733 | 0.001733 |
| EGR3 | −1.164 | 0.005139 | 0.005139 |
| TMIGD3 | −1.161 | 0.002304 | 0.002304 |
| PTEN | −1.159 | 4.70E−09 | 4.70E−09 |
| RGS20 | −1.158 | 0.001548 | 0.001548 |
| ARHGAP44 | −1.155 | 1.16E−05 | 1.16E−05 |
| ABI3 | −1.155 | 0.01236 | 0.01236 |
| ANXA6 | −1.15 | 2.27E−09 | 2.27E−09 |
| CDHR1 | −1.143 | 0.000761 | 0.000761 |
| STMN3 | −1.141 | 1.42E−06 | 1.42E−06 |
| ZNF358 | −1.14 | 0.001154 | 0.001154 |
| CTDSPL | −1.134 | 2.32E−07 | 2.32E−07 |
| EGR4 | −1.132 | 0.001224 | 0.001224 |
| ACPP | −1.125 | 5.08E−09 | 5.08E−09 |
| S100A9 | −1.121 | 1.27E−07 | 1.27E−07 |
| MARCKS | −1.121 | 1.49E−07 | 1.49E−07 |
| SYNGR3 | −1.12 | 0.01674 | 0.01674 |
| PGR | −1.119 | 2.33E−05 | 2.33E−05 |
| EFEMP2 | −1.115 | 6.47E−06 | 6.47E−06 |
| SGSH | −1.114 | 0.0141 | 0.0141 |
| NCF1B | −1.112 | 1.14E−05 | 1.14E−05 |
| PEAR1 | −1.111 | 0.001386 | 0.001386 |
| RASL11A | −1.108 | 0.0002425 | 0.0002425 |
| HOXC10 | −1.095 | 0.02029 | 0.02029 |
| ZRANB3 | −1.09 | 6.35E−06 | 6.35E−06 |
| B3GNT9 | −1.088 | 0.0001692 | 0.0001692 |
| HSD11B1 | −1.079 | 4.12E−08 | 4.12E−08 |
| OLIG2 | −1.078 | 1.21E−05 | 1.21E−05 |
| MYO7B | −1.074 | 0.005325 | 0.005325 |
| ATP2B3 | −1.067 | 0.001907 | 0.001907 |
| GGT1 | −1.058 | 4.40E−08 | 4.40E−08 |
| LYNX1 | −1.049 | 0.006992 | 0.006992 |
| PTCRA | −1.045 | 0.003054 | 0.003054 |
| LOC101929771 | −1.044 | 0.006373 | 0.006373 |
| LOC102467222 | −1.043 | 0.02166 | 0.02166 |
| FMO4 | −1.041 | 4.20E−05 | 4.20E−05 |
| RAB7B | −1.038 | 7.48E−08 | 7.48E−08 |
| WNT7B | −1.032 | 1.12E−07 | 1.12E−07 |
| UNC5C | −1.032 | 0.000101 | 0.000101 |
| THBS1 | −1.03 | 0.003111 | 0.003111 |
| OAF | −1.026 | 6.79E−09 | 6.79E−09 |
| CLIC5 | −1.026 | 0.003755 | 0.003755 |
| ROPN1L | −1.025 | 0.004848 | 0.004848 |
| EPPK1 | −1.023 | 0.00705 | 0.00705 |
| MPZ | −1.019 | 3.10E−05 | 3.10E−05 |
| HIST1H1C | −1.017 | 0.000323 | 0.000323 |
| TUBB3 | −1.016 | 0.001204 | 0.001204 |
| KIF26A | −1.014 | 0.0005932 | 0.0005932 |
| DZIP1 | −1.013 | 4.23E−06 | 4.23E−06 |
| PPAPDC3 | −1.008 | 0.0002983 | 0.0002983 |
| DCANP1 | −1.004 | 1.36E−06 | 1.36E−06 |

TABLE 7
Differentially expressed genes in IRAK1-KO,
IRAK4-KO, and IRAK1/4-dKO vs WT THP1 cells

| IRAK1-KO (- MyD88KO) | IRAK4-KO (- MyD88KO) | IRAK1/4-dKO (- MyD88KO) |
|---|---|---|
| vs WT | vs WT | vs WT |
| SLC2A10 | FOXF1 | PLS3 |
| DMTN | LOC339862 | HSBP1L1 |
| LOC101926931 | LYSMD1 | CDC42BPA |
| RLTPR | MSRB3 | PPP2R3A |
| ATP10A | PPP1R3F | GNG12 |
| IRAK1 | SNORA24 | PENK |
| CNTN4 | SNORD22 | STC2 |
| UST | SNORD76 | DNAJC22 |
| RNF165 | ZNF320 | SH3D19 |
| PLEKHG5 | ZNF572 | GABRD |
| ITGB2-AS1 | PCDHGA12 | RAVER2 |
| NCF1C | IFIT1 | ARHGEF26 |
| ANKRD37 | SEPP1 | APBB2 |
| MARCH9 | TRIM22 | BAI2 |
| LY9 | RNF175 | DPEP1 |
| NGEF | EGR1 | LGI2 |
| TBX15 | SETD6 | PIP5K1A |
| SNORD116-4 | TTC33 | SOX18 |
| RP5-1057J7.6 | SNORA73A | BMP4 |
| HIVEP1 | IFITM1 | TMC4 |
| NTN1 | FAM35BP | KCNIP3 |
| TBX1 | IFIT5 | CCNO |
| DLEU7-AS1 | PYROXD1 | ULK4P2 |
| KIAA1147 | MED21 | FAM57A |
| THBS1 | FGFR1OP2 | SCARA3 |
| CDO1 | RP11-996F15.2 | CERS1 |
| MEGF6 | LOC441666 | BEND5 |
| NCF1 | ERGIC2 | HAPLN3 |
| TG | LDHB | DNMBP |
| DLL1 | PAWR | KIAA1522 |
| GJC1 | CDK1 | PIR |
| IPW | KCNS1 | GTF3C5 |
| ZFP92 | IFIT2 | ECHDC3 |
| IL4I1 | SIGLEC1 | ICAM1 |
| RAB33A | TWF1 | RAB3A |
| ADAMTS4 | STK38L | SDHA |
| MYLIP | PTPRO | C9orf116 |
| WNT10B | RP11-12A2.3 | MKI67 |
| RIMKLA | CUTC | LMTK3 |
| TTC30A | RUFY2 | MCM10 |
| FHL3 | ZNF485 | ADM2 |
| CD300A | ATP5C1 | HSD17B14 |
| AP000330.8 | RECQL | LOC102724002 |
| C17orf96 | ETNK1 | KCNH2 |
| CAND2 | MEIS3 | PRAME |
| TNFAIP3 | OAT | KIAA1279 |
| NMUR1 | BMI1 | LOC101929974 |
| PTK7 | DDX50 | ARHGAP21 |
| FOXF1 | LOC728819 | SULT2B1 |
| LOC339862 | UNC80 | ADCY6 |
| LYSMD1 | FLJ37035 | GBF1 |
| MSRB3 | SUB1 | TUBGCP2 |
| PPP1R3F | LOC728407 | CD19 |
| SNORA24 | CHCHD1 | SPIRE2 |
| SNORD22 | LOC100130992 | SH3PXD2B |
| SNORD76 | PDSS1 | AADAT |
| ANXA4 | NSMCE4A | FOXC1 |
| APLN | CCDC91 | ZBED3 |
| C1QL4 | LOC101928524 | TSKU |
| CCDC102A | C5orf28 | NRARP |
| CHDH | PPHLN1 | AMIGO2 |
| EGLN3 | KIAA1551 | PCBD1 |
| ENO2 | RNLS | TCAM1P |
| FADS2 | NEXN | FAM57B |
| HK2 | TRDMT1 | LOC101929021 |
| HSPA7 | SDC2 | PCK2 |
| IL17RB | RPS24 | CCDC127 |
| KLF2 | CWF19L1 | MGMT |
| LAMC3 | CASC5 | C6orf147 |
| LINC00263 | HHEX | TGFB1I1 |
| LOC101928092 | RASSF8 | DPP4 |
| LOC102724908 | TIMM23 | ALX4 |
| MIR210HG | BMS1P6 | TACC2 |
| NRTN | HSPA14 | WLS |
| PFKFB4 | ZNF25 | PACSIN3 |
| SCD | CEP70 | NAPA-AS1 |
| SLC4A3 | ZNF131 | LINC00999 |
| SLC6A8 | MUC19 | BAIAP3 |
| ZNF215 | LRRC17 | ANKH |
| ZNF395 | SNORD44 | VDAC2 |
| ADM | ZFR | TAF3 |
| ALDOC | CCDC7 | ECHS1 |
| BNIP3 | ZNF98 | EFNA3 |
| CA3 | SLC2A13 | CORO6 |
| CD209 | CPNE8 | TCTN2 |
| CSMD3 | RPL37 | HPS1 |
| DDIT4 | DNA2 | DNAJB12 |
| FCGR2B | NEGR1 | SPIB |
| FCGR2C | ZNF32 | SLC29A3 |
| FIGN | FAM45B | PHGDH |
| GLIS2 | ZNF33B | PTMS |
| SHISA8 | CHUK | TMEM91 |
| SOX5 | PCGF6 | SEC24C |
| TBC1D30 | METTL10 | HGD |
| TLE6 | ZNF22 | MSS51 |
| TLX1 | IPMK | TEAD3 |
| VAT1L | PTER | LARP4B |
| VEGFA | IFIT3 | ZCCHC24 |
| | SLC7A11 | ARHGEF16 |
| | GHITM | FOXO6 |
| | ATAD1 | FBXO18 |
| | BCCIP | PPRC1 |
| | CRIP2 | CCDC78 |
| | CADM1 | RHBDL1 |
| | USMG5 | SPINT1 |
| | KIF20B | STON2 |
| | ZFAND4 | NUDT8 |
| | C10orf55 | NEBL |
| | SLC38A2 | EPAS1 |
| | ANKRD26P3 | FAM178A |
| | DNAJC9 | PI4K2A |
| | MRPL36 | SNED1 |
| | NELL2 | STAM |
| | ENTPD1-AS1 | PLOD2 |
| | SLK | TM7SF2 |
| | TIGD3 | FGD1 |
| | HPS6 | PCDHB6 |
| | RP11-454P21.1 | CCNB3 |
| | ARID2 | MCMDC2 |
| | PARPBP | EDRF1 |
| | DNAJC21 | NRM |
| | GPER1 | PPIF |
| | ARHGAP12 | ATF5 |
| | BBIP1 | BRD9 |
| | BMPR1A | B3GALT4 |
| | KIN | NET1 |
| | WBP1L | TMEM169 |
| | TRUB1 | RPH3AL |
| | SNHG19 | DROSHA |
| | ZNF506 | RBMS2 |
| | ANKRD26 | POLR3A |
| | SFXN4 | TRIP13 |
| | EPC1 | LPHN1 |
| | HTR7 | ZMIZ1 |
| | YAF2 | RP11-344B5.2 |
| | FBXL15 | HNRNPF |
| | RGS10 | HSD17B7P2 |
| | RPL36A | SLIT1 |
| | BRIX1 | LOC648987 |
| | COMMD3 | ACTR1A |
| | SHOC2 | CCT5 |
| | C5orf34 | S1PR5 |
| | CEP55 | WDFY4 |
| | TOMM40L | GRK5 |
| | PRPF18 | SREBF1 |
| | MAP3K8 | STXBP5L |
| | PYROXD2 | VARS2 |
| | SLC35G1 | GAB2 |
| | AMN1 | ZFP36L1 |
| | CREM | CPNE7 |
| | BIK | HSPA2 |
| | ZNF439 | NAT8L |
| | GDI2 | DOK4 |
| | LOC101060181 | IGF1R |
| | ALG10 | KHK |
| | RPL41 | KIRREL2 |
| | EXOSC1 | COMTD1 |
| | FAIM | ZNF488 |
| | GLRX3 | CASC10 |
| | FAM35A | QRICH2 |
| | LMBRD2 | RBM17 |
| | CDC123 | LSS |
| | LOC102724168 | PAX8 |
| | BUB3 | PKN3 |
| | NCOA4 | HELLS |
| | VPS26A | FN3K |
| | RP11-134O21.1 | CDC42EP1 |
| | NOC3L | NFIX |
| | SUMO1P3 | BMS1 |
| | EXOC6 | STOX1 |
| | UPF2 | SEMA4B |
| | VIM-AS1 | SUPV3L1 |
| | FAM173B | FAM198A |
| | RPP30 | RCOR2 |
| | FAM160B1 | PDCD4 |
| | ZNF432 | PPAP2A |
| | ZNF487 | DDX12P |
| | LINC00843 | ACY3 |
| | BMS1P4 | TSPAN10 |
| | CUL2 | NUP155 |
| | MNAT1 | RIBC2 |
| | BLOC1S2 | PLCXD1 |
| | ANAPC16 | TP53INP2 |
| | AVPI1 | GOLPH3 |
| | METRN | WDR70 |
| | WHAMMP2 | BTRC |
| | FRAT2 | SVIL |
| | RAB18 | DTX4 |
| | CCL28 | DUSP5 |
| | LCOR | ECE1 |
| | DACH1 | MRPL43 |
| | ARL3 | SKP2 |
| | SOX4 | SNHG3 |
| | TMEM117 | MYRF |
| | GAL | ZFYVE27 |
| | NFYB | H2AFY2 |
| | ADK | NOLC1 |
| | PRDX3 | SLC25A28 |
| | NUP107 | WNK2 |
| | WAPAL | NDUFS6 |
| | DCLRE1C | SLC30A3 |
| | SUV39H2 | LOC642846 |
| | ARL5B | NEURL1B |
| | FAR2 | ARMC4 |
| | HLA-DPA1 | SFXN3 |
| | ZNF37BP | VASH2 |
| | RP11-119F19.2 | NOTUM |
| | AGAP6 | VCL |
| | ABI1 | LOC100506901 |
| | TNKS2 | CSTF2T |
| | ACBD5 | PRICKLE1 |
| | DNAJC1 | ZNF132 |
| | EIF3C | GPR153 |
| | NCKAP5L | ZMYND11 |
| | GSTO1 | DGCR5 |
| | SMC3 | OXER1 |
| | PPOX | VTI1A |
| | TBK1 | PCDHGC3 |
| | NSUN6 | MORC4 |
| | ZNF816 | EIF3A |
| | ADM | PDCD11 |
| | ALDOC | SLC38A1 |
| | BNIP3 | MC1R |
| | CA3 | CBS |
| | CD209 | AC002454.1 |
| | CSMD3 | PDCD6 |
| | DDIT4 | B4GALNT1 |
| | FCGR2B | RPP38 |
| | FCGR2C | SUGT1P1 |
| | FIGN | NEDD4L |
| | GLIS2 | PROCA1 |
| | SHISA8 | SH3PXD2A |
| | SOX5 | JAG2 |
| | TBC1D30 | FGFR3 |
| | TLE6 | TMEM191B |
| | TLX1 | SEPHS1 |
| | VAT1L | LOC100505761 |
| | VEGFA | C21orf58 |
| | AASS | FADS1 |
| | ABCC6P1 | TET1 |
| | ABCD2 | GBGT1 |
| | ADAMTS14 | KAT6B |
| | AGAP4 | KIFC2 |
| | AGAP9 | TAF5 |
| | AIFM2 | CASP7 |
| | AKR1C1 | PELI3 |
| | AKR1E2 | PP7080 |
| | ANK3 | ATN1 |
| | ANKRD33B | KIAA1598 |
| | ANO6 | FAM115A |
| | APBB1IP | NKX3-2 |
| | ARHGAP19 | CHCHD10 |
| | ARNTL2 | GPSM1 |
| | ARSG | LRP4 |
| | ARTN | CDT1 |
| | ASCC1 | EIF4EBP2 |
| | ASUN | AASS |
| | BAG3 | ABCC6P1 |
| | BEND4 | ABCD2 |
| | BEX5 | ADAMTS14 |
| | BLNK | AGAP4 |
| | BTAF1 | AGAP9 |
| | C10orf10 | AIFM2 |
| | C10orf12 | AKR1C1 |
| | C10orf76 | AKR1E2 |
| | C10orf91 | ANK3 |
| | C15orf38 | ANKRD33B |
| | C2CD4C | ANO6 |
| | C2CD5 | APBB1IP |
| | C3orf18 | ARHGAP19 |
| | CALHM2 | ARNTL2 |
| | CAMK1D | ARSG |
| | CAPRIN2 | ARTN |
| | CDH2 | ASCC1 |
| | CDHR1 | ASUN |
| | CEL | BAG3 |
| | CEP72 | BEND4 |
| | CES3 | BEX5 |
| | CES4A | BLNK |
| | CHST3 | BTAF1 |
| | CLIC5 | C10orf10 |
| | CMAS | C10orf12 |
| | CNNM2 | C10orf76 |
| | CNTN1 | C10orf91 |
| | COL27A1 | C15orf38 |
| | COL4A5 | C2CD4C |
| | CPS1 | C2CD5 |
| | CRABP1 | C3orf18 |
| | CTBP2 | CALHM2 |
| | CUEDC2 | CAMK1D |
| | CXorf57 | CAPRIN2 |
| | DAPK2 | CDH2 |
| | DDAH2 | CDHR1 |
| | DDX11 | CEL |
| | DDX11-AS1 | CEP72 |
| | DENND5B | CES3 |
| | DHTKD1 | CES4A |
| | DNM1L | CHST3 |
| | DPCD | CLIC5 |
| | DPY19L2 | CMAS |
| | DUSP5P1 | CNNM2 |
| | EBF3 | CNTN1 |
| | EFEMP2 | COL27A1 |
| | EGFR | COL4A5 |
| | ELK1 | CPS1 |
| | EPHB4 | CRABP1 |
| | FAM155B | CTBP2 |
| | FAM175B | CUEDC2 |
| | FAM21C | CXorf57 |
| | FAM45A | DAPK2 |
| | FAM53B | DDAH2 |
| | FAM84B | DDX11 |
| | FCER2 | DDX11-AS1 |
| | FGD4 | DENND5B |
| | FOXL2 | DHTKD1 |
| | FZD8 | DNM1L |
| | GAS1 | DPCD |
| | GPC6 | DPY19L2 |
| | GRIA3 | DUSP5P1 |
| | GUCY1B3 | EBF3 |
| | GYPE | EFEMP2 |
| | HDHD3 | EGFR |
| | HES4 | ELK1 |
| | HIF1AN | EPHB4 |
| | HMGCS1 | FAM155B |
| | HMX2 | FAM175B |
| | HMX3 | FAM21C |
| | HOXB13 | FAM45A |
| | HOXB6 | FAM53B |
| | HSD17B8 | FAM84B |
| | HSH2D | FCER2 |
| | IDE | FGD4 |
| | INPP5A | FOXL2 |
| | INPP5F | FZD8 |
| | INPP5J | GAS1 |
| | IPO8 | GPC6 |
| | IRAK4 | GRIA3 |
| | ITPR2 | GUCY1B3 |
| | ITPR3 | GYPE |
| | JMJD1C | HDHD3 |
| | KAZALD1 | HES4 |
| | KCNQ5 | HIF1AN |
| | KCNS3 | HMGCS1 |
| | KIAA1324 | HMX2 |
| | KIF11 | HMX3 |
| | KLHL42 | HOXB13 |
| | KLLN | HOXB6 |
| | KRAS | HSD17B8 |
| | KREMEN2 | HSH2D |
| | LAT | IDE |
| | LDB1 | INPP5A |
| | LGALS3BP | INPP5F |
| | LINC00477 | INPP5J |
| | LIPE | IPO8 |
| | LOC100288152 | IRAK4 |
| | LOC100506688 | ITPR2 |
| | LOC100506691 | ITPR3 |
| | LOC100507487 | JMJD1C |
| | LOC101927104 | KAZALD1 |
| | LOC101927799 | KCNQ5 |
| | LOC101928913 | KCNS3 |
| | LOC102723337 | KIAA1324 |
| | LOC102724472 | KIF11 |
| | LPCAT1 | KLHL42 |
| | LRMP | KLLN |
| | LRRC10B | KRAS |
| | LRRC20 | KREMEN2 |
| | LRRC27 | LAT |
| | LTK | LDB1 |
| | LY6G5C | LGALS3BP |
| | LZTS2 | LINC00477 |
| | MAP2K6 | LIPE |
| | MASTL | LOC100288152 |
| | MCIDAS | LOC100506688 |
| | MCMBP | LOC100506691 |
| | MDGA2 | LOC100507487 |
| | METTL12 | LOC101927104 |
| | METTL20 | LOC101927799 |
| | MMS19 | LOC101928913 |
| | MPPED2 | LOC102723337 |
| | MRPS16 | LOC102724472 |
| | MRPS35 | LPCAT1 |
| | MSX1 | LRMP |
| | MX1 | LRRC10B |
| | NDUFB8 | LRRC20 |
| | NEURL1 | LRRC27 |
| | NEUROG3 | LTK |
| | NFKB2 | LY6G5C |
| | NIM1K | LZTS2 |
| | NKD2 | MAP2K6 |
| | NLGN2 | MASTL |
| | NNT | MCIDAS |
| | NNT-AS1 | MCMBP |
| | NT5DC2 | MDGA2 |
| | NUDT13 | METTL12 |
| | OGDHL | METTL20 |
| | OVOL2 | MMS19 |
| | OXCT1 | MPPED2 |
| | P2RX7 | MRPS16 |
| | P4HA1 | MRPS35 |
| | PABPC1L | MSX1 |
| | PANK1 | MX1 |
| | PAOX | NDUFB8 |
| | PAPD7 | NEURL1 |
| | PAPSS2 | NEUROG3 |
| | PCDHB13 | NFKB2 |
| | PCDHB14 | NIM1K |
| | PCED1B | NKD2 |
| | PCED1B-AS1 | NLGN2 |
| | PCSK5 | NNT |
| | PDLIM1 | NNT-AS1 |
| | PEAR1 | NT5DC2 |
| | PFKP | NUDT13 |
| | PGAM1 | OGDHL |
| | PGAM4 | OVOL2 |
| | PI15 | OXCT1 |
| | PITX1 | P2RX7 |
| | PLEKHA8P1 | P4HA1 |
| | PLEKHO1 | PABPC1L |
| | PPFIBP1 | PANK1 |
| | PPP2R2D | PAOX |
| | PPP3CB | PAPD7 |
| | PRTFDC1 | PAPSS2 |
| | PSTPIP2 | PCDHB13 |
| | PTENP1 | PCDHB14 |
| | PVRL3 | PCED1B |
| | PWWP2B | PCED1B-AS1 |
| | PXDC1 | PCSK5 |
| | RNF208 | PDLIM1 |
| | ROBO1 | PEAR1 |
| | RP11-184I16.4 | PFKP |
| | RP11-53O19.1 | PGAM1 |
| | RP11-96H19.1 | PGAM4 |
| | RPAP3 | PI15 |
| | RTN1 | PITX1 |
| | SAMD11 | PLEKHA8P1 |
| | SAMD8 | PLEKHO1 |
| | SCAF11 | PPFIBP1 |
| | SCD5 | PPP2R2D |
| | SDHAP3 | PPP3CB |
| | SEC14L2 | PRTFDC1 |
| | SEC31B | PSTPIP2 |
| | SEC61A2 | PTENP1 |
| | SECTM1 | PVRL3 |
| | SFMBT2 | PWWP2B |
| | SFXN2 | PXDC1 |
| | SHANK3 | RNF208 |
| | SHISA3 | ROBO1 |
| | SHISA7 | RP11-184I16.4 |
| | SIDT1 | RP11-53O19.1 |
| | SKIDA1 | RP11-96H19.1 |
| | SLA2 | RPAP3 |
| | SLC12A7 | RTN1 |
| | SLC16A6 | SAMD11 |
| | SLC16A9 | SAMD8 |
| | SLC4A11 | SCAF11 |
| | SLITRK5 | SCD5 |
| | SMAD1 | SDHAP3 |
| | SNHG12 | SEC14L2 |
| | SNORA61 | SEC31B |
| | SNORD104 | SEC61A2 |
| | SOX1 | SECTM1 |
| | SPEF2 | SFMBT2 |
| | SRRM3 | SFXN2 |
| | SUFU | SHANK3 |
| | TARS | SHISA3 |
| | TCF7 | SHISA7 |
| | TEX15 | SIDT1 |
| | TGFBR3L | SKIDA1 |
| | THBD | SLA2 |
| | TIAL1 | SLC12A7 |
| | TLX3 | SLC16A6 |
| | TM7SF3 | SLC16A9 |
| | TMEM180 | SLC4A11 |
| | TMEM191C | SLITRK5 |
| | TMEM255A | SMAD1 |
| | TMEM63C | SNHG12 |
| | TRIM72 | SNORA61 |
| | TUBB3 | SNORD104 |
| | TYSND1 | SOX1 |
| | UGT3A2 | SPEF2 |
| | ULK2 | SRRM3 |
| | UNC13D | SUFU |
| | USP54 | TARS |
| | VENTX | TCF7 |
| | VIM | TEX15 |
| | XPNPEP1 | TGFBR3L |
| | YARS2 | THBD |
| | YPEL3 | TIAL1 |
| | ZDHHC16 | TLX3 |
| | ZDHHC6 | TM7SF3 |
| | ZNF43 | TMEM180 |
| | ZNF438 | TMEM191C |
| | ZNF503 | TMEM255A |
| | ZNF503-AS2 | TMEM63C |
| | ZNF511 | TRIM72 |
| | ZNF574 | TUBB3 |
| | ZNF622 | TYSND1 |
| | ZNF726 | UGT3A2 |
| | ZNF99 | ULK2 |
| | ZWINT | UNC13D |
| | | USP54 |
| | | VENTX |
| | | VIM |
| | | XPNPEP1 |
| | | YARS2 |
| | | YPEL3 |
| | | ZDHHC16 |
| | | ZDHHC6 |
| | | ZNF43 |
| | | ZNF438 |
| | | ZNF503 |
| | | ZNF503-AS2 |
| | | ZNF511 |
| | | ZNF574 |
| | | ZNF622 |
| | | ZNF726 |
| | | ZNF99 |
| | | ZWINT |
| | | ADM |
| | | ALDOC |
| | | BNIP3 |
| | | CA3 |
| | | CD209 |
| | | CSMD3 |
| | | DDIT4 |
| | | FCGR2B |
| | | FCGR2C |
| | | FIGN |
| | | GLIS2 |
| | | SHISA8 |
| | | SOX5 |
| | | TBC1D30 |
| | | TLE6 |
| | | TLX1 |
| | | VAT1L |
| | | VEGFA |
| | | ANXA4 |
| | | APLN |
| | | C1QL4 |
| | | CCDC102A |
| | | CHDH |
| | | EGLN3 |
| | | ENO2 |
| | | FADS2 |
| | | HK2 |
| | | HSPA7 |
| | | IL17RB |
| | | KLF2 |
| | | LAMC3 |
| | | LINC00263 |
| | | LOC101928092 |
| | | LOC102724908 |
| | | MIR210HG |
| | | NRTN |
| | | PFKFB4 |
| | | SCD |
| | | SLC4A3 |
| | | SLC6A8 |
| | | ZNF215 |
| | | ZNF395 |

TABLE 8
IRAK1 proximity analysis

| | | Abundance | | | | |
|---|---|---|---|---|---|---|
| | Gene | Ratio P-Value: | | | # Unique | |
| Accession | Symbol | (DOX)/(NO DOX) | Coverage % | # Peptides | Peptides | # AAs |
| Q9UNX3 | RPL26L1 | 1E−17 | 45 | 12 | 1 | 145 |
| Q9BZI7 | UPF3B | 1E−17 | 10 | 5 | 5 | 483 |
| Q5F1R6 | DNAJC21 | 1E−17 | 6 | 2 | 2 | 531 |
| O15156 | ZBTB7B | 1E−17 | 10 | 2 | 2 | 539 |
| P62745 | RHOB | 1E−17 | 17 | 2 | 1 | 196 |
| Q9NRX1 | PNO1 | 1E−17 | 13 | 2 | 2 | 252 |
| P62316 | SNRPD2 | 1E−17 | 17 | 2 | 2 | 118 |
| Q9HCK8 | CHD8 | 1E−17 | 2 | 3 | 3 | 2581 |
| P12931 | SRC | 1E−17 | 13 | 6 | 5 | 536 |
| Q12874 | SF3A3 | 1E−17 | 4 | 2 | 2 | 501 |
| O15294 | OGT | 1E−17 | 4 | 2 | 2 | 1046 |
| Q03252 | LMNB2 | 1E−17 | 7 | 5 | 1 | 620 |
| Q9H0G5 | NSRP1 | 1E−17 | 5 | 2 | 2 | 558 |
| Q14213 | EBI3 | 1E−17 | 8 | 2 | 2 | 229 |
| P06400 | RB1 | 1E−17 | 5 | 2 | 2 | 928 |
| P52209 | PGD | 1E−17 | 8 | 3 | 3 | 483 |
| Q8IY21 | DDX60 | 1E−17 | 2 | 2 | 2 | 1712 |
| Q86TB9 | PATL1 | 1E−17 | 4 | 2 | 2 | 770 |
| Q13325 | IFIT5 | 1E−17 | 6 | 2 | 2 | 482 |
| O15533 | TAPBP | 1E−17 | 7 | 3 | 3 | 448 |
| A6NC98 | CCDC88B | 1E−17 | 2 | 2 | 2 | 1476 |
| Q14847 | LASP1 | 1E−17 | 10 | 2 | 2 | 261 |
| P32456 | GBP2 | 1E−17 | 5 | 2 | 1 | 591 |
| Q9Y6W5 | WASF2 | 1E−17 | 8 | 3 | 3 | 498 |
| P34897 | SHMT2 | 1E−17 | 4 | 2 | 2 | 504 |
| P01859 | IGHG2 | 1E−17 | 6 | 2 | 2 | 326 |
| Q96C10 | DHX58 | 1E−17 | 7 | 3 | 3 | 678 |
| O43432 | EIF4G3 | 1E−17 | 2 | 3 | 1 | 1585 |
| P43304 | GPD2 | 1E−17 | 3 | 2 | 2 | 727 |
| O76031 | CLPX | 1E−17 | 4 | 2 | 2 | 633 |
| Q01201 | RELB | 1E−17 | 10 | 3 | 3 | 579 |
| Q8WXG1 | RSAD2 | 1E−17 | 6 | 2 | 2 | 361 |
| P67936 | TPM4 | 1E−17 | 20 | 8 | 1 | 248 |
| P09913 | IFIT2 | 1E−17 | 15 | 5 | 5 | 472 |
| Q16851 | UGP2 | 1E−17 | 7 | 3 | 3 | 508 |
| Q96PP9 | GBP4 | 1E−17 | 5 | 2 | 2 | 640 |
| P12273 | PIP | 1E−17 | 26 | 3 | 3 | 146 |
| Q05209 | PTPN12 | 1E−17 | 6 | 4 | 4 | 780 |
| P35637 | FUS | 5.46331E−08 | 8 | 2 | 2 | 526 |
| Q8IYM9 | TRIM22 | 1.92275E−07 | 10 | 3 | 3 | 498 |
| P61020 | RAB5B | 3.5903E−07 | 11 | 2 | 1 | 215 |
| Q8TCB0 | IFI44 | 1.89051E−11 | 9 | 3 | 3 | 444 |
| P20839 | IMPDH1 | 3.79633E−06 | 4 | 2 | 1 | 514 |
| P13010 | XRCC5 | 5.06599E−07 | 11 | 7 | 7 | 732 |
| O15553 | MEFV | 7.96312E−06 | 8 | 4 | 4 | 781 |
| O75934 | BCAS2 | 4.01812E−05 | 24 | 3 | 3 | 225 |
| P20592 | MX2 | 8.82965E−09 | 39 | 23 | 20 | 715 |
| Q9UHI6 | DDX20 | 8.65309E−05 | 4 | 2 | 2 | 824 |
| P05161 | ISG15 | 5.43721E−08 | 37 | 6 | 6 | 165 |
| O14776 | TCERG1 | 0.000167061 | 3 | 3 | 3 | 1098 |
| Q00653 | NFKB2 | 4.3569E−07 | 19 | 11 | 11 | 900 |
| Q16666 | IFI16 | 3.01838E−07 | 27 | 18 | 18 | 785 |
| P09914 | IFIT1 | 4.72041E−07 | 46 | 21 | 21 | 478 |
| O43290 | SART1 | 1.2262E−06 | 47 | 32 | 32 | 800 |
| P52630 | STAT2 | 0.000799226 | 16 | 8 | 1 | 851 |
| P20591 | MX1 | 4.10551E−06 | 39 | 18 | 15 | 662 |
| P04075 | ALDOA | 4.75671E−06 | 68 | 23 | 21 | 364 |
| O95786 | DDX58 | 1.02707E−06 | 9 | 5 | 5 | 925 |
| Q9Y6K5 | OAS3 | 1.61709E−05 | 31 | 29 | 29 | 1087 |
| Q6ZUJ8 | PIK3AP1 | 2.0602E−05 | 7 | 4 | 4 | 805 |
| Q9UII4 | HERC5 | 4.36447E−05 | 12 | 10 | 10 | 1024 |
| P09972 | ALDOC | 3.24465E−05 | 23 | 6 | 4 | 364 |
| P00973 | OAS1 | 3.25013E−05 | 24 | 7 | 7 | 400 |
| Q15646 | OASL | 6.29209E−05 | 41 | 16 | 16 | 514 |
| P29728 | OAS2 | 6.54454E−05 | 38 | 22 | 22 | 719 |
| Q5EBM0 | CMPK2 | 7.62999E−05 | 38 | 10 | 10 | 449 |
| P14598 | NCF1 | 7.74292E−05 | 48 | 14 | 14 | 390 |
| O14933 | UBE2L6 | 4.15319E−05 | 53 | 5 | 5 | 153 |
| Q03169 | TNFAIP2 | 5.10857E−05 | 13 | 5 | 5 | 654 |
| P43490 | NAMPT | 0.000224384 | 27 | 9 | 9 | 491 |
| Q9H3U1-2 | UNC45A | 0.010093224 | 5 | 3 | 3 | 929 |
| Q13459 | MYO9B | 0.008865095 | 2 | 3 | 3 | 2157 |
| P49005 | POLD2 | 0.001568171 | 6 | 2 | 2 | 469 |
| P29508 | SERPINB3 | 0.008370477 | 10 | 4 | 2 | 390 |
| P19474 | TRIM21 | 0.000197083 | 9 | 4 | 4 | 475 |
| Q96GA3 | LTV1 | 0.000423566 | 27 | 12 | 12 | 475 |
| P01889 | HLA-B | 0.000257974 | 20 | 5 | 3 | 362 |
| Q9UGU5 | HMGXB4 | 0.000344271 | 8 | 3 | 3 | 601 |
| Q9UNM6 | PSMD13 | 0.012034188 | 5 | 2 | 2 | 376 |
| P42224 | STAT1 | 0.000826917 | 48 | 35 | 35 | 750 |
| Q96F86 | EDC3 | 0.0183807 | 8 | 2 | 2 | 508 |
| P61158 | ACTR3 | 0.000548364 | 18 | 4 | 4 | 418 |
| P06753-2 | TPM3 | 0.001083333 | 46 | 15 | 8 | 248 |
| Q8IXQ6 | PARP9 | 0.000767085 | 13 | 8 | 8 | 854 |
| O14602 | EIF1AY | 0.008309349 | 34 | 4 | 1 | 144 |
| Q06830 | PRDX1 | 0.001616614 | 76 | 18 | 14 | 199 |
| P23381 | WARS1 | 0.001448813 | 26 | 7 | 7 | 471 |
| Q53G44 | IFI44L | 0.001911125 | 13 | 5 | 5 | 452 |
| P32455 | GBP1 | 0.002863221 | 10 | 4 | 3 | 592 |
| P29692 | EEF1D | 0.002729569 | 33 | 7 | 7 | 281 |
| P18615 | NELFE | 0.006030419 | 16 | 4 | 4 | 380 |
| Q5K651 | SAMD9 | 0.003578333 | 10 | 11 | 11 | 1589 |
| Q8TDB6 | DTX3L | 0.003023628 | 17 | 10 | 10 | 740 |
| P21127-2 | CDK11B | 0.004033685 | 38 | 37 | 4 | 782 |
| P02042 | HBD | 0.005972218 | 18 | 2 | 2 | 147 |
| P28838-2 | LAP3 | 0.004820651 | 51 | 20 | 20 | 488 |
| Q7Z417 | NUFIP2 | 0.004491528 | 3 | 2 | 2 | 695 |
| Q9Y2L1 | DIS3 | 0.003198292 | 4 | 3 | 3 | 958 |
| Q5JTH9 | RRP12 | 0.005569554 | 33 | 33 | 33 | 1297 |
| P32119 | PRDX2 | 0.005593423 | 30 | 8 | 7 | 198 |
| Q8IY81 | FTSJ3 | 0.005673391 | 21 | 14 | 14 | 847 |
| Q9UMW8 | USP18 | 0.005063384 | 15 | 4 | 4 | 372 |
| Q03518 | TAP1 | 0.006299787 | 21 | 11 | 11 | 808 |
| Q9ULV4 | CORO1C | 0.003578922 | 10 | 4 | 4 | 474 |
| Q5TEJ8 | THEMIS2 | 0.006840208 | 22 | 9 | 9 | 643 |
| P19525 | EIF2AK2 | 0.007435898 | 28 | 14 | 14 | 551 |
| P55072 | VCP | 0.008147169 | 33 | 19 | 19 | 806 |
| P40763 | STAT3 | 0.009533038 | 15 | 8 | 8 | 770 |
| Q969Q0 | RPL36AL | 0.009644425 | 45 | 9 | 2 | 106 |
| P19878 | NCF2 | 0.011737652 | 22 | 8 | 8 | 526 |
| Q03519 | TAP2 | 0.010916847 | 16 | 10 | 9 | 686 |
| Q13263 | TRIM28 | 0.008464859 | 6 | 2 | 2 | 835 |
| P53004 | BLVRA | 0.01020831 | 26 | 6 | 6 | 296 |
| Q63HN8 | RNF213 | 0.012908625 | 15 | 60 | 60 | 5207 |
| Q9NUQ6 | SPATS2L | 0.015785522 | 5 | 2 | 2 | 558 |
| O95573 | ACSL3 | 0.01823123 | 18 | 10 | 7 | 720 |
| P52630-4 | STAT2 | 0.014461363 | 16 | 8 | 1 | 847 |
| P23469 | PTPRE | 0.010471086 | 7 | 4 | 4 | 700 |
| P11021 | HSPA5 | 0.01598158 | 42 | 26 | 24 | 654 |
| Q8WWY3 | PRPF31 | 0.010184287 | 6 | 2 | 2 | 499 |
| Q9HB71 | CACYBP | 0.01733789 | 29 | 5 | 5 | 228 |
| P62314 | SNRPD1 | 0.017580861 | 28 | 3 | 3 | 119 |
| P29466 | CASP1 | 0.013091491 | 6 | 3 | 3 | 404 |
| P55265 | ADAR | 0.02000974 | 7 | 7 | 7 | 1226 |
| Q14498-2 | RBM39 | 0.021078052 | 41 | 16 | 15 | 524 |
| P08195 | SLC3A2 | 0.027303718 | 4 | 2 | 2 | 630 |
| Q6UN15 | FIP1L1 | 0.025502679 | 35 | 15 | 15 | 594 |
| P08621 | SNRNP70 | 0.027868718 | 36 | 22 | 22 | 437 |
| Q99459 | CDC5L | 0.028005395 | 44 | 29 | 29 | 802 |
| P40121 | CAPG | 0.032783975 | 7 | 2 | 2 | 348 |
| P12814 | ACTN1 | 0.0325762 | 42 | 31 | 20 | 892 |
| Q68CZ2 | TNS3 | 0.027467904 | 5 | 4 | 4 | 1445 |
| P47756-2 | CAPZB | 0.040814508 | 23 | 5 | 5 | 272 |
| Q8WUM0 | NUP133 | 0.045793019 | 3 | 3 | 3 | 1156 |
| P78344 | EIF4G2 | 0.037068288 | 5 | 4 | 4 | 907 |
| Q66K74 | MAP1S | 0.027302343 | 6 | 4 | 4 | 1059 |
| Q01518 | CAP1 | 0.037763487 | 71 | 37 | 1 | 475 |
| P08574 | CYC1 | 0.041967876 | 4 | 2 | 2 | 325 |
| Q14562 | DHX8 | 0.039470256 | 31 | 28 | 27 | 1220 |
| P09543 | CNP | 0.040997192 | 26 | 8 | 8 | 421 |
| Q9ULA0 | DNPEP | 0.041163727 | 5 | 2 | 2 | 485 |
| O43707 | ACTN4 | 0.045025658 | 35 | 25 | 14 | 911 |
| Q9P2R7 | SUCLA2 | 0.036762317 | 7 | 3 | 3 | 463 |

TABLE 9
IRAK4 proximity analysis

| | | Abundance | | | | |
|---|---|---|---|---|---|---|
| | Gene | Ratio P-Value: | | | # Unique | |
| Accession | Symbol | (DOX)/(NO DOX) | Coverage % | # Peptides | Peptides | # AAs |
| Q13509 | TUBB3 | 1E−17 | 34 | 15 | 1 | 450 |
| P35580 | MYH10 | 1E−17 | 15 | 30 | 7 | 1976 |
| Q58FF8 | HSP90AB2P | 1E−17 | 25 | 10 | 1 | 381 |
| P63000 | RAC1 | 1E−17 | 35 | 8 | 3 | 192 |
| Q9H9B4 | SFXN1 | 1E−17 | 26 | 6 | 5 | 322 |
| Q9UBM7 | DHCR7 | 1E−17 | 4 | 2 | 2 | 475 |
| P29350-2 | PTPN6 | 1E−17 | 58 | 29 | 1 | 556 |
| P84085 | ARF5 | 1E−17 | 22 | 3 | 2 | 180 |
| P62633 | CNBP | 1E−17 | 20 | 3 | 3 | 177 |
| P15311 | EZR | 1E−17 | 14 | 8 | 3 | 586 |
| Q14166 | TTLL12 | 1E−17 | 12 | 5 | 5 | 644 |
| P84095 | RHOG | 1E−17 | 52 | 7 | 6 | 191 |
| P60953 | CDC42 | 1E−17 | 20 | 3 | 2 | 191 |
| P48047 | ATP5PO | 1E−17 | 16 | 3 | 3 | 213 |
| Q13363 | CTBP1 | 1E−17 | 19 | 5 | 5 | 440 |
| Q14004 | CDK13 | 1E−17 | 3 | 3 | 2 | 1512 |
| Q00535 | CDK5 | 1E−17 | 14 | 5 | 4 | 292 |
| Q13630 | GFUS | 1E−17 | 8 | 2 | 2 | 321 |
| P16402 | H1-3 | 1E−17 | 12 | 2 | 2 | 221 |
| P56192 | MARS1 | 1E−17 | 4 | 3 | 3 | 900 |
| Q14847 | LASP1 | 1E−17 | 10 | 2 | 2 | 261 |
| P19338 | NCL | 1E−17 | 5 | 3 | 3 | 710 |
| Q15418 | RPS6KA1 | 1E−17 | 7 | 5 | 5 | 735 |
| P08754 | GNAI3 | 1E−17 | 7 | 3 | 1 | 354 |
| O94973 | AP2A2 | 1E−17 | 13 | 11 | 3 | 939 |
| Q14344 | GNA13 | 1E−17 | 10 | 3 | 2 | 377 |
| P54577 | YARS1 | 1E−17 | 5 | 2 | 2 | 528 |
| Q13057 | COASY | 1E−17 | 9 | 4 | 4 | 564 |
| Q9BXW7 | HDHD5 | 1E−17 | 10 | 3 | 3 | 423 |
| Q9UMR2 | DDX19B | 1E−17 | 4 | 2 | 2 | 479 |
| Q15149 | PLEC | 1E−17 | 3 | 10 | 10 | 4684 |
| P49588 | AARS1 | 1E−17 | 3 | 2 | 2 | 968 |
| Q9Y3A5 | SBDS | 1E−17 | 7 | 2 | 2 | 250 |
| Q92945 | KHSRP | 1E−17 | 3 | 2 | 2 | 711 |
| Q13045 | FLII | 1E−17 | 13 | 13 | 1 | 1269 |
| Q6XQN6 | NAPRT | 1E−17 | 6 | 3 | 3 | 538 |
| P60981 | DSTN | 1E−17 | 15 | 3 | 1 | 165 |
| Q9NVI7 | ATAD3A | 1E−17 | 7 | 4 | 4 | 634 |
| O15260 | SURF4 | 1E−17 | 19 | 4 | 4 | 269 |
| Q9BU23 | LMF2 | 1E−17 | 3 | 2 | 2 | 707 |
| P54819 | AK2 | 1E−17 | 7 | 2 | 2 | 239 |
| P00966 | ASS1 | 1E−17 | 16 | 6 | 6 | 412 |
| O00148 | DDX39A | 1E−17 | 23 | 12 | 1 | 427 |
| Q92608 | DOCK2 | 1E−17 | 13 | 19 | 19 | 1830 |
| P61006 | RAB8A | 1E−17 | 15 | 4 | 1 | 207 |
| P46977 | STT3A | 1E−17 | 6 | 5 | 4 | 705 |
| Q9BW92 | TARS2 | 1E−17 | 5 | 3 | 2 | 718 |
| P45880 | VDAC2 | 1E−17 | 12 | 3 | 3 | 294 |
| P07954 | FH | 1E−17 | 13 | 5 | 5 | 510 |
| P11166 | SLC2A1 | 1E−17 | 6 | 3 | 3 | 492 |
| O00303 | EIF3F | 1E−17 | 18 | 5 | 5 | 357 |
| O43396 | TXNL1 | 1E−17 | 10 | 2 | 2 | 289 |
| O00203 | AP3B1 | 1E−17 | 15 | 15 | 15 | 1094 |
| P52294 | KPNA1 | 1E−17 | 7 | 3 | 2 | 538 |
| O95758-1 | PTBP3 | 1E−17 | 8 | 4 | 1 | 521 |
| P62495 | ETF1 | 1E−17 | 6 | 3 | 3 | 437 |
| O14920 | IKBKB | 1E−17 | 6 | 4 | 4 | 756 |
| Q7Z7A4 | PXK | 1E−17 | 10 | 4 | 4 | 578 |
| Q709C8 | VPS13C | 1E−17 | 1 | 2 | 2 | 3753 |
| Q9BUN8 | DERL1 | 1E−17 | 8 | 2 | 2 | 251 |
| P31689 | DNAJA1 | 1E−17 | 12 | 4 | 4 | 397 |
| Q8N766 | EMC1 | 1E−17 | 3 | 2 | 2 | 993 |
| Q9HCG8 | CWC22 | 1E−17 | 17 | 15 | 15 | 908 |
| O95232 | LUC7L3 | 1E−17 | 29 | 10 | 10 | 432 |
| Q9BQ39 | DDX50 | 1E−17 | 12 | 6 | 5 | 737 |
| Q96AA3 | RFT1 | 1E−17 | 3 | 2 | 2 | 541 |
| Q8IX19 | MCEMP1 | 1E−17 | 11 | 2 | 2 | 187 |
| P55884 | EIF3B | 1E−17 | 3 | 2 | 2 | 814 |
| Q9BR76 | CORO1B | 1E−17 | 6 | 2 | 2 | 489 |
| O95197 | RTN3 | 1E−17 | 3 | 5 | 5 | 1032 |
| Q9NPI1 | BRD7 | 1E−17 | 13 | 4 | 4 | 651 |
| Q9UGP8 | SEC63 | 1E−17 | 14 | 10 | 10 | 760 |
| Q8WVX9 | FAR1 | 1E−17 | 7 | 3 | 3 | 515 |
| A6NDG6 | PGP | 1E−17 | 10 | 3 | 3 | 321 |
| O75683 | SURF6 | 1E−17 | 10 | 3 | 3 | 361 |
| Q9Y6V7 | DDX49 | 1E−17 | 12 | 4 | 4 | 483 |
| Q09161 | NCBP1 | 1E−17 | 9 | 4 | 4 | 790 |
| P13861 | PRKAR2A | 1E−17 | 7 | 2 | 2 | 404 |
| P19784 | CSNK2A2 | 1E−17 | 12 | 4 | 4 | 350 |
| O95218 | ZRANB2 | 1E−17 | 12 | 3 | 3 | 330 |
| O75396 | | 1E−17 | 16 | 3 | 3 | 215 |
| Q02241 | KIF23 | 1E−17 | 3 | 2 | 2 | 960 |
| Q9Y265 | RUVBL1 | 1E−17 | 12 | 4 | 4 | 456 |
| Q8N3C0 | ASCC3 | 1E−17 | 5 | 7 | 7 | 2202 |
| P53992 | SEC24C | 1E−17 | 3 | 2 | 2 | 1094 |
| P85037 | FOXK1 | 1E−17 | 2 | 2 | 2 | 733 |
| O00743 | PPP6C | 1E−17 | 11 | 3 | 3 | 305 |
| Q7L2E3 | DHX30 | 1E−17 | 18 | 16 | 16 | 1194 |
| Q9UMS6 | SYNPO2 | 1E−17 | 2 | 3 | 3 | 1093 |
| Q9BUK6 | MSTO1 | 1E−17 | 4 | 2 | 2 | 570 |
| P08134 | RHOC | 1E−17 | 46 | 6 | 2 | 193 |
| P27361 | MAPK3 | 1E−17 | 6 | 2 | 2 | 379 |
| Q9NW13 | RBM28 | 1E−17 | 5 | 2 | 2 | 759 |
| P22681 | CBL | 1E−17 | 12 | 8 | 8 | 906 |
| Q9UNQ2 | DIMT1 | 1E−17 | 14 | 3 | 3 | 313 |
| Q9NQZ2 | UTP3 | 1E−17 | 23 | 8 | 8 | 479 |
| Q99447 | PCYT2 | 1E−17 | 11 | 3 | 3 | 389 |
| P43243 | MATR3 | 1E−17 | 14 | 7 | 7 | 847 |
| Q13206 | DDX10 | 1E−17 | 7 | 5 | 5 | 875 |
| Q96L92 | SNX27 | 1E−17 | 3 | 2 | 2 | 541 |
| Q13155 | AIMP2 | 1E−17 | 5 | 2 | 2 | 320 |
| O60762 | DPM1 | 1E−17 | 19 | 3 | 3 | 260 |
| P13498 | CYBA | 1E−17 | 13 | 2 | 2 | 195 |
| P62993 | GRB2 | 1E−17 | 17 | 2 | 2 | 217 |
| Q9BXP5 | SRRT | 1E−17 | 2 | 2 | 2 | 876 |
| Q5JVF3 | PCID2 | 1E−17 | 9 | 2 | 2 | 399 |
| Q14980 | NUMA1 | 1E−17 | 3 | 4 | 4 | 2115 |
| P36915 | GNL1 | 1E−17 | 14 | 7 | 7 | 607 |
| Q13464 | ROCK1 | 1E−17 | 3 | 3 | 3 | 1354 |
| P25398 | RPS12 | 1E−17 | 36 | 3 | 3 | 132 |
| Q05519 | SRSF11 | 1E−17 | 15 | 5 | 5 | 484 |
| P08574 | CYC1 | 1E−17 | 4 | 2 | 2 | 325 |
| O60315 | ZEB2 | 1E−17 | 15 | 15 | 15 | 1214 |
| P52907 | CAPZA1 | 1E−17 | 14 | 3 | 3 | 286 |
| Q99439 | CNN2 | 1E−17 | 9 | 2 | 2 | 309 |
| P28340 | POLD1 | 1E−17 | 7 | 7 | 6 | 1107 |
| Q16891 | IMMT | 1E−17 | 3 | 2 | 2 | 758 |
| P62995 | TRA2B | 1E−17 | 27 | 6 | 6 | 288 |
| Q9UJZ1 | STOML2 | 1E−17 | 5 | 2 | 2 | 356 |
| Q8N1F7 | NUP93 | 1E−17 | 5 | 4 | 4 | 819 |
| O14776 | TCERG1 | 1E−17 | 3 | 4 | 4 | 1098 |
| Q6PCB5 | RSBN1L | 1E−17 | 6 | 4 | 3 | 846 |
| Q96JP5 | ZFP91 | 1E−17 | 20 | 10 | 10 | 570 |
| Q5T4S7 | UBR4 | 1E−17 | 1 | 4 | 4 | 5183 |
| O00186 | STXBP3 | 1E−17 | 3 | 2 | 2 | 592 |
| O95347 | SMC2 | 1E−17 | 13 | 12 | 12 | 1197 |
| Q9Y2T2 | AP3M1 | 1E−17 | 15 | 5 | 5 | 418 |
| P62195 | PSMC5 | 1E−17 | 9 | 3 | 2 | 406 |
| Q15382 | RHEB | 1E−17 | 13 | 2 | 2 | 184 |
| O43809 | NUDT21 | 1E−17 | 59 | 14 | 14 | 227 |
| Q9P2R7 | SUCLA2 | 1E−17 | 5 | 3 | 3 | 463 |
| Q6P1X5 | TAF2 | 1E−17 | 10 | 10 | 10 | 1199 |
| P27105 | STOM | 1E−17 | 10 | 2 | 2 | 288 |
| P78316 | NOP14 | 1E−17 | 10 | 7 | 7 | 857 |
| O95239 | KIF4A | 1E−17 | 2 | 3 | 3 | 1232 |
| Q92769 | HDAC2 | 1E−17 | 9 | 3 | 3 | 488 |
| P49593 | PPM1F | 1E−17 | 8 | 2 | 2 | 454 |
| Q9P0U4 | CXXC1 | 1E−17 | 24 | 13 | 13 | 656 |
| P63010 | AP2B1 | 1E−17 | 9 | 6 | 4 | 937 |
| Q14103 | HNRNPD | 1E−17 | 14 | 3 | 3 | 355 |
| Q9NQ55 | PPAN | 1E−17 | 22 | 7 | 7 | 473 |
| Q969V3 | NCLN | 1E−17 | 5 | 3 | 3 | 563 |
| O15269 | SPTLC1 | 1E−17 | 3 | 2 | 2 | 473 |
| O60231 | DHX16 | 1E−17 | 9 | 9 | 8 | 1041 |
| Q15054 | POLD3 | 1E−17 | 13 | 4 | 4 | 466 |
| Q49A26 | GLYR1 | 1E−17 | 9 | 4 | 4 | 553 |
| Q96PU4 | UHRF2 | 1E−17 | 5 | 3 | 2 | 802 |
| Q8TEX9 | IPO4 | 1E−17 | 5 | 3 | 3 | 1081 |
| Q8TBB5 | KLHDC4 | 1E−17 | 10 | 5 | 5 | 520 |
| P52701 | MSH6 | 1E−17 | 4 | 5 | 5 | 1360 |
| Q99549 | MPHOSPH8 | 1E−17 | 8 | 5 | 5 | 860 |
| Q6P2Q9 | PRPF8 | 1E−17 | 13 | 24 | 24 | 2335 |
| O95373 | IPO7 | 1E−17 | 8 | 6 | 6 | 1038 |
| P62140 | PPP1CB | 1E−17 | 18 | 4 | 1 | 327 |
| Q8WXX5 | DNAJC9 | 1E−17 | 23 | 4 | 4 | 260 |
| Q8WXA9-2 | SREK1 | 1E−17 | 15 | 6 | 6 | 624 |
| O95639 | CPSF4 | 1E−17 | 23 | 4 | 4 | 269 |
| Q9UBE0 | SAE1 | 1E−17 | 10 | 2 | 2 | 346 |
| Q9UNX4 | WDR3 | 1E−17 | 12 | 7 | 7 | 943 |
| Q9NQX3 | GPHN | 1E−17 | 3 | 2 | 2 | 736 |
| Q93034 | CUL5 | 1E−17 | 2 | 2 | 2 | 780 |
| Q7L7X3 | TAOK1 | 1E−17 | 18 | 16 | 11 | 1001 |
| Q9NSD9 | FARSB | 1E−17 | 5 | 3 | 3 | 589 |
| P36404 | ARL2 | 1E−17 | 16 | 3 | 3 | 184 |
| Q01433 | AMPD2 | 1E−17 | 3 | 2 | 2 | 879 |
| Q9Y2L1 | DIS3 | 1E−17 | 5 | 4 | 4 | 958 |
| Q9NVP1 | DDX18 | 1E−17 | 19 | 9 | 9 | 670 |
| P0DN79 | CBS | 1E−17 | 16 | 6 | 6 | 551 |
| O75676 | RPS6KA4 | 1E−17 | 2 | 2 | 2 | 772 |
| O43491-4 | EPB41L2 | 1E−17 | 13 | 8 | 7 | 852 |
| Q12872 | SFSWAP | 1E−17 | 10 | 9 | 9 | 951 |
| Q8IYB3 | SRRM1 | 1E−17 | 15 | 12 | 12 | 904 |
| O95352 | ATG7 | 1E−17 | 8 | 5 | 5 | 703 |
| Q92556 | ELMO1 | 1E−17 | 3 | 2 | 1 | 727 |
| P61962 | DCAF7 | 1E−17 | 9 | 2 | 2 | 342 |
| Q9Y383 | LUC7L2 | 1E−17 | 44 | 18 | 12 | 392 |
| O75534 | CSDE1 | 1E−17 | 4 | 3 | 3 | 798 |
| Q13111 | CHAF1A | 1E−17 | 15 | 9 | 9 | 956 |
| Q9BQ70 | TCF25 | 1E−17 | 8 | 3 | 3 | 676 |
| Q8WUA2 | PPIL4 | 1E−17 | 19 | 7 | 7 | 492 |
| Q9UHI6 | DDX20 | 1E−17 | 6 | 3 | 3 | 824 |
| Q9H0C8 | ILKAP | 1E−17 | 15 | 4 | 4 | 392 |
| O75694 | NUP155 | 1E−17 | 4 | 4 | 4 | 1391 |
| Q02880 | TOP2B | 1E−17 | 9 | 12 | 5 | 1626 |
| Q86YP4 | GATAD2A | 1E−17 | 8 | 4 | 3 | 633 |
| O15226 | NKRF | 1E−17 | 3 | 2 | 2 | 690 |
| P26196 | DDX6 | 1E−17 | 13 | 4 | 4 | 483 |
| O00116 | AGPS | 1E−17 | 11 | 5 | 5 | 658 |
| Q96KG9 | SCYL1 | 1E−17 | 3 | 2 | 2 | 808 |
| P11388 | TOP2A | 1E−17 | 19 | 24 | 17 | 1531 |
| Q86VP6 | CAND1 | 1E−17 | 10 | 9 | 9 | 1230 |
| P46926 | GNPDA1 | 1E−17 | 7 | 2 | 2 | 289 |
| Q92620 | DHX38 | 1E−17 | 22 | 22 | 22 | 1227 |
| P27695 | APEX1 | 1E−17 | 28 | 6 | 6 | 318 |
| Q12769 | NUP160 | 1E−17 | 2 | 2 | 2 | 1436 |
| Q92621 | NUP205 | 1E−17 | 5 | 5 | 5 | 2012 |
| P51532 | SMARCA4 | 1E−17 | 20 | 29 | 14 | 1647 |
| Q5T280 | SPOUT1 | 1E−17 | 22 | 6 | 6 | 376 |
| Q4G0F5 | VPS26B | 1E−17 | 7 | 2 | 2 | 336 |
| Q7Z2W4 | ZC3HAV1 | 1E−17 | 21 | 14 | 14 | 902 |
| Q14554 | PDIA5 | 1E−17 | 14 | 6 | 6 | 519 |
| Q9BY44 | EIF2A | 1E−17 | 12 | 6 | 6 | 585 |
| Q1ED39 | KNOP1 | 1E−17 | 5 | 2 | 2 | 458 |
| P35998 | PSMC2 | 1E−17 | 5 | 2 | 2 | 433 |
| P19367 | HK1 | 1E−17 | 3 | 2 | 2 | 917 |
| P00390 | GSR | 1E−17 | 2 | 2 | 2 | 522 |
| Q96EY4 | TMA16 | 1E−17 | 26 | 5 | 5 | 203 |
| Q9BRR9 | ARHGAP9 | 1E−17 | 3 | 2 | 1 | 750 |
| Q8N0V3 | RBFA | 1E−17 | 13 | 2 | 2 | 343 |
| O43684 | BUB3 | 1E−17 | 38 | 11 | 11 | 328 |
| Q96AX1 | VPS33A | 1E−17 | 3 | 2 | 2 | 596 |
| Q96GA3 | LTV1 | 1E−17 | 24 | 10 | 10 | 475 |
| Q9Y230 | RUVBL2 | 1E−17 | 6 | 2 | 2 | 463 |
| Q13131 | PRKAA1 | 1E−17 | 3 | 2 | 2 | 559 |
| Q2M218 | AAK1 | 1E−17 | 8 | 5 | 4 | 961 |
| Q13043 | STK4 | 1E−17 | 5 | 2 | 2 | 487 |
| P26639 | TARS1 | 1E−17 | 7 | 4 | 3 | 723 |
| Q13303 | KCNAB2 | 1E−17 | 8 | 2 | 2 | 367 |
| P28482 | MAPK1 | 1E−17 | 7 | 2 | 2 | 360 |
| Q9H2G2 | SLK | 1E−17 | 7 | 7 | 5 | 1235 |
| Q9HD20 | ATP13A1 | 1E−17 | 2 | 2 | 2 | 1204 |
| P22234 | PAICS | 1E−17 | 14 | 5 | 5 | 425 |
| P40121 | CAPG | 1E−17 | 11 | 3 | 3 | 348 |
| Q5VWQ0 | RSBN1 | 1E−17 | 6 | 5 | 4 | 802 |
| P35236 | PTPN7 | 1E−17 | 15 | 5 | 5 | 360 |
| Q7LDG7 | RASGRP2 | 1E−17 | 4 | 2 | 2 | 609 |
| Q96T37 | RBM15 | 1E−17 | 7 | 5 | 5 | 977 |
| Q9ULA0 | DNPEP | 1E−17 | 5 | 2 | 2 | 485 |
| P56182 | RRP1 | 1E−17 | 25 | 10 | 10 | 461 |
| Q13263 | TRIM28 | 1E−17 | 2 | 2 | 2 | 835 |
| P61160 | ACTR2 | 1E−17 | 5 | 2 | 2 | 394 |
| Q9ULV4 | CORO1C | 1E−17 | 7 | 3 | 3 | 474 |
| Q13310 | PABPC4 | 1E−17 | 6 | 4 | 4 | 644 |
| Q16851 | UGP2 | 1E−17 | 8 | 4 | 4 | 508 |
| Q8WVM8 | SCFD1 | 1E−17 | 9 | 4 | 4 | 642 |
| Q5JTV8 | TOR1AIP1 | 1E−17 | 15 | 7 | 7 | 583 |
| P35232 | PHB1 | 1E−17 | 12 | 3 | 3 | 272 |
| Q96BI1 | SLC22A18 | 1E−17 | 6 | 2 | 2 | 424 |
| P19623 | SRM | 1E−17 | 8 | 2 | 2 | 302 |
| Q9NTI5 | PDS5B | 1E−17 | 15 | 14 | 14 | 1447 |
| P53999 | SUB1 | 1E−17 | 19 | 2 | 2 | 127 |
| Q9H2M9 | RAB3GAP2 | 1E−17 | 2 | 3 | 3 | 1393 |
| Q32MZ4 | LRRFIP1 | 1E−17 | 8 | 6 | 6 | 808 |
| Q12873 | CHD3 | 1E−17 | 9 | 16 | 3 | 2000 |
| P53618 | COPB1 | 1E−17 | 15 | 10 | 10 | 953 |
| Q10567 | | 1E−17 | 6 | 4 | 2 | 949 |
| P13807 | GYS1 | 1E−17 | 5 | 3 | 3 | 737 |
| P09543 | CNP | 1E−17 | 15 | 3 | 3 | 421 |
| Q09666 | AHNAK | 1E−17 | 0 | 2 | 2 | 5890 |
| O14980 | XPO1 | 1E−17 | 3 | 3 | 3 | 1071 |
| P29401 | TKT | 1E−17 | 6 | 3 | 3 | 623 |
| Q15599 | SLC9A3R2 | 1E−17 | 14 | 4 | 4 | 337 |
| Q15910 | EZH2 | 1E−17 | 5 | 4 | 4 | 746 |
| P61009 | SPCS3 | 1E−17 | 11 | 2 | 2 | 180 |
| Q96AG4 | LRRC59 | 1E−17 | 45 | 10 | 10 | 307 |
| Q8WXF1 | PSPC1 | 1E−17 | 8 | 2 | 2 | 523 |
| Q9Y5L0 | TNPO3 | 1E−17 | 8 | 5 | 5 | 923 |
| Q9Y4W6 | AFG3L2 | 1E−17 | 6 | 4 | 4 | 797 |
| P54886 | ALDH18A1 | 1E−17 | 7 | 4 | 4 | 795 |
| Q14289 | PTK2B | 1E−17 | 4 | 3 | 3 | 1009 |
| P32322 | PYCR1 | 1E−17 | 31 | 6 | 5 | 319 |
| Q92878 | RAD50 | 1E−17 | 3 | 3 | 3 | 1312 |
| P51148 | RAB5C | 1E−17 | 28 | 6 | 5 | 216 |
| Q96GM8 | TOE1 | 1E−17 | 12 | 4 | 4 | 510 |
| Q9P210 | CPSF2 | 1E−17 | 14 | 11 | 11 | 782 |
| P50995 | ANXA11 | 1E−17 | 3 | 2 | 2 | 505 |
| A5YKK6 | CNOT1 | 1E−17 | 1 | 2 | 2 | 2376 |
| P0C7P4 | UQCRFS1P1 | 1E−17 | 8 | 2 | 2 | 283 |
| P30041 | PRDX6 | 1E−17 | 40 | 8 | 8 | 224 |
| P53004 | BLVRA | 1E−17 | 21 | 5 | 5 | 296 |
| O95573 | ACSL3 | 1E−17 | 5 | 2 | 1 | 720 |
| O60488 | ACSL4 | 1E−17 | 7 | 4 | 3 | 711 |
| O75530 | EED | 1E−17 | 8 | 4 | 4 | 441 |
| Q9BVJ6 | UTP14A | 1E−17 | 21 | 12 | 12 | 771 |
| P54198 | HIRA | 1E−17 | 2 | 2 | 2 | 1017 |
| Q96P47 | AGAP3 | 1E−17 | 4 | 2 | 2 | 875 |
| Q02447 | SP3 | 1E−17 | 7 | 4 | 4 | 781 |
| Q8TDN6 | BRIX1 | 1E−17 | 16 | 4 | 4 | 353 |
| Q9Y3D8 | AK6 | 1E−17 | 20 | 3 | 3 | 172 |
| P62136 | PPP1CA | 1E−17 | 22 | 5 | 2 | 330 |
| Q15738 | NSDHL | 1E−17 | 6 | 2 | 2 | 373 |
| Q9UBF2 | COPG2 | 0.000125451 | 7 | 5 | 2 | 871 |
| P04075 | ALDOA | 0.0001504 | 78 | 37 | 30 | 364 |
| P47756 | CAPZB | 0.000935619 | 14 | 4 | 4 | 277 |
| Q562R1 | ACTBL2 | 0.006022582 | 28 | 12 | 2 | 376 |
| O95819 | MAP4K4 | 0.002133058 | 13 | 13 | 7 | 1239 |
| P15498 | VAV1 | 0.002357357 | 8 | 7 | 7 | 845 |
| P62191 | PSMC1 | 0.00283967 | 15 | 5 | 4 | 440 |
| Q96GQ7 | DDX27 | 0.009201485 | 22 | 19 | 19 | 796 |
| Q15758 | SLC1A5 | 0.016124136 | 4 | 2 | 2 | 541 |
| P40763 | STAT3 | 0.017905957 | 4 | 2 | 2 | 770 |
| P31939 | ATIC | 0.005211795 | 19 | 8 | 8 | 592 |
| Q13574 | DGKZ | 0.012216345 | 16 | 10 | 10 | 928 |
| Q9UM00 | TMCO1 | 0.007787056 | 15 | 3 | 3 | 239 |
| Q8TCJ2 | STT3B | 0.009837064 | 17 | 14 | 13 | 826 |
| Q9BSC4 | NOL10 | 0.028620099 | 17 | 9 | 9 | 688 |
| Q07955 | SRSF1 | 0.013834443 | 31 | 6 | 6 | 248 |
| Q13148 | TARDBP | 0.021125571 | 8 | 3 | 3 | 414 |
| Q13151 | HNRNPA0 | 0.016882416 | 17 | 3 | 3 | 305 |
| P51531 | SMARCA2 | 0.012775479 | 26 | 33 | 18 | 1590 |
| P22694 | PRKACB | 0.019842981 | 9 | 3 | 3 | 351 |
| Q9HB71 | CACYBP | 0.014701937 | 21 | 5 | 5 | 228 |
| P62891 | RPL39 | 0.015664591 | 24 | 2 | 2 | 51 |
| Q9UG63 | ABCF2 | 0.013275569 | 26 | 16 | 16 | 623 |
| P19447 | ERCC3 | 0.030161367 | 21 | 11 | 11 | 782 |
| Q9H9T3 | ELP3 | 0.049814787 | 12 | 6 | 6 | 547 |
| P18085 | ARF4 | 0.017307436 | 20 | 4 | 3 | 180 |
| Q13557 | CAMK2D | 0.029339722 | 11 | 5 | 4 | 499 |
| Q8NFC6 | BOD1L1 | 0.033747268 | 2 | 5 | 5 | 3051 |
| Q9Y6M1 | IGF2BP2 | 0.024380007 | 17 | 7 | 7 | 599 |
| Q9UKL3 | CASP8AP2 | 0.037438365 | 3 | 5 | 5 | 1982 |
| Q9NX58 | | 0.043779567 | 44 | 20 | 20 | 379 |
| P55160 | NCKAP1L | 0.039190631 | 6 | 5 | 5 | 1127 |
| Q04917 | YWHAH | 0.041368367 | 13 | 4 | 1 | 246 |

TABLE 10
Differential ATAC-seq peaks in MyD88-KO vs WT THP1 cells

| Genes near open peaks | Genes near closed peaks |
|---|---|
| HHAT | FLJ32255 |
| DUSP12 | LY6E |
| EXO1 | ARID1B |
| CUX1 | MIR153-2 |
| CD93 | MIR6071 |
| SAXO1 | EN2 |
| ADAMTS9-AS2 | VAV3 |
| ANGPTL1 | BMP4 |
| TESC | MIR6774 |
| NUDT16P1 | C2orf48 |
| CLNK | IFI27 |
| TESC | LINC00870 |
| BRINP2 | LOC101927817 |
| SEM1 | GLDN |
| PLXNA2 | RFK |
| ANKH | BRDT |
| CCDC60 | |
| MIR3152 | |
| LINC02422 | |
| SLFN12 | |
| IRF6 | |
| ZNF536 | |
| LINC01611 | |
| SLC12A9 | |
| ATP2B4 | |
| MIR3124 | |
| SHCBP1L | |
| HMX3 | |
| PYY2 | |
| SCN7A | |
| MIR548X2 | |
| CNST | |
| PLXNA2 | |
| XXYLT1-AS1 | |
| HPS1 | |
| TEX30 | |
| LOC100131496 | |
| SPAG16 | |
| LINC02270 | |
| TMCO5A | |
| LRRC52 | |
| C11orf87 | |
| ELOVL3 | |
| MAF | |
| LOC100500773 | |
| MIR326 | |
| SOX17 | |
| ST8SIA6 | |
| ZIC4 | |
| TENT5C | |
| ADAMTS9-AS2 | |
| MIR4799 | |
| CLDN10 | |
| GPR148 | |
| SMYD3 | |
| ZNF536 | |
| ZNF568 | |
| DEGS1 | |
| RGS1 | |
| ZP3 | |
| SPOCK1 | |
| ZMAT4 | |
| FMO1 | |
| LINC02055 | |
| GNB4 | |
| UBASH3B | |
| LINC01395 | |
| MIR802 | |
| CD247 | |
| CSMD3 | |
| ADAMTS9 | |
| MICA | |
| LOC285889 | |
| ARL15 | |
| SEC61G | |
| DGKI | |
| LOC101929882 | |
| ZNF471 | |
| OXR1 | |
| FCGR2A | |
| HIF1AN | |
| LINC02032 | |
| LOC101929331 | |
| SEMA5B | |
| MTM1 | |
| GTF3C4 | |
| ZNF599 | |
| GGT1 | |
| MT1G | |
| ASMT | |
| LINC01370 | |
| TAFA1 | |
| SMYD2 | |
| LOC100379224 | |
| DIXDC1 | |
| FOXO6 | |
| THRB | |
| ESYT3 | |
| SLAMF6 | |
| ARX | |
| RGS12 | |
| LINC00971 | |
| LINC01139 | |
| STARD8 | |
| TENT5A | |
| SMYD3 | |
| ADAMTS9-AS1 | |
| KCNJ10 | |
| RBM46 | |
| LOC339685 | |
| MIR3654 | |
| MIR9-3 | |
| TEX30 | |
| MYO16-AS1 | |
| LOC100133091 | |
| LOC101927082 | |
| GNG4 | |
| PROX1-AS1 | |
| ZNF236-DT | |
| CHST15 | |
| EQTN | |
| ANKFN1 | |
| SPOCK1 | |
| FBRSL1 | |
| FAT1 | |
| FAM49A | |
| GPBP1 | |
| MIR548N | |
| LINC01435 | |
| RBM47 | |
| PLXNC1 | |
| RPGRIP1 | |
| ADAMTS9-AS1 | |
| DACH2 | |

TABLE 11
Differential ATAC-seq peaks in IRAK1-KO, IRAK4-KO, and IRAK1/4-dKO vs WT THP 1 cells

| Down genes + Closed peaks | | | Up genes + Open peaks | | |
|---|---|---|---|---|---|
| IRAK1-KO | IRAK4-KO | IRAK1/4-dKO | IRAK1-KO | IRAK4-KO | IRAK1/4-dKO |
| (−MyD88KO) | (−MyD88KO) | (−MyD88KO) | (−MyD88KO) | (−MyD88KO) | (−MyD88KO) |
| vs WT | vs WT | vs WT | vs WT | vs WT | vs WT |
| ANXA4 | AKR1E2 | ADAMTS14 | CMBL | ABCC3 | ABCC3 |
| CA3 | ALG10 | AIFM2 | SLC7A8 | ABHD4 | ADAMTS15 |
| LOC339862 | AMN1 | AKR1E2 | TMEM63C | ABI3 | ALDH1A1 |
| LY9 | ANKRD33B | AMIGO2 | | ADAMTS15 | ALOX5AP |
| PLEKHG5 | ANO6 | ANK3 | | ADRA2B | B3GNT5 |
| RNF165 | APBB1IP | ANKRD33B | | ALDH1A1 | BHLHE22 |
| SLC2A10 | ARID2 | ANO6 | | AOC1 | BMPER |
| UST | ARNTL2 | ARHGAP21 | | ARAP2 | C20orf197 |
| ZFP92 | ARSG | ARNTL2 | | ARHGAP24 | CACFD1 |
| | AVPI1 | ARSG | | ARHGEF3 | CBX3 |
| | BAG3 | BAG3 | | BHLHE22 | CCL3L3 |
| | BEX5 | BEX5 | | C20orf197 | CCT6B |
| | C2CD5 | BRD9 | | C5AR1 | CD14 |
| | C3orf18 | C10orf91 | | CACFD1 | CD55 |
| | CALHM2 | C2CD5 | | CCDC120 | CD82 |
| | CAMK1D | C3orf18 | | CEACAM4 | CDKL5 |
| | CAPRIN2 | CALHM2 | | CEBPE | CDON |
| | CCDC91 | CAMK1D | | CILP2 | CEACAM4 |
| | CCL28 | CAPRIN2 | | CLIP4 | CEBPE |
| | CDC123 | CEP72 | | CORO2A | CILP2 |
| | CES4A | CMAS | | CRLF2 | CKAP4 |
| | CMAS | CNTN1 | | CST7 | CLEC5A |
| | CNTN1 | COL27A1 | | CTSG | CLIP4 |
| | CPNE8 | COL4A5 | | CYP19A1 | CNR1 |
| | CRABP1 | COMTD1 | | CYP1B1 | CPE |
| | CTBP2 | CRABP1 | | CYP27A1 | CRLF2 |
| | CWF19L1 | CTBP2 | | CYP46A1 | CST7 |
| | DACH1 | DDIT4 | | CYP7B1 | CTSG |
| | DAPK2 | DDX11 | | DEFA4 | CYP19A1 |
| | DCLRE1C | DDX11-AS1 | | DMXL2 | CYP1B1 |
| | DDIT4 | DENND5B | | DYSF | CYP27A1 |
| | DDX11 | DNAJB12 | | EML1 | CYP46A1 |
| | DDX11-AS1 | DNM1L | | EPB41L3 | DCAF13 |
| | DENND5B | DNMBP | | EPHB1 | DMXL2 |
| | DNAJC1 | DPY19L2 | | ERG | EPB41L3 |
| | DNAJC21 | DUSP5 | | FBXO15 | EPHA7 |
| | DNM1L | ECHDC3 | | FBXO32 | FAM135B |
| | EGR1 | EIF3A | | FRMD3 | FAM49B |
| | ELK1 | ELK1 | | GP9 | FBXO15 |
| | ETNK1 | FGD4 | | GSG1L | FBXO32 |
| | FAR2 | FGFR3 | | IL12RB2 | FCAR |
| | FGD4 | GOLPH3 | | IL16 | FZD6 |
| | FGFR1OP2 | GPC6 | | KIAA0355 | G2E3 |
| | GLRX3 | GRK5 | | KIT | GADD45A |
| | GPC6 | GTF3C5 | | KLF15 | GP9 |
| | GSTO1 | HMX2 | | KLHL29 | IL16 |
| | HMX2 | HNRNPF | | KRT17 | KIF5C |
| | HSD17B8 | HSD17B8 | | KY | KIT |
| | HTR7 | HSH2D | | LDLRAD3 | KPNA5 |
| | IPO8 | INPP5A | | LINC00452 | LCA5 |
| | ITPR2 | IPO8 | | LINC00565 | LDLRAD3 |
| | JMJD1C | ITPR2 | | LINC00654 | LINC00211 |
| | KCNS3 | JMJD1C | | LOC401463 | LINC00654 |
| | KIF11 | KAZALD1 | | LPO | LOC100287896 |
| | KLHL42 | KCNS3 | | LRRC6 | LOC284240 |
| | KRAS | KLHL42 | | MARCO | LOC401463 |
| | LINC00477 | KRAS | | MFNG | LSP1 |
| | LOC100506688 | LINC00477 | | MGAT4A | LURAP1L |
| | LPCAT1 | LOC100506688 | | MGLL | MARCO |
| | LRMP | LOC648987 | | MITF | MERTK |
| | LZTS2 | LPCAT1 | | MTUS1 | MFNG |
| | MED21 | LRMP | | MX2 | MGAT4A |
| | MPPED2 | MCMDC2 | | MYO7A | MPC1 |
| | MRPS35 | MDGA2 | | NAV1 | MS4A4A |
| | MSX1 | MPPED2 | | NLRC3 | MX2 |
| | NCOA4 | MRPL43 | | NLRP2 | MYO7A |
| | NELL2 | MRPS35 | | NRP2 | MYO7B |
| | NNT | NIM1K | | NT5E | NLRC3 |
| | NNT-AS1 | NRARP | | PAQR5 | NMNAT2 |
| | NOC3L | OGDHL | | PDE9A | NRP2 |
| | OAT | OXCT1 | | PDPN | NT5E |
| | OGDHL | PABPC1L | | PGR | NUAK1 |
| | OXCT1 | PAOX | | PNOC | NUDT16P1 |
| | PABPC1L | PCED1B | | POU4F2 | NWD1 |
| | PAWR | PCED1B-AS1 | | PPP1R13B | PDE4D |
| | PCDHGA12 | PDLIM1 | | PRDM13 | PDE9A |
| | PCED1B | PLEKHA8P1 | | PTCH2 | PDPN |
| | PCED1B-AS1 | PPFIBP1 | | PTK7 | PLCB4 |
| | PDSS1 | PPP2R3A | | RAB27B | POU4F2 |
| | PLEKHA8P1 | PRICKLE1 | | RAB38 | PPP1R14C |
| | PLEKHO1 | PSTPIP2 | | RGS20 | PRDM13 |
| | PPFIBP1 | PWWP2B | | ROS1 | PTCRA |
| | PPP2R2D | RPAP3 | | S100A9 | RAB27B |
| | RASSF8 | SCAF11 | | SASH1 | RASGRF1 |
| | RGS10 | SCD | | SCRN1 | RGCC |
| | RNLS | SEPHS1 | | SH3BGRL2 | RGS20 |
| | RPAP3 | SFMBT2 | | SLAMF9 | RGS5 |
| | SCAF11 | SFXN3 | | SLC11A1 | RHOH |
| | SDHAP3 | SH3PXD2A | | SLC2A10 | ROS1 |
| | SETD6 | SHANK3 | | SLC44A1 | S100A9 |
| | SFMBT2 | SKIDA1 | | SLCO2B1 | SASH1 |
| | SHANK3 | SLC12A7 | | SOWAHC | SENP6 |
| | SLC12A7 | SLC29A3 | | SPATA31C1 | SESN3 |
| | SLC2A13 | SLC38A1 | | SPOCK1 | SH3BGRL2 |
| | SLC35G1 | SMAD1 | | STXBP5-AS1 | SH3TC1 |
| | SLC38A2 | SOX1 | | STXBP6 | SIGLEC7 |
| | SMC3 | SOX5 | | TBL1X | SLC11A1 |
| | SOX1 | STON2 | | TCEA3 | SLC2A10 |
| | SOX5 | TACC2 | | TESC | SLC44A1 |
| | SPEF2 | TAF5 | | TFAP2C | SLC9A7P1 |
| | STK38L | TARS | | TIAM2 | SMYD3 |
| | TARS | TET1 | | TREML2 | SNX10 |
| | TLX1 | TLX1 | | TSGA10 | SOWAHC |
| | TM7SF3 | TM7SF3 | | TSHZ3 | STAT4 |
| | UGT3A2 | TUBGCP2 | | UNC5C | STX11 |
| | UPF2 | UGT3A2 | | WIPF3 | STXBP5 |
| | VENTX | VENTX | | ZBTB18 | STXBP5-AS1 |
| | YARS2 | WDR70 | | ZFHX2 | SUCNR1 |
| | ZFAND4 | YARS2 | | ZNF334 | TBC1D4 |
| | ZNF22 | ZFYVE27 | | ZNF536 | TCTN1 |
| | ZNF320 | ZMIZ1 | | ZSWIM4 | TENM3 |
| | ZNF43 | ZMYND11 | | | TESC |
| | ZNF511 | ZNF43 | | | TLR4 |
| | ZNF572 | ZNF438 | | | TLR5 |
| | ZNF622 | ZNF622 | | | TMEM71 |
| | ZNF99 | ZNF99 | | | TMTC2 |
| | | | | | TREM1 |
| | | | | | TRPM8 |
| | | | | | TSGA10 |
| | | | | | TSHZ3 |
| | | | | | TSPAN18 |
| | | | | | UACA |
| | | | | | UNC5C |
| | | | | | WIPF3 |
| | | | | | ZBTB18 |
| | | | | | ZNF334 |
| | | | | | ZNF37A |
| | | | | | ZNF536 |

TABLE 12
IRAK1/4 signature
Gene name

KRAS
SOX5
HMX2
TACC2
AIFM2
ZNF438
FGD4
GRK5
ANK3
ZNF622
PWWP2B
SFXN3
SLC29A3
COL27A1
ARHGAP21
PDLIM1
CAMK1D
SCAF11
DNAJB12
TUBGCP2
DNMBP
JMJD1C
ELK1
SFMBT2
NRARP
DDIT4
DUSP5
INPP5A
BRD9
ZFYVE27
LRMP
SLC38A1
SLC12A7
ZMIZ1
ANO6
PRICKLE1
CALHM2
LINC00477
NIM1K
COMTD1
ECHDC3
PAOX
LPCAT1
ARSG
PSTPIP2
VENTX
AMIGO2
PCED1B
PCED1B-AS1
BAG3

TABLE 13
CRISPR activation screen in IRAK1/4^{dKO} THP 1 cells

| Gene ID | Score | P value | FDR | Fold change |
|---|---|---|---|---|
| CLEC14A | 1.02E−05 | 3.70E−05 | 0.160891 | 4.7089 |
| B3GNTL1 | 1.28E−05 | 4.53E−05 | 0.160891 | 4.5007 |
| GHDC | 2.13E−05 | 6.95E−05 | 0.160891 | 4.2536 |
| PHGR1 | 1.40E−05 | 4.85E−05 | 0.160891 | 4.0855 |
| PPP1R17 | 6.40E−05 | 0.0002021 | 0.346985 | 3.5241 |
| GNPTAB | 0.0001146 | 0.000352 | 0.385149 | 3.3594 |
| KIFC3 | 0.0001608 | 0.0004904 | 0.385149 | 3.3503 |
| ZNF808 | 0.0003436 | 0.0010277 | 0.412998 | 3.3302 |
| GDF7 | 9.01E−05 | 0.0002718 | 0.36669 | 3.296 |
| FSIP1 | 0.0001999 | 0.0005926 | 0.385149 | 3.2433 |
| MAFF | 0.0002174 | 0.0006513 | 0.385149 | 3.1419 |
| CALCB | 0.0002734 | 0.0008164 | 0.395405 | 3.1385 |
| SPAG5 | 0.0002274 | 0.0006817 | 0.385149 | 3.0663 |
| EQTN | 0.0003259 | 0.0009732 | 0.412998 | 3.0443 |
| TNFAIP3 | 0.000151 | 0.0004642 | 0.385149 | 3.0355 |
| EML1 | 0.0010385 | 0.0029814 | 0.524551 | 3.0353 |
| ZNF729 | 0.0009363 | 0.0026946 | 0.51538 | 3.0345 |
| KDM4C | 0.0007261 | 0.0021028 | 0.51538 | 3.0334 |
| PNMA6A | 2.17E−05 | 7.05E−05 | 0.160891 | 3.0142 |
| DTYMK | 0.0005913 | 0.0017296 | 0.490453 | 3.0106 |
| UNC79 | 0.0009161 | 0.0026343 | 0.51538 | 3.0087 |
| PGAP2 | 0.0001823 | 0.0005501 | 0.385149 | 2.9497 |
| ESYT2 | 0.0007115 | 0.0020562 | 0.51538 | 2.9405 |
| SCARF1 | 0.0015634 | 0.0044397 | 0.539071 | 2.9343 |
| CYTH1 | 0.0007944 | 0.0022962 | 0.51538 | 2.9243 |
| IGF2BP3 | 0.0014081 | 0.0039799 | 0.535455 | 2.9004 |
| RNF212 | 0.0011748 | 0.0033645 | 0.524551 | 2.891 |
| WNT10B | 0.0009179 | 0.002638 | 0.51538 | 2.8872 |
| KIAA0556 | 0.0002547 | 0.0007588 | 0.395405 | 2.8862 |
| THPO | 0.0004955 | 0.0014601 | 0.471807 | 2.8781 |
| SCAMP3 | 0.0004996 | 0.001467 | 0.471807 | 2.8737 |
| GMFB | 0.0030379 | 0.0084781 | 0.6457 | 2.8689 |
| WDR82 | 0.0002949 | 0.0008762 | 0.403646 | 2.8681 |
| ATP5G3 | 0.0023939 | 0.006721 | 0.6126 | 2.8531 |
| RPL24 | 2.23E−05 | 7.16E−05 | 0.160891 | 2.8252 |
| TEX30 | 0.0089099 | 0.023961 | 0.777567 | 2.8217 |
| VPS37B | 0.0004431 | 0.0013181 | 0.467822 | 2.8195 |
| TRIP6 | 0.0003688 | 0.0011021 | 0.425451 | 2.8161 |
| THADA | 0.0007164 | 0.002074 | 0.51538 | 2.8109 |
| LRBA | 0.0018278 | 0.005184 | 0.565988 | 2.8003 |
| MAMLD1 | 0.0009567 | 0.0027491 | 0.51538 | 2.7956 |
| MKNK2 | 0.0021819 | 0.0061407 | 0.606977 | 2.7872 |
| CEP57 | 0.0014605 | 0.0041293 | 0.539071 | 2.7865 |
| BMP1 | 0.0012588 | 0.0035831 | 0.525405 | 2.7834 |
| LOC100130451 | 0.0010327 | 0.0029635 | 0.524551 | 2.756 |
| NCAPG2 | 0.0008961 | 0.0025746 | 0.51538 | 2.7534 |
| ESS2 | 0.0008835 | 0.0025379 | 0.51538 | 2.7509 |
| MBLAC2 | 0.003114 | 0.0086799 | 0.648006 | 2.748 |
| HMX1 | 0.023594 | 0.060282 | 0.867024 | 2.7249 |
| TMC6 | 0.0006202 | 0.001814 | 0.490453 | 2.7239 |
| DVL2 | 0.012503 | 0.033049 | 0.79022 | 2.7167 |
| GIT1 | 0.000841 | 0.0024152 | 0.51538 | 2.7054 |
| GOLGA5 | 0.0003864 | 0.0011488 | 0.425451 | 2.7042 |
| BRAT1 | 0.002107 | 0.0059378 | 0.596561 | 2.6928 |
| FCHO2 | 0.0011318 | 0.0032377 | 0.524551 | 2.6875 |
| ATP6V1G1 | 0.0005884 | 0.0017196 | 0.490453 | 2.6867 |
| ASMTL | 0.000664 | 0.0019361 | 0.500949 | 2.6856 |
| RBM15B | 0.0012296 | 0.0035103 | 0.525405 | 2.6788 |
| PIM3 | 0.0011624 | 0.003331 | 0.524551 | 2.6783 |
| GPATCH2L | 0.0001611 | 0.0004909 | 0.385149 | 2.6614 |
| CNIH4 | 0.0011481 | 0.003287 | 0.524551 | 2.658 |
| SMIM6 | 0.0030709 | 0.0085593 | 0.647923 | 2.6566 |
| NHLRC2 | 0.014125 | 0.026486 | 0.777567 | 2.656 |
| ENPP2 | 0.0047805 | 0.013208 | 0.706967 | 2.6495 |
| REEP1 | 0.0009437 | 0.0027166 | 0.51538 | 2.6456 |
| FRMPD2 | 0.0046784 | 0.012941 | 0.704383 | 2.6451 |
| PTAR1 | 0.0003823 | 0.0011372 | 0.425451 | 2.6406 |
| ESPL1 | 0.0028499 | 0.0079602 | 0.637083 | 2.6404 |
| CENPS-CORT | 0.0032283 | 0.0089745 | 0.649463 | 2.6402 |
| C16orf87 | 0.0015681 | 0.0044502 | 0.539071 | 2.6365 |
| UNKL | 0.0001257 | 0.0003819 | 0.385149 | 2.6349 |
| UBE2E2 | 0.0010857 | 0.0031218 | 0.524551 | 2.6342 |
| PI4KA | 0.033508 | 0.033445 | 0.795614 | 2.6267 |
| ACAD9 | 0.0018873 | 0.0053465 | 0.577058 | 2.625 |
| ATP6V0D1 | 0.0031407 | 0.008758 | 0.649463 | 2.6106 |
| PTPRN2 | 0.0015657 | 0.0044449 | 0.539071 | 2.603 |
| TRAF3IP1 | 0.0010897 | 0.0031308 | 0.524551 | 2.5981 |
| MALSU1 | 0.0008184 | 0.0023597 | 0.51538 | 2.5967 |
| DIAPH2 | 0.0020659 | 0.0058256 | 0.588421 | 2.5848 |
| HIST1H4I | 0.0013434 | 0.0038154 | 0.53381 | 2.5846 |
| DCAF13 | 0.0014217 | 0.0040156 | 0.535455 | 2.5808 |
| WDR73 | 0.0046338 | 0.012824 | 0.700037 | 2.5797 |
| MEIS2 | 0.0001978 | 0.0005895 | 0.385149 | 2.579 |
| PCGF2 | 0.0015515 | 0.0044009 | 0.539071 | 2.5783 |
| ADGRL1 | 2.05E−05 | 6.74E−05 | 0.160891 | 2.5772 |
| DCK | 0.001472 | 0.0041618 | 0.539071 | 2.5726 |
| SRSF7 | 0.0012248 | 0.0034956 | 0.525405 | 2.5706 |
| AP2B1 | 0.0006455 | 0.0018842 | 0.500949 | 2.5697 |
| SULT2B1 | 0.0092908 | 0.02497 | 0.777567 | 2.5621 |
| ABHD5 | 0.0040845 | 0.011281 | 0.673127 | 2.5595 |
| RAMP2 | 0.0001501 | 0.000461 | 0.385149 | 2.5537 |
| C18orf25 | 0.0022449 | 0.0063173 | 0.608785 | 2.5512 |
| TM2D1 | 0.0065734 | 0.017918 | 0.754444 | 2.5478 |
| OR2T2 | 0.03053 | 0.030422 | 0.79022 | 2.5389 |
| RAD23B | 0.0013127 | 0.0037378 | 0.53082 | 2.5356 |
| OR10X1 | 0.0061584 | 0.016843 | 0.745671 | 2.529 |
| LRRC75A | 0.0045573 | 0.012611 | 0.697803 | 2.5243 |
| UBXN7 | 0.0073766 | 0.020021 | 0.77019 | 2.523 |
| PELI3 | 0.0026987 | 0.0075419 | 0.634799 | 2.5188 |
| UTP23 | 0.0089821 | 0.024157 | 0.777567 | 2.5149 |
| POT1 | 0.0020415 | 0.0057669 | 0.586368 | 2.5123 |
| GLB1L2 | 0.0020361 | 0.0057538 | 0.586368 | 2.5104 |
| GTF2H1 | 2.61E−05 | 8.41E−05 | 0.160891 | 2.502 |
| CALD1 | 0.011516 | 0.030604 | 0.79022 | 2.5011 |
| DGAT1 | 0.0037675 | 0.010431 | 0.663716 | 2.4978 |
| MAGEA1 | 0.0034629 | 0.0096004 | 0.654627 | 2.4952 |
| ISLR2 | 0.0040466 | 0.01119 | 0.673127 | 2.4941 |
| ZNF394 | 0.0007507 | 0.002171 | 0.51538 | 2.4938 |
| KAT2A | 0.014909 | 0.039073 | 0.819274 | 2.4887 |
| RNF125 | 0.0010956 | 0.0031465 | 0.524551 | 2.4835 |
| ALG1L2 | 0.0045093 | 0.012469 | 0.697624 | 2.4748 |
| RPUSD4 | 0.0030116 | 0.0084068 | 0.6457 | 2.4736 |
| TAF4B | 0.007637 | 0.020701 | 0.772717 | 2.4703 |
| HSPE1 | 0.0033377 | 0.0092727 | 0.65165 | 2.4695 |
| PRR5L | 0.0026445 | 0.0073956 | 0.632043 | 2.4664 |
| STRN3 | 0.0076628 | 0.020773 | 0.773378 | 2.4654 |
| XRCC5 | 0.0026616 | 0.0074464 | 0.632043 | 2.4652 |
| CORO1C | 0.0020442 | 0.0057743 | 0.586368 | 2.4634 |
| PDZRN3 | 0.0009754 | 0.0027984 | 0.51538 | 2.4633 |
| STK24 | 0.001146 | 0.0032812 | 0.524551 | 2.4613 |
| STX6 | 2.35E−05 | 7.68E−05 | 0.160891 | 2.4572 |
| AKT1 | 0.0034524 | 0.0095721 | 0.654627 | 2.457 |
| SENP7 | 0.0027174 | 0.007588 | 0.634799 | 2.4529 |
| C2orf71 | 0.003484 | 0.0096538 | 0.655905 | 2.452 |
| LTB | 0.0019371 | 0.0054886 | 0.582684 | 2.4517 |
| CCDC130 | 8.91E−05 | 0.0002697 | 0.36669 | 2.4473 |
| RELB | 0.0003222 | 0.0009616 | 0.412998 | 2.4416 |
| ARFGAP1 | 0.0036047 | 0.0099815 | 0.661307 | 2.4381 |
| RABEP1 | 0.001535 | 0.00435 | 0.539071 | 2.4338 |
| TEAD1 | 0.0026216 | 0.0073322 | 0.6295 | 2.4311 |
| C10orf55 | 0.0087882 | 0.02365 | 0.777567 | 2.4307 |
| TIMD4 | 0.0013948 | 0.003949 | 0.535455 | 2.4305 |
| PIKFYVE | 0.0054505 | 0.014991 | 0.739604 | 2.4295 |
| CLDN24 | 0.0086674 | 0.023359 | 0.777567 | 2.4292 |
| KHDC3L | 0.0038594 | 0.010663 | 0.670554 | 2.4264 |
| ANKRD9 | 0.013261 | 0.03494 | 0.807511 | 2.425 |
| GCK | 0.0063548 | 0.017357 | 0.749684 | 2.4237 |
| DDIAS | 0.0032182 | 0.0089493 | 0.649463 | 2.4234 |
| WNT10A | 0.0036694 | 0.01016 | 0.663716 | 2.4222 |
| TNKS2 | 0.0016869 | 0.0047977 | 0.549205 | 2.4203 |
| ZNF792 | 0.0013924 | 0.0039433 | 0.535455 | 2.4202 |
| CES5A | 0.0077767 | 0.021069 | 0.773873 | 2.4149 |
| ETV5 | 0.0023881 | 0.0067094 | 0.6126 | 2.4143 |
| ZNF600 | 0.0033068 | 0.009191 | 0.65165 | 2.4064 |
| EPB41L1 | 0.0072807 | 0.019783 | 0.767286 | 2.406 |
| RAD1 | 0.0059649 | 0.011523 | 0.675896 | 2.4048 |
| FAM118B | 0.015655 | 0.040897 | 0.82361 | 2.404 |
| DSG4 | 0.0093815 | 0.025202 | 0.777567 | 2.4038 |
| CTDSP1 | 0.00866 | 0.023339 | 0.777567 | 2.403 |
| HSDL2 | 0.0027801 | 0.0077636 | 0.634799 | 2.3948 |
| NMNAT2 | 0.021858 | 0.056125 | 0.863212 | 2.3914 |
| GALNT3 | 0.003904 | 0.010791 | 0.670598 | 2.3897 |
| C7orf77 | 0.0062562 | 0.017103 | 0.747765 | 2.3875 |
| FGFR4 | 0.0011327 | 0.0032414 | 0.524551 | 2.3824 |
| MYBL2 | 0.0009556 | 0.0027465 | 0.51538 | 2.3817 |
| EIF1AX | 0.012067 | 0.031978 | 0.79022 | 2.3742 |
| WDR27 | 0.021722 | 0.055805 | 0.861852 | 2.3692 |
| AXDND1 | 0.0041609 | 0.011469 | 0.675896 | 2.3689 |
| RPA1 | 0.0083206 | 0.022482 | 0.777567 | 2.3658 |
| ASIC1 | 0.0034587 | 0.0095878 | 0.654627 | 2.3649 |
| ARHGAP33 | 0.0009777 | 0.0028021 | 0.51538 | 2.3609 |
| OSBPL3 | 0.010952 | 0.029234 | 0.785276 | 2.3607 |
| GLCCI1 | 0.0022354 | 0.0062922 | 0.608785 | 2.3598 |
| MYL5 | 0.0043943 | 0.012128 | 0.690454 | 2.3598 |
| SEMA4D | 0.0005978 | 0.0017474 | 0.490453 | 2.3543 |
| DPYSL2 | 0.0001489 | 0.0004579 | 0.385149 | 2.3542 |
| FBXO34 | 0.0029496 | 0.0082396 | 0.643094 | 2.3506 |
| INTS7 | 0.032591 | 0.032533 | 0.79022 | 2.3497 |
| CDYL | 0.0058966 | 0.016152 | 0.741385 | 2.3473 |
| MORN1 | 0.002777 | 0.0077552 | 0.634799 | 2.3449 |
| DGCR8 | 0.0075293 | 0.020429 | 0.771733 | 2.3437 |
| RASGRP4 | 0.0002003 | 0.0005942 | 0.385149 | 2.3422 |
| NUP210L | 0.0034594 | 0.0095888 | 0.654627 | 2.3353 |
| NPAS3 | 0.0080332 | 0.021746 | 0.777567 | 2.3348 |
| ZKSCAN7 | 0.016277 | 0.042447 | 0.829966 | 2.3327 |
| OR8I2 | 0.01572 | 0.041068 | 0.823933 | 2.3325 |
| LHX1 | 0.0002655 | 0.0007928 | 0.395405 | 2.3278 |
| BRD4 | 0.0056491 | 0.0155 | 0.739604 | 2.3247 |
| GPATCH4 | 0.0009141 | 0.0026301 | 0.51538 | 2.322 |
| NEUROG1 | 0.010714 | 0.028602 | 0.781445 | 2.3204 |
| DSN1 | 0.0060061 | 0.016451 | 0.743368 | 2.3193 |
| ATP5O | 0.009662 | 0.02591 | 0.777567 | 2.3193 |
| TMEM18 | 0.0080929 | 0.021906 | 0.777567 | 2.3173 |
| IQGAP3 | 0.0015886 | 0.0045094 | 0.539071 | 2.3148 |
| PPP3CB | 0.011347 | 0.0302 | 0.79022 | 2.3126 |
| MAGEB1 | 0.0025911 | 0.0072535 | 0.6295 | 2.3058 |
| PFKP | 0.0025728 | 0.0072016 | 0.6295 | 2.3015 |
| PKNOX1 | 0.0071105 | 0.019339 | 0.765749 | 2.3003 |
| NFKBIE | 0.005587 | 0.015334 | 0.739604 | 2.2996 |
| SEC31A | 0.0051229 | 0.014137 | 0.725274 | 2.2975 |
| TXN2 | 0.0005918 | 0.0017322 | 0.490453 | 2.2967 |
| CNNM2 | 0.0016669 | 0.0047416 | 0.546094 | 2.2961 |
| AXIN1 | 0.0001948 | 0.0005842 | 0.385149 | 2.2956 |
| COPB2 | 0.0068053 | 0.018546 | 0.759768 | 2.2905 |
| KIF26B | 0.0063643 | 0.017385 | 0.749684 | 2.2883 |
| TMEM79 | 0.0050042 | 0.013803 | 0.720188 | 2.2862 |
| C14orf80 | 0.0066792 | 0.018195 | 0.755293 | 2.2859 |
| UBE2E1 | 0.0097603 | 0.02617 | 0.777567 | 2.2813 |
| TSPAN15 | 0.013646 | 0.035908 | 0.811054 | 2.281 |
| LPL | 0.0091386 | 0.024571 | 0.777567 | 2.2803 |
| DDX55 | 0.0013433 | 0.0038148 | 0.53381 | 2.2799 |
| LAPTM4B | 0.006596 | 0.017974 | 0.754444 | 2.2755 |
| ZC3H15 | 0.0065447 | 0.01784 | 0.754444 | 2.271 |
| SAAL1 | 0.018572 | 0.048081 | 0.845235 | 2.2674 |
| TSPAN18 | 0.0061863 | 0.016916 | 0.745671 | 2.2673 |
| GINS4 | 0.0063596 | 0.017369 | 0.749684 | 2.2661 |
| ADAMTS18 | 0.019222 | 0.049683 | 0.846385 | 2.266 |
| TEX14 | 0.029509 | 0.074212 | 0.877552 | 2.265 |
| YIPF2 | 0.00577 | 0.01583 | 0.739604 | 2.2617 |
| NFYC | 0.0087332 | 0.023515 | 0.777567 | 2.258 |
| TMEM241 | 0.0080016 | 0.021666 | 0.777567 | 2.2576 |
| ARL15 | 0.00957 | 0.025662 | 0.777567 | 2.2559 |
| MAML2 | 0.0169 | 0.043986 | 0.83573 | 2.2548 |
| FAM153A | 0.016813 | 0.043774 | 0.83573 | 2.2538 |
| OCRL | 0.0003369 | 0.0010083 | 0.412998 | 2.2531 |
| INTS11 | 0.0019465 | 0.0055232 | 0.582803 | 2.2525 |
| TMC5 | 0.0079071 | 0.021421 | 0.776982 | 2.2525 |
| CCDC173 | 0.0084391 | 0.02278 | 0.777567 | 2.2515 |
| ZNF184 | 0.0082617 | 0.02233 | 0.777567 | 2.2511 |
| TVP23B | 0.0002238 | 0.0006712 | 0.385149 | 2.2504 |
| MTBP | 0.0068216 | 0.018585 | 0.759768 | 2.2493 |
| RD3L | 0.021587 | 0.055479 | 0.86087 | 2.249 |
| KREMEN1 | 0.010684 | 0.028525 | 0.781445 | 2.2482 |
| ITPKC | 0.011793 | 0.031289 | 0.79022 | 2.2472 |
| NDST3 | 0.000273 | 0.0008149 | 0.395405 | 2.2446 |
| FGFR1OP | 0.0041761 | 0.01151 | 0.675896 | 2.2446 |
| SNRK | 0.0028308 | 0.0079009 | 0.637083 | 2.2398 |
| SLC3A2 | 0.0040013 | 0.011079 | 0.673127 | 2.2391 |
| ZNF592 | 0.046981 | 0.11346 | 0.928334 | 2.2385 |
| USP17L3 | 0.0015753 | 0.0044737 | 0.539071 | 2.2384 |
| PROK1 | 0.0040836 | 0.011276 | 0.673127 | 2.2371 |
| ENDOG | 0.012776 | 0.033743 | 0.796665 | 2.2364 |
| AADAC | 0.037471 | 0.0923 | 0.900037 | 2.236 |
| GRSF1 | 0.0055826 | 0.01532 | 0.739604 | 2.2348 |
| SLC3A1 | 0.0044496 | 0.012305 | 0.691699 | 2.2342 |
| KIAA2013 | 0.0058512 | 0.016044 | 0.741385 | 2.2324 |
| HORMAD2 | 0.016064 | 0.041914 | 0.826598 | 2.2322 |
| EMC1 | 0.018194 | 0.033719 | 0.796665 | 2.2311 |
| CDC42 | 0.0014762 | 0.0041723 | 0.539071 | 2.2287 |
| HIST1H2BN | 0.0076216 | 0.020666 | 0.772717 | 2.2279 |
| FBXO3 | 0.011604 | 0.030821 | 0.79022 | 2.2213 |
| MTA3 | 0.0040277 | 0.011146 | 0.673127 | 2.2191 |
| CCDC107 | 0.0046257 | 0.0128 | 0.700037 | 2.2184 |
| SYAP1 | 0.0001555 | 0.0004762 | 0.385149 | 2.218 |
| CACFD1 | 0.0011758 | 0.0033672 | 0.524551 | 2.2107 |
| MECP2 | 0.022619 | 0.057931 | 0.865981 | 2.2103 |
| MUT | 0.0037092 | 0.010266 | 0.663716 | 2.21 |
| M6PR | 0.015058 | 0.039441 | 0.819274 | 2.2092 |
| GLTP | 0.0059057 | 0.016172 | 0.741385 | 2.2075 |
| EDIL3 | 0.025174 | 0.064084 | 0.870595 | 2.2053 |
| ABHD13 | 0.0004666 | 0.001393 | 0.469855 | 2.2013 |
| RPS21 | 0.010769 | 0.028754 | 0.781445 | 2.201 |
| SELENOM | 0.018057 | 0.046863 | 0.841908 | 2.199 |
| FXN | 0.031355 | 0.078427 | 0.877706 | 2.1962 |
| ADGRB3 | 0.020372 | 0.0525 | 0.854866 | 2.1948 |
| FBXW2 | 0.010964 | 0.029268 | 0.785276 | 2.1946 |
| ZAR1L | 0.006727 | 0.018314 | 0.756927 | 2.19 |
| CLEC12A | 0.020323 | 0.052371 | 0.854866 | 2.1896 |
| ZNF75D | 0.012357 | 0.032705 | 0.79022 | 2.189 |
| TREML1 | 0.036531 | 0.090172 | 0.893821 | 2.1878 |
| PPP1R8 | 0.0090378 | 0.024308 | 0.777567 | 2.1827 |
| SUPT4H1 | 0.0016395 | 0.0046614 | 0.546094 | 2.1817 |
| STARD3NL | 0.0009809 | 0.0028105 | 0.51538 | 2.1816 |
| VWCE | 0.011415 | 0.030367 | 0.79022 | 2.1802 |
| COL9A3 | 0.011221 | 0.029882 | 0.79022 | 2.1796 |
| AKT2 | 0.0036406 | 0.010082 | 0.663487 | 2.1769 |
| SLC35F5 | 0.0003335 | 0.0009968 | 0.412998 | 2.1749 |
| MAGEA6 | 0.0074986 | 0.02035 | 0.771672 | 2.1714 |
| RASSF3 | 0.000238 | 0.0007106 | 0.385149 | 2.1713 |
| RRAD | 0.013755 | 0.036188 | 0.811054 | 2.169 |
| RILP | 0.016086 | 0.041969 | 0.826598 | 2.1683 |
| SYT2 | 0.0011941 | 0.0034159 | 0.524551 | 2.1669 |
| FKBP3 | 0.0012869 | 0.0036633 | 0.529665 | 2.1669 |
| GBP6 | 0.0091442 | 0.024588 | 0.777567 | 2.1661 |
| CLDN7 | 0.0002394 | 0.0007137 | 0.385149 | 2.166 |
| MLLT10 | 0.012934 | 0.034149 | 0.801261 | 2.1658 |
| ANKRD20A3 | 0.017282 | 0.032091 | 0.79022 | 2.1646 |
| SLC25A12 | 0.0091667 | 0.024647 | 0.777567 | 2.1625 |
| ZNF283 | 0.0068426 | 0.018638 | 0.759768 | 2.1619 |
| SLX4 | 0.027959 | 0.070572 | 0.877552 | 2.1619 |
| TOR3A | 0.017571 | 0.045659 | 0.836807 | 2.1605 |
| LRRC27 | 0.0069719 | 0.018982 | 0.763459 | 2.1596 |
| ZBTB40 | 0.014029 | 0.03688 | 0.812672 | 2.1595 |
| STRADA | 0.0087662 | 0.023589 | 0.777567 | 2.1594 |
| ZNF774 | 0.013998 | 0.036807 | 0.812153 | 2.1578 |
| NDUFA7 | 0.0023676 | 0.0066581 | 0.6126 | 2.1566 |
| DR1 | 0.017679 | 0.045939 | 0.838346 | 2.156 |
| CER1 | 0.01008 | 0.02698 | 0.777567 | 2.1536 |
| CBX3 | 0.0043707 | 0.012064 | 0.690454 | 2.1511 |
| OR5B12 | 0.010751 | 0.028703 | 0.781445 | 2.1508 |
| PYCR2 | 0.010927 | 0.029174 | 0.785276 | 2.1494 |
| HSP90AA1 | 0.0079218 | 0.021465 | 0.776982 | 2.1481 |
| PSMG4 | 0.015771 | 0.04119 | 0.823933 | 2.1465 |
| HCN2 | 0.0099232 | 0.026584 | 0.777567 | 2.1451 |
| IZUMO1 | 0.002485 | 0.0069679 | 0.623739 | 2.1446 |
| HSD17B13 | 0.010087 | 0.026997 | 0.777567 | 2.1432 |
| INF2 | 0.027542 | 0.069601 | 0.877552 | 2.1432 |
| EWSR1 | 0.0027479 | 0.0076792 | 0.634799 | 2.1431 |
| ORM1 | 0.0018665 | 0.0052904 | 0.574286 | 2.1422 |
| WDR63 | 0.000517 | 0.0015152 | 0.474193 | 2.1414 |
| PRPF6 | 0.0003848 | 0.0011456 | 0.425451 | 2.1411 |
| MAVS | 0.0085366 | 0.023026 | 0.777567 | 2.1399 |
| SUV39H2 | 0.0097082 | 0.026027 | 0.777567 | 2.138 |
| MERTK | 0.0057805 | 0.015859 | 0.739604 | 2.1379 |
| CCDC47 | 0.012048 | 0.031927 | 0.79022 | 2.1367 |
| HOGA1 | 0.017281 | 0.044933 | 0.83573 | 2.1365 |
| ZBTB11 | 0.0010812 | 0.0031072 | 0.524551 | 2.1339 |
| FAM102B | 0.010439 | 0.027905 | 0.781445 | 2.1321 |
| SEC11A | 0.0098241 | 0.026332 | 0.777567 | 2.132 |
| GGTLC1 | 0.0066551 | 0.018139 | 0.755293 | 2.1308 |
| C9orf66 | 0.0045975 | 0.012729 | 0.700037 | 2.1307 |
| HTR1B | 0.014043 | 0.036916 | 0.812672 | 2.1269 |
| C11orf98 | 0.0067615 | 0.018418 | 0.757987 | 2.1266 |
| SNPH | 0.0084337 | 0.022764 | 0.777567 | 2.1262 |
| OR8D1 | 0.020843 | 0.053677 | 0.85564 | 2.1256 |
| DKKL1 | 0.0040607 | 0.011223 | 0.673127 | 2.1246 |
| IQCB1 | 0.006713 | 0.018271 | 0.756807 | 2.1221 |
| MFSD6L | 0.0098009 | 0.026283 | 0.777567 | 2.1218 |
| MARF1 | 0.011858 | 0.031458 | 0.79022 | 2.1203 |
| ECH1 | 0.02561 | 0.065108 | 0.872768 | 2.1182 |
| ABLIM1 | 0.016514 | 0.043062 | 0.834217 | 2.1175 |
| SLC35G6 | 0.012074 | 0.031995 | 0.79022 | 2.1168 |
| TMEM99 | 0.012468 | 0.032961 | 0.79022 | 2.1154 |
| SCO2 | 0.0015786 | 0.0044842 | 0.539071 | 2.1153 |
| ATP6V1B1 | 0.022593 | 0.057862 | 0.865981 | 2.1148 |
| CASP8AP2 | 0.016843 | 0.043845 | 0.83573 | 2.1126 |
| SASH3 | 0.0088288 | 0.02375 | 0.777567 | 2.111 |
| RGPD8 | 0.0023303 | 0.0065543 | 0.6126 | 2.1076 |
| ANKRD20A4 | 0.074919 | 0.074926 | 0.877552 | 2.1048 |
| TRNAU1AP | 0.016034 | 0.041835 | 0.826541 | 2.1043 |
| CYB5R1 | 0.02741 | 0.069308 | 0.877552 | 2.1035 |
| VPS39 | 0.015215 | 0.039832 | 0.821334 | 2.1017 |
| ZNF44 | 0.022645 | 0.057989 | 0.865981 | 2.1014 |
| TIMM23 | 0.01498 | 0.039247 | 0.819274 | 2.1011 |
| RSPO2 | 0.016979 | 0.04419 | 0.83573 | 2.1006 |
| SLC28A2 | 0.0088434 | 0.023788 | 0.777567 | 2.0959 |
| GNAO1 | 0.0009654 | 0.0027712 | 0.51538 | 2.0958 |
| SHC4 | 0.0005224 | 0.0015314 | 0.474193 | 2.0955 |
| DDB2 | 0.047249 | 0.11402 | 0.928338 | 2.0949 |
| ALDH3A1 | 0.0063041 | 0.017224 | 0.749589 | 2.0948 |
| TCL1B | 0.028614 | 0.072125 | 0.877552 | 2.0946 |
| FAM178B | 0.0012602 | 0.0035884 | 0.525405 | 2.0935 |
| COQ2 | 0.0042496 | 0.011725 | 0.681447 | 2.0922 |
| TSLP | 0.017633 | 0.045825 | 0.837088 | 2.092 |
| SNTG2 | 0.028898 | 0.072784 | 0.877552 | 2.0919 |
| ME1 | 0.046166 | 0.11167 | 0.926364 | 2.0913 |
| GDF5 | 0.0014001 | 0.0039595 | 0.535455 | 2.0908 |
| UCHL5 | 0.015255 | 0.028528 | 0.781445 | 2.0901 |
| RABGEF1 | 0.0019383 | 0.0054912 | 0.582684 | 2.0897 |
| CHRNG | 0.0062741 | 0.017144 | 0.747845 | 2.0888 |
| MAP3K3 | 0.010384 | 0.02777 | 0.781445 | 2.0885 |
| SLC18A2 | 0.0089189 | 0.023991 | 0.777567 | 2.0879 |
| DDX51 | 0.012086 | 0.032028 | 0.79022 | 2.087 |
| ASPSCR1 | 0.019334 | 0.049952 | 0.846385 | 2.0869 |
| STARD9 | 0.019406 | 0.050114 | 0.846385 | 2.0849 |
| ADAM17 | 0.001084 | 0.0031145 | 0.524551 | 2.0835 |
| CNTN1 | 0.029305 | 0.073737 | 0.877552 | 2.0829 |
| NAB2 | 0.031825 | 0.079491 | 0.879573 | 2.0824 |
| POTED | 0.038803 | 0.07013 | 0.877552 | 2.0816 |
| TAX1BP3 | 0.08669 | 0.086671 | 0.891151 | 2.0815 |
| TINF2 | 0.0055769 | 0.015307 | 0.739604 | 2.0812 |
| INHBB | 0.022559 | 0.057783 | 0.865981 | 2.0811 |
| YBEY | 0.010087 | 0.026997 | 0.777567 | 2.076 |
| SDAD1 | 0.010336 | 0.027632 | 0.781445 | 2.0751 |
| PCSK1N | 0.034924 | 0.086537 | 0.891151 | 2.0747 |
| XRCC2 | 0.013571 | 0.035699 | 0.811054 | 2.0746 |
| SLC48A1 | 0.025455 | 0.064744 | 0.872768 | 2.0744 |
| INTU | 0.0016417 | 0.0046682 | 0.546094 | 2.0724 |
| FN3KRP | 0.01271 | 0.033574 | 0.795725 | 2.0724 |
| OPN3 | 0.0077248 | 0.020941 | 0.773873 | 2.0722 |
| DLK1 | 0.0047355 | 0.013098 | 0.706967 | 2.0718 |
| ARHGAP32 | 0.0077923 | 0.02112 | 0.773873 | 2.0694 |
| TEF | 0.0017607 | 0.0049974 | 0.558212 | 2.0668 |
| ASTE1 | 0.045535 | 0.11029 | 0.926364 | 2.0667 |
| PPIAL4C | 0.005534 | 0.015192 | 0.739604 | 2.0657 |
| FZD5 | 0.017152 | 0.04461 | 0.83573 | 2.0654 |
| IMPA2 | 0.015961 | 0.04167 | 0.825347 | 2.0649 |
| ABCA6 | 0.0078079 | 0.021169 | 0.773873 | 2.0641 |
| TNFAIP8 | 0.009962 | 0.02667 | 0.777567 | 2.0633 |
| CRMP1 | 0.015822 | 0.041317 | 0.824067 | 2.0628 |
| PTS | 0.03168 | 0.079152 | 0.878903 | 2.0623 |
| CNPY2 | 0.02594 | 0.065853 | 0.876559 | 2.0622 |
| TARBP2 | 0.015786 | 0.041224 | 0.823933 | 2.0609 |
| LRP11 | 0.0066199 | 0.018046 | 0.755293 | 2.0599 |
| APOB | 0.010702 | 0.028577 | 0.781445 | 2.0584 |
| RBMXL3 | 0.074449 | 0.17092 | 0.958137 | 2.0581 |
| MOCS1 | 0.03483 | 0.086309 | 0.891151 | 2.0566 |
| ADAMTS10 | 0.035601 | 0.08809 | 0.892638 | 2.0563 |
| ABHD17A | 0.0081338 | 0.022005 | 0.777567 | 2.0516 |
| CROT | 0.010457 | 0.027947 | 0.781445 | 2.0512 |
| ARID3B | 0.0074426 | 0.020196 | 0.771672 | 2.0482 |
| NPAS4 | 0.018817 | 0.048695 | 0.846385 | 2.0474 |
| GINM1 | 0.0012356 | 0.003527 | 0.525405 | 2.0473 |
| ARID5B | 0.013201 | 0.034787 | 0.806214 | 2.0467 |
| DRAXIN | 0.015436 | 0.040365 | 0.82361 | 2.0466 |
| TUBB3 | 0.010348 | 0.027669 | 0.781445 | 2.0451 |
| MS4A6A | 0.011542 | 0.030662 | 0.79022 | 2.0446 |
| SGTA | 0.0074935 | 0.020332 | 0.771672 | 2.0436 |
| CAMKMT | 0.010545 | 0.028165 | 0.781445 | 2.0419 |
| ARMCX5 | 0.010324 | 0.027606 | 0.781445 | 2.0398 |
| ZBP1 | 0.021663 | 0.055664 | 0.861759 | 2.0388 |
| LCE1E | 0.0061677 | 0.01687 | 0.745671 | 2.0382 |
| INAFM2 | 0.0013019 | 0.0037016 | 0.529665 | 2.0379 |
| USP22 | 0.010689 | 0.028542 | 0.781445 | 2.0363 |
| SPDEF | 0.013955 | 0.036708 | 0.812153 | 2.0356 |
| TCF15 | 0.017289 | 0.044954 | 0.83573 | 2.0338 |
| HIST1H3D | 0.088823 | 0.0888 | 0.89281 | 2.0331 |
| TMEM8A | 0.0052574 | 0.014491 | 0.731839 | 2.0324 |
| VDR | 0.012318 | 0.032607 | 0.79022 | 2.032 |
| SPINK1 | 0.032276 | 0.080552 | 0.883201 | 2.0315 |
| RING1 | 0.047065 | 0.11362 | 0.928334 | 2.0314 |
| MFSD8 | 0.0084121 | 0.022717 | 0.777567 | 2.0298 |
| IFNA5 | 0.02043 | 0.052639 | 0.854866 | 2.0283 |
| TM4SF5 | 0.019142 | 0.049489 | 0.846385 | 2.0279 |
| COG5 | 0.023357 | 0.059719 | 0.866014 | 2.0269 |
| ANKRD39 | 0.026708 | 0.067687 | 0.877552 | 2.025 |
| HASPIN | 0.021009 | 0.054084 | 0.856547 | 2.0242 |
| SLC25A33 | 0.01033 | 0.02762 | 0.781445 | 2.0235 |
| TRIM49D2 | 0.031118 | 0.077889 | 0.877552 | 2.0223 |
| TCEAL6 | 0.015873 | 0.041447 | 0.825347 | 2.0214 |
| OSBPL1A | 0.0035631 | 0.0098824 | 0.661307 | 2.0212 |
| ZNF786 | 0.0009176 | 0.0026375 | 0.51538 | 2.0163 |
| CYP11B1 | 0.011179 | 0.029787 | 0.790181 | 2.0147 |
| CLUL1 | 0.000759 | 0.0021982 | 0.51538 | 2.0132 |
| OTUD7B | 0.012151 | 0.032189 | 0.79022 | 2.0123 |
| ADA | 0.0077819 | 0.021087 | 0.773873 | 2.0118 |
| ZBTB16 | 0.024195 | 0.061713 | 0.869622 | 2.0112 |
| GPR149 | 0.0060327 | 0.016515 | 0.743388 | 2.0102 |
| SBSPON | 0.016477 | 0.042975 | 0.833833 | 2.0068 |
| BLOC1S3 | 0.013274 | 0.034975 | 0.807511 | 2.0057 |
| HIST1H2BL | 0.065212 | 0.11429 | 0.928498 | 2.0053 |
| BHMT2 | 0.014987 | 0.039261 | 0.819274 | 2.0025 |
| HMX3 | 0.0026153 | 0.0073164 | 0.6295 | 2.0013 |
| CRTC1 | 0.012093 | 0.032049 | 0.79022 | 2.0013 |
| MUC5AC | 0.014697 | 0.038567 | 0.819274 | 2.001 |
| OPLAH | 0.004112 | 0.011351 | 0.674217 | 2.0006 |
| PIK3R4 | 0.042738 | 0.1041 | 0.915977 | 2.0004 |
| EFCAB6 | 0.02287 | 0.058573 | 0.865981 | 2.0001 |

TABLE 15
AML samples

| | | Age at | |
|---|---|---|---|
| Specimen | Gender | Diagnosis | Karyotype |
| AML-2017- | M | 4 | 46, XY, der(10)?add(10)(p13)ins(10; 11)(p12; q?23q?21), |
| 114 (JM78) | | | ?add(10)(p12), del(11)(q21q23)[14]/47, sl, +8[6] |
| | | | MLL/AF10, PTPN11 |

TABLE 16
Pharmacokinetic analysis of inhibitors

| | Compound 14 | Compound 15 |
|---|---|---|
| Tier 1 Mic t½ (r, min) | >30 | >30 |
| Tier 2 Mic t½ (r/m/h, min) | N.D./25/N.D. | 68/29/102 |
| Plasma Prot Bind (% free - ms, h) | 38.6, 26.5 | 6.85, 2.46 |
| Solubility (mg/mL) | >48 | 31 |
| PAMPA (10⁻⁶ cm/s) | 746 | 495 |

Compound 14 (PO dosing at 30 mg/kg)

| | Mean Total Drug | Mean Free Drug |
|---|---|---|
| Time Point (h) | Concentration (nM) | Concentration (nM) |
| 0.083 | 449 | 173 |
| 0.25 | 869 | 336 |
| 0.5 | 932 | 360 |
| 1 | 801 | 309 |
| 2 | 562 | 217 |
| 4 | 262 | 101 |
| 8 | 78 | 30 |
| 24 | 0 | 0 |

Compound 15 (PO dosing at 100 mg/kg)

| | Mean Total Drug | Mean Free Drug |
|---|---|---|
| Time Point (h) | Concentration (nM) | Concentration (nM) |
| 0.083 | 2672 | 183 |
| 0.25 | 3845 | 263 |
| 0.5 | 3764 | 258 |
| 1 | 3018 | 207 |
| 2 | 2034 | 139 |
| 4 | 1594 | 109 |
| 8 | 726 | 50 |
| 24 | 9 | 1 |

1. A method of treating an inflammatory disease/disorder, acute myeloid leukemia (AML), or myelodysplastic syndrome (MDS) in a subject in need thereof, the method comprising administering to the subject a compound that inhibits IRAK1 and IRAK4.

2. The method of claim 1, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

3. The method of claim 1, wherein the AML is relapsed AML, refractory AML, relapsed/refractory AML, AML with resistance to hypomethylating agents, AML with resistance to venetoclax, AML with resistance to hypomethylating agents and venetoclax, monocytic AML, or monocytic-like AML.

4. The method of claim 1, wherein the MDS is MDS with a splicing factor mutation, MDS with a mutation in isocitrate dehydrogenase 1, or MDS with a mutation in isocitrate dehydrogenase 2.

5. The method of claim 4, wherein the MDS with a splicing factor mutation comprises MDS with a splicing factor mutation in U2AF1, SRSF2, SF3B1, or ZRSR2.

6. The method of any one of claims 1 to 5, wherein the subject in need thereof has elevated IRAK1 expression, IRAK4 expression, FLT3 expression, NF-kB signaling, TLR signaling, IL-1 signaling, or a combination thereof.

7. The method of claim 6, wherein the subject in need thereof has elevated IRAK1 expression, IRAK4 expression, or a combination thereof.

8. The method of claim 6 or 7, wherein the subject in need thereof has elevated FLT3 expression.

9. The method of any one of claims 1 to 8, wherein the compound has an IRAK1 IC₅₀ of less than about 75 nM.

10. The method of any one of claims 1 to 9, wherein the compound has an IRAK4 IC₅₀ of less than about 10 nM.

11. The method of any one of claims 1 to 10, wherein the compound has IRAK4:IRAK1 potency ratio of less than about 40.

12. The method of any one of claims 1 to 11, wherein the subject in need thereof has elevated expression of one or more IRAK1/4-associated genes compared to a healthy control subject and/or decreased expression of one or more IRAK1/4-associated genes compared to a healthy control subject.

13. The method of claim 12, wherein the subject in need thereof has elevated expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

14. The method of claim 12 or 13, wherein the subject in need thereof has decreased expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 compared to the healthy control subject.

15. The method of any one of claims 12 to 14, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

16. The method of claim 15, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38AJ, SLCl2A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

17. The method of claim 15 or 16, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

18. The method of any one of claims 1 to 17, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

19. The method of any one of claims 1 to 18, wherein the compound provides sustained treatment of the inflammatory disease/disorder, AML, or MDS in the subject in need thereof.

20. The method of claim 19, wherein the sustained treatment is greater than the treatment provided from a compound which inhibits IRAK1 without inhibiting IRAK4 or inhibits IRAK4 without inhibiting IRAK1.

21. The method of any one of claims 1 to 20, wherein the compound continues to treat the inflammatory disease/disorder, AML, or MDS in the subject in need thereof after the administration is stopped.

22. The method of claim 21, wherein the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped.

23. The method of any one of claims 1 to 22, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (1-5010), (Ia-5010)-(Id-5010), (II-5010), (IIa-5010), (IIb-5010), (III-5010), (IIIa-5010), (IIIb-5010), or a salt, ester, solvate, optical isomer, geometric isomer, or salt of an isomer of any one thereof.

24. A method of determining effectiveness of a compound in treating an inflammatory disease/disorder, acute myeloid leukemia (AML), or myelodysplastic syndrome (MDS), comprising:
determining the compound's IRAK1 IC₅₀ and IRAK4 IC₅₀;
calculating an IRAK4:IRAK1 potency ratio from the IRAK1 IC₅₀ and the IRAK4 IC₅₀ of the compound;
wherein a potency ratio of less than about 40 is indicative of effectiveness of the compound in treating the inflammatory disease/disorder, AML, or MDS.

25. The method of claim 24, further comprising administering the compound to a subject with an inflammatory disease/disorder, AML, or MDS, treating the inflammatory disease/disorder, AML, or MDS.

26. A method of determining effectiveness of a compound in treating an inflammatory disease/disorder, acute myeloid leukemia (AML), or myelodysplastic syndrome (MDS), comprising:
determining that the compound is an IRAK1/4 inhibitor which modifies the expression of one of more IRAK1/4-associated genes found to be differentially expressed in a subject with an inflammatory disease/disorder, AML, or MDS compared to a healthy control subject.

27. The method of claim 26, further comprising administering the compound to the subject, treating the inflammatory disease/disorder, AIL, or MDS.

28. The method of claim 26 or 27, wherein the compound decreases the expression of one of more IRAK1/4-associated genes found to be elevated in the subject compared to the healthy control subject and/or increases the expression of one of more IRAK1/4-associated genes found to be decreased in the subject compared to the healthy control subject.

29. The method of any one of claims 26 to 28, wherein the compound decreases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

30. The method of any one of claims 26 to 29, wherein the compound increases the expression of one or more IRAK1/4-associated genes selected from: KRAS, SOX5, HMX2, TACC2, AIFM2, ZNF438, FGD4, GRK5, ANK3, ZNF622, PWWP2B, SFXN3, SLC29A3, COL27A1, ARHGAP21, PDLIM1, CAMK1D, SCAF11, DNAJB12, TUBGCP2, DNMBP, JMJD1C, ELK1, SFMBT2, NRARP, DDIT4, DUSP5, INPP5A, BRD9, ZFYVE27, LRMP, SLC38A1, SLC12A7, ZMIZ1, ANO6, PRICKLE1, CALHM2, LINC00477, NIM1K, COMTD, ECHDC3, PAOX, LPCAT1, ARSG, PSTPIP2, VENTX, AMIGO2, PCED1B, PCED1B-AS1, and BAG3 in the subject.

31. The method of any one of claims 24 to 30, wherein the inflammatory disease/disorder is selected from chronic inflammation, sepsis, rheumatoid arthritis, hidradenitis suppurativa, systemic lupus erythematosus, inflammatory bowel disease, multiple sclerosis, psoriasis, Sjögren's syndrome, Ankylosing spondylitis, systemic sclerosis, Type 1 diabetes mellitus, Crohn's disease, and colitis.

32. The method of any one of claims 24 to 30, wherein the AIL is relapsed AML, refractory AML, relapsed/refractory AML, AML with resistance to hypomethylating agents, AML with resistance to venetoclax, AIL with resistance to hypomethylating agents and venetoclax, monocytic AML, or monocytic-like AML.

33. The method of any one of claims 24 to 30, wherein the MDS is MDS with a splicing factor mutation, MDS with a mutation in isocitrate dehydrogenase 1, or MDS with a mutation in isocitrate dehydrogenase 2.

34. The method of claim 33, wherein the MDS with a splicing factor mutation comprises MDS with a splicing factor mutation in U2AF1, SRSF2, SF3B1, or ZRSR2.

35. The method of any one of claims 24 to 34, wherein the compound has an IRAK1 IC₅₀ of less than about 75 nM.

36. The method of any one of claims 24 to 35, wherein the compound has an IRAK4 IC₅₀ of less than about 10 nM.

37. The method of any one of claims 24 to 36, wherein the compound is more effective at inhibiting cancer cell colony formation and/or cancer progenitor cell function when compared to a compound which inhibits IRAK1 without inhibiting IRAK4 or inhibits IRAK4 without inhibiting IRAK1.

38. The method of any one of claims 24 to 37, wherein the compound provides sustained treatment of the inflammatory disease/disorder, AML, or MDS when administered to a subject in need thereof.

39. The method of claim 38, wherein the sustained treatment is greater than the treatment provided from a compound which inhibits IRAK1 without inhibiting IRAK4 or inhibits IRAK4 without inhibiting IRAK1.

40. The method of any one of claims 24 to 39, wherein the compound continues to treat the inflammatory disease/disorder, AML, or MDS in a subject in need thereof after administration of the compound to the subject is stopped.

41. The method of claim 40, wherein the compound continues to treat the inflammatory disease/disorder, AML, or MDS in the subject in need thereof for about 1 day, about 2 days, about 3 days, about 4 days, about 5 days, about 6 days, about one week, about two weeks, about three weeks, or about a month after the administration is stopped.

42. The method of any one of claims 24 to 41, wherein the compound is a compound of any one of Formulas (I-5007), (IIf-5007)-(IIk-5007), (IIm-5007)-(IIt-5007), (IIIq-5007)-(IIIs-5007), (IV-5007), (VI-5003), (VII-5008), (IIf-5008)-(IIk-5008), (IIm-5008), (IIn-5008), (IIp-5008)-(IIu-5008), (IIIq-5008)-(IIIs-5008), (I-5009), (IIf-5009)-(IIj-5009), (1-5010), (Ia-5010)-(Id-5010), (II-5010), (IIa-5010), (IIb-5010), (II-5010), (IIIa-5010), (IIIb-5010), or a salt, ester, solvate, optical isomer, geometric isomer, or salt of an isomer of any one thereof.
